(19) United States
(12) Patent Application Publication
(10) Pub. No.: US 2025/0136607 A1
(43) Pub. Date: May 1, 2025

(54) PAD4 INHIBITORS AND USE THEREOF

(71) Applicant: REGOR PHARMACEUTICALS, INC., Cambridge, MA (US)

(72) Inventors: Lige Li, Shanghai (CN); Baoqi Ren, Shanghai (CN); Wei Wang, Shanghai (CN); Bo Yu, Shanghai (CN); Lei Wu, Shanghai (CN); Wei Guo, Shanghai (CN); Xiaoming Ren, Shanghai (CN); Min Yang, Shanghai (CN); Song Feng, Shanghai (CN); Wenge Zhong, Shanghai (CN); Qingting Meng, Shanghai (CN)

(21) Appl. No.: 18/710,526

(22) PCT Filed: Nov. 15, 2022
(86) PCT No.: PCT/CN2022/131873

§ 371 Date: May 15, 2024

(30) Foreign Application Priority Data
Nov. 15, 2021 (WO) PCT/CN2021/130631

(51) Int. Cl. C07D 487/04; A61K 31/4184; A61K 31/437; A61K 31/4745; A61K 31/4985; A61K 31/5025; A61K 31/538; A61K 31/5415; A61K 31/5517; C07D 471/04; C07D 471/14; C07D 487/14; C07D 498/04; C07D 513/04; C07D 519/00

(52) U.S. Cl. CPC C07D 487/04; A61K 31/4184; A61K 31/437; A61K 31/4745; A61K 31/4985; A61K 31/5025; A61K 31/538; A61K 31/5415; A61K 31/5517; C07D 471/04; C07D 471/14; C07D 487/14; C07D 498/04; C07D 513/04; C07D 519/00

(57) ABSTRACT

The present disclosure provides a compound represented by structural formula (I0) or a pharmaceutically acceptable salt, or a stereoisomer thereof and their use in, e.g. treating a disease or disorder associated with the PAD4 activity. This disclosure also features compositions containing the same as well as methods of using and making the same.

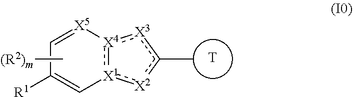

### BACKGROUND

Peptidylarginine deiminases catalyze the posttranslational modification of peptidyl arginine to peptidyl citrulline. There are five known PAD isozymes with 45% to 58% amino acid sequence identity between human isozymes and at least 70% identity across each vertebrate orthologue. PADs have diverse tissue distribution, different putative physiological functions, and reported associations with various disease states. PAD6 is thought to be the only catalytically inactive PAD and is expressed mainly in oocyte, ovary and early embryo; it is proposed to be involved in oocyte cytoskeletal sheet formation and female fertility. PAD1 and PAD3 are expressed in epidermis and hair follicles and are proposed to be involved in cornification of epidermal tissues, hair growth and maintenance of the stratum corneum. PAD2 is expressed more broadly and can be found in multiple tissues and cell types including brain, spinal cord, skeletal muscles, pituitary glands, spleen, neutrophils and macrophages. It is proposed to be involved in plasticity of CNS, transcription regulation, chemokine signaling, and female reproduction.

PAD4 is responsible for the deimination or citrullination of a variety of proteins in vitro and in vivo, with consequences of diverse functional responses in a variety of diseases (Jones J. E. et al, Curr. Opin. Drug Discov. Devel, 12(5), (2009), 616-627). Examples of exemplar diseases include rheumatoid arthritis, diseases with neutrophilic contributions to pathogenesis (for example vasculitis, systemic lupus erythematosus, ulcerative colitis) in addition to oncology indications. PAD4 inhibitors may also have wider applicability as tools and therapeutics for human disease through epigenetic mechanisms.

Inhibitors of PAD4 may have utility against Rheumatoid Arthritis (RA). RA is an autoimmune disease affecting approximately 1% of the population (Wegner N. et al, Immunol. Rev., 233(1) (2010), 34-54). It is characterised by inflammation of articular joints leading to debilitating destruction of bone and cartilage. A weak genetic association between PAD4 polymorphisms and susceptibility to RA has been suggested, albeit inconsistently, in a number of population studies (for example Kochi Y. et al, Ann. Rheum. Dis., 70, (2011), 512-515). PAD4 (along with family member PAD2) has been detected in synovial tissue where it is responsible for the deimination of a variety of joint proteins. This process is presumed to lead to a break of tolerance to, and initiation of immune responses to, citrullinated substrates such as fibrinogen, vimentin and collagen in RA joints. These anti-citrullinated protein antibodies (ACPA) contribute to disease pathogenesis and may also be used as a diagnostic test for RA (e.g. the commercially available CCP2 or cyclic citrullinated protein 2 test). In addition, increased citrullination may also offer additional direct contributions to disease pathogenesis through its ability to affect directly the function of several joint and inflammatory mediators (e.g. fibrinogen, anti-thrombin, multiple chemokines). In a smaller subset of RA patients, anti-PAD4 antibodies can be measured and may correlate with a more erosive form of the disease (Darrah E et al, Sci Transl Med. 2013 May 22; 5(186)).

PAD4 inhibitors may also be useful for the reduction of pathological neutrophil activity in a variety of diseases. Studies suggest that the process of Neutrophil Extracellular Trap (NET) formation, an innate defence mechanism by which neutrophils are able to immobilise and kill pathogens, is associated with histone citrulllination and is deficient in PAD4 knockout mice (Neeli I. et al, J. Immunol, 180, (2008), 1895-1902 and Li P. et al, J. Exp. Med., 207(9), (2010), 1853-1862). PAD4 inhibitors may therefore have applicability for diseases where NET formation in tissues contributes to local injury and disease pathology. Such diseases include, but are not limited to, small vessel vasculitis (Kessenbrock K. et al, Nat. Med, 15(6), (2009), 623-625; Ohlsson S M et al, Clin Exp Immunol. 2014 June; 176(3): 363-72), systemic lupus erythematosus (Hakkim A. et al, Proc. Natl. Acad. Sci. USA, 107(21), (2010), 9813-9818 and Villanueva E. et al, J. Immunol, 187(1), (2011), 538-52), ulcerative colitis (Savchenko A. et al, Pathol. Int., 61(5), (2011), 290-7), cystic fibrosis (Dwyer M et al, J Innate Immun. 2014; 6(6): 765-79), asthma (Dworski R. et al, J. Allergy Clin. Immunol, 127(5), (2011), 1260-6), deep vein thrombosis (Fuchs T. et al, Proc. Natl. Acad. Sci. USA, 107(36), (2010), 15880-5), periodontitis (Vitkov L. et al, Ultrastructural Pathol, 34(1), (2010), 25-30), sepsis (Clark S. R. et al, Nat. Med, 13(4), (2007), 463-9), appendicitis (Brinkmann V. et al, Science, 303, (2004), 1532-5), type 2 diabetes and stroke. In addition, there is evidence that NETs may contribute to pathology in diseases affecting the skin, eg in cutaneous lupus erythematosis (Villanueva E. et al, J. Immunol, 187(1), (2011), 538-52) and psoriasis (Lin A. M. et al, J. Immunol, 187(1), (2011), 490-500), so a PAD4 inhibitor may show benefit to tackle NET skin diseases, when administered by a systemic or cutaneous route. PAD4 inhibitors may affect additional functions within neutrophils and have wider applicability to neutrophilic diseases.

Studies have demonstrated efficacy of tool PAD inhibitors (for example chloro-amidine) in a number of animal models of disease, including collagen-induced arthritis (Willis V. C et al, J. Immunol, 186(7), (2011), 4396-4404), dextran sulfate sodium (DSS)-induced experimental colitis (Chumanevich A. A. et al, Am. J. Physiol. Gastrointest. Liver Physiol, 300(6), (2011), G929-G938), lupus-prone MRL/lpr mice, atherosclerosis and arterial thrombosis (Knight J S et al, Circ Res. 2014 Mar. 14; 114(6):947-56), spinal cord repair (Lange S. et al, Dev. Biol, 355(2), (2011), 205-14), and experimental autoimmune encephalomyelitis (EAE). The DSS colitis report also demonstrates that chloro-amidine drives apoptosis of inflammatory cells both in vitro and in vivo, suggesting that PAD4 inhibitors may be effective more generally in widespread inflammatory diseases.

PAD4 inhibitors may also be useful in the treatment of cancers (Slack. J. L. et al, Cell. Mol. Life Sci., 68(4), (2011), 709-720). Over-expression of PAD4 has been demonstrated in numerous cancers (Chang X. et al, BMC Cancer, 9, (2009), 40). An anti-proliferative role has been suggested for PAD4 inhibitors from the observation that PAD4 citrullinates arginine residues in histones at the promoters of p53-target genes such as p21, which are involved in cell cycle arrest and induction of apoptosis (Li P. et al, Mol. Cell Biol, 28(15), (2008), 4745-4758).

The aforementioned role of PAD4 in deiminating arginine residues in histones may be indicative of a general role for PAD4 in epigenetic regulation of gene expression. PAD4 is the primary PAD family member observed to be resident in the nucleus as well as the cytoplasm. Early evidence that PAD4 may act as a histone demethyliminase as well as a deiminase is inconsistent and unproven. However, it may reduce histone arginine methylation (and hence epigenetic regulation associated with this mark) indirectly via depletion of available arginine residues by conversion to citrulline. PAD4 inhibitors may therefore be useful as epigenetic tools or therapeutics for affecting expression of varied target genes in additional disease settings. PAD4 inhibitors may also be effective in controlling citrullination levels and the switch between pluripotency and differentiation in stem cells (Christophorou M A et al, Nature. 2014 March 6; 507(7490): 104-8) and may therefore therapeutically affect the pluripotency status and differentiation potential of diverse stem cells including, but not limited to, embryonic stem cells, neural stem cells, haematopoietic stem cells and cancer stem cells.

Accordingly, there is a need for inhibitors of PADs that have therapeutic potential in treatment of diseases linked to pathological consequences of citrullination and NETosis including, for example, rheumatoid arthritis, systemic lupus erythematous, antiphospholipid antibody syndrome, small vessels vasculitis, colitis, thrombosis, atherosclerosis, sepsis, diabetes, lung infectious diseases and cancer.

### SUMMARY

Described herein are compounds of formula (I0), pharmaceutically acceptable salts, or stereoisomers thereof:

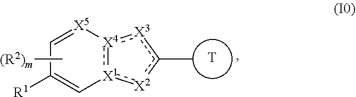

wherein R¹, R², X¹, X², X³, X⁴, X⁵, and ring T are defined herein.

Described herein are compounds of formula (I), pharmaceutically acceptable salts, or stereoisomers thereof:

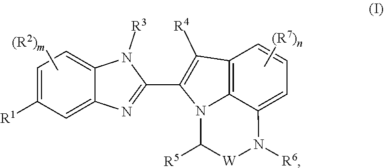

wherein W, R¹, R², R³, R⁴, R⁵, R⁶, R⁷, m, and n are as defined herein.

Also provided are pharmaceutical compositions comprising a compound of formula (I0), (I), (II), (III), (IIIA), (IV), or (V), or a pharmaceutically acceptable salt, or a stereoisomer thereof and a pharmaceutically acceptable carrier or excipient.

The present disclsoure further provides methods of mediating PAD4 in a patient, comprising administering to the patient a compound of formula (I0), (I), (II), (III), (IIIA), (IV), or (V), or a pharmaceutically acceptable salt, or a stereoisomer thereof.

The present disclsoure also provides methods of treating a disease or or condition medidated at least in part by PAD4 in a subject, comprising administering to the subject a therapeutically effective amount of a compound of formula (I0), (I), (II), (III), (IIIA), (IV), or (V), a pharmaceutically acceptable salt, or a stereoisomer thereof.

The present disclosure further provides a method of treating a disease or or condition in a patient in need thereof, comprising administering to the patient an effective amount of (1) a compound of formula (I0), (I), (II), (III), (IIIA), (IV), or (V), a pharmaceutically acceptable salt, or a stereoisomer thereof; or (2) a pharmaceutical composition comprising a compound of formula (I0), (I), (II), (III), (IIIA), (IV), or (V), a pharmaceutically acceptable salt, or a stereoisomer thereof, and a pharmaceutically acceptable carrier; wherein said disease or condition is a bacterial infection, a viral infection, a metabolic disease, an autoimmune disease, an autoinflammatory disease, cancer, or a septic condition.

The present disclosure also provides a use of a compound of formula (I0), (I), (II), (III), (IIIA), (IV), or (V), a pharmaceutically acceptable salt, or a stereoisomer thereof or a pharmaceutical composition comprising the same in any of the methods described herein. In one embodiment, provided is a compound of formula (I0), (I), (II), (III), (IIIA), (IV), or (V), or a pharmaceutically acceptable salt or a stereoisomer thereof or a pharmaceutical composition comprising the same for use in any of the methods described herein. In another embodiment, provided is use of a compound of formula (I0), (I), (II), (III), (IIIA), (IV), or (V), or a pharmaceutically acceptable salt or a stereoisomer thereof or a pharmaceutical composition comprising the same for the manufacture of a medicament for any of the methods described herein.

### DETAILED DESCRIPTION

### 1. Compounds

In a first embodiment, the present disclosure provides a compound of formula (I0):

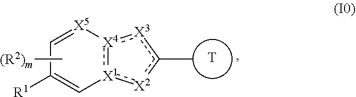

a pharmaceutically acceptable salt, or a stereoisomer thereof, wherein:
[symbol] is a single bond or double bond; provided that

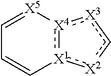

is aromatic;
R¹ is selected from a group consisting of

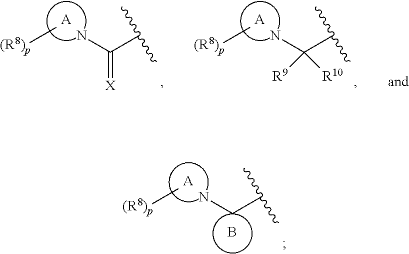

wherein
   X is 0 or S;
   ring A is 4-10 membered heterocyclyl or 5-10 membered heteroaryl;
   ring B is 3-6 membered monocyclic carbocyclyl or 3-6 membered monocyclic heterocyclyl;
R² is deuterium, halogen, CN, C₁₋₆alkyl, C₁₋₆alkoxyl, or —NR^{a}R^{b};
X¹ is N or C;
X² is N;
X³ is —N(R³)— or —C(R³)═;
X⁴ is N or C;
X⁵ is N or CH; wherein
R³ is C₁₋₆alkyl, C₁₋₆alkoxyl, C₂₋₆alkenyl, C₂₋₆alkynyl, —NR^{a}R^{b}, —CH₂-3-8 membered cycloalkyl, —CH₂-3-8 membered heterocyclyl, —CH₂-6-10 membered aryl, or —CH₂-5-10 membered heteroaryl; wherein said C₁₋₆alkyl, C₁₋₆alkoxyl, C₂₋₆alkenyl, C₂₋₆alkynyl, cycloalkyl, heterocyclyl, aryl, or heteroaryl represented by R³ or in the group represented by R³ is optionally substituted with one or more groups selected from halogen, oxo, hydroxyl, C₁₋₆ alkyl, haloC₁₋₆ alkyl, hydoxylC₁₋₆ alkyl, methoxylC₁₋₆ alkyl, C₁₋₆ alkoxyl, haloC₁₋₆ alkoxyl, hydoxylC₁₋₆ alkoxyl, methoxylC₁₋₆ alkoxyl, and —NR^{a}R^{b};
ring T is a tricyclic ring selected from the group consisting of

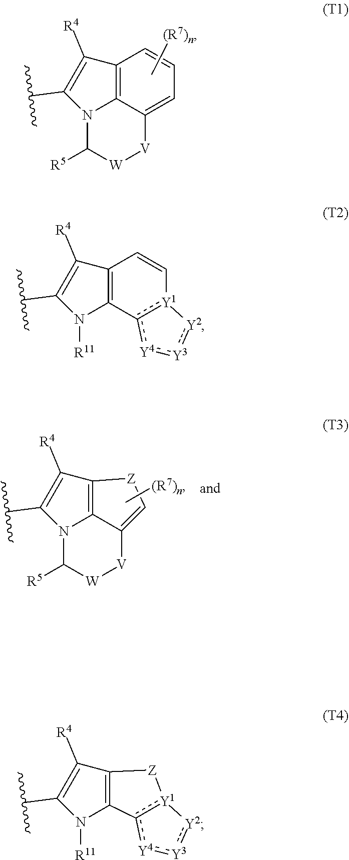

wherein
Z is —O— or —S—;
W is a —(CH₂)ₒ—, —CH(R^{W})—, —C(═O)—, or —CH₂—C(═O)—; wherein o is 1 or 2; R^{W} is C₁₋₆alkyl;
V is —N(R⁶)— or —C(═O)—;
R⁴ is hydrogen, deuterium, halogen, or CN;
R⁵ is hydrogen, C₁₋₆alkyl, haloC₁₋₆alkyl, hydoxylC₁₋₆alkyl, 3-8 membered cycloalkyl, 3-8 membered heterocyclyl, 6-10 membered aryl, or 5-10 membered heteroaryl; wherein said 3-8 membered cycloalkyl, 3-8 membered heterocyclyl, 6-10 membered aryl, or 5-10 membered heteroaryl represented by R⁵ is optionally substituted with one or more groups selected from halogen, oxo, hydroxyl, C₁₋₆ alkyl, haloC₁₋₆ alkyl, hydoxylC₁₋₆ alkyl, methoxylC₁₋₆ alkyl, C₁₋₆ alkoxyl, haloC₁₋₆ alkoxyl, hydoxylC₁₋₆ alkoxyl, methoxylC₁₋₆ alkoxyl, and —NR^{a}R^{b};
R⁶ is hydrogen, C₁₋₆alkyl, C₁₋₆alkylenehydroxyl, C₁₋₆alkyleneamine, benzoyl, carbonylC₁₋₆alkyl, carbonylC₁₋₆alkylenehydroxyl, C₁₋₆alkyleneamide, C₁₋₆alkylenecarbamate, C₁₋₆alkyleneurea, 3-8 membered cycloalkyl, —CH₂-6-10 membered aryl, or —CH₂-5-10 membered heteroaryl; wherein said C₁₋₆alkyl, C₁₋₆alkylenehydroxyl, C₁₋₆alkyleneamine, benzoyl, carbonylC₁₋₆alkyl, carbonylC₁₋₆alkylenehydroxyl, C₁₋₆alkyleneamide, C₁₋₆alkylenecarbamate, C₁₋₆alkyleneurea, 3-8 membered cycloalkyl, —CH₂-6-10 membered aryl, or —CH₂-5-10 membered heteroaryl represented by R⁶ is optionally substituted with one or more groups selected from halogen, hydroxyl, amino, CN, C₁₋₆alkyl, C₁₋₆alkylcarbonyl, C₁₋₆alkylenehydroxyl, C₁₋₆alkylcarbonylamino, and 3-8 membered cycloalkyl;
R⁷ is deuterium, halogen, cyano, C₁₋₆alkyl, C₁₋₆alkoxy, C₂₋₆alkenyl, C₂₋₆ alkynyl, —NR^{a}R^{b}, —S(═O)₂C₁₋₆alkyl, 3-8 membered cycloalkyl, 3-8 membered heterocyclyl, 6-10 membered aryl, or 5-10 membered heteroaryl; wherein said C₁₋₆alkyl, C₁₋₆alkoxy, C₁₋₆alkenyl, C₁₋₆ alkynyl, 3-8 membered cycloalkyl, 3-8 membered heterocyclyl, 6-10 membered aryl, or 5-10 membered heteroaryl represented by R⁷ is optionally substituted with one or more groups selected from halogen and hydroxyl;
Y¹ is C or N; when Y¹ is C, [symbol] is a double bond; and when Y¹ is N, [symbol] is a single bond;
Y² is —O—, —S—, —S(═O)—, —N(R^{d})—, —C(═O)—, —C(R^{d})₂—, or —C(R^{e})═;
Y³ is —CH₂—, —CH₂—CH₂—, —HC═, —NH—, —N═, —C(═O)—, or —N(R^{f})—CH₂—;
Y⁴ is —NH—, —CH₂—, or —N═; wherein
   R^{d} is hydrogen or C₁₋₆alkyl;
   R^{e} is hydrogen, halogen, or C₁₋₆alkyl;
   R^{f} is hydrogen, C₁₋₆alkyl, —C(═O)C₁₋₆alkyl, or 3-6 membered cycloalkyl;
R¹¹ is —CH₂-3-8 membered cycloalkyl;
R⁸ is halogen, CN, C₁₋₆alkyl, haloC₁₋₆alkyl, C₁₋₆alkoxy, —NR^{a}R^{b}, —NR^{a}C(═O)R, —NR^{a}C(═O)OR^{b}, —NR^{a}C(═O)NR^{b}, —NR^{a}SO₂R^{b}, —NR^{a}S(═O)(═NR^{b})R^{c}, 3-8 membered carbocyclyl, or 3-8 membered heterocyclyl; or two R₈ groups together with the atoms they attached form 3-8 membered carbocyclyl or 3-8 membered heterocyclyl;
R⁹ and R¹⁰ are independently hydrogen, deuterium, halogen, C₁₋₆alkyl; wherein said C₁₋₆alkyl is optionally substituted with one or more groups selected from halogen, hydroxyl, and methoxyl;
R^{a}, R^{b}, and R^{c} are each independently selected from the group consisting of hydrogen, deuterium, C₁₋₆alkyl, 3-12 membered carbocyclyl, 3-12 membered heterocyclyl, 6-10 membered aryl, and 5-10 membered heteroaryl;
m and n are independently 0, 1, 2, or 3;
p is 0, 1, 2, 3, 4, 5, or 6; and
wherein said heterocyclyl comprises 1-3 heteroatoms selected from oxygen, nitrogen, and sulfur; and said heteroaryl comprises 1-4 heteroatoms selected from oxygen, nitrogen, and sulfur.

In a second embodiment, the present disclosure provides a compound according to the first embodiment, a pharmaceutically acceptable salt, or a stereoisomer thereof, wherein the compound is represented by formula (I):

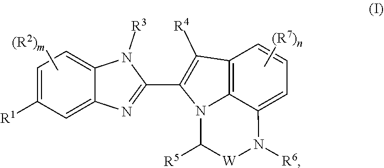

wherein:
W is a —(CH₂)ₒ—, —C(═O)—, or —CH₂—C(═O)—; wherein o is 1, or 2;
R⁷ is deuterium, halogen, cyano, C₁₋₆alkyl, C₁₋₆alkoxy, C₂₋₆alkenyl, C₂₋₆ alkynyl, —NR^{a}R^{b}, 3-8 membered cycloalkyl, 3-8 membered heterocyclyl, 6-10 membered aryl, or 5-10 membered heteroaryl; wherein said C₁₋₆alkyl, C₁₋₆alkoxy, C₁₋₆alkenyl, C₁₋₆ alkynyl, 3-8 membered cycloalkyl, 3-8 membered heterocyclyl, 6-10 membered aryl, or 5-10 membered heteroaryl represented by R⁷ is optionally substituted with one or more groups selected from halogen and hydroxyl. The definitions of the other variables are provided in the first embodiment.

In a third embodiment, the present disclosure provides a compound according to the second embodiment, a pharmaceutically acceptable salt, or a stereoisomer thereof, wherein R¹ is

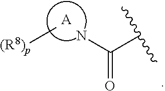

The definitions of the other variables are provided in the second embodiment or formula (I0).

In a fourth embodiment, the present disclosure provides a compound according to the second embodiment, a pharmaceutically acceptable salt, or a stereoisomer thereof, wherein R¹ is

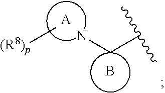

and ring B is 3-4 membered monocyclic heterocyclyl, preferably ring B is oxetanyl. The definitions of the other variables are provided in the second embodiment or formula (I0).

In a fifth embodiment, the present disclosure provides a compound according to the second embodiment, a pharmaceutically acceptable salt, or a stereoisomer thereof, wherein R¹ is

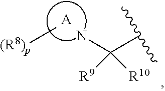

R⁹ and R¹⁰ are independently hydrogen, halo, or haloC₁₋₆alkyl. The definitions of the other variables are provided in the second embodiment or formula (I0).

In a sixth embodiment, the present disclosure provides a compound according to any one of the second through fifth embodiments, a pharmaceutically acceptable salt, or a stereoisomer thereof, wherein W is —CH₂—. The definitions of the other variables are provided in the second through fifth embodiments or formula (I0).

In a seventh embodiment, the present disclosure provides a compound according to any one of the second through sixth embodiments, a pharmaceutically acceptable salt, or a stereoisomer thereof, wherein ring A is 4-6 membered monocyclic heterocyclyl, 6-9 membered fused heterocyclyl, 6-9 membered bridged heterocyclyl, or 6-9 membered spiro heterocyclyl. The definitions of the other variables are provided in the second through sixth embodiments or formula (I0).

In an eighth embodiment, the present disclosure provides a compound according to any one of the second through seventh embodiments, a pharmaceutically acceptable salt, or a stereoisomer thereof, wherein ring A is selected from a group consisting of

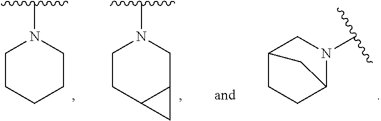 [SMILES: *N1CCCCC1 ; *N1CCC2CC2C1 ; *N1CC2CCC1C2]

The definitions of the other variables are provided in the second through seventh embodiments or formula (I0).

In a ninth embodiment, the present disclosure provides a compound according to any one of the second through eighth embodiments, a pharmaceutically acceptable salt, or a stereoisomer thereof, wherein R⁸ is halogen, C₁₋₆alkyl, haloC₁₋₆alkyl, —NR^{a}R^{b}, —NR^{a}(C═O)R^{b}, or —NR^{a}C(═O)OR^{b}; and p is 0, 1, 2, or 3. The definitions of the other variables are provided in the second through eighth embodiments or formula (I0).

In a tenth embodiment, the present disclosure provides a compound according to any one of the second through ninth embodiments, a pharmaceutically acceptable salt, or a stereoisomer thereof, wherein R⁸ is halogen, NH₂, or C₁₋₃alkyl; and p is 0, 1, or 2. The definitions of the other variables are provided in the second through ninth embodiments or formula (I0).

In an eleventh embodiment, the present disclosure provides a compound according to any one of the second through tenth embodiments, a pharmaceutically acceptable salt, or a stereoisomer thereof, wherein R² is halogen, CN, C₁₋₆alkyl, or C₁₋₆alkoxyl; and m is 0, 1, or 2. The definitions of the other variables are provided in the second through tenth embodiments or formula (I0). In one embodiment, m is 1 and R² is at the meta position to R¹.

In a twelfth embodiment, the present disclosure provides a compound according to any one of the second through eleventh embodiments, a pharmaceutically acceptable salt, or a stereoisomer thereof, wherein R² is —F or —OCH₃; and m is 1. The definitions of the other variables are provided in the second through eleventh embodiments or formula (I0). In one embodiment, m is 1 and R² is at the meta position to R¹; and R² is F.

In a thirteenth embodiment, the present disclosure provides a compound according to the second through twelfth embodiments, a pharmaceutically acceptable salt, or a stereoisomer thereof, wherein R³ is C₁₋₄alkyl, C₁₋₄alkoxyl, C₂₋₄alkynyl, —CH₂-3-5 membered cycloalkyl, —CH₂-3-5 membered heterocyclyl, —CH₂-phenyl, or —CH₂-5-6 membered heteroaryl; wherein said C₁₋₄alkyl, C₁₋₄alkoxyl, C₁₋₄alkynyl, cycloalkyl, heterocyclyl, phenyl, or heteroaryl represented by R³ or in the group represented by R³ is optionally substituted with one to three groups selected from halogen, C₁₋₄ alkyl, hydroxyl, and C₁₋₄alkoxyl. The definitions of the other variables are provided in the second through twelfth embodiments or formula (I0). In one embodiment, R³ is C₁₋₄alkyl.

In a fourteenth embodiment, the present disclosure provides a compound according to the any one of the second through thirteenth embodiments, a pharmaceutically acceptable salt, or a stereoisomer thereof, wherein R³ is C₁₋₂alkyl, C₂₋₃alkynyl, —CH₂-3-4 membered cycloalkyl, —CH₂-3-4 membered heterocyclyl, —CH₂-phenyl, or —CH₂-5 membered heteroaryl; wherein said C₁₋₂alkyl, C₁₋₂alkoxyl, C₂₋₃alkynyl, cycloalkyl, heterocyclyl, phenyl, or heteroaryl represented by R³ or in the group represented by R³ is optionally substituted with one to three groups selected from halogen, C₁₋₂ alkyl, and C₁₋₂alkoxyl. The definitions of the other variables are provided in the second through thirteenth embodiments or formula (I0).

In a fifteenth embodiment, the present disclosure provides a compound according to any one of the second through fourteenth embodiments, a pharmaceutically acceptable salt, or a stereoisomer thereof, wherein R³ is selected from a group consisting of

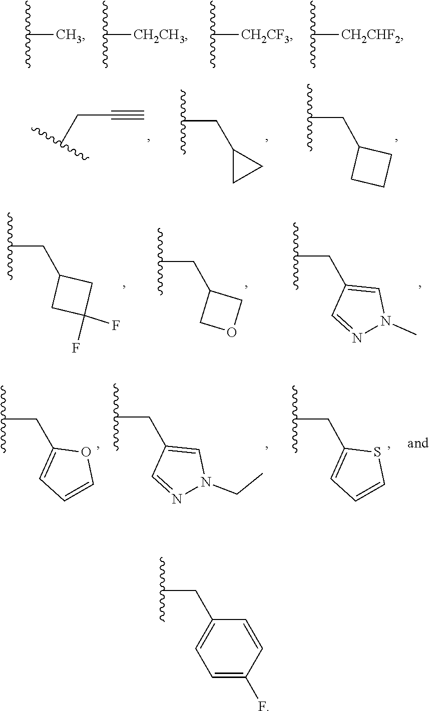

The definitions of the other variables are provided in the second through fourteenth embodiments or formula (I0).

In a sixteenth embodiment, the present disclosure provides a compound according to any one of the second through fifteenth embodiments, a pharmaceutically acceptable salt, or a stereoisomer thereof, wherein R⁴ is hydrogen. The definitions of the other variables are provided in the second through fifteenth embodiments or formula (I0).

In a seventeenth embodiment, the present disclosure provides a compound according to any one of the second through sixteenth embodiments, a pharmaceutically acceptable salt, or a stereoisomer thereof, wherein R⁵ is hydrogen, C₁₋₄alkyl, 3-6 membered cycloalkyl, 3-6 membered heterocyclyl, phenyl, or 5-6 membered heteroaryl, wherein said 3-6 membered cycloalkyl, 3-6 membered heterocyclyl, phenyl, or 5-6 membered heteroaryl represented by R⁵ is optionally substituted with one to three groups selected from halogen, hydroxyl, C₁₋₄ alkyl, haloC₁₋₄ alkyl, hydoxylC₁₋₄ alkyl, methoxylC₁₋₆ alkyl, C₁₋₆ alkoxyl, haloC₁₋₆ alkoxyl, hydoxylC₁₋₆ alkoxyl, methoxylC₁₋₆ alkoxyl, and —NR^{a}R^{b}. The definitions of the other variables are provided in the second through sixteenth embodiments or formula (I0).

In an eighteenth embodiment, the present disclosure provides a compound according to the any one of the second through seventeenth embodiments, a pharmaceutically acceptable salt, or a stereoisomer thereof, wherein R⁵ is hydrogen, C₁₋₃alkyl, or 3-4 membered cycloalkyl. The definitions of the other variables are provided in the second through seventeenth embodiments or formula (I0).

In a ninteenth embodiment, the present disclosure provides a compound according to the any one of the second through eighteenth embodiments, a pharmaceutically acceptable salt, or a stereoisomer thereof, wherein R⁶ is hydrogen, C₁₋₄alkyl, C₁₋₄alkylenehydroxyl, C₁₋₄alkyleneamine, benzoyl, carbonylC₁₋₄alkyl, carbonylC₁₋₄alkylenehydroxyl, C₁₋₄alkyleneamide, C₁₋₄alkylenecarbamate, C₁₋₄alkyleneurea, 3-6 membered cycloalkyl, —CH₂-6 membered aryl, or —CH₂-5-8 membered heteroaryl; wherein said C₁₋₄alkyl, C₁₋₄alkylenehydroxyl, C₁₋₄alkyleneamine, benzoyl, carbonylC₁₋₄alkyl, carbonylC₁₋₄alkylenehydroxyl, C₁₋₄alkyleneamide, C₁₋₄alkylenecarbamate, C₁₋₄alkyleneurea, 3-6 membered cycloalkyl, —CH₂-6 membered aryl, or —CH₂-5-8 membered heteroaryl represented by R⁶ is optionally substituted with one or more groups selected from halogen, hydroxyl, amino, CN, C₁₋₄alkyl, C₁₋₅alkylcarbonyl, C₁₋₄alkylenehydroxyl, C₁₋₄alkylcarbonylamino, and 3-6 membered cycloalkyl. The definitions of the other variables are provided in the second through eighteenth embodiments or formula (I0).

In a twentieth embodiment, the present disclosure provides a compound according to the any one of the second through ninteenth embodiments, a pharmaceutically acceptable salt, or a stereoisomer thereof, wherein R⁶ is hydrogen, C₁₋₃alkyl, C₁₋₃alkylenehydroxyl, C₁₋₃alkyleneamine, benzoyl, carbonylC₁₋₃alkyl, carbonylC₁₋₃alkylenehydroxyl, C₁₋₃alkyleneamide, C₁₋₃alkylenecarbamate, C₁₋₃alkyleneurea, 3-5 membered cycloalkyl, —CH₂-6 membered aryl, or —CH₂-5 membered heteroaryl; wherein said hydrogen, C₁₋₃alkyl, C₁₋₃alkylenehydroxyl, C₁₋₃alkyleneamine, benzoyl, carbonylC₁₋₃alkyl, carbonylC₁₋₃alkylenehydroxyl, C₁₋₃alkyleneamide, C₁₋₃alkylenecarbamate, C₁₋₃alkyleneurea, 3-5 membered cycloalkyl, —CH₂-6 membered aryl, or —CH₂-5 membered heteroaryl represented by R⁶ is optionally substituted with one to three groups selected from fluoro, hydroxyl, amino, CN, C₁₋₃alkyl, C₁₋₅alkylcarbonyl, C₁₋₃alkylenehydroxyl, C₁₋₃alkylcarbonylamino, and 3-4 membered cycloalkyl. The definitions of the other variables are provided in the second through ninteenth embodiments or formula (I0). In one embodiment, R⁶ is hydrogen or C₁₋₃alkylenehydroxyl.

In a twenty-first embodiment, the present disclosure provides a compound according to the any one of the second through twentieth embodiments, a pharmaceutically acceptable salt, or a stereoisomer thereof, wherein R⁶ is selected from a group consisting of

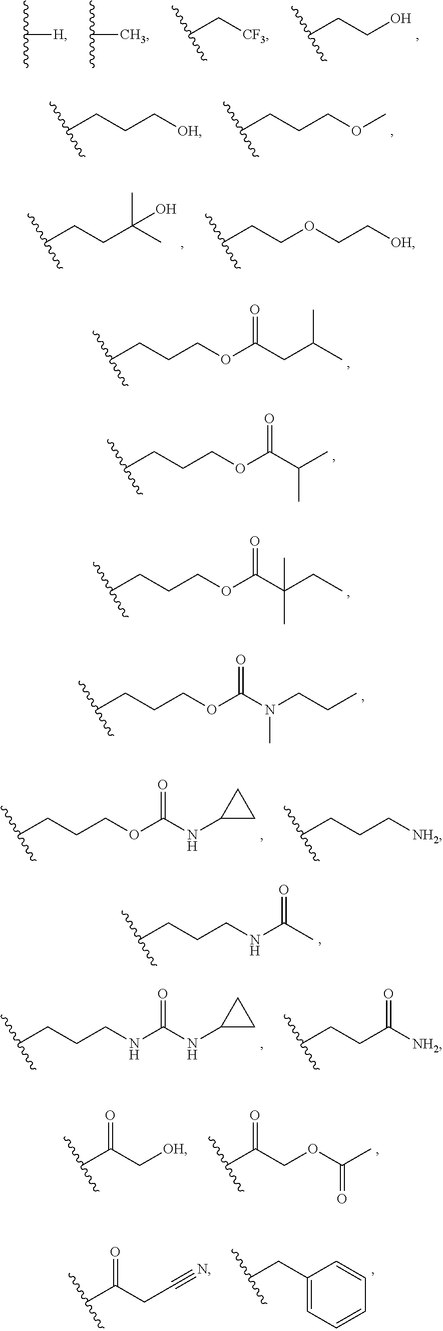

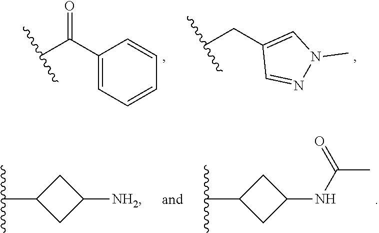

The definitions of the other variables are provided in the second through twentieth embodiments or formula (I0).

In a twenty-second embodiment, the present disclosure provides a compound according to the any one of the second through twenty-first embodiments, a pharmaceutically acceptable salt, or a stereoisomer thereof, wherein R⁶ is selected from a group consisting of

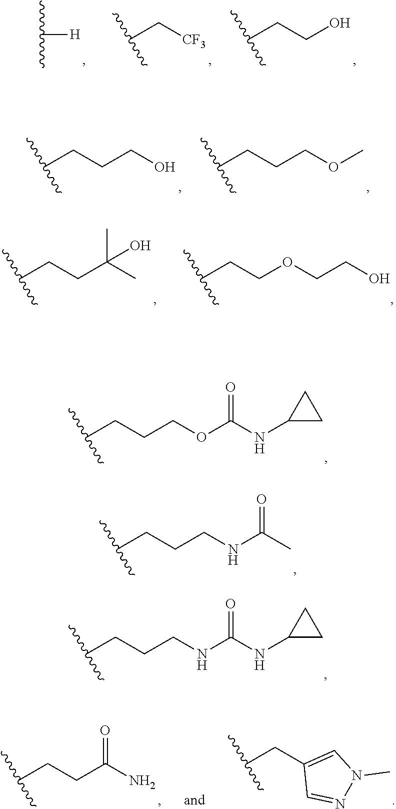

The definitions of the other variables are provided in the second through twenty-first embodiments or formula (I0).

In a twenty-third embodiment, the present disclosure provides a compound according to the any one of the second through twenty-second embodiments, a pharmaceutically acceptable salt, or a stereoisomer thereof, wherein R⁷ is halogen, cyano, C₁₋₄alkyl, 3-6 membered cycloalkyl, 3-6 membered heterocyclyl, phenyl, or 5-7 membered heteroaryl; wherein said C₁₋₄alkyl, 3-6 membered cycloalkyl, 3-6 membered heterocyclyl, phenyl, or 5-7 membered heteroaryl represented by R⁷ is optionally substituted with one or more halogen; and n is 0 or 1. The definitions of the other variables are provided in the second through twenty-second embodiments or formula (I0).

In a twenty-fourth embodiment, the present disclosure provides a compound according to the any one of the second through twenty-third embodiments, a pharmaceutically acceptable salt, or a stereoisomer thereof, wherein n is 0. The definitions of the other variables are provided in the second through twenty-third embodiments or formula (I0).

In a twenty-fifth embodiment, the present disclosure provides a compound according to the any one of the second through twenty-fourth embodiments, a pharmaceutically acceptable salt, or a stereoisomer thereof, wherein the compound is represented by Formula (II)

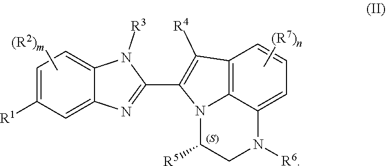

The definitions of the other variables are provided in the second through twenty-fourth embodiments or formula (I0).

In a twenty-sixth embodiment, the present disclosure provides a compound according to the any one of the second through twenty-fifth embodiments, a pharmaceutically acceptable salt, or a stereoisomer thereof, wherein R¹ is selected from

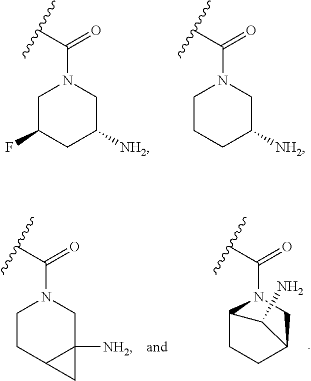

The definitions of the other variables are provided in the second through twenty-fifth embodiments or formula (I0). In one embodiment, R¹ is

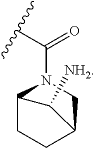

In a twenty-seventh embodiment, the present disclosure provides a compound according to the any one of the second through twenty-sixth embodiments, a pharmaceutically acceptable salt, or a stereoisomer thereof, wherein R^{a}, R^{b}, and R^{c} are each independently hydrogen or C₁₋₆alkyl. The definitions of the other variables are provided in the second through twenty-sixth embodiments or formula (I0).

In a twenty-eighth embodiment, the present disclosure provides a compound according to formula (I0), a pharmaceutically acceptable salt, or a stereoisomer thereof, wherein

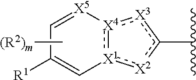

is selected from the group consisting of

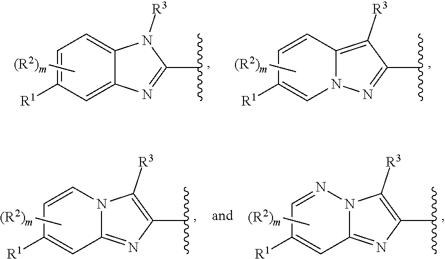

wherein the definition of each variable is defined in the first and the third through twenty-seventh embodiments.

In a twenty-ninth embodiment, the present disclosure provides a compound according to formula (I0), a pharmaceutically acceptable salt, or a stereoisomer thereof, wherein ring T is represented by Formula (T1) or (T3),

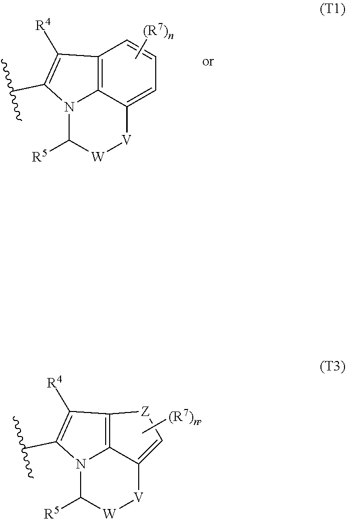

and the definitions of remaining variables are as defined in the first and the third through twenty-eighth embodiments. The definitions of the other variables are provided in the first embodiment.

In a thirtieth embodiment, the present disclosure provides a compound according to any one of the first, twenty-eighth, and twenty-ninth embodiments, a pharmaceutically acceptable salt, or a stereoisomer thereof, wherein W is —CH₂—, —CH(CH₃)—, or —C(═O)—, and the definitions of remaining variables are as defined in the first through fifth, the seventh through twenty-fourth, and the twenty-sixth through twenty-ninth embodiments.

In a thirty-first embodiment, the present disclosure provides a compound according to any one of the first and twenty-eighth through thirtieth embodiments, a pharmaceutically acceptable salt, or a stereoisomer thereof, wherein V is —C(═O)—, and the definitions of remaining variables are as defined in the first, the third through twenty-fourth, and the twenty-sixth through thirtieth embodiments.

In a thirty-second embodiment, the present disclosure provides a compound according to the first embodiment, a pharmaceutically acceptable salt, or a stereoisomer thereof, wherein the compound is represented by Formula (III),

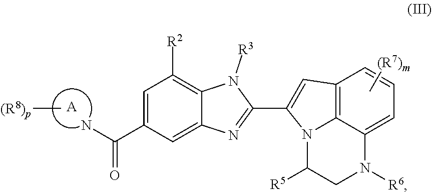

wherein
ring A is selected from the group consisting of

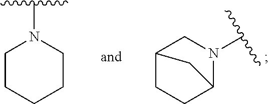 [SMILES: *N1CCCCC1 ; *N1CC2CCC1C2]

R² is halogen, CN, C₁₋₆alkyl, or C₁₋₆alkoxyl;
R³ is C₁₋₆alkyl, C₂₋₆alkynyl, —CH₂-3-5 membered cycloalkyl,
   —CH₂-3-5 membered heterocyclyl, —CH₂-phenyl, or —CH₂-5 membered heteroaryl; wherein said C₁₋₆alkyl, C₂₋₆alkynyl, cycloalkyl, heterocyclyl, phenyl, or heteroaryl represented by R³ or in the group represented by R³ is optionally substituted with one to three groups selected from halogen and C₁₋₆ alkyl;
R⁵ is hydrogen, C₁₋₃alkyl, or 3-4 membered cycloalkyl;
R⁶ is hydrogen or C₁₋₆alkyl; wherein said C₁₋₆alkyl represented by R⁶ is optionally substituted with one to three groups selected from halogen, hydroxyl, and C₁₋₆alkoxy;
R⁷ is halogen, cyano, C₁₋₆alkyl, haloC₁₋₆alkyl, or —S(═O)₂C₁₋₃alkyl;
R⁸ is halogen or NH₂;
p is 0, 1, or 2; and
n is 0 or 1.
   The definitions of the other variables are provided in the first embodiment. In one specific embodiment,

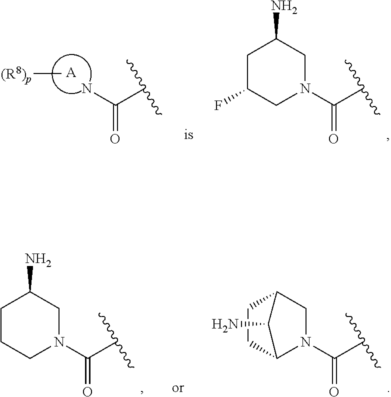

In a thirty-third embodiment, the present disclosure provides a compound according to the first embodiment, a pharmaceutically acceptable salt, or a stereoisomer thereof, wherein the compound is represented by Formula (IIIA),

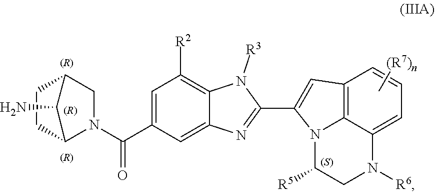

wherein
R² is halogen, CN, C₁₋₆alkyl, or C₁₋₆alkoxyl;
R³ is C₁₋₄alkyl;
R⁵ is hydrogen, C₁₋₃alkyl, or 3-4 membered cycloalkyl;
R⁶ is hydrogen or C₁₋₆alkyl; wherein said C₁₋₆alkyl represented by R⁶ is optionally substituted with one to three groups selected from halogen, hydroxyl, and methoxy;
R⁷ is halogen, cyano, C₁₋₆alkyl, haloC₁₋₆alkyl, or —S(═O)₂C₁₋₃alkyl; and
n is 0 or 1.
   The definitions of the other variables are provided in the first embodiment.

In a thirty-fourth embodiment, the present disclosure provides a compound according to the thirty-third embodiment, a pharmaceutically acceptable salt, or a stereoisomer thereof, wherein R² is fluoro; R³ is methyl; R⁵ is ethyl, isopropyl, or cyclopropyl; R⁶ is hydrogen or C₁₋₃alkyl; wherein said C₁₋₃alkyl represented by R⁶ is optionally substituted with hydroxyl; R⁷ is cyano or —S(═O)₂CH₃; and n is 0 or 1. The definitions of the other variables are provided in the thirty-third embodiment.

In a thirty-fifth embodiment, the present disclosure provides a compound according to the first embodiment, a pharmaceutically acceptable salt, or a stereoisomer thereof, wherein ring T is represented by Formula (T2) or (T4),

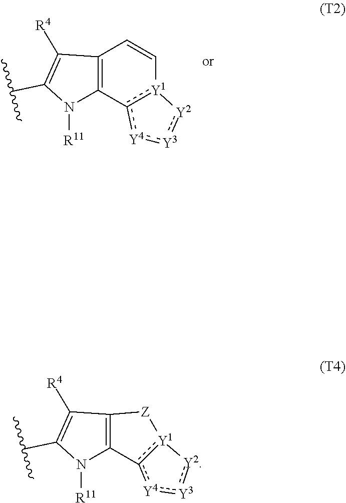

The definitions of the other variables are provided in the first embodiment.

In a thirty-sixth embodiment, the present disclosure provides a compound according to the first embodiment, a pharmaceutically acceptable salt, or a stereoisomer thereof, wherein the compound is represented by formula (IV),

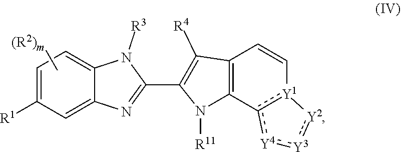

wherein
R¹ is

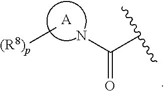

The definitions of the other variables are provided in the first embodiment.

In a thirty-seventh embodiment, the present disclosure provides a compound according to the thirty-sixth embodiment, a pharmaceutically acceptable salt, or a stereoisomer thereof, wherein ring A is 4-9 membered heterocyclyl. The definitions of the other variables are provided in the thirty-sixth embodiment.

In a thirty-eighth embodiment, the present disclosure provides a compound according to the thirty-sixth or the thirty-seventh embodiment, a pharmaceutically acceptable salt, or a stereoisomer thereof, wherein ring A is 4-6 membered monocyclic heterocyclyl or 6-8 membered bicyclic heterocyclyl. The definitions of the other variables are provided in the thirty-sixth or the thirty-seventh embodiment.

In a thirty-ninth embodiment, the present disclosure provides a compound according to any one of the thirty-sixth through the thirty-eighth embodiments, a pharmaceutically acceptable salt, or a stereoisomer thereof, wherein ring A is

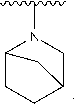 [SMILES: *N1CC2CCC1C2]

The definitions of the other variables are provided in the thirty-sixth through the thirty-eighth embodiments.

In a fortieth embodiment, the present disclosure provides a compound according to any one of the thirty-sixth through the thirty-ninth embodiments, a pharmaceutically acceptable salt, or a stereoisomer thereof, wherein R⁸ is halogen, C₁₋₆alkyl, haloC₁₋₆alkyl, —NR^{a}R^{b}, —NR^{a}(C═O)R^{b}, or —NR^{a}C(═O)OR^{b}; and p is 0, 1, 2, or 3. The definitions of the other variables are provided in the thirty-sixth through the thirty-ninth embodiments.

In a forty-first embodiment, the present disclosure provides a compound according to any one of the thirty-sixth through the fortieth embodiments, a pharmaceutically acceptable salt, or a stereoisomer thereof, wherein R⁸ is NH₂; and p is 1. The definitions of the other variables are provided in the thirty-sixth through the fortieth embodiments.

In a forty-second embodiment, the present disclosure provides a compound according to any one of the thirty-sixth through the forty-first embodiments, a pharmaceutically acceptable salt, or a stereoisomer thereof, wherein R² is halogen, CN, C₁₋₆alkyl, or C₁₋₆alkoxyl; and m is 0, 1, or 2. The definitions of the other variables are provided in the thirty-sixth through the forty-first embodiments.

In a forty-third embodiment, the present disclosure provides a compound according to any one of the thirty-sixth through the forty-second embodiments, a pharmaceutically acceptable salt, or a stereoisomer thereof, wherein R² is —F; and m is 1. The definitions of the other variables are provided in the thirty-sixth through the forty-second embodiments.

In a forty-fourth embodiment, the present disclosure provides a compound according to any one of the thirty-sixth through the forty-third embodiments, a pharmaceutically acceptable salt, or a stereoisomer thereof, wherein R³ is C₁₋₄alkyl, C₁₋₄alkoxyl, C₂₋₄alkynyl, —CH₂-3-5 membered cycloalkyl, —CH₂-3-5 membered heterocyclyl, —CH₂-phenyl, or —CH₂-5-6 membered heteroaryl; wherein said C₁₋₄alkyl, C₁₋₄alkoxyl, C₁₋₄alkynyl, cycloalkyl, heterocyclyl, phenyl, or heteroaryl represented by R³ or in the group represented by R³ is optionally substituted with one to three groups selected from halogen, C₁₋₄ alkyl, hydroxyl, and C₁₋₄alkoxyl. The definitions of the other variables are provided in the thirty-sixth through the forty-third embodiments.

In a forty-fifth embodiment, the present disclosure provides a compound according to any one of the thirty-sixth through the forty-fourth embodiments, a pharmaceutically acceptable salt, or a stereoisomer thereof, wherein R³ is C₁₋₂alkyl, C₂₋₃alkynyl, —CH₂-3-4 membered cycloalkyl, —CH₂-3-4 membered heterocyclyl, —CH₂-phenyl, or —CH₂-5 membered heteroaryl; wherein said C₁₋₂alkyl, C₁₋₂alkoxyl, C₂₋₃alkynyl, cycloalkyl, heterocyclyl, phenyl, or heteroaryl represented by R³ or in the group represented by R³ is optionally substituted with one to three groups selected from halogen, C₁₋₂ alkyl, and C₁₋₂alkoxyl. The definitions of the other variables are provided in the thirty-sixth through the forty-fourth embodiments.

In a forty-sixth embodiment, the present disclosure provides a compound according to any one of the thirty-sixth through the forty-fifth embodiments, a pharmaceutically acceptable salt, or a stereoisomer thereof, wherein R³ is —CH₃. The definitions of the other variables are provided in the thirty-sixth through the forty-fifth embodiments.

In a forty-seventh embodiment, the present disclosure provides a compound according to any one of the thirty-sixth through the forty-sixth embodiments, a pharmaceutically acceptable salt, or a stereoisomer thereof, wherein R⁴ is hydrogen. The definitions of the other variables are provided in the thirty-sixth through the forty-sixth embodiments.

In a forty-eighth embodiment, the present disclosure provides a compound according to any one of the thirty-sixth through the forty-seventh embodiments, a pharmaceutically acceptable salt, or a stereoisomer thereof, wherein R¹¹ is —CH₂-3-6 membered cycloalkyl. The definitions of the other variables are provided in the thirty-sixth through the forty-seventh embodiments.

In a forty-ninth embodiment, the present disclosure provides a compound according to any one of the thirty-sixth through the forty-eighth embodiments, a pharmaceutically acceptable salt, or a stereoisomer thereof, wherein R¹¹ is or

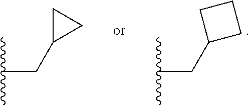 [SMILES: *CC1CC1 ; *CC1CCC1]

The definitions of the other variables are provided in the thirty-sixth through the forty-eighth embodiments.

In a fiftieth embodiment, the present disclosure provides a compound according to any one of the thirty-sixth through the forty-ninth embodiments, a pharmaceutically acceptable salt, or a stereoisomer thereof, wherein

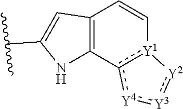

is selected from the group consisting of

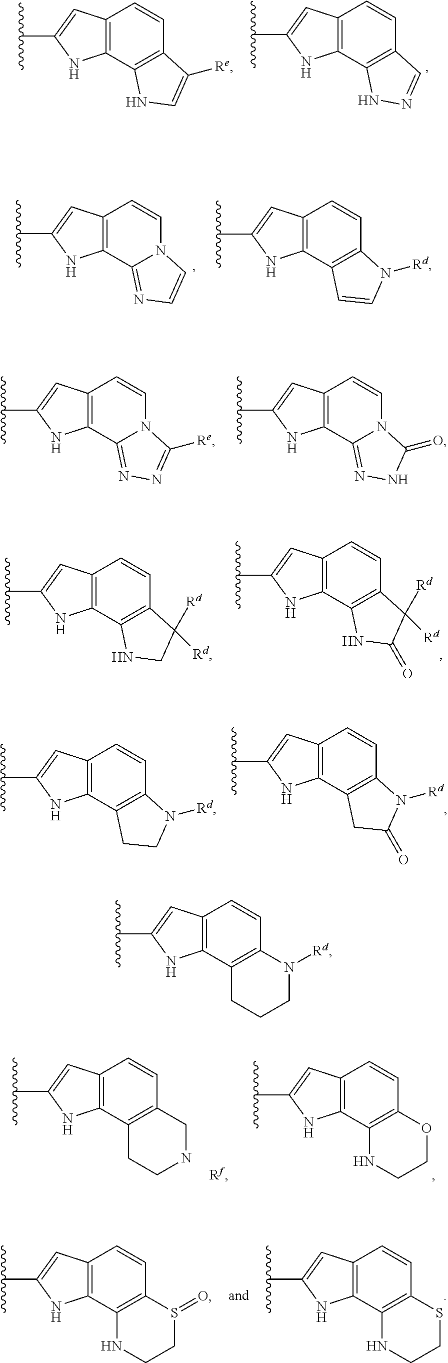

The definitions of the other variables are provided in the thirty-sixth through the forty-ninth embodiments.

In a fifty-first embodiment, the present disclosure provides a compound according to any one of the thirty-sixth through the fiftieth embodiments, a pharmaceutically acceptable salt, or a stereoisomer thereof, wherein
R^{d} is hydrogen or C₁₋₄alkyl;
R^{e} is hydrogen, halogen, or C₁₋₄alkyl; and
R^{f} is hydrogen, C₁₋₄alkyl, —C(═O)C₁₋₄alkyl, or 3-5 membered cycloalkyl.
   The definitions of the other variables are provided in the thirty-sixth through the fiftieth embodiments.

In a fifty-second embodiment, the present disclosure provides a compound according to the thirty-sixth embodiment, a pharmaceutically acceptable salt, or a stereoisomer thereof, wherein the compound is represented by formula (V),

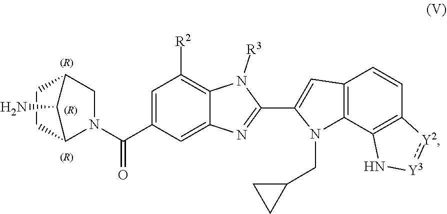

wherein
R² is halogen, CN, C₁₋₆alkyl, or C₁₋₆alkoxyl;
R³ is C₁₋₄alkyl;
Y² is —S—, —O—, —N(R^{d})—, —CH₂—, or —CH═; and
Y³ is —CH₂—, —HC═, —N═, or —CH₂—CH₂—.
   The definitions of the other variables are provided in the thirty-sixth embodiment.

In a fifty-third embodiment, the present disclosure provides a compound according to the fifty-second embodiment, a pharmaceutically acceptable salt, or a stereoisomer thereof, wherein R² is fluoro; and R³ is methyl. The definitions of the other variables are provided in the fifty-second embodiment.

In a fifty-four embodiment, the present disclosure provides a compound according to the thirty-fifth or thirty-sixth embodiment, a pharmaceutically acceptable salt, or a stereoisomer thereof, wherein R¹ is

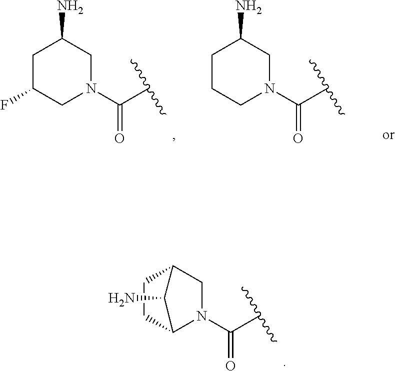 [SMILES: *C(=O)N1C[C@H](N)C[C@@H](F)C1 ; *C(=O)N1CCC[C@@H](N)C1 ; *C(=O)N1C[C@H]2CC[C@@H]1[C@@H]2N]

In one embodiment, the present disclosure provides a compound selected from the compounds disclosed in examples and Table 1, a pharmaceutically acceptable salt or a stereoisomer thereof.

TABLE 1

| Example | |
|---|---|
| No. | Structure |
| 1 | 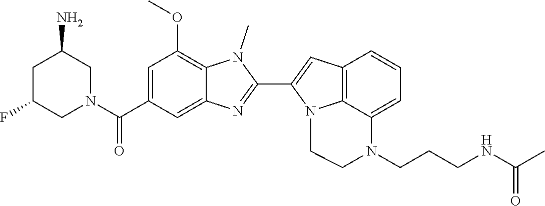 [SMILES: COc1cc(C(=O)N2C[C@H](N)C[C@@H](F)C2)cc2nc(-c3cc4cccc5c4n3CCN5CCCNC(C)=O)n(C)c12] |
| 2 | 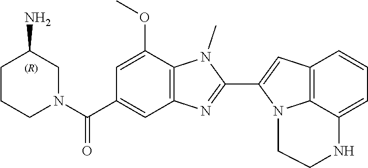 |
| 3 | 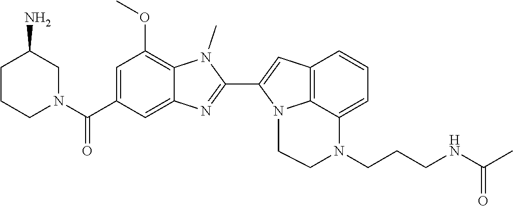 [SMILES: COc1cc(C(=O)N2CCC[C@@H](N)C2)cc2nc(-c3cc4cccc5c4n3CCN5CCCNC(C)=O)n(C)c12] |
| 4 | 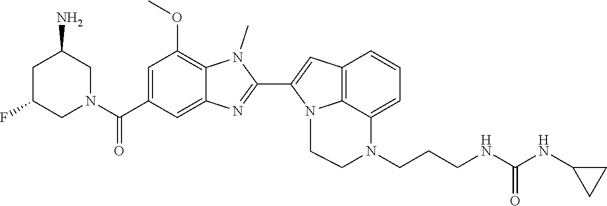 [SMILES: COc1cc(C(=O)N2C[C@H](N)C[C@@H](F)C2)cc2nc(-c3cc4cccc5c4n3CCN5CCCNC(=O)NC3CC3)n(C)c12] |
| 5 | 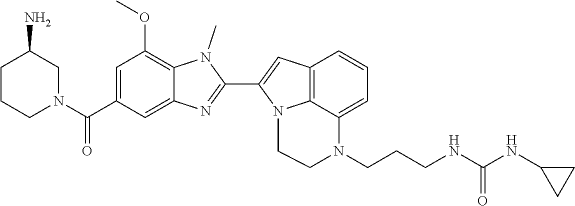 [SMILES: COc1cc(C(=O)N2CCC[C@@H](N)C2)cc2nc(-c3cc4cccc5c4n3CCN5CCCNC(=O)NC3CC3)n(C)c12] |
| 6 | 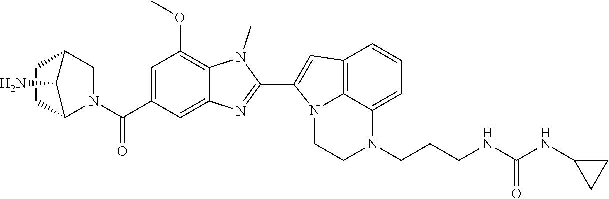 [SMILES: COc1cc(C(=O)N2C[C@H]3CC[C@@H]2[C@@H]3N)cc2nc(-c3cc4cccc5c4n3CCN5CCCNC(=O)NC3CC3)n(C)c12] |
| 55 | 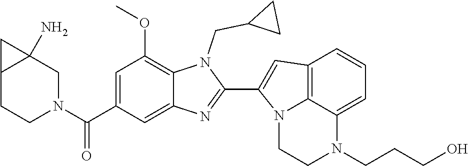 [SMILES: COc1cc(C(=O)N2CCC3CC3(N)C2)cc2nc(-c3cc4cccc5c4n3CCN5CCCO)n(CC3CC3)c12] |
| 56 | 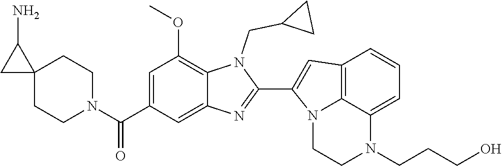 [SMILES: COc1cc(C(=O)N2CCC3(CC2)CC3N)cc2nc(-c3cc4cccc5c4n3CCN5CCCO)n(CC3CC3)c12] |
| 57 | 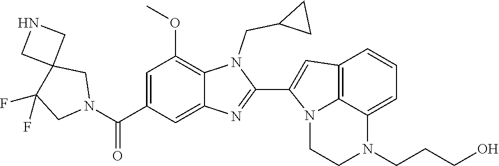 [SMILES: COc1cc(C(=O)N2CC(F)(F)C3(CNC3)C2)cc2nc(-c3cc4cccc5c4n3CCN5CCCO)n(CC3CC3)c12] |
| 58 | 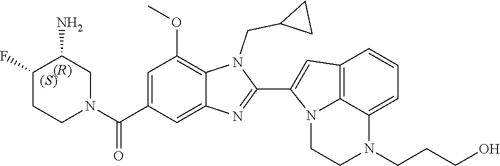 |
| 59 | 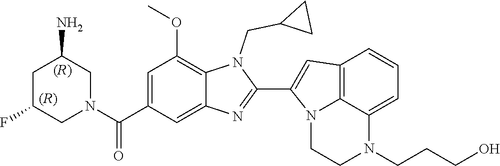 |
| 60 | 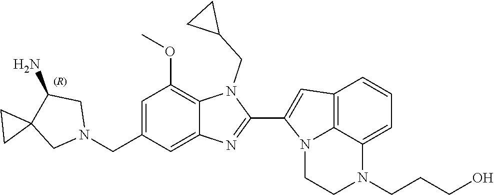 |
| 7 | 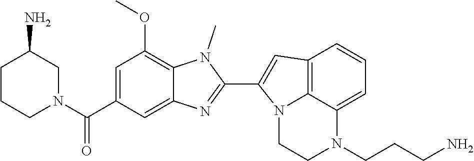 [SMILES: COc1cc(C(=O)N2CCC[C@@H](N)C2)cc2nc(-c3cc4cccc5c4n3CCN5CCCN)n(C)c12] |
| 8 | 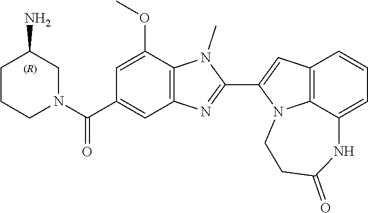 |
| 9 | 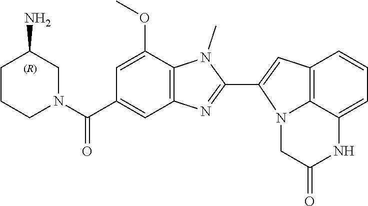 |
| 10 | 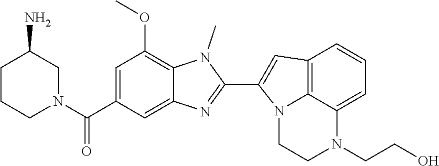 [SMILES: COc1cc(C(=O)N2CCC[C@@H](N)C2)cc2nc(-c3cc4cccc5c4n3CCN5CCO)n(C)c12] |
| 11 | 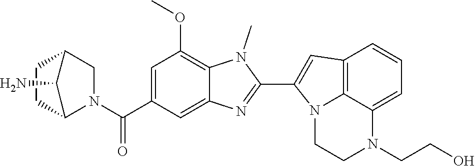 [SMILES: COc1cc(C(=O)N2C[C@H]3CC[C@@H]2[C@@H]3N)cc2nc(-c3cc4cccc5c4n3CCN5CCO)n(C)c12] |
| 12 | 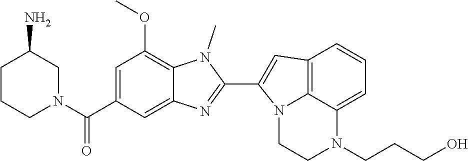 [SMILES: COc1cc(C(=O)N2CCC[C@@H](N)C2)cc2nc(-c3cc4cccc5c4n3CCN5CCCO)n(C)c12] |
| 13 | 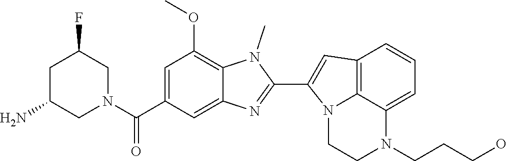 [SMILES: COc1cc(C(=O)N2C[C@H](N)C[C@@H](F)C2)cc2nc(-c3cc4cccc5c4n3CCN5CCCO)n(C)c12] |
| 14 | 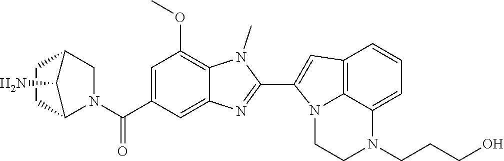 [SMILES: COc1cc(C(=O)N2C[C@H]3CC[C@@H]2[C@@H]3N)cc2nc(-c3cc4cccc5c4n3CCN5CCCO)n(C)c12] |
| 15 | 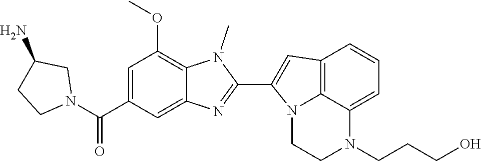 [SMILES: COc1cc(C(=O)N2CC[C@@H](N)C2)cc2nc(-c3cc4cccc5c4n3CCN5CCCO)n(C)c12] |
| 16 | 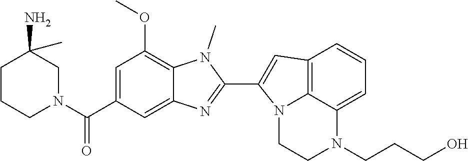 [SMILES: COc1cc(C(=O)N2CCC[C@@](C)(N)C2)cc2nc(-c3cc4cccc5c4n3CCN5CCCO)n(C)c12] |
| 61 | 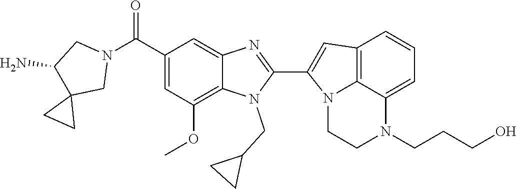 [SMILES: COc1cc(C(=O)N2C[C@@H](N)C3(CC3)C2)cc2nc(-c3cc4cccc5c4n3CCN5CCCO)n(CC3CC3)c12] |
| 62 | 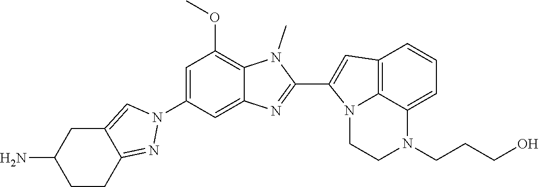 [SMILES: COc1cc(-n2cc3c(n2)CCC(N)C3)cc2nc(-c3cc4cccc5c4n3CCN5CCCO)n(C)c12] |
| 63 | 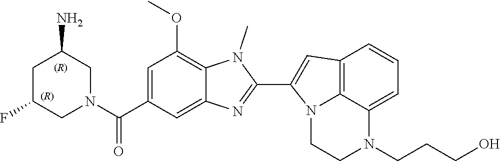 |
| 64 | 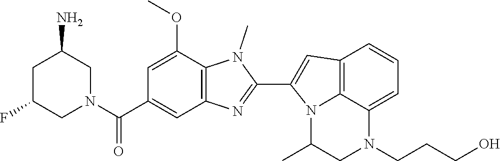 [SMILES: COc1cc(C(=O)N2C[C@H](N)C[C@@H](F)C2)cc2nc(-c3cc4cccc5c4n3C(C)CN5CCCO)n(C)c12] |
| 65 | 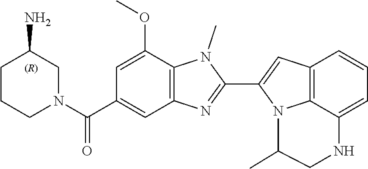 |
| 66 | 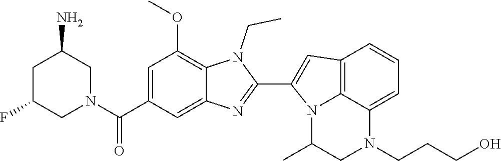 [SMILES: CCn1c(-c2cc3cccc4c3n2C(C)CN4CCCO)nc2cc(C(=O)N3C[C@H](N)C[C@@H](F)C3)cc(OC)c21] |
| 67 | 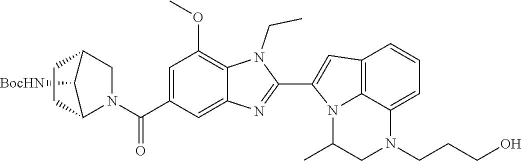 [SMILES: CCn1c(-c2cc3cccc4c3n2C(C)CN4CCCO)nc2cc(C(=O)N3C[C@H]4CC[C@@H]3[C@@H]4NC(=O)OC(C)(C)C)cc(OC)c21] |
| 68 | 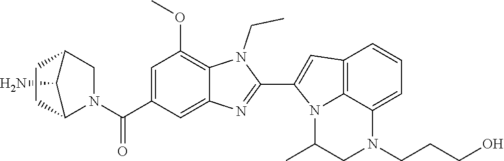 [SMILES: CCn1c(-c2cc3cccc4c3n2C(C)CN4CCCO)nc2cc(C(=O)N3C[C@H]4CC[C@@H]3[C@@H]4N)cc(OC)c21] |
| 69 | 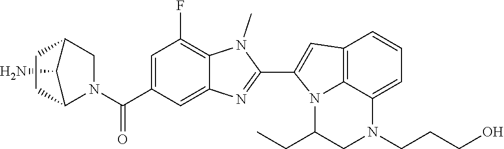 [SMILES: CCC1CN(CCCO)c2cccc3cc(-c4nc5cc(C(=O)N6C[C@H]7CC[C@@H]6[C@@H]7N)cc(F)c5n4C)n1c23] |
| 70 | 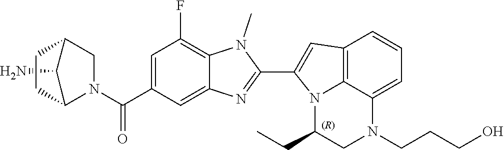 |
| 17 | 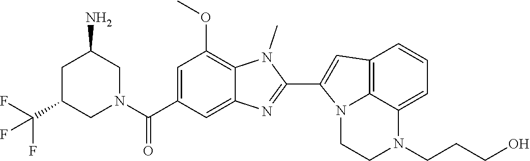 [SMILES: COc1cc(C(=O)N2C[C@H](N)C[C@@H](C(F)(F)F)C2)cc2nc(-c3cc4cccc5c4n3CCN5CCCO)n(C)c12] |
| 18 | 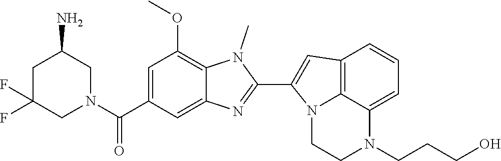 [SMILES: COc1cc(C(=O)N2C[C@H](N)CC(F)(F)C2)cc2nc(-c3cc4cccc5c4n3CCN5CCCO)n(C)c12] |
| 19 | 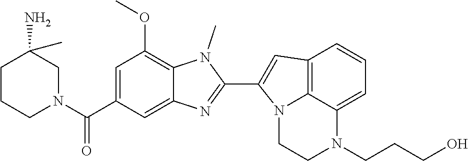 [SMILES: COc1cc(C(=O)N2CCC[C@](C)(N)C2)cc2nc(-c3cc4cccc5c4n3CCN5CCCO)n(C)c12] |
| 20 | 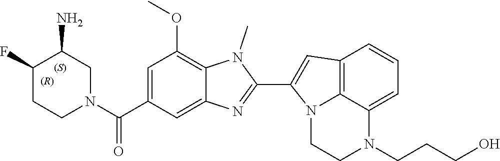 |
| 21 | 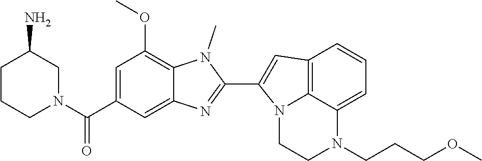 [SMILES: COCCCN1CCn2c(-c3nc4cc(C(=O)N5CCC[C@@H](N)C5)cc(OC)c4n3C)cc3cccc1c32] |
| 22 | 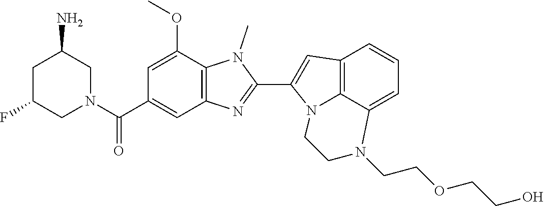 [SMILES: COc1cc(C(=O)N2C[C@H](N)C[C@@H](F)C2)cc2nc(-c3cc4cccc5c4n3CCN5CCOCCO)n(C)c12] |
| 23 | 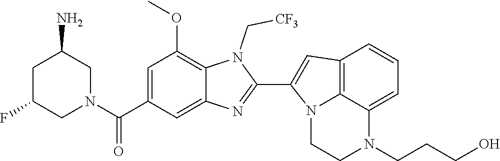 [SMILES: COc1cc(C(=O)N2C[C@H](N)C[C@@H](F)C2)cc2nc(-c3cc4cccc5c4n3CCN5CCCO)n(CC(F)(F)F)c12] |
| 24 | 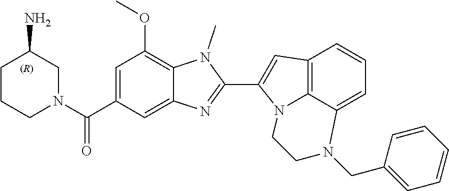 |
| 25 | 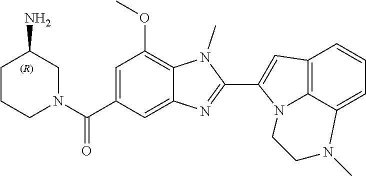 |
| 26 | 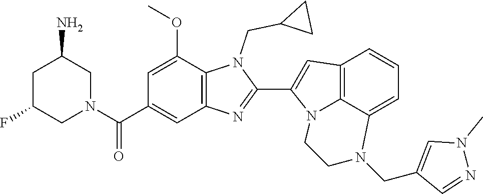 [SMILES: COc1cc(C(=O)N2C[C@H](N)C[C@@H](F)C2)cc2nc(-c3cc4cccc5c4n3CCN5Cc3cnn(C)c3)n(CC3CC3)c12] |
| 71 | 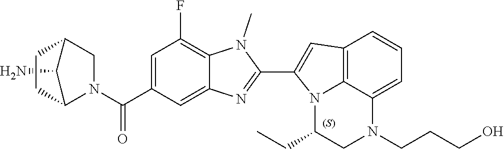 |
| 72 | 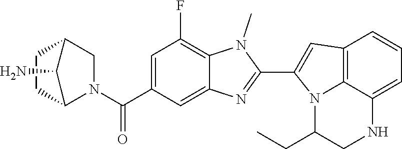 [SMILES: CCC1CNc2cccc3cc(-c4nc5cc(C(=O)N6C[C@H]7CC[C@@H]6[C@@H]7N)cc(F)c5n4C)n1c23] |
| 73 | 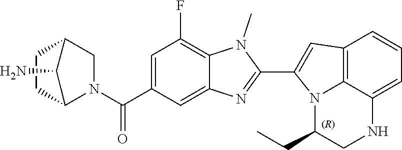 |
| 74 | 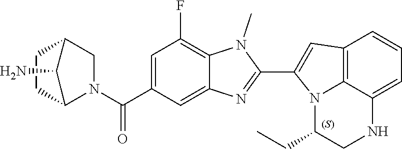 |
| 75 | 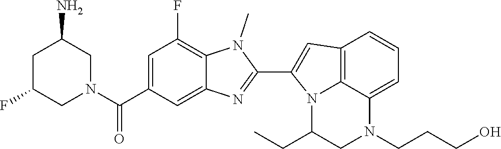 [SMILES: CCC1CN(CCCO)c2cccc3cc(-c4nc5cc(C(=O)N6C[C@H](N)C[C@@H](F)C6)cc(F)c5n4C)n1c23] |
| 76 | 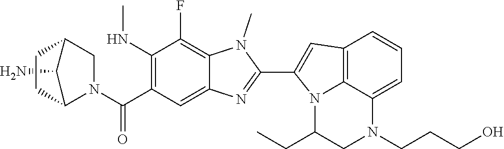 [SMILES: CCC1CN(CCCO)c2cccc3cc(-c4nc5cc(C(=O)N6C[C@H]7CC[C@@H]6[C@@H]7N)c(NC)c(F)c5n4C)n1c23] |
| 77 | 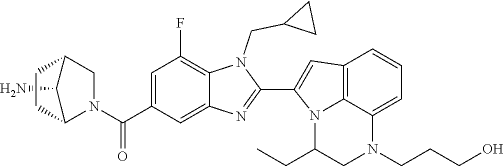 [SMILES: CCC1CN(CCCO)c2cccc3cc(-c4nc5cc(C(=O)N6C[C@H]7CC[C@@H]6[C@@H]7N)cc(F)c5n4CC4CC4)n1c23] |
| 78 | 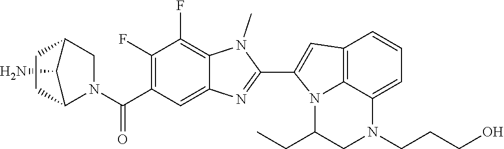 [SMILES: CCC1CN(CCCO)c2cccc3cc(-c4nc5cc(C(=O)N6C[C@H]7CC[C@@H]6[C@@H]7N)c(F)c(F)c5n4C)n1c23] |
| 79 | 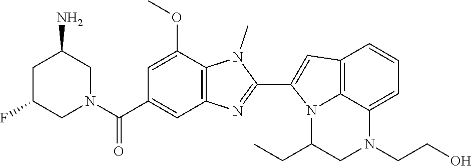 [SMILES: CCC1CN(CCO)c2cccc3cc(-c4nc5cc(C(=O)N6C[C@H](N)C[C@@H](F)C6)cc(OC)c5n4C)n1c23] |
| 80 | 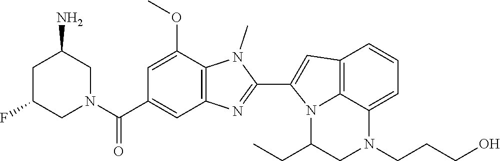 [SMILES: CCC1CN(CCCO)c2cccc3cc(-c4nc5cc(C(=O)N6C[C@H](N)C[C@@H](F)C6)cc(OC)c5n4C)n1c23] |
| 27 | 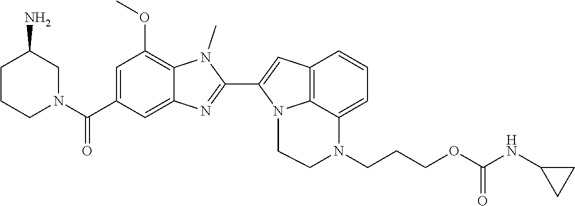 [SMILES: COc1cc(C(=O)N2CCC[C@@H](N)C2)cc2nc(-c3cc4cccc5c4n3CCN5CCCOC(=O)NC3CC3)n(C)c12] |
| 28 | 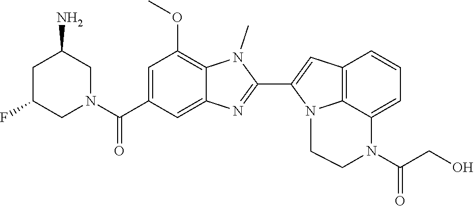 [SMILES: COc1cc(C(=O)N2C[C@H](N)C[C@@H](F)C2)cc2nc(-c3cc4cccc5c4n3CCN5C(=O)CO)n(C)c12] |
| 29 | 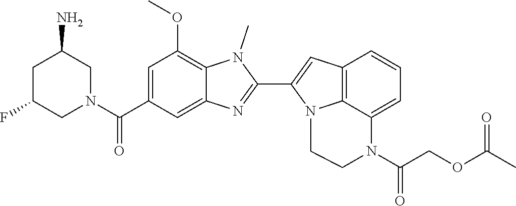 [SMILES: COc1cc(C(=O)N2C[C@H](N)C[C@@H](F)C2)cc2nc(-c3cc4cccc5c4n3CCN5C(=O)COC(C)=O)n(C)c12] |
| 30 | 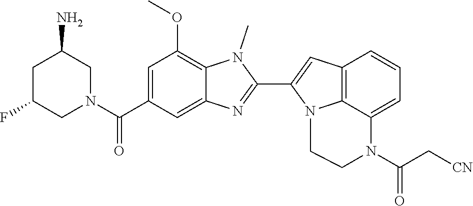 [SMILES: COc1cc(C(=O)N2C[C@H](N)C[C@@H](F)C2)cc2nc(-c3cc4cccc5c4n3CCN5C(=O)CC#N)n(C)c12] |
| 31 | 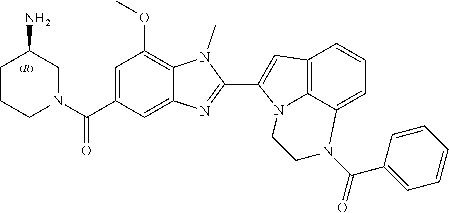 |
| 32 | 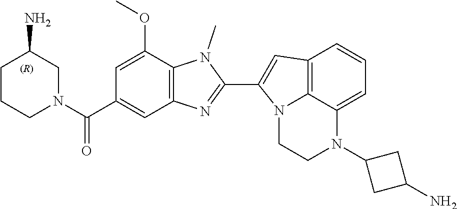 |
| 33 | 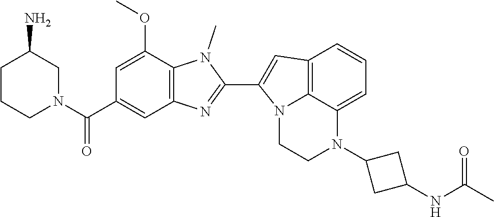 [SMILES: COc1cc(C(=O)N2CCC[C@@H](N)C2)cc2nc(-c3cc4cccc5c4n3CCN5C3CC(NC(C)=O)C3)n(C)c12] |
| 34 | 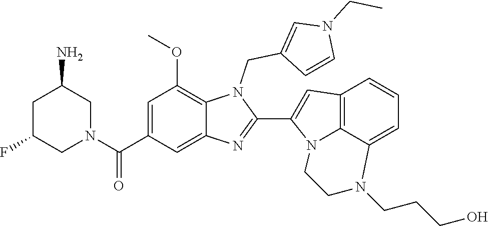 [SMILES: CCn1ccc(Cn2c(-c3cc4cccc5c4n3CCN5CCCO)nc3cc(C(=O)N4C[C@H](N)C[C@@H](F)C4)cc(OC)c32)c1] |
| 35 | 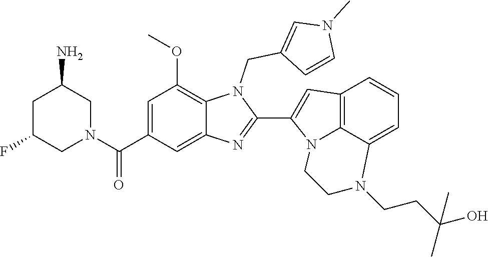 [SMILES: COc1cc(C(=O)N2C[C@H](N)C[C@@H](F)C2)cc2nc(-c3cc4cccc5c4n3CCN5CCC(C)(C)O)n(Cc3ccn(C)c3)c12] |
| 81 | 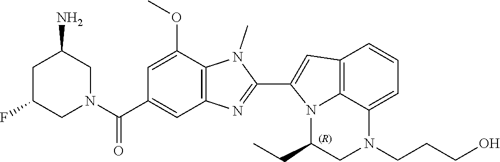 |
| 82 | 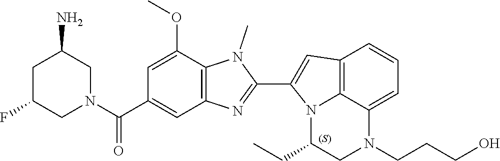 |
| 83 | 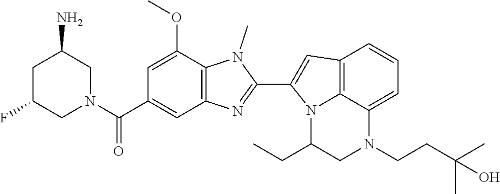 [SMILES: CCC1CN(CCC(C)(C)O)c2cccc3cc(-c4nc5cc(C(=O)N6C[C@H](N)C[C@@H](F)C6)cc(OC)c5n4C)n1c23] |
| 84 | 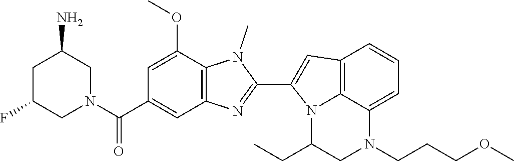 [SMILES: CCC1CN(CCCOC)c2cccc3cc(-c4nc5cc(C(=O)N6C[C@H](N)C[C@@H](F)C6)cc(OC)c5n4C)n1c23] |
| 85 | 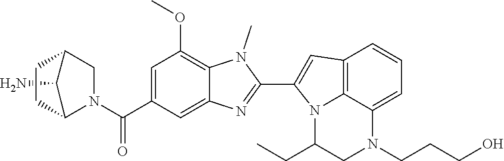 [SMILES: CCC1CN(CCCO)c2cccc3cc(-c4nc5cc(C(=O)N6C[C@H]7CC[C@@H]6[C@@H]7N)cc(OC)c5n4C)n1c23] |
| 86 | 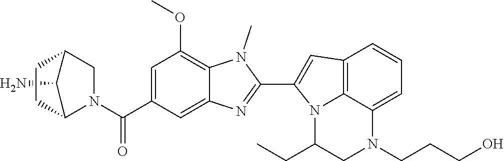 [SMILES: CCC1CN(CCCO)c2cccc3cc(-c4nc5cc(C(=O)N6C[C@H]7CC[C@@H]6[C@@H]7N)cc(OC)c5n4C)n1c23] |
| 87 | 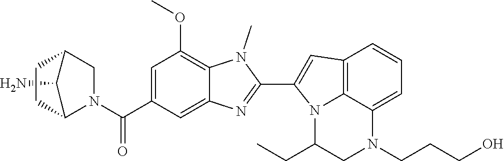 [SMILES: CCC1CN(CCCO)c2cccc3cc(-c4nc5cc(C(=O)N6C[C@H]7CC[C@@H]6[C@@H]7N)cc(OC)c5n4C)n1c23] |
| 88 | 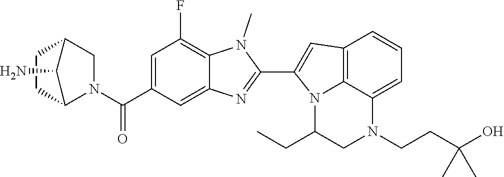 [SMILES: CCC1CN(CCC(C)(C)O)c2cccc3cc(-c4nc5cc(C(=O)N6C[C@H]7CC[C@@H]6[C@@H]7N)cc(F)c5n4C)n1c23] |
| 89 | 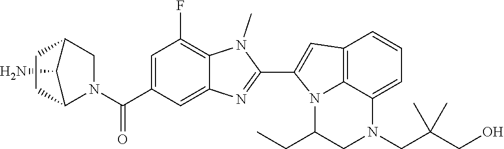 [SMILES: CCC1CN(CC(C)(C)CO)c2cccc3cc(-c4nc5cc(C(=O)N6C[C@H]7CC[C@@H]6[C@@H]7N)cc(F)c5n4C)n1c23] |
| 36 | 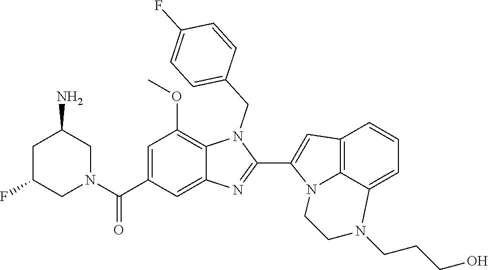 [SMILES: COc1cc(C(=O)N2C[C@H](N)C[C@@H](F)C2)cc2nc(-c3cc4cccc5c4n3CCN5CCCO)n(Cc3ccc(F)cc3)c12] |
| 37 | 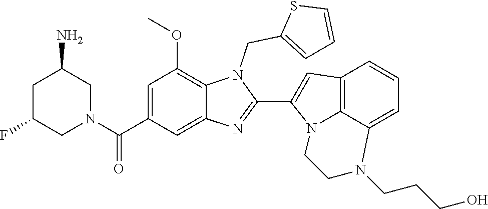 [SMILES: COc1cc(C(=O)N2C[C@H](N)C[C@@H](F)C2)cc2nc(-c3cc4cccc5c4n3CCN5CCCO)n(Cc3cccs3)c12] |
| 38 | 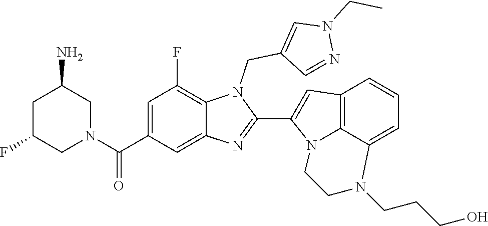 [SMILES: CCn1cc(Cn2c(-c3cc4cccc5c4n3CCN5CCCO)nc3cc(C(=O)N4C[C@H](N)C[C@@H](F)C4)cc(F)c32)cn1] |
| 39 | 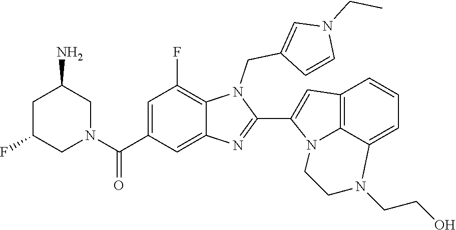 [SMILES: CCn1ccc(Cn2c(-c3cc4cccc5c4n3CCN5CCO)nc3cc(C(=O)N4C[C@H](N)C[C@@H](F)C4)cc(F)c32)c1] |
| 40 | 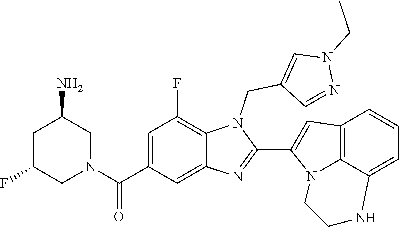 [SMILES: CCn1cc(Cn2c(-c3cc4cccc5c4n3CCN5)nc3cc(C(=O)N4C[C@H](N)C[C@@H](F)C4)cc(F)c32)cn1] |
| 41 | 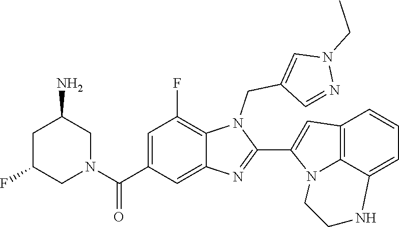 [SMILES: CCn1cc(Cn2c(-c3cc4cccc5c4n3CCN5)nc3cc(C(=O)N4C[C@H](N)C[C@@H](F)C4)cc(F)c32)cn1] |
| 42 | 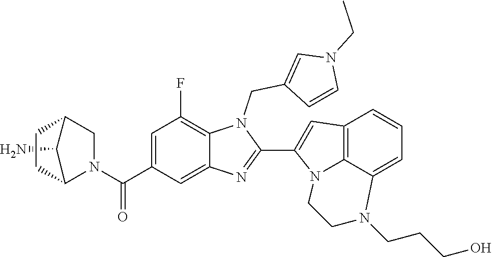 [SMILES: CCn1ccc(Cn2c(-c3cc4cccc5c4n3CCN5CCCO)nc3cc(C(=O)N4C[C@H]5CC[C@@H]4[C@@H]5N)cc(F)c32)c1] |
| 43 | 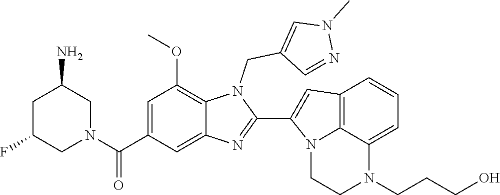 [SMILES: COc1cc(C(=O)N2C[C@H](N)C[C@@H](F)C2)cc2nc(-c3cc4cccc5c4n3CCN5CCCO)n(Cc3cnn(C)c3)c12] |
| 44 | 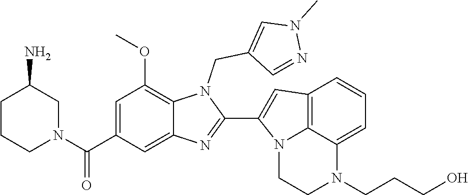 [SMILES: COc1cc(C(=O)N2CCC[C@@H](N)C2)cc2nc(-c3cc4cccc5c4n3CCN5CCCO)n(Cc3cnn(C)c3)c12] |
| 90 | 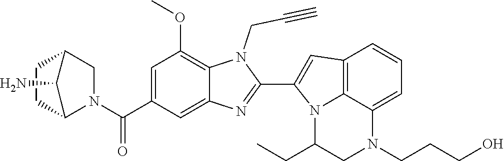 [SMILES: C#CCn1c(-c2cc3cccc4c3n2C(CC)CN4CCCO)nc2cc(C(=O)N3C[C@H]4CC[C@@H]3[C@@H]4N)cc(OC)c21] |
| 91 | 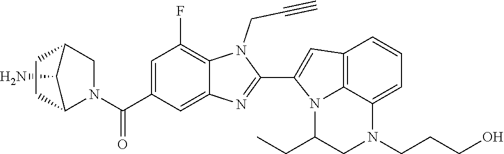 [SMILES: C#CCn1c(-c2cc3cccc4c3n2C(CC)CN4CCCO)nc2cc(C(=O)N3C[C@H]4CC[C@@H]3[C@@H]4N)cc(F)c21] |
| 92 | 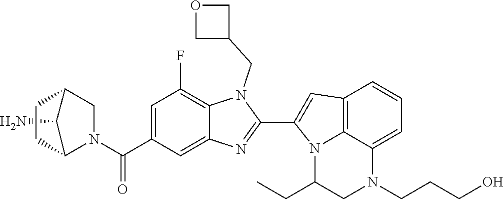 [SMILES: CCC1CN(CCCO)c2cccc3cc(-c4nc5cc(C(=O)N6C[C@H]7CC[C@@H]6[C@@H]7N)cc(F)c5n4CC4COC4)n1c23] |
| 93 | 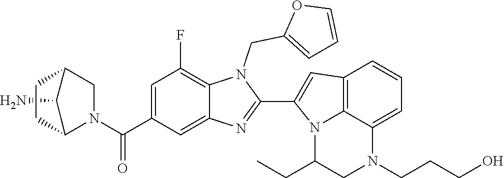 [SMILES: CCC1CN(CCCO)c2cccc3cc(-c4nc5cc(C(=O)N6C[C@H]7CC[C@@H]6[C@@H]7N)cc(F)c5n4Cc4ccco4)n1c23] |
| 94 | 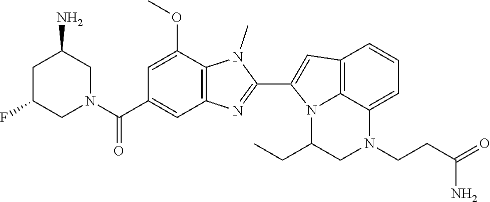 [SMILES: CCC1CN(CCC(N)=O)c2cccc3cc(-c4nc5cc(C(=O)N6C[C@H](N)C[C@@H](F)C6)cc(OC)c5n4C)n1c23] |
| 95 | 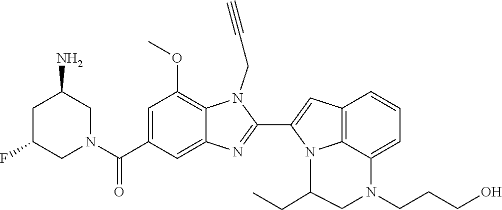 [SMILES: C#CCn1c(-c2cc3cccc4c3n2C(CC)CN4CCCO)nc2cc(C(=O)N3C[C@H](N)C[C@@H](F)C3)cc(OC)c21] |
| 96 | 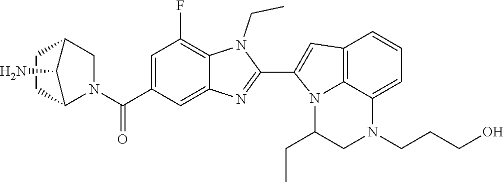 [SMILES: CCC1CN(CCCO)c2cccc3cc(-c4nc5cc(C(=O)N6C[C@H]7CC[C@@H]6[C@@H]7N)cc(F)c5n4CC)n1c23] |
| 97 | 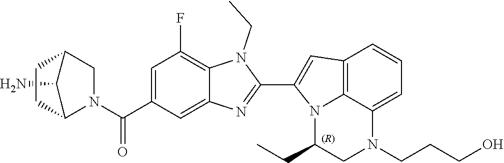 |
| 98 | 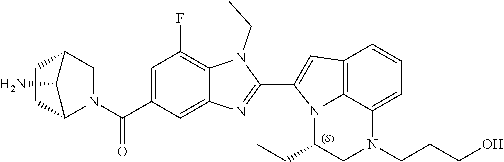 |
| 45 | 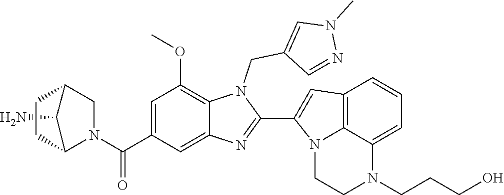 [SMILES: COc1cc(C(=O)N2C[C@H]3CC[C@@H]2[C@@H]3N)cc2nc(-c3cc4cccc5c4n3CCN5CCCO)n(Cc3cnn(C)c3)c12] |
| 46 | 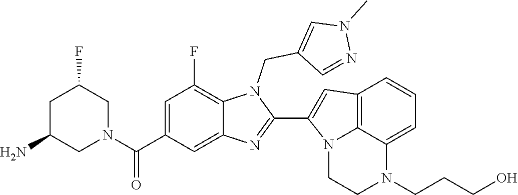 [SMILES: Cn1cc(Cn2c(-c3cc4cccc5c4n3CCN5CCCO)nc3cc(C(=O)N4C[C@@H](N)C[C@H](F)C4)cc(F)c32)cn1] |
| 47 | 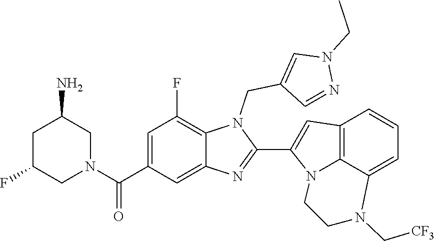 [SMILES: CCn1cc(Cn2c(-c3cc4cccc5c4n3CCN5CC(F)(F)F)nc3cc(C(=O)N4C[C@H](N)C[C@@H](F)C4)cc(F)c32)cn1] |
| 48 | 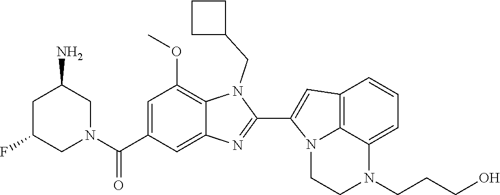 [SMILES: COc1cc(C(=O)N2C[C@H](N)C[C@@H](F)C2)cc2nc(-c3cc4cccc5c4n3CCN5CCCO)n(CC3CCC3)c12] |
| 49 | 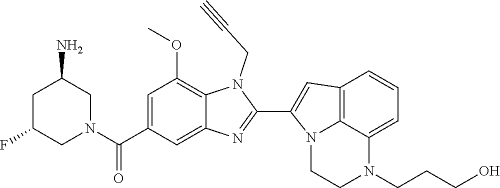 [SMILES: C#CCn1c(-c2cc3cccc4c3n2CCN4CCCO)nc2cc(C(=O)N3C[C@H](N)C[C@@H](F)C3)cc(OC)c21] |
| 50 | 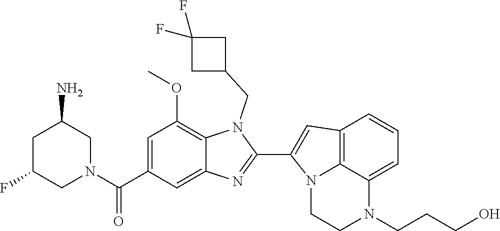 [SMILES: COc1cc(C(=O)N2C[C@H](N)C[C@@H](F)C2)cc2nc(-c3cc4cccc5c4n3CCN5CCCO)n(CC3CC(F)(F)C3)c12] |
| 51 | 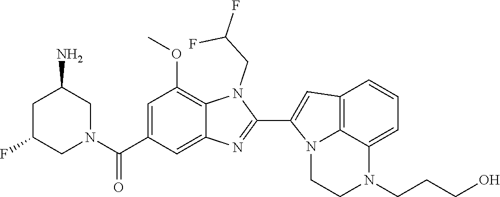 [SMILES: COc1cc(C(=O)N2C[C@H](N)C[C@@H](F)C2)cc2nc(-c3cc4cccc5c4n3CCN5CCCO)n(CC(F)F)c12] |
| 52 | 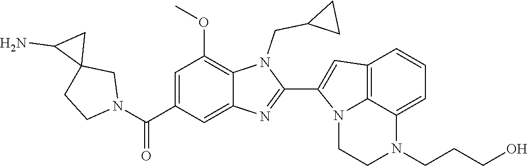 [SMILES: COc1cc(C(=O)N2CCC3(CC3N)C2)cc2nc(-c3cc4cccc5c4n3CCN5CCCO)n(CC3CC3)c12] |
| 53 | 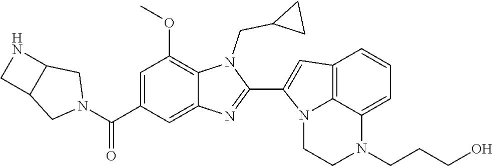 [SMILES: COc1cc(C(=O)N2CC3CNC3C2)cc2nc(-c3cc4cccc5c4n3CCN5CCCO)n(CC3CC3)c12] |
| 99 | 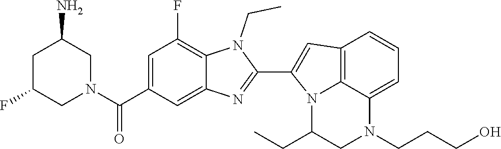 [SMILES: CCC1CN(CCCO)c2cccc3cc(-c4nc5cc(C(=O)N6C[C@H](N)C[C@@H](F)C6)cc(F)c5n4CC)n1c23] |
| 100 | 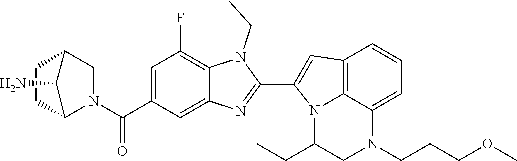 [SMILES: CCC1CN(CCCOC)c2cccc3cc(-c4nc5cc(C(=O)N6C[C@H]7CC[C@@H]6[C@@H]7N)cc(F)c5n4CC)n1c23] |
| 101 | 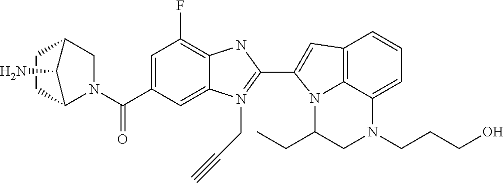 |
| 102 | 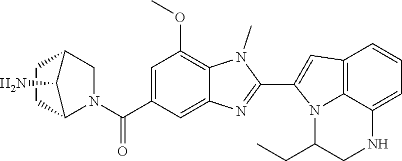 [SMILES: CCC1CNc2cccc3cc(-c4nc5cc(C(=O)N6C[C@H]7CC[C@@H]6[C@@H]7N)cc(OC)c5n4C)n1c23] |
| 103 | 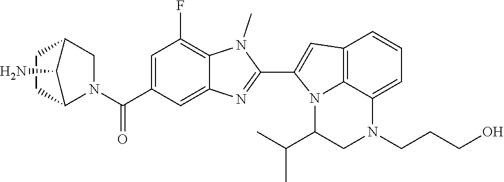 [SMILES: CC(C)C1CN(CCCO)c2cccc3cc(-c4nc5cc(C(=O)N6C[C@H]7CC[C@@H]6[C@@H]7N)cc(F)c5n4C)n1c23] |
| 104 | 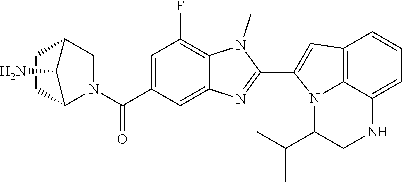 [SMILES: CC(C)C1CNc2cccc3cc(-c4nc5cc(C(=O)N6C[C@H]7CC[C@@H]6[C@@H]7N)cc(F)c5n4C)n1c23] |
| 105 | 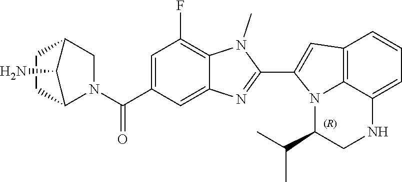 |
| 106 | 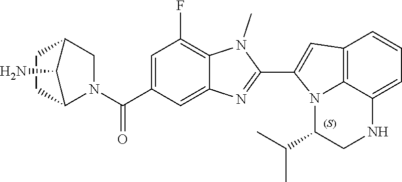 |
| 107 | 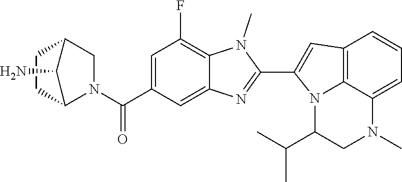 [SMILES: CC(C)C1CN(C)c2cccc3cc(-c4nc5cc(C(=O)N6C[C@H]7CC[C@@H]6[C@@H]7N)cc(F)c5n4C)n1c23] |
| 54 | 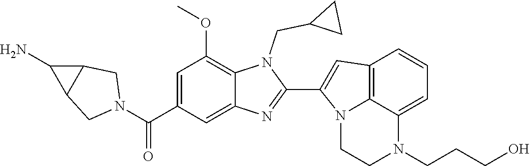 [SMILES: COc1cc(C(=O)N2CC3C(N)C3C2)cc2nc(-c3cc4cccc5c4n3CCN5CCCO)n(CC3CC3)c12] |
| 109 | 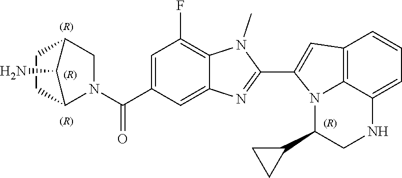 |
| 110 | 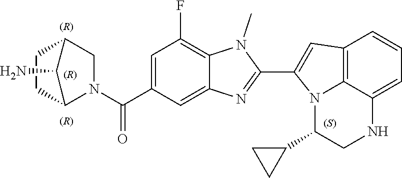 |
| 111 | 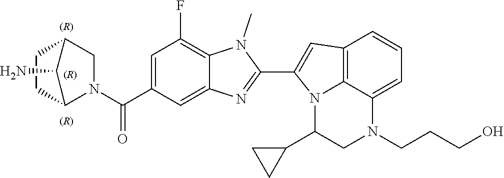 |
| 112 | 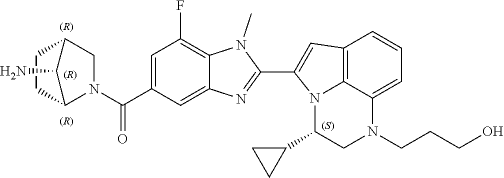 |
| 113 | 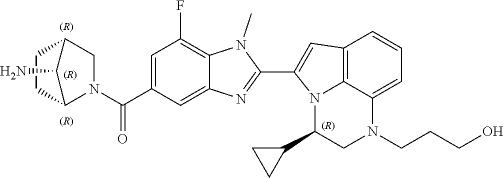 |
| 114 | 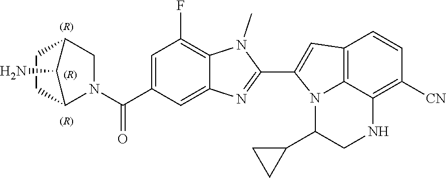 |
| 115 | 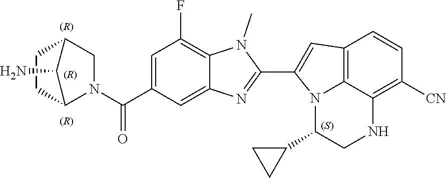 |
| 108 | 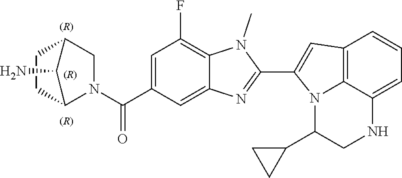 |
| 141 | 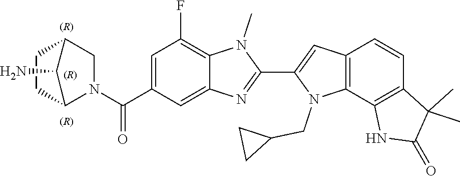 |
| 142 | 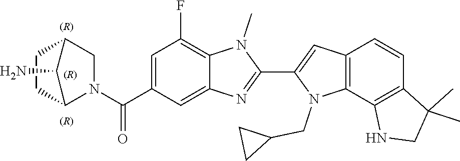 |
| 143 | 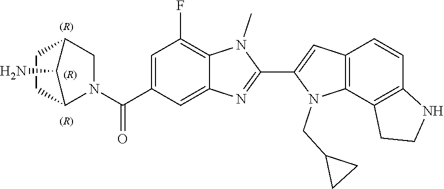 |
| 144 | 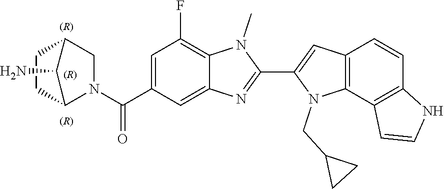 |
| 145 | 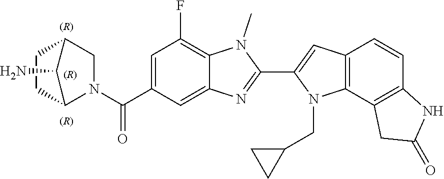 |
| 146 | 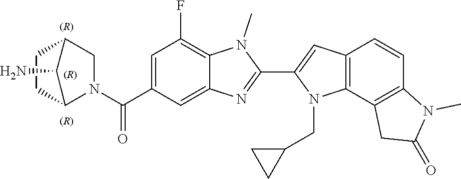 |
| 147 | 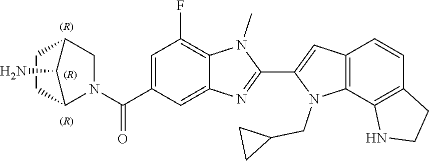 |
| 116 | 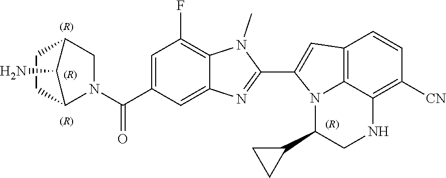 |
| 117 | 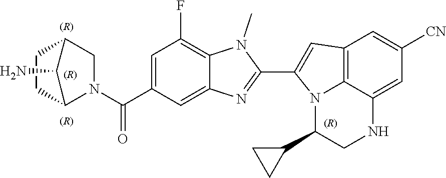 |
| 118 | 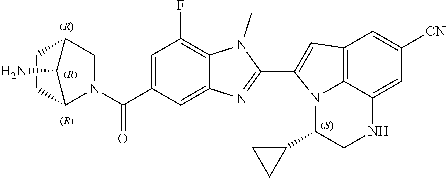 |
| 119 | 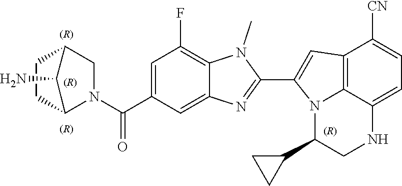 |
| 120 | 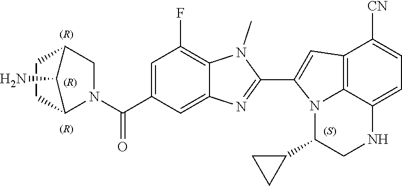 |
| 121 | 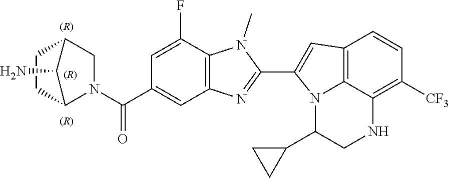 |
| 122 | 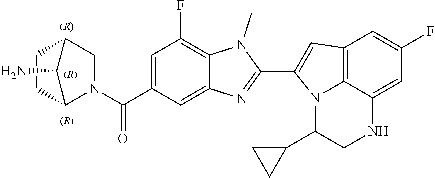 |
| 123 | 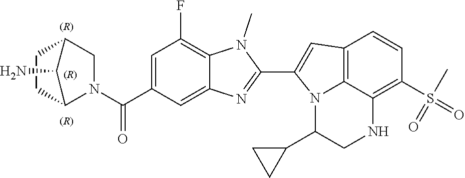 |
| 148 | 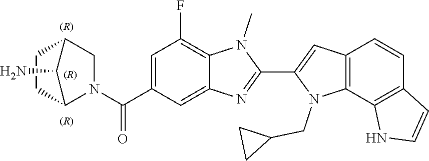 |
| 149 | 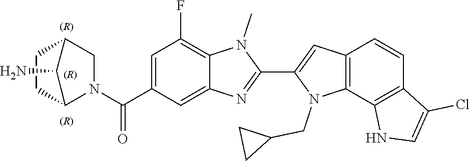 |
| 150 | 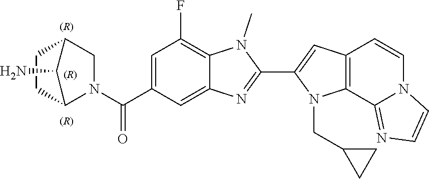 |
| 151 | 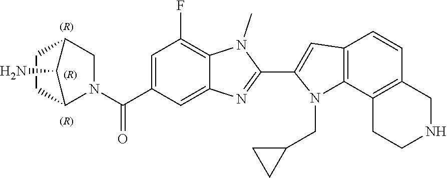 |
| 152 | 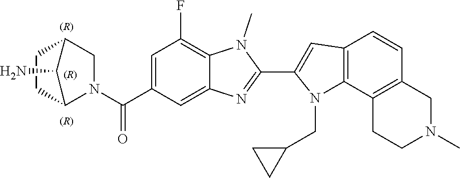 |
| 153 | 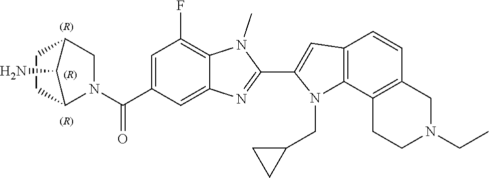 |
| 154 | 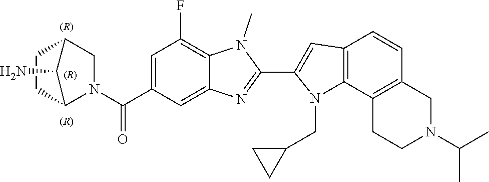 |
| 155 | 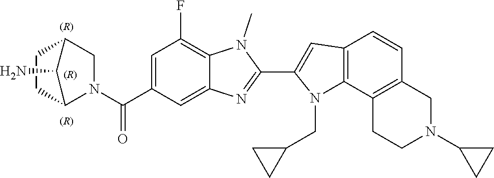 |
| 124 | 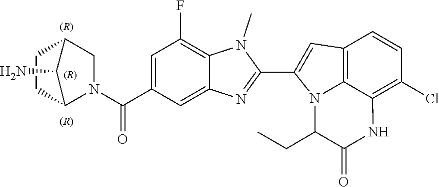 |
| 125 | 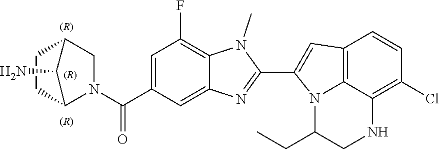 |
| 126 | 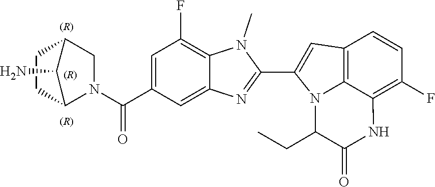 |
| 127 | 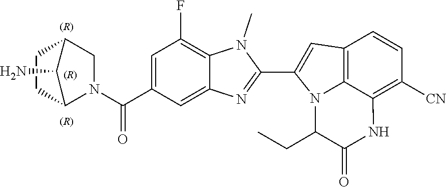 |
| 128 | 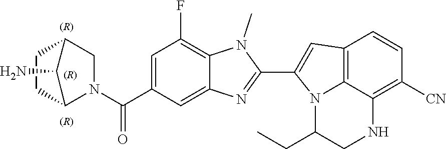 |
| 129 | 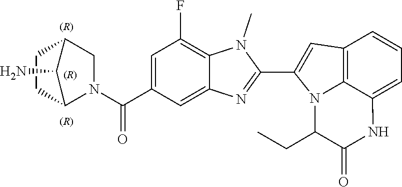 |
| 130 | 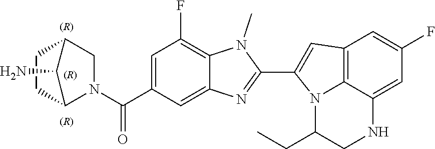 |
| 131 | 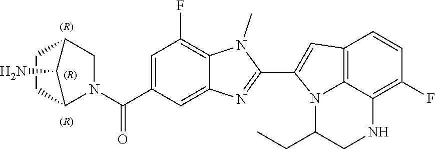 |
| 156 | 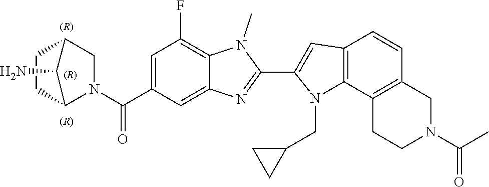 |
| 157 | 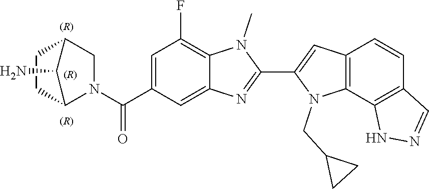 |
| 158 | 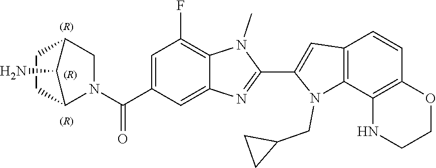 |
| 159 | 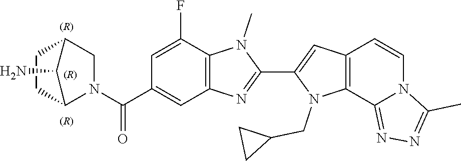 |
| 160 | 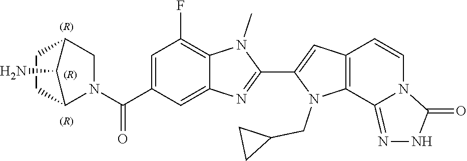 |
| 161 | 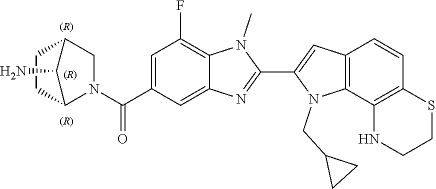 |
| 162 | 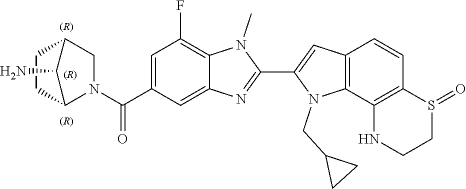 |
| 163 | 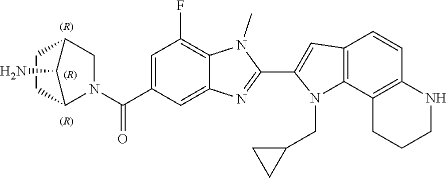 |
| 132 | 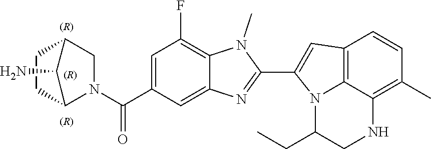 |
| 133 | 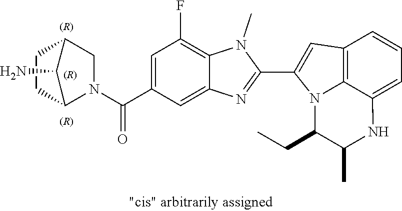 |
| 134 | 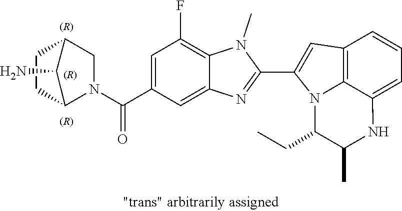 |
| 135 | 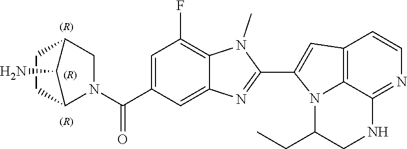 |
| 136 | 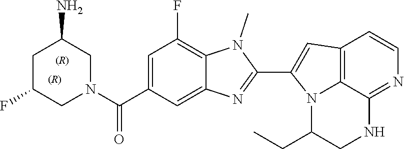 |
| 137 | 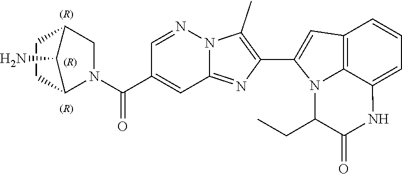 |
| 138 | 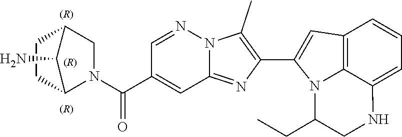 |
| 139 | 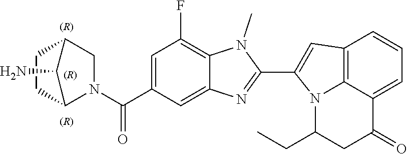 |
| 164 | 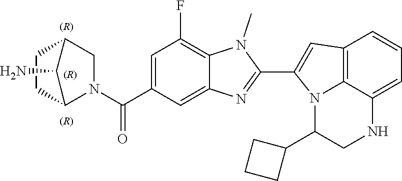 |
| 165 | 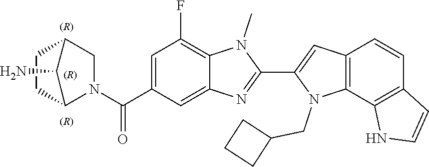 |
| 166 | 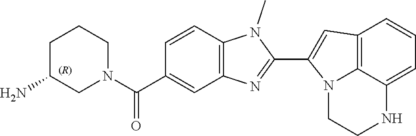 |
| 167 | 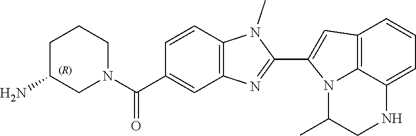 |
| 168 | 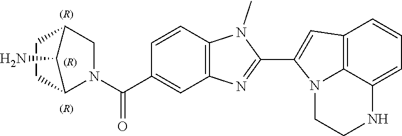 |
| 169 | 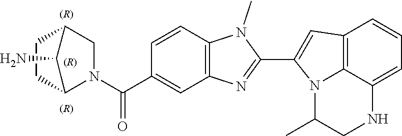 |
| 170 | 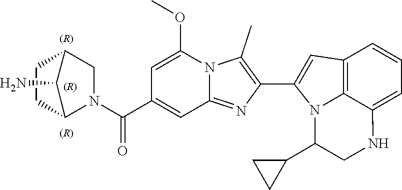 |
| 140 | 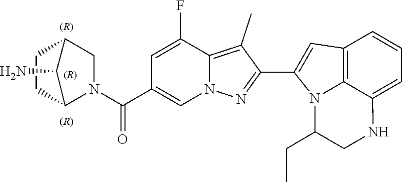 |

### 2. Definitions

The term “deuterium” or “D” refers to the isotopic abundance of D relative to H (hydrogen) is at least 50%, at least 75%, or at least 90%.

The term “halogen,” as used herein, refers to fluoride, chloride, bromide, or iodide.

The term “alkyl” used alone or as part of a larger moiety, such as “alkoxy” or “haloalkyl” and the like, means saturated aliphatic straight-chain or branched monovalent hydrocarbon radical of formula —CₙH₍₂ₙ₊₁₎. Unless otherwise specified, an alkyl group typically has 1-6 carbon atoms, i.e. C₁₋₆alkyl. As used herein, a “C₁₋₆alkyl” group means a radical having from 1 to 6 carbon atoms in a linear or branched arrangement. Examples include methyl, ethyl, n-propyl, iso-propyl, n-butyl, iso-butyl, tert-butyl, n-pentyl, isopentyl, hexyl, and the like.

The term “alkoxy” means an alkyl radical attached through an oxygen linking atom, represented by —O-alkyl. For example, “C₁₋₄alkoxy” includes methoxy, ethoxy, propoxy, and butoxy.

The term “haloalkyl” means alkyl, as the case may be, substituted with one or more halogen atoms. In one embodiment, the alkyl can be substituted by one to three halogens. Examples of haloalkyl, include, but are not limited to, trifluoromethyl, trichloromethyl, pentafluoroethyl and the like.

The term “hydroxylalkyl” means alkyl, as the case may be, substituted with one or more hydroxy groups.

The term “hydroxylalkoxyl” means alkoxyl, as the case may be, substituted with one or more hydroxy groups.

The term “methoxylalkyl” means alkyl, as the case may be, substituted with one or more methoxyl groups.

The term “methoxylalkoxyl” means alkoxyl, as the case may be, substituted with one or more methoxyl groups.

The term “alkylene” as used herein, means a straight or branched chain divalent hydrocarbon group of formula —CₙH₂ₙ—. Non-limiting examples include ethylene, and propylene.

The term “haloalkylene” means alkylene, as the case may be, substituted with one or more halogen atoms. In one embodiment, the alkylene can be substituted by one to three halogens.

The term “alkenyl” means an alkyl group in which one or more carbon/carbon single bond is replaced by a double bond.

The term “alkynyl” means an alkyl group in which one or more carbon/carbon single bond is replaced by a triple bond.

The term “alkyleneamine” means an alkyl group, as the case may be, substituted with one or more amine groups.

The term “alkyleneamide” means an alkyl group, as the case may be, substituted with one or more amide groups.

The term “alkylenecarbamate” means an alkyl group, as the case may be, substituted with one or more carbamate groups.

The term “alkyleneurea” means an alkyl group, as the case may be, substituted with one or more urea groups.

The term “benzoyl” means a phenylcarbonyl group.

The term “carbocyclyl” refers to any stable non-aromatic hydrocarbon ring having 3-12 membered carbocyclyl. In one embodiment, carbocyclyl is 3-, 4-, 5-, 6-, 7-, or 8-membered monocyclic or bicyclic or 7-, 8-, 9-, 10-, 11-, or 12-membered bicyclic or tricyclic hydrocarbon ring, any of which may be saturated, partially unsaturated, or unsaturated. Any substitutable ring atom can be substituted (e.g., by one or more substituents). Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, and cyclooctadienyl. In one embodiment, carbocyclyl is intended to include, bridged, fused, and spirocyclic rings. In a spirocyclic carbocyclyl, one atom is common to two different rings. An example of a spirocyclic carbocyclyl is spiro[3.3]heptanyl. In a bridged carbocyclyl, the rings share at least two common non-adjacent atoms. Examples of bridged carbocyclyls include bicyclo[2.2.1]heptanyl, bicyclo[2.2.1]hept-2-enyl, and adamantanyl. In a fused-ring carbocyclyl system, two or more rings may be fused together, such that two rings share one common bond. Examples of two- or three-fused ring carbocyclyls include naphthalenyl, tetrahydronaphthalenyl (tetralinyl), indenyl, indanyl (dihydroindenyl), anthracenyl, phenanthrenyl, and decalinyl. In one embodiment, carbocyclyl is 3-12 membered cycloalkyl (preferably 3-8 memebered cycloalkyl).

The term “cycloalkyl” refers to a cyclic, bicyclic, tricyclic, or polycyclic saturated hydrocarbon groups having 3 to 12 ring carbons. In one embodiment, cycloalkyl may have 3 to 7 ring cabons. Any substitutable ring atom can be substituted (e.g., by one or more substituents). Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl include: bicyclo[1.1.0]butane, bicyclo[2.1.0]pentane, bicyclo[1.1.0]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “heterocyclyl” or “heterocyclic” refers to a radical of a 3- to 12-membered non-aromatic ring system having ring carbon atoms and 1 to 4 ring heteroatoms, wherein each heteroatom is independently selected from nitrogen, quaternary nitrogen, oxidized nitrogen (e.g., NO), oxygen, and sulfur, including sulfoxide and sulfone (“3-12 membered heterocyclyl”). In some embodiments, a heterocyclyl group is a 3-7 membered non-aromatic ring system having ring carbon atoms and 1-4 ring heteroatoms, wherein each heteroatom is independently selected from nitrogen, oxygen, and sulfur (“3-7 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a bicyclic system (“bicyclic heterocyclyl”) or tricyclic system (“tricyclic heterocyclyl”); polycyclic ring systems include fused, bridged, or spiro ring systems). Exemplary monocyclic heterocyclyl groups include azetidinyl, oxetanyl, thietanyl, tetrahydrofuranyl, pyrrolidinyl, piperidinyl, tetrahydropyranyl, piperazinyl, morpholinyl, azepanyl, oxepanyl, thiepanyl, tetrahydropyridinyl, and the like. Heterocyclyl polycyclic ring systems can include heteroatoms in one or more rings in the polycyclic ring system. Substituents may be present on one or more rings in the polycyclic ring system.

Spiro heterocyclyl refers to 5 to 12 membered polycyclic heterocyclyl with rings connected through one common carbon atom (called as spiro atom), wherein said rings have one or more heteroatoms selected from the group consisting of nitrogen, quaternary nitrogen, oxidized nitrogen (e.g., NO), oxygen, and sulfur, including sulfoxide and sulfone, the remaining ring atoms being C, wherein one or more rings may contain one or more double bonds, but none of the rings has a completely conjugated pi-electron system. Representive examples of spiro heterocyclyl include, but are not limited to the following groups:

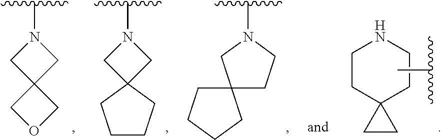

Fused heterocyclyl refers to a 5 to 12 membered polycyclic heterocyclyl group, wherein each ring in the group shares an adjacent pair of carbon atoms with another ring in the group, wherein one or more rings can contain one or more double bonds, but at least one of the rings is not an aromatic ring, and wherein said rings have one or more heteroatoms selected from the group consisting of nitrogen, quaternary nitrogen, oxidized nitrogen (e.g., NO), oxygen, and sulfur, including sulfoxide and sulfone, the remaining ring atoms being C. Representive examples of fused heterocyclyl include, but are not limited to the following groups:

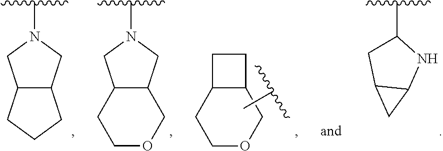

Bridged heterocyclyl refers to a 5 to 12 membered polycyclic heterocyclyl group, wherein any two rings in the group share two disconnected atoms, the rings can have one or more double bonds but have no completely conjugated π-electron system, and the rings have one or more heteroatoms selected from the group consisting of nitrogen, quaternary nitrogen, oxidized nitrogen (e.g., NO), oxygen, and sulfur, including sulfoxide and sulfone as ring atoms, the remaining ring atoms being C. Representive examples of bridged heterocyclyl include, but are not limited to the following groups:

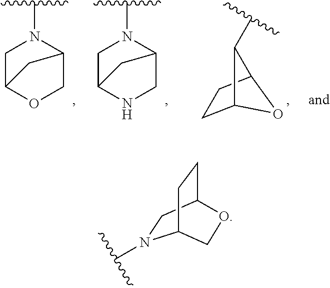

Generally, the carbocyclyl, the cycloalkyl, or the heterocyclyl may be unsubstituted, or be substituted with one or more substituents as valency allows, wherein the substituents can be independently selected from a number of groups such as oxo, —CN, halogen, alkyl and alkoxyl, opotionally, the alkyl substitution may be further substituted.

The term “aryl” refers to an all-carbon monocyclic ring or a polycyclic fused ring (a “fused” ring system means that each ring in the system shares an adjacent pair of carbon atoms with other ring in the system) group, and comprises a completely conjugated π-electron system. In one embodiment, the term “aryl” refers to a 6-12, 6-10, or 6 membered all-carbon monocyclic ring conprises a completely conjugated π-electron system. The term “aryl” may be used interchangeably with the terms “aryl ring” “carbocyclic aromatic ring”, “aryl group” and “carbocyclic aromatic group”. Representive examples of aryl are phenyl and naphthyl.

The term “heteroaryl,” as used herein, refers to a monocyclic or multicyclic aromatic hydrocarbon in which at least one of the ring carbon atoms has been replaced with a heteroatom independently selected from oxygen, nitrogen and sulfur. In one embodiment, the heteroaryl is based on a C₅₋₁₀ aryl with one or more of its ring carbon atoms replaced by the heteroatom. In one embodiment, heteroaryl refers to 5-12 membered, 5-10 membered, or 5-6 membered monocyclic aryl with one or more of its ring carbon atoms replaced by the heteroatom. A heteroaryl group may be attached through a ring carbon atom or, where valency permits, through a ring nitrogen atom. Generally, the heteroaryl may be unsubstituted, or be substituted with one or more substituents as valency allows with the substituents being independently selected from halogen, OH, alkyl, alkoxyl, and amino (e.g., NH₂, NHalkyl, N(alkyl)₂), optionally, the alkyl may be further substituted.

Examples of monocyclic 5-6 membered heteroaryl groups include furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), triazolyl (e.g., 2-triazolyl, 5-triazolyl), tetrazolyl (e.g., tetrazolyl), thienyl (e.g., 2-thienyl, 3-thienyl), pyrimidinyl, pyridinyl and pyridazinyl. Examples of polycyclic aromatic heteroaryl groups include carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, or benzisoxazolyl. A “substituted heteroaryl group” is substituted at any one or more substitutable ring atom, which is a ring carbon or ring nitrogen atom bonded to a hydrogen.

As used herein, many moieties (e.g., alkyl, alkylene, cycloalkyl, aryl, heteroaryl, or heterocyclyl) are referred to as being either “substituted” or “optionally substituted”. When a moiety is modified by one of these terms, unless otherwise noted, it denotes that any portion of the moiety that is known to one skilled in the art as being available for substitution can be substituted, which includes one or more substituents. Where if more than one substituent is present, then each substituent may be independently selected. Such means for substitution are well-known in the art and/or taught by the instant disclosure. The optional substituents can be any substituents that are suitable to attach to the moiety.

Where suitable substituents are not specifically enumerated, exemplary substituents include, but are not limited to: C₁₋₅alkyl, C₁₋₅hydroxyalkyl, C₁₋₅haloalkyl, C₁₋₅alkoxy, C₁₋₅ haloalkoxy, halogen, hydroxyl, cyano, amino, —CN, —NO₂, —OR^{c1}, —NR^{a1}R^{b1}, —S(O)ᵢR^{a1}, —NR^{a1}S(O)ᵢR^{b1}, —S(O)ᵢNR^{a1}R^{b1}, —C(═O)OR^{a1}, —OC(═O)OR^{a1}, —C(═S)OR^{a1}, —O(C═S)R^{a1}, —C(═O)NR^{a1}R^{b1}, —NR^{a1}C(═O)R^{b1}, —C(═S)NR^{a1}R^{b1}, —C(═O)R^{a1}, —C(═S)R^{a1}, NR^{a1}C(═S)R^{b1}, —O(C═O)NR^{a1}R^{b1}, —NR^{a1}(C═S)OR^{b1}, —O(C═S)NR^{a1}R^{b1}, —NR^{a1}(C═O)NR^{a1}R^{b1}, —NR^{a1}(C═S)NR^{a1}R^{b1}, phenyl, or 5-6 membered heteroaryl. Each R^{a1} and each R^{b1} are independently selected from —H and C₁₋₅alkyl, optionally substituted with hydroxyl or C₁₋₃alkoxy; R^{c1} is —H, C₁₋₅haloalkyl or C₁₋₅alkyl, wherein the C₁₋₅alkyl is optionally substituted with hydroxyl or C₁-C₃alkoxy.

The symbol

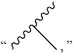

as used herein, refers to the point where the moiety attaches.

### Pharmaceutically Acceptable Salts

The term “pharmaceutically acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically acceptable salts are well known in the art. For example, S. M. Berge et al. describes pharmacologically acceptable salts in J. Pharm. Sci., 1977, 66, 1-19.

Pharmaceutically acceptable salts of the compounds of any one of the formulae described above include acid addition and base salts.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Pharmaceutically acceptable salts of compounds of any one of the formulae described above may be prepared by one or more of three methods:
(i) by reacting the compound of any one of the formulae described above with the desired acid or base;
(ii) by removing an acid- or base-labile protecting group from a suitable precursor of the compound of any one of the formulae described above or by ring-opening a suitable cyclic precursor, for example, a lactone or lactam, using the desired acid or base; or
(iii) by converting one salt of the compound of any one of the formulae described above to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of any one of the formulae described above, and pharmaceutically acceptable salts thereof, may exist in unsolvated and solvated forms.

### Stereoisomers and Other Variations

The compounds of any one of the formulae described above may exhibit one or more kinds of isomerism (e.g. optical, geometric or tautomeric isomerism). Such variation is implicit to the compounds of any one of the formulae described above defined as they are by reference to their structural features and therefore within the scope of the present disclosure.

Compounds having one or more chiral centers can exist in various stereoisomeric forms, i.e., each chiral center can have an R or S configuration, or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identifcal and are not mirror images of each other.

When a compound is designated by its chemical name (e.g., where the configuration is indicated in the chemical name by “R” or “S”) or its structure (e.g., the configuration is indicated by “wedge” bonds) that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

When two stereoisomers are depicted by their chemical names or structures, and the chemical names or structures are connected by an “and”, a mixture of the two stereoisomers is intended.

When two stereoisomers are depicted by their chemical names or structures, and the names or structures are connected by an “or”, one or the other of the two stereoisomers is intended, but not both.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

Racemic mixture means 50% of one enantiomer and 50% of the corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures of one or more diastereomers (e.g., racemic mixtures) of the compound.

The term “geometric isomer” means isomers that differ in the orientation of substituent atoms in relationship to a carbon-carbon double bond, to a carbocyclic ring, or to a bridged bicyclic system. Substituent atoms (other than hydrogen) on each side of a carbon-carbon double bond may be in an E or Z configuration according to the Cahn-Ingold-Prelog priority rules. In the “E” configuration, the substituents having the highest priorities are on opposite sides in relationship to the carbon-carbon double bond. In the “Z” configuration, the substituents having the highest priorities are oriented on the same side in relationship to the carbon-carbon double bond.

Substituents around a carbon-carbon double bond can also be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

Where structural isomers are interconvertible via a low energy barrier, tautomeric isomerism (“tautomerism”) can occur. This can take the form of proton tautomerism in compounds of any one of the formulae described above containing, for example, an imino, keto, or oxime group, or so-called valence tautomerism in compounds which contain an aromatic moiety. It follows that a single compound may exhibit more than one type of isomerism.

In certain instances, tautomeric forms of the disclosed compounds exist, such as the tautomeric structures shown below:

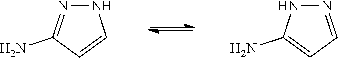 [SMILES: Nc1cc[nH]n1 ; Nc1ccn[nH]1]

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geomeric isomers in the mixture.

Cis/trans isomers may be separated by conventional techniques well known to those skilled in the art, for example, chromatography and fractional crystallisation.

Conventional techniques for the preparation/isolation of individual enantiomers/diastereomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound of any one of the formulae described above contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. Chiral compounds of any one of the formulae described above (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. Chiral chromatography using sub- and supercritical fluids may be employed. Methods for chiral chromatography useful in some embodiments of the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein). Columns can be obtained from Chiral Technologies, Inc, West Chester, Pa., USA, a subsidiary of Daicel® Chemical Industries, Ltd., Tokyo, Japan.

It must be emphasized that the compounds of any one of the formulae described above have been drawn herein in a single tautomeric form, all possible tautomeric forms are included within the scope of the present disclosure.

### 3. Administration and Dosing

Typically, a compound of the present disclosure is administered in an amount effective to treat a condition as described herein. The compounds of the present disclosure can be administered as compound per se, or alternatively, as a pharmaceutically acceptable salt. For administration and dosing purposes, the compound per se or pharmaceutically acceptable salt thereof will simply be referred to as the compounds of the present disclosure.

The compounds of the present disclosure are administered by any suitable route in the form of a pharmaceutical composition adapted to such a route, and in a dose effective for the treatment intended. The compounds of the present disclosure may be administered orally, rectally, vaginally, parenterally, or topically.

The compounds of the present disclosure may be administered orally. Oral administration may involve swallowing, so that the compound enters the gastrointestinal tract, or buccal or sublingual administration may be employed by which the compound enters the bloodstream directly from the mouth.

In another embodiment, the compounds of the present disclosure may also be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the present disclosure may also be administered topically to the skin or mucosa, that is, dermally or transdermally. In another embodiment, the compounds of the present disclosure can also be administered intranasally or by inhalation. In another embodiment, the compounds of the present disclosure may be administered rectally or vaginally. In another embodiment, the compounds of the present disclosure may also be administered directly to the eye or ear.

The dosage regimen for the compounds of the present disclosure and/or compositions containing said compounds is based on a variety of factors, including the type, age, weight, sex and medical condition of the patient; the severity of the condition; the route of administration; and the activity of the particular compound employed. Thus the dosage regimen may vary widely. In one embodiment, the total daily dose of a compound of the present disclosure is typically from about 0.001 to about 100 mg/kg (i.e., mg compound of the present disclosure per kg body weight) for the treatment of the indicated conditions discussed herein.

For oral administration, the compositions may be provided in the form of tablets containing 0.1-500 milligrams of the active ingredient for the symptomatic adjustment of the dosage to the patient. A medicament typically contains from about 0.01 mg to about 500 mg of the active ingredient. Intravenously, doses may range from about 0.01 to about 10 mg/kg/minute during a constant rate infusion.

Suitable subjects according to the present disclosure include mammalian subjects, including non-human mammal such as primates, rodents (mice, rats, hamsters, rabbits etc). In one embodiment, humans are suitable subjects. Human subjects may be of either gender and at any stage of development.

### 4. Pharmaceutical Compositions

In another embodiment, the present disclosure comprises pharmaceutical compositions. Such pharmaceutical compositions comprise a compound of the present disclosure presented, a pharmaceutically acceptable salt, or a stereoisomer thereof with a pharmaceutically acceptable carrier or excipient. Other pharmacologically active substances can also be present.

As used herein, “pharmaceutically acceptable carrier or excipient” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Pharmaceutically acceptable substances such as wetting agents or minor amounts of auxiliary substances such as wetting or emulsifying agents, preservatives or buffers, which enhance the shelf life or effectiveness of the antibody or antibody portion.

The compositions of present disclosure may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g. intravenous, subcutaneous, intraperitoneal, intramuscular). In another embodiment, the antibody is administered by intravenous infusion or injection. In yet another embodiment, the antibody is administered by intramuscular or subcutaneous injection.

Oral administration of a solid dose form may be, for example, presented in discrete units, such as hard or soft capsules, pills, cachets, lozenges, or tablets, each containing a predetermined amount of at least one compound of the present disclosure. In another embodiment, the oral administration may be in a powder or granule form. In another embodiment, the oral dose form is sublingual, such as, for example, a lozenge. In such solid dosage forms, the compounds of any one of the formulae described above are ordinarily combined with one or more adjuvants. Such capsules or tablets may contain a controlled release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present disclosure comprises a parenteral dose form.

“Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

In another embodiment, the present disclosure comprises a topical dose form.

“Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of present disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Finnin and Morgan, J. Pharm. Sci., 88: 955-958, 1999.

Formulations suitable for topical administration to the eye include, for example, eye drops wherein the compound of present disclosure is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the compounds of the present disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present disclosure comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the present disclosure may be prepared by any of the well-known techniques of pharmacy, such as effective formulation and administration procedures.

The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

### 5. Method of Treatment

Compounds and compositions described herein are generally useful for the inhibition of PAD4.

The activity of a compound utilized in present disclosure as an inhibitor of PAD4, may be assayed in vitro, in vivo or in a cell line. In vitro assays include assays that determine the inhibition of PAD4. Detailed conditions for assaying a compound utilized in this present disclosure as an inhibitor of PAD4 are set forth in the Examples below. In some embodiments, a provided compound inhibits PAD4 selectively as compared to PAD2.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of PAD4 and are therefore useful for treating one or more diseases or disorders associated with PAD4 enzyme activity. Thus, in certain embodiments, the present disclosure provides a method for treating a disease or a disorder associated/mediated with PAD4 enzyme activity, comprising the step of administering to a patient in need thereof a compound of the present disclosure, or a pharmaceutically acceptable composition thereof.

In one embodiment, a disease or a disorder associated/mediated with PAD4 enzyme activity is a disease, condition, or disorder mediated by inappropriate PAD4 activity. In some embodiments, a disease or a disorder associated/mediated with PAD4 enzyme activity is selected from the group consisting of rheumatoid arthritis, vasculitis, systemic lupus erythematosus, ulcerative colitis, cancer, cystic fibrosis, asthma, cutaneous lupus erythematosus, and psoriasis. In a further embodiment, the disease or a disorder associated with PAD4 enzyme activity is rheumatoid arthritis. In a further embodiment, the disease or a disorder associated with PAD4 enzyme activity is systemic lupus. In a further embodiment, the disease or a disorder associated with PAD4 enzyme activity is vasculitis. In a further embodiment, the disease or a disorder associated with PAD4 enzyme activity cutaneous lupus erythematosus. In a further embodiment, the disease or a disorder associated with PAD4 enzyme activity is psoriasis.

In one embodiment, the present disclosure provides a method for treating a subject with a disease or condition comprising administering to the subject a therapeutically effective amount of a compound of the present disclosure, or a pharmaceutically acceptable salt or a stereoisomer thereof, wherein said disease or condition is a bacterial infection, a viral infection, a metabolic disease, an autoimmune disease, an autoinflammatory disease, cancer, or a septic condition.

In one embodiment, the present disclosure provides a method for treating a subject with a disease or condition comprising administering to the subject a therapeutically effective amount of a compound of the present disclosure, or a pharmaceutically acceptable salt or a stereoisomer thereof, wherein said disease or condition is a lung infectious disease (e.g. Covid-19), acute lymphocytic leukemia, ankylosing spondylitis, asthma, breast cancer, lung cancer, colorectal cancer, pancreatic cancer, blood cancer, neurological cancer, cutaneous cancers, chronic lymphocytic leukemia, cutaneous lupus erythematosis, gout, inflammatory bowel disease (IBD), type 2 diabetes, obesity, type 1 diabetes mellitus (T1DM), cystic fibrosis, multiple sclerosis, psoriasis, rheumatoid arthritis, systemic lupus erythematosus, ulcerative colitis, or vasculitis.

In one embodiment, the present disclosure provides a method for treating a subject with a disease or condition comprising administering to the subject a therapeutically effective amount of a compound of the present disclosure, or a pharmaceutically acceptable salt or a stereoisomer thereof, wherein said disease or condition is cancer and the cancer is metastasized.

In one embodiment, the present disclosure provides a method of treatment of rheumatoid arthritis, vasculitis, systemic lupus erythematosus, ulcerative colitis, cystic fibrosis, asthma, gout, cutaneous lupus erythematosus, or psoriasis, which method comprises administering to a human subject in need thereof, a therapeutically effective amount of a provided compound, a pharmaceutically acceptable salt thereof, or a stereoisomer thereof.

In one embodiment there is provided a method of treatment of rheumatoid arthritis, which method comprises administering to a human subject in need thereof, a therapeutically effective amount of a provided compound, a pharmaceutically acceptable salt thereof, or a stereoisomer thereof.

In one embodiment there is provided a method of treatment of systemic lupus erythematosus, which method comprises administering to a human subject in need thereof, a therapeutically effective amount of a provided compound, a pharmaceutically acceptable salt thereof, or a stereoisomer thereof.

In one embodiment there is provided a method of treatment of vasculitis, which method comprises administering to a human subject in need thereof, a therapeutically effective amount of a provided compound, a pharmaceutically acceptable salt thereof, or a stereoisomer thereof. In one embodiment there is provided a method of treatment of cutaneous lupus erythematosus, which method comprises administering to a human subject in need thereof, a therapeutically effective amount of a provided compound, a pharmaceutically acceptable salt thereof, or a stereoisomer thereof. In one embodiment there is provided a method of treatment of psoriasis, which method comprises administering to a human subject in need thereof, a therapeutically effective amount of a provided compound, a pharmaceutically acceptable salt thereof, or a stereoisomer thereof.

In some embodiments, a disease or a disorder associated with PAD4 enzyme activity is selected from the group consisting of acid-induced lung injury, acne (PAPA), acute lymphocytic leukemia, acute respiratory distress syndrome, Addison's disease, adrenal hyperplasia, adrenocortical insufficiency, ageing, AIDS, alcoholic hepatitis, alcoholic liver disease, allergen induced asthma, allergic bronchopulmonary, aspergillosis, allergic conjunctivitis, alopecia, Alzheimer's disease, amyloidosis, amyotropic lateral sclerosis, weight loss, angina pectoris, angioedema, anhidrotic ecodermal dysplasia-ID, ankylosing spondylitis, anterior segment, inflammation, antiphospholipid syndrome, aphthous stomatitis, appendicitis, arthritis, asthma, atherosclerosis, atopic dermatitis, autoimmune diseases, autoimmune hepatitis, bee sting-induced inflammation, Bechet's disease, Bechet's syndrome, Bells Palsey, berylliosis, Blau syndrome, bone pain, bronchiolitis, burns, bursitis, cancer, cardiac hypertrophy, carpal tunnel syndrome, catabolic disorders, cataracts, cerebral aneurysm, chemical irritant-induced inflammation, chorioretinitis, chronic heart failure, chronic lung disease of prematurity, chronic lymphocytic leukemia, chronic obstructive pulmonary disease, colitis, complex regional pain syndrome, connective tissue disease, corneal ulcer, crohn's disease, cryopyrin-associated periodic syndromes, cyrptococcosis, cystic fibrosis, deficiency of the interleukin-1-receptor antagonist (DIRA), dermatitis, dermatitis endotoxemia, dermatomyositis, diffuse intrinsic pontine glioma, endometriosis, endotoxemia, epicondylitis, erythroblastopenia, familial amyloidotic polyneuropathy, familial cold urticarial, familial Mediterranean fever, fetal growth retardation, glaucoma, glomerular disease, glomerular nephritis, gout, gouty arthritis, graft-versus-host disease, gut diseases, head injury, headache, hearing loss, heart disease, hemolytic anemia, Henoch-Scholein purpura, hepatitis, hereditary periodic fever syndrome, herpes zoster and simplex, HIV-1, Hodgkin's disease, Huntington's disease, hyaline membrane disease, hyperammonemia, hypercalcemia, hypercholesterolemia, hyperimmunoglobulinemia D with recurrent fever (HIDS), hypoplastic and other anemias, hypoplastic anemia, idiopathic thrombocytopenic purpura, incontinentia pigmenti, infectious mononucleosis, inflammatory bowel disease, inflammatory lung disease, inflammatory neuropathy, inflammatory pain, insect bite-induced inflammation, iritis, irritant-induced inflammation, ischemia/reperfusion, juvenile rheumatoid arthritis, keratitis, kidney disease, kidney injury caused by parasitic infections, kidney injury caused by parasitic infections, kidney transplant rejection prophylaxis, leptospiriosis, leukemia, Loeffler's syndrome, lung injury, lupus, lupus nephritis, lymphoma, meningitis, mesothelioma, mixed connective tissue disease, Muckle-Wells syndrome (urticaria deafness amyloidosis), multiple sclerosis, muscle wasting, muscular dystrophy, myasthenia gravis, myocarditis, mycosis fungoides, myelodysplastic syndrome, myositis, nasal sinusitis, necrotizing enterocolitis, neonatal onset multisystem inflammatory disease (NOMID), nephrotic syndrome, neuritis, neuropathological diseases, non-allergen induced asthma, obesity, ocular allergy, optic neuritis, organ transplant, osteoarthritis, otitis media, Paget's disease, pain, pancreatitis, Parkinson's disease, pemphigus, pericarditis, periodic fever, periodontitis, peritoneal endometriosis, pertussis, pharyngitis and adenitis (PFAPA syndrome), plant irritant-induced inflammation, pneumonia, pneumonitis, pneumosysts infection, poison ivy/urushiol oil-induced inflammation, polyarteritis nodosa, polychondritis, polycystic kidney disease, polymyositis, psoriasis, psychosocial stress diseases, pulmonary disease, pulmonary hypertension, pulmonary fibrosis, pyoderma gangrenosum, pyogenic sterile arthritis, renal disease, retinal disease, rheumatic carditis, rheumatic disease, rheumatoid arthritis, sarcoidosis, seborrhea, sepsis, severe pain, sickle cell, sickle cell anemia, silica-induced disease, Sjogren's syndrome, skin diseases, sleep apnea, solid tumors, spinal cord injury, Stevens-Johnson syndrome, stroke, subarachnoid hemorrhage, sunburn, temporal arteritis, tenosynovitis, thrombocytopenia, thyroiditis, tissue transplant, TNF receptor associated periodic syndrome (TRAPS), toxoplasmosis, transplant, traumatic brain injury, tuberculosis, type 1 diabetes, type 2 diabetes, ulcerative colitis, urticarial, uveitis, Wegener's granulomatosis, interstitial lung disease, psoriatic arthritis, juvenile idiopathic arthritis, Sjögren's syndrome, antineutrophil cytoplasmic antibody (ANCA)-associated vasculitis, antiphospholipid antibody syndrome, sepsis, deep vein thrombosis, fibrosis, Alzheimer's, scleroderma and CREST syndrome.

In one embodiment, the present disclosure provides a compound, a pharmaceutically acceptable salt thereof, or a stereoisomer thereof, for use in therapy. In another embodiment, the present disclosure provides a compound, a pharmaceutically acceptable salt thereof, or a stereoisomer thereof, for use in the treatment of a disease or a disorder mediated by inappropriate PAD4 activity. In another embodiment, the present disclosure provides a compound, a pharmaceutically acceptable salt thereof, or a stereoisomer thereof, for use in the treatment of rheumatoid arthritis, vasculitis, systemic lupus erythematosus, ulcerative colitis, cancer, cystic fibrosis, asthma, cutaneous lupus erythematosus, or psoriasis. In another embodiment, the present disclosure provides a compound, a pharmaceutically acceptable salt thereof, or a stereoisomer thereof, for use in the treatment of rheumatoid arthritis. In another embodiment, the present disclosure provides a compound, a pharmaceutically acceptable salt thereof, or a stereoisomer thereof, for use in the treatment of systemic lupus. In another embodiment, the present disclosure provides a compound, a pharmaceutically acceptable salt thereof, or a stereoisomer thereof, for use in the treatment of vasculitis. In another embodiment, the present disclosure provides a compound, a pharmaceutically acceptable salt thereof, or a stereoisomer thereof, for use in the treatment of cutaneous lupus erythematosus. In another embodiment, the present disclosure provides a compound, a pharmaceutically acceptable salt thereof, or a stereoisomer thereof, for use in the treatment of psoriasis. In another embodiment, the present disclosure provides the use of a compound, a pharmaceutically acceptable salt thereof, or a stereoisomer thereof, in the manufacture of a medicament for use in the treatment of a disorder mediated by inappropriate PAD4 activity. In another embodiment, the present disclosure provides the use of a compound of formula (I0), (I), (II), (III), (IIIA), (IV), or (V), a pharmaceutically acceptable salt thereof, or a stereoisomer thereof, in the manufacture of a medicament for use in the treatment of rheumatoid arthritis, vasculitis, systemic lupus erythematosus, ulcerative colitis, cancer, cystic fibrosis, asthma, cutaneous lupus erythematosus, or psoriasis. In another embodiment, the present disclosure provides the use of a compound of formula (I0), (I), (II), (III), (IIIA), (IV), or (V), a pharmaceutically acceptable salt thereof, or a stereoisomer thereof, in the manufacture of a medicament for use in the treatment of rheumatoid arthritis. In another embodiment, the present disclosure provides the use of a compound of formula (I0), (I), (II), (III), (IIIA), (IV), or (V), a pharmaceutically acceptable salt thereof, or a stereoisomer thereof, in the manufacture of a medicament for use in the treatment of systemic lupus. In another embodiment, the present disclosure provides the use of a compound of formula (I0), (I), (II), (III), (IIIA), (IV), or (V), a pharmaceutically acceptable salt thereof, or a stereoisomer thereof, in the manufacture of a medicament for use in the treatment of vasculitis. In another embodiment, the present disclosure provides the use of a compound of formula (I0), (I), (II), (III), (IIIA), (IV), or (V), a pharmaceutically acceptable salt thereof, or a stereoisomer thereof, in the manufacture of a medicament for use in the treatment of cutaneous lupus erythematosus. In another embodiment, the present disclosure provides the use of a compound of formula (I0), (I), (II), (III), (IIIA), (IV), or (V), a pharmaceutically acceptable salt thereof, or a stereoisomer thereof, in the manufacture of a medicament for use in the treatment of psoriasis. In a further embodiment, the present disclosure provides a pharmaceutical composition for the treatment or prophylaxis of a disease or a disorder mediated by inappropriate PAD4 activity comprising a provided compound, a pharmaceutically acceptable salt thereof, or a stereoisomer thereof. In a further embodiment, the present disclosure provides a pharmaceutical composition for the treatment or prophylaxis of rheumatoid arthritis, vasculitis, systemic lupus erythematosus, ulcerative colitis, cancer, cystic fibrosis, asthma, cutaneous lupus erythematosus, or psoriasis, comprising a provided compound, a pharmaceutically acceptable salt thereof, or a stereoisomer thereof. In a further embodiment, the present disclosure provides a pharmaceutical composition for the treatment or prophylaxis of rheumatoid arthritis comprising a provided compound, a pharmaceutically acceptable salt thereof, or a stereoisomer thereof. In a further embodiment, the present disclosure provides a pharmaceutical composition for the treatment or prophylaxis of systemic lupus comprising a provided compound, a pharmaceutically acceptable salt thereof, or a stereoisomer thereof. In a further embodiment, the present disclosure provides a pharmaceutical composition for the treatment or prophylaxis of vasculitis comprising a provided compound, a pharmaceutically acceptable salt thereof, or a stereoisomer thereof. In a further embodiment, the present disclosure provides a pharmaceutical composition for the treatment or prophylaxis of cutaneous lupus erythematosus comprising a provided compound, a pharmaceutically acceptable salt thereof, or a stereoisomer thereof. In a further embodiment, the present disclosure provides a pharmaceutical composition for the treatment or prophylaxis of psoriasis comprising a provided compound, a pharmaceutically acceptable salt thereof, or a stereoisomer thereof.

### 6. Treatment Kits

One aspect of the present disclosure relates to a kit for conveniently and effectively carrying out the methods or uses in accordance with the present disclosure. In general, the pharmaceutical pack or kit comprises one or more containers filled with one or more of the ingredients of the pharmaceutical compositions of the present disclosure. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Optionally associated with such container(s) can be a notice in the form prescribed by a governmental agency regulating the manufacture, use or sale of pharmaceutical products, which notice reflects approval by the agency of manufacture, use or sale for human administration.

The following representative examples contain important additional information, exemplification and guidance which can be adapted to the practice of this disclosure in its various embodiments and the equivalents thereof. These examples are intended to help illustrate the present disclosure, and are not intended to, nor should they be construed to, limit its scope. Indeed, various modifications of the present disclosure, and many further embodiments thereof, in addition to those shown and described herein, will become apparent to those skilled in the art upon review of this document, including the examples which follow and the references to the scientific and patent literature cited herein.

The contents of the cited references are incorporated herein by reference to help illustrate the state of the art.

In addition, for purposes of this disclosure, the chemical elements are identified in accordance with the Periodic Table of the Elements, CAS version, Handbook of Chemistry and Physics, 75^{th} Ed., inside cover. Additionally, general principles of organic chemistry, as well as specific functional moieties and reactivity, are described in “Organic Chemistry,” Thomas Sorrell, University Science Books, Sausalito: 1999, and “Organic Chemistry,” Morrison & Boyd (3d Ed), the entire contents of both of which are incorporated herein by reference.

### 7. Preparation

The compounds of any one of the formulae described above, may be prepared by the general and specific methods described below, using the common general knowledge of one skilled in the art of synthetic organic chemistry. Such common general knowledge can be found in standard reference books such as Comprehensive Organic Chemistry, Ed. Barton and Ollis, Elsevier; Comprehensive Organic Transformations: A Guide to Functional Group Preparations, Larock, John Wiley and Sons; and Compendium of Organic Synthetic Methods, Vol. I-XII (published by Wiley-Interscience). The starting materials used herein are commercially available or may be prepared by routine methods known in the art.

In the preparation of the compounds of any one of the formulae described above, it is noted that some of the preparation methods described herein may require protection of remote functionality (e.g., primary amine, secondary amine, carboxyl in any one of the formulae described above precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines, and lower alkyl or benzyl esters for carboxylic acids) which are generally not chemically reactive under the reaction conditions described and can typically be removed without chemically altering other functionality in the any one of the formulae described above compounds.

The Schemes described below are intended to provide a general description of the methodology employed in the preparation of the compounds of the present disclosure. Some of the compounds of the present present disclosure may contain single or multiple chiral centers with the stereochemical designation (R) or (S). It will be apparent to one skilled in the art that all of the synthetic transformations can be conducted in a similar manner whether the materials are enantioenriched or racemic. Moreover, the resolution to the desired optically active material may take place at any desired point in the sequence using well known methods such as described herein and in the chemistry literature.

### EXAMPLES

### Abbreviations

AcOH Acetic Acid
BH₃ Borane
Boc₂O Di-tert-butyl dicarbonate
BrettPhos-Pd-G3 Methanesulfonato2-Dicyclohexylphosphino-3,6-dimethoxy-2′-4′-6′-tri-i-propyl-1,1′-bipheny)(2′-amino-1,1′-biphenyl-2-yl)palladium(II)
CH₃I Iodomethane
CHCl₃ Chloroform
Cs₂CO₃ Cesium Carbonate
CuCN Copper(I) Cyanide
DCE 1,2-Dichloroethane
DCM Dichloromethane
DDQ 1,2-Dichloro-4,5-Dicyanobenzoquinone
DIPEA, DIEA N,N-Diisopropylethylamine
DMF Dimethylformamide
DMSO Dimethyl Sulfoxide
DPPF 1,1′-Bis(diphenylphosphino)ferrocene
EA, EtOAc Ethyl acetate
H Hour
H₂ Hydrogen
HATU Hexafluorophosphate Azabenzotriazole Tetramethyl Uronium
HCl Hydrochloric Acid
H₃PO₄ Phosphoric Acid
HOAC Acetic Acid
IPA Isopropyl Alcohol
K₂CO₃ Potassium Carbonate
K₂S₂O₄ Potassium Dithionite
LCMS Liquid Chromatograph Mass Spectrometer
LiAlH₄ Lithium Aluminium Hydride
LiOH Lithium Hydroxide
MeCN Methyl Cyanide
MeOH Methanol
MgSO4 Magnesium Sulfate
Min Minute
MnO₂ Manganese Dioxide
NaH Sodium hydride
Na₂CO₃ Sodium Carbonate
NaNO₂ Sodium nitrite
NaOH Sodium Hydroxide
NH₄Cl Ammonium chloride
NaOtBu Sodium tert-butoxide
Na₂SO₄ Sodium Sulfate
Na₂S₂O₄ Sodium Dithionite
NMP N-Methyl-2-pyrrolidone
Pd—C Palladium on Carbon
Pd₂(dba)₃ Tris(dibenzylideneacetone)dipalladium(0)
PE Petroleum Ether
Prep-HPLC Preparative-Scale High Performance Liquid Chromatography
Prep-TLC Preparative Thin Layer Chromatography
POCl₃ Phosphorus Oxychloride
RT Room Temperature
SFC Supercritical Fluid Chromatography
TBME tert-Butyl Methyl Ether
TEA Triethanolamine
TFA Trifluoroacetic Acid
THF Tetrahydrofuran
tBuXPhos-Pd-G3 [2′-(Amino)[1,1′-biphenyl]-2-yl][bis(1,1-dimethylethyl)[2′,4′,6′-tris(1-methylethyl)[1,1′-biphenyl]-2-yl]phosphine](methanesulfonatChemicalbooko)palladium; [(2-Di-tert-butylphosphino-2′,4′,6′-triisopropyl-1,1′-biphenyl)-2-(2′-amino-1,1′-biphenyl)]palladium(II) methanesulfonate
pTSA p-Toluenesulfonic Acid
Zn(CN)₂ Zinc Cyanide

### Synethic Examples

### Example 1 Synthesis of N-(3-(5-(5-((3R,5R)-3-amino-5-fluoropiperidine-1-carbonyl)-7-methoxy-1-methyl-1H-benzo[d]imidazol-82-yl)-2,3-dihydro-1H-pyrrolo[1,2,3-de]quinoxalin-1-yl)propyl)acetamide

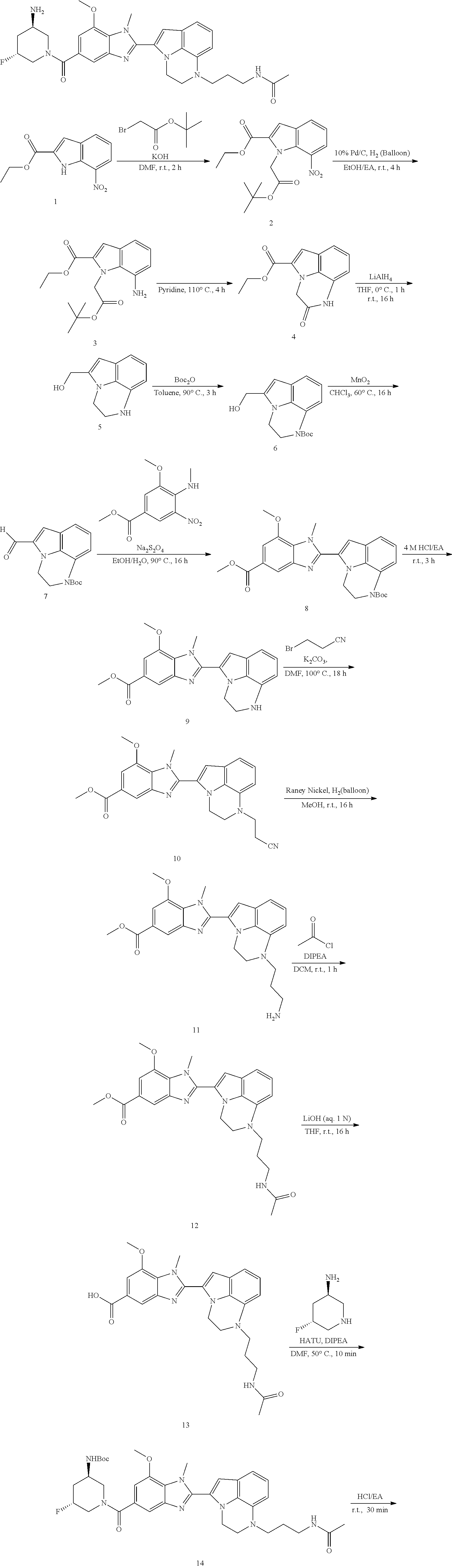

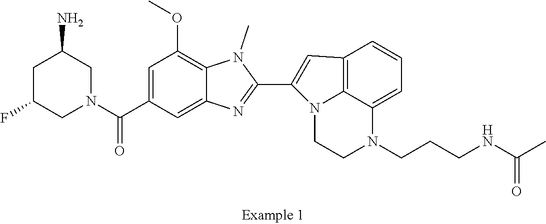 [SMILES: COc1cc(C(=O)N2C[C@H](N)C[C@@H](F)C2)cc2nc(-c3cc4cccc5c4n3CCN5CCCNC(C)=O)n(C)c12]

### Step 1:

To a solution of ethyl 7-nitro-1H-indole-2-carboxylate (6.0 g, 25.62 mmol) in anhydrous DMF (50 mL) were added KOH (1.87 g, 33.30 mmol, 916.02 μL), and tert-butyl 2-bromoacetate (5.75 g, 29.46 mmol, 4.32 mL). The reaction mixture was stirred at RT for 2 h, diluted with EA (200 mL), washed with water (20 mL*3) and brine (20 mL). The organic phase was dried over anhydrous sodium sulfate and filtered. The filtrate was concentrated in vacuo, and the residue was purified by flash column chromatography on silica gel (eluting with EA/PE from 0 to 20%) to provide ethyl 1-(2-tert-butoxy-2-oxo-ethyl)-7-nitro-indole-2-carboxylate (8.2 g, 23.54 mmol, 91.89% yield) as a yellow solid. LC/MS(ESI⁺) [(M-55)⁺]: 292.8.

### Step 2:

A mixture of ethyl 1-(2-tert-butoxy-2-oxo-ethyl)-7-nitro-indole-2-carboxylate (1.1 g, 3.16 mmol) and Palladium 10% on Carbon (200 mg, 631.55 μmol) was dissolved in THF (20 mL) under the atmosphere of Hydrogen (Balloon) (3.16 mmol) at RT for 4 h until the starting material was consumed and desired signal was found by LC/MS. The reaction mixture was filtered through Celite, and the filtrate was evaporated under reduced pressure in vacuo. The residue was purified by flash column chromatography on silica gel (eluting with EA/PE from 0 to 80%) to afford ethyl 7-amino-1-(2-tert-butoxy-2-oxo-ethyl) indole-2-carboxylate (850 mg, 2.67 mmol, 84.55% yield) as a white solid. LC/MS(ESI⁺) [(M-55)⁺]: 262.8.

### Step 3:

Ethyl 7-amino-1-(2-tert-butoxy-2-oxo-ethyl)indole-2-carboxylate (850 mg, 2.67 mmol) was dissolved in pyridine (20 mL). The reaction mixture was stirred and refluxed at 110° C. for 4 h. After removal of the solvent in vacuo, the residue was purified by flash column chromatography on silica gel (eluting with EA/PE from 0 to 50%) to provide ethyl 10-oxo-1,9-diazatricyclo[6.3.1.04,12]dodeca-2,4,6,8(12)-tetraene-2-carboxylate (500 mg, 2.05 mmol, 76.67% yield) as a light yellow solid. LC/MS(ESI⁺) [(M+H)⁺]: 244.8.

### Step 4:

To a solution of ethyl 10-oxo-1,9-diazatricyclo[6.3.1.04,12]dodeca-2,4,6,8(12)-tetraene-2-carboxylate (4.5 g, 18.42 mmol) in THF (50 mL) was added lithium aluminum hydride at 0° C. (2.10 g, 55.35 mmol). After 1 h at 0° C., the mixture was stirred overnight at rt. The mixture was then slowly mixed with 1 mL of water, 1 mL of sodium hydroxide solution (15%) and 3 mL of water. The resulting suspension was then decanted from a solid and the supernatant was collected. The solid was treated briefly with tetrahydrofuran (50 mL) and ethyl acetate (50 mL) in an ultrasonic bath and then decanted. The combined supernatants were mixed with water and the phases were separated. The organic phase was washed once with saturated sodium chloride solution and then dried with sodium sulfate. After filtration the reaction mixture was concentrated under vacuum to give 1,9-diazatricyclo [6.3.1.04,12]dodeca-2,4,6,8(12)-tetraen-2-ylmethanol (2.7 g, 14.34 mmol, 77.86% yield) as a light yellow oil, which was used without further purification in the next step. LC/MS (ESI⁺) [(M+H)⁺]: 188.9.

### Step 5:

A mixture of 1,9-diazatricyclo [6.3.1.04,12]dodeca-2,4,6,8(12)-tetraen-2-ylmethanol (2.7 g, 14.34 mmol) and Di-tert-butyl decarbonate (6.25 g, 28.62 mmol, 6.57 mL) was dissolved in toluene (50 mL). The resulting mixture was stirred at 90° C. for 3 h. Desired signal was found by LC/MS. After removal of the solvent and the residue was purified by flash column chromatography on silica gel (eluting with EA/PE from 0 to 40%) to provide tert-butyl 2-(hydroxymethyl)-1,9-diazatricyclo [6.3.1.04,12]dodeca-2,4,6,8(12)-tetraene-9-carboxylate (3.5 g, 12.14 mmol, 84.62% yield) as colorless liquid oil. LC/MS(ESI⁺) [(M+H)⁺]: 288.9.

### Step 6:

To tert-butyl 2-(hydroxymethyl)-1,9-diazatricyclo [6.3.1.04,12]dodeca-2,4,6,8(12)-tetraene-9-carboxylate (2.7 g, 9.36 mmol) in chloroform (30 mL) was added Manganese dioxide (2.44 g, 28.09 mmol). The suspension was stirred at rt overnight then filtered through celite washing with 30 mL of chloroform. The filtrate was concentrated in vacuo to afford the crude product. The crude product was purified by flash column chromatography on silica gel (eluting with EA/PE from 0 to 50%) to give tert-butyl 2-formyl-1,9-diazatricyclo [6.3.1.04,12]dodeca-2,4,6,8(12)-tetraene-9-carboxylate (2.5 g, 8.73 mmol) as a colorless oil. LC/MS(ESI⁺) [(M+H)⁺]: 286.8.

### Step 7:

A mixture of tert-butyl 2-formyl-1,9-diazatricyclo [6.3.1.04,12]dodeca-2,4,6,8(12)-tetraene-9-carboxylate (2.5 g, 8.73 mmol), methyl 3-methoxy-4-(methylamino)-5-nitro-benzoate (2.1 g, 8.73 mmol) and sodium dithionite (3.0 g, 17.46 mmol) was dissolved in EtOH/water (50 mL). The resulting miexture was stirred at 100° C. for overnight. Desired signal was found by LC/MS. After removal of the solvent in vacuo, the residue was purified by flash column chromatography on silica gel (eluting with EA/PE from 0 to 100%) to give tert-butyl 2-(7-methoxy-5-methoxycarbonyl-1-methyl-benzimidazol-2-yl)-1,9-diazatricyclo [6.3.1.04,12]dodeca-2,4(12), 5,7-tetraene-9-carboxylate (4 g, 8.39 mmol, 96.14% yield) as liquid oil. Chemical Formula: C₂₆H₂₈N₄O₅. LC/MS(ESI⁺) [(M+H)⁺]: 476.8.

### Step 8:

Tert-butyl 2-(7-methoxy-5-methoxycarbonyl-1-methyl-benzimidazol-2-yl)-1,9-diazatricyclo [6.3.1.04,12]dodeca-2,4(12), 5,7-tetraene-9-carboxylate (4 g, 8.39 mmol) was dissolved in HCl/ethyl acetate (20 mL). After stirring at RT for 3 h, the resulting mixture was filtered and the filter cake was dried in vacuo to afford methyl 2-(1,9-diazatricyclo [6.3.1.04,12]dodeca-2,4(12), 5,7-tetraen-2-yl)-7-methoxy-1-methyl-benzimidazole-5-carboxylate (3 g, 7.97 mmol, 94.95% yield) as a light yellow solid. LC/MS(ESI⁺) [(M+H)⁺]: 376.8.

### Step 9:

A mixture of ethyl 2-(1,9-diazatricyclo[6.3.1.04,12]dodeca-2,4(12), 5,7-tetraen-2-yl)-7-methoxy-1-methyl-benzimidazole-5-carboxylate (200 mg, 531.34 μmol) and 3-bromopropanenitrile (71.2 mg, 43.94 μL) were dissolved in DMF (10 mL). Cs₂CO₃ (518.1 mg, 1.59 mmol) was added, and the mixture was heated at 100° C. for 18 h. The crude reaction mixture was concentrated and the residue was purified by flash column chromatography on silica gel (eluting with EA/PE from 0 to 70%) to give methyl 2-[9-(2-cyanoethyl)-1,9-diazatricyclo[6.3.1.04,12]dodeca-2,4(12), 5,7-tetraen-2-yl]-7-methoxy-1-methyl-benzimidazole-5-carboxylate (50 mg, 116.42 μmol, 21.91% yield, crude) as a yellow solid. LC/MS(ESI⁺) [(M+H)⁺]: 429.7.

### Step 10:

To a solution of methyl 2-[9-(2-cyanoethyl)-1,9-diazatricyclo [6.3.1.04,12]dodeca-2,4(12), 5,7-tetraen-2-yl]-7-methoxy-1-methyl-benzimidazole-5-carboxylate (50 mg, 116.42 μmol) in Methanol (20 mL) was added Raney Nickel, active catalyst (10.0 mg, 116.42 μmol) in water. The reaction mixture was stirred under the atmosphere of hydrogen with H₂ (balloon) at RT for overnight. Then the mixture was filtered, washed with methanol and the filtrate was concentrated in vacuo. The residue was purified by flash column chromatography on silica gel (eluting with MeOH/DCM from 0 to 50%) to give methyl 2-[9-(3-aminopropyl)-1,9-diazatricyclo[6.3.1.04,12]dodeca-2,4(12), 5,7-tetraen-2-yl]-7-methoxy-1-methyl-benzimidazole-5-carboxylate (25 mg, 57.67 μmol, 49.53% yield) as a white solid. LC/MS(ESI⁺) [(M+H)⁺]: 433.8.

### Step 11:

A solution of methyl 2-[9-(3-aminopropyl)-1,9-diazatricyclo[6.3.1.04,12]dodeca-2,4(12), 5,7-tetraen-2-yl]-7-methoxy-1-methyl-benzimidazole-5-carboxylate (25 mg, 57.67 μmol) and DIPEA (22.4 mg, 173.01 μmol, 30.13 μL) in dichloromethane (5 mL) was added dropwise with a solution of acetyl chloride (4.5 mg, 57.67 μmol, 3.51 μL) in dichloromethane (2 mL). After stirring at RT for 1 h, water (1 mL) was added. The layers were separated, and the organic layer was washed with saturated ammonium chloride (10 mL), dried over magnesium sulfate, and concentrated under reduced pressure. The residue was purified by flash column chromatography on silica gel (eluting with MeOH/DCM from 0 to 30%) to give methyl 2-[9-(3-acetamidopropyl)-1,9-diazatricyclo [6.3.1.04,12]dodeca-2,4(12), 5,7-tetraen-2-yl]-7-methoxy-1-methyl-benzimidazole-5-carboxylate (20 mg, 42.06 μmol, 72.93% yield) as colorless oil. LC/MS(ESI⁺) [(M+H)⁺]: 475.8.

### Step 12:

To a solution of methyl 2-[9-(3-acetamidopropyl)-1,9-diazatricyclo [6.3.1.04,12]dodeca-2,4(12), 5,7-tetraen-2-yl]-7-methoxy-1-methyl-benzimidazole-5-carboxylate (10 mg, 21.03 μmol) in THF (1 mL) was added LiOH (aq. 1 N) (105.14 μmol, 2 mL). The reaction mixture was stirred at RT for overnight. The pH was adjusted to be acidic with 2 mol/L HCl. After removal of the solvent in vacuo, the crude product 2-[9-(3-acetamidopropyl)-1,9-diazatricyclo [6.3.1.04,12]dodeca-2,4(12), 5,7-tetraen-2-yl]-7-methoxy-1-methyl-benzimidazole-5-carboxylic acid (9 mg, 19.50 μmol, 92.74% yield) was obtained as a yellow solid. LC/MS(ESI⁺) [(M+H)⁺]: 461.8. The crude product was used in next reaction without further purification.

### Step 13:

A mixture of 2-[9-(3-acetamidopropyl)-1,9-diazatricyclo[6.3.1.04,12]dodeca-2,4(12), 5,7-tetraen-2-yl]-7-methoxy-1-methyl-benzimidazole-5-carboxylic acid (9 mg, 19.50 μmol), tert-butyl N-[(3R,5R)-5-methyl-3-piperidyl]carbamate (4.2 mg, 19.50 μmol), HATU (7.4 mg, 19.50 μmol) and DIPEA (5.0 mg, 39.00 μmol, 6.79 μL) was dissolved in DMF (2 mL). The resulting mixture was stirred at 50° C. for 10 min. The reaction mixture was diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give tert-butyl N-[(3R,5R)-1-[2-[9-(3-acetamidopropyl)-1,9-diazatricyclo[6.3.1.04,12]dodeca-2,4(12), 5,7-tetraen-2-yl]-7-methoxy-1-methyl-benzimidazole-5-carbonyl]-5-fluoro-3-piperidyl]carbamate (10 mg, 15.11 μmol, 77.49% yield) as a yellow oil. LC/MS(ESI⁺) [(M+H)⁺]: 661.7.

### Step 14:

Tert-butyl N-[(3R,5R)-1-[2-[9-(3-acetamidopropyl)-1,9-diazatricyclo[6.3.1.04,12]dodeca-2,4(12), 5,7-tetraen-2-yl]-7-methoxy-1-methyl-benzimidazole-5-carbonyl]-5-fluoro-3-piperidyl]carbamate (10 mg, 15.11 μmol) was dissolved in HCl/EA (3 mL). The resulting solution was stirred at RT for 30 min. After removal of the solvent in vacuo and purified by pre-HPLC, N-[3-[2-[5-[(3R,5R)-3-amino-5-fluoro-piperidine-1-carbonyl]-7-methoxy-1-methyl-benzimidazol-2-yl]-1,9-diazatricyclo[6.3.1.04,12]dodeca-2,4(12), 5,7-tetraen-9-yl]propyl]acetamide (7 mg, 12.46 μmol, 82.48% yield) was obtained as a white solid. LC/MS(ESI⁺) [(M+H)⁺]: 561.8. ¹H NMR (400 MHz, DMSO-d₆) δ 8.24 (s, 1H), 7.90 (d, J=5.8 Hz, 1H), 7.31 (d, J=1.1 Hz, 1H), 7.02-6.90 (m, 3H), 6.85 (d, J=1.2 Hz, 1H), 6.41 (dd, J=7.1, 1.3 Hz, 1H), 5.05-4.76 (m, 2H), 4.63 (t, J=5.0 Hz, 2H), 4.23 (s, 3H), 3.99 (s, 3H), 3.55 (t, J=5.1 Hz, 3H), 3.17 (q, J=6.5 Hz, 3H), 3.03 (d, J=10.8 Hz, 1H), 2.19 (s, 1H), 1.84 (s, 3H), 1.78 (q, J=7.1 Hz, 2H), 1.64-1.48 (m, 1H).

### Example 2 Preparation of (R)-(3-aminopiperidin-1-yl)(2-(2,3-dihydro-1H-pyrrolo[1,2,3-de]quinoxalin-5-yl)-7-methoxy-1-methyl-1H-benzo[d]imidazol-5-yl)methanone

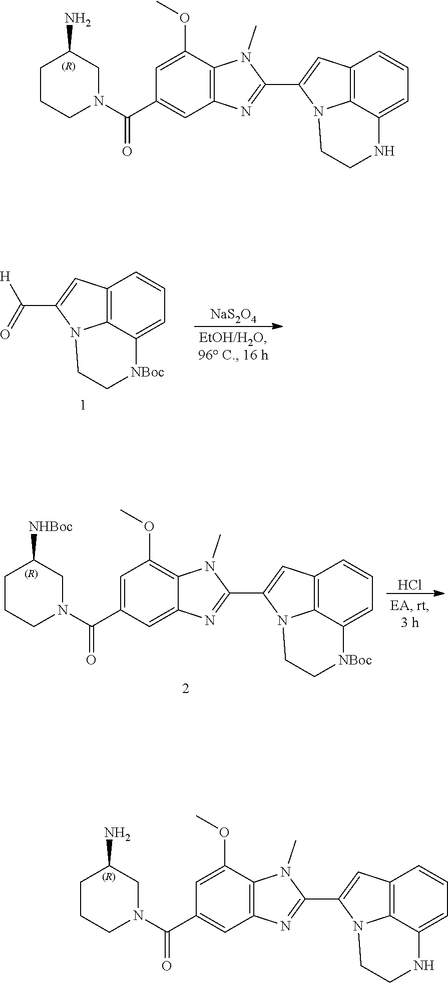

### Step 1:

The suspension of tert-butyl 2-formyl-1,9-diazatricyclo[6.3.1.0^{4,12}]dodeca-2,4(12), 5,7-tetraene-9-carboxylate (24 mg, 83.82 μmol), tert-butyl N-[(3R)-1-[3-methoxy-4-(methylamino)-5-nitro-benzoyl]-3-piperidyl]carbamate (45 mg, 110.17 μmol) and sodium dithionite (43.8 mg, 251.46 μmol) in mixed solvent of EtOH (4 mL) and H₂O (4 mL) was stirred under reflux at 96° C. overnight, cooled and diluted with DCM (40 mL). The organic phase was separated, dried over anhydrous sodium sulfate, and filtered. The filtrate was concentrated in vacuo, and the residue was purified by prep-TLC (DCM/MeOH=15/1) to afford tert-butyl 2-[5-[(3R)-3-(tert-butoxycarbonylamino)piperidine-1-carbonyl]-7-methoxy-1-methyl-benzimidazol-2-yl]-1,9-diazatricyclo[6.3.1.0^{4,12}]dodeca-2,4(12), 5,7-tetraene-9-carboxylate (46 mg, 71.34 μmol, 85.12% yield) as a white solid, LC/MS(ESI⁺) [(M+H)⁺]:644.8.

### Step 2:

To a solution of tert-butyl 2-[5-[(3R)-3-(tert-butoxycarbonylamino)piperidine-1-carbonyl]-7-methoxy-1-methyl-benzimidazol-2-yl]-1,9-diazatricyclo[6.3.1.0^{4,12}]dodeca-2,4(12), 5,7-tetraene-9-carboxylate (46 mg, 71.34 μmol) in ethyl acetate (71.34 μmol, 2 mL) was added hydrogen chloride solution 4.0 M. The resulting mixture was stirred at RT for 3 hours and concentrated in vacuo. The residue was triturated in EA/PE (1/5) and filtered. The solid was dried in vacuo to afford [(3R)-3-amino-1-piperidyl]-[2-(1,9-diazatricyclo[6.3.1.0^{4,12}]dodeca-2,4(12), 5,7-tetraen-2-yl)-7-methoxy-1-methyl-benzimidazol-5-yl]methanone (25 mg, 56.24 μmol) as an off-white solid, LC/MS(ESI⁺) [(M+H)⁺]: 544.8.

### The Following Compounds were Prepared Analogously

### Example 3 Synthesis report of (R)—N-(3-(5-(5-(3-aminopiperidine-1-carbonyl)-7-methoxy-1-methyl-1H-benzo[d]imidazol-2-yl)-2,3-dihydro-1H-pyrrolo[1,2,3-de]quinoxalin-1-yl)propyl)acetamide

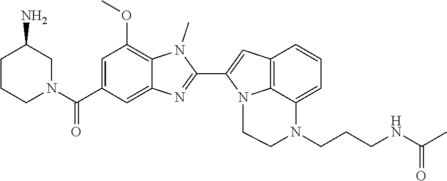 [SMILES: COc1cc(C(=O)N2CCC[C@@H](N)C2)cc2nc(-c3cc4cccc5c4n3CCN5CCCNC(C)=O)n(C)c12]

Prepared in analogous manner as for Example 1. LC/MS(ESI⁺) [(M+H)⁺]: 543.8.

### Example 4 Synthesis report of 1-(3-(5-(5-((3R,5R)-3-amino-5-fluoropiperidine-1-carbonyl)-7-methoxy-1-methyl-1H-benzo[d]imidazol-2-yl)-2,3-dihydro-1H-pyrrolo[1,2,3-de]quinoxalin-1-yl)propyl)-3-cyclopropylurea

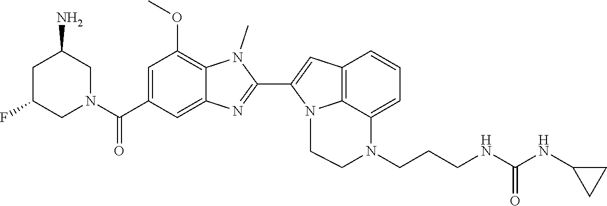 [SMILES: COc1cc(C(=O)N2C[C@H](N)C[C@@H](F)C2)cc2nc(-c3cc4cccc5c4n3CCN5CCCNC(=O)NC3CC3)n(C)c12]

Prepared in analogous manner as for Example 1. LC/MS(ESI⁺) [(M+H)⁺]: 602.8.

### Example 5 Synthesis report of (R)-1-(3-(5-(5-(3-aminopiperidine-1-carbonyl)-7-methoxy-1-methyl-1H-benzo[d]imidazol-2-yl)-2,3-dihydro-1H-pyrrolo[1,2,3-de]quinoxalin-1-yl)propyl)-3-cyclopropylurea

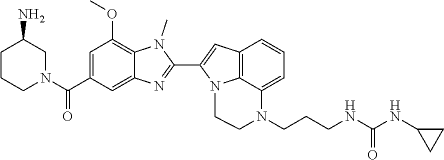 [SMILES: COc1cc(C(=O)N2CCC[C@@H](N)C2)cc2nc(-c3cc4cccc5c4n3CCN5CCCNC(=O)NC3CC3)n(C)c12]

Prepared in analogous manner as for Example 1. LC/MS(ESI⁺) [(M+H)⁺]: 584.8.

### Example 6 Synthesis report of 1-(3-(5-(5-((1R,4R,7R)-7-amino-2-azabicyclo[2.2.1]heptane-2-carbonyl)-7-methoxy-1-methyl-1H-benzo[d]imidazol-2-yl)-2,3-dihydro-1H-pyrrolo[1,2,3-de]quinoxalin-1-yl)propyl)-3-cyclopropylurea

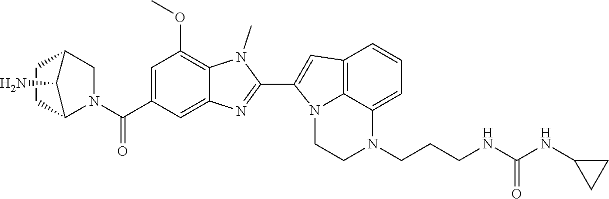 [SMILES: COc1cc(C(=O)N2C[C@H]3CC[C@@H]2[C@@H]3N)cc2nc(-c3cc4cccc5c4n3CCN5CCCNC(=O)NC3CC3)n(C)c12]

Prepared in analogous manner as for Example 1. LC/MS(ESI⁺) [(M+H)⁺]: 596.8. ¹H NMR (400 MHz, DMSO) δ 8.19 (s, 1H), 7.46 (s, 1H), 7.35 (s, 1H), 7.01-6.92 (m, 4H), 6.40 (d, J=7.0 Hz, 1H), 6.10 (d, J=2.6 Hz, 1H), 5.96 (t, J=5.8 Hz, 1H), 4.63 (s, 2H), 4.22 (d, J=3.1 Hz, 3H), 4.00 (s, 3H), 3.78 (s, 1H), 3.55 (t, J=5.1 Hz, 3H), 3.21-2.99 (m, 6H), 2.42 (dt, J=6.8, 3.4 Hz, 1H), 2.23 (s, 1H), 1.98 (t, J=12.3 Hz, 2H), 1.77 (dt, J=15.9, 8.2 Hz, 3H), 1.45 (s, 1H), 0.60-0.55 (m, 2H), 0.37-0.33 (m, 2H).

### Example 7 Synthesis report of (R)-1-(3-(5-(5-(3-aminopiperidine-1-carbonyl)-7-methoxy-1-methyl-1H-benzo[d]imidazol-2-yl)-2,3-dihydro-1H-pyrrolo[1,2,3-de]quinoxalin-1-yl)propyl)-3-cyclopropylurea

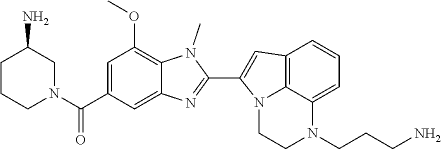 [SMILES: COc1cc(C(=O)N2CCC[C@@H](N)C2)cc2nc(-c3cc4cccc5c4n3CCN5CCCN)n(C)c12]

Prepared in analogous manner as for Example 1. LC/MS(ESI⁺) [(M+H)⁺]: 501.8. ¹H NMR (400 MHz, DMSO) δ 8.37 (s, 2H), 7.30 (d, J=1.2 Hz, 1H), 7.02-6.93 (m, 3H), 6.86 (d, J=1.2 Hz, 1H), 6.47 (d, J=7.1 Hz, 1H), 4.64 (t, J=5.2 Hz, 2H), 4.23 (s, 3H), 4.00 (s, 3H), 2.87 (t, J=7.3 Hz, 2H), 2.78 (d, J=7.5 Hz, 2H), 1.91 (t, J=7.5 Hz, 3H), 1.71 (s, 1H), 1.55-1.26 (m, 3H).

### Example 8 Preparation of (R)-6-(5-(3-aminopiperidine-1-carbonyl)-7-methoxy-1-methyl-1H-benzo[d]imidazol-2-yl)-3,4-dihydro-[1,4]diazepino[3,2,1-hi]indol-2(1H)-one

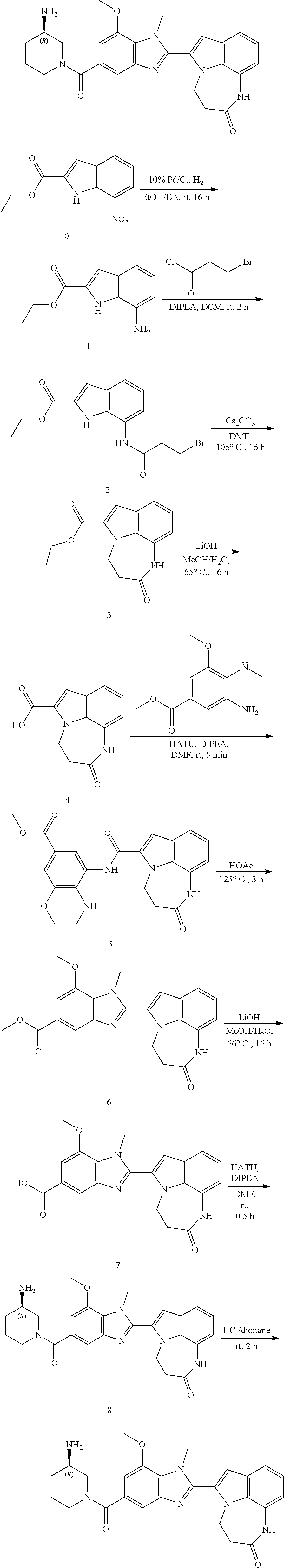

### Step 1:

To a solution of ethyl 7-nitro-1H-indole-2-carboxylate (1.08 g, 4.61 mmol) in ethanol (30 mL) and ethyl acetate (30 mL) was added palladium on carbon (100 mg, 939.67 μmol) at RT. The reaction mixture was stirred at RT under H₂ atmosphere for 16 h and filtered. The filtrate was concentrated in vacuo to afford ethyl 7-amino-1H-indole-2-carboxylate (940 mg, 4.60 mmol, 99.82% yield) as a light-yellow solid. LC/MS (ESI⁺) [(M+H)⁺]: 205.

### Step 2:

To a solution of ethyl 7-amino-1H-indole-2-carboxylate (100 mg, 489.66 μmol) in DCM (2 mL) were added 3-bromopropanoyl chloride (167.87 mg, 979.31 μmol) and N-ethyl-N-isopropyl-propan-2-amine (316.42 mg, 2.45 mmol, 426.44 μL) subsequently at RT. The reaction mixture was stirred at RT for 2 h, diluted with DCM (10 mL) and quenched with water (10 mL). The two phases were separated, and the aqueous phase was extracted with DCM (10 mL*3). The combined organic phase was dried over anhydrous sodium sulfate and filtered. The filtrate was concentrated in vacuo to provide crude ethyl 7-(3-bromopropanamido)-1H-indole-2-carboxylate (160 mg, 471.72 μmol, 96.34% yield). LC/MS (ESI⁺) [(M+H)⁺]: 340.

### Step 3:

To a solution of ethyl 7-(3-bromopropanamido)-1H-indole-2-carboxylate (160 mg, 471.72 μmol) in DMF (3 mL) was added dicesium carbonate (460.0 mg, 1.42 mmol) at RT. The reaction mixture was stirred at 106° C. for 16 h. The mixture was quenched with ice H₂O (10 mL) and extracted with ethyl acetate (30 mL×3). The organic phase was washed with brine (5 mL×3) and dried over anhydrous sodium sulfate and filtered. The filtrate was concentrated in vacuo, and the residue was purified by flash column chromatography on silica gel (PE/EA=1/0-1/1) to provide ethyl 10-oxo-1,9-diazatricyclo[6.4.1.04,13]trideca-2,4(13), 5,7-tetraene-2-carboxylate (80 mg, 308.88 μmol, 65.48% yield) as a white solid. LC/MS (ESI⁺) [(M+H)⁺]: 259.

### Step 4:

The mixture of ethyl 10-oxo-1,9-diazatricyclo[6.4.1.0^{4,13}]trideca-2,4(13), 5,7-tetraene-2-carboxylate (80 mg, 309.75 μmol) and lithium hydroxide (7.4 mg, 309.75 μmol) in a mixed solvent of methanol (5 mL) and water (1 mL) was stirred at 65° C. for 16 h and concentrated in vacuo. The residue was acidified with 2N HCl aqueous solution to pH ˜5 and filtered to afford 10-oxo-1,9-diazatricyclo[6.4.1.0^{4,13}]trideca-2,4(13), 5,7-tetraene-2-carboxylic acid (60 mg, 260.62 μmol, 84.14% yield) as a white solid. LC/MS (ESI⁺) [(M+H)⁺]: 231.

### Step 5:

To a solution of 10-oxo-1,9-diazatricyclo[6.4.1.0^{4,13}]trideca-2,4(13), 5,7-tetraene-2-carboxylic acid (23 mg, 99.90 μmol), methyl 3-amino-5-methoxy-4-(methylamino)benzoate (73.0 mg, 347.24 μmol) and [dimethylamino(triazolo[4,5-b]pyridin-3-yloxy)methylene]-dimethyl-ammonium; hexafluorophosphate (42 mg, 110.46 μmol) in DMF (2 mL) was added N-ethyl-N-isopropyl-propan-2-amine (64.6 mg, 499.52 μmol, 87.01 μL) at RT. The reaction mixture was stirred at RT for 5 minutes and purified by reverse phase chromatography to afford methyl 3-methoxy-4-(methylamino)-5-[(10-oxo-1,9-diazatricyclo[6.4.1.0^{4,13}]trideca-2,4(13), 5,7-tetraene-2-carbonyl)amino]benzoate (29 mg, 68.65 μmol, 68.72% yield) and its isomer as a white solid, LC/MS (ESI⁺) [(M+H)⁺]: 423.

### Step 6:

The mixture of methyl 3-methoxy-4-(methylamino)-5-[(10-oxo-1,9-diazatricyclo[6.4.1.0^{4,13}]trideca-2,4(13), 5,7-tetraene-2-carbonyl)amino]benzoate (29 mg, 68.65 μmol) in HOAc (5 mL) was stirred at 125° C. for 3 h. After cooling to RT, the resulting mixture was concentrated in vacuo to afford a crude methyl 7-methoxy-1-methyl-2-(10-oxo-1,9-diazatricyclo[6.4.1.0^{4,13}]trideca-2,4(13), 5,7-tetraen-2-yl)benzimidazole-5-carboxylate (27 mg, 66.76 μmol, 97.25% yield) as a white solid. LC/MS (ESI⁺) [(M+H)⁺]: 405.

### Step 7:

To a mixture of methyl 7-methoxy-1-methyl-2-(10-oxo-1,9-diazatricyclo[6.4.1.0^{4,13}]trideca-2,4(13), 5,7-tetraen-2-yl)benzimidazole-5-carboxylate (25 mg, 61.82 μmol) in methanol (10 mL) and water (1 mL) was added lithium hydroxide hydrate (13.0 mg, 309.09 μmol, 8.59 μL), the resulting mixture was stirred at 60° C. for 16 h, cooled and concentrated in vacuo. The residue was acidified with 2N HCl aqueous solution to pH ˜5 and filtered to afford 7-methoxy-1-methyl-2-(10-oxo-1,9-diazatricyclo[6.4.1.0^{4,13}]trideca-2,4(13), 5,7-tetraen-2-yl)benzimidazole-5-carboxylic acid (20 mg, 51.23 μmol, 82.9% yield) as a white solid. LC/MS (ESI⁺) [(M+H)⁺]: 391.

### Step 8:

To a solution of 7-methoxy-1-methyl-2-(10-oxo-1,9-diazatricyclo[6.4.1.0^{4,13}]trideca-2,4(13), 5,7-tetraen-2-yl)benzimidazole-5-carboxylic acid (10 mg, 25.62 μmol), tert-butyl N-[(3R)-3-piperidyl]carbamate (6.2 mg, 30.74 μmol) and [dimethylamino(triazolo[4,5-b]pyridin-3-yloxy)methylene]-dimethyl-ammonium; hexafluorophosphate (48.7 mg, 128.08 μmol) in DMF (2 mL) was added N-ethyl-N-isopropyl-propan-2-amine (3.6 mg, 28.18 μmol, 4.91 μL) at RT. The reaction mixture was stirred at RT for 0.5 h and purified by prep-HPLC to afford tert-butyl N-[(3R)-1-[7-methoxy-1-methyl-2-(10-oxo-1,9-diazatricyclo[6.4.1.0^{4,13}]trideca-2,4(13), 5,7-tetraen-2-yl)benzimidazole-5-carbonyl]-3-piperidyl]carbamate (10 mg, 17.46 μmol, 68.17% yield) as a yellow solid. LC/MS (ESI⁺) [(M+H)⁺]: 573.

### Step 9:

To a solution of tert-butyl N-[(3R)-1-[7-methoxy-1-methyl-2-(10-oxo-1,9-diazatricyclo[6.4.1.0^{4,13}]trideca-2,4(13), 5,7-tetraen-2-yl)benzimidazole-5-carbonyl]-3-piperidyl]carbamate (10 mg, 17.46 μmol) in dioxane (2 mL) was added Hydrogen chloride solution 4.0 M in dioxane (2 mL) at RT. The mixture was stirred at rt for 2 h and concentrated in vacuo, and residue was triturated in EA/PE (1/10, 11 mL). The white solid was collected by filtration to afford 2-[5-[(3R)-3-aminopiperidine-1-carbonyl]-7-methoxy-1-methyl-benzimidazol-2-yl]-1,9-diazatricyclo[6.4.1.0^{4,13}]trideca-2,4(13), 5,7-tetraen-10-one (8 mg, 16.93 μmol, 96.95% yield). LC/MS (ESI⁺) [(M+H)⁺]: 473.

### The Following Compounds were Prepared Analogously

### Example 9 (R)-5-(5-(3-aminopiperidine-1-carbonyl)-7-methoxy-1-methyl-1H-benzo[d]imidazol-2-yl)-1H-pyrrolo[1,2,3-de]quinoxalin-2(3H)-one

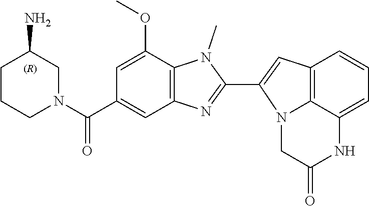

Prepared in analogous manner as for Example 8. LC/MS(ESI⁺) [(M+H)⁺]: 458.8.

¹H NMR (400 MHz, DMSO-d₆) δ 8.29 (d, J=3.3 Hz, 1H), 7.36 (s, 1H), 7.28 (d, J=8.0 Hz, 1H), 7.19 (s, 1H), 7.05-6.99 (m, 1H), 6.88 (s, 1H), 6.68 (d, J=7.3 Hz, 1H), 5.32 (s, 2H), 4.27 (s, 3H), 4.00 (s, 3H), 3.00 (s, 3H), 2.82 (s, 2H), 1.91 (s, 1H), 1.71 (s, 1H), 1.50 (s, 1H), 1.34 (s, 1H).

### Example 10 Synthesis report of (R)-(3-aminopiperidin-1-yl)(2-(1-(2-hydroxyethyl)-2,3-dihydro-1H-pyrrolo[1,2,3-de]quinoxalin-5-yl)-7-methoxy-1-methyl-1H-benzo[d]imidazol-5-yl)methanone

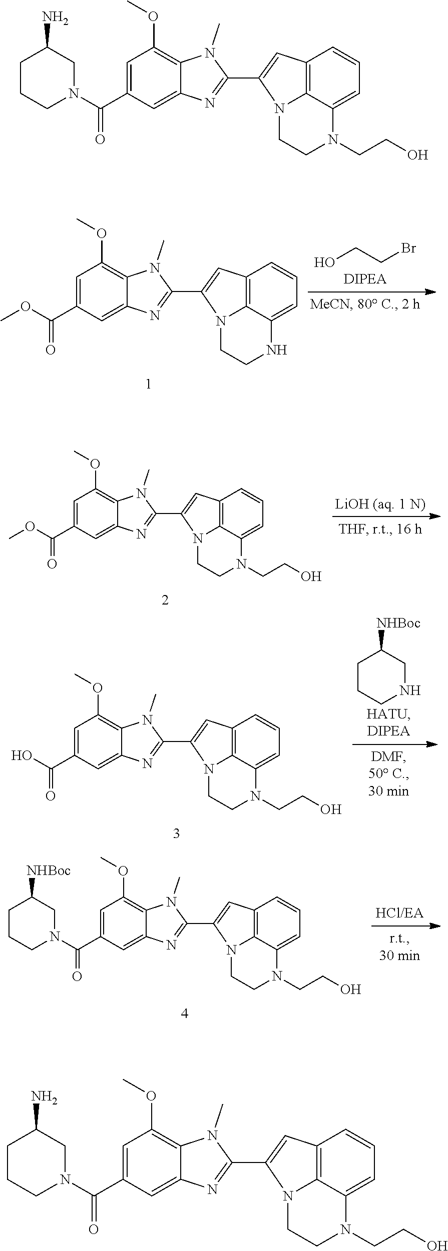

### Step 1:

To a solution of methyl 2-(1,9-diazatricyclo[6.3.1.04,12]dodeca-2,4(12), 5,7-tetraen-2-yl)-7-methoxy-1-methyl-benzimidazole-5-carboxylate (20.0 mg, 53.13 μmol) in Acetonitrile (5 mL) and DIPEA (13.7 mg, 106.26 μmol, 18.5 μL) was added 2-bromoethanol (6.6 mg, 53.1 μmol) at RT, and the reaction mixture was heated at 80° C. by microwave for 2 h. The solution was purified by pre-HPLC to afford methyl 2-[9-(2-hydroxyethyl)-1,9-diazatricyclo[6.3.1.04,12]dodeca-2,4(12), 5,7-tetraen-2-yl]-7-methoxy-1-methyl-benzimidazole-5-carboxylate (15.0 mg, 35.68 μmol, 67.14% yield). LC/MS (ESI⁺) [(M+H)⁺]: 420.8.

### Step 2:

To a solution of methyl 2-[9-(2-hydroxyethyl)-1,9-diazatricyclo[6.3.1.04,12]dodeca-2,4(12), 5,7-tetraen-2-yl]-7-methoxy-1-methyl-benzimidazole-5-carboxylate (15.0 mg, 35.68 μmol) in THF (2 mL) was added LiOH (aq.1 N) (76.40 μmol, 5 mL). The resulting mixture was stirred at RT for overnight. The pH of the reaction solution was adjusted to be <7.0 with 2 mol/L HCl. After removal of the solvent in vacuo, the crude product 2-[9-(2-hydroxyethyl)-1,9-diazatricyclo[6.3.1.04,12]dodeca-2,4(12), 5,7-tetraen-2-yl]-7-methoxy-1-methyl-benzimidazole-5-carboxylic acid (12 mg, 29.53 μmol, 82.8% yield) was obtained as a yellow solid. LC/MS(ESI⁺) [(M+H)⁺]: 406.8. The crude product was used in next step reaction without further purification.

### Step 3:

A mixture of 2-[9-(2-hydroxyethyl)-1,9-diazatricyclo[6.3.1.04,12]dodeca-2,4(12), 5,7-tetraen-2-yl]-7-methoxy-1-methyl-benzimidazole-5-carboxylic acid (12 mg, 29.53 μmol), tert-butyl N-[(3R)-3-piperidyl]carbamate (5.9 mg, 29.53 μmol), HATU (11.2 mg, 29.53 μmol) and DIPEA (11.5 mg, 88.58 μmol, 15.43 μL) was dissolved in DMF (5 mL). The resulting mixture was stirred at 50° C. for 30 min. The reaction was diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give tert-butyl N-[(3R)-1-[2-[9-(2-hydroxyethyl)-1,9-diazatricyclo[6.3.1.04,12]dodeca-2,4(12), 5,7-tetraen-2-yl]-7-methoxy-1-methyl-benzimidazole-5-carbonyl]-3-piperidyl]carbamate (10 mg, 16.99 μmol, 57.53% yield) as yellow oil. LC/MS(ESI⁺) [(M+H)⁺]: 588.8.

### Step 4:

Tert-butyl N-[(3R)-1-[2-[9-(2-hydroxyethyl)-1,9-diazatricyclo[6.3.1.04,12]dodeca-2,4(12), 5,7-tetraen-2-yl]-7-methoxy-1-methyl-benzimidazole-5-carbonyl]-3-piperidyl]carbamate (10 mg, 16.99 μmol) was dissolved in HCl/ethyl acetate (5 mL). The resulting mixture was stirred at RT for 30 min. After removal of the solvent in vacuo, the residue was purified by pre-HPLC to afford [(3R)-3-amino-1-piperidyl]-[2-[9-(2-hydroxyethyl)-1,9-diazatricyclo[6.3.1.04,12]dodeca-2,4(12), 5,7-tetraen-2-yl]-7-methoxy-1-methyl-benzimidazol-5-yl]methanone (5 mg, 10.23 μmol, 60.25% yield) as a light yellow solid. LC/MS (ESI⁺) [(M+H)⁺]: 488.8.

### The Following Compounds were Prepared Analogously

### Example 11 Synthesis report of ((1R,4R,7R)-7-amino-2-azabicyclo[2.2.1]heptan-2-yl)(2-(1-(2-hydroxyethyl)-2,3-dihydro-1H-pyrrolo[1,2,3-de]quinoxalin-5-yl)-7-methoxy-1-methyl-1H-benzo[d]imidazol-5-yl)methanone

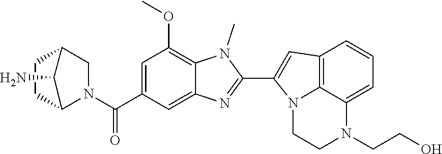 [SMILES: COc1cc(C(=O)N2C[C@H]3CC[C@@H]2[C@@H]3N)cc2nc(-c3cc4cccc5c4n3CCN5CCO)n(C)c12]

Prepared in analogous manner as for Example 10. LC/MS(ESI⁺) [(M+H)⁺]: 500.8.

### Example 12 Synthesis report of (R)-(3-aminopiperidin-1-yl)(2-(1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone

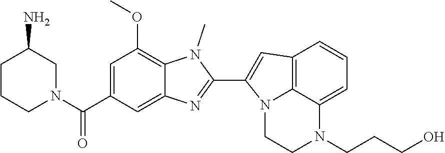 [SMILES: COc1cc(C(=O)N2CCC[C@@H](N)C2)cc2nc(-c3cc4cccc5c4n3CCN5CCCO)n(C)c12]

Prepared in analogous manner as for Example 10. LC/MS(ESI⁺) [(M+H)⁺]: 502.8.

### Example 13 Synthesis report of ((3R,5R)-3-amino-5-fluoropiperidin-1-yl)(2-(1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone)

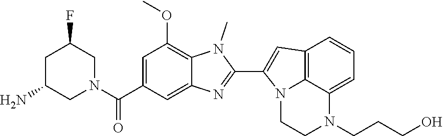 [SMILES: COc1cc(C(=O)N2C[C@H](N)C[C@@H](F)C2)cc2nc(-c3cc4cccc5c4n3CCN5CCCO)n(C)c12]

Prepared in analogous manner as for Example 10. LC/MS(ESI⁺) [(M+H)⁺]: 520.7.

### Example 14 Synthesis report of ((1R,4R,7R)-7-amino-2-azabicyclo[2.2.1]heptan-2-yl)(2-(1-(2-hydroxyethyl)-2,3-dihydro-1H-pyrrolo[1,2,3-de]quinoxalin-5-yl)-7-methoxy-1-methyl-1H-benzo[d]imidazol-5-yl)methanone

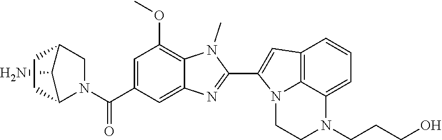 [SMILES: COc1cc(C(=O)N2C[C@H]3CC[C@@H]2[C@@H]3N)cc2nc(-c3cc4cccc5c4n3CCN5CCCO)n(C)c12]

Prepared in analogous manner as for Example 10. LC/MS(ESI⁺) [(M+H)⁺]: 614.8.

### Example 15 (R)-(3-aminopyrrolidin-1-yl)(2-(1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone

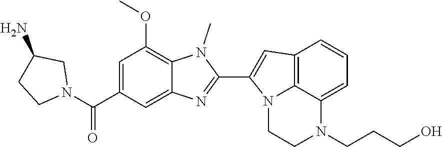 [SMILES: COc1cc(C(=O)N2CC[C@@H](N)C2)cc2nc(-c3cc4cccc5c4n3CCN5CCCO)n(C)c12]

Prepared in analogous manner as for Example 10. LC/MS(ESI⁺) [(M+H)⁺]: 488.8.

### Example 16 (R)-(3-amino-3-methylpiperidin-1-yl)(2-(1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone

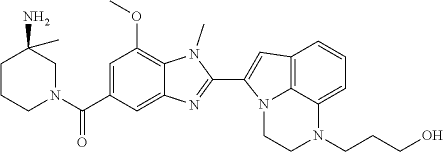 [SMILES: COc1cc(C(=O)N2CCC[C@@](C)(N)C2)cc2nc(-c3cc4cccc5c4n3CCN5CCCO)n(C)c12]

Prepared in analogous manner as for Example 10. LC/MS(ESI⁺) [(M+H)⁺]: 516.8.

### Example 17 ((3R,5R)-3-amino-5-(trifluoromethyl)piperidin-1-yl)(2-(1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone

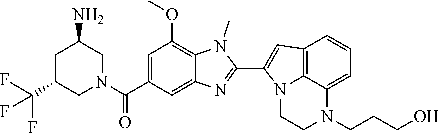 [SMILES: COc1cc(C(=O)N2C[C@H](N)C[C@@H](C(F)(F)F)C2)cc2nc(-c3cc4cccc5c4n3CCN5CCCO)n(C)c12]

Prepared in analogous manner as for Example 10. LC/MS(ESI⁺) [(M+H)⁺]: 570.8.

### Example 18 (R)-(5-amino-3,3-difluoropiperidin-1-yl)(2-(1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone

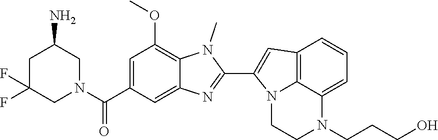 [SMILES: COc1cc(C(=O)N2C[C@H](N)CC(F)(F)C2)cc2nc(-c3cc4cccc5c4n3CCN5CCCO)n(C)c12]

Prepared in analogous manner as for Example 10. LC/MS(ESI⁺) [(M+H)⁺]: 538.8.

### Example 19 (S)-(3-amino-3-methylpiperidin-1-yl)(2-(1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone

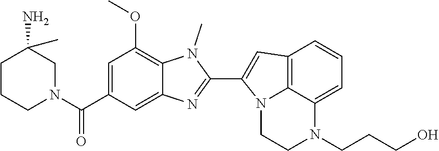 [SMILES: COc1cc(C(=O)N2CCC[C@](C)(N)C2)cc2nc(-c3cc4cccc5c4n3CCN5CCCO)n(C)c12]

Prepared in analogous manner as for Example 10. LC/MS(ESI⁺) [(M+H)⁺]: 516.8.

### Example 20 ((3S,4R)-3-amino-4-fluoropiperidin-1-yl)(2-(1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone

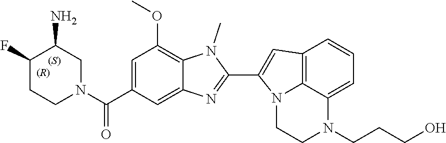

Prepared in analogous manner as for Example 10. LC/MS(ESI⁺) [(M+H)⁺]: 520.8

### Example 21 Synthesis report of (R)-(3-aminopiperidin-1-yl)(7-methoxy-2-(1-(3-methoxypropyl)-2,3-dihydro-1H-pyrrolo[1,2,3-de]quinoxalin-5-yl)-1-methyl-1H-benzo[d]imidazol-5-yl)methanone

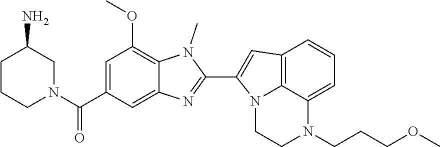 [SMILES: COCCCN1CCn2c(-c3nc4cc(C(=O)N5CCC[C@@H](N)C5)cc(OC)c4n3C)cc3cccc1c32]

Prepared in analogous manner as for Example 10. LC/MS(ESI⁺) [(M+H)⁺]: 514.9.

### Example 22 ((3R,5R)-3-amino-5-fluoropiperidin-1-yl)(2-(1-(2-(2-hydroxyethoxy)ethyl)-2,3-dihydro-1H-pyrrolo[1,2,3-de]quinoxalin-5-yl)-7-methoxy-1-methyl-1H-benzo[d]imidazol-5-yl)methanone

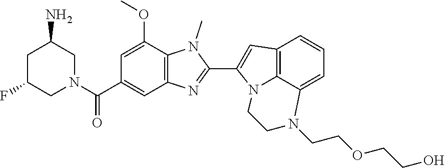 [SMILES: COc1cc(C(=O)N2C[C@H](N)C[C@@H](F)C2)cc2nc(-c3cc4cccc5c4n3CCN5CCOCCO)n(C)c12]

Prepared in analogous manner as for Example 10. LC/MS(ESI⁺) [(M+H)⁺]: 550.8

### Example 23 ((3R,5R)-3-amino-5-fluoropiperidin-1-yl)(2-(1-(3-hydroxypropyl)-2,3-dihydro-1H-pyrrolo[1,2,3-de]quinoxalin-5-yl)-7-methoxy-1-(2,2,2-trifluoroethyl)-1H-benzo[d]imidazol-5-yl)methanone

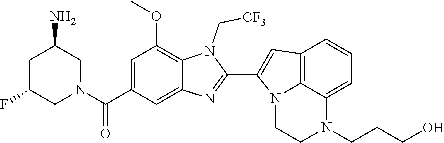 [SMILES: COc1cc(C(=O)N2C[C@H](N)C[C@@H](F)C2)cc2nc(-c3cc4cccc5c4n3CCN5CCCO)n(CC(F)(F)F)c12]

Prepared in analogous manner as for Example 10. LC/MS(ESI⁺) [(M+H)⁺]: 588.8.

¹H NMR (400 MHz, DMSO-d₆) δ 7.38 (s, 1H), 7.02 (s, 1H), 7.00-6.91 (m, 3H), 6.44 (dd, J=6.4, 1.9 Hz, 1H), 5.54 (q, J=8.6 Hz, 2H), 5.07-4.77 (m, 2H), 4.44 (t, J=4.9 Hz, 4H), 4.02 (s, 3H), 3.55 (t, J=5.8 Hz, 4H), 3.49-3.41 (m, 4H), 3.03 (t, J=10.9 Hz, 1H), 2.18 (s, 1H), 1.85-1.76 (m, 2H), 1.64-1.48 (m, 1H).

### Example 24 Preparation of (R)-(3-aminopiperidin-1-yl)(2-(1-benzyl-2,3-dihydro-1H-pyrrolo[1,2,3-de]quinoxalin-5-yl)-7-methoxy-1-methyl-1H-benzo[d]imidazol-5-yl)methanone

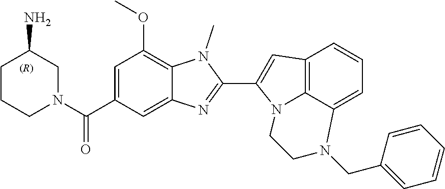

Prepared in analogous manner as for Example 10. LC/MS (ESI⁺) [(M+H)⁺]: 535.

### Example 25 Preparation of ((R)-(3-aminopiperidin-1-yl)(7-methoxy-1-methyl-2-(1-methyl-2,3-dihydro-1H-pyrrolo[1,2,3-de]quinoxalin-5-yl)-1H-benzo[d]imidazol-5-yl)methanone

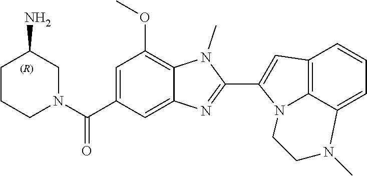

Prepared in analogous manner as for Example 10. LC/MS (ESI⁺) [(M+H)⁺]: 459.

### Example 26 ((3R,5R)-3-amino-5-fluoropiperidin-1-yl)(7-methoxy-1-methyl-2-(1-((1-methyl-1H-pyrazol-4-yl)methyl)-2,3-dihydro-1H-pyrrolo[1,2,3-de]quinoxalin-5-yl)-1H-benzo[d]imidazol-5-yl)methanone

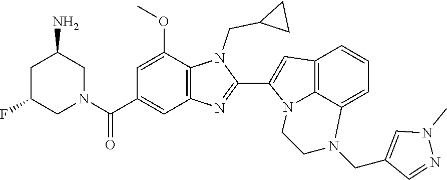 [SMILES: COc1cc(C(=O)N2C[C@H](N)C[C@@H](F)C2)cc2nc(-c3cc4cccc5c4n3CCN5Cc3cnn(C)c3)n(CC3CC3)c12]

Prepared in analogous manner as for Example 10. LC/MS(ESI⁺) [(M+H)⁺]: 556.8.

¹H NMR (400 MHz, DMSO-d₆) δ 7.69 (s, 1H), 7.44 (s, 1H), 7.29 (d, J=1.2 Hz, 1H), 7.02-6.95 (m, 3H), 6.84 (s, 1H), 6.60 (d, J=7.2 Hz, 1H), 4.65-4.61 (m, 2H), 4.44 (s, 2H), 4.21 (s, 3H), 3.99 (s, 3H), 3.79 (s, 3H), 3.50 (t, J=5.2 Hz, 2H), 3.01 (s, 3H), 2.16 (s, 2H), 1.48 (s, 2H).

### Example 27 Synthesis report of (R)-3-(5-(5-(3-aminopiperidine-1-carbonyl)-7-methoxy-1-methyl-1H-benzo[d]imidazol-2-yl)-2,3-dihydro-1H-pyrrolo[1,2,3-de]quinoxalin-1-yl)propyl cyclopropylcarbamate

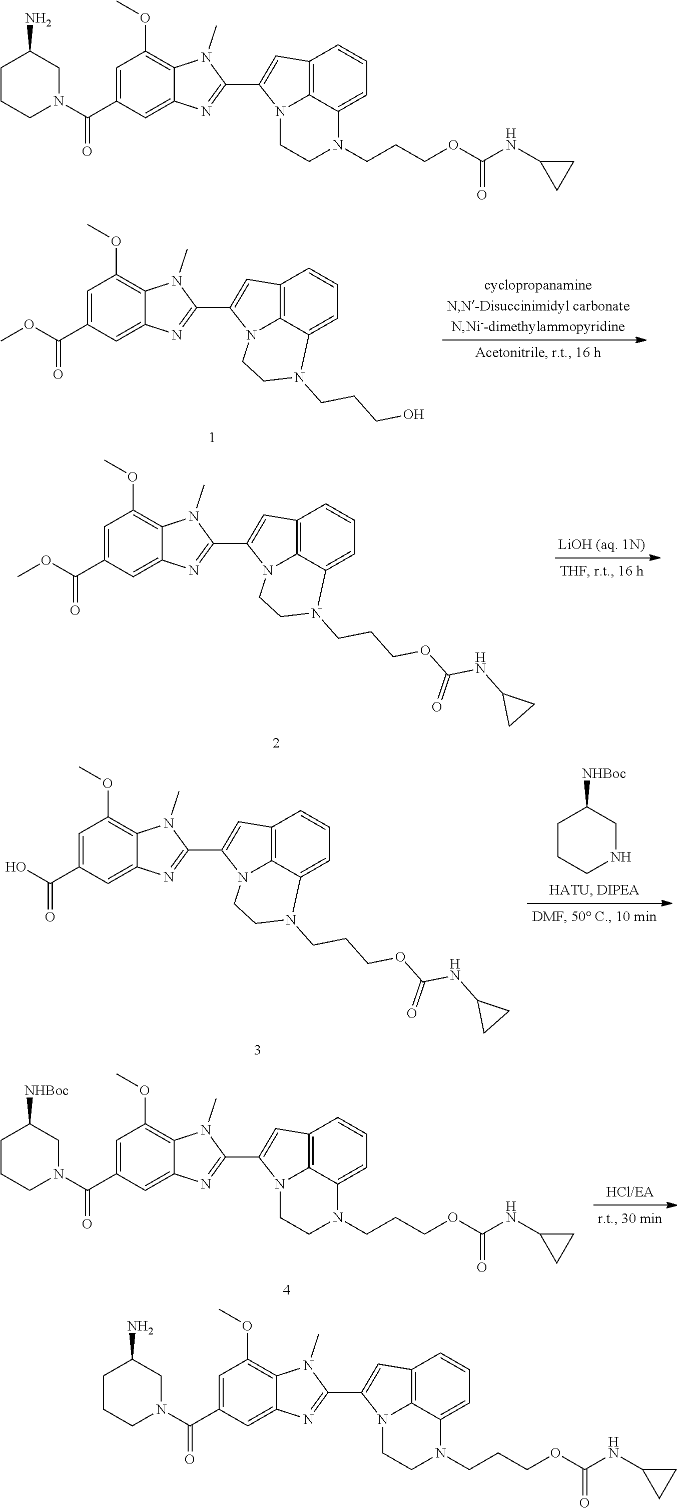

### Step 1:

Methyl 2-[9-(3-hydroxypropyl)-1,9-diazatricyclo[6.3.1.04,12]dodeca-2,4(12), 5,7-tetraen-2-yl]-7-methoxy-1-methyl-benzimidazole-5-carboxylate (50 mg, 115.08 μmol) was dissolved in acetonitrile (10 mL), and then thereto were added N,N′-disuccinimidyl carbonate (44.2 mg, 172.62 μmol) and N,N′-dimethylammopyridine (14.1 mg, 115.08 μmol), and the mixture was stirred at RT for 16 h. Then, thereto was added cyclopropanamine (13.2 mg, 230.16 μmol, 15.95 μL), and the mixture was stirred at RT for 1 h. To the reaction mixture was added brine, and the mixture was extracted with ethyl acetate. The extracted layer was washed with saturated saline, and dried over anhydrous sodium sulfate, and then the solvent was distilled away under reduced pressure. The residue was purified by silica gel column chromatography (eluent ethyl acetate: PE 0:100 to 80:20 gradient) to Methyl 2-[9-[3-(cyclopropylcarbamoyloxy)propyl]-1,9-diazatricyclo[6.3.1.04,12]dodeca-2,4(12), 5,7-tetraen-2-yl]-7-methoxy-1-methyl-benzimidazole-5-carboxylate.

Step 2: Methyl 2-[9-[3-(cyclopropylcarbamoyloxy)propyl]-1,9-diazatricyclo[6.3.1.04,12]dodeca-2,4(12), 5,7-tetraen-2-yl]-7-methoxy-1-methyl-benzimidazole-5-carboxylate (20 mg, 38.64 μmol) was dissolved in THF (1 mL). LiOH (aq. 1 N) (38.64 μmol, 2 mL) was added into the solution. The reaction mixture was stirred at RT for overnight. the pH of the reaction mixture was adjusted to be acidic with 2 mol/L HCl. After removal of the solvent in vacuo, the crude product 2-[9-[3-(cyclopropylcarbamoyloxy)propyl]-1,9-diazatricyclo[6.3.1.04,12]dodeca-2,4(12), 5,7-tetraen-2-yl]-7-methoxy-1-methyl-benzimidazole-5-carboxylic acid (15 mg, 29.79 μmol, 77.09% yield) was obtained as a yellow solid. The crude product was used in next reaction without further purification. LC/MS(ESI⁺) [(M+H)⁺]: 503.8.

### Step 3:

A mixture of 2-[9-[3-(cyclopropylcarbamoyloxy)propyl]-1,9-diazatricyclo[6.3.1.04,12]dodeca-2,4(12), 5,7-tetraen-2-yl]-7-methoxy-1-methyl-benzimidazole-5-carboxylic acid (15 mg, 29.79 μmol), tert-butyl N-[(3R)-3-piperidyl]carbamate (6.0 mg, 29.79 μmol), HATU (11.3 mg, 29.79 μmol) and DIPEA (11.6 mg, 89.37 μmol, 15.57 μL) was dissolved in DMF (3 mL). The resulting mixture was stirred at 50° C. for 10 min, diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give 3-[2-[5-[(3R)-3-(tert-butoxycarbonylamino)piperidine-1-carbonyl]-7-methoxy-1-methyl-benzimidazol-2-yl]-1,9-diazatricyclo[6.3.1.04,12]dodeca-2,4(12), 5,7-tetraen-9-yl]propyl N-cyclopropylcarbamate (15 mg, 21.87 μmol, 73.42% yield) as yellow oil. LC/MS (ESI⁺) [(M+H)⁺]: 685.8.

### Step 4:

3-[2-[5-[(3R)-3-(tert-butoxycarbonylamino)piperidine-1-carbonyl]-7-methoxy-1-methyl-benzimidazol-2-yl]-1,9-diazatricyclo[6.3.1.04,12]dodeca-2,4(12), 5,7-tetraen-9-yl]propyl N-cyclopropylcarbamate (15 mg, 21.87 μmol) was dissolved in HCl/EA (2 mL). The reaction mixture was stirred at RT for 30 min. After removal of the solvent in vacuo, the residue was purified by pre-HPLC to afford 3-[2-[5-[(3R)-3-aminopiperidine-1-carbonyl]-7-methoxy-1-methyl-benzimidazol-2-yl]-1,9-diazatricyclo[6.3.1.04,12]dodeca-2,4(12), 5,7-tetraen-9-yl]propyl N-cyclopropylcarbamate (10 mg, 17.07 μmol, 78.06% yield) as a white solid. LC/MS (ESI⁺) [(M+H)⁺]: 585.8.

¹H NMR (400 MHz, DMSO-d₆) δ 8.26 (s, 1H), 7.33 (s, 2H), 7.01-6.93 (m, 3H), 6.87 (d, J=1.2 Hz, 1H), 6.42 (d, J=7.1 Hz, 1H), 4.63 (t, J=5.1 Hz, 2H), 4.23 (s, 3H), 4.07 (d, J=6.8 Hz, 2H), 4.00 (s, 3H), 3.56 (d, J=5.3 Hz, 4H), 3.01 (s, 2H), 2.88 (d, J=9.5 Hz, 3H), 1.94 (s, 3H), 1.72 (s, 1H), 1.54-1.35 (m, 3H), 0.59 (d, J=6.4 Hz, 2H), 0.45-0.40 (m, 2H).

### Example 28 Synthesis report of 1-(5-(5-((3R,5R)-3-amino-5-fluoropiperidine-1-carbonyl)-7-methoxy-1-methyl-1H-benzo[d]imidazol-2-yl)-2,3-dihydro-1H-pyrrolo[1,2,3-de]quinoxalin-1-yl)-2-hydroxyethan-1-one

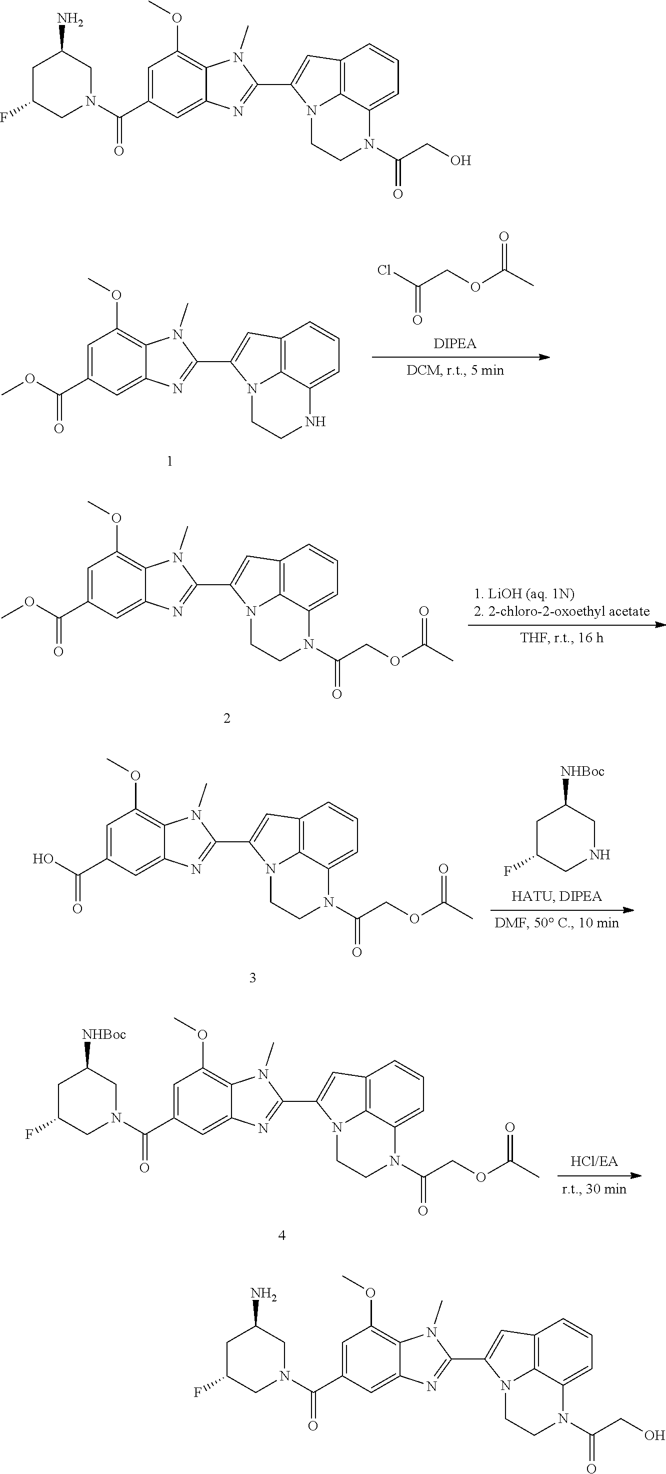

### Step 1:

Methyl 2-(1,9-diazatricyclo[6.3.1.04,12]dodeca-2,4(12), 5,7-tetraen-2-yl)-7-methoxy-1-methyl-benzimidazole-5-carboxylate (50 mg, 132.83 μmol) and DIPEA (51.5 mg, 398.50 μmol, 69.41 μL) were dissolved in dichloromethane (5 mL). The mixture was stirred at 0° C. and (2-chloro-2-oxo-ethyl) acetate (18.1 mg, 132.83 μmol) was added into the mixture dropwise at 0° C. The reaction mixture was stirred at RT for 5 min until desired signal was found by LC/MS. After removal of the solvent in vacuo, the residue was purified by flash column chromatography on silica gel (eluting with EA/PE from 0 to 80%) to give methyl 2-[9-(2-acetoxyacetyl)-1,9-diazatricyclo[6.3.1.04,12]dodeca-2,4(12), 5,7-tetraen-2-yl]-7-methoxy-1-methyl-benzimidazole-5-carboxylate (50 mg, 104.94 μmol, 79.00% yield) as yellow oil. LC/MS (ESI⁺) [(M+H)⁺]: 476.7.

### Step 2:

Methyl 2-[9-(2-acetoxyacetyl)-1,9-diazatricyclo[6.3.1.04,12]dodeca-2,4(12), 5,7-tetraen-2-yl]-7-methoxy-1-methyl-benzimidazole-5-carboxylate (50 mg, 104.94 μmol) was dissolved in THF (1 mL). LiOH (aq. 1 N) (105.14 μmol, 2 mL) was added into the solution. The reaction nmixture was stirred at RT for overnight. After removal of the solvent and the residue was dissolved in THF (5 mL). The mixture was stirred at 0° C. and 2-chloro-2-oxoethyl acetate (14.33 mg, 104.94 μmol) was added into the mixture dropwise at 0° C. After removal of the solvent in vacuo, the residue was purified by flash column chromatography on silica gel (eluting with EA/PE from 0 to 80%) and pre-HPLC to give 2-[9-(2-acetoxyacetyl)-1,9-diazatricyclo[6.3.1.04,12]dodeca-2,4(12), 5,7-tetraen-2-yl]-7-methoxy-1-methyl-benzimidazole-5-carboxylic acid (30 mg, 64.87 μmol, 61.82% yield) as a yellow solid. LC/MS(ESI⁺) [(M+H)⁺]: 462.7.

### Step 3:

A mixture of 2-[9-(2-acetoxyacetyl)-1,9-diazatricyclo[6.3.1.04,12]dodeca-2,4(12), 5,7-tetraen-2-yl]-7-methoxy-1-methyl-benzimidazole-5-carboxylic acid (30 mg, 64.87 μmol), tert-butyl N-[(3R,5R)-5-fluoro-3-piperidyl]carbamate (14.2 mg, 64.87 μmol), HATU (24.7 mg, 64.87 μmol) and DIPEA (25.2 mg, 194.61 μmol, 33.90 μL) was dissolved in DMF (3 mL). The reaction mixture was stirred at 50° C. for 10 min. The reaction mixture was diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give [2-[2-[5-[(3R,5R)-3-(tert-butoxycarbonylamino)-5-fluoro-piperidine-1-carbonyl]-7-methoxy-1-methyl-benzimidazol-2-yl]-1,9-diazatricyclo[6.3.1.04,12]dodeca-2,4(12), 5,7-tetraen-9-yl]-2-oxo-ethyl]acetate (20 mg, 30.18 μmol, 46.52% yield) as yellow oil. LC/MS(ESI⁺) [(M+H)⁺]: 662.7.

### Step 4:

[2-[2-[5-[(3R,5R)-3-(tert-butoxycarbonylamino)-5-fluoro-piperidine-1-carbonyl]-7-methoxy-1-methyl-benzimidazol-2-yl]-1,9-diazatricyclo[6.3.1.04,12]dodeca-2,4(12), 5,7-tetraen-9-yl]-2-oxo-ethyl]acetate (15 mg, 22.63 μmol) was dissolved in THF (1 mL). LiOH (aq. 1 N) (38.64 μmol, 2 mL) was added into the solution. The reaction mixture was stirred at rt for 10 min. Then removed the solvent in vacuo and HCl/EA (2 mL) was added into the mixture. The mixture was stirred at rt for 10 min, concentrated in vacuo. The residue was purified by pre-HPLC to give 1-[2-[5-[(3R,5R)-3-amino-5-fluoro-piperidine-1-carbonyl]-7-methoxy-1-methyl-benzimidazol-2-yl]-1,9-diazatricyclo[6.3.1.04,12]dodeca-2,4(12), 5,7-tetraen-9-yl]-2-hydroxy-ethanone (5 mg, 9.61 μmol, 42.44% yield) as a white solid. LC/MS(ESI⁺) [(M+H)⁺]: 520.8. ¹H NMR (400 MHz, DMSO-d₆) δ 8.23 (s, 1H), 7.48 (d, J=8.0 Hz, 1H), 7.32 (s, 1H), 7.16 (s, 1H), 7.12 (t, J=7.8 Hz, 1H), 6.86 (s, 1H), 5.01 (s, 1H), 4.66 (s, 2H), 4.44 (d, J=3.8 Hz, 2H), 4.25 (s, 3H), 4.13 (s, 2H), 4.00 (s, 3H), 3.02 (s, 2H), 2.68 (s, 1H), 2.17 (s, 2H), 1.61-1.49 (m, 2H).

### The Following Compounds were Prepared Analogously

### Example 29 Synthesis report of 2-(5-(5-((3R,5R)-3-amino-5-fluoropiperidine-1-carbonyl)-7-methoxy-1-methyl-1H-benzo[d]imidazol-2-yl)-2,3-dihydro-1H-pyrrolo[1,2,3-de]quinoxalin-1-yl)-2-oxoethyl acetate)

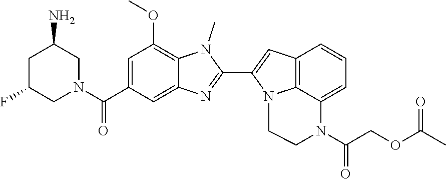 [SMILES: COc1cc(C(=O)N2C[C@H](N)C[C@@H](F)C2)cc2nc(-c3cc4cccc5c4n3CCN5C(=O)COC(C)=O)n(C)c12]

Prepared in analogous manner as for Example 28. LC/MS(ESI⁺) [(M+H)⁺]: 562.7.

### Example 30 Synthesis report of 3-(5-(5-((3R,5R)-3-amino-5-fluoropiperidine-1-carbonyl)-7-methoxy-1-methyl-1H-benzo[d]imidazol-2-yl)-2,3-dihydro-1H-pyrrolo[1,2,3-de]quinoxalin-1-yl)-3-oxopropanenitrile

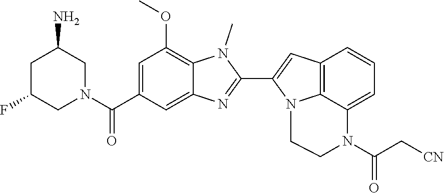 [SMILES: COc1cc(C(=O)N2C[C@H](N)C[C@@H](F)C2)cc2nc(-c3cc4cccc5c4n3CCN5C(=O)CC#N)n(C)c12]

Prepared in analogous manner as for Example 28. LC/MS(ESI⁺) [(M+H)⁺]: 529.8.

### Example 31 Synthesis of (R)-(3-aminopiperidin-1-yl)(2-(1-benzoyl-2,3-dihydro-1H-pyrrolo[1,2,3-de]quinoxalin-5-yl)-7-methoxy-1-methyl-1H-benzo[d]imidazol-5-yl)methanone

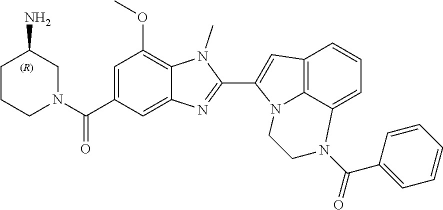

Prepared in analogous manner as for Example 28. LC/MS (ESI⁺) [(M+H)⁺]: 549.

### Example 32 Preparation of (R)-(2-(1-(3-aminocyclobutyl)-2,3-dihydro-1H-pyrrolo[1,2,3-de]quinoxalin-5-yl)-7-methoxy-1-methyl-1H-benzo[d]imidazol-5-yl)(3-aminopiperidin-1-yl)methanone

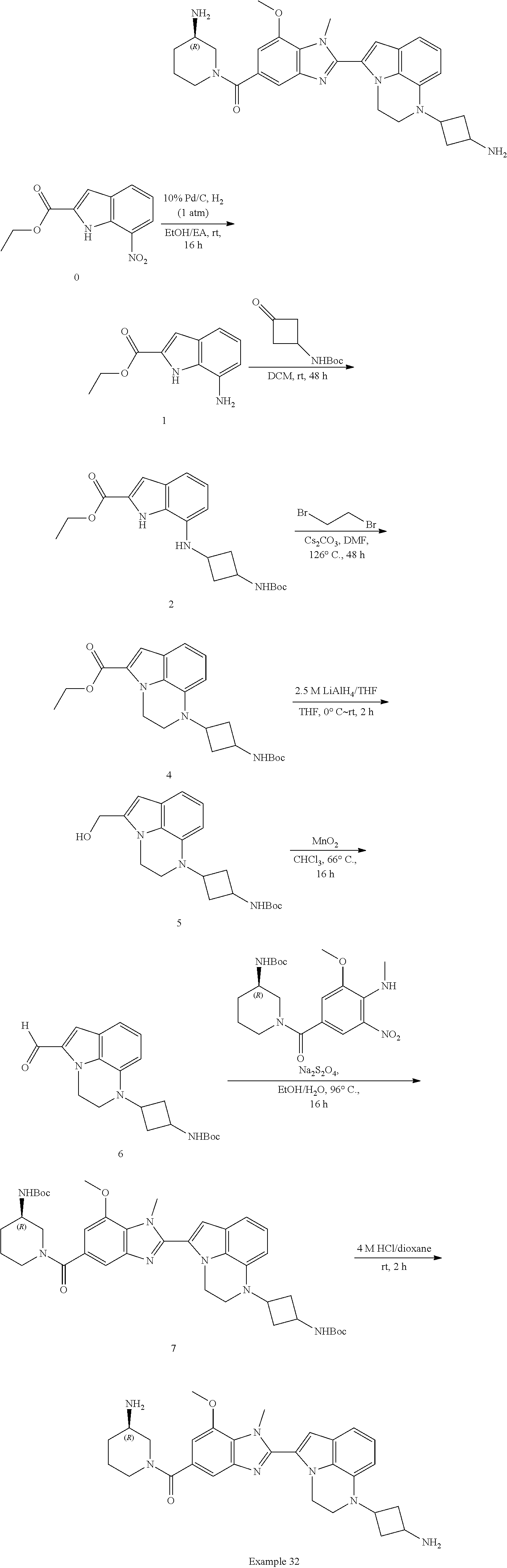

### Step 1:

To a solution of ethyl 7-nitro-1H-indole-2-carboxylate (2 g, 8.54 mmol) in ethanol (50 mL) and ethyl acetate, 99% (50 mL) was added palladium (200 mg, 1.88 mmol) at RT. The reaction mixture was purged with H₂, stirred at RT 16 h and filtered. The filtrate was concentrated in vacuo, and the residue was triturated in EA/PE (1/10, 110 mL). The light brown solid was collected by filtration and dried in vacuo to afford ethyl 7-amino-1H-indole-2-carboxylate (1.7 g, 8.32 mmol, 97.48% yield). LC/MS (ESI⁺) [(M+H)⁺]: 205.

### Step 2:

To a solution of ethyl 7-amino-1H-indole-2-carboxylate (1 g, 4.90 mmol) and tert-butyl N-(3-oxocyclobutyl)carbamate (1.09 g, 5.88 mmol) in DCM (100 mL) was added sodium triacetoxyboranuide (5.19 g, 24.48 mmol) in portions at RT. The resulting reaction mixture was stirred at RT for 48 h and quenched with ice (100 mL). The two phases were separated, and the aqueous phase was extracted with DCM (60 mL*3). The combined organic phase was dried over anhydrous sodium sulfate and filtered. The filtrate was concentrated in vacuo, and the residue was purified by flash column chromatography on silica gel (PE/EA: 3/1, Rf=0.5) to provide ethyl 7-[[3-(tert-butoxycarbonylamino)cyclobutyl]amino]-1H-indole-2-carboxylate (1.65 g, 4.42 mmol, 90.23% yield) as a white solid. LC/MS (ESI⁺) [(M+H)⁺]: 374.

### Step 3:

To a solution of ethyl 7-[[3-(tert-butoxycarbonylamino)cyclobutyl]amino]-1H-indole-2-carboxylate (1.5 g, 4.02 mmol) in anhydrous DMF (40 mL) were added dicesium carbonate (3.93 g, 12.05 mmol) and 1,2-dibromoethane (830.03 mg, 4.42 mmol, 380.75 μL) at rt. The reaction mixture was stirred at 126° C. for 48 h (the conversion was only 25%), cooled to RT and filtered. The filtrate was diluted with EA (300 mL), washed with water (50 mL*5) and brine (100 mL), dried over anhydrous sodium sulfate and filtered. The filtrate was concentrated in vacuo, and the residue was purified by flash column chromatography on silica gel (PE/EA: 3/1, Rf=0.55) to provide ethyl 9-[3-(tert-butoxycarbonylamino)cyclobutyl]-1,9-diazatricyclo[6.3.1.0^{4,12}]dodeca-2,4,6,8(12)-tetraene-2-carboxylate (100 mg, 250.32 μmol, 6.23% yield), LC/MS (ESI⁺) [(M+H)⁺]: 400.

### Step 4:

To a solution of ethyl 9-[3-(tert-butoxycarbonylamino)cyclobutyl]-1,9-diazatricyclo[6.3.1.0^{4,12}]dodeca-2,4,6,8(12)-tetraene-2-carboxylate (40 mg, 100.13 μmol) in anhydrous THF (5 mL) was added aluminum; lithium (2.5 M, 160.21 μL) dropwise at 0° C. The reaction mixture was stirred at RT for 2 h, cooled to 0° C., quenched with EA (10 mL) and stirred for 30 min. The mixture was filtered, and the filtrate was concentrated in vacuo to afford tert-butyl N-[3-[2-(hydroxymethyl)-1,9-diazatricyclo[6.3.1.0^{4,12}]dodeca-2,4,6,8(12)-tetraen-9-yl]cyclobutyl]carbamate (35 mg, 97.92 μmol, 97.79% yield) as a light-yellow solid. LC/MS (ESI⁺) [(M+H)⁺]: 358.

### Step 5:

To s solution of tert-butyl N-[3-[2-(hydroxymethyl)-1,9-diazatricyclo[6.3.1.0^{4,12}]dodeca-2,4,6,8(12)-tetraen-9-yl]cyclobutyl]carbamate (35 mg, 97.92 μmol) in chloroform, 99.8%, ACS Reagent (10 mL) was added manganyl oxygen(2-) (85.1 mg, 979.17 μmol). The reaction mixture was stirred at 60° C. for 16 h, cooled to RT and filtered through a pad of Celite. The solid cake was washed with DCM (20 mL), and the filtrate was concentrated in vacuo to afford tert-butyl N-[3-(2-formyl-1,9-diazatricyclo[6.3.1.0^{4,12}]dodeca-2,4,6,8(12)-tetraen-9-yl)cyclobutyl]carbamate (33 mg, 92.85 μmol) as a yellow oil. LC/MS (ESI⁺) [(M+H)⁺]: 356.

### Step 6:

To a solution of tert-butyl N-[3-(2-formyl-1,9-diazatricyclo[6.3.1.0^{4,12}]dodeca-2,4,6,8(12)-tetraen-9-yl)cyclobutyl]carbamate (15 mg, 42.20 μmol) and tert-butyl N-[(3R)-1-[3-methoxy-4-(methylamino)-5-nitro-benzoyl]-3-piperidyl]carbamate (20 mg, 48.97 μmol) in ethanol (3 mL) were added Na₂S₂O₄ (29.4 mg, 168.81 μmol) and water (3 mL). The reaction mixture was stirred at 96° C. 16 h and concentrated in vacuo. The residue was extracted with DCM (10 mL*3), and the combined organic phase was dried over anhydrous sodium sulfate and filtered. The filtrate was concentrated in vacuo, and the residue was purified by prep-TLC (DCM/MeOH: 15/1, Rf=0.4) to provide tert-butyl N-[(3R)-1-[2-[9-[3-(tert-butoxycarbonylamino)cyclobutyl]-1,9-diazatricyclo[6.3.1.0^{4,12}]dodeca-2,4(12), 5,7-tetraen-2-yl]-7-methoxy-1-methyl-benzimidazole-5-carbonyl]-3-piperidyl]carbamate (10 mg, 14.01 μmol, 33.19% yield) was a yellow solid. LC/MS (ESI⁺) [(M+H)⁺]: 714.

### Step 7:

A mixture of tert-butyl N-[(3R)-1-[2-[9-[3-(tert-butoxycarbonylamino)cyclobutyl]-1,9-diazatricyclo[6.3.1.0^{4,12}]dodeca-2,4(12), 5,7-tetraen-2-yl]-7-methoxy-1-methyl-benzimidazole-5-carbonyl]-3-piperidyl]carbamate (10 mg, 14.01 μmol) in Hydrogen chloride solution 4.0 M in dioxane (2 mL) was stirred at RT for 2 h and concentrated in vacuo, and the residue was purified by prep-HPLC to provide [2-[9-(3-aminocyclobutyl)-1,9-diazatricyclo[6.3.1.0^{4,12}]dodeca-2,4(12), 5,7-tetraen-2-yl]-7-methoxy-1-methyl-benzimidazol-5-yl]-[(3R)-3-amino-1-piperidyl]methanone (1 mg, 1.95 μmol) as a yellow solid, LC/MS (ESI⁺) [(M+H)⁺]: 514.

### Example 33 Preparation of (R)—N-(3-(5-(5-(3-aminopiperidine-1-carbonyl)-7-methoxy-1-methyl-1H-benzo[d]imidazol-2-yl)-2,3-dihydro-1H-pyrrolo[1,2,3-de]quinoxalin-1-yl)cyclobutyl)acetamide

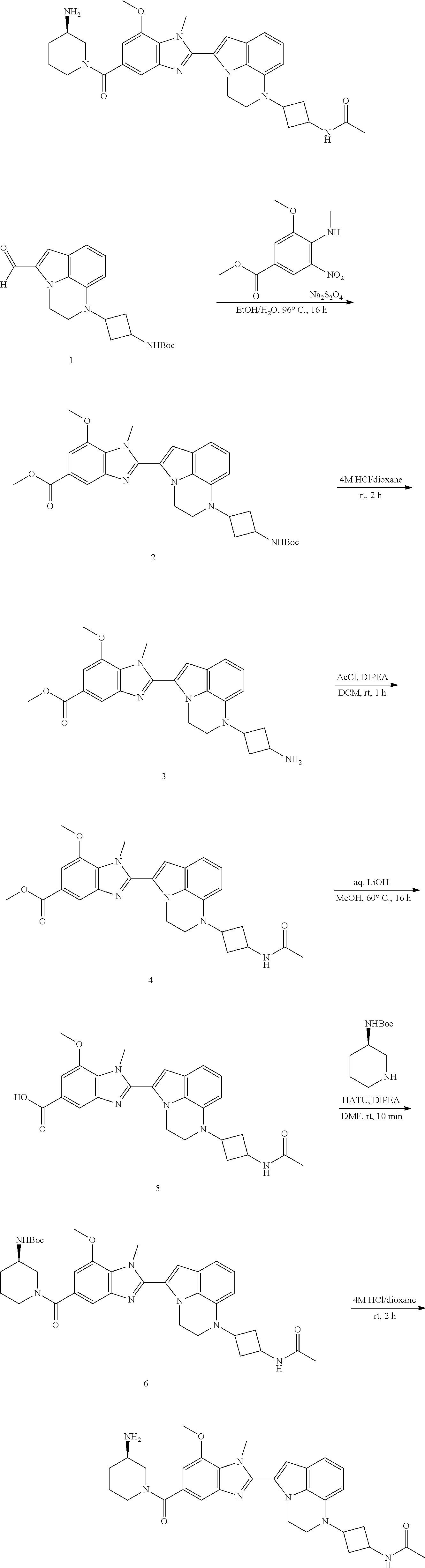

### Step 1:

To a solution of tert-butyl N-[3-(2-formyl-1,9-diazatricyclo[6.3.1.0^{4,12}]dodeca-2,4,6,8(12)-tetraen-9-yl)cyclobutyl]carbamate (15 mg, 42.20 μmol) and methyl 3-methoxy-4-(methylamino)-5-nitro-benzoate (15 mg, 62.44 μmol) were added Na₂S₂O₄ (29.4 mg, 168.81 μmol) and water (3 mL). The reaction mixture was stirred at 96° C. for 16 h and concentrated in vacuo. The residue was extracted with DCM (10 mL*3), and the combined organic phase was dried over anhydrous sodium sulfate and filtered. The filtrate was concentrated in vacuo, and the residue was purified by prep-TLC (PE/EA: 3/1, Rf=0.4) to provide methyl 2-[9-[3-(tert-butoxycarbonylamino)cyclobutyl]-1,9-diazatricyclo[6.3.1.0^{4,12}]dodeca-2,4(12), 5,7-tetraen-2-yl]-7-methoxy-1-methyl-benzimidazole-5-carboxylate (10 mg, 18.33 μmol, 43.43% yield) was a yellow solid, LC/MS (ESI⁺) [(M+H)⁺]: 546.

### Step 2:

To a solution of methyl 2-[9-[3-(tert-butoxycarbonylamino)cyclobutyl]-1,9-diazatricyclo[6.3.1.0^{4,12}]dodeca-2,4(12), 5,7-tetraen-2-yl]-7-methoxy-1-methyl-benzimidazole-5-carboxylate (10 mg, 18.33 μmol) in dioxane (2 mL) was added hydrogen chloride solution 4.0M was stirred at RT for 2 h and concentrated in vacuo to provide crude methyl 2-[9-(3-aminocyclobutyl)-1,9-diazatricyclo[6.3.1.0^{4,12}]dodeca-2,4(12), 5,7-tetraen-2-yl]-7-methoxy-1-methyl-benzimidazole-5-carboxylate (8 mg, 17.96 μmol) as a yellow solid, LC/MS (ESI⁺) [(M+H)⁺]: 446. The crude product was used in the next step without further purifications.

### Step 3:

To a solution of methyl 2-[9-(3-aminocyclobutyl)-1,9-diazatricyclo[6.3.1.0^{4,12}]dodeca-2,4(12), 5,7-tetraen-2-yl]-7-methoxy-1-methyl-benzimidazole-5-carboxylate (8 mg, 17.96 μmol) in DCM (2 mL) were added N-ethyl-N-isopropyl-propan-2-amine (74.2 mg, 574.13 μmol, 0.1 mL) and acetyl chloride (10 mg, 127.39 μmol, 7.75 μL) at rt. The reaction mixture was stirred at rt for 1 h and diluted with DCM (20 mL) and water (10 mL). The two phases were separated, and the aqueous phase was extracted with DCM (10 mL*3). The combined organic phase was dried over anhydrous sodium sulfate and filtered. The filtrate was concentrated in vacuo to provide methyl 2-[9-(3-acetamidocyclobutyl)-1,9-diazatricyclo[6.3.1.0^{4,12}]dodeca-2,4(12), 5,7-tetraen-2-yl]-7-methoxy-1-methyl-benzimidazole-5-carboxylate (8 mg, 16.41 μmol) as a yellow solid, LC/MS (ESI⁺) [(M+H)⁺]: 488, which was used in the next step without further purification.

### Step 4:

A mixture of methyl 2-[9-(3-acetamidocyclobutyl)-1,9-diazatricyclo[6.3.1.0^{4,12}]dodeca-2,4(12), 5,7-tetraen-2-yl]-7-methoxy-1-methyl-benzimidazole-5-carboxylate (8 mg, 16.41 μmol) and lithium hydroxide hydrate (3.4 mg, 82.04 μmol, 2.28 μL) in mixed solvent of methanol (3 mL) and water (0.3 mL) was stirred at 60° C. for 16 h and concentrated in vacuo. The residue was acidified to pH ˜3 with 2N HCl aqueous solution and extracted with DCM (10 mL*3). The product remained in aqueous phase, which was lyophilized to provide a crude product and used for the next step without purification.

### Step 5:

To a solution of 2-[9-(3-acetamidocyclobutyl)-1,9-diazatricyclo[6.3.1.0^{4,12}]dodeca-2,4(12), 5,7-tetraen-2-yl]-7-methoxy-1-methyl-benzimidazole-5-carboxylic acid (5 mg, 10.56 μmol), tert-butyl N-[(3R)-3-piperidyl]carbamate (2.1 mg, 10.56 μmol) and [dimethylamino(triazolo[4,5-b]pyridin-3-yloxy)methylene]-dimethyl-ammonium; hexafluorophosphate (4.0 mg, 10.56 μmol) in DMF (2 mL) was added N-ethyl-N-isopropyl-propan-2-amine (6.8 mg, 52.80 μmol, 9.20 μL) at RT. The reaction mixture was stirred at RT for 10 min and purified by prep-HPLC to provide tert-butyl N-[(3R)-1-[2-[9-(3-acetamidocyclobutyl)-1,9-diazatricyclo[6.3.1.0^{4,12}]dodeca-2,4(12), 5,7-tetraen-2-yl]-7-methoxy-1-methyl-benzimidazole-5-carbonyl]-3-piperidyl]carbamate (3 mg, 4.57 μmol) as a yellow solid, LC/MS (ESI⁺) [(M+H)⁺]: 656.

### Step 6:

A mixture of tert-butyl N-[(3R)-1-[2-[9-(3-acetamidocyclobutyl)-1,9-diazatricyclo[6.3.1.0^{4,12}]dodeca-2,4(12), 5,7-tetraen-2-yl]-7-methoxy-1-methyl-benzimidazole-5-carbonyl]-3-piperidyl]carbamate (3 mg, 4.57 μmol) in hydrogen chloride solution 4.0 M in dioxane (2 mL) was stirred at RT for 2 h and concentrated in vacuo to provide N-[3-[2-[5-[(3R)-3-aminopiperidine-1-carbonyl]-7-methoxy-1-methyl-benzimidazol-2-yl]-1,9-diazatricyclo[6.3.1.0^{4,12}]dodeca-2,4(12), 5,7-tetraen-9-yl]cyclobutyl]acetamide (1 mg, 1.80 μmol, 39.34% yield) as a yellow solid. LC/MS (ESI⁺) [(M+H)⁺]: 556.

### Example 34 Synthesis report of ((3R,5R)-3-amino-5-fluoropiperidin-1-yl)(1-((1-ethyl-1H-pyrazol-4-yl)methyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

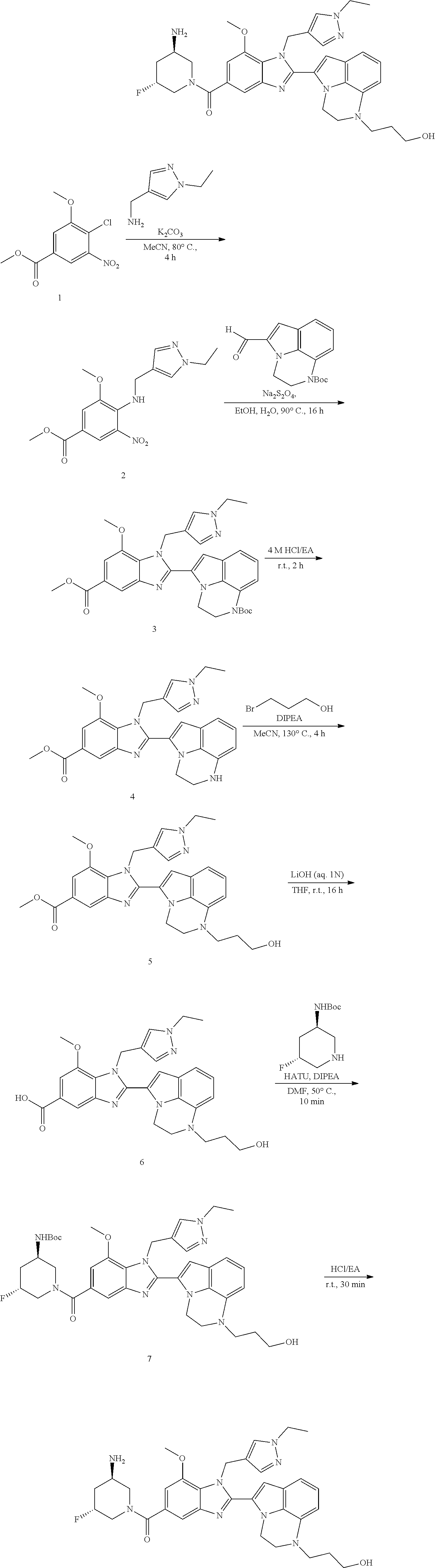

### Step 1:

A mixture of methyl 4-chloro-3-methoxy-5-nitro-benzoate (245 mg, 997.49 μmol), (1-ethylpyrazol-4-yl)methenamine (137.3 mg, 1.10 mmol) and potassium carbonate (413.6 mg, 2.99 mmol, 180.61 μL) was dissolved in acetonitrile (10 mL). The mixture was stirred at 80° C. for 4 h. After cooling to RT, the solvent was removed in vacuo. The residue was purified by flash column chromatography on silica gel (eluting with EA/PE from 0 to 50%) to give methyl 4-[(1-ethylpyrazol-4-yl) methylamino]-3-methoxy-5-nitro-benzoate (300 mg, 897.33 μmol, 89.96% yield) as a yellow solid. LC/MS(ESI⁺) [(M+H)⁺]: 334.8.

### Step 2:

A mixture of methyl 4-[(1-ethylpyrazol-4-yl)methylamino]-3-methoxy-5-nitro-benzoate (300 mg, 897.33 μmol), tert-butyl 2-formyl-1,9-diazatricyclo[6.3.1.04,12]dodeca-2,4(12), 5,7-tetraene-9-carboxylate (256.9 mg, 897.33 μmol) and sodium hydrosulfite (468.7 mg, 2.69 mmol) was dissolved in ethanol/water (30 mL). The reaction mixture was stirred at 100° C. for overnight. After removal of the solvent in vacuo, the residue was purified by flash column chromatography on silica gel (eluting with EA/PE from 0 to 100%) to give tert-butyl 2-[1-[(1-ethylpyrazol-4-yl)methyl]-7-methoxy-5-methoxycarbonyl-benzimidazol-2-yl]-1,9-diazatricyclo[6.3.1.04,12]dodeca-2,4(12), 5,7-tetraene-9-carboxylate (300 mg, 525.73 μmol, 78.12% yield) as liquid oil. LC/MS(ESI⁺) [(M+H)⁺]: 570.8.

### Step 3:

Tert-butyl 2-[1-[(1-ethylpyrazol-4-yl)methyl]-7-methoxy-5-methoxycarbonyl-benzimidazol-2-yl]-1,9-diazatricyclo[6.3.1.04,12]dodeca-2,4(12), 5,7-tetraene-9-carboxylate (300 mg, 525.73 μmol) was dissolved in HCl/EA (10 mL). The reaction mixture was stirred at RT for 2 h. Some solid appeared. The mixture was filtered and the filtrate was dried in vacuo to give methyl 2-(1,9-diazatricyclo[6.3.1.04,12]dodeca-2,4(12), 5,7-tetraen-2-yl)-1-[(1-ethylpyrazol-4-yl)methyl]-7-methoxy-benzimidazole-5-carboxylate (200 mg, 425.06 μmol, 80.85% yield) as a gray solid. LC/MS(ESI⁺) [(M+H)⁺]: 470.8.

### Step 4:

A mixture of methyl 2-(1,9-diazatricyclo[6.3.1.04,12]dodeca-2,4(12), 5,7-tetraen-2-yl)-1-[(1-ethylpyrazol-4-yl)methyl]-7-methoxy-benzimidazole-5-carboxylate (100 mg, 212.53 μmol), 3-bromopropan-1-ol (59.1 mg, 425 μmol, 37.15 μL) and DIPEA (27.5 mg, 37.02 uL) was dissolved in Acetonitrile (5 mL). The reaction mixture was stirred at 130° C. for 4 h in microwave reactor. After removal of the solvent in vacuo, the residue was purified by flash column chromatography on silica gel (eluting with EA/PE from 0 to 100) to give methyl 1-[(1-ethylpyrazol-4-yl)methyl]-2-[9-(3-hydroxypropyl)-1,9-diazatricyclo[6.3.1.04,12]dodeca-2,4(12), 5,7-tetraen-2-yl]-7-methoxy-benzimidazole-5-carboxylate (30 mg, 56.75 μmol) as liquid oil. LC/MS(ESI⁺) [(M+H)⁺]: 528.8.

### Step 5:

To a solution of methyl 1-[(1-ethylpyrazol-4-yl)methyl]-2-[9-(3-hydroxypropyl)-1,9-diazatricyclo[6.3.1.04,12]dodeca-2,4(12), 5,7-tetraen-2-yl]-7-methoxy-benzimidazole-5-carboxylate (30 mg, 56.75 μmol) in THF (1 mL) was added LiOH (aq. 1 N) (19.36 μmol, 2 mL). The resulting mixture was stirred at RT for overnight. The pH of reaction mixture was adjusted to be acidic with 2 mol/L HCl. After removal of the solvent in vacuo, the crude product 1-[(1-ethylpyrazol-4-yl)methyl]-2-[9-(3-hydroxypropyl)-1,9-diazatricyclo[6.3.1.04,12]dodeca-2,4(12), 5,7-tetraen-2-yl]-7-methoxy-benzimidazole-5-carboxylic acid (25 mg, 48.58 μmol, 85.6% yield) was obtained as a yellow solid. The crude product was used in next reaction without further purification. LC/MS(ESI⁺) [(M+H)⁺]: 514.8.

### Step 6

A mixture of 1-[(1-ethylpyrazol-4-yl)methyl]-2-[9-(3-hydroxypropyl)-1,9-diazatricyclo[6.3.1.04,12]dodeca-2,4(12), 5,7-tetraen-2-yl]-7-methoxy-benzimidazole-5-carboxylic acid (25 mg, 48.58 μmol), tert-butyl N-[(3R,5R)-5-fluoro-3-piperidyl]carbamate (10.6 mg, 48.58 μmol), HATU (18.5 mg, 48.58 μmol) and DIPEA (18.9 mg, 146 μmol, 25.43 μL) was dissolved in DMF (5 mL). The mixture was stirred at 50° C. for 10 min. Desired signal was found by LC/MS. The reaction was diluted with EtOAc (50 ml) and washed with water (25 ml). The organic layer was dried over sodium sulfate, filtered, and concentrated in vacuo. The crude product was purified by flash column chromatography on silica gel (eluting with EA/PE from 0 to 100%) to give tert-butyl N-[(3R,5R)-1-[1-[(1-ethylpyrazol-4-yl)methyl]-2-[9-(3-hydroxypropyl)-1,9-diazatricyclo[6.3.1.04,12]dodeca-2,4(12), 5,7-tetraen-2-yl]-7-methoxy-benzimidazole-5-carbonyl]-5-fluoro-3-piperidyl]carbamate (20 mg, 27.98 μmol, 57.59% yield) as yellow oil. LC/MS(ESI⁺) [(M+H)⁺]: 714.7.

### Step 7:

Tert-butyl N-[(3R,5R)-1-[1-[(1-ethylpyrazol-4-yl)methyl]-2-[9-(3-hydroxypropyl)-1,9-diazatricyclo[6.3.1.04,12]dodeca-2,4(12), 5,7-tetraen-2-yl]-7-methoxy-benzimidazole-5-carbonyl]-5-fluoro-3-piperidyl]carbamate (20 mg, 27.98 μmol) was dissolved in HCl/EA (2 mL). The mixture was stirred at RT for 30 min. After removal of the solvent in vacuo and purified by pre-HPLC to afford [(3R,5R)-3-amino-5-fluoro-1-piperidyl]-[1-[(1-ethylpyrazol-4-yl)methyl]-2-[9-(3-hydroxypropyl)-1,9-diazatricyclo[6.3.1.04,12]dodeca-2,4(12), 5,7-tetraen-2-yl]-7-methoxy-benzimidazol-5-yl]methanone (10 mg, 16.27 μmol, 58.14% yield) as white solid. LC/MS(ESI⁺) [(M+H)⁺]: 614.8.

### The Following Compounds were Prepared Analogously

### Example 35 Synthesis of ((3R,5R)-3-amino-5-fluoropiperidin-1-yl)(2-(1-(3-hydroxy-3-methylbutyl)-2,3-dihydro-1H-pyrrolo[1,2,3-de]quinoxalin-5-yl)-7-methoxy-1-((1-methyl-1H-pyrazol-4-yl)methyl)-1H-benzo[d]imidazol-5-yl)methanone

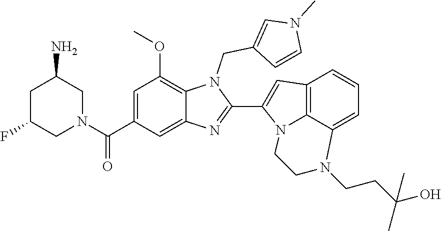 [SMILES: COc1cc(C(=O)N2C[C@H](N)C[C@@H](F)C2)cc2nc(-c3cc4cccc5c4n3CCN5CCC(C)(C)O)n(Cc3ccn(C)c3)c12]

Prepared in analogous manner as for Example 34. LC/MS(ESI⁺) [(M+H)⁺]: 628.8. ¹H NMR (400 MHz, DMSO) δ 8.27 (s, 1H), 7.46 (s, 1H), 7.36 (s, 1H), 7.14 (s, 1H), 7.02-6.98 (m, 2H), 6.97 (s, 1H), 6.91 (s, 1H), 6.46 (d, J=5.4 Hz, 1H), 5.74 (s, 2H), 4.54 (s, 2H), 4.41 (s, 1H), 4.02 (s, 3H), 3.78 (s, 3H), 3.56 (d, J=6.0 Hz, 5H), 2.73 (s, 1H), 2.39 (s, 1H), 2.06 (d, J=7.6 Hz, 2H), 1.81 (d, J=8.2 Hz, 2H), 1.30 (s, 3H), 1.25 (s, 3H).

### Example 36 Synthesis report of ((3R,5R)-3-amino-5-fluoropiperidin-1-yl)(1-(4-fluorobenzyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

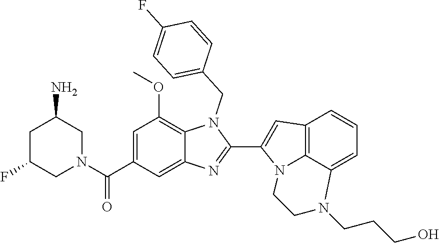 [SMILES: COc1cc(C(=O)N2C[C@H](N)C[C@@H](F)C2)cc2nc(-c3cc4cccc5c4n3CCN5CCCO)n(Cc3ccc(F)cc3)c12]

Prepared in analogous manner as for Example 34. LC/MS(ESI⁺) [(M+H)⁺]: 614.8.

### Example 37 Synthesis report of ((3R,5R)-3-amino-5-fluoropiperidin-1-yl)(2-(1-(3-hydroxypropyl)-2,3-dihydro-1H-pyrrolo[1,2,3-de]quinoxalin-5-yl)-7-methoxy-1-(thiophen-2-ylmethyl)-1H-benzo[d]imidazol-5-yl)methanone

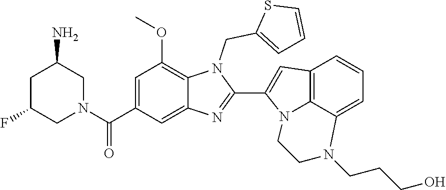 [SMILES: COc1cc(C(=O)N2C[C@H](N)C[C@@H](F)C2)cc2nc(-c3cc4cccc5c4n3CCN5CCCO)n(Cc3cccs3)c12]

Prepared in analogous manner as for Example 34. LC/MS(ESI⁺) [(M+H)⁺]: 602.7.

### Example 38 Synthesis report of ((3R,5R)-3-amino-5-fluoropiperidin-1-yl)(1-((1-ethyl-1H-pyrazol-4-yl)methyl)-7-fluoro-2-(1-(3-hydroxypropyl)-2,3-dihydro-1H-pyrrolo[1,2,3-de]quinoxalin-5-yl)-1H-benzo[d]imidazol-5-yl)methanone

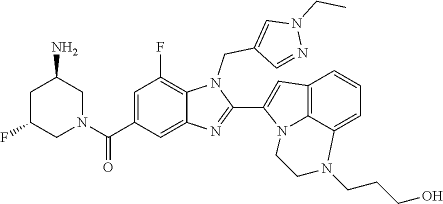 [SMILES: CCn1cc(Cn2c(-c3cc4cccc5c4n3CCN5CCCO)nc3cc(C(=O)N4C[C@H](N)C[C@@H](F)C4)cc(F)c32)cn1]

Prepared in analogous manner as for Example 34. LC/MS(ESI⁺) [(M+H)⁺]: 602.8.

### Example 39 Synthesis report of ((3R,5R)-3-amino-5-fluoropiperidin-1-yl)(1-((1-ethyl-1H-pyrazol-4-yl)methyl)-7-fluoro-2-(1-(2-hydroxyethyl)-2,3-dihydro-1H-pyrrolo[1,2,3-de]quinoxalin-5-yl)-1H-benzo[d]imidazol-5-yl)methanone

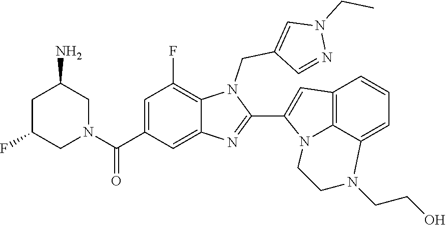 [SMILES: CCn1cc(Cn2c(-c3cc4cccc5c4n3CCN5CCO)nc3cc(C(=O)N4C[C@H](N)C[C@@H](F)C4)cc(F)c32)cn1]

Prepared in analogous manner as for Example 34. LC/MS(ESI⁺) [(M+H)⁺]: 588.8.

### Example 40 Synthesis report of ((3R,5R)-3-amino-5-fluoropiperidin-1-yl)(2-(2,3-dihydro-1H-pyrrolo[1,2,3-de]quinoxalin-5-yl)-1-((1-ethyl-1H-pyrazol-4-yl)methyl)-7-fluoro-1H-benzo[d]imidazol-5-yl)methanone

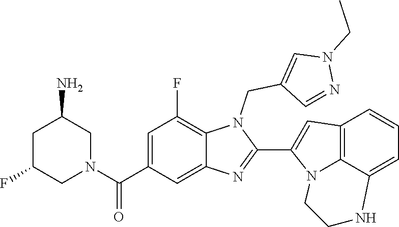 [SMILES: CCn1cc(Cn2c(-c3cc4cccc5c4n3CCN5)nc3cc(C(=O)N4C[C@H](N)C[C@@H](F)C4)cc(F)c32)cn1]

Prepared in analogous manner as for Example 34. LC/MS(ESI⁺) [(M+H)⁺]: 544.8.

### Example 41 Synthesis report of ((3R,5R)-3-amino-5-fluoropiperidin-1-yl)(1-((1-ethyl-1H-pyrazol-4-yl)methyl)-7-fluoro-2-(1-(3-methoxypropyl)-2,3-dihydro-1H-pyrrolo[1,2,3-de]quinoxalin-5-yl)-1H-benzo[d]imidazol-5-yl)methanone

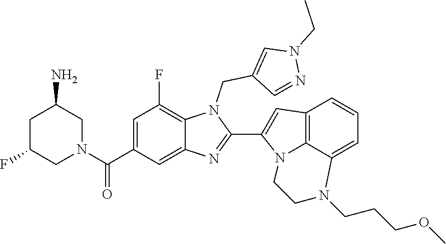 [SMILES: CCn1cc(Cn2c(-c3cc4cccc5c4n3CCN5CCCOC)nc3cc(C(=O)N4C[C@H](N)C[C@@H](F)C4)cc(F)c32)cn1]

Prepared in analogous manner as for Example 34. LC/MS(ESI⁺) [(M+H)⁺]: 616.8.

### Example 42 Synthesis report of ((1R,4R,7R)-7-amino-2-azabicyclo[2.2.1]heptan-2-yl)(1-((1-ethyl-1H-pyrazol-4-yl)methyl)-7-fluoro-2-(1-(3-hydroxypropyl)-2,3-dihydro-1H-pyrrolo[1,2,3-de]quinoxalin-5-yl)-1H-benzo[d]imidazol-5-yl)methanone

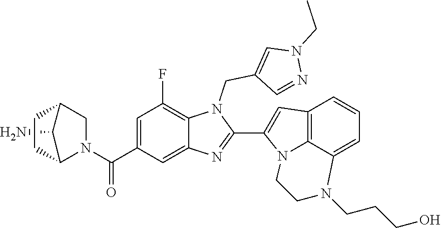 [SMILES: CCn1cc(Cn2c(-c3cc4cccc5c4n3CCN5CCCO)nc3cc(C(=O)N4C[C@H]5CC[C@@H]4[C@@H]5N)cc(F)c32)cn1]

Prepared in analogous manner as for Example 34. LC/MS(ESI⁺) [(M+H)⁺]: 596.7.

### Example 43 ((3R,5R)-3-amino-5-fluoropiperidin-1-yl)(2-(1-(3-hydroxypropyl)-2,3-dihydro-1H-pyrrolo[1,2,3-de]quinoxalin-5-yl)-7-methoxy-1-((1-methyl-1H-pyrazol-4-yl)methyl)-1H-benzo[d]imidazol-5-yl)methanone

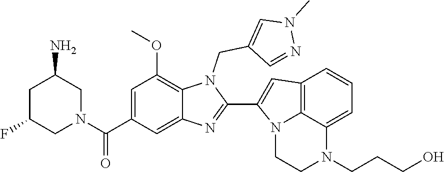 [SMILES: COc1cc(C(=O)N2C[C@H](N)C[C@@H](F)C2)cc2nc(-c3cc4cccc5c4n3CCN5CCCO)n(Cc3cnn(C)c3)c12]

Prepared in analogous manner as for Example 34. LC/MS(ESI⁺) [(M+H)⁺]: 600.8.

¹H NMR (400 MHz, DMSO-d₆) δ 7.42 (s, 1H), 7.32 (s, 1H), 7.10 (s, 1H), 6.99-6.90 (m, 3H), 6.87 (s, 1H), 6.43 (dd, J=6.2, 2.1 Hz, 1H), 5.70 (s, 2H), 4.50 (t, J=4.9 Hz, 2H), 3.98 (s, 3H), 3.73 (s, 3H), 3.60-3.50 (m, 5H), 3.48-3.39 (m, 7H), 3.01 (d, J=10.9 Hz, 1H), 2.18 (s, 1H), 1.81 (p, J=6.3 Hz, 2H), 1.63-1.49 (m, 1H).

### Example 44 (R)-(3-aminopiperidin-1-yl)(2-(1-(3-hydroxypropyl)-2,3-dihydro-1H-pyrrolo[1,2,3-de]quinoxalin-5-yl)-7-methoxy-1-((1-methyl-1H-pyrazol-4-yl)methyl)-1H-benzo[d]imidazol-5-yl)methanone

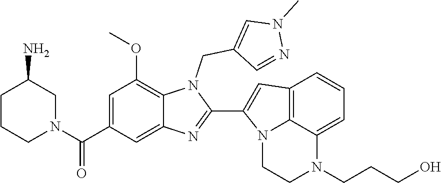 [SMILES: COc1cc(C(=O)N2CCC[C@@H](N)C2)cc2nc(-c3cc4cccc5c4n3CCN5CCCO)n(Cc3cnn(C)c3)c12]

Prepared in analogous manner as for Example 34. LC/MS(ESI⁺) [(M+H)⁺]: 582.8.

¹H NMR (400 MHz, DMSO-d₆) δ 8.30 (d, J=10.1 Hz, 1H), 7.65-7.59 (m, 1H), 7.51-7.47 (m, 2H), 7.38 (d, J=1.7 Hz, 1H), 7.12 (d, J=7.8 Hz, 1H), 7.04 (d, J=1.7 Hz, 1H), 6.99 (d, J=7.9 Hz, 1H), 6.96 (d, J=1.9 Hz, 1H), 6.43 (dd, J=6.7, 1.6 Hz, 1H), 4.64 (t, J=5.2 Hz, 2H), 4.22 (d, J=10.3 Hz, 3H), 4.02 (d, J=10.6 Hz, 3H), 3.58-3.50 (m, 8H), 2.88 (d, J=5.2 Hz, 1H), 2.42 (s, 1H), 2.30 (s, 2H), 1.80 (dd, J=17.1, 10.0 Hz, 4H), 1.41 (d, J=5.8 Hz, 2H).

### Example 45 ((1R,4R,7R)-7-amino-2-azabicyclo[2.2.1]heptan-2-yl)(2-(1-(3-hydroxypropyl)-2,3-dihydro-1H-pyrrolo[1,2,3-de]quinoxalin-5-yl)-7-methoxy-1-((1-methyl-1H-pyrazol-4-yl)methyl)-1H-benzo[d]imidazol-5-yl)methanone

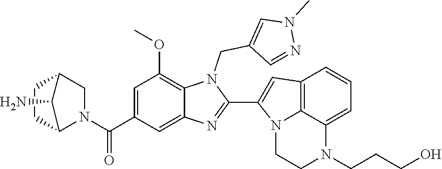 [SMILES: COc1cc(C(=O)N2C[C@H]3CC[C@@H]2[C@@H]3N)cc2nc(-c3cc4cccc5c4n3CCN5CCCO)n(Cc3cnn(C)c3)c12]

Prepared in analogous manner as for Example 34. LC/MS(ESI⁺) [(M+H)⁺]: 594.8.

### Example 46 ((3S,5S)-3-amino-5-fluoropiperidin-1-yl)(7-fluoro-2-(1-(3-hydroxypropyl)-2,3-dihydro-1H-pyrrolo[1,2,3-de]quinoxalin-5-yl)-1-((1-methyl-1H-pyrazol-4-yl)methyl)-1H-benzo[d]imidazol-5-yl)methanone

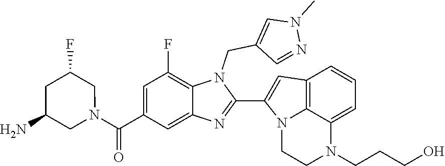 [SMILES: Cn1cc(Cn2c(-c3cc4cccc5c4n3CCN5CCCO)nc3cc(C(=O)N4C[C@@H](N)C[C@H](F)C4)cc(F)c32)cn1]

Prepared in analogous manner as for Example 34. LC/MS(ESI⁺) [(M+H)⁺]: 588.8.

¹H NMR (400 MHz, DMSO-d₆) δ 7.59-7.55 (m, 2H), 7.23 (s, 1H), 7.16 (d, J=11.9 Hz, 1H), 6.97-6.92 (m, 3H), 6.44 (dd, J=5.8, 2.5 Hz, 1H), 5.62 (s, 2H), 4.58 (t, J=5.2 Hz, 2H), 3.77 (s, 3H), 3.56 (q, J=5.4 Hz, 4H), 3.45 (t, J=7.4 Hz, 4H), 3.01 (d, J=11.0 Hz, 3H), 2.17 (s, 2H), 1.81 (p, J=6.3 Hz, 3H), 1.50 (s, 1H).

### Example 47 Synthesis report of ((3R,5R)-3-amino-5-fluoropiperidin-1-yl)(1-((1-ethyl-1H-pyrazol-4-yl)methyl)-7-fluoro-2-(1-(2,2,2-trifluoroethyl)-2,3-dihydro-1H-pyrrolo[1,2,3-de]quinoxalin-5-yl)-1H-benzo[d]imidazol-5-yl)methanone

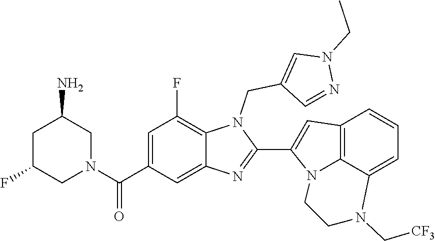 [SMILES: CCn1cc(Cn2c(-c3cc4cccc5c4n3CCN5CC(F)(F)F)nc3cc(C(=O)N4C[C@H](N)C[C@@H](F)C4)cc(F)c32)cn1]

Prepared in analogous manner as for Example 34. LC/MS(ESI⁺) [(M+H)⁺]: 626.7. ¹H NMR (400 MHz, DMSO-d₆) δ 8.26 (s, 1H), 7.59 (d, J=2.3 Hz, 2H), 7.24 (s, 1H), 7.17 (d, J=11.5 Hz, 1H), 7.08 (d, J=8.0 Hz, 1H), 6.99 (t, J=3.9 Hz, 2H), 6.63 (d, J=7.5 Hz, 1H), 5.62 (s, 2H), 4.58-4.53 (m, 2H), 4.30 (d, J=9.7 Hz, 2H), 4.05 (q, J=7.3 Hz, 2H), 3.75 (t, J=5.2 Hz, 2H), 2.98 (d, J=10.8 Hz, 2H), 2.68 (s, 1H), 2.34 (s, 1H), 2.15 (s, 2H), 1.61-1.45 (m, 2H), 1.29 (t, J=7.2 Hz, 3H).

### Example 48 Preparation of ((3R,5R)-3-amino-5-fluoropiperidin-1-yl)(1-(cyclobutylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

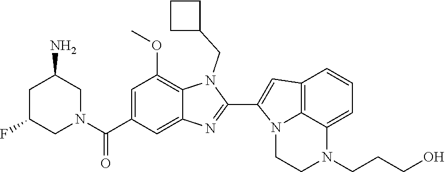 [SMILES: COc1cc(C(=O)N2C[C@H](N)C[C@@H](F)C2)cc2nc(-c3cc4cccc5c4n3CCN5CCCO)n(CC3CCC3)c12]

Prepared in analogous manner as for Example 34. LC-MS: (ESI) m/z 574.8 [M+H]⁺. ¹H NMR (400 MHz, DMSO-d₆) δ 8.27 (s, 1H), 7.31 (s, 1H), 6.95 (dd, J=9.4, 7.5 Hz, 2H), 6.91 (s, 1H), 6.86 (s, 1H), 6.41 (d, J=6.2 Hz, 1H), 4.72 (d, J=6.9 Hz, 2H), 4.46 (s, 2H), 4.00 (s, 3H), 3.62-3.50 (m, 12H), 3.08 (t, J=10.7 Hz, 1H), 2.65-2.57 (m, 1H), 2.21 (s, 1H), 1.78 (dd, J=16.4, 10.3 Hz, 4H), 1.72-1.60 (m, 2H), 1.51 (dd, J=18.5, 8.9 Hz, 2H).

### Example 49 Preparation of ((3R,5R)-3-amino-5-fluoropiperidin-1-yl)(2-(1-(3-hydroxypropyl)-2,3-dihydro-1H-pyrrolo[1,2,3-de]quinoxalin-5-yl)-7-methoxy-1-(prop-2-yn-1-yl)-1H-benzo[d]imidazol-5-yl)methanone

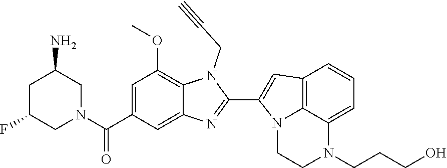 [SMILES: C#CCn1c(-c2cc3cccc4c3n2CCN4CCCO)nc2cc(C(=O)N3C[C@H](N)C[C@@H](F)C3)cc(OC)c21]

Prepared in analogous manner as for Example 34. LC-MS: (ESI) m/z 544.8 [M+H]⁺.

### Example 50 Preparation of ((3R,5R)-3-amino-5-fluoropiperidin-1-yl)(1-((3,3-difluorocyclobutyl)methyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

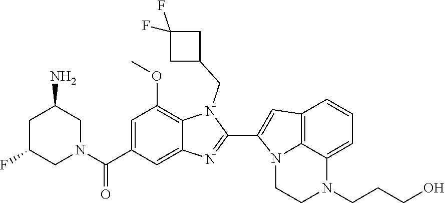 [SMILES: COc1cc(C(=O)N2C[C@H](N)C[C@@H](F)C2)cc2nc(-c3cc4cccc5c4n3CCN5CCCO)n(CC3CC(F)(F)C3)c12]

Prepared in analogous manner as for Example 34. LC-MS: (ESI) m/z 610.8 [M+H]⁺. ¹H NMR (400 MHz, DMSO-d₆) δ 8.27 (s, 1H), 7.32 (s, 1H), 6.94 (p, J=8.2 Hz, 3H), 6.88 (s, 1H), 6.42 (d, J=5.5 Hz, 1H), 4.85 (d, J=5.9 Hz, 2H), 4.48 (s, 2H), 4.00 (s, 3H), 3.54 (t, J=5.9 Hz, 4H), 3.49-3.39 (m, 8H), 3.03 (t, J=10.6 Hz, 1H), 2.57 (d, J=11.8 Hz, 1H), 2.29-2.11 (m, 3H), 1.87-1.74 (m, 2H), 1.71-1.39 (m, 2H).

### Example 51 Preparation of ((3R,5R)-3-amino-5-fluoropiperidin-1-yl)(1-(2,2-difluoroethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

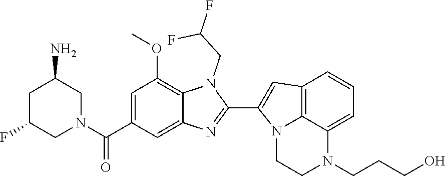 [SMILES: COc1cc(C(=O)N2C[C@H](N)C[C@@H](F)C2)cc2nc(-c3cc4cccc5c4n3CCN5CCCO)n(CC(F)F)c12]

Prepared in analogous manner as for Example 34. LC-MS: (ESI) m/z 570.8 [M+H]⁺.

### Example 52 (1-amino-5-azaspiro[2.4]heptan-5-yl)(1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

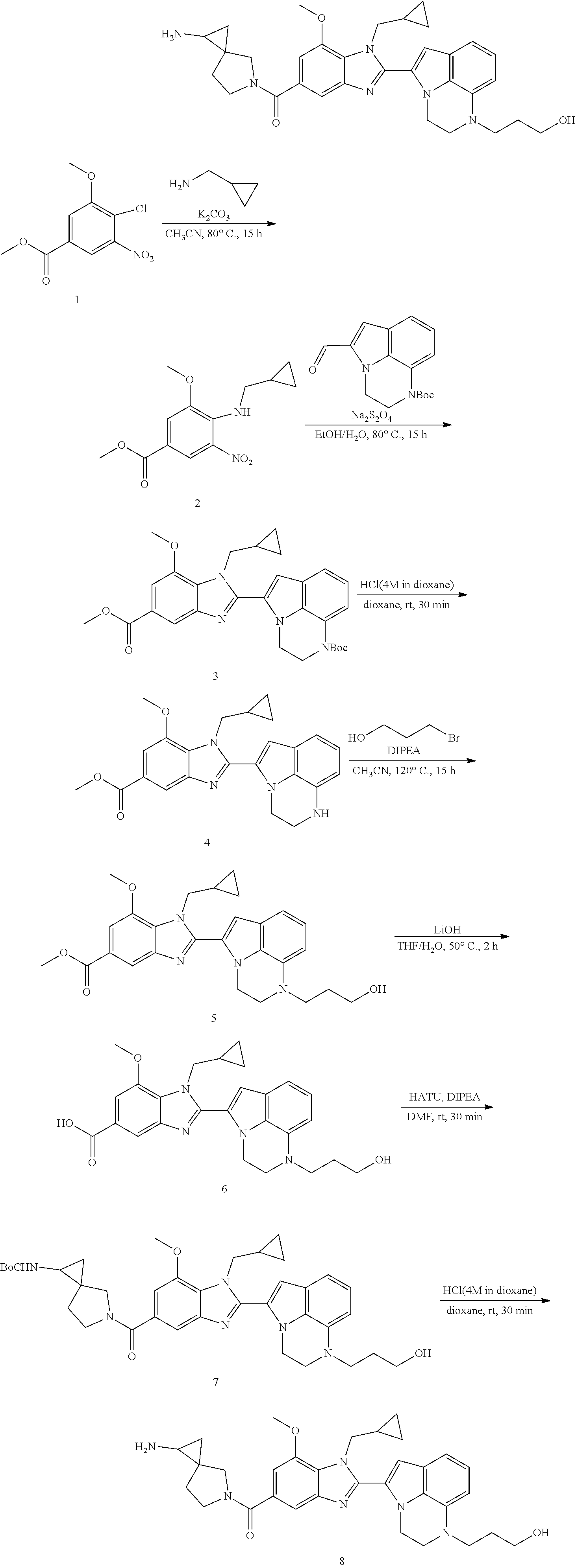

### Step 1:

A mixture of cyclopropylmethanamine (2.2 g, 30.54 mmol, 2.65 mL), methyl 4-chloro-3-methoxy-5-nitro-benzoate (5.0 g, 20.36 mmol) and potassium carbonate (5.6 g, 40.71 mmol, 2.46 mL) was dissolved in acetonitrile (30 mL). The resulting mixture was stirred at 80° C. for 15 h. Desired signal was found by LC/MS. The reaction was diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give methyl 4-(cyclopropylmethylamino)-3-methoxy-5-nitro-benzoate (4.0 g, 14.27 mmol, 70.11% yield) as a yellow solid. LC/MS(ESI⁺) [(M+H)]: 280.8.

### Step 2:

A mixture of methyl 4-(cyclopropylmethylamino)-3-methoxy-5-nitro-benzoate (4.0 g, 14.27 mmol), tert-butyl 3-formyl-1,9-diazatricyclo[6.3.1.04,12]dodeca-2,4(12), 5,7-tetraene-9-carboxylate (4.5 g, 15.70 mmol) sodium hydrosulfite (12.4 g, 71.36 mmol) was dissolved in ethanol (15 mL) and H₂O (15 mL). The resulting mixture was stirred at 80° C. for 15 h. The reaction was diluted with EtOAc (50 ml) and washed with water (25 ml). The organic layer was dried over sodium sulfate, filtered, and concentrated in vacuo. The crude material was purified by flash column chromatography on silica gel (eluting with EA/PE from 0 to 100%) to give tert-butyl 2-[1-(cyclopropylmethyl)-7-methoxy-5-methoxycarbonyl-benzimidazol-2-yl]-1,9-diazatricyclo[6.3.1.04,12]dodeca-2,4(12), 5,7-tetraene-9-carboxylate (5.1 g, 9.87 mmol, 69.18% yield) as a yellow solid. LC/MS (ESI⁺) [(M+H)⁺]: 516.8.

### Step 3:

Tert-butyl 2-[1-(cyclopropylmethyl)-7-methoxy-5-methoxycarbonyl-benzimidazol-2-yl]-1,9-diazatricyclo[6.3.1.04,12]dodeca-2,4(12), 5,7-tetraene-9-carboxylate (5.1 g, 9.87 mmol) was dissolved in HCl (4M)/Dioxane=1/2 (12 mL). The reaction solution was stirred at rt for 30 min. After removal of the solvent in vacuo, the crude product methyl 1-(cyclopropylmethyl)-2-(1,9-diazatricyclo[6.3.1.04,12]dodeca-2,4(12), 5,7-tetraen-2-yl)-7-methoxy-benzimidazole-5-carboxylate (3.8 g, 9.12 mmol, 92.42% yield) was obtained as a yellow solid. LC/MS(ESI⁺) [(M+H)⁺]: 416.8.

### Step 4:

A mixture of methyl 1-(cyclopropylmethyl)-2-(1,9-diazatricyclo[6.3.1.04,12]dodeca-2,4(12), 5,7-tetraen-2-yl)-7-methoxy-benzimidazole-5-carboxylate (3.8 g, 9.12 mmol), 3-bromopropan-1-ol (1.9 g, 13.69 mmol, 1.20 mL) and DIPEA (3.5 g, 27.37 mmol, 4.77 mL) was dissolved in acetonitrile (20 mL). The subsequent mixture was heated at 120° C. for 15 h in microwave reactor. After removal of the solvent in vacuo, the residue was purified by flash column chromatography on silica gel (eluting with EA/PE from 0 to 100) to give methyl 1-(cyclopropylmethyl)-2-[9-(3-hydroxypropyl)-1,9-diazatricyclo[6.3.1.04,12]dodeca-2,4(12), 5,7-tetraen-2-yl]-7-methoxy-benzimidazole-5-carboxylate (3.0 g, 6.32 mmol, 69.29% yield) as liquid oil. LC/MS (ESI⁺)[(M+H)⁺]: 474.8.

### Step 5:

To a solution of methyl 1-(cyclopropylmethyl)-2-[9-(3-hydroxypropyl)-1,9-diazatricyclo[6.3.1.04,12]dodeca-2,4(12), 5,7-tetraen-2-yl]-7-methoxy-benzimidazole-5-carboxylate (1.5 g, 3.16 mmol) in THF (9 mL) and H₂O (3 mL) was added lithium hydroxide hydrate (265.3 mg, 6.32 mmol, 175.67 μL). The resulting mixture was stirred at 100° C. for overnight. The mixture was acidified with 3 mol/L hydrochloric acid. The residue was purified by pre-HPLC to give 1-(cyclopropylmethyl)-2-[9-(3-hydroxypropyl)-1,9-diazatricyclo[6.3.1.04,12]dodeca-2,4(12), 5,7-tetraen-2-yl]-7-methoxy-benzimidazole-5-carboxylic acid (900.0 mg, 1.95 mmol, 61.83% yield) as a yellow solid. LC/MS(ESI⁺) [(M+H)⁺]: 460.8.

### Step 6:

A mixture of tert-butyl N-(5-azaspiro[2.4]heptan-2-yl)carbamate (15.2 mg, 71.66 μmol), 1-(cyclopropylmethyl)-2-[9-(3-hydroxypropyl)-1,9-diazatricyclo[6.3.1.04,12]dodeca-2,4(12), 5,7-tetraen-2-yl]-7-methoxy-benzimidazole-5-carboxylic acid (30.0 mg, 65.14 μmol), HATU (29.72 mg, 78.17 μmol) and DIPEA (12.6 mg, 97.71 μmol, 17.02 μL) was dissolved in DMF (2 mL). The resulting solution was stirred at 25° C. for 30 min, diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give tert-butyl N-[5-[1-(cyclopropylmethyl)-2-[9-(3-hydroxypropyl)-1,9-diazatricyclo[6.3.1.04,12]dodeca-2,4(12), 5,7-tetraen-2-yl]-7-methoxy-benzimidazole-5-carbonyl]-5-azaspiro[2.4]heptan-2-yl]carbamate (30.0 mg, 45.82 μmol, 70.33% yield) as a yellow solid. LC/MS(ESI⁺) [(M+H)⁺]: 654.8.

### Step 7:

Tert-butyl tert-butyl N-[5-[1-(cyclopropylmethyl)-2-[9-(3-hydroxypropyl)-1,9-diazatricyclo[6.3.1.04,12]dodeca-2,4(12), 5,7-tetraen-2-yl]-7-methoxy-benzimidazole-5-carbonyl]-5-azaspiro[2.4]heptan-2-yl]carbamate (30.0 mg, 45.82 μmol) was dissolved in HCl (4M)//Dioxane=1/2 (3 mL). The resulting mixture was stirred at RT for 30 min. After removal of the solvent in vacuo, the residue was purified by pre-HPLC to afford (2-amino-5-azaspiro[2.4]heptan-5-yl)-[1-(cyclopropylmethyl)-2-[9-(3-hydroxypropyl)-1,9-diazatricyclo[6.3.1.04,12]dodeca-2,4(12), 5,7-tetraen-2-yl]-7-methoxy-benzimidazol-5-yl]methanone (20.0 mg, 36.06 μmol, 78.70% yield) as a white solid. LC/MS(ESI⁺) [(M+H)⁺]: 554.8.

### The Following Compounds were Prepared Analogously

### Example 53 (3,6-diazabicyclo[3.2.0]heptan-3-yl)(1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

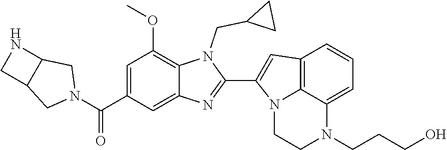 [SMILES: COc1cc(C(=O)N2CC3CNC3C2)cc2nc(-c3cc4cccc5c4n3CCN5CCCO)n(CC3CC3)c12]

Prepared in analogous manner as for Example 52. LC/MS(ESI⁺) [(M+H)⁺]: 540.8

### Example 54 (6-amino-3-azabicyclo[3.1.0]hexan-3-yl)(1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

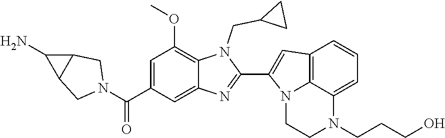 [SMILES: COc1cc(C(=O)N2CC3C(N)C3C2)cc2nc(-c3cc4cccc5c4n3CCN5CCCO)n(CC3CC3)c12]

Prepared in analogous manner as for Example 52. LC/MS(ESI⁺) [(M+H)⁺]: 540.8. ¹H NMR (400 MHz, DMSO-d₆) δ 7.29 (d, J=12.0 Hz, 1H), 6.87-6.77 (m, 4H), 6.30 (dd, J=6.4, 1.9 Hz, 1H), 4.44 (d, J=6.8 Hz, 2H), 4.37 (t, J=4.9 Hz, 2H), 3.88 (s, 3H), 3.86-3.79 (m, 1H), 3.62 (d, J=12.6 Hz, 2H), 3.47-3.40 (m, 5H), 3.35 (d, J=8.6 Hz, 4H), 1.91 (d, J=2.2 Hz, 1H), 1.75-1.62 (m, 2H), 1.49 (q, J=5.1 Hz, 1H), 1.41 (s, 1H), 1.03 (tq, J=8.1, 3.7 Hz, 1H), 0.24 (dt, J=8.3, 3.0 Hz, 4H).

### Example 55 (1-amino-3-azabicyclo[4.1.0]heptan-3-yl)(1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

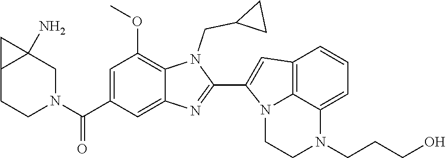 [SMILES: COc1cc(C(=O)N2CCC3CC3(N)C2)cc2nc(-c3cc4cccc5c4n3CCN5CCCO)n(CC3CC3)c12]

Prepared in analogous manner as for Example 52. LC/MS(ESI⁺) [(M+H)⁺]: 554.8. ¹H NMR (400 MHz, DMSO-d₆) δ 7.18 (s, 1H), 6.89-6.70 (m, 4H), 6.30 (dd, J=6.4, 1.9 Hz, 1H), 4.44 (d, J=6.8 Hz, 2H), 4.36 (t, J=4.8 Hz, 2H), 3.88 (s, 3H), 3.42 (t, J=5.8 Hz, 11H), 2.84 (s, 1H), 1.88 (s, 1H), 1.67 (dt, J=12.6, 6.2 Hz, 2H), 1.52 (s, 1H), 1.06-0.89 (m, 2H), 0.55 (s, 1H), 0.24 (dt, J=8.3, 3.0 Hz, 4H).

### Example 56 (1-amino-6-azaspiro[2.5]octan-6-yl)(1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

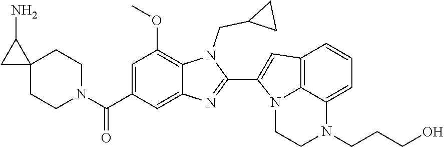 [SMILES: COc1cc(C(=O)N2CCC3(CC2)CC3N)cc2nc(-c3cc4cccc5c4n3CCN5CCCO)n(CC3CC3)c12]

Prepared in analogous manner as for Example 52. LC/MS(ESI⁺) [(M+H)⁺]: 568.7. ¹H NMR (400 MHz, DMSO-d₆) δ 7.20 (d, J=1.2 Hz, 1H), 6.86-6.75 (m, 4H), 6.30 (dd, J=6.4, 1.9 Hz, 1H), 4.44 (d, J=6.8 Hz, 2H), 4.36 (t, J=4.8 Hz, 2H), 3.89 (s, 3H), 3.44 (s, 3H), 3.41 (s, 4H), 3.32 (t, J=7.5 Hz, 4H), 2.06 (dd, J=7.4, 3.9 Hz, 1H), 1.68 (p, J=6.3 Hz, 2H), 1.60-1.41 (m, 2H), 1.33-1.11 (m, 2H), 1.07-1.00 (m, 1H), 0.40 (dd, J=7.3, 4.6 Hz, 1H), 0.29-0.21 (m, 2H), 0.08 (t, J=4.3 Hz, 3H).

### Example 57 (1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)(8,8-difluoro-2,6-diazaspiro[3.4]octan-6-yl)methanone

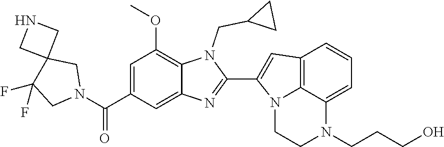 [SMILES: COc1cc(C(=O)N2CC(F)(F)C3(CNC3)C2)cc2nc(-c3cc4cccc5c4n3CCN5CCCO)n(CC3CC3)c12]

Prepared in analogous manner as for Example 52. LC/MS(ESI⁺) [(M+H)⁺]: 590.8. ¹H NMR (400 MHz, DMSO-d₆) δ 7.44 (s, 1H), 6.93-6.88 (m, 1H), 6.81 (d, J=6.3 Hz, 3H), 6.29 (dd, J=6.4, 1.9 Hz, 1H), 4.45 (d, J=6.8 Hz, 2H), 4.36 (t, J=4.8 Hz, 2H), 3.90 (s, 3H), 3.59 (s, 2H), 3.41 (t, J=6.0 Hz, 5H), 3.31 (t, J=7.4 Hz, 6H), 1.68 (p, J=6.3 Hz, 2H), 1.06-1.00 (m, 1H), 0.24 (dt, J=8.2, 3.0 Hz, 4H).

### Example 58 ((3R,4S)-3-amino-4-fluoropiperidin-1-yl)(1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

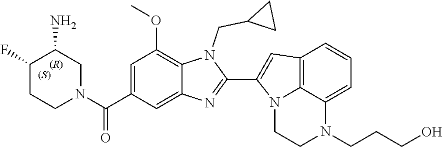

Prepared in analogous manner as for Example 52. LC/MS(ESI⁺) [(M+H)⁺]: 560.7

### Example 59 ((3R,5R)-3-amino-5-fluoropiperidin-1-yl)(1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

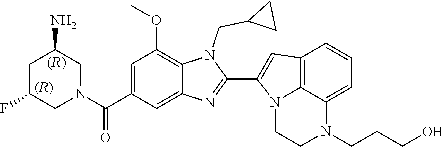

Prepared in analogous manner as for Example 52. LC/MS(ESI⁺) [(M+H)⁺]: 560.7.

### Example 60 Preparation of (R)-(7-amino-5-azaspiro[2.4]heptan-5-yl)(1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

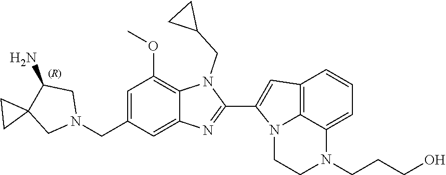

Prepared in analogous manner as for Example 52. LC/MS(ESI⁺) [(M+H)⁺]: 554.8. ¹H NMR (400 MHz, DMSO-d₆) δ 8.22 (s, 1H), 7.49 (s, 1H), 7.02 (s, 1H), 6.97-6.89 (m, 3H), 6.44-6.39 (m, 1H), 4.55 (s, 2H), 4.47 (s, 2H), 4.01 (s, 3H), 3.76-3.64 (m, 2H), 3.54 (t, J=5.7 Hz, 4H), 3.42 (d, J=7.2 Hz, 4H), 3.14 (s, 1H), 3.06 (s, 1H), 1.81 (dd, J=14.2, 6.5 Hz, 2H), 1.14 (s, 1H), 0.81 (s, 1H), 0.61 (s, 1H), 0.51 (s, 1H), 0.36 (d, J=7.5 Hz, 2H), 0.11 (d, J=4.7 Hz, 2H).

### Example 61 Preparation of (S)-(7-amino-5-azaspiro[2.4]heptan-5-yl)(1-(cyclopropylmethyl)-2-(1-(3-hydroxypropyl)-2,3-dihydro-1H-pyrrolo[1,2,3-de]quinoxalin-5-yl)-7-methoxy-1H-benzo[d]imidazol-5-yl)methanone

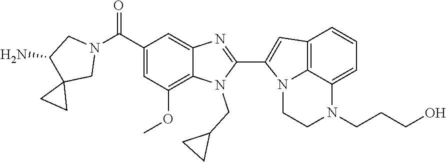 [SMILES: COc1cc(C(=O)N2C[C@@H](N)C3(CC3)C2)cc2nc(-c3cc4cccc5c4n3CCN5CCCO)n(CC3CC3)c12]

Prepared in analogous manner as for Example 52. LC-MS: (ESI) m/z 554.8 [M+H]⁺. ¹H NMR (400 MHz, DMSO-d₆) δ 8.23 (s, 1H), 7.49 (s, 1H), 7.02 (s, 1H), 6.98-6.87 (m, 3H), 6.41 (dd, J=6.4, 1.7 Hz, 1H), 4.55 (d, J=5.9 Hz, 2H), 4.47 (s, 2H), 4.01 (s, 3H), 3.79-3.63 (m, 2H), 3.54 (t, J=5.9 Hz, 4H), 3.44 (d, J=7.8 Hz, 4H), 3.15 (s, 1H), 3.07 (s, 1H), 1.84-1.75 (m, 2H), 1.14 (s, 1H), 0.79 (s, 1H), 0.62 (s, 1H), 0.51 (s, 1H), 0.36 (d, J=7.3 Hz, 2H), 0.11 (d, J=4.9 Hz, 2H).

### Example 62 Preparation of 3-(5-(5-(5-amino-4,5,6,7-tetrahydro-2H-indazol-2-yl)-7-methoxy-1-methyl-1H-benzo[d]imidazol-2-yl)-2,3-dihydro-1H-pyrrolo[1,2,3-de]quinoxalin-1-yl)propan-1-ol

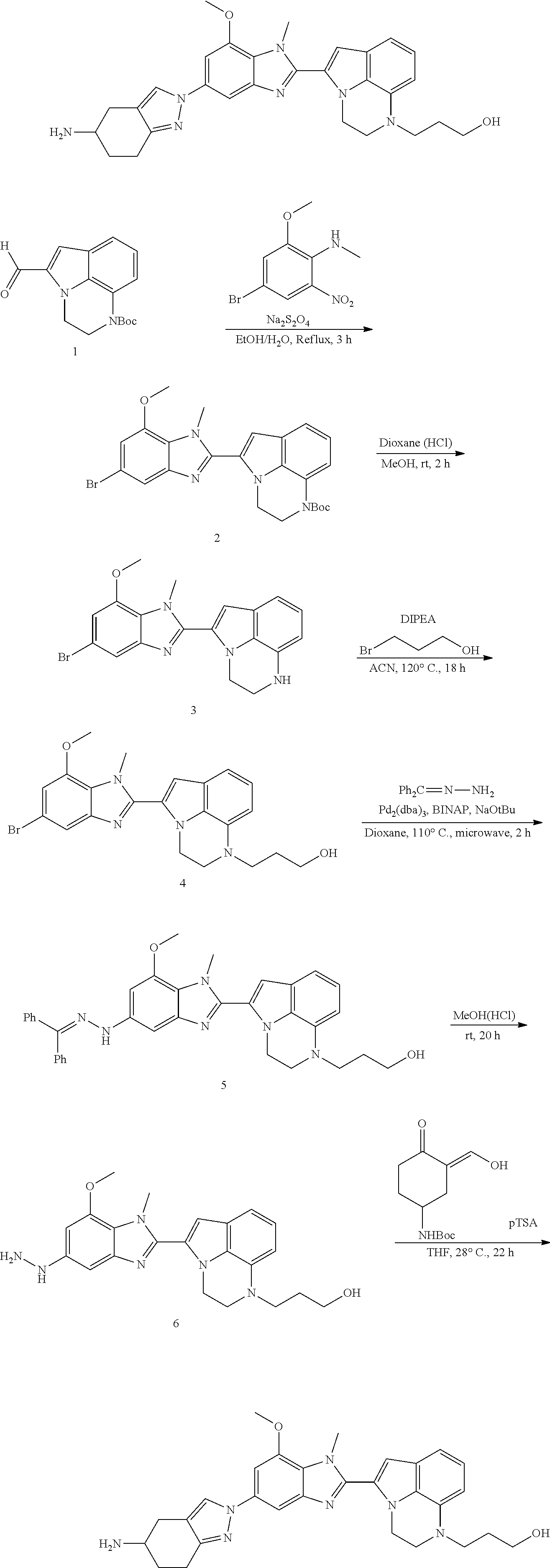

### Step 1:

To a solution of 4-bromo-2-methoxy-N-methyl-6-nitro-aniline (392.1 mg, 1.50 mmol) and tert-butyl 2-formyl-1,9-diazatricyclo[6.3.1.04,12]dodeca-2,4,6,8(12)-tetraene-9-carboxylate (0.43 g, 1.50 mmol) in EtOH/H₂O (9 mL/3 mL) was added Na₂S₂O₄ (784.43 mg, 4.51 mmol). The mixture was heated under reflux for 3 h. After the reaction was completed, the mixture was concentrated in vacuo and the residue was extracted with EtOAc (2*10 mL). Combined organic extracts were washed with brine (20 mL), dried over sodium sulfate and evaporated to give the crude product. The crude product was purified by flash column chromatography on silica gel using 1-40% EtOAc in hexane to afford title product tert-butyl 2-(5-bromo-7-methoxy-1-methyl-benzimidazol-2-yl)-1,9-diazatricyclo[6.3.1.04,12]dodeca-2,4(12), 5,7-tetraene-9-carboxylate (0.65 g, 1.31 mmol, 87.02% yield) as a bluish white solid. LC/MS (ESI⁺) [(M+H)⁺]: 496.8.

### Step 2:

To a stirred solution tert-butyl 2-(5-bromo-7-methoxy-1-methyl-benzimidazol-2-yl)-1,9-diazatricyclo[6.3.1.04,12]dodeca-2,4(12), 5,7-tetraene-9-carboxylate (0.65 g, 1.31 mmol) in MeOH (1 mL) was added 4M HCl in dioxane (10 mL) and the reaction mixture was stirred at RT for 2 h. The reaction mixture was evaporated to afford the product of 2-(5-bromo-7-methoxy-1-methyl-benzimidazol-2-yl)-1,9-diazatricyclo[6.3.1.04,12]dodeca-2,4(12), 5,7-tetraene (crude 0.6 g) as an off-white solid. LC/MS(ESI⁺) [(M+H)⁺]: 396.7.

### Step 3:

To a solution of 2-(5-bromo-7-methoxy-1-methyl-benzimidazol-2-yl)-1,9-diazatricyclo[6.3.1.04,12]dodeca-2,4(12), 5,7-tetraene (0.6 g, 1.51 mmol), 3-Bromo-1-propanol (1.05 g, 7.55 mmol, 660.12 μL) in acetonitrile (10 mL) was added DIPEA (975.97 mg, 7.55 mmol, 1.32 mL). The resulting mixture was heated at 120° C. in a sealed tube for 18 h. The reaction was allowed to cool to RT and concentrated in vacuo. The crude material was purified by flash column chromatography on silica gel (5-60% ethyl acetate/heptane) to obtain 3-[2-(5-bromo-7-methoxy-1-methyl-benzimidazol-2-yl)-1,9-diazatricyclo[6.3.1.04,12]dodeca-2,4(12), 5,7-tetraen-9-yl]propan-1-ol (250 mg, 549.03 μmol, 36.35% yield) as an off-white solid. LC/MS (ESI⁺) [(M+H)⁺]: 454.8.

### Step 4:

To a microwave tube containing 3-[2-(5-bromo-7-methoxy-1-methyl-benzimidazol-2-yl)-1,9-diazatricyclo[6.3.1.04,12]dodeca-2,4(12), 5,7-tetraen-9-yl]propan-1-ol (250 mg, 549.03 μmol), diphenylmethanone hydrazone (107.75 mg, 549.03 μmol, 97.95 μL) in dioxane (3 mL) were added Tris(Dibenzylideneacetone)dipalladium (0) (50.28 mg, 54.90 μmol), sodium tert-butoxide (73.87 mg, 768.65 μmol), and benzyl-[1-[2-[benzyl(phenyl)phosphanyl]-1-naphthyl]-2-naphthyl]-phenyl-phosphane (71.45 mg, 109.81 μmol). The resulting solution was degassed by N₂ gas balloon. The tube was then sealed and heated to 120° C. for 2 h. After the completion of the reaction, the mixture was cooled to RT and filtered through a pad of Celite, which was washed with ethyl acetate (3×10 mL). The combined solution was concentrated in vacuo to afford a yellowish solid. The crude product was purified by flash column chromatography on silica gel eluted with (EtOAc/petroleum ether, NA/100%-25%, v/v) to afford 3-[2-[5-(2-benzhydrylidenehydrazino)-7-methoxy-1-methyl-benzimidazol-2-yl]-1,9-diazatricyclo[6.3.1.04,12]dodeca-2,4(12), 5,7-tetraen-9-yl]propan-1-ol (160 mg, 280.37 μmol, 51.07% yield) as a grey white solid. LC/MS (ESI⁺) [(M+H)⁺]: 570.8.

### Step 5:

To a stirred solution 3-[2-[5-(2-benzhydrylidenehydrazino)-7-methoxy-1-methyl-benzimidazol-2-yl]-1,9-diazatricyclo[6.3.1.04,12]dodeca-2,4(12), 5,7-tetraen-9-yl]propan-1-ol (160 mg, 280.37 μmol) in MeOH (1 mL) was added 4 M HCl in MeOH (8 mL) and the reaction mixture was stirred at RT for 20 h. The reaction mixture was evaporated to afford the product 3-[2-(5-hydrazino-7-methoxy-1-methyl-benzimidazol-2-yl)-1,9-diazatricyclo[6.3.1.04,12]dodeca-2,4(12), 5,7-tetraen-9-yl]propan-1-ol (100 mg, crude) as an off-white solid. LC/MS (ESI⁺) [(M+H)⁺]: 406.8.

### Step 6:

To a stirred solution of tert-butyl N-[(3Z)-3-(hydroxymethylene)-4-oxo-cyclohexyl]carbamate (71.23 mg, 295.22 μmol) in THF (6 mL) at RT was added pTSA (127.09 mg, 738.04 μmol). The reaction mixture was stirred at RT for 22 h and upon completion of the reaction, the mixture was diluted with EtOAc (20 mL) and concentrated in vacuo. The crude product was purified by flash column chromatography on silica gel (1-20% MeOH/CH₂Cl₂), then prep-HPLC to afford desired product 3-[2-[5-(5-amino-4,5,6,7-tetrahydroindazol-2-yl)-7-methoxy-1-methyl-benzimidazol-2-yl]-1,9-diazatricyclo[6.3.1.04,12]dodeca-2,4(12), 5,7-tetraen-9-yl]propan-1-ol (4.5 mg, 8.80 μmol, 3.58% yield) as a white solid. LC/MS (ESI⁺) [(M+H)⁺]: 511.8.

### Example 63 Preparation of ((3R,5R)-3-amino-5-fluoropiperidin-1-yl)(2-(1-(3-hydroxypropyl)-2,3-dihydro-1H-pyrrolo[1,2,3-de]quinoxalin-5-yl)-1,7-dimethoxy-1H-benzo[d]imidazol-5-yl)methanone

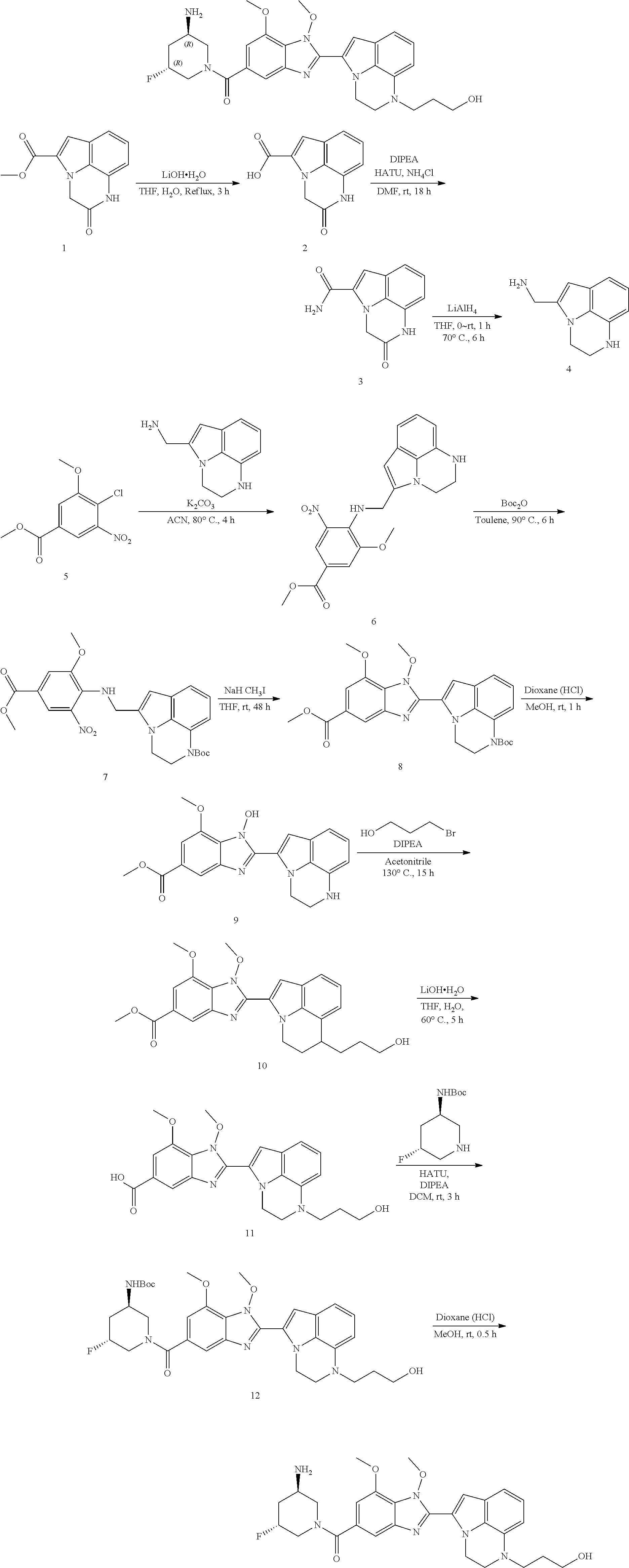

### Step 1:

To a solution of methyl 10-oxo-1,9-diazatricyclo[6.3.1.04,12]dodeca-2,4(12), 5,7-tetraene-2-carboxylate (5.1 g, 22.15 mmol) in THF (60 mL) was added a solution of Lithium hydroxide monohydrate, 98% (2.79 g, 66.46 mmol) in water (20 mL) and the resulting mixture was stirred under nitrogen at 70° C. for 3 h. The reaction crude was concentrated in vacuo and taken up in water (10 mL), acidified with 2N aqueous hydrochloric acid until no further precipitation was observed. The resulting suspension was allowed to stir for 30 min and filtered through filter paper. The resulting solid was dried to afford 10-oxo-1,9-diazatricyclo[6.3.1.04,12]dodeca-2,4(12), 5,7-tetraene-2-carboxylic acid (4.75 g, 21.97 mmol, 99.18% yield) as a brown solid. LC/MS(ESI⁺) [(M+H)⁺]: 216.7.

### Step 2:

To a solution of 10-oxo-1,9-diazatricyclo[6.3.1.04,12]dodeca-2,4(12), 5,7-tetraene-2-carboxylic acid (4.75 g, 21.97 mmol) and ammonium chloride (3.53 g, 65.91 mmol, 2.30 mL) in DMF (100 mL) at rt was added HATU (12.53 g, 32.96 mmol) and N,N-diisopropylethylamine (14.20 g, 109.86 mmol, 19.13 mL). The reaction mixture was stirred at rt for 16 h. After complecation of the reaction, the mixture was quenched with H₂O (250 mL) and some solid was formed, continued to stir for 0.5 h. The mixture was filtered and dried to afford title product 10-oxo-1,9-diazatricyclo[6.3.1.04,12]dodeca-2,4(12), 5,7-tetraene-2-carboxamide (2.3 g, 10.69 mmol, 48.64% yield) as a wheat solid. LC/MS (ESI⁺) [(M+H)⁺]: 215.8. The intermediate was directly used for next step without further purification.

### Step 3:

To a solution of 10-oxo-1,9-diazatricyclo[6.3.1.04,12]dodeca-2,4(12), 5,7-tetraene-2-carboxamide (2.3 g, 10.69 mmol) in THF (50 mL) was added Lithium Aluminum Hydride (1.09 g, 32.06 mmol) in portions at 0° C. The mixture was stirred at 70° C. for 6 h. The reaction mixture was cooled down to 0-5° C., quenched with water (1 mL) follow by 15% NaOH (aq) (1 mL), then water (3 mL) and dried over sodium sulfate. The mixture was filtered and concentrated in vacuo to afford 1,9-diazatricyclo[6.3.1.04,12]dodeca-2,4(12), 5,7-tetraen-2-ylmethanamine (1.65 g, 8.81 mmol, 82.45% yield) as brown oil. LC/MS(ESI⁺) [(M+H)⁺]: 187.8.

### Step 4:

To a stirred solution of methyl 4-chloro-3-methoxy-5-nitro-benzoate (1.0 g, 4.07 mmol) in Acetonitrile, (15 mL) were added 1,9-diazatricyclo[6.3.1.04,12]dodeca-2,4(12), 5,7-tetraen-2-ylmethanamine (800.5 mg, 4.27 mmol) and potassium carbonate (844.1 mg, 6.11 mmol), the mixture was stirred in sealed tube at 80° C. for 4 h. The reaction crude was filtered and concentrated in vacuo. The residue was purified by flash column chromatography on silica gel (1-60% ethyl acetate/heptane) to afford methyl 4-(1,9-diazatricyclo[6.3.1.04,12]dodeca-2,4(12), 5,7-tetraen-2-ylmethylamino)-3-methoxy-5-nitro-benzoate (0.6 g, 1.51 mmol, 37.18% yield) as a brown solid. LC/MS (ESI⁺) [(M+H)⁺]: 396.8.

### Step 5:

To a solution of methyl 4-(1,9-diazatricyclo[6.3.1.04,12]dodeca-2,4(12), 5,7-tetraen-2-ylmethylamino)-3-methoxy-5-nitro-benzoate (0.6 g, 1.51 mmol) in Toluene, (12 mL) was added Di-tert-butyl dicarbonate (991.1 mg, 4.54 mmol, 1.04 mL). The resulting mixture was stirred at 90° C. for 6 h. The reaction mixture was cooled to RT, concentrated in vacuo. The crude product was purified by flash column chromatography on silica gel (0-20% ethyl acetate/heptane) to obtain tert-butyl 2-[(2-methoxy-4-methoxycarbonyl-6-nitro-anilino)methyl]-1,9-diazatricyclo[6.3.1.04,12]dodeca-2,4(12), 5,7-tetraene-9-carboxylate (0.26 g, 523.65 μmol, 34.60% yield) as a brown solid. LC/MS(ESI⁺) [(M+H)⁺]: 496.7.

### Step 6:

To a stirred solution of tert-butyl 2-[(2-methoxy-4-methoxycarbonyl-6-nitro-anilino)methyl]-1,9-diazatricyclo[6.3.1.04,12]dodeca-2,4(12), 5,7-tetraene-9-carboxylate (0.26 g, 523.65 μmol) in THF (6 mL) at 0-5° C. was added sodium hydride 60% dispersion in mineral oil (37.7 mg, 1.57 mmol) and stirred for 0.5 h. Iodomethane (148.7 mg, 1.05 mmol, 65.20 μL) was added to the mixture and stirred at RT for 48 h. After the reaction was completed, the mixture was quenched with H₂O (8 mL) and diluted with EtOAc (25 mL) and warmed to RT. The layers were separated, and the aqueous layer was extracted with EtOAc (15 mL*2). The combined organic phase was washed with brine (30 mL) and dried over anhydrous Na₂SO₄, filtered and concentrated in vacuo. The crude product was purified by flash column chromatography on silica gel (5-50% ethyl acetate/heptane) to afford desired product tert-butyl 2-(1-hydroxy-7-methoxy-5-methoxycarbonyl-benzimidazol-2-yl)-1,9-diazatricyclo[6.3.1.04,12]dodeca-2,4(12), 5,7-tetraene-9-carboxylate (80 mg, 167.19 μmol, 31.93% yield), tert-butyl 2-(1,7-dimethoxy-5-methoxycarbonyl-benzimidazol-2-yl)-1,9-diazatricyclo[6.3.1.04,12]dodeca-2,4(12), 5,7-tetraene-9-carboxylate (10 mg, 20.30 μmol, 3.88% yield) as a white solid. LC/MS (ESI⁺) [(M+H)⁺]: 478.8, 492.8.

### Step 7:

To a stirred solution tert-butyl 2-(1-hydroxy-7-methoxy-5-methoxycarbonyl-benzimidazol-2-yl)-1,9-diazatricyclo[6.3.1.04,12]dodeca-2,4(12), 5,7-tetraene-9-carboxylate (80 mg, 167.19 μmol) in MeOH (0.5 mL) was added 4M HCl in dioxane (2 mL) and the reaction mixture was stirred at RT for 1 h. The reaction mixture was evaporated to afford the product methyl 2-(1,9-diazatricyclo[6.3.1.04,12]dodeca-2,4(12), 5,7-tetraen-2-yl)-1-hydroxy-7-methoxy-benzimidazole-5-carboxylate (70 mg, 185.00 μmol, 110.65% yield) as an off-white solid. LC/MS (ESI⁺) [(M+H)⁺]: 378.8.

### Step 8:

To a solution of methyl 2-(1,9-diazatricyclo[6.3.1.04,12]dodeca-2,4(12), 5,7-tetraen-2-yl)-1-hydroxy-7-methoxy-benzimidazole-5-carboxylate (80 mg, 192.84 μmol, HC), 3-Bromo-1-propanol (134.02 mg, 964.22 μmol, 84.29 μL) in acetonitrile (2 mL) was added N,N-Diisopropylethylamine (124.62 mg, 964.22 μmol, 167.95 μL). The resulting mixture was heated to 130° C. in a sealed tube for 15 h. The reaction was allowed to cool to RT and concentrated in vacuo. The crude material was purified by flash column chromatography on silica gel (5-60% ethyl acetate/heptane) to obtain methyl 1-(3-hydroxypropoxy)-2-[9-(3-hydroxypropyl)-1,9-diazatricyclo[6.3.1.04,12]dodeca-2,4(12), 5,7-tetraen-2-yl]-7-methoxy-benzimidazole-5-carboxylate (70 mg, 141.55 μmol, 73.40% yield), methyl 2-[9-(3-hydroxypropyl)-1,9-diazatricyclo[6.3.1.04,12]dodeca-2,4(12), 5,7-tetraen-2-yl]-1,7-dimethoxy-benzimidazole-5-carboxylate (8 mg, 17.76 μmol, 9.21% yield). LC/MS(ESI⁺) [(M+H)⁺]: 450.8.

### Step 9:

To a solution of methyl 1-(3-hydroxypropoxy)-2-[9-(3-hydroxypropyl)-1,9-diazatricyclo[6.3.1.04,12]dodeca-2,4(12), 5,7-tetraen-2-yl]-7-methoxy-benzimidazole-5-carboxylate (70 mg, 141.55 μmol), methyl 2-[9-(3-hydroxypropyl)-1,9-diazatricyclo[6.3.1.04,12]dodeca-2,4(12), 5,7-tetraen-2-yl]-1,7-dimethoxy-benzimidazole-5-carboxylate (8 mg, 17.76 μmol) in THF (3.0 mL) was added a solution of lithium hydroxide monohydrate, 98% (17.82 mg, 424.64 μmol) in water (0.5 mL) and the resulting mixture was stirred at 60° C. for 5 h. The reaction crude was concentrated in vacuo and taken up in water (5 mL), acidified with 2N aqueous hydrochloric acid, then extracted with EtOAc (15 mL*2). Organic layer was separated, washed with brine (10 mL) solution, dried over anhydrous Na₂SO₄ and evaporated under vacuum to give the product 2-[9-(3-hydroxypropyl)-1,9-diazatricyclo[6.3.1.04,12]dodeca-2,4(12), 5,7-tetraen-2-yl]-1,7-dimethoxy-benzimidazole-5-carboxylic acid (20 mg, 45.82 μmol, 32.37% yield). LC/MS(ESI⁺) [(M+H)⁺]: 436.7.

### Step 10:

To a solution of 2-[9-(3-hydroxypropyl)-1,9-diazatricyclo[6.3.1.04,12]dodeca-2,4(12), 5,7-tetraen-2-yl]-1,7-dimethoxy-benzimidazole-5-carboxylic acid (20 mg, 45.82 μmol) and tert-butyl N-[(3R,5R)-5-fluoro-3-piperidyl]carbamate (10.50 mg, 48.11 μmol) in CH₂Cl₂ (3 mL)) at rt was added HATU (22.65 mg, 59.57 μmol) and N,N-Diisopropylethylamine (17.77 mg, 137.47 μmol, 23.94 μL). The reaction mixture was stirred at rt for 3 h. After the reaction was completed, quenched with H₂O (8 mL) and extracted with CH₂Cl₂ (2*20 mL). Combined organic extracts were washed with brine (20 mL), dried over sodium sulfate, and evaporated to give the crude product. The crude product was purified by flash column chromatography on silica gel using 2-20% MeOH in CH₂Cl₂ to afford title product tert-butyl N-[(3R,5R)-5-fluoro-1-[2-[9-(3-hydroxypropyl)-1,9-diazatricyclo[6.3.1.04,12]dodeca-2,4(12), 5,7-tetraen-2-yl]-1,7-dimethoxy-benzimidazole-5-carbonyl]-3-piperidyl]carbamate (15 mg, 23.56 μmol, 51.41% yield) as a bluish white solid. LC/MS (ESI⁺) [(M+H)⁺]: 636.8.

### Step 11:

To a stirred solution tert-butyl N-[(3R,5R)-5-fluoro-1-[2-[9-(3-hydroxypropyl)-1,9-diazatricyclo[6.3.1.04,12]dodeca-2,4(12), 5,7-tetraen-2-yl]-1,7-dimethoxy-benzimidazole-5-carbonyl]-3-piperidyl]carbamate (15 mg, 23.56 μmol) in MeOH (0.5 mL) was added 4M HCl in dioxane (2 mL) and the reaction mixture was stirred at RT for 0.5 h. The reaction mixture was evaporated to afford the crude product and then prep-HPLC to give [(3R,5R)-3-amino-5-fluoro-1-piperidyl]-[2-[9-(3-hydroxypropyl)-1,9-diazatricyclo[6.3.1.04,12]dodeca-2,4(12), 5,7-tetraen-2-yl]-1,7-dimethoxy-benzimidazol-5-yl]methanone (6 mg, 11.18 μmol, 47.46% yield) as a white solid. LC/MS (ESI⁺) [(M+H)⁺]: 536.8.

### Example 64 Preparation of ((3R,5R)-3-amino-5-fluoropiperidin-1-yl)(2-(1-(3-hydroxypropyl)-3-methyl-2,3-dihydro-1H-pyrrolo[1,2,3-de]quinoxalin-5-yl)-7-methoxy-1-methyl-1H-benzo[d]imidazol-5-yl)methanone

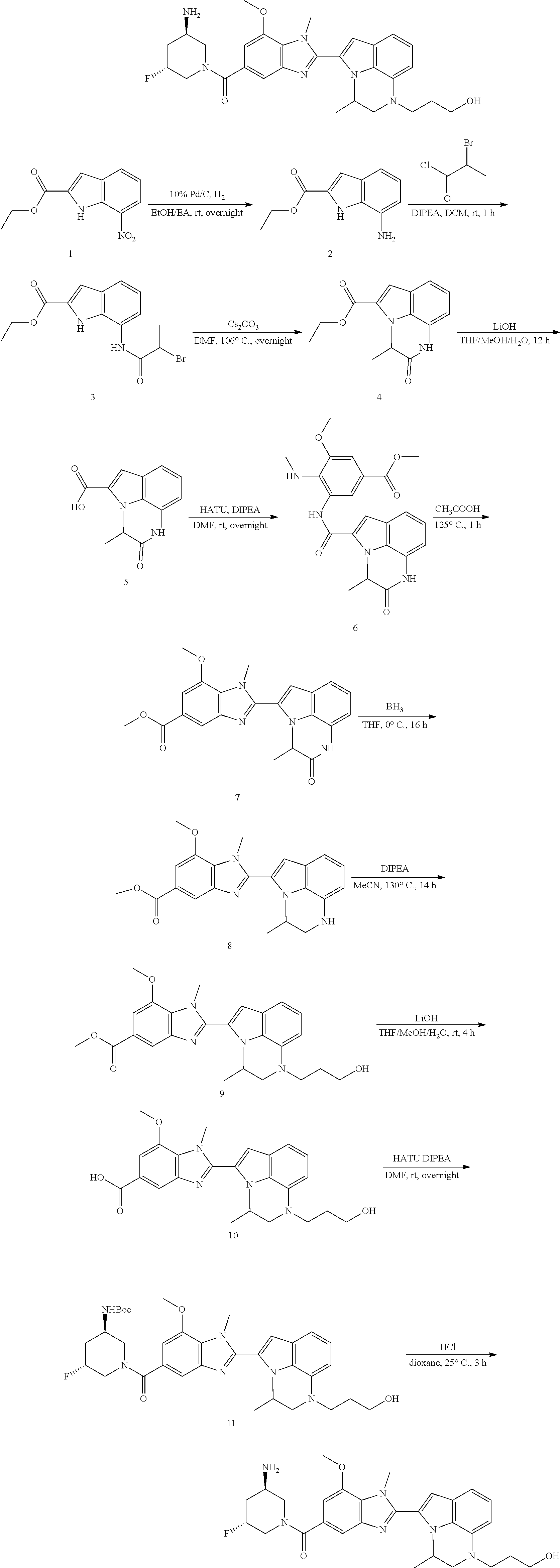

### Step 1:

To a solution of ethyl 7-nitro-1H-indole-2-carboxylate (10.0 g, 42.70 mmol) in ethanol (200 mL) and ethyl acetate (200 mL) was added 10% palladium on carbon (599.6 mg, 4.27 mmol) at RT. The reaction mixture was stirred at RT under H₂ atmosphere 16 h and filtered. The filtrate was concentrated in vacuo to afford ethyl 7-amino-1H-indole-2-carboxylate (8.8 g, 43.09 mmol, 100.92% yield) as a yellow solid. LC/MS(ESI⁺) [(M+H)⁺]: 204.8.

### Step 2:

To a solution of ethyl 7-amino-1H-indole-2-carboxylate (5.1 g, 24.97 mmol) in DCM (50 mL) was added N, N-Diisopropylethylamine (9.68 g, 74.92 mmol, 13.05 mL) and 2-bromopropanoyl chloride (12.84 g, 74.92 mmol) at rt. The reaction mixture was stirred at rt for 1 h, diluted with DCM (300 mL) and washed with water (20 mL). The organic phase was dried over anhydrous sodium sulfate and filtered. The filtrate was concentrated in vacuo, and the residue was purified by flash column chromatography on silica gel (EA/PE: 1/3, Rf=0.55) to provide ethyl 7-(2-bromopropanoylamino)-1H-indole-2-carboxylate (7.4 g, 21.82 mmol, 87.36% yield) as brown oil. LC/MS (ESI⁺) [(M+H)⁺]: 339.8.

### Step 3:

To a solution of ethyl 7-(2-bromopropanoylamino)-1H-indole-2-carboxylate (7.4 g, 21.82 mmol) in DMF (140 mL) was added dicesium carbonate (21.33 g, 65.45 mmol) at RT. The reaction mixture was stirred at 106° C. for 16 h. Quenched with ice water (100 mL) and extracted with ethyl acetate (100 mL*3). The organic phase was washed with brine (50 mL×3) and dried over anhydrous sodium sulfate and filtered. The filtrate was concentrated in vacuo, and the residue was purified by flash column chromatography on silica gel (PE/EA=1/0-1/1) to provide ethyl 11-methyl-10-oxo-1,9-diazatricyclo[6.3.1.04,12]dodeca-2,4(12), 5,7-tetraene-2-carboxylate (4.0 g, 15.49 mmol, 70.99% yield) as a white solid, LC/MS (ESI⁺) [(M+H)⁺]: 258.8.

### Step 4:

To a stirred solution of ethyl 11-methyl-10-oxo-1,9-diazatricyclo[6.3.1.04,12]dodeca-2,4(12), 5,7-tetraene-2-carboxylate (2.0 g, 7.74 mmol) in THF (20 mL) MeOH (10 mL) was added LiOH aqueous solution (1.0 M, 23 mL). The mixture was stirred at RT for 12 h, acidified to pH 5-6 with 3M hydrochloric acid aqueous solution, and extracted with EA (100*3 mL). The organic phase was dried over anhydrous sodium sulfate and filtered. The filtrate was concentrated in vacuo to give white solid 11-methyl-10-oxo-1,9-diazatricyclo[6.3.1.04,12]dodeca-2,4(12), 5,7-tetraene-2-carboxylic acid (1.2 g, 5.21 mmol, 67.31% yield), LC/MS (ESI⁺) [(M+H)⁺]: 229.8.

Step 5: To a solution of 11-methyl-10-oxo-1,9-diazatricyclo[6.3.1.04,12]dodeca-2,4(12), 5,7-tetraene-2-carboxylic acid (0.5 g, 2.17 mmol) in DMF (10 mL) were added DIPEA (842.07 mg, 6.52 mmol, 1.13 mL), HATU (1.65 g, 4.34 mmol) and methyl 3-amino-5-methoxy-4-(methylamino)benzoate (913.2 mg, 4.34 mmol). The resulting mixture was stirred at RT 16 h. LC-MS showed the starting material was consumed and the desired mass was detected. After cooling to RT, the mixture was diluted with EA and washed with brine and dried over anhydrous sodium sulfate. After filtration and evaporation of the solvent in vacuo, and the residue was purified by silica gel flash column chromatography (eluting with DCM/MeOH=1:0-20:1) to give methyl 3-methoxy-4-(methylamino)-5-[(11-methyl-10-oxo-1,9-diazatricyclo[6.3.1.04,12]dodeca-2,4(12), 5,7-tetraene-2-carbonyl)amino]benzoate (0.8 g, 1.89 mmol, 87.20% yield) as a yellow solid, LC/MS (ESI⁺) [(M+H)⁺]: 422.8.

### Step 6:

A solution of methyl 3-methoxy-4-(methylamino)-5-[(11-methyl-10-oxo-1,9-diazatricyclo[6.3.1.04,12]dodeca-2,4(12), 5,7-tetraene-2-carbonyl)amino]benzoate (0.8 g, 1.89 mmol) in CH₃COOH (8 mL) was stirred at 125° C. for 1 h. The mixture was concentrated in vacuo, diluted with EA (80 mL) and washed with a solution of aqueous sodium bicarbonate and dried over Na₂SO₄. After filtration and evaporation of the solvent in vacuo, the residue was purified by silica gel flash column chromatography (eluting with DCM/MeOH=1:0-20:1) to give methyl 7-methoxy-1-methyl-2-(11-methyl-10-oxo-1,9-diazatricyclo[6.3.1.04,12]dodeca-2,4(12), 5,7-tetraen-2-yl)benzimidazole-5-carboxylate (320 mg, 791.26 μmol, 41.78% yield) as a yellow solid, LC/MS (ESI⁺) [(M+H)⁺]: 403.8.

### Step 7:

To a solution of methyl 7-methoxy-1-methyl-2-(11-methyl-10-oxo-1,9-diazatricyclo[6.3.1.04,12]dodeca-2,4(12), 5,7-tetraen-2-yl)benzimidazole-5-carboxylate (320 mg, 791.26 μmol) in anhydrous THF (3 mL) was added borane tetrahydrofuran (272.0 mg, 3.17 mmol, 309.80 μL) at 0° C. slowly. The reaction mixture was stirred at RT for 16 h, quenched with MeOH at 0° C. and concentrated in vacuo. The residue was diluted with 2N HCl aqueous solution (6 mL), stirred at RT for 1 h, and basified with 4N NaOH aqueous solution to pH 8. The resulting mixture was extracted with DCM (30 mL*3), and the combined organic phase was dried over anhydrous sodium sulfate and filtered. The filtrate was concentrated in vacuo and the residue was purified by silica gel flash column chromatography (eluting with DCM/MeOH=1:0-20:1) to give methyl 7-methoxy-1-methyl-2-(11-methyl-1,9-diazatricyclo[6.3.1.04,12]dodeca-2,4(12), 5,7-tetraen-2-yl)benzimidazole-5-carboxylate (95 mg, 243.32 μmol, 30.75% yield) as a yellow solid. LC/MS (ESI⁺) [(M+H)⁺]: 390.8.

### Step 8:

To a solution of methyl 7-methoxy-1-methyl-2-(11-methyl-1,9-diazatricyclo[6.3.1.0^{4,12}]dodeca-2,4(12), 5,7-tetraen-2-yl)benzimidazole-5-carboxylate (65 mg, 166.48 μmol) in anhydrous ACN (3 mL) were added DIPEA (107.6 mg, 832.41 μmol, 144.99 μL) and 3-bromopropan-1-ol (115.7 mg, 832.41 μmol, 72.77 μL) at rt. The reaction mixture was stirred at 130° C. for 14 h with the microwave, cooled to rt and concentrated in vacuo, and the residue was purified by flash column chromatography on silica gel (elution with DCM/MeOH=1:0-20:1) to provide methyl 2-[9-(3-hydroxypropyl)-11-methyl-1,9-diazatricyclo[6.3.1.0^{4,12}]dodeca-2,4(12), 5,7-tetraen-2-yl]-7-methoxy-1-methyl-benzimidazole-5-carboxylate (20 mg, 44.59 μmol, 26.78% yield) as a yellow solid, LC/MS(ESI⁺) [(M+H)⁺]: 448.8.

### Step 9:

To a stirred solution of methyl 2-[9-(3-hydroxypropyl)-11-methyl-1,9-diazatricyclo[6.3.1.0^{4,12}]dodeca-2,4(12), 5,7-tetraen-2-yl]-7-methoxy-1-methyl-benzimidazole-5-carboxylate (20 mg, 44.59 μmol) in THF (2 mL) MeOH (1.0 mL) was added LiOH aqueous solution (1.0 M, 0.2 mL). The mixture was stirred at RT for 4 h, acidified to pH 5-6 with 3 M hydrochloric acid aqueous solution, and extracted with EA (10*3 mL). The organic phase was dried over anhydrous sodium sulfate and filtered. The filtrate was concentrated in vacuo to give 2-[9-(3-hydroxypropyl)-11-methyl-1,9-diazatricyclo[6.3.1.0^{4,12}]dodeca-2,4(12), 5,7-tetraen-2-yl]-7-methoxy-1-methyl-benzimidazole-5-carboxylic acid (15 mg, 34.52 μmol, 77.42% yield) 2-[9-(3-hydroxypropyl)-11-methyl-1,9-diazatricyclo[6.3.1.0^{4,12}]dodeca-2,4(12), 5,7-tetraen-2-yl]-7-methoxy-1-methyl-benzimidazole-5-carboxylic acid (15 mg, 34.52 μmol, 77.42% yield) as a white solid. LC/MS (ESI⁺) [(M+H)⁺]: 434.8.

### Step 10:

To a solution of 2-[9-(3-hydroxypropyl)-11-methyl-1,9-diazatricyclo[6.3.1.04,12]dodeca-2,4(12), 5,7-tetraen-2-yl]-7-methoxy-1-methyl-benzimidazole-5-carboxylic acid (15 mg, 34.52 μmol) in DMF (2 mL), were added DIPEA (13.4 mg, 103.57 μmol, 18.04 μL), HATU (26.3 mg, 69.05 μmol), tert-butyl N-[(3R,5R)-5-fluoro-3-piperidyl]carbamate (15.1 mg, 69.05 μmol) was added into the mixture. The resulting mixture was stirred at RT 16 h. After cooling to RT, the reaction mixture was diluted with EA and washed with brine and dried over anhydrous sodium sulfate. After filtration and evaporation of the solvent in vacuo, and the residue was purified by silica gel flash column chromatography (elution with DCM/MeOH=1:0-15:1) to give tert-butyl N-[(3R,5R)-5-fluoro-1-[2-[9-(3-hydroxypropyl)-11-methyl-1,9-diazatricyclo[6.3.1.04,12]dodeca-2,4(12), 5,7-tetraen-2-yl]-7-methoxy-1-methyl-benzimidazole-5-carbonyl]-3-piperidyl]carbamate (10 mg, 15.75 μmol, 45.63% yield) as a yellow solid. LC/MS (ESI⁺) [(M+H)⁺]: 634.8.

### Step 11:

To a solution of tert-butyl N-[(3R,5R)-5-fluoro-1-[2-[9-(3-hydroxypropyl)-11-methyl-1,9-diazatricyclo[6.3.1.04,12]dodeca-2,4(12), 5,7-tetraen-2-yl]-7-methoxy-1-methyl-benzimidazole-5-carbonyl]-3-piperidyl]carbamate (10 mg, 15.75 μmol) in DCM (1 mL), and added 4M HCl(dioxane) (1 mL). The mixture was stirred at 25° C. for 3 h, after completion of the reaction as judged by LC/MS. The mixture was concentrated under reduced pressure. The pH of the reaction mixture was adjusted to 8 with saturated Na₂CO₃ solution. The mixture was extracted with DCM (30 mL*3). The organic layer was washed with brine (10 mL) and dried over anhydrous sodium sulfate. The combined organic layer was concentrated in vacuo, and the residue was purified by silica gel flash column chromatography (eluting with DCM/MeOH=1:0-15:1) to give [(3R,5R)-3-amino-5-fluoro-1-piperidyl]-[2-[9-(3-hydroxypropyl)-11-methyl-1,9-diazatricyclo[6.3.1.04,12]dodeca-2,4(12), 5,7-tetraen-2-yl]-7-methoxy-1-methyl-benzimidazol-5-yl]methanone (3.1 mg, 5.80 μmol, 36.81% yield) as a white solid, LC/MS (ESI⁺) [(M+H)⁺]: 534.8. ¹H NMR (400 MHz, DMSO-d₆) δ 7.31 (s, 1H), 6.99 (s, 1H), 6.96-6.92 (m, 2H), 6.85 (s, 1H), 6.43 (dd, J=5.8, 2.5 Hz, 1H), 5.37 (d, J=24.7 Hz, 1H), 4.54 (t, J=5.0 Hz, 1H), 4.22 (d, J=6.5 Hz, 3H), 3.99 (s, 3H), 3.58 (d, J=11.3 Hz, 3H), 3.46 (dd, J=14.3, 6.8 Hz, 2H), 3.38 (d, J=12.0 Hz, 3H), 3.07 (s, 3H), 2.90 (s, 1H), 2.20 (s, 2H), 2.05-1.97 (m, 1H), 1.93-1.74 (m, 3H), 1.59 (d, J=42.8 Hz, 2H), 1.33 (d, J=14.7 Hz, 1H), 0.86 (d, J=7.0 Hz, 1H).

### Example 65 Preparation of ((R)-3-aminopiperidin-1-yl)(7-methoxy-1-methyl-2-(3-methyl-2,3-dihydro-1H-pyrrolo[1,2,3-de]quinoxalin-5-yl)-1H-benzo[d]imidazol-5-yl)methanone

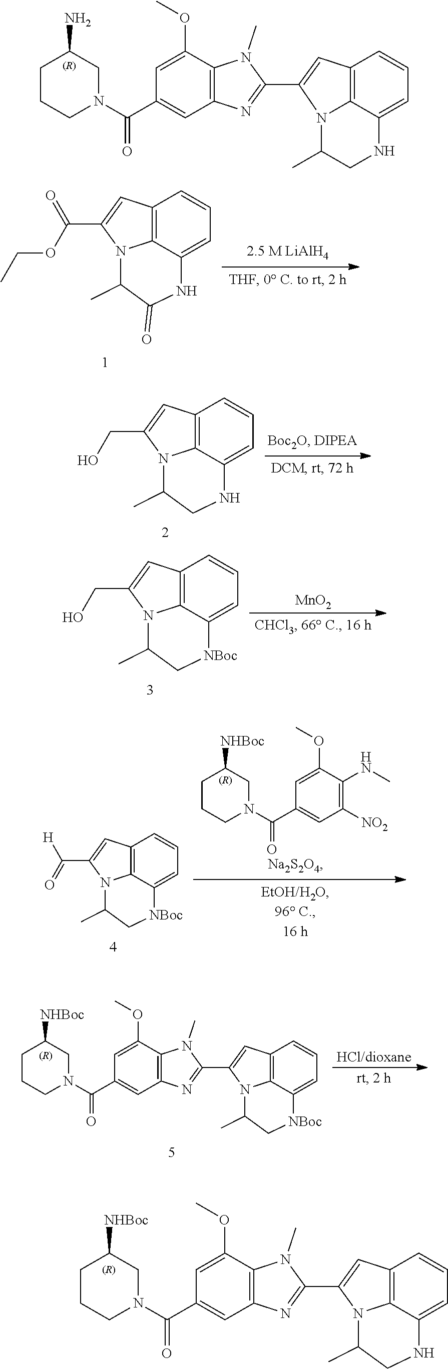

### Step 1:

To a solution of ethyl 11-methyl-10-oxo-1,9-diazatricyclo[6.3.1.0^{4,12}]dodeca-2,4(12), 5,7-tetraene-2-carboxylate (25 mg, 96.80 μmol) in anhydrous THF (5 mL) was added LiAlH₄ (2.5 M, 154.88 μL) at 0° C. The reaction mixture was stirred at RT for 2 h, quenched with excess EA (20 mL), stirred at rt for 15 minutes and filtered. The filtrate was concentrated in vacuo to afford crude (11-methyl-1,9-diazatricyclo[6.3.1.0^{4,12}]dodeca-2,4(12), 5,7-tetraen-2-yl)methanol (20 mg, 98.89 μmol, 51.08% yield). LC/MS (ESI⁺) [(M+H)⁺]: 203.

### Step 2:

To a solution of (11-methyl-1,9-diazatricyclo[6.3.1.0^{4,12}]dodeca-2,4(12), 5,7-tetraen-2-yl)methanol (20 mg, 98.89 μmol) in DCM was added tert-butoxycarbonyl tert-butyl carbonate (2.16 g, 9.89 mmol, 2.27 mL) and N-ethyl-N-isopropyl-propan-2-amine (2.56 g, 19.78 mmol, 3.44 mL). The reaction mixture was stirred at rt for 72 h, during which more DIPEA and Boc₂O was added until the conversion was completed. The reaction mixture was purified by flash column chromatography on silica gel to afford tert-butyl 2-(hydroxymethyl)-11-methyl-1,9-diazatricyclo[6.3.1.0^{4,12}]dodeca-2,4(12), 5,7-tetraene-9-carboxylate (10 mg, 33.07 μmol, 33.44% yield) was a white solid. LC/MS (ESI⁺) [(M+H)⁺]: 303.

### Step 3:

The mixture of tert-butyl 2-(hydroxymethyl)-11-methyl-1,9-diazatricyclo[6.3.1.0^{4,12}]dodeca-2,4(12), 5,7-tetraene-9-carboxylate (10 mg, 33.07 μmol) and MnO₂ (330.72 μmol) in chloroform, (2.5 mL) was stirred at 66° C. for 16 h. After cooling to RT, the reaction mixture was filtered. The filtrate was concentrated in vacuo to afford crude tert-butyl 2-formyl-11-methyl-1,9-diazatricyclo[6.3.1.0^{4,12}]dodeca-2,4(12), 5,7-tetraene-9-carboxylate (6 mg, 19.98 μmol, 60.40% yield). LC/MS (ESI⁺) [(M+H)⁺]: 301.

### Step 4:

A mixture of tert-butyl 2-formyl-11-methyl-1,9-diazatricyclo[6.3.1.0^{4,12}]dodeca-2,4(12), 5,7-tetraene-9-carboxylate (6 mg, 19.98 μmol), tert-butyl N-[(3R)-1-[3-methoxy-4-(methylamino)-5-nitro-benzoyl]-3-piperidyl]carbamate (10 mg, 24.48 μmol) and sodium dithionite (13.9 mg, 79.91 μmol) in mixed solvent of EtOH (5 mL) and H₂O (5 mL) was stirred at 96° C. for 16 h, cooled to RT and concentrated in vacuo. The residue was extracted with DCM (10 mL*3), and the organic phase was dried over anhydrous sodium sulfate and filtered. The filtrate was concentrated in vacuo and the residue was purified by prep-TLC (DCM/MeOH: 10/1) to afford tert-butyl 2-[5-[(3R)-3-(tert-butoxycarbonylamino)piperidine-1-carbonyl]-7-methoxy-1-methyl-benzimidazol-2-yl]-11-methyl-1,9-diazatricyclo[6.3.1.0⁴′]dodeca-2,4(12), 5,7-tetraene-9-carboxylate (2 mg, 3.04 μmol, 15.20% yield) as a white solid. LC/MS (ESI⁺) [(M+H)⁺]: 659.

### Step 5:

To a solution of mixture of tert-butyl 2-[5-[(3R)-3-(tert-butoxycarbonylamino)piperidine-1-carbonyl]-7-methoxy-1-methyl-benzimidazol-2-yl]-11-methyl-1,9-diazatricyclo[6.3.1.0^{4,12}]dodeca-2,4(12), 5,7-tetraene-9-carboxylate (5 mg, 7.59 μmol) in in dioxane (2 mL) was added 4.0 M hydrogen chloride solution. The resulting mixture was stirred at RT for 2 h and concentrated in vacuo to provide crude [(3R)-3-amino-1-piperidyl]-[7-methoxy-1-methyl-2-(11-methyl-1,9-diazatricyclo[6.3.1.0^{4,12}]dodeca-2,4(12), 5,7-tetraen-2-yl)benzimidazol-5-yl]methanone (3 mg, 6.54 μmol, 86.20% yield) as a yellow solid. LC/MS (ESI⁺) [(M+H)⁺]: 459.

### Example 66 Synthesis report of ((3R,5R)-3-amino-5-fluoropiperidin-1-yl)(1-ethyl-2-(1-(3-hydroxypropyl)-3-methyl-2,3-dihydro-1H-pyrrolo[1,2,3-de]quinoxalin-5-yl)-7-methoxy-1H-benzo[d]imidazol-5-yl)methanone

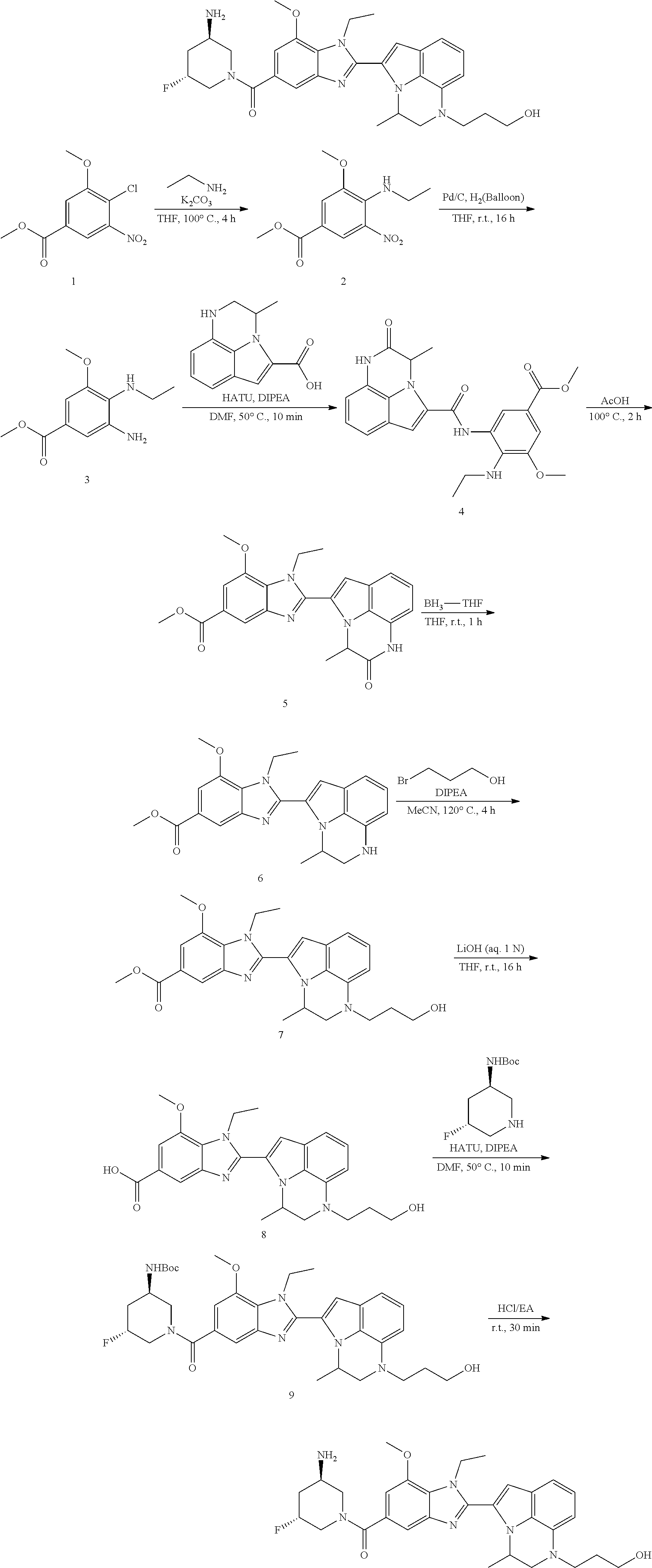

### Step 1:

To a solution of methyl 4-chloro-3-methoxy-5-nitro-benzoate (1.0 g, 4.07 mmol), ethanamine (183.55 mg, 4.07 mmol, 228.58 μL) in THF (10 mL) was added and potassium carbonate (1.69 g, 12.21 mmol, 737.17 μL). The resulting mixture was dissolved in THF (10 mL), stirred at 100° C. for 4 h. After cooling the mixture to rt, the reaction mixture was concentrated in vacuo and partitioned between EA (100 mL) and water (10 mL). The organic layer was washed further with water (2×10 ml) and saturated aqueous sodium chloride (10 mL). The organic layer was dried over sodium sulfate, filtered, and concentrated in vacuo to afford an orange powder which was purified by flash column chromatography on silica gel (eluting with EA/PE from 0 to 50%) to obtain methyl 4-(ethylamino)-3-methoxy-5-nitro-benzoate (950 mg, 3.74 mmol, 86.95% yield) as a yellow solid. LC/MS(ESI⁺)[(M+H)⁺]: 254.8.

### Step 2:

A mixture of methyl 4-(ethylamino)-3-methoxy-5-nitro-benzoate (950 mg, 3.74 mmol) and palladium 10% on carbon (79.5 mg, 747.33 μmol) in THF (20 mL) was hydrogenated in H₂ astmosphere (balloon) at ambient temperature for overnight. The mixture was filtered over Celite to remove Pd/C, and the solvent was evaporated to give the desired product methyl 3-amino-4-(ethylamino)-5-methoxy-benzoate (800 mg, 3.57 mmol, 95.47% yield) as a colorless solid. LC/MS(ESI⁺) [(M+H)⁺]: 224.8.

### Step 3:

A mixture of 11-methyl-10-oxo-1,9-diazatricyclo[6.3.1.04,12]dodeca-2,4,6,8(12)-tetraene-2-carboxylic acid (55 mg, 238.90 μmol), methyl 3-amino-4-(ethylamino)-5-methoxy-benzoate (53.6 mg, 238.90 μmol), HATU (90.9 mg, 238.90 μmol) and DIPEA (92.6 mg, 716.71 μmol, 124.83 μL) was dissolved in DMF (5 mL). The resulting mixture was stirred at 50° C. for 10 min, then diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give methyl 4-(ethylamino)-3-methoxy-5-[(11-methyl-10-oxo-1,9-diazatricyclo[6.3.1.04,12]dodeca-2,4,6,8(12)-tetraene-2-carbonyl)amino]benzoate (80 mg, 183.29 μmol, 76.72% yield) as yellow oil. LC/MS(ESI⁺) [(M+H)⁺]: 436.8.

### Step 4:

Methyl 4-(ethylamino)-3-methoxy-5-[(11-methyl-10-oxo-1,9-diazatricyclo[6.3.1.04,12]dodeca-2,4,6,8(12)-tetraene-2-carbonyl)amino]benzoate (80 mg, 183.29 μmol) was dissolved in acetic acid (5 mL) and the reaction mixture was stirred at 100° C. for 2 h. After cooling the reaction to RT, the solvent was removed in vacuo. The residue was purified by flash column chromatography on silica gel (eluting with EA/PE from 0 to 50%) to give methyl 1-ethyl-7-methoxy-2-(11-methyl-10-oxo-1,9-diazatricyclo[6.3.1.04,12]dodeca-2,4(12), 5,7-tetraen-2-yl)benzimidazole-5-carboxylate (60 mg, 143.39 μmol, 78.23% yield) as a white solid. LC/MS(ESI⁺) [(M+H)⁺]: 418.7.

### Step 5:

To a solution of methyl 1-ethyl-7-methoxy-2-(11-methyl-10-oxo-1,9-diazatricyclo[6.3.1.04,12]dodeca-2,4(12), 5,7-tetraen-2-yl)benzimidazole-5-carboxylate (60 mg, 143.39 μmol) in anhydrous THF (2 mL) was added Borane-tetrahydrofuran complex (0.5 mL) at 0° C. slowly. The reaction mixture was stirred at RT for 1 h, quenched with MeOH at 0° C. and concentrated in vacuo. The residue was diluted with 1 N HCl aqueous solution (1 mL), stirred at rt for 1 h, and basified with 1 N NaOH aqueous solution to pH 8. The resulting mixture was extracted with DCM (10 mL*3), and the combined organic phase was dried over anhydrous sodium sulfate and filtered. The filtrate was concentrated in vacuo and the residue was purified by silica gel flash column chromatography (eluting with DCM/MeOH=1:0-20:1) to give methyl 1-ethyl-7-methoxy-2-(11-methyl-1,9-diazatricyclo[6.3.1.04,12]dodeca-2,4(12), 5,7-tetraen-2-yl)benzimidazole-5-carboxylate (30 mg, 74.17 μmol, 51.73% yield) as a yellow solid. LC/MS(ESI⁺) [(M+H)⁺]: 404.8.

### Step 6:

A mixture of methyl 1-ethyl-7-methoxy-2-(11-methyl-1,9-diazatricyclo[6.3.1.04,12]dodeca-2,4(12), 5,7-tetraen-2-yl)benzimidazole-5-carboxylate (30 mg, 74.17 μmol), 3-bromopropan-1-ol (51.6 mg, 370.86 μmol, 32.42 μL) and DIPEA (95.86 mg, 741.73 μmol, 129.19 μL) was dissolved in Acetonitrile (3 mL). The resulting mixture was stirred at 120° C. for 4 h in microwave reactor. After removal of the solvent in vacuo. The residue was purified by flash column chromatography on silica gel (eluting with EA/PE from 0 to 100) to give methyl 1-ethyl-2-[9-(3-hydroxypropyl)-11-methyl-1,9-diazatricyclo[6.3.1.04,12]dodeca-2,4(12), 5,7-tetraen-2-yl]-7-methoxy-benzimidazole-5-carboxylate (20 mg, 43.24 μmol, 58.30% yield) as liquid oil. LC/MS(ESI⁺)[(M+H)⁺]: 462.8.

### Step 7:

To a solution of methyl 1-ethyl-2-[9-(3-hydroxypropyl)-11-methyl-1,9-diazatricyclo[6.3.1.04,12]dodeca-2,4(12), 5,7-tetraen-2-yl]-7-methoxy-benzimidazole-5-carboxylate (20 mg, 43.24 μmol) was dissolved in THF (1 mL) was added LiOH (aq. 1 N, 2 mL). The resulting mixture was stirred at rt for overnight. The pH was adjusted to be acidic with 2 mol/L HCl. After removal of the solvent in vacuo, the crude product 1-ethyl-2-[9-(3-hydroxypropyl)-11-methyl-1,9-diazatricyclo[6.3.1.04,12]dodeca-2,4(12), 5,7-tetraen-2-yl]-7-methoxy-benzimidazole-5-carboxylic acid (15 mg, 33.44 μmol, 77.35% yield) was obtained as a yellow solid. The crude product was used in next reaction without further purification. LC/MS(ESI⁺) [(M+H)⁺]: 448.8.

### Step 8:

A mixture of 1-ethyl-2-[9-(3-hydroxypropyl)-11-methyl-1,9-diazatricyclo[6.3.1.04,12]dodeca-2,4(12), 5,7-tetraen-2-yl]-7-methoxy-benzimidazole-5-carboxylic acid (15 mg, 33.44 μmol), tert-butyl N-[(3R,5R)-5-fluoro-3-piperidyl]carbamate (7.3 mg, 33.44 μmol), HATU (12.7 mg, 33.44 μmol) and DIPEA (13.0 mg, 100.33 μmol, 17.48 μL) was dissolved in DMF (3 mL). The mixture was stirred at 50° C. for 10 min, diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give tert-butyl N-[(3R,5R)-1-[1-ethyl-2-[9-(3-hydroxypropyl)-11-methyl-1,9-diazatricyclo[6.3.1.04,12]dodeca-2,4(12), 5,7-tetraen-2-yl]-7-methoxy-benzimidazole-5-carbonyl]-5-fluoro-3-piperidyl]carbamate (10 mg, 15.41 μmol, 46.09% yield) as yellow oil. LC/MS(ESI⁺) [(M+H)⁺]: 648.7.

### Step 9:

Tert-butyl N-[(3R,5R)-1-[1-ethyl-2-[9-(3-hydroxypropyl)-11-methyl-1,9-diazatricyclo[6.3.1.04,12]dodeca-2,4(12), 5,7-tetraen-2-yl]-7-methoxy-benzimidazole-5-carbonyl]-5-fluoro-3-piperidyl]carbamate (8 mg, 12.33 μmol) was dissolved in HCl/EA (2 mL). The resulting mixture was stirred at RT for 30 min. After removal the solvent in vacuo, the residue was purified by pre-HPLC to afford[(3R,5R)-3-amino-5-fluoro-1-piperidyl]-[1-ethyl-2-[9-(3-hydroxypropyl)-11-methyl-1,9-diazatricyclo[6.3.1.04,12]dodeca-2,4(12), 5,7-tetraen-2-yl]-7-methoxy-benzimidazol-5-yl]methanone (4 mg, 7.29 μmol, 59.12% yield) as white solid. LC/MS(ESI⁺) [(M+H)⁺]: 548.8. ¹H NMR (400 MHz, DMSO-d₆) δ 8.32 (s, 2H), 7.43 (s, 1H), 7.00 (d, J=6.2 Hz, 2H), 6.96 (s, 2H), 6.49 (dd, J=6.3, 2.2 Hz, 1H), 5.35 (s, 1H), 4.69-4.65 (m, 2H), 4.07 (s, 3H), 3.62 (dd, J=6.2, 2.4 Hz, 5H), 3.51 (d, J=7.0 Hz, 7H), 2.73 (d, J=2.0 Hz, 1H), 2.07 (d, J=7.8 Hz, 2H), 1.87 (d, J=7.2 Hz, 2H), 1.48 (t, J=7.1 Hz, 3H), 1.27 (dd, J=6.5, 2.3 Hz, 3H).

### The Following Compounds were Prepared Analogously

### Example 67 Synthesis Report of tert-butyl ((1R,4R,7R)-2-(1-ethyl-2-(1-(3-hydroxypropyl)-3-methyl-2,3-dihydro-1H-pyrrolo[1,2,3-de]quinoxalin-5-yl)-7-methoxy-1H-benzo[d]imidazole-5-carbonyl)-2-azabicyclo[2.2.1]heptan-7-yl)carbamate

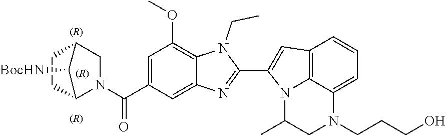

Prepared in analogous manner as for Example 66. LC/MS(ESI⁺) [(M+H)⁺]: 642.8. H NMR (400 MHz, DMSO-d₆) δ 7.48 (s, 1H), 7.36 (s, 1H), 7.03 (s, 1H), 6.96 (s, 2H), 6.90 (d, J=2.5 Hz, 1H), 6.43 (dd, J=6.4, 1.9 Hz, 1H), 4.60 (d, J=8.5 Hz, 2H), 4.53 (d, J=5.1 Hz, 1H), 4.17 (d, J=9.4 Hz, 1H), 4.03 (s, 3H), 3.70 (s, 1H), 3.56 (d, J=4.7 Hz, 3H), 3.49-3.44 (m, 2H), 3.27-3.24 (m, 2H), 2.68 (s, 1H), 2.03-1.92 (m, 2H), 1.81 (dt, J=13.8, 6.9 Hz, 4H), 1.51-1.30 (m, 14H), 1.21 (dd, J=15.0, 6.6 Hz, 3H).

### Example 68 Synthesis report of ((1R,4R,7R)-7-amino-2-azabicyclo[2.2.1]heptan-2-yl)(1-ethyl-2-(1-(3-hydroxypropyl)-3-methyl-2,3-dihydro-1H-pyrrolo[1,2,3-de]quinoxalin-5-yl)-7-methoxy-1H-benzo[d]imidazol-5-yl)methanone

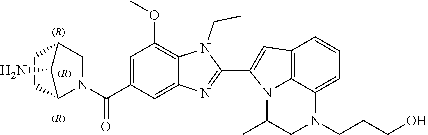

Prepared in analogous manner as for Example 66. LC/MS(ESI⁺) [(M+H)⁺]: 542.8. H NMR (400 MHz, DMSO-d₆) δ 8.22 (s, 1H), 7.36 (dd, J=2.6, 1.1 Hz, 1H), 6.98-6.93 (m, 3H), 6.90 (d, J=1.4 Hz, 1H), 6.44 (dd, J=6.3, 2.0 Hz, 1H), 5.35-5.28 (m, 1H), 4.60 (d, J=7.7 Hz, 2H), 4.02 (d, J=2.0 Hz, 3H), 3.80 (d, J=12.1 Hz, 1H), 3.56 (dt, J=7.2, 3.5 Hz, 3H), 3.47-3.43 (m, 2H), 3.39 (s, 1H), 3.19 (s, 1H), 3.08 (d, J=11.1 Hz, 1H), 2.68 (s, 1H), 2.22 (s, 1H), 1.97 (d, J=12.9 Hz, 2H), 1.90-1.72 (m, 4H), 1.42 (dt, J=8.4, 4.2 Hz, 4H), 1.21 (dd, J=9.1, 6.3 Hz, 3H).

### Example 69 Synthesis report of ((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-7-fluoro-1-methyl-1H-benzo[d]imidazol-5-yl)methanone

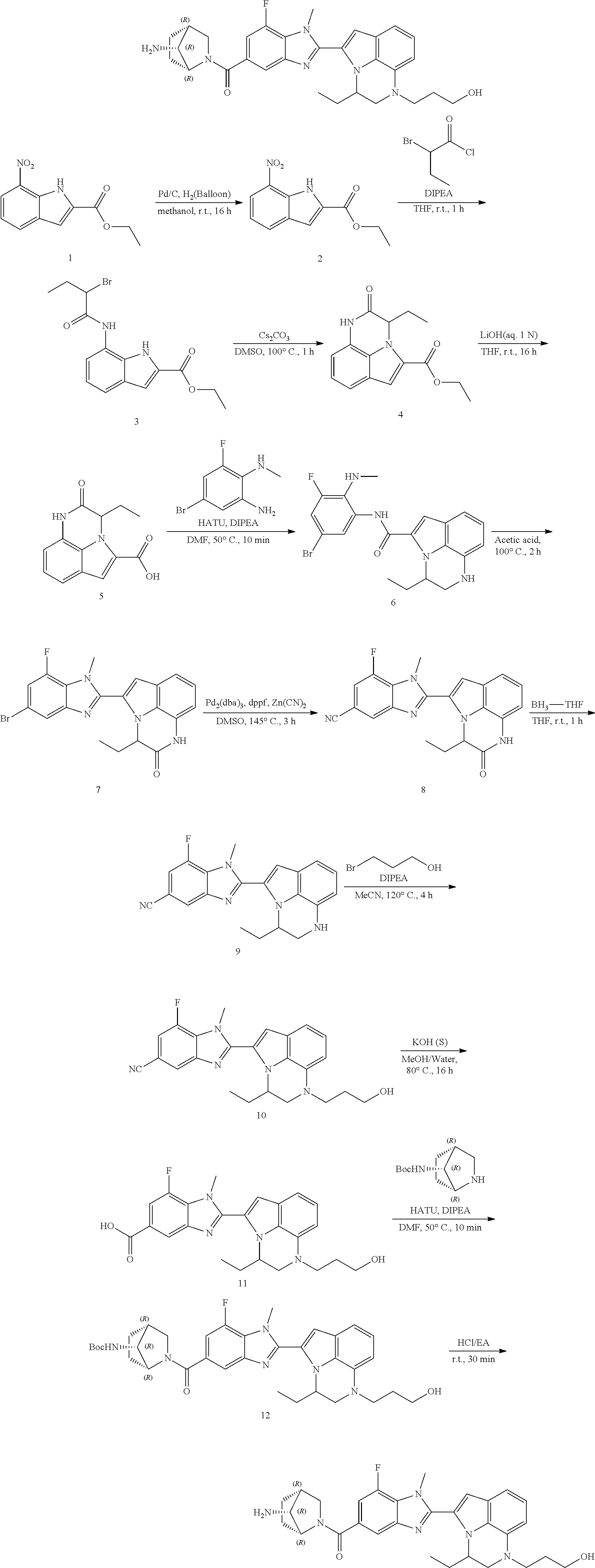

### Step 1:

A mixture of ethyl 7-nitro-1H-indole-2-carboxylate (7.0 g, 29.89 mmol) and palladium 10% on carbon (636.0 mg, 6.00 mmol) in methanol (200 mL) was hydrogenated in H₂ atomospher (balloon) at ambient temperature for overnight. The mixture was filtered over celite to remove Pd/C, and the solvent was evaporated to give the desired product ethyl 7-amino-1H-indole-2-carboxylate (6.0 g, 29.38 mmol, 98.30% yield) as a colorless solid. LC/MS(ESI⁺) [(M+H)⁺]: 204.8.

### Step 2:

A mixture of ethyl 7-amino-1H-indole-2-carboxylate (6 g, 29.38 mmol) and DIPEA (11.39 g, 88.14 mmol, 15.35 mL) was dissolved in THF (100 mL). The mixture was stirred at 0° C. and 2-bromobutanoyl chloride (5.45 g, 29.38 mmol) was added into the mixture with dropwise. The mixture was warmed to RT, stirred for 1 hour and diluted with water. The aqueous layer was extracted with ethyl acetate. The extract was washed with brine, dried over magnesium sulfate, and concentrated under vacuum. The residue was purified by flash column chromatography on silica gel (eluting with EA/PE from 0 to 50%) to give ethyl 7-(2-bromobutanoylamino)-1H-indole-2-carboxylate (9.0 g, 25.48 mmol, 86.73% yield) as white solid. LC/MS(ESI⁺) [(M+H)⁺]: 354.7.

### Step 3:

A suspension solution of ethyl 7-(2-bromobutanoylamino)-1H-indole-2-carboxylate (3 g, 8.49 mmol) and cesium carbonate (8.30 g, 25.48 mmol) in DMSO (15 mL) was stirred at 100° C. for 1 hour in sealed tube. After coolingto RT, the reaction mixture was diluted with ethyl acetate (20 mL), washed with water (10 mL). The organic phase was separated, dried over anhydrous sodium sulfate, and filtered. The filtrate was concentrated in vacuo, and the residue was purified by flash column chromatography on silica gel eluting with (EA:PE from 0 to 50%) to give ethyl 11-ethyl-10-oxo-1,9-diazatricyclo[6.3.1.04,12]dodeca-2,4,6,8(12)-tetraene-2-carboxylate (2 g, 7.34 mmol, 86.48% yield) as a white solid. LC/MS(ESI⁺) [(M+H)⁺]: 272.8.

### Step 4:

To a solutionl of Ethyl 11-ethyl-10-oxo-1,9-diazatricyclo[6.3.1.04,12]dodeca-2,4,6,8(12)-tetraene-2-carboxylate (3.0 g, 11.02 mmol) in THF (5 mL) was added LiOH (aq. 1 N, 10 mL). The resulting mixture was stirred at RT for overnight. The pH of the solution was adjusted to be acidic with 2 mol/L HCl. The resulting mixture was filtered and the obtained solid was dried in vacuo to give the product 11-ethyl-10-oxo-1,9-diazatricyclo[6.3.1.04,12]dodeca-2,4,6,8(12)-tetraene-2-carboxylic acid (2.3 g, 9.42 mmol, 85.47% yield) as a yellow solid. The crude product was used in next step reaction without further purification. LC/MS(ESI⁺) [(M+H)⁺]: 244.8.

### Step 5:

A mixture of 11-ethyl-10-oxo-1,9-diazatricyclo[6.3.1.04,12]dodeca-2,4,6,8(12)-tetraene-2-carboxylic acid (200 mg, 818.85 μmol), 5-bromo-3-fluoro-N2-methyl-benzene-1,2-diamine (179.4 mg, 818.85 μmol), HATU (311.4 mg, 818.85 μmol) and DIPEA (317.5 mg, 2.46 mmol, 427.88 μL) was dissolved in DMF (5 mL). The resulting mixture was stirred at 50° C. for 10 min. The reaction mixture was diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give N-[5-bromo-3-fluoro-2-(methylamino)phenyl]-11-ethyl-10-oxo-1,9-diazatricyclo[6.3.1.04,12]dodeca-2,4,6,8(12)-tetraene-2-carboxamide (150 mg, 336.86 μmol, 41.14% yield) as a yellow solid. LC/MS(ESI⁺) [(M+H)⁺]: 446.6.

### Step 6:

N-[5-bromo-3-fluoro-2-(methylamino)phenyl]-11-ethyl-10-oxo-1,9-diazatricyclo [6.3.1.04,12]dodeca-2,4,6,8(12)-tetraene-2-carboxamide (150 mg, 336.86 μmol) was dissolved in acetic acid (10 mL) and stirred at 100° C. for 2 h. The reaction was allowed to cool to RT, after removal of the solvent in vacuo, the residue was purified by flash column chromatography on silica gel (eluting with EA/PE from 0 to 50%) to give 2-(5-bromo-7-fluoro-1-methyl-benzimidazol-2-yl)-11-ethyl-1,9-diazatricyclo[6.3.1.04,12]dodeca-2,4(12), 5,7-tetraen-10-one (100 mg, 234.04 μmol, 69.48% yield) as a white solid. LC/MS(ESI⁺) [(M+H)⁺]: 428.6.

### Step 7:

A mixture of 2-(5-bromo-7-fluoro-1-methyl-benzimidazol-2-yl)-11-ethyl-1,9-diazatricyclo[6.3.1.04,12]dodeca-2,4(12), 5,7-tetraen-10-one (200 mg, 468.09 μmol), dicyanozinc (54.97 mg, 468.09 μmol, 29.68 μL), Tris(Dibenzylideneacetone)dipalladium (0) (428.64 mg, 468.09 μmol) and 1,1′-Bis(diphenylphosphino)ferrocene (259.50 mg, 468.09 μmol) was dissolved in DMSO (5 mL). The reaction mixture was heated at 145° C. under the atmosphere of Nitrogen for 3 h in microwave reactor. The mixture was purified by flash column chromatography on silica gel (eluting with PE/EA from 0 to 50%) to give 2-(11-ethyl-10-oxo-1,9-diazatricyclo[6.3.1.04,12]dodeca-2,4(12), 5,7-tetraen-2-yl)-7-fluoro-1-methyl-benzimidazole-5-carbonitrile (150 mg, 401.73 μmol, 85.82% yield) as yellow liquid oil. LC/MS[(M+H)⁺]: 373.8.

### Step 8:

To a solution of 2-(11-ethyl-10-oxo-1,9-diazatricyclo[6.3.1.04,12]dodeca-2,4(12), 5,7-tetraen-2-yl)-7-fluoro-1-methyl-benzimidazole-5-carbonitrile (150 mg, 401.73 μmol) in anhydrous THF (10 mL) was added borane-tetrahydrofuran complex (10 mL) at 0° C. slowly. The reaction mixture was stirred at rt for 1 h, quenched with MeOH at 0° C. and concentrated in vacuo. The residue was diluted with 1 N HCl aqueous solution (1 mL), stirred at rt for 1 h, and basified with 1 N NaOH aqueous solution to pH ˜8. The resulting mixture was extracted with DCM (10 mL*3), and the combined organic phase was dried over anhydrous sodium sulfate and filtered. The filtrate was concentrated in vacuo and the residue was purified by silica gel flash column chromatography (elution with DCM/MeOH=1:0-20:1) to give 2-(11-ethyl-1,9-diazatricyclo[6.3.1.04,12]dodeca-2,4(12), 5,7-tetraen-2-yl)-7-fluoro-1-methyl-benzimidazole-5-carbonitrile (100 mg, 278.24 μmol, 69.26% yield) as a yellow solid. LC/MS(ESI⁺) [(M+H)⁺]: 359.8.

### Step 9:

A mixture of 2-(11-ethyl-1,9-diazatricyclo[6.3.1.04,12]dodeca-2,4(12), 5,7-tetraen-2-yl)-7-fluoro-1-methyl-benzimidazole-5-carbonitrile (100 mg, 278.24 μmol), 3-bromopropan-1-ol (116.0 mg, 834.73 μmol, 72.97 μL) and DIPEA (179.8 mg, 1.39 mmol, 242.32 μL) was dissolved in acetonitrile (5 mL). The resulting mixture was stirred at 120° C. for 4 h with microwave reactor. After removal of the solvent in vacuo. The residue was purified by flash column chromatography on silica gel (eluting with EA/PE from 0 to 100) to give 2-[11-ethyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carbonitrile (80 mg, 191.63 μmol, 68.87% yield) as liquid oil. LC/MS(ESI⁺)[(M+H)⁺]: 417.8.

### Step 10:

A mixture of 2-[11-ethyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carbonitrile (80 mg, 191.63 μmol) and KOH (s) (32.8 mg, 575.00 μmol) was dissolved in methanol/water (5 mL). the resulting mixture was stirred at 80° C. for overnight. The desired signal was found by LC/MS. The mixture was acidified with 3 mol/L hydrochloric acid, extracted with DCM (10 mL*3), the organice phase was concentrated under reduced pressure, the residue was purified by pre-HPLC to give 2-[11-ethyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carboxylic acid (50 mg, 114.55 μmol, 59.78% yield) as a yellow solid. LC/MS(ESI⁺)[(M+H)⁺]: 436.8.

### Step 11:

A mixture of 2-[11-ethyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carboxylic acid (30 mg, 68.73 μmol), tert-butyl N-[(1R,4R,7R)-2-azabicyclo[2.2.1]heptan-7-yl]carbamate (PharmaBlock) (14.6 mg, 68.73 μmol), HATU (26.1 mg, 68.73 μmol) and DIPEA (26.7 mg, 206.20 μmol, 35.91 μL) was dissolved in DMF (5 mL). The resulting mixture was stirred at 50° C. for 10 min, diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give tert-butyl N-[(1R,4R,7R)-2-[2-[11-ethyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carbonyl]-2-azabicyclo[2.2.1]heptan-7-yl]carbamate (20 mg, 31.71 μmol, 46.13% yield) as yellow oil. LC/MS(ESI⁺) [(M+H)⁺]: 630.8.

### Step 12:

Tert-butyl N-[(1R,4R,7R)-2-[2-[11-ethyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carbonyl]-2-azabicyclo[2.2.1]heptan-7-yl]carbamate (10 mg, 15.85 μmol) was dissolved in HCl/EA (3 mL). The resulting mixture was stirred at RTfor 30 min. After removal of the solvent in vacuo, the residue was purified by pre-HPLC to afford [(1R,4R,7R)-7-amino-2-azabicyclo[2.2.1]heptan-2-yl]-[2-[11-ethyl-9-(3-hydroxypropyl)-1,9-diazatricyclo[6.3.1.04,12]dodeca-2,4(12), 5,7-tetraen-2-yl]-7-fluoro-1-methyl-benzimidazol-5-yl]methanone (5 mg, 9.42 μmol, 59.43% yield) as a white solid. LC/MS(ESI⁺) [(M+H)⁺]: 530.8. ¹H NMR (400 MHz, DMSO-d₆) δ 8.20 (s, 1H), 7.60 (d, J=4.1 Hz, 1H), 7.21 (d, J=11.9 Hz, 1H), 7.05 (d, J=1.3 Hz, 1H), 6.98-6.94 (m, 2H), 6.44 (dd, J=6.2, 2.1 Hz, 1H), 5.26 (s, 1H), 4.54 (s, 1H), 4.17 (d, J=3.2 Hz, 3H), 3.73 (d, J=11.7 Hz, 1H), 3.56 (t, J=2.9 Hz, 4H), 3.48-3.43 (m, 2H), 3.21 (s, 1H), 3.09 (d, J=11.0 Hz, 1H), 2.68 (s, 1H), 2.22 (s, 1H), 1.97 (s, 2H), 1.89-1.70 (m, 4H), 1.61 (dt, J=7.2, 3.7 Hz, 2H), 0.71 (td, J=7.4, 3.4 Hz, 3H).

### Example 70 ((1R,4R,7R)-7-amino-2-azabicyclo[2.2.1]heptan-2-yl)(2-((R)-3-ethyl-1-(3-hydroxypropyl)-2,3-dihydro-1H-pyrrolo[1,2,3-de]quinoxalin-5-yl)-7-fluoro-1-methyl-1H-benzo[d]imidazol-5-yl)methanone and Example 71 ((1R,4R,7R)-7-amino-2-azabicyclo[2.2.1]heptan-2-yl)(2-((S)-3-ethyl-1-(3-hydroxypropyl)-2,3-dihydro-1H-pyrrolo[1,2,3-de]quinoxalin-5-yl)-7-fluoro-1-methyl-1H-benzo[d]imidazol-5-yl)methanone

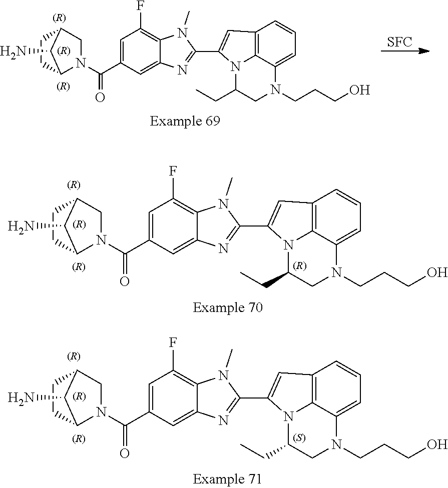

((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-7-fluoro-1-methyl-1H-benzo[d]imidazol-5-yl)methanone (360 mg, 0.68 mmol) was chirally separated by SFC with mobile phase (CO₂/(MeOH/ACN(0.2% Methanol Ammonia)=3:2)=45/55) (Column: oz 20*250 mm, 10 um (Daicel)) (Flow Rate: 110 g/min) to give synthetic Example 70 (110.0 mg, 30.6%) as an off-white solid (LCMS(ESI⁺) [(M+H)⁺]: 530.8) ¹H NMR (400 MHz, DMSO-d₆) δ 7.70-7.58 (m, 1H), 7.30-7.18 (m, 1H), 7.04 (d, J=3.3 Hz, 1H), 6.98-6.93 (m, 2H), 6.43 (dd, J=6.2, 2.2 Hz, 1H), 5.29-5.21 (m, 1H), 4.57 (t, J=5.1 Hz, 1H), 4.16 (d, J=3.7 Hz, 3H), 3.73 (d, J=2.3 Hz, 1H), 3.54 (q, J=4.3 Hz, 4H), 3.49-3.43 (m, 2H), 3.39 (s, 1H), 3.19 (s, 1H), 3.11-3.03 (m, 1H), 2.23-2.10 (m, 1H), 2.03-1.89 (m, 2H), 1.85-1.72 (m, 3H), 1.64-1.58 (m, 2H), 1.47-1.34 (m, 1H), 0.70 (t, J=7.4 Hz, 3H). and Example 71 (146.3 mg, 40.6%) as an off-white solid (LCMS(ESI⁺) [(M+H)⁺]: 530.8); ¹H NMR (400 MHz, DMSO-d₆) δ 7.59 (d, J=1.2 Hz, 1H), 7.20 (d, J=11.9 Hz, 1H), 7.04 (s, 1H), 6.95 (d, J=5.9 Hz, 2H), 6.45-6.42 (m, 1H), 4.57 (t, J=5.0 Hz, 1H), 4.17 (d, J=2.9 Hz, 3H), 3.71 (s, 1H), 3.55 (s, 4H), 3.48-3.43 (m, 2H), 3.20 (s, 2H), 3.08 (d, J=11.0 Hz, 1H), 2.22 (s, 1H), 2.14 (s, 1H), 1.95 (s, 2H), 1.80 (dd, J=14.8, 7.3 Hz, 2H), 1.60 (t, J=7.2 Hz, 2H), 1.24 (s, 2H), 0.69 (t, J=7.4 Hz, 3H).

The Examples 70 and 71 were tested in PAD 4 biochemical assay. The more potent Example 71 was used for co-cystallization with the PAD4 protein and structural determination. The co-cystallization procedure is described in Biologic Exampl 6. The crystal structure of Example 71 and the PAD4 protein determined that the ethyl group on the 2,3-dihydro-1H-pyrrolo[1,2,3-de]quinoxalin-5-yl) ring of Example 71 has a S configuration. It was concluded that the S isomer of other structurally similar compounds is more potent than the corresponding R isomer.

### The Following Compounds were Prepared Analogously

### Example 72 Preparation of ((1R,4R,7R)-7-amino-2-azabicyclo[2.2.1]heptan-2-yl)(2-(3-ethyl-2,3-dihydro-1H-pyrrolo[1,2,3-de]quinoxalin-5-yl)-7-fluoro-1-methyl-1H-benzo[d]imidazol-5-yl)methanone

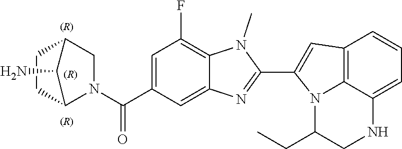

Prepared in analogous manner as for Example 69. LC-MS: (ESI⁺) m/z 472.7 [M+H]⁺. ¹H NMR (400 MHz, DMSO-d₆) δ 8.18 (s, 1H), 7.72-7.57 (m, 1H), 7.24 (dd, J=29.1, 12.1 Hz, 1H), 7.02 (s, 1H), 6.94 (d, J=7.9 Hz, 1H), 6.87 (t, J=7.6 Hz, 1H), 6.38 (d, J=7.1 Hz, 1H), 6.07 (s, 1H), 5.23 (s, 1H), 4.17 (s, 3H), 3.77 (d, J=12.3 Hz, 1H), 3.64 (s, 1H), 3.36 (d, J=5.8 Hz, 1H), 3.23 (s, 1H), 3.07 (dd, J=19.8, 9.9 Hz, 1H), 2.23 (d, J=23.6 Hz, 1H), 1.96 (s, 2H), 1.75 (t, J=8.8 Hz, 1H), 1.67-1.53 (m, 2H), 1.50-1.36 (m, 1H), 0.73-0.62 (m, 3H).

### Example 73 ((1R,4R,7R)-7-amino-2-azabicyclo[2.2.1]heptan-2-yl)(2-((R)-3-ethyl-2,3-dihydro-1H-pyrrolo[1,2,3-de]quinoxalin-5-yl)-7-fluoro-1-methyl-1H-benzo[d]imidazol-5-yl)methanone and

### Example 74 ((1R,4R,7R)-7-amino-2-azabicyclo[2.2.1]heptan-2-yl)(2-((S)-3-ethyl-2,3-dihydro-1H-pyrrolo[1,2,3-de]quinoxalin-5-yl)-7-fluoro-1-methyl-1H-benzo[d]imidazol-5-yl)methanone

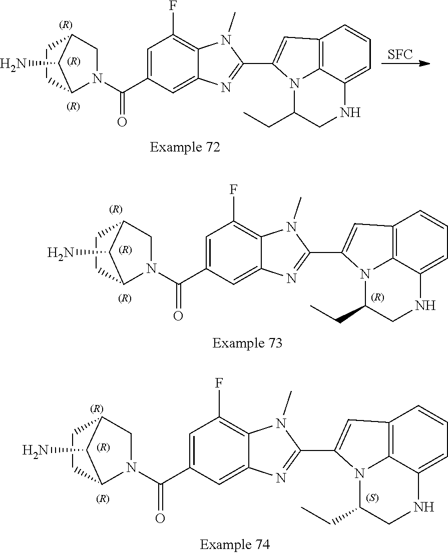

((1R,4R,7R)-7-amino-2-azabicyclo[2.2.1]heptan-2-yl)(2-(3-ethyl-2,3-dihydro-1H-pyrrolo[1,2,3-de]quinoxalin-5-yl)-7-fluoro-1-methyl-1H-benzo[d]imidazol-5-yl)methanone (26 mg, 0.55 mmol) was chirally separated by SFC with mobile phase (CO2/(MEOH:ACN(0.2% Methanol Ammonia)=1:1)=55/45) (Column: OZ 20*250 mm, 10 um (Daicel) (Flow Rate: 110 g/min) to give synthetic Example 73 (7 mg, 26.9%) as an off-white solid (LCMS(ESI⁺) [(M+H)⁺]: 472.7) and Example 74 (12 mg, 26.1%) as off-white solid (LCMS(ESI⁺) [(M+H)⁺]: 472.7).

Based on the co-cystallization results of Example 71, it is believe that Example 74, which is more potent than Example 73 in the PAD4 biochemical assay, has the S configuration.

### Example 75 Synthesis report of ((3R,5R)-3-amino-5-fluoropiperidin-1-yl)(2-(3-ethyl-1-(3-hydroxypropyl)-2,3-dihydro-1H-pyrrolo[1,2,3-de]quinoxalin-5-yl)-7-fluoro-1-methyl-1H-benzo[d]imidazol-5-yl)methanone

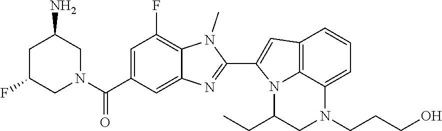 [SMILES: CCC1CN(CCCO)c2cccc3cc(-c4nc5cc(C(=O)N6C[C@H](N)C[C@@H](F)C6)cc(F)c5n4C)n1c23]

Prepared in analogous manner as for Example 69. LC/MS(ESI⁺) [(M+H)⁺]: 536.8. ¹H NMR (400 MHz, DMSO-d₆) δ 7.55 (d, J=1.2 Hz, 1H), 7.15 (d, J=11.9 Hz, 1H), 7.05 (s, 1H), 6.98-6.94 (m, 2H), 6.44 (dd, J=6.3, 2.1 Hz, 1H), 5.26 (s, 1H), 4.54 (t, J=5.0 Hz, 1H), 4.18 (s, 3H), 3.56 (d, J=6.1 Hz, 4H), 3.46 (q, J=7.3 Hz, 2H), 2.98 (s, 2H), 2.68 (s, 1H), 2.15 (s, 2H), 1.83 (dd, J=14.6, 7.4 Hz, 2H), 1.62 (d, J=7.7 Hz, 2H), 0.72 (t, J=7.4 Hz, 3H).

### Example 76 ((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-7-fluoro-1-methyl-6-(methylamino)-1H-benzo[d]imidazol-5-yl)methanone

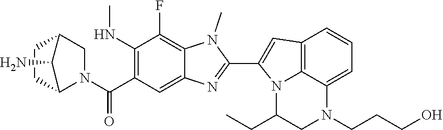 [SMILES: CCC1CN(CCCO)c2cccc3cc(-c4nc5cc(C(=O)N6C[C@H]7CC[C@@H]6[C@@H]7N)c(NC)c(F)c5n4C)n1c23]

Prepared in analogous manner as for Example 69. LC/MS(ESI⁺) [(M+H)⁺]: 559.8.

### Example 77 ((1R,4R,7R)-7-amino-2-azabicyclo[2.2.1]heptan-2-yl)(1-(cyclopropylmethyl)-2-(3-ethyl-1-(3-hydroxypropyl)-2,3-dihydro-1H-pyrrolo[1,2,3-de]quinoxalin-5-yl)-7-fluoro-1H-benzo[d]imidazol-5-yl)methanone

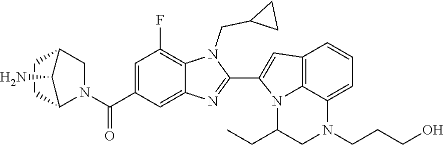 [SMILES: CCC1CN(CCCO)c2cccc3cc(-c4nc5cc(C(=O)N6C[C@H]7CC[C@@H]6[C@@H]7N)cc(F)c5n4CC4CC4)n1c23]

Prepared in analogous manner as for Example 69. LC/MS(ESI⁺) [(M+H)⁺]: 570.8.

### Example 78 ((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-6,7-difluoro-1-methyl-1H-benzo[d]imidazol-5-yl)methanone

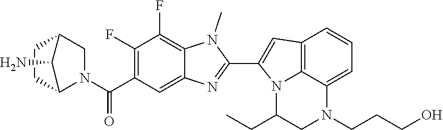 [SMILES: CCC1CN(CCCO)c2cccc3cc(-c4nc5cc(C(=O)N6C[C@H]7CC[C@@H]6[C@@H]7N)c(F)c(F)c5n4C)n1c23]

Prepared in analogous manner as for Example 69. LC/MS(ESI⁺) [(M+H)⁺]: 548.7.

### Example 79 Preparation of ((3R,5R)-3-amino-5-fluoropiperidin-1-yl)(2-(3-ethyl-1-(2-hydroxyethyl)-2,3-dihydro-1H-pyrrolo[1,2,3-de]quinoxalin-5-yl)-7-methoxy-1-methyl-1H-benzo[d]imidazol-5-yl)methanone

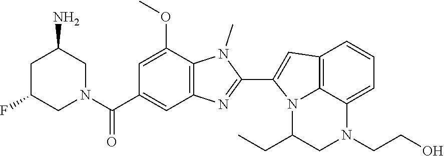 [SMILES: CCC1CN(CCO)c2cccc3cc(-c4nc5cc(C(=O)N6C[C@H](N)C[C@@H](F)C6)cc(OC)c5n4C)n1c23]

Prepared in analogous manner as for Example 69. LC/MS (ESI⁺) [(M+H)⁺]: 534.8. ¹H NMR (400 MHz, DMSO-d₆) δ 7.30 (s, 1H), 6.98 (d, J=2.8 Hz, 1H), 6.94 (t, J=4.7 Hz, 2H), 6.84 (s, 1H), 6.41 (s, 1H), 5.22 (s, 1H), 4.73-4.54 (m, 1H), 4.23 (d, J=16.6 Hz, 3H), 4.01 (d, J=15.7 Hz, 4H), 3.89-3.61 (m, 5H), 3.49 (d, J=6.1 Hz, 3H), 3.35 (s, 2H), 3.26 (d, J=5.7 Hz, 3H), 3.01 (s, 2H), 2.16 (s, 2H), 1.59 (s, 3H), 1.35 (s, 1H), 0.67 (t, J=6.5 Hz, 3H).

### Example 80 Preparation of ((3R,5R)-3-amino-5-fluoropiperidin-1-yl)(2-(3-ethyl-1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone

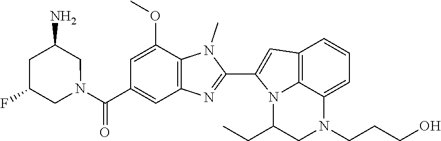 [SMILES: CCC1CN(CCCO)c2cccc3cc(-c4nc5cc(C(=O)N6C[C@H](N)C[C@@H](F)C6)cc(OC)c5n4C)n1c23]

Prepared in analogous manner as for Example 69. LC/MS (ESI⁺) [(M+H)⁺]: 548.8.

### Example 81 and Example 82

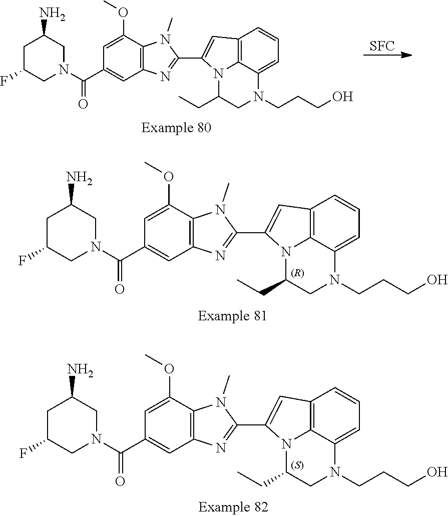

Example 80 [(3R,5R)-3-amino-5-fluoro-1-piperidyl]-[2-[11-ethyl-9-(3-hydroxypropyl)-1,9-diazatricyclo[6.3.1.04,12]dodeca-2,4(12), 5,7-tetraen-2-yl]-7-methoxy-1-methyl-benzimidazol-5-yl]methanone(12 mg) was separated by SFC with similar prep conditions in Examples 73 and 74 to obtain Example 81 (3.1 mg, 25.8%) and Example 82 (4.2 mg, 35.0%).

Based on the co-cystallization results of Example 71, it is believed that Example 82, which is more potent than Example 81 in the PAD4 biochemical assay, has the S configuration.

### Example 83 Preparation of ((3R,5R)-3-amino-5-fluoropiperidin-1-yl)(2-(3-ethyl-1-(3-hydroxy-3-methylbutyl)-2,3-dihydro-1H-pyrrolo[1,2,3-de]quinoxalin-5-yl)-7-methoxy-1-methyl-1H-benzo[d]imidazol-5-yl)methanon

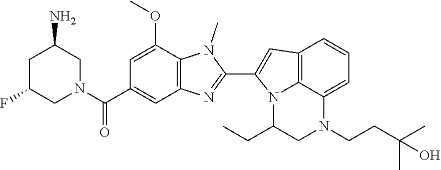 [SMILES: CCC1CN(CCC(C)(C)O)c2cccc3cc(-c4nc5cc(C(=O)N6C[C@H](N)C[C@@H](F)C6)cc(OC)c5n4C)n1c23]

Prepared in analogous manner as for Example 69. LC/MS (ESI⁺) [(M+H)⁺]: 576.8. ¹H NMR (400 MHz, DMSO-d₆) δ 7.40-7.31 (m, 1H), 6.99 (d, J=3.2 Hz, 1H), 6.95 (d, J=3.5 Hz, 2H), 6.87 (d, J=12.7 Hz, 1H), 6.42 (d, J=3.5 Hz, 1H), 5.24 (s, 1H), 4.37 (s, 1H), 4.22 (s, 3H), 3.99 (s, 3H), 3.52 (dd, J=26.4, 10.6 Hz, 5H), 3.35 (d, J=6.6 Hz, 2H), 2.25 (d, J=71.1 Hz, 2H), 1.77 (s, 3H), 1.60 (s, 2H), 1.29 (dd, J=29.2, 10.0 Hz, 2H), 1.21 (s, 6H), 0.68 (dd, J=8.3, 6.5 Hz, 3H).

### Example 84 Preparation of ((3R,5R)-3-amino-5-fluoropiperidin-1-yl)(2-(3-ethyl-1-(3-methoxypropyl)-2,3-dihydro-1H-pyrrolo[1,2,3-de]quinoxalin-5-yl)-7-methoxy-1-methyl-1H-benzo[d]imidazol-5-yl)methanone

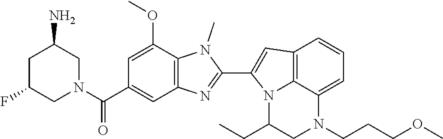 [SMILES: CCC1CN(CCCOC)c2cccc3cc(-c4nc5cc(C(=O)N6C[C@H](N)C[C@@H](F)C6)cc(OC)c5n4C)n1c23]

Prepared in analogous manner as for Example 69. LC/MS (ESI⁺) [(M+H)⁺]: 561.8. ¹H NMR (400 MHz, DMSO-d₆) δ 7.30 (s, 1H), 7.02-6.91 (m, 3H), 6.84 (s, 1H), 6.42-6.38 (m, 1H), 4.21 (s, 3H), 3.99 (s, 3H), 3.60-3.51 (m, 2H), 3.45 (dt, J=15.1, 7.5 Hz, 4H), 3.28 (s, 3H), 3.24 (d, J=15.3 Hz, 3H), 2.15 (s, 1H), 2.02 (d, J=7.6 Hz, 1H), 1.96-1.82 (m, 3H), 1.65-1.53 (m, 3H), 1.35 (t, J=15.0 Hz, 2H), 0.87 (s, 1H), 0.69 (dd, J=16.5, 9.1 Hz, 3H).

### Example 85 Preparation of ((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-7-methoxy-1-methyl-1H-benzo[d]imidazol-5-yl)methanone

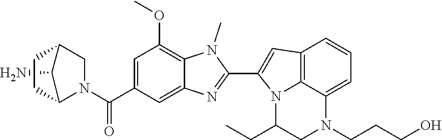 [SMILES: CCC1CN(CCCO)c2cccc3cc(-c4nc5cc(C(=O)N6C[C@H]7CC[C@@H]6[C@@H]7N)cc(OC)c5n4C)n1c23]

Prepared in analogous manner as for Example 69. LC/MS (ESI⁺) [(M+H)⁺]: 542.8.

### Example 86 and Example 87

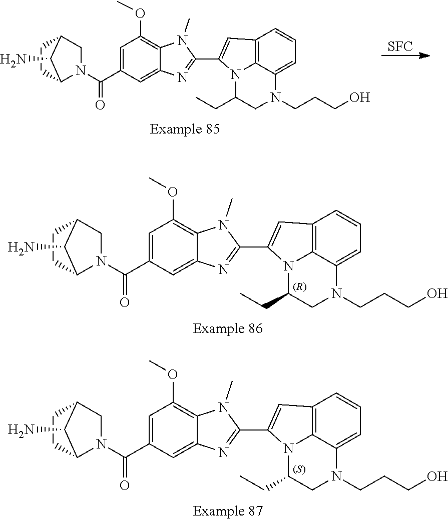

Example 85 [(1R,4R,7R)-7-amino-2-azabicyclo[2.2.1]heptan-2-yl]-[2-[11-ethyl-9-(3-hydroxypropyl)-1,9-diazatricyclo[6.3.1.04,12]dodeca-2,4(12), 5,7-tetraen-2-yl]-7-methoxy-1-methyl-benzimidazol-5-yl]methanone (61.1 mg) was separated by SFC with similar prep conditions in Examples 73 and 74 to obtain Example 86 (18 mg, 29.4%) and Example 87 (20 mg, 32.7%).

Based on the co-cystallization results of Example 71, it is believed that Example 87, which is more potent than Example 86 in the PAD4 biochemical assay, has the S configuration.

### Example 88 Preparation of ((1R,4R,7R)-7-amino-2-azabicyclo[2.2.1]heptan-2-yl)(2-(3-ethyl-1-(3-hydroxy-3-methylbutyl)-2,3-dihydro-1H-pyrrolo[1,2,3-de]quinoxalin-5-yl)-7-fluoro-1-methyl-1H-benzo[d]imidazol-5-yl)methanone

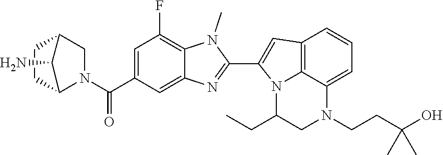 [SMILES: CCC1CN(CCC(C)(C)O)c2cccc3cc(-c4nc5cc(C(=O)N6C[C@H]7CC[C@@H]6[C@@H]7N)cc(F)c5n4C)n1c23]

Prepared in analogous manner as for Example 69. LC/MS (ESI⁺) [(M+H)⁺]: 558.8. H NMR (400 MHz, DMSO-d₆) δ 8.21 (s, 0.35H), 7.60 (d, J=4.3 Hz, 1H), 7.25 (dd, J=28.3, 12.1 Hz, 1H), 7.05 (s, 1H), 6.98-6.92 (m, 2H), 6.43 (dd, J=4.9, 3.3 Hz, 1H), 5.26 (s, 1H), 4.39 (s, 1H), 4.18 (s, 3H), 3.74 (d, J=12.4 Hz, 1H), 3.54 (d dd, J=11.8, 8.0, 3.2 Hz, 4H), 3.45 (dd, J=14.2, 5.4 Hz, 1H), 3.21 (s, 1H), 3.12-3.05 (m, 1H), 2.23 (s, 1H), 1.97 (s, 2H), 1.84-1.67 (m, 3H), 1.65-1.56 (m, 2H), 1.42 (dd, J=22.7, 10.4 Hz, 1H), 1.23 (d, J=15.7 Hz, 6H), 0.71 (td, J=7.4, 3.8 Hz, 3H).

### Example 89 Preparation of ((1R,4R,7R)-7-amino-2-azabicyclo[2.2.1]heptan-2-yl)(2-(3-ethyl-1-(3-hydroxy-2,2-dimethylpropyl)-2,3-dihydro-1H-pyrrolo[1,2,3-de]quinoxalin-5-yl)-7-fluoro-1-methyl-1H-benzo[d]imidazol-5-yl)methanone

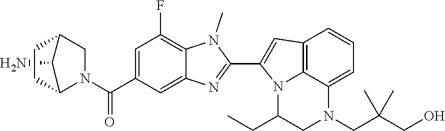 [SMILES: CCC1CN(CC(C)(C)CO)c2cccc3cc(-c4nc5cc(C(=O)N6C[C@H]7CC[C@@H]6[C@@H]7N)cc(F)c5n4C)n1c23]

Prepared in analogous manner as for Example 69. LC-MS (ESI⁺): m/z 558.7 [M+H]⁺.

### Example 90 Preparation of ((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-7-methoxy-1-(prop-2-yn-1-yl)-1H-benzo[d]imidazol-5-yl)methanone

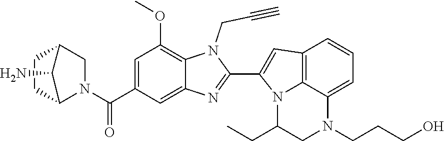 [SMILES: C#CCn1c(-c2cc3cccc4c3n2C(CC)CN4CCCO)nc2cc(C(=O)N3C[C@H]4CC[C@@H]3[C@@H]4N)cc(OC)c21]

Prepared in analogous manner as for Example 69. LC-MS: (ESI⁺) m/z 566.8 [M+H]⁺. ¹H NMR (400 MHz, DMSO-d₆) δ 8.22 (s, 0.45H), 7.37 (d, J=3.9 Hz, 1H), 7.09 (s, 1H), 7.01-6.91 (m, 3H), 6.43 (d, J=6.6 Hz, 1H), 5.49 (d, J=17.9 Hz, 1H), 5.33 (d, J=18.2 Hz, 1H), 5.24 (s, 1H), 4.01 (s, 3H), 3.79 (d, J=11.6 Hz, 1H), 3.54 (d, J=3.9 Hz, 3H), 3.51 (d, J=2.1 Hz, 2H), 3.46 (d, J=7.0 Hz, 2H), 3.19 (s, 1H), 3.07 (d, J=11.0 Hz, 1H), 2.20 (d, J=30.2 Hz, 1H), 1.95 (dd, J=23.1, 11.4 Hz, 2H), 1.81(d dd, J=29.8, 14.1, 7.0 Hz, 3H), 1.64-1.52 (m, 2H), 1.43 (dd, J=22.9, 14.7 Hz, 1H), 1.24 (s, 1H), 0.67(td, J=7.3, 3.5 Hz, 3H).

### Example 91 Preparation of ((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-7-fluoro-1-(prop-2-yn-1-yl)-1H-benzo[d]imidazol-5-yl)methanone

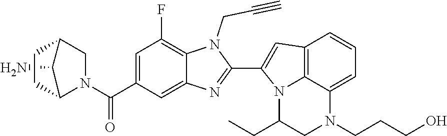 [SMILES: C#CCn1c(-c2cc3cccc4c3n2C(CC)CN4CCCO)nc2cc(C(=O)N3C[C@H]4CC[C@@H]3[C@@H]4N)cc(F)c21]

Prepared in analogous manner as for Example 69. LC-MS: (ESI⁺) m/z 554.8 [M+H]⁺. ¹H NMR (400 MHz, DMSO-d₆) δ 8.70 (s, 1H), 8.42 (s, 2H), 8.27 (d, J=31.4 Hz, 2H), 7.54-7.37 (m, 2H), 7.15 (t, J=7.9 Hz, 1H), 6.72 (d, J=7.7 Hz, 1H), 5.48 (s, 1H), 4.61 (s, 1H), 4.26 (s, 1H), 3.87 (s, 1H), 3.73-3.61 (m, 3H), 3.61-3.52 (m, 5H), 3.26 (s, 1H), 2.80 (s, 2H), 2.67 (d, J=31.2 Hz, 1H), 2.11-1.94 (m, 3H), 1.84(t dd, J=19.6, 13.2, 6.4 Hz, 4H), 1.67 (s, 1H), 1.23 (s, 1H), 0.98 (t, J=7.4 Hz, 3H).

### Example 92 Preparation of ((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-7-fluoro-1-(oxetan-3-ylmethyl)-1H-benzo[d]imidazol-5-yl)methanone

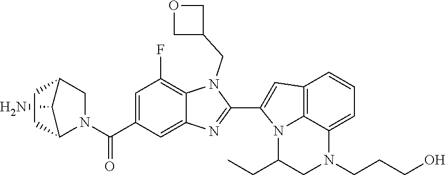 [SMILES: CCC1CN(CCCO)c2cccc3cc(-c4nc5cc(C(=O)N6C[C@H]7CC[C@@H]6[C@@H]7N)cc(F)c5n4CC4COC4)n1c23]

Prepared in analogous manner as for Example 69. LC-MS (ESI⁺): m/z 586.8 [M+H]⁺.

### Example 93 ((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-7-fluoro-1-(furan-2-ylmethyl)-1H-benzo[d]imidazol-5-yl)methanone

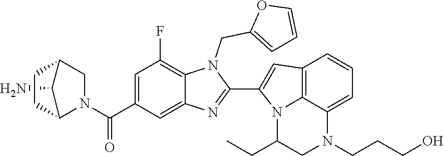 [SMILES: CCC1CN(CCCO)c2cccc3cc(-c4nc5cc(C(=O)N6C[C@H]7CC[C@@H]6[C@@H]7N)cc(F)c5n4Cc4ccco4)n1c23]

Prepared in analogous manner as for Example 69. LC-MS: (ESI⁺) m/z 597.1 [M+H]⁺.

### Example 94 Preparation of 3-(5-(5-((3R,5R)-3-amino-5-fluoropiperidine-1-carbonyl)-7-methoxy-1-methyl-1H-benzo[d]imidazol-2-yl)-3-ethyl-2,3-dihydro-1H-pyrrolo[1,2,3-de]quinoxalin-1-yl)propanamide

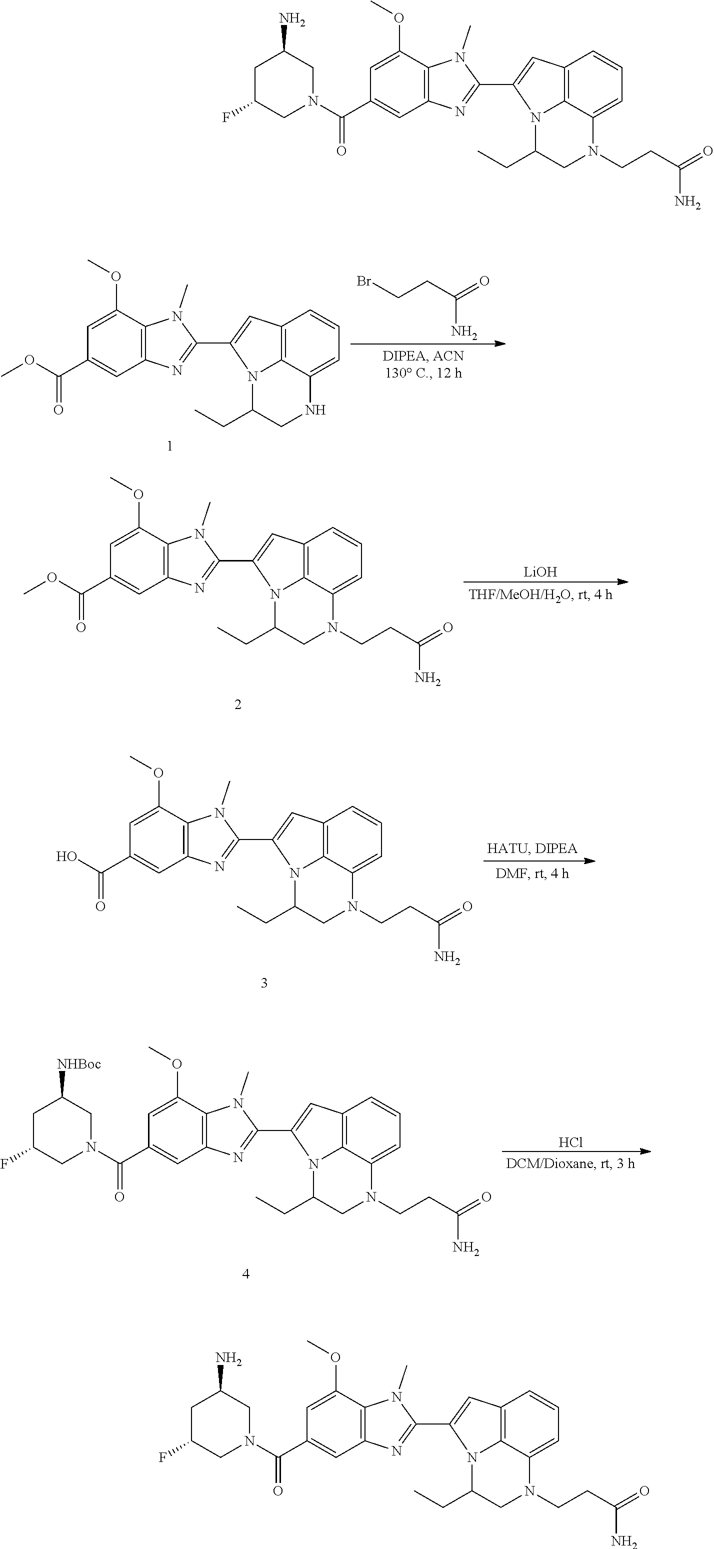

### Step 1:

To a solution of methyl 2-(11-ethyl-1,9-diazatricyclo[6.3.1.04,12]dodeca-2,4(12), 5,7-tetraen-2-yl)-7-methoxy-1-methyl-benzimidazole-5-carboxylate (200 mg, 494.49 μmol) in anhydrous ACN (3 mL) was added DIPEA (319.54 mg, 2.47 mmol, 430.64 μL) and 3-bromopropanamide (375.78 mg, 2.47 mmol) at RT. The reaction mixture was stirred at 130° C. for 12 h with by microwave, cooled to rt and concentrated in vacuo. The residue was purified by flash column chromatography on silica gel (elution with DCM/MeOH=1:0-20:1) to provide methyl 2-[9-(3-amino-3-oxo-propyl)-11-ethyl-1,9-diazatricyclo[6.3.1.04,12]dodeca-2,4(12), 5,7-tetraen-2-yl]-7-methoxy-1-methyl-benzimidazole-5-carboxylate (42 mg, 88.32 μmol, 17.86% yield) as a yellow solid, LC/MS (ESI⁺) [(M+H)⁺]: 475.6.

### Step 2:

To a stirred solution of methyl 2-[9-(3-amino-3-oxo-propyl)-11-ethyl-1,9-diazatricyclo[6.3.1.04,12]dodeca-2,4(12), 5,7-tetraen-2-yl]-7-methoxy-1-methyl-benzimidazole-5-carboxylate (42 mg, 88.32 μmol) in THF (1 mL), MeOH (0.5 mL) was added LiOH aqueous solution (1.0 M, 0.26 mL). The mixture was stirred at RT for 4 h, acidified to pH 5-6 with 3M hydrochloric acid aqueous solution, and extracted with EA(10*3 mL). The organic phase was dried anhydrous sodium sulfate and filtered. The filtrate was concentrated in vacuo to give 2-[9-(3-amino-3-oxo-propyl)-11-ethyl-1,9-diazatricyclo[6.3.1.04,12]dodeca-2,4(12), 5,7-tetraen-2-yl]-7-methoxy-1-methyl-benzimidazole-5-carboxylic acid (30 mg, 65.00 μmol, 73.60% yield) as a white solid. LC/MS (ESI⁺) [(M+H)⁺]: 461.5.

### Step 3:

To a solution of 2-[9-(3-amino-3-oxo-propyl)-11-ethyl-1,9-diazatricyclo[6.3.1.04,12]dodeca-2,4(12), 5,7-tetraen-2-yl]-7-methoxy-1-methyl-benzimidazole-5-carboxylic acid (30.0 mg, 65.00 μmol) in DMF (2 mL), DIPEA (25.20 mg, 195.01 μmol, 33.97 μL), HATU (49.43 mg, 130.01 μmol), tert-butyl N-[(3R,5R)-5-fluoro-3-piperidyl]carbamate (17.03 mg, 78.00 μmol) was added into the mixture, the mixture was stirred at RT 16 h. LC-MS showed the starting material was consumed and the desired mass was detected. After cooling to RT, and the mixture was diluted with EA and washed with brine and dried over Na₂SO₄. After filtration and evaporation of the solvent in vacuo, and the residue was purified by silica gel flash column chromatography(elution with DCM/MeOH=1:0-15:1) to give tert-butyl N-[(3R,5R)-1-[2-[9-(3-amino-3-oxo-propyl)-11-ethyl-1,9-diazatricyclo[6.3.1.04,12]dodeca-2,4(12), 5,7-tetraen-2-yl]-7-methoxy-1-methyl-benzimidazole-5-carbonyl]-5-fluoro-3-piperidyl]carbamate (25 mg, 37.78 μmol, 58.12% yield) as a yellow solid. LC/MS (ESI⁺) [(M+H)⁺]: 662.8.

### Step 4:

To a solution of tert-butyl N-[(3R,5R)-1-[2-[9-(3-amino-3-oxo-propyl)-11-ethyl-1,9-diazatricyclo[6.3.1.04,12]dodeca-2,4(12), 5,7-tetraen-2-yl]-7-methoxy-1-methyl-benzimidazole-5-carbonyl]-5-fluoro-3-piperidyl]carbamate (25 mg, 37.78 μmol) in DCM (1 mL). The mixture was stirred at 25° C. for 3 h. After completion of the reaction as judged by LC/MS. The mixture was concentrated under reduced pressure. The reaction mixture was adjusted pH=8 with saturated Na₂CO₃ solution, The mixture was extracted with DCM (30 mL*3). The organic layer was dried over anhydrous sodium sulfate. The combined organic layer was concentrated in vacuo, and the residue was purified by silica gel flash column chromatography(elution with DCM/MeOH=1:0-10:1) to give 3-[2-[5-[(3R,5R)-3-amino-5-fluoro-piperidine-1-carbonyl]-7-methoxy-1-methyl-benzimidazol-2-yl]-11-ethyl-1,9-diazatricyclo[6.3.1.04,12]dodeca-2,4(12), 5,7-tetraen-9-yl]propanamide (6.0 mg, 10.68 μmol) as a white solid, LC/MS (ESI⁺) [(M+H)⁺]: 561.8. ¹H NMR (400 MHz, DMSO-d₆) δ 8.20 (s, 2H), 7.36 (s, 1H), 7.01 (s, 1H), 6.98-6.93 (m, 2H), 6.88 (s, 1H), 6.44 (d, J=4.8 Hz, 1H), 5.22 (s, 1H), 5.01 (d, J=45.9 Hz, 1H), 4.22 (s, 3H), 4.00 (s, 3H), 3.72-3.50 (m, 6H), 2.47-2.33 (m, 6H), 1.90 (d, J=41.6 Hz, 3H), 1.58 (d, J=7.2 Hz, 3H), 1.33 (d, J=14.7 Hz, 1H), 0.67 (t, J=7.3 Hz, 3H).

### Example 95 Preparation of ((3R,5R)-3-amino-5-fluoropiperidin-1-yl)(2-(3-ethyl-1-(3-hydroxypropyl)-2,3-dihydro-1H-pyrrolo[1,2,3-de]quinoxalin-5-yl)-7-methoxy-1-(prop-2-yn-1-yl)-1H-benzo[d]imidazol-5-yl)methanone

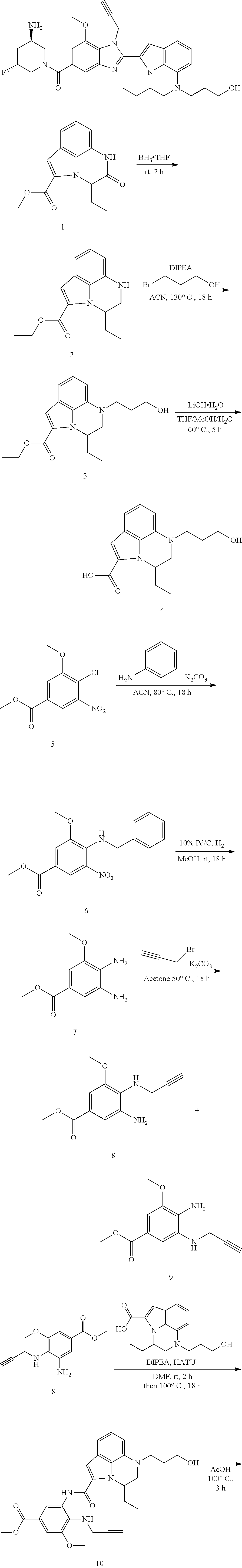

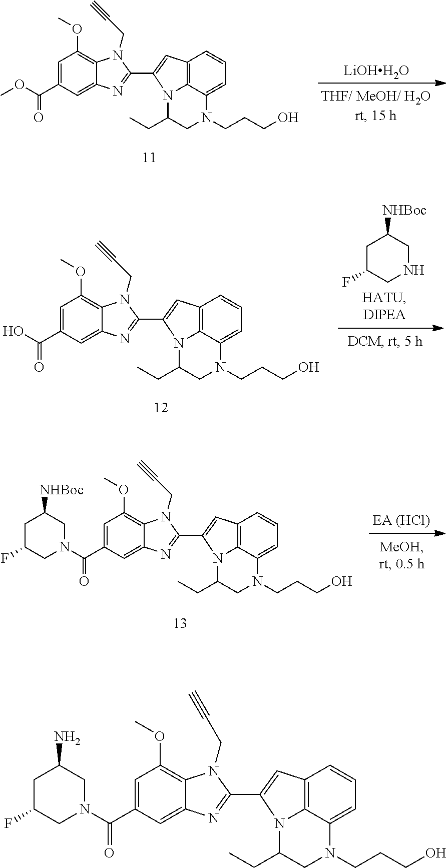 [SMILES: C#CCn1c(-c2cc3cccc4c3n2C(CC)CN4CCCO)nc2cc(C(=O)OC)cc(OC)c21 ; "LiOH·H2O" ; C#CCn1c(-c2cc3cccc4c3n2C(CC)CN4CCCO)nc2cc(C(=O)O)cc(OC)c21 ; CC(C)(C)OC(=O)N[C@H]1CNC[C@H](F)C1 ; C#CCn1c(-c2cc3cccc4c3n2C(CC)CN4CCCO)nc2cc(C(=O)N3C[C@H](F)C[C@@H](NC(=O)OC(C)(C)C)C3)cc(OC)c21 ; C#CCn1c(-c2cc3cccc4c3n2C(CC)CN4CCCO)nc2cc(C(=O)N3C[C@H](N)C[C@@H](F)C3)cc(OC)c21]

### Step 1:

A mixture of ethyl 11-ethyl-10-oxo-1,9-diazatricyclo[6.3.1.04,12]dodeca-2,4(12), 5,7-tetraene-2-carboxylate (2.4 g, 8.81 mmol), 1M borane tetrahydrofuran (17.62 mmol, 17.6 mL) was stirred for 2 h under an atmosphere of N₂. After the reaction was completed. The reaction mixture was quenched with H₂O (20 mL) and extracted with EtOAc (20 mL*2). Combined organic extracts were washed with brine (20 mL), dried over sodium sulfate and evaporated to give the crude product. The crude material was purified by flash column chromatography on silica gel (5-40% ethyl acetate/heptane) to afford ethyl 11-ethyl-1,9-diazatricyclo[6.3.1.04,12]dodeca-2,4(12), 5,7-tetraene-2-carboxylate (1.1 g, 4.26 mmol, 48.34% yield). LC/MS (ESI⁺) [(M+H)⁺]: 258.8.

### Step 2:

To a solution of ethyl 11-ethyl-1,9-diazatricyclo[6.3.1.04,12]dodeca-2,4(12), 5,7-tetraene-2-carboxylate (1.1 g, 4.26 mmol), 3-Bromo-1-propanol (2.96 g, 21.29 mmol, 1.86 mL) in acetonitrile, anhydrous, 99.8+% (10 mL) was added N,N-Diisopropylethylamine (2.75 g, 21.29 mmol, 3.71 mL). The resulting mixture was heated at 130° C. in a sealed tube for 18 h. The reaction was allowed to cool to rt and concentrated in vacuo. The crude material was purified by flash column chromatography on silica gel (5-60% ethyl acetate/heptane) to give ethyl 11-ethyl-9-(3-hydroxypropyl)-1,9-diazatricyclo[6.3.1.04,12]dodeca-2,4(12), 5,7-tetraene-2-carboxylate (1.0 g, 3.16 mmol, 74.22% yield). LC/MS (ESI⁺) [(M+H)⁺]: 316.8.

### Step 3:

To a solution of ethyl 11-ethyl-9-(3-hydroxypropyl)-1,9-diazatricyclo[6.3.1.04,12]dodeca-2,4(12), 5,7-tetraene-2-carboxylate (1.0 g, 3.16 mmol) in THF (6 mL), MeOH (2 mL) was added a solution of Lithium hydroxide monohydrate (397.86 mg, 9.48 mmol) in water (2 mL) and the resulting mixture was stirred at 60° C. for 5 h. The reaction crude was concentrated in vacuo and taken up in water (8 mL), acidified with 2N aqueous hydrochloric acid until no further precipitation was observed. The resulting suspension was allowed to stirr for 30 min and filtered through filter paper. The resulting solid was dried to afford 11-ethyl-9-(3-hydroxypropyl)-1,9-diazatricyclo[6.3.1.04,12]dodeca-2,4(12), 5,7-tetraene-2-carboxylic acid (900 mg, 3.12 mmol, 98.76% yield) as a greenish gray solid. LC/MS (ESI⁺) [(M+H)⁺]: 288.8.

### Step 4:

To a stirred solution of methyl 4-chloro-3-methoxy-5-nitro-benzoate (2.0 g, 8.14 mmol) in Acetonitrile, anhydrous, 99.8+% (12 mL) was added phenylmethanamine (1.75 g, 16.29 mmol) and potassium carbonate (1.69 g, 12.21 mmol). The mixture was stirred under nitrogen at 80° C. for 18 h, filtered and concentrated in vacuo. The residue was purified by flash column chromatography on silica gel (1-40% ethyl acetate/heptane) to afford methyl 4-(benzylamino)-3-methoxy-5-nitro-benzoate (2.5 g, 7.90 mmol, 97.06% yield) as an orange red solid. LC/MS (ESI⁺) [(M+H)⁺]: 316.8.

### Step 5:

To a flask containing methyl 4-(benzylamino)-3-methoxy-5-nitro-benzoate (2.5 g, 7.90 mmol) in MeOH (50 mL) were added 10% Pd/C (0.25 g, 50% Wt). The solution was degassed by H2 gas balloon. The mixture was stirred for 18 h. The reaction was completed and was filtered through a pad of Celite, which was washed with MeOH (3×10 mL). The combined solution was concentrated in vacuo to obtain methyl 3,4-diamino-5-methoxy-benzoate (1.5 g, 7.65 mmol, 96.73% yield) as a brown solid. LC/MS (ESI⁺) [(M+H)⁺]: 196.8.

### Step 6:

To a solution of methyl 3,4-diamino-5-methoxy-benzoate (0.67 g, 3.41 mmol) in Acetone (10 mL) was added potassium carbonate (471.96 mg, 3.41 mmol) followed by 3-Bromopropyne (507.79 mg, 3.41 mmol, 80% purity). The mixture was stirred at 50° C. for 24 h, cooled down to RT, filtered and concentrated in vacuo. The crude product was purified by flash column chromatography on silica gel (0-40% ethyl acetate/heptane) to give methyl 3-amino-5-methoxy-4-(prop-2-ynylamino)benzoate (200 mg, 853.79 μmol, 25.00% yield) as a brown solid. LC/MS (ESI⁺) [(M+H)⁺]: 234.8.

### Step 7:

To a solution of methyl 3-amino-5-methoxy-4-(prop-2-ynylamino)benzoate (200 mg, 853.79 μmol) and 11-ethyl-9-(3-hydroxypropyl)-1,9-diazatricyclo[6.3.1.04,12]dodeca-2,4(12), 5,7-tetraene-2-carboxylic acid (246.18 mg, 853.79 μmol) in DMF (5 mL) at RT was added HATU (422.03 mg, 1.11 mmol) and N,N-Diisopropylethylamine (331.03 mg, 2.56 mmol, 446.13 μL). The reaction mixture was stirred at RT for 2 h and then heated to 100° C. for 15 h. After the reaction was completed, quenched with H₂O (15 mL) and extracted with CH₂Cl₂ (30 mL*2). Combined organic extracts were washed with brine (20 mL), dried over sodium sulfate, and evaporated to give the crude product. The crude product was purified by flash column chromatography on silica gel using 2-20% MeOH in CH₂Cl₂ to afford title product methyl 3-[[11-ethyl-9-(3-hydroxypropyl)-1,9-diazatricyclo[6.3.1.04,12]dodeca-2,4(12), 5,7-tetraene-2-carbonyl]amino]-5-methoxy-4-(prop-2-ynylamino)benzoate (65 mg, 128.82 μmol, 15.09% yield) as a bluish white solid. LC/MS (ESI⁺) [(M+H)⁺]: 504.8.

### Step 8:

A mixture of methyl 3-[[11-ethyl-9-(3-hydroxypropyl)-1,9-diazatricyclo[6.3.1.04,12]dodeca-2,4(12), 5,7-tetraene-2-carbonyl]amino]-5-methoxy-4-(prop-2-ynylamino)benzoate (65 mg, 128.82 μmol) and acetic acid (3 mL) was stirred at 100° C. for 3 h under an atmosphere of N₂. until the reaction was completed. The reaction mixture was concentrated in vacuo and diluted with EtOAc (10 mL), NaHCO₃(aq) (6 mL), extracted with EtOAc (10 mL*2). The combined organic extracts were washed with brine (20 mL), dried over sodium sulfate, and evaporated to give the crude product. The crude product was purified by flash column chromatography on silica gel using 1-70% EtOAc in hexane to afford title product methyl 2-[11-ethyl-9-(3-hydroxypropyl)-1,9-diazatricyclo[6.3.1.04,12]dodeca-2,4(12), 5,7-tetraen-2-yl]-7-methoxy-1-prop-2-ynyl-benzimidazole-5-carboxylate (10 mg, 20.55 μmol, 15.95% yield). LC-MS (ESI): m/z 486.8 [M+H]⁺.

### Step 9:

To a solution of methyl 2-[11-ethyl-9-(3-hydroxypropyl)-1,9-diazatricyclo[6.3.1.04,12]dodeca-2,4(12), 5,7-tetraen-2-yl]-7-methoxy-1-prop-2-ynyl-benzimidazole-5-carboxylate (10 mg, 20.55 μmol) in THF (2.0 mL) was added a solution of Lithium hydroxide monohydrate, (2.59 mg, 61.66 μmol) in water (0.5 mL) and the resulting mixture was stirred at RT for 15 h. The reaction crude was concentrated in vacuo and taken up in water (5 mL), acidified with 2N aqueous hydrochloric acid and extracted with MeOH/CH₂Cl₂(20 mL*2). The combined organic layer was dried over anhydrous sodium sulphate and filtered. The filtrate evaporated under vacuum to afford the product 2-[11-ethyl-9-(3-hydroxypropyl)-1,9-diazatricyclo[6.3.1.04,12]dodeca-2,4(12), 5,7-tetraen-2-yl]-7-methoxy-1-prop-2-ynyl-benzimidazole-5-carboxylic acid (5 mg, 10.58 μmol, 51.48% yield) as an off-white solid. LC/MS (ESI⁺) [(M+H)⁺]: 472.8.

### Step 10:

To a solution of 2-[11-ethyl-9-(3-hydroxypropyl)-1,9-diazatricyclo[6.3.1.04,12]dodeca-2,4(12), 5,7-tetraen-2-yl]-7-methoxy-1-prop-2-ynyl-benzimidazole-5-carboxylic acid (5 mg, 10.58 μmol) and tert-butyl N-[(3R,5R)-5-fluoro-3-piperidyl]carbamate (2.77 mg, 12.70 μmol) in CH₂Cl₂ (3 mL)) at RT was added HATU (5.23 mg, 13.76 μmol) and N,N-Diisopropylethylamine (6.84 mg, 52.91 μmol, 9.22 μL). The reaction mixture was stirred at RT for 2 h. After the completion of the reaction, the mixture was quenched with H₂O (8 mL) and extracted with CH₂Cl₂ (2×20 mL). The combined organic extracts were washed with brine (20 mL), dried over sodium sulfate and evaporated to give the crude product. The crude product was purified by flash column chromatography on silica gel using 2-20% MeOH in CH₂Cl₂ to afford title product tert-butyl N-[(3R,5R)-1-[2-[11-ethyl-9-(3-hydroxypropyl)-1,9-diazatricyclo[6.3.1.04,12]dodeca-2,4(12), 5,7-tetraen-2-yl]-7-methoxy-1-prop-2-ynyl-benzimidazole-5-carbonyl]-5-fluoro-3-piperidyl]carbamate (5.0 mg, 7.43 μmol, 70.24% yield) as a bluish white solid. LC/MS (ESI⁺) [(M+H)⁺]: 672.8.

### Step 11:

To a stirred solution tert-butyl N-[(3R,5R)-1-[2-[11-ethyl-9-(3-hydroxypropyl)-1,9-diazatricyclo[6.3.1.04,12]dodeca-2,4(12), 5,7-tetraen-2-yl]-7-methoxy-1-prop-2-ynyl-benzimidazole-5-carbonyl]-5-fluoro-3-piperidyl]carbamate (5.0 mg, 7.43 μmol) in MeOH (0.5 mL), was added 4M HCl in EtOAc (2 mL). The reaction mixture was stirred at RT for 0.5 h. The reaction mixture was evaporated to afford the crude product and then pre-HPLC to give [(3R,5R)-3-amino-5-fluoro-1-piperidyl]-[2-[11-ethyl-9-(3-hydroxypropyl)-1,9-diazatricyclo[6.3.1.04,12]dodeca-2,4(12), 5,7-tetraen-2-yl]-7-methoxy-1-prop-2-ynyl-benzimidazol-5-yl]methanone (2.5 mg, 4.37 μmol, 58.74% yield) as a white solid. LC/MS (ESI⁺) [(M+H)⁺]: 572.8.

### Example 96 ((1R,4R,7R)-7-amino-2-azabicyclo[2.2.1]heptan-2-yl)(1-ethyl-2-(3-ethyl-1-(3-hydroxypropyl)-2,3-dihydro-1H-pyrrolo[1,2,3-de]quinoxalin-5-yl)-7-fluoro-1H-benzo[d]imidazol-5-yl)methanone

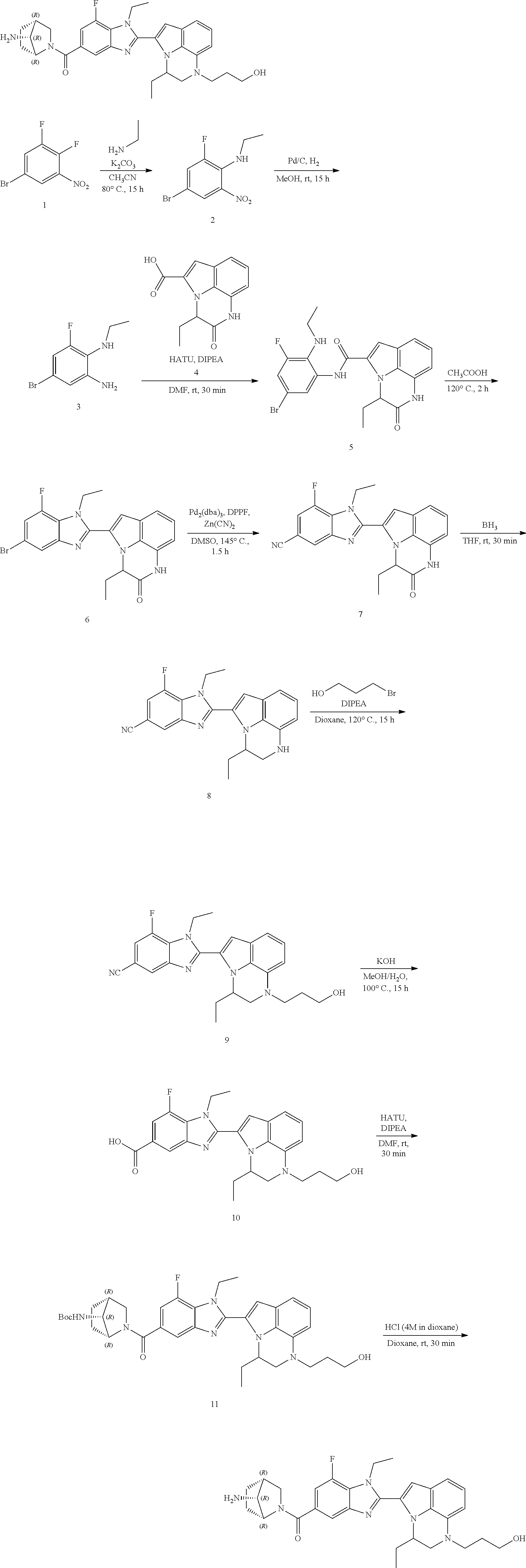

### Step 1:

A mixture of ethanamine (1.4 g, 31.51 mmol, 1.77 mL), 5-bromo-1,2-difluoro-3-nitro-benzene (5.0 g, 21.01 mmol) and potassium carbonate (2.9 g, 21.01 mmol, 1.27 mL) was dissolved in acetonitrile (28.8 mL). It was stirred at 80° C. for 15 h. The reaction was diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give 4-bromo-N-ethyl-2-fluoro-6-nitro-aniline (4.0 g, 15.21 mmol, 72.37% yield) as a yellow solid. LC/MS (ESI⁺) [(M+H)⁺]: 262.8.

### Step 2:

A mixture of 4-bromo-N-ethyl-2-fluoro-6-nitro-aniline (4.0 g, 15.21 mmol) and H₂ (ballon) in methanol (30 mL) was hydrogenated with palladium 10% on carbon (413.6 mg, 3.90 mmol) at ambient temperature for overnihgt. The mixture was filtered over Celite to remove Pd/C, and the solvent was evaporated to give the desired product 5-bromo-N2-ethyl-3-fluoro-benzene-1,2-diamine (3.0 g, 12.87 mmol, 84.65% yield) as a colorless solid. LC/MS (ESI⁺) [(M+H)⁺]: 232.8.

### Step 3:

A mixture of 5-bromo-N2-ethyl-3-fluoro-benzene-1,2-diamine (2.9 g, 12.61 mmol), 11-ethyl-10-oxo-1,9-diazatricyclo[6.3.1.04,12]dodeca-2,4(12), 5,7-tetraene-2-carboxylic acid (2.8 g, 11.46 mmol), HATU (6.5 g, 17.20 mmol) and DIPEA (2.9 g, 22.93 mmol, 3.99 mL) was dissolved in DMF (7.43 mL). The resulting mixture was stirred at 80° C. for 4 h. The reaction was diluted with EtOAc (50 mL) and washed with water (25 mL). The organic layer was dried over sodium sulfate, filtered and concentrated in vacuo. The crude material was purified by flash column chromatogrpahy on silica gel (eluting with EA/PE from 0 to 100%) to give N-[5-bromo-2-(ethylamino)-3-fluoro-phenyl]-11-ethyl-10-oxo-1,9-diazatricyclo[6.3.1.04,12]dodeca-2,4(12), 5,7-tetraene-2-carboxamide (3.0 g, 6.53 mmol, 56.97% yield) as a yellow solid. LC/MS (ESI⁺) [(M+H)⁺]: 440.8.

### Step 4:

N-[5-bromo-2-(ethylamino)-3-fluoro-phenyl]-11-ethyl-10-oxo-1,9-diazatricyclo[6.3.1.04,12]dodeca-2,4(12), 5,7-tetraene-2-carboxamide (3.0 g, 6.53 mmol) was dissolved in acetic acid (15 mL) and stirred at 120° C. for 2 h. After cooling the reaction to RT, and removal the solvent in vacuo, the residue was purified by flash column chromatography on silica gel (eluting with EA/PE from 0 to 50%) to give 2-(5-bromo-1-ethyl-7-fluoro-benzimidazol-2-yl)-11-ethyl-1,9-diazatricyclo[6.3.1.04,12]dodeca-2,4(12), 5,7-tetraen-10-one (2.6 g, 5.89 mmol, 90.20% yield) as a white solid. LC/MS (ESI⁺) [(M+H)⁺]: 440.8.

### Step 5:

A mixture of dicyanozinc (276.1 mg, 2.35 mmol, 149.09 μL), N-[5-bromo-2-(ethylamino)-3-fluoro-phenyl]-11-ethyl-10-oxo-1,9-diazatricyclo[6.3.1.04,12]dodeca-2,4(12), 5,7-tetraene-2-carboxamide (3.0 g, 6.53 mmol), 1,1′-Bis(diphenylphosphino)ferrocene (724.2 mg, 1.31 mmol) and Tris(Dibenzylideneacetone)dipalladium (0) (598.1 mg, 653.15 μmol) was dissolved in DMSO (30 mL). The resulting mixture was stirred at 140° C. under the atmosphere of nitrogen for 2 h in microwave reactor. The mixture was purified by flash coulmn chromatography on silica gel (eluting with PE/EA from 0 to 50%) to give 2-(5-bromo-1-ethyl-7-fluoro-benzimidazol-2-yl)-11-ethyl-1,9-diazatricyclo[6.3.1.04,12]dodeca-2,4(12), 5,7-tetraen-10-one (2.0 g, 4.53 mmol, 69.39% yield) as yellow liquid oil. LC/MS (ESI⁺) [(M+H)⁺]: 387.8.

### Step 6:

To a solution of 1-ethyl-2-(11-ethyl-10-oxo-1,9-diazatricyclo[6.3.1.04,12]dodeca-2,4(12), 5,7-tetraen-2-yl)-7-fluoro-benzimidazole-5-carbonitrile (1.7 g, 4.53 mmol) in anhydrous THF (10 mL) was added borane-tetrahydrofuran complex at 0° C. slowly. The reaction mixture was stirred at RT for 30 min, quenched with MeOH at 0° C. and concentrated in vacuo. The residue was diluted with 1 N HCl aqueous solution (1 mL), stirred at rt for 1 h, and basified with 1 N NaOH aqueous solution to pH ˜8. The resulting mixture was extrated with DCM (10 mL*3), and the combined organic phase was dried over anhydrous sodium sulfate and filtered. The filtrate was concentrated in vacuo and the residue was purified by silica gel flash column chromatography (eluting with DCM/MeOH=1:0-20:1) to give 1-ethyl-2-(11-ethyl-1,9-diazatricyclo[6.3.1.04,12]dodeca-2,4(12), 5,7-tetraen-2-yl)-7-fluoro-benzimidazole-5-carbonitrile (1.4 g, 3.75 mmol, 82.72% yield) as a yellow solid. LC/MS (ESI⁺) [(M+H)⁺]: 373.8.

### Step 7:

A mixture of 3-bromopropan-1-ol (2.6 g, 18.75 mmol, 1.64 mL), 1-ethyl-2-(11-ethyl-1,9-diazatricyclo[6.3.1.04,12]dodeca-2,4(12), 5,7-tetraen-2-yl)-7-fluoro-benzimidazole-5-carbonitrile (1.4 g, 3.75 mmol) and DIPEA (2.9 g, 22.49 mmol, 3.92 mL) was dissolved in acetonitrile (15 mL). The resulting mixture was stirred at 120° C. for 15 h in microwave reactor. After removal of the solvent in vacuo, the residue was purified by flash column chromatography on silica gel (eluting with EA/PE from 0 to 100) to give 1-ethyl-2-[11-ethyl-9-(3-hydroxypropyl)-1,9-diazatricyclo[6.3.1.04,12]dodeca-2,4(12), 5,7-tetraen-2-yl]-7-fluoro-benzimidazole-5-carbonitrile (1.2 g, 2.78 mmol, 74.18% yield) as liquid oil. LC/MS (ESI⁺) [(M+H)⁺]: 431.8.

### Step 8:

A mixture of 1-ethyl-2-[11-ethyl-9-(3-hydroxypropyl)-1,9-diazatricyclo[6.3.1.04,12]dodeca-2,4(12), 5,7-tetraen-2-yl]-7-fluoro-benzimidazole-5-carbonitrile (1.2 g, 2.78 mmol) and potassium hydroxide (467.2 mg, 8.34 mmol) was dissolved in methanol/water=1/1 (10 mL). The resulting mixture was stirred at 100° C. for overnight. Desired signal was found by LC/MS. The mixture was acidified with 3 mol/L hydrochloric acid and purified by pre-HPLC to give 1-ethyl-2-[11-ethyl-9-(3-hydroxypropyl)-1,9-diazatricyclo[6.3.1.04,12]dodeca-2,4(12), 5,7-tetraen-2-yl]-7-fluoro-benzimidazole-5-carboxylic acid (800.0 mg, 1.78 mmol, 63. % yield) as a yellow solid. LC/MS (ESI⁺) [(M+H)⁺]: 450.8.

### Step 9:

A mixture of 1-ethyl-2-[11-ethyl-9-(3-hydroxypropyl)-1,9-diazatricyclo[6.3.1.04,12]dodeca-2,4(12), 5,7-tetraen-2-yl]-7-fluoro-benzimidazole-5-carboxylic acid (50 mg, 110.99 μmol), tert-butyl N-[(1R,4R,7R)-2-azabicyclo[2.2.1]heptan-7-yl]carbamate (PharmaBlock) (25.9 mg, 122.09 μmol), DIPEA (28.7 mg, 221.97 μmol, 38.66 μL) and HATU (63.3 mg, 166.48 μmol) was dissolved in DMF (2 mL). The resulting mixture was stirred at 50° C. for 10 min. The reaction mixture was diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give tert-butyl N-[(1R,4R,7R)-2-[1-ethyl-2-[11-ethyl-9-(3-hydroxypropyl)-1,9-diazatricyclo[6.3.1.04,12]dodeca-2,4(12), 5,7-tetraen-2-yl]-7-fluoro-benzimidazole-5-carbonyl]-2-azabicyclo[2.2.1]heptan-7-yl]carbamate (30.0 mg, 46.53 μmol, 41.92% yield) as yellow oil. LC/MS (ESI⁺) [(M+H)⁺]: 644.8.

### Step 10:

tert-butyl N-[(1R,4R,7R)-2-[1-ethyl-2-[11-ethyl-9-(3-hydroxypropyl)-1,9-diazatricyclo[6.3.1.04,12]dodeca-2,4(12), 5,7-tetraen-2-yl]-7-fluoro-benzimidazole-5-carbonyl]-2-azabicyclo[2.2.1]heptan-7-yl]carbamate (30.0 mg, 46.53 μmol) was dissolved in HCl (4M)/Dioane=1/2 (3 mL). The resulgint mixture was stirred at rt for 30 min. After removal of the solvent in vacuo and purified by pre-HPLC to afford [(1R,4R,7R)-7-amino-2-azabicyclo[2.2.1]heptan-2-yl]-[1-ethyl-2-[11-ethyl-9-(3-hydroxypropyl)-1,9-diazatricyclo[6.3.1.04,12]dodeca-2,4(12), 5,7-tetraen-2-yl]-7-fluoro-benzimidazol-5-yl]methanone (14.0 mg, 25.70 μmol, 55.24% yield) as a white solid. LC/MS (ESI⁺) [(M+H)⁺]: 544.8. ¹H NMR (400 MHz, DMSO-d₆) δ 7.62 (dd, J=4.5, 1.2 Hz, 1H), 7.23 (d, J=11.9 Hz, 1H), 7.01-6.91 (m, 3H), 6.44 (dd, J=6.8, 1.5 Hz, 1H), 5.22 (s, 1H), 4.57 (dq, J=14.6, 7.3 Hz, 2H), 3.75 (d, J=11.7 Hz, 1H), 3.60-3.53 (m, 4H), 3.52-3.40 (m, 3H), 3.21 (s, 1H), 3.09 (d, J=11.0 Hz, 1H), 2.22 (s, 1H), 2.00-1.72 (m, 5H), 1.72-1.48 (m, 6H), 1.44 (t, J=9.7 Hz, 1H), 0.70 (td, J=7.5, 3.5 Hz, 3H).

### Example 97 ((1R,4R,7R)-7-amino-2-azabicyclo[2.2.1]heptan-2-yl)(1-ethyl-2-((R)-3-ethyl-1-(3-hydroxypropyl)-2,3-dihydro-1H-pyrrolo[1,2,3-de]quinoxalin-5-yl)-7-fluoro-1H-benzo[d]imidazol-5-yl)methanone and Example 98 ((1R,4R,7R)-7-amino-2-azabicyclo[2.2.1]heptan-2-yl)(1-ethyl-2-((S)-3-ethyl-1-(3-hydroxypropyl)-2,3-dihydro-1H-pyrrolo[1,2,3-de]quinoxalin-5-yl)-7-fluoro-1H-benzo[d]imidazol-5-yl)methanone

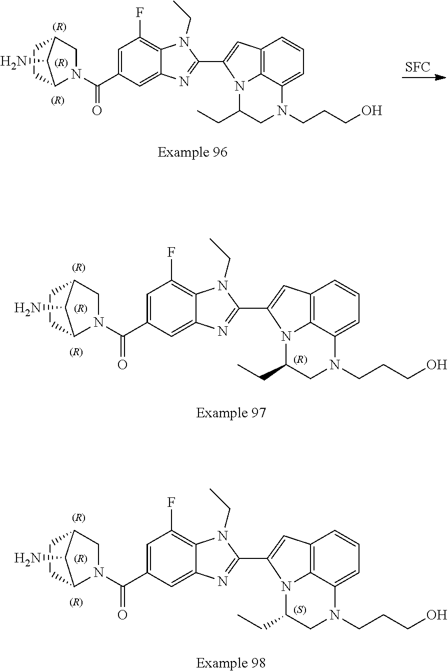

((1R,4R,7R)-7-amino-2-azabicyclo[2.2.1]heptan-2-yl)(1-ethyl-2-(3-ethyl-1-(3-hydroxypropyl)-2,3-dihydro-1H-pyrrolo[1,2,3-de]quinoxalin-5-yl)-7-fluoro-1H-benzo[d]imidazol-5-yl)methanone (200 mg, 0.37 mmol) was chiral separated by SFC with mobile phase (CO₂/MEOH (0.2% Methanol Ammonia)=45/55) (Column: OZ 4.6*100 mm 5 um) (Flow Rate: 120 g/min) to give synthetic Example 97 (60 mg, 11.03%) as an off-white solid. ¹H NMR (400 MHz, DMSO-d₆) δ 7.61 (s, 1H), 7.24 (d, J=11.8 Hz, 1H), 7.01-6.90 (m, 3H), 6.44 (dd, J=6.8, 1.5 Hz, 1H), 5.22 (t, J=6.7 Hz, 1H), 4.60-4.53 (m, 2H), 3.77 (s, 1H), 3.55 (h, J=2.3 Hz, 4H), 3.50-3.44 (m, 3H), 3.23 (s, 1H), 3.10-3.04 (m, 1H), 2.26 (s, 1H), 1.99-1.74 (m, 5H), 1.62-1.43 (m, 6H), 1.24 (d, J=3.8 Hz, 1H), 0.68 (t, J=7.4 Hz, 3H) and Example 98 (50 mg, 9.19%) as an off-white solid. ¹H NMR (400 MHz, DMSO-d6) δ 7.62 (s, 1H), 7.23 (d, J=11.9 Hz, 1H), 7.00-6.91 (m, 3H), 6.44 (dd, J=6.8, 1.6 Hz, 1H), 5.21 (q, J=5.0 Hz, 1H), 4.56 (dq, J=14.6, 7.5 Hz, 2H), 3.77 (s, 1H), 3.55 (td, J=6.0, 2.1 Hz, 4H), 3.46 (t, J=7.2 Hz, 3H), 3.21 (s, 1H), 3.08 (d, J=10.7 Hz, 1H), 2.23 (s, 1H), 1.99-1.70 (m, 5H), 1.61-1.39 (m, 6H), 1.24 (d, J=3.5 Hz, 1H), 0.69 (t, J=7.5 Hz, 3H).

Based on the co-cystallization results of Example 71, it is believed that Example 98, which is more potent than Example 97 in the PAD4 biochemical assay, has the S configuration.

### Example 99 ((3R,5R)-3-amino-5-fluoropiperidin-1-yl)(1-ethyl-2-(3-ethyl-1-(3-hydroxypropyl)-2,3-dihydro-1H-pyrrolo[1,2,3-de]quinoxalin-5-yl)-7-fluoro-1H-benzo[d]imidazol-5-yl)methanone

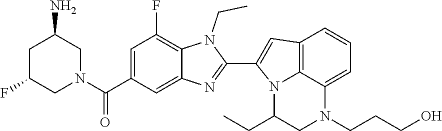 [SMILES: CCC1CN(CCCO)c2cccc3cc(-c4nc5cc(C(=O)N6C[C@H](N)C[C@@H](F)C6)cc(F)c5n4CC)n1c23]

Prepared in analogous manner as for Example 96. LC/MS(ESI⁺) [(M+H)⁺]: 550.8.

### Example 100 ((1R,4R,7R)-7-amino-2-azabicyclo[2.2.1]heptan-2-yl)(1-ethyl-2-(3-ethyl-1-(3-methoxypropyl)-2,3-dihydro-1H-pyrrolo[1,2,3-de]quinoxalin-5-yl)-7-fluoro-1H-benzo[d]imidazol-5-yl)methanone

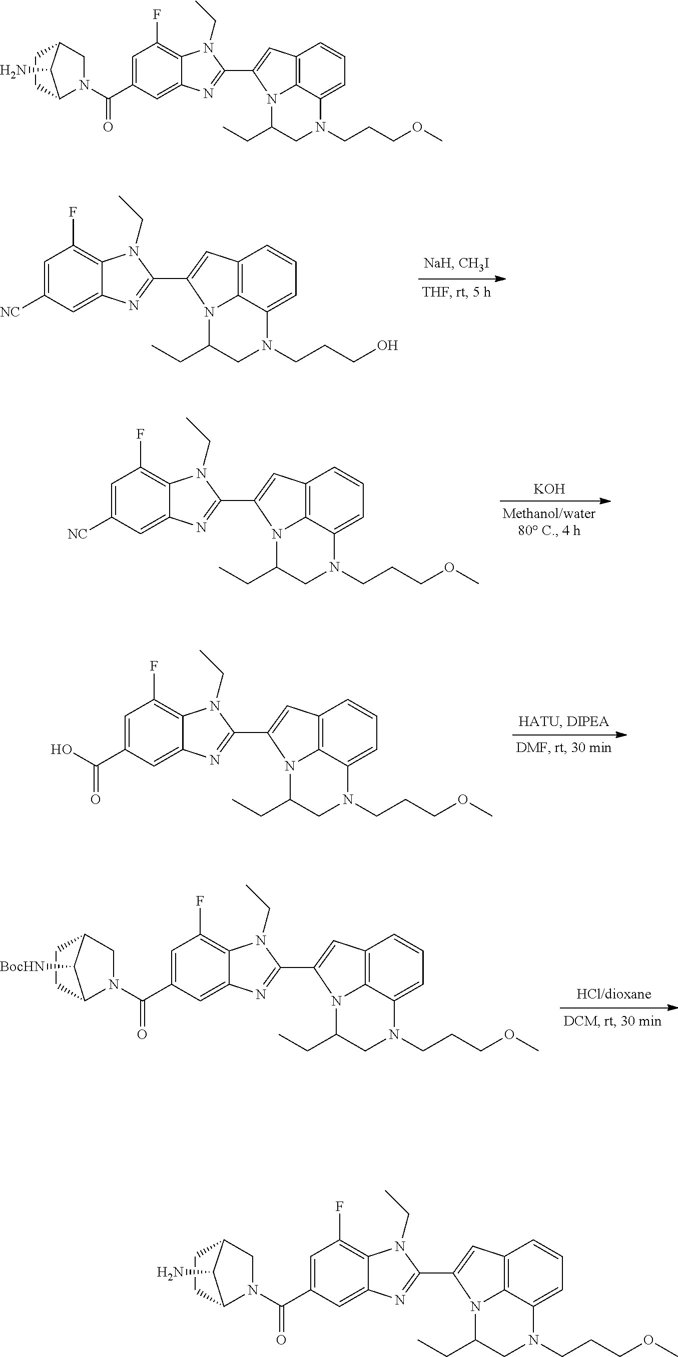

### Step 1: 1-ethyl-2-(3-ethyl-1-(3-methoxypropyl)-2,3-dihydro-1H-pyrrolo[1,2,3-de]quinoxalin-5-yl)-7-fluoro-1H-benzo[d]imidazole-5-carbonitrile

To a solution of 1-ethyl-2-[11-ethyl-9-(3-hydroxypropyl)-1,9-diazatricyclo[6.3.1.04,12]dodeca-2,4(12), 5,7-tetraen-2-yl]-7-fluoro-benzimidazole-5-carbonitrile (50.0 mg, 115.87 μmol) in THF (2 mL) was added iodomethane (24.7 mg, 173.81 μmol, 10 μL). The resulting mixture was stirred at 25° C. for 5 h. Desired signal was found by LC/MS. The mixture was acidified with 3 mol/L hydrochloric acid. The mixture was purified by pre-HPLC to give 1-ethyl-2-[11-ethyl-9-(3-methoxypropyl)-1,9-diazatricyclo[6.3.1.04,12]dodeca-2,4(12), 5,7-tetraen-2-yl]-7-fluoro-benzimidazole-5-carbonitrile (40.0 mg, 89.78 μmol, 77.48% yield) as a yellow solid. LC/MS(ESI⁺) [(M+H)]: 445.8

### Step 2: 1-ethyl-2-(3-ethyl-1-(3-methoxypropyl)-2,3-dihydro-1H-pyrrolo[1,2,3-de]quinoxalin-5-yl)-7-fluoro-1H-benzo[d]imidazole-5-carboxylic acid

A mixture of 1-ethyl-2-[11-ethyl-9-(3-methoxypropyl)-1,9-diazatricyclo[6.3.1.04,12]dodeca-2,4(12), 5,7-tetraen-2-yl]-7-fluoro-benzimidazole-5-carbonitrile (40.0 mg, 89.78 μmol) and Potassium hydroxide (10.0 mg, 179.56 μmol) was dissolved in methanol/water=1/1 (2 mL) and stirred at 100° C. for overnight. Desired signal was found by LC/MS. The mixture was acidified with 3 mol/L hydrochloric acid. The mixture was purified by pre-HPLC to give 1-ethyl-2-[11-ethyl-9-(3-methoxypropyl)-1,9-diazatricyclo[6.3.1.04,12]dodeca-2,4(12), 5,7-tetraen-2-yl]-7-fluoro-benzimidazole-5-carboxylic acid (30.0 mg, 64.58 μmol, 71.93% yield) as a yellow solid. LC/MS(ESI⁺) [(M+H)⁺]: 464.8

### Step 3: tert-butyl ((1R,4R,7R)-2-(1-ethyl-2-(3-ethyl-1-(3-methoxypropyl)-2,3-dihydro-1H-pyrrolo[1,2,3-de]quinoxalin-5-yl)-7-fluoro-1H-benzo[d]imidazole-5-carbonyl)-2-azabicyclo[2.2.1]heptan-7-yl)carbamate

A mixture of 1-ethyl-2-[11-ethyl-9-(3-methoxypropyl)-1,9-diazatricyclo[6.3.1.04,12]dodeca-2,4(12), 5,7-tetraen-2-yl]-7-fluoro-benzimidazole-5-carboxylic acid (30.0 mg, 64.58 μmol), tert-butyl N-[(1R,4R,7R)-2-azabicyclo[2.2.1]heptan-7-yl]carbamate (16.4 mg, 77.50 μmol), DIPEA (16.7 mg, 129.16 μmol, 22.50 μL) and HATU (36.8 mg, 96.87 μmol) was dissolved in DMF (2 mL). The resulting mixture was stirred at 25° C. for 10 min. Desired signal was found by LC/MS, SM consumed. The reaction was diluted with EtOAc (50 ml) and washed with water (25 ml). The organic layer was dried over sodium sulfate, filtered and concentrated in vacuo. The crude material was purified by flash column chromatogrpahy on silica gel (eluting with EA/PE from 0 to 100%) to give tert-butyl N-[(1R,4R,7R)-2-[1-ethyl-2-[11-ethyl-9-(3-methoxypropyl)-1,9-diazatricyclo[6.3.1.04,12]dodeca-2,4(12), 5,7-tetraen-2-yl]-7-fluoro-benzimidazole-5-carbonyl]-2-azabicyclo[2.2.1]heptan-7-yl]carbamate (20.0 mg, 30.36 μmol, 47.01% yield) as yellow oil. LC/MS(ESI⁺) [(M+H)⁺]: 658.8

### Step 4: ((1R,4R,7R)-7-amino-2-azabicyclo[2.2.1]heptan-2-yl)(1-ethyl-2-(3-ethyl-1-(3-methoxypropyl)-2,3-dihydro-1H-pyrrolo[1,2,3-de]quinoxalin-5-yl)-7-fluoro-1H-benzo[d]imidazol-5-yl)methanone

tert-butyl N-[(1R,4R,7R)-2-[1-ethyl-2-[11-ethyl-9-(3-methoxypropyl)-1,9-diazatricyclo[6.3.1.04,12]dodeca-2,4(12), 5,7-tetraen-2-yl]-7-fluoro-benzimidazole-5-carbonyl]-2-azabicyclo[2.2.1]heptan-7-yl]carbamate (20.0 mg, 30.36 μmol) was dissolved in HCl (4M)/Dioane=1/2 (3 mL). The resulting mixture was stirred at rt for 30 min. After removal of the solvent in vacuo, the residue was purified by pre-HPLC to afford [(1R,4R,7R)-7-amino-2-azabicyclo[2.2.1]heptan-2-yl]-[1-ethyl-2-[11-ethyl-9-(3-methoxypropyl)-1,9-diazatricyclo[6.3.1.04,12]dodeca-2,4(12), 5,7-tetraen-2-yl]-7-fluoro-benzimidazol-5-yl]methanone (8.0 mg, 14.32 μmol, 47.17% yield) as an off-white solid. LC/MS(ESI⁺) [(M+H)⁺]: 558.8. ¹H NMR (400 MHz, DMSO-d₆) δ 7.62 (d, J=4.4 Hz, 1H), 7.30-7.19 (m, 1H), 7.02-6.91 (m, 3H), 6.45-6.39 (m, 1H), 5.22 (s, 1H), 4.58 (s, 2H), 3.75 (d, J=11.7 Hz, 1H), 3.55 (d, J=7.6 Hz, 2H), 3.46 (td, J=6.4, 3.1 Hz, 5H), 3.28 (s, 3H), 3.20 (s, 1H), 3.11-3.02 (m, 2H), 2.21 (d, J=8.0 Hz, 1H), 1.99-1.84 (m, 4H), 1.72 (d, J=8.1 Hz, 1H), 1.62-1.49 (m, 5H), 0.69 (td, J=7.5, 3.4 Hz, 3H).

### Example 101 Preparation of ((1R,4R,7R)-7-amino-2-azabicyclo[2.2.1]heptan-2-yl)(2-(3-ethyl-1-(3-hydroxypropyl)-2,3-dihydro-1H-pyrrolo[1,2,3-de]quinoxalin-5-yl)-4-fluoro-1-(prop-2-yn-1-yl)-1H-benzo[d]imidazol-6-yl)methanone

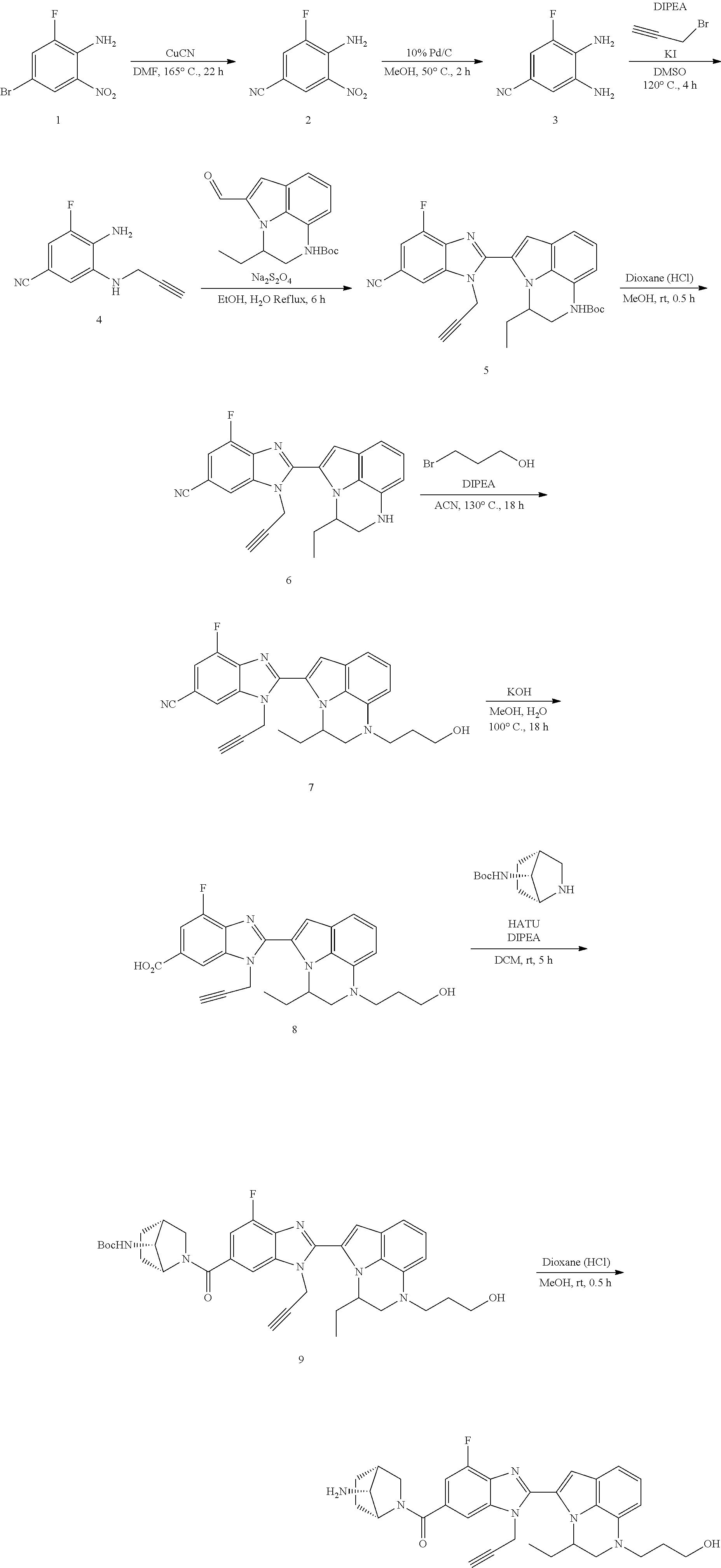

### Step 1:

To a solution of 4-bromo-2-fluoro-6-nitro-aniline (2.5 g, 10.64 mmol) in DMF (20 mL) was added Copper(I) Cyanide (1.91 g, 21.28 mmol, 652.55 μL). After stirring at 165° C. for 22 h, the reaction mixture was cooled down to RT, poured on to water (75 mL) and extracted with ethyl acetate (3×50 mL). The combined organic layers were washed with water (80 mL) and saturated aqueous sodium chloride (80 mL), dried over sodium sulfate and concentrated in vacuo. The crude product was purified by flash column chromatography on silica gel (0-20% ethyl acetate/heptane) to obtain 4-amino-3-fluoro-5-nitro-benzonitrile (1.2 g, 6.63 mmol, 62.28% yield) as a yellow solid. LC/MS(ESI⁺) [(M+H)⁺]: 181.8.

### Step 2:

A mixture of 4-amino-3-fluoro-5-nitro-benzonitrile (0.6 g, 3.31 mmol) and Palladium 10% on Carbon (60.0 mg) in Methanol (10 mL) was hydrogenated in H₂ (Balloon) at 50° C. for 2 h. The mixture was filtered over Celite to remove Pd/C, and the solvent was evaporated to give the desired product 3,4-diamino-5-fluoro-benzonitrile (500 mg, 3.31 mmol, 99.86% yield) as a colorless solid. LC/MS(ESI⁺) [(M+H)⁺]: 151.8.

### Step 3:

To a solution of 3,4-diamino-5-fluoro-benzonitrile (0.5 g, 3.31 mmol), 3-Bromopropyne (472.25 mg, 3.97 mmol) in DMSO (6 mL) was added Potassium iodide (54.92 mg, 330.82 μmol) followed by DIPEA (855.10 mg, 6.62 mmol, 1.15 mL). After stirring at 120° C. for 4 hours, the reaction mixture was cooled down to RT, poured on to water (15 mL) and extracted with ethyl acetate (3×25 mL). The combined organic layers were washed with water (30 mL) and saturated aqueous sodium chloride (30 mL), dried over sodium sulfate and concentrated in vacuo. The crude product was purified by flash column chromatography on silica gel (0-30% ethyl acetate/heptane) to obtain 4-amino-3-fluoro-5-(prop-2-ynylamino) benzonitrile (0.3 g, 1.59 mmol, 47.93% yield) as a white solid. LC/MS(ESI⁺) [(M+H)⁺]: 189.8.

### Step 4:

To a solution of 4-amino-3-fluoro-5-(prop-2-ynylamino)benzonitrile (0.3 g, 1.59 mmol) and tert-butyl 11-ethyl-2-formyl-1,9-diazatricyclo[6.3.1.04,12]dodeca-2,4(12), 5,7-tetraene-9-carboxylate (498.51 mg, 1.59 mmol) in EtOH/H₂O (8 mL/4 mL) was added disodium hydrosulfite (828.27 mg, 4.76 mmol), the mixture was heated at reflux for 18 hours. After completion of the reaction, the mixture was concentrated in vacuo and the residue was extracted with EtOAc (2×50 mL). The combined organic extracts were washed with brine (40 mL), dried over sodium sulfate and evaporated to give the crude product. The crude product was purified by flash column chromatography using 15-30% EtOAc in hexane to afford title product tert-butyl 2-(6-cyano-4-fluoro-1-prop-2-ynyl-benzimidazol-2-yl)-11-ethyl-1,9-diazatricyclo[6.3.1.04,12]dodeca-2,4(12), 5,7-tetraene-9-carboxylate (0.22 g, 454.98 μmol, 28.69% yield) as a bluish white solid. LC/MS(ESI⁺) [(M+H)⁺]: 483.8.

### Step 5:

To a stirred solution tert-butyl 2-(6-cyano-4-fluoro-1-prop-2-ynyl-benzimidazol-2-yl)-11-ethyl-1,9-diazatricyclo[6.3.1.04,12]dodeca-2,4(12), 5,7-tetraene-9-carboxylate (220 mg, 454.98 μmol) in MeOH (2 mL), was added 4M HCl in dioxane (5 mL) and the reaction mixture was stirred at RT for 0.5 hour. The reaction mixture was evaporated to afford the crude product 2-(11-ethyl-1,9-diazatricyclo[6.3.1.04,12]dodeca-2,4(12), 5,7-tetraen-2-yl)-7-fluoro-3-prop-2-ynyl-benzimidazole-5-carbonitrile (190 mg, 452.51 μmol, 99.46% yield, HC) as a brown solid. LC/MS (ESI⁺) [(M+H)⁺]: 383.8.

### Step 6:

To a solution of 2-(11-ethyl-1,9-diazatricyclo[6.3.1.04,12]dodeca-2,4(12), 5,7-tetraen-2-yl)-7-fluoro-3-prop-2-ynyl-benzimidazole-5-carbonitrile (190 mg, 452.51 μmol, HC), 3-Bromo-1-propanol (314.47 mg, 2.26 mmol, 197.78 μL) in Acetonitrile, (3.0 mL) was added N,N-diisopropylethylamine (292.41 mg, 2.26 mmol, 394.09 μL). The resulting mixture was heated at 130° C. in a sealed tube for 18 h. The reaction was allowed to cool to RT and concentrated in vacuo. The crude material was purified by flash column chromatography on silica gel (5-50% ethyl acetate/heptane) to obtain 2-[11-ethyl-9-(3-hydroxypropyl)-1,9-diazatricyclo[6.3.1.04,12]dodeca-2,4(12), 5,7-tetraen-2-yl]-7-fluoro-3-prop-2-ynyl-benzimidazole-5-carbonitrile (70 mg, 158.55 μmol, 35.04% yield) as a pale-yellow oil. LC/MS(ESI⁺) [(M+H)⁺]: 441.7.

### Step 7:

To a solution of 2-[11-ethyl-9-(3-hydroxypropyl)-1,9-diazatricyclo[6.3.1.04,12]dodeca-2,4(12), 5,7-tetraen-2-yl]-7-fluoro-3-prop-2-ynyl-benzimidazole-5-carbonitrile (70 mg, 158.55 μmol) in MeOH (2 mL), water (2 mL) was added KOH (71.17 mg, 1.27 mmol). The mixture was stirred at 100° C. for 18 h. The reaction mixture was cooled down to RT, concentrated in vacuo and taken up in water (2 mL), acidified with 2N aqueous hydrochloric acid to pH about 2-3 and extracted with MeOH/CH₂Cl₂ (2×20 mL). The combined organic layer was dried over anhydrous sodium sulphate and filtered. The filtrate evaporated under vacuum to afford the product 2-[11-ethyl-9-(3-hydroxypropyl)-1,9-diazatricyclo[6.3.1.04,12]dodeca-2,4(12), 5,7-tetraen-2-yl]-7-fluoro-3-prop-2-ynyl-benzimidazole-5-carboxylic acid (35 mg, 76.00 μmol, 47.94% yield) as a pale oil. LC/MS(ESI⁺) [(M+H)⁺]: 460.7.

### Step 8:

To a solution of 2-[11-ethyl-9-(3-hydroxypropyl)-1,9-diazatricyclo[6.3.1.04,12]dodeca-2,4(12), 5,7-tetraen-2-yl]-7-fluoro-3-prop-2-ynyl-benzimidazole-5-carboxylic acid (35 mg, 76.00 μmol) and tert-butyl N-[(1R,4R,7R)-2-azabicyclo[2.2.1]heptan-7-yl]carbamate (16.13 mg, 76.00 μmol) in CH₂Cl₂ (5 mL) at RT was added HATU (37.57 mg, 98.81 μmol) and N,N-Diisopropylethylamine (29.47 mg, 228.01 μmol, 39.71 μL). The reaction mixture was stirred at RTfor 2 hours. After completion of the reaction, the reaction nmixture was quenched with H₂O (6 mL) and extracted with CH₂Cl₂(2*20 mL). The combined organic extracts were washed with brine (20 mL), dried over sodium sulfate and evaporated to give the crude product. The crude product was purified by flash column chromatography on silica gel using 2-20% MeOH in CH₂Cl₂ to afford title product tert-butyl N-[(1R,4R,7R)-2-[2-[11-ethyl-9-(3-hydroxypropyl)-1,9-diazatricyclo[6.3.1.04,12]dodeca-2,4(12), 5,7-tetraen-2-yl]-7-fluoro-3-prop-2-ynyl-benzimidazole-5-carbonyl]-2-azabicyclo[2.2.1]heptan-7-yl]carbamate (25 mg, 38.18 μmol, 50.24% yield) as a bluish white solid. LC/MS(ESI⁺) [(M+H)⁺]: 654.8.

### Step 9:

To a stirred solution tert-butyl N-[(1R,4R,7R)-2-[2-[11-ethyl-9-(3-hydroxypropyl)-1,9-diazatricyclo[6.3.1.04,12]dodeca-2,4(12), 5,7-tetraen-2-yl]-7-fluoro-3-prop-2-ynyl-benzimidazole-5-carbonyl]-2-azabicyclo[2.2.1]heptan-7-yl]carbamate (25.00 mg, 38.18 μmol) in MeOH (1 mL), was added 4M HCl in dioxane (3 mL) and the reaction mixture was stirred at RT for 0.5 hour. The reaction mixture was evaporated to afford the crude product and then prep-HPLC to give [(1R,4R,7R)-7-amino-2-azabicyclo[2.2.1]heptan-2-yl]-[2-[11-ethyl-9-(3-hydroxypropyl)-1,9-diazatricyclo[6.3.1.04,12]dodeca-2,4(12), 5,7-tetraen-2-yl]-7-fluoro-3-prop-2-ynyl-benzimidazol-5-yl]methanone (12.5 mg, 22.54 μmol, 59.03% yield) as a white solid. LC/MS(ESI⁺) [(M+H)⁺]: 554.8.

### Example 102 Synthesis report of ((1R,4R,7R)-7-amino-2-azabicyclo[2.2.1]heptan-2-yl)(2-(3-ethyl-2,3-dihydro-1H-pyrrolo[1,2,3-de]quinoxalin-5-yl)-7-methoxy-1-methyl-1H-benzo[d]imidazol-5-yl)methanone

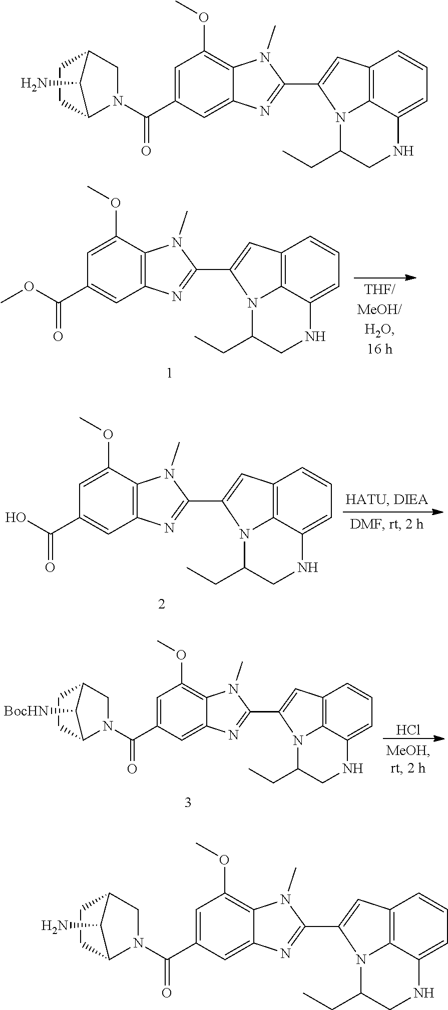 [SMILES: CCC1CNc2cccc3cc(-c4nc5cc(C(=O)N6C[C@H]7CC[C@@H]6[C@@H]7N)cc(OC)c5n4C)n1c23 ; CCC1CNc2cccc3cc(-c4nc5cc(C(=O)OC)cc(OC)c5n4C)n1c23 ; CCC1CNc2cccc3cc(-c4nc5cc(C(=O)O)cc(OC)c5n4C)n1c23 ; CCC1CNc2cccc3cc(-c4nc5cc(C(=O)N6C[C@H]7CC[C@@H]6[C@@H]7NC(=O)OC(C)(C)C)cc(OC)c5n4C)n1c23 ; Cl ; CCC1CNc2cccc3cc(-c4nc5cc(C(=O)N6C[C@H]7CC[C@@H]6[C@@H]7N)cc(OC)c5n4C)n1c23]

### Step 1:

To a stirred solution of methyl 2-(11-ethyl-1,9-diazatricyclo[6.3.1.0^{4,12}]dodeca-2,4(12), 5,7-tetraen-2-yl)-7-methoxy-1-methyl-benzimidazole-5-carboxylate (100 mg, 247.24 μmol) in THF (2.0 mL), MeOH (1.0 mL) was added LiOH aqueous solution (1.0 M, 0.74 mL). The mixture was stirred at RT for 16 hours, acidified to pH 5-6 with 3M hydrochloric acid aqueous solution, and extracted with EA (20*3 mL). The organic phase was dried over anhydrous sodium sulfate and filtered. The filtrate was concentrated in vacuo to give 72-(11-ethyl-1,9-diazatricyclo[6.3.1.0^{4,12}]dodeca-2,4(12), 5,7-tetraen-2-yl)-7-methoxy-1-methyl-benzimidazole-5-carboxylic acid (90 mg, 230.51 μmol, 93.23% yield) as a white solid. LC/MS(ESI⁺) [(M+H)⁺]: 390.8.

### Step 2:

To a solution 2-(11-ethyl-1,9-diazatricyclo[6.3.1.0^{4,12}]dodeca-2,4(12), 5,7-tetraen-2-yl)-7-methoxy-1-methyl-benzimidazole-5-carboxylic acid (90 mg, 230.51 μmol) in DMF (2 mL), DIPEA (89.37 mg, 691.54 μmol, 120.45 μL), HATU (131.47 mg, 345.77 μmol), tert-butyl N-[(1R,4R,7R)-2-azabicyclo[2.2.1]heptan-7-yl]carbamate (48.94 mg, 230.51 μmol) was added into the mixture, the mixture was stirred at RT for 2 hours. LC-MS showed the starting material was consumed and the desired mass was detected, and diluted with EA and washed with brine, and dried over anhydrous sodium sulfate. After filtration and evaporation of the solvent in vacuo, and the residue was purified by flash column chromatography on silica gel(elution with DCM/MeOH=1:0-12:1) to give tert-butyl N-[(1R,4R,7R)-2-[2-(11-ethyl-1,9-diazatricyclo[6.3.1.0^{4,12}]dodeca-2,4,6,8(12)-tetraen-2-yl)-7-methoxy-1-methyl-benzimidazole-5-carbonyl]-2-azabicyclo[2.2.1]heptan-7-yl]carbamate (80 mg, 136.82 μmol, 59.35% yield) as a yellow solid. LC/MS(ESI⁺) [(M+H)⁺]: 584.7.

### Step 3:

To a solution of tert-butyl N-[(1R,4R,7R)-2-[2-(11-ethyl-1,9-diazatricyclo[6.3.1.0^{4,12}]dodeca-2,4,6,8(12)-tetraen-2-yl)-7-methoxy-1-methyl-benzimidazole-5-carbonyl]-2-azabicyclo[2.2.1]heptan-7-yl]carbamate (80 mg, 136.82 μmol) in MeOH (2 mL), added 4M HCl(dioxane) (2 mL). The mixture was stirred at RT for 2 h. The mixture was concentrated in vacuo, the reaction mixture was adjusted pH to 8 with saturated Na₂CO₃ solution, The mixture was extracted with DCM (30 mL*3). The organic layer was dried over anhydrous sodium sulfate. After filtration and evaporation of the solvent in vacuo, and the residue was purified by flash column chromatography on silica gel(elution with DCM/MeOH=1:0-10:1) to give [(1R,4R,7R)-7-amino-2-azabicyclo[2.2.1]heptan-2-yl]-[2-(11-ethyl-1,9-diazatricyclo[6.3.1.0^{4,12}]dodeca-2,4,6,8(12)-tetraen-2-yl)-7-methoxy-1-methyl-benzimidazol-5-yl]methanone (55 mg, 113.50 μmol, 82.95% yield) as a white solid. LC/MS(ESI⁺) [(M+H)⁺]: 484.7. ¹H NMR (400 MHz, DMSO-d₆) δ 7.51-7.31 (m, 1H), 7.00-6.89 (m, 3H), 6.85 (t, J=7.6 Hz, 1H), 6.36 (d, J=7.0 Hz, 1H), 6.05 (s, 1H), 5.22 (s, 1H), 4.21 (s, 3H), 3.99 (s, 3H), 3.85 (d, J=12.5 Hz, 1H), 3.59-3.48 (m, 3H), 3.23 (s, 1H), 3.08 (d, J=11.0 Hz, 1H), 2.29 (s, 1H), 1.97 (s, 2H), 1.56 (dd, J=12.4, 5.4 Hz, 2H), 1.51-1.41 (m, 1H), 1.32 (d, J=15.4 Hz, 1H), 1.25 (dd, J=9.6, 3.6 Hz, 2H), 0.67-0.56 (m, 3H).

### Example 103 Preparation of ((1R,4R,7R)-7-amino-2-azabicyclo[2.2.1]heptan-2-yl)(7-fluoro-2-(1-(3-hydroxypropyl)-3-isopropyl-2,3-dihydro-1H-pyrrolo[1,2,3-de]quinoxalin-5-yl)-1-methyl-1H-benzo[d]imidazol-5-yl)methanone

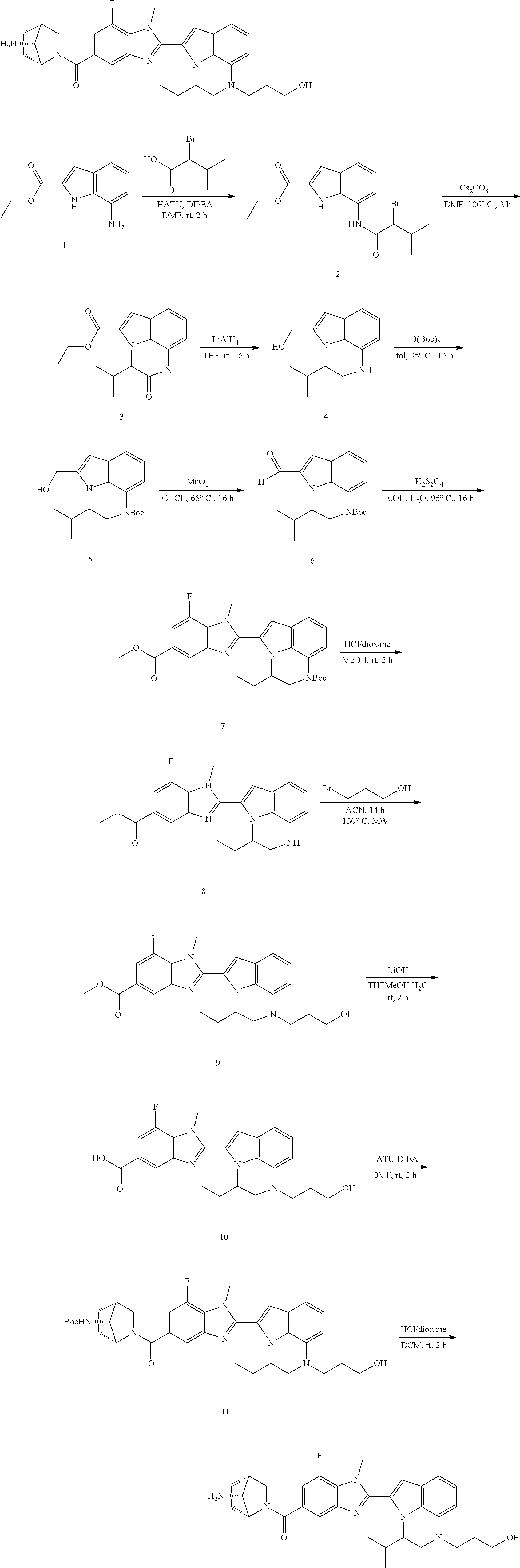

### Step 1:

To a solution of ethyl 7-amino-1H-indole-2-carboxylate (4 g, 19.59 mmol) and 2-bromo-3-methylbutanoic acid (3.55 g, 19.59 mmol) in DMF (60 mL) at RT was added DIPEA (7.59 g, 58.76 mmol, 10.23 mL) and HATU (11.17 g, 29.38 mmol). The reaction mixture was stirred at RT for 16 hours. LC-MS showed the starting material was consumed and the desired mass was detected, the reaction mixture was diluted with EA and washed with brine and dried over anhydrous sodium sulfate. After filtration and evaporation of the solvent in vacuo, and the residue was purified by flash column chromate graphy(elution with PE/EA=1:0-5:1) on silica gel to afford ethyl 7-(2-bromo-3-methylbutanamido)-1H-indole-2-carboxylate (5.1 g, 13.89 mmol, 70.90% yield) as a bluish white solid. LC/MS (ESI⁺) [(M+H)⁺]: 367.8.

### Step 2:

To a solution of ethyl 7-[(2-bromo-3-methyl-butanoyl)amino]-1H-indole-2-carboxylate (5.1 g, 13.89 mmol) in DMSO (50 mL) was added dicesium carbonate (13.57 g, 41.66 mmol) at RT. The reaction mixture was stirred at 106° C. for 2 h, cooled to RT, quenched with ice H₂O (20 mL) and extracted with ethyl acetate (100 mL×3). The organic phase was washed with brine (50 mL×4) and dried over anhydrous sodium sulfate and filtered. The filtrate was concentrated in vacuo, and the residue was purified by flash column chromatography on silica gel (PE/EA=1/0-2/1) to provide ethyl 3-isopropyl-2-oxo-2,3-dihydro-1H-pyrrolo[1,2,3-de]quinoxaline-5-carboxylate (3.1 g, 10.83 mmol, 77.96% yield) as a white solid. LC/MS (ESI⁺) [(M+H)⁺]: 286.8.

### Step 3:

To a solution of ethyl 11-isopropyl-10-oxo-1,9-diazatricyclo[6.3.1.0^{4,12}]dodeca-2,4,6,8(12)-tetraene-2-carboxylate (1.0 g, 3.49 mmol) in THF (20 mL) was added lithium aluminum hydride (473.9 mg, 13.97 mmol) at 0° C. in batches over 10 min. After 10 min, the bath was removed, and the solution was stirred at rt for 16 h. The reaction mixture was quenched with ice (0.5 g) and added 0.5 mL 15% saturated NaOH and 1.0 mL H₂O at 0° C. The solution was stirred at RT for 15 min, poured into a mixture of ethyl acetate (100 mL). The organic phase was washed with brine, dried over anhydrous sodium sulfate. After filtration and evaporation of the solvent in vacuo, and the residue was purified by flash column chromatography on silica gel (PE/EA=1/0-1/1) to provide (11-isopropyl-1,9-diazatricyclo[6.3.1.0^{4,12}]dodeca-2,4,6,8(12)-tetraen-2-yl)methanol as a white solid. LC/MS(ESI⁺)[(M+H)⁺]: 230.8.

### Step 4:

To a solution of (11-isopropyl-1,9-diazatricyclo[6.3.1.0^{4,12}]dodeca-2,4,6,8(12)-tetraen-2-yl)methanol (300 mg, 1.30 mmol) in toluene (6 mL) was added di-tert-butyl dicarbonate (568.6 mg, 2.61 mmol, 597.89 μL). The mixture was stirred at 95° C. for 16 h. The reaction mixture was cooled to rt, diluted with EA and washed with brine and dried over anhydrous sodium sulfate. After filtration and evaporation of the solvent in vacuo, and the residue was purified by flash column chromatography on silica gel (elution with PE/EA=1:0-5:1) to afford tert-butyl 5-(hydroxymethyl)-3-isopropyl-2,3-dihydro-1H-pyrrolo[1,2,3-de]quinoxaline-1-carboxylate (320 mg, 968.46 μmol, 74.35% yield) as a brown solid. LC/MS (ESI⁺) [(M+H)⁺]: 330.8.

### Step 5:

The mixture of tert-butyl 2-(hydroxymethyl)-11-isopropyl-1,9-diazatricyclo[6.3.1.0^{4,12}]dodeca-2,4,6,8(12)-tetraene-9-carboxylate (320 mg, 968.46 μmol) and manganese dioxide (344.6 mg, 3.87 mmol) in CHCl₃ (6 mL) was stirred at 66° C. for 16 hours, cooled to rt and filtered. The filtrate was concentrated in vacuo to afford tert-butyl 2-formyl-11-isopropyl-1,9-diazatricyclo[6.3.1.0^{4,12}]dodeca-2,4,6,8(12)-tetraene-9-carboxylate (310 mg, 943.96 μmol, 97.47% yield). LC/MS (ESI⁺) [(M+H)⁺]: 328.8.

### Step 6:

A mixture of tert-butyl 2-formyl-11-isopropyl-1,9-diazatricyclo[6.3.1.0^{4,12}]dodeca-2,4,6,8(12)-tetraene-9-carboxylate (310 mg, 943.96 μmol), methyl 3-fluoro-4-(methylamino)-5-nitrobenzoate (236.93 mg, 1.04 mmol) and sodium dithionite (986.11 mg, 5.66 mmol) in mixed solvent of EtOH (10 mL) and H₂O (10 mL) was stirred at 96° C. for 16 hours, cooled to RT and concentrated in vacuo. The residue was extracted with DCM (10 mL*3), and the organic phase was dried over anhydrous sodium sulfate and filtered. The filtrate was concentrated in vacuo and the residue was purified by flash column chromatography(elution with PE/EA=1:0-10:1) on silica gel to afford tert-butyl 5-(7-fluoro-5-(methoxycarbonyl)-1-methyl-1H-benzo[d]imidazol-2-yl)-3-isopropyl-2,3-dihydro-1H-pyrrolo[1,2,3-de]quinoxaline-1-carboxylate (300 mg, 592.22 μmol, 62.74% yield) as a white solid. LC/MS (ESI⁺) [(M+H)⁺]: 506.8.

### Step 7:

To a solution of tert-butyl 2-(7-fluoro-5-methoxycarbonyl-1-methyl-benzimidazol-2-yl)-11-isopropyl-1,9-diazatricyclo[6.3.1.0^{4,12}]dodeca-2,4,6,8(12)-tetraene-9-carboxylate (300 mg, 592.22 μmol) in MeOH (3 mL), added 4M HCl(dioxane) (3 mL). The mixture was stirred at RT for 2 h. The mixture was concentrated in vacuo, the reaction mixture was adjusted pH to 8 with saturated Na₂CO₃ solution. The mixture was extracted with DCM (30 mL*3). The organic layer was dried over anhydrous sodium sulfate. After filtration and evaporation of the solvent in vacuo, and the residue was purified by silica gel flash column chromatography(elution with DCM/MeOH=1:0-20:1) to give methyl 7-fluoro-2-(11-isopropyl-1,9-diazatricyclo[6.3.1.0^{4,12}]dodeca-2,4,6,8(12)-tetraen-2-yl)-1-methyl-benzimidazole-5-carboxylate (220 mg, 541.27 μmol, 91.40% yield) as a white solid. LC/MS(ESI⁺) [(M+H)⁺]: 406.8.

### Step 8:

To a solution of methyl 7-fluoro-2-(11-isopropyl-1,9-diazatricyclo[6.3.1.0^{4,12}]dodeca-2,4,6,8(12)-tetraen-2-yl)-1-methyl-benzimidazole-5-carboxylate (100 mg, 246.03 μmol) in anhydrous ACN (3 mL) were added DIPEA (159.0 mg, 1.23 mmol, 214.27 μL) and 3-bromopropan-1-ol (171.0 mg, 1.23 mmol, 107.54 μL) at RT. The reaction mixture was heated at 130° C. for 14 h by microwave, cooled to RT and concentrated in vacuo, and the residue was purified by flash column chromatography on silica gel (elution with DCM/MeOH=1:0-20:1) to provide methyl 7-fluoro-2-(1-(3-hydroxypropyl)-3-isopropyl-2,3-dihydro-1H-pyrrolo[1,2,3-de]quinoxalin-5-yl)-1-methyl-1H-benzo[d]imidazole-5-carboxylate (25 mg, 53.82 μmol, 21.87% yield) as a yellow solid. LC/MS(ESI⁺) [(M+H)⁺]: 464.8.

### Step 9:

To a stirred solution of methyl 7-fluoro-2-[9-(3-hydroxypropyl)-11-isopropyl-1,9-diazatricyclo[6.3.1.0^{4,12}]dodeca-2,4,6,8(12)-tetraen-2-yl]-1-methyl-benzimidazole-5-carboxylate (25 mg, 53.82 μmol) in THF (1.00 mL), MeOH (0.5 mL) was added LiOH aqueous solution(1.0 M, 0.5 mL). The mixture was stirred at RT for 2 hours, acidified to pH 5-6 with 3 M hydrochloric acid aqueous solution, and extracted with EA(20*3 mL). The organic phase was dried over anhydrous sodium sulfate and filtered. The filtrate was concentrated in vacuo to give 7-fluoro-2-[9-(3-hydroxypropyl)-11-isopropyl-1,9-diazatricyclo[6.3.1.0^{4,12}]dodeca-2,4,6,8(12)-tetraen-2-yl]-1-methyl-benzimidazole-5-carboxylic acid (20 mg, 44.39 μmol, 82.49% yield) as a white solid, LC/MS (ESI⁺) [(M+H)⁺]: 450.8.

### Step 10:

To a solution 7-fluoro-2-[9-(3-hydroxypropyl)-11-isopropyl-1,9-diazatricyclo[6.3.1.0^{4,12}]dodeca-2,4,6,8(12)-tetraen-2-yl]-1-methyl-benzimidazole-5-carboxylic acid (20 mg, 44.39 μmol) in DMF (2 mL), DIPEA (17.21 mg, 133.18 μmol, 23.20 μL), HATU (25.3 mg, 66.59 μmol), tert-butyl N-[(1R,4R,7R)-2-azabicyclo[2.2.1]heptan-7-yl]carbamate (PhannaBlock) (9.4 mg, 44.39 μmol) was added into the mixture, the mixture was stirred at RT for 2 hours. LC-MS showed the starting material was consumed and the desired mass was detected, and diluted with EA and washed with brine, and dried over anhydrous sodium sulfate. After filtration and evaporation of the solvent in vacuo, and the residue was purified by flash column chromatography on silica gel(elution with DCM/MeOH=1:0-15:1) to give tert-butyl ((1R,4R,7R)-2-(7-fluoro-2-(1-(3-hydroxypropyl)-3-isopropyl-2,3-dihydro-1H-pyrrolo[1,2,3-de]quinoxalin-5-yl)-1-methyl-1H-benzo[d]imidazole-5-carbonyl)-2-azabicyclo[2.2.1]heptan-7-yl)carbamate (15 mg, 23.26 μmol, 52.40% yield) as a yellow solid. LC/MS(ESI⁺) [(M+H)⁺]: 644.8.

### Step 11:

To a solution of tert-butyl ((1R,4R,7R)-2-(7-fluoro-2-(1-(3-hydroxypropyl)-3-isopropyl-2,3-dihydro-1H-pyrrolo[1,2,3-de]quinoxalin-5-yl)-1-methyl-1H-benzo[d]imidazole-5-carbonyl)-2-azabicyclo[2.2.1]heptan-7-yl)carbamate (15 mg, 23.26 μmol) in MeOH (2 mL), added 4M HCl(dioxane) (2 mL). The mixture was stirred at RT for 2 h. The mixture was concentrated in vacuo, the reaction mixture was adjusted pH to 8 with saturated Na₂CO₃ solution, The mixture was extracted with DCM (30 mL*3). The organic layer was dried over anhydrous sodium sulfate. After filtration and evaporation of the solvent in vacuo, and the residue was purified by flash column chromatography on silica gel (elution with DCM/MeOH=1:0-10:1) to give [(1R,4R,7R)-7-amino-2-azabicyclo[2.2.1]heptan-2-yl]-[7-fluoro-2-[9-(3-hydroxypropyl)-11-isopropyl-1,9-diazatricyclo[6.3.1.0^{4,12}]dodeca-2,4,6,8(12)-tetraen-2-yl]-1-methyl-benzimidazol-5-yl]methanone (8.6 mg, 15.79 μmol, 67.87% yield) as a white solid. LC/MS(ESI⁺) [(M+H)⁺]: 544.8.

### Example 104 ((1R,4R,7R)-7-amino-2-azabicyclo[2.2.1]heptan-2-yl)(7-fluoro-2-(3-isopropyl-2,3-dihydro-1H-pyrrolo[1,2,3-de]quinoxalin-5-yl)-1-methyl-1H-benzo[d]imidazol-5-yl)methanone

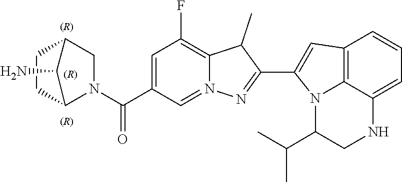

Prepared in analogous manner as for Example 103. LC/MS(ESI⁺) [(M+H)⁺]:486.7. ¹H NMR (400 MHz, DMSO-d₆) δ 7.66 (dd, J=45.7, 4.3 Hz, 1H), 7.24 (dd, J=30.3, 12.0 Hz, 1H), 6.99 (s, 1H), 6.94 (d, J=7.9 Hz, 1H), 6.87 (t, J=7.6 Hz, 1H), 6.39 (d, J=7.1 Hz, 1H), 6.07 (s, 1H), 5.10 (d, J=7.1 Hz, 1H), 4.17 (s, 3H), 3.72 (dd, J=27.9, 12.4 Hz, 2H), 3.51 (dd, J=12.0, 3.0 Hz, 2H), 3.23 (s, 2H), 3.11-3.04 (m, 1H), 2.21 (d, J=24.7 Hz, 1H), 2.03-1.82 (m, 3H), 1.75 (d, J=8.4 Hz, 1H), 1.42 (dd, J=29.7, 19.9 Hz, 1H), 1.28-1.21 (m, 1H), 0.78 (d, J=6.7 Hz, 3H), 0.43-0.34 (m, 3H).

### Example 105 ((1R,4R,7R)-7-amino-2-azabicyclo[2.2.1]heptan-2-yl)(7-fluoro-2-((R)-3-isopropyl-2,3-dihydro-1H-pyrrolo[1,2,3-de]quinoxalin-5-yl)-1-methyl-1H-benzo[d]imidazol-5-yl)methanone and Example 106 ((1R,4R,7R)-7-amino-2-azabicyclo[2.2.1]heptan-2-yl)(7-fluoro-2-((S)-3-isopropyl-2,3-dihydro-1H-pyrrolo[1,2,3-de]quinoxalin-5-yl)-1-methyl-1H-benzo[d]imidazol-5-yl)methanone

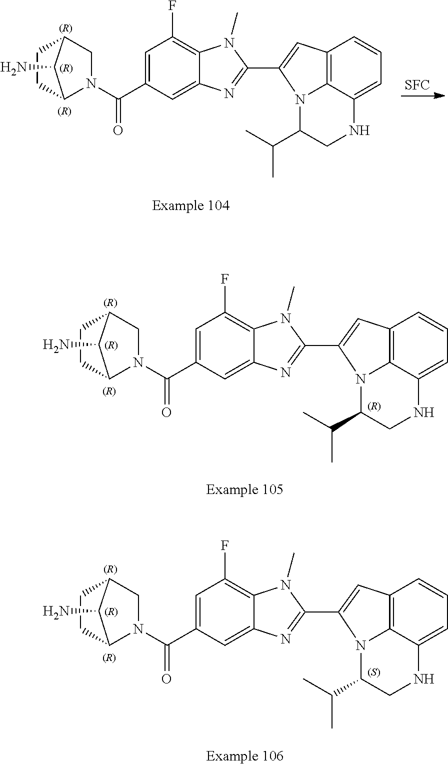

((1R,4R,7R)-7-amino-2-azabicyclo[2.2.1]heptan-2-yl)(7-fluoro-2-(3-isopropyl-2,3-dihydro-1H-pyrrolo[1,2,3-de]quinoxalin-5-yl)-1-methyl-1H-benzo[d]imidazol-5-yl)methanone (302 mg, 0.62 mmol) was chirally separated by SFC with mobile phase (CO2/MeOH[0.2% NH3(7M in MeOH)]=60/40) (AD 20*250 mm, 10 um (Daicel) (Flow Rate: 100 g/min) to give synthetic Example 105 (80 mg, 16.4%) as an off-white solid (LCMS(ESI⁺) [(M+H)⁺]: 486.7, H NMR (400 MHz, DMSO-d₆) δ 7.61 (s, 1H), 7.35-7.17 (m, 1H), 6.99 (d, J=2.9 Hz, 1H), 6.93 (d, J=8.0 Hz, 1H), 6.87 (t, J=7.6 Hz, 1H), 6.38 (d, J=7.1 Hz, 1H), 6.09 (d, J=3.1 Hz, 1H), 5.10 (d, J=8.1 Hz, 1H), 4.16 (d, J=3.2 Hz, 3H), 3.72 (d, J=15.5 Hz, 1H), 3.69-3.59 (m, 1H), 3.54-3.48 (m, 2H), 3.19 (s, 1H), 3.06 (t, J=9.4 Hz, 1H), 2.20 (d, J=3.9 Hz, 1H), 2.04-1.82 (m, 4H), 1.76-1.65 (m, 1H), 1.46-1.33 (m, 1H), 1.26-1.21 (m, 1H), 0.78 (d, J=6.6 Hz, 3H), 0.38 (dd, J=7.0, 4.1 Hz, 3H) and Example 106 (100 mg, 20.5%) as off-white solid (LCMS(ESI⁺) [(M+H)⁺]: 486.7, ¹H NMR (400 MHz, DMSO-d₆) δ 7.38 (s, 1H), 7.10-6.96 (m, 1H), 6.78 (s, 1H), 6.72 (d, J=7.9 Hz, 1H), 6.66 (t, J=7.6 Hz, 1H), 6.17 (d, J=7.1 Hz, 1H), 5.88 (d, J=3.1 Hz, 1H), 4.92-4.85 (m, 1H), 3.95 (d, J=3.0 Hz, 3H), 3.51-3.41 (m, 2H), 3.34-3.25 (m, 2H), 2.97 (s, 1H), 2.89-2.76 (m, 1H), 1.99 (s, 1H), 1.79-1.63 (m, 3H), 1.56-1.49 (m, 1H), 1.26-1.14 (m, 1H), 1.02 (d, J=3.4 Hz, 1H), 0.57 (dd, J=6.8, 2.0 Hz, 3H), 0.17 (d, J=7.2 Hz, 3H).

Based on the co-cystallization results of Example 71, it is believed that Example 106, which is more potent than Example 105 in the PAD4 biochemical assay, has the S configuration.

### Example 107 Preparation of ((1R,4R,7R)-7-amino-2-azabicyclo[2.2.1]heptan-2-yl)(7-fluoro-2-(3-isopropyl-1-methyl-2,3-dihydro-1H-pyrrolo[1,2,3-de]quinoxalin-5-yl)-1-methyl-1H-benzo[d]imidazol-5-yl)methanone

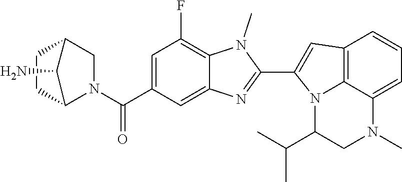 [SMILES: CC(C)C1CN(C)c2cccc3cc(-c4nc5cc(C(=O)N6C[C@H]7CC[C@@H]6[C@@H]7N)cc(F)c5n4C)n1c23]

Prepared in analogous manner as for Example 103. LC/MS(ESI⁺) [(M+H)⁺]: 500.8.

### Example 108 ((1R,4R,7R)-7-amino-2-azabicyclo[2.2.1]heptan-2-yl)(2-(3-cyclopropyl-2,3-dihydro-1H-pyrrolo[1,2,3-de]quinoxalin-5-yl)-7-fluoro-1-methyl-1H-benzo[d]imidazol-5-yl)methanone

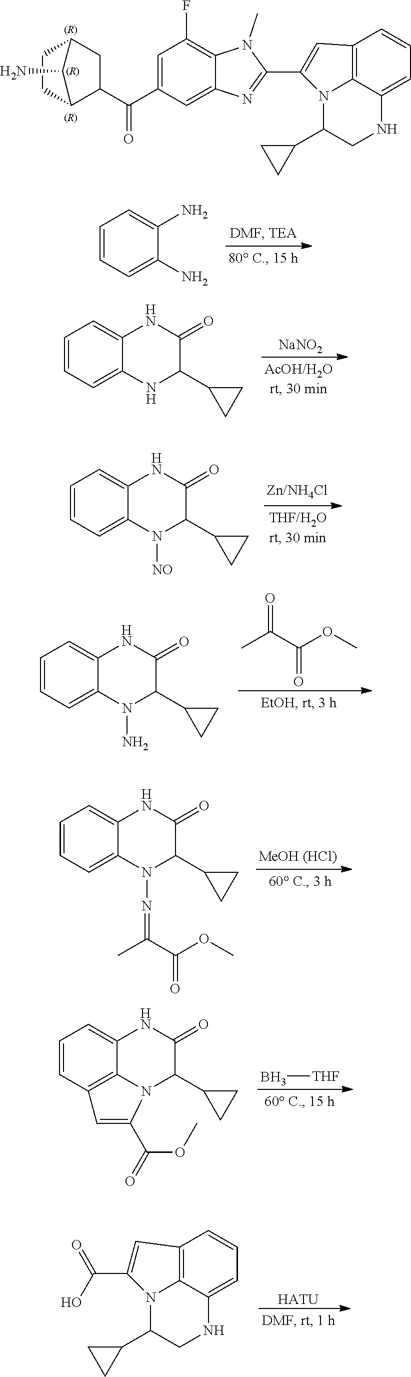

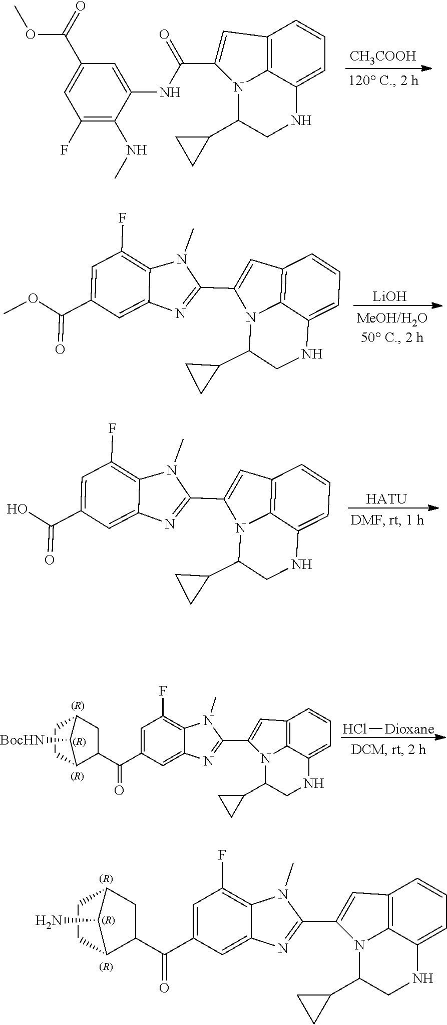

### Step 1:

A mixture of methyl 2-bromo-2-cyclopropyl-acetate (2.0 g, 10.36 mmol), benzene-1,2-diamine (1.34 g, 12.43 mmol, 1.31 mL), TEA (2.10 g, 20.72 mmol, 2.89 mL) was dissolved in DMF (20 mL). The resulting mixture was stirred at 80° C. for 15 h. The crude material was purified by flash column chromatography on silica gel (eluting with H₂O/CH₃CN from 0 to 100%) to give 3-cyclopropyl-3,4-dihydro-1H-quinoxalin-2-one (1.5 g, 7.97 mmol, 76.92% yield) as a white solid. LC/MS(ESI⁺) [(M+H)⁺]: 188.8.

### Step 2:

A mixture of 3-cyclopropyl-3,4-dihydro-1H-quinoxalin-2-one (1.5 g, 7.97 mmol), sodium nitrite (659.8 mg, 9.56 mmol, 304.36 μL) was dissolved in CH₃COOH (8 mL). The resulting mixture was stirred at rt for 30 min. Filtered to give 3-cyclopropyl-4-nitroso-1,3-dihydroquinoxalin-2-one (1.2 g, 5.52 mmol, 69.32% yield) as a yellow solid. LC/MS(ESI⁺) [(M+H)⁺]: 217.8.

### Step 3:

A mixture of 3-cyclopropyl-4-nitroso-1,3-dihydroquinoxalin-2-one (1.2 g, 5.52 mmol), ammonium hydrochloride (2.07 g, 38.67 mmol) and zinc (1.81 g, 27.62 mmol, 252.96 μL) were dissolved in THF:H₂O=1:1 (12 mL). The resulting mixture was stirred at RT for 2 h. The reaction was diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give 4-amino-3-cyclopropyl-1,3-dihydroquinoxalin-2-one (800.0 mg, 3.94 mmol, 71.25% yield) as a yellow solid. LC/MS(ESI⁺) [(M+H)⁺]: 203.8.

### Step 4:

A mixture of 4-amino-3-cyclopropyl-1,3-dihydroquinoxalin-2-one (800 mg, 3.94 mmol), methyl 2-oxopropanoate (602.8 mg, 5.90 mmol, 533.42 μL) was dissolved in ethanol (8 mL). The resulting mixture was stirred at rt for 3 h, then concentrated in vacuo to give methyl (2E)-2-[(2-cyclopropyl-3-oxo-2,4-dihydroquinoxalin-1-yl)imino]propanoate (1.0 g, 3.48 mmol, 88.42% yield) as a yellow solid. LC/MS(ESI⁺) [(M+H)⁺]: 287.8

### Step 5:

A mixture of methyl (2E)-2-[(2-cyclopropyl-3-oxo-2,4-dihydroquinoxalin-1-yl)imino]propanoate (1.0 g, 3.48 mmol), HCl (4 M in MeOH) 2 mL was dissolved in methanol (8 mL). The resulting mixture was stirred at 60° C. for 3 h. Concentrated in vacuo, the crude material was purified by flash column chromatogrpahy on silica gel (eluting with EA/PE from 0 to 100%) to give methyl 11-cyclopropyl-10-oxo-1,9-diazatricyclo[6.3.1.04,12]dodeca-2,4(12), 5,7-tetraene-2-carboxylate (600.0 mg, 2.22 mmol, 63.78% yield) as a yellow solid. LC/MS(ESI⁺) [(M+H)⁺]: 270.8

### Step 6:

To a solution of a mixture of methyl 11-cyclopropyl-10-oxo-1,9-diazatricyclo[6.3.1.04,12]dodeca-2,4(12), 5,7-tetraene-2-carboxylate (300.0 mg, 1.11 mmol) in THF (3 mL), was added BH₃-THF (2.5M in THF) 1 mL. The resulting mixture was stirred at 60° C. for 15 h. diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give methyl 11-cyclopropyl-1,9-diazatricyclo[6.3.1.04,12]dodeca-2,4(12), 5,7-tetraene-2-carboxylate (120.0 mg, 468.20 μmol, 42.18% yield) as yellow liquid. LC/MS(ESI⁺) [(M+H)⁺]: 256.8

### Step 7:

A mixture of methyl 11-cyclopropyl-1,9-diazatricyclo[6.3.1.04,12]dodeca-2,4(12), 5,7-tetraene-2-carboxylate (120.0 mg, 468.20 μmol), lithium hydroxide hydrate (39.3 mg, 936.41 μmol, 26.02 μL) was dissolved in MeOH:H₂O=1:1 (3 mL). The resulting mixture was stirred at 50° C. for 2 h. The reaction mixture was acidified with 3 mol/L hydrochloric acid. The reaction was diluted with EtOAc (50 mL) and washed with water (25 mL). The organic layer was dried over sodium sulfate, filtered and concentrated in vacuo. The crude material was purified by flash column chromatogrpahy on silica gel (eluting with EA/PE from 0 to 100%) to give 11-cyclopropyl-1,9-diazatricyclo[6.3.1.04,12]dodeca-2,4(12), 5,7-tetraene-2-carboxylic acid (100.0 mg, 412.76 μmol, 88.16% yield) as yellow liquid. LC/MS(ESI⁺) [(M+H)⁺]: 242.8

### Step 8:

A mixture of methyl 3-amino-5-fluoro-4-(methylamino)benzoate (89.9 mg, 454.03 μmol), 11-cyclopropyl-1,9-diazatricyclo[6.3.1.04,12]dodeca-2,4(12), 5,7-tetraene-2-carboxylic acid (100.0 mg, 412.76 μmol), DIPEA (106.7 mg, 825.52 μmol, 143.79 μL) and HATU (235.4 mg, 619.14 μmol) was dissolved in DMF (2 mL). The resulting mixture was stirred at RT for 1 h. The reaction was diluted with EtOAc (50 mL) and washed with water (25 mL). The organic layer was dried over sodium sulfate, filtered and concentrated in vacuo. The crude material was purified by flash column chromatogrpahy on silica gel (eluting with EA/PE from 0 to 100%) to give methyl 3-[(11-cyclopropyl-1,9-diazatricyclo[6.3.1.04,12]dodeca-2,4(12), 5,7-tetraene-2-carbonyl)amino]-5-fluoro-4-(methylamino)benzoate (80.0 mg, 189.37 μmol, 45.88% yield) as a yellow solid. LC/MS(ESI⁺) [(M+H)]: 422.8

### Step 9:

A mixture of methyl 3-[(11-cyclopropyl-1,9-diazatricyclo[6.3.1.04,12]dodeca-2,4(12), 5,7-tetraene-2-carbonyl)amino]-5-fluoro-4-(methylamino)benzoate (80.0 mg, 189.37 μmol) was dissolved in acetic acid (2 mL). It was stirred at 120° C. for 2 h. The reaction was concentrated in vacuo. The crude material was purified by flash column chromatogrpahy on silica gel (eluting with EA/PE from 0 to 100%) to give methyl 2-(11-cyclopropyl-1,9-diazatricyclo[6.3.1.04,12]dodeca-2,4(12), 5,7-tetraen-2-yl)-7-fluoro-1-methyl-benzimidazole-5-carboxylate (60.0 mg, 148.35 μmol, 78.34% yield) as a yellow solid. LC/MS(ESI⁺) [(M+H)⁺]: 404.8

### Step 10:

A mixture of methyl 2-(11-cyclopropyl-1,9-diazatricyclo[6.3.1.04,12]dodeca-2,4(12), 5,7-tetraen-2-yl)-7-fluoro-1-methyl-benzimidazole-5-carboxylate (60.0 mg, 148.35 μmol) and lithium hydroxide hydrate (12.4 mg, 296.71 μmol, 8.24 μL) was dissolved in MeOH/H₂O=1/1 (2 mL). The mixture was stirred at 50° C. for 2 h. The mixture was acidified with 3 mol/L hydrochloric acid. The reaction was diluted with EtOAc (50 mL) and washed with water (25 mL). The organic layer was dried over sodium sulfate, filtered, and concentrated in vacuo. The crude material was purified by flash column chromatogrpahy on silica gel (eluting with EA/PE from 0 to 100%) to give 2-(11-cyclopropyl-1,9-diazatricyclo[6.3.1.04,12]dodeca-2,4(12), 5,7-tetraen-2-yl)-7-fluoro-1-methyl-benzimidazole-5-carboxylic acid (40.0 mg, 102.46 μmol, 69.06% yield) as yellow liquid. LC/MS(ESI⁺) [(M+H)⁺]: 390.8.

### Step 11:

A mixture of 2-(11-cyclopropyl-1,9-diazatricyclo[6.3.1.04,12]dodeca-2,4(12), 5,7-tetraen-2-yl)-7-fluoro-1-methyl-benzimidazole-5-carboxylic acid (40.0 mg, 102.46 μmol), tert-butyl N-[(1R,4R,7R)-2-azabicyclo[2.2.1]heptan-7-yl]carbamate (PharmaBlock) (26.1 mg, 122.95 μmol), DIPEA (26.5 mg, 204.91 μmol, 35.69 μL) and HATU (58.4 mg, 153.68 μmol) was dissolved in DMF (1.5 mL). The resulting mixture was stirred at rt for 1 h. Desired signal was found by LC/MS. The reaction was diluted with EtOAc (50 mL) and washed with water (25 mL). The organic layer was dried over sodium sulfate, filtered, and concentrated in vacuo. The crude material was purified by flash column chromatography on silica gel (eluting with EA/PE from 0 to 100%) to give tert-butyl N-[(1R,4R,7R)-2-[2-(11-cyclopropyl-1,9-diazatricyclo[6.3.1.04,12]dodeca-2,4(12), 5,7-tetraen-2-yl)-7-fluoro-1-methyl-benzimidazole-5-carbonyl]-2-azabicyclo[2.2.1]heptan-7-yl]carbamate (30 mg, 51.31 μmol, 50.08% yield) as a yellow solid. LC/MS(ESI⁺) [(M+H)⁺]: 584.8.

### Step 12:

tert-butyl N-[(1R,4R,7R)-2-[2-(11-cyclopropyl-1,9-diazatricyclo[6.3.1.04,12]dodeca-2,4(12), 5,7-tetraen-2-yl)-7-fluoro-1-methyl-benzimidazole-5-carbonyl]-2-azabicyclo[2.2.1]heptan-7-yl]carbamate (30.0 mg, 51.31 μmol) was dissolved in HCl (4M)/Dioxane=1/2 (3 mL). The resulting mixture was stirred at RT for 30 min. After removal of the solvent in vacuo, the residue was purified by pre-HPLC to afford [(1R,4R,7R)-7-amino-2-azabicyclo[2.2.1]heptan-2-yl]-[2-(11-cyclopropyl-1,9-diazatricyclo[6.3.1.04,12]dodeca-2,4(12), 5,7-tetraen-2-yl)-7-fluoro-1-methyl-benzimidazol-5-yl]methanone (10.0 mg, 20.64 μmol, 40.22% yield) as a yellow solid. LC/MS(ESI⁺) [(M+H)⁺]: 484.7.

### Example 109 and Example 110

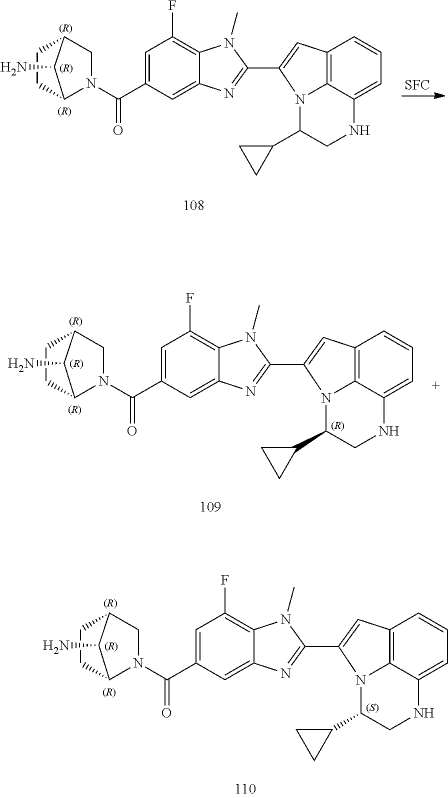

Example 108 was chirally separated by SFC to give Example 109 and Example 110 (Column: AD-H 4.6*100 mm, 5 um; Co-solvent: MeOH/ACN=1/1[0.2% NH3(7 M in MeOH)]; Flow-rate: 3 mL/min; first peak: Example 110; second peak: Example 109). Example 109: ¹H NMR (400 MHz, DMSO-d₆) δ 7.56 (d, J=1.1 Hz, 1H), 7.22 (d, J=11.8 Hz, 1H), 6.99 (s, 1H), 6.93 (d, J=7.9 Hz, 1H), 6.88 (t, J=7.6 Hz, 1H), 6.39 (d, J=7.0 Hz, 1H), 6.21 (d, J=3.0 Hz, 1H), 4.59 (d, J=9.3 Hz, 1H), 4.14 (d, J=2.9 Hz, 3H), 3.73-3.48 (m, 4H), 3.17 (s, 1H), 3.07-2.97 (m, 1H), 2.20 (d, J=3.8 Hz, 1H), 1.94 (d, J=11.7 Hz, 2H), 1.74 (d, J=10.2 Hz, 1H), 1.46-1.36 (m, 1H), 0.99 (qd, J=8.6, 4.1 Hz, 1H), 0.33 (tt, J=8.8, 4.7 Hz, 1H), 0.19 (dq, J=10.1, 5.1 Hz, 1H), 0.07 (t, J=4.6 Hz, 1H), −0.69 (dd, J=9.0, 4.3 Hz, 1H). Example 110: ¹H NMR (400 MHz, DMSO-d₆) δ 7.57 (d, J=1.2 Hz, 1H), 7.22 (dd, J=12.0, 1.2 Hz, 1H), 6.98 (d, J=1.4 Hz, 1H), 6.89 (dd, J=15.7, 8.4 Hz, 2H), 6.39 (d, J=7.1 Hz, 1H), 6.21 (d, J=2.9 Hz, 1H), 4.58 (d, J=9.4 Hz, 1H), 4.14 (d, J=2.7 Hz, 3H), 3.74-3.48 (m, 4H), 3.18 (s, 1H), 3.03-3.01 (m, 1H), 2.20 (d, J=4.0 Hz, 1H), 2.01-1.88 (m, 2H), 1.74 (d, J=6.7 Hz, 1H), 1.48-1.33 (m, 1H), 0.99 (qt, J=8.7, 4.8 Hz, 1H), 0.33 (tt, J=8.8, 4.7 Hz, 1H), 0.20 (dq, J=9.8, 5.0 Hz, 1H), 0.07 (q, J=5.3 Hz, 1H), −0.66 (dq, J=9.7, 4.9 Hz, 1H).

### Example 111, Example 112 and Example 113

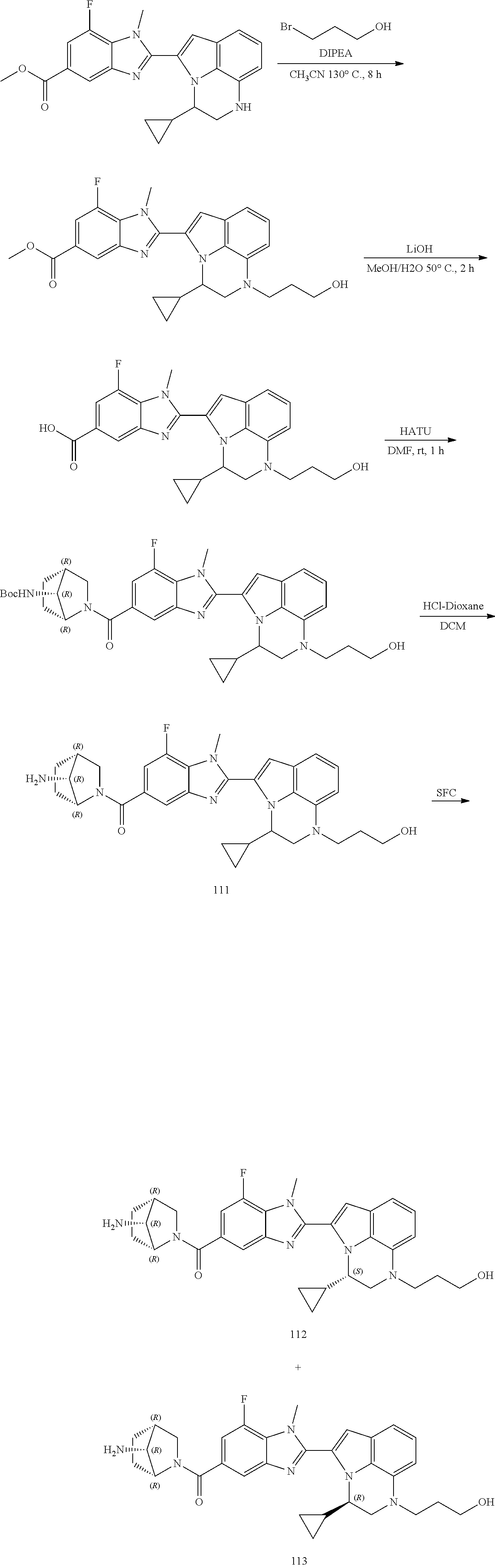

### Step 1:

A mixture of 3-bromopropan-1-ol (343.6 mg, 2.47 mmol) and methyl 2-(11-cyclopropyl-1,9-diazatricyclo[6.3.1.04,12]dodeca-2,4(12), 5,7-tetraen-2-yl)-7-fluoro-1-methyl-benzimidazole-5-carboxylate (200.0 mg, 494.52 μmol, intermediate of example 108) in acetonitrile (4 mL) was stirred at 130° C. for 8 h. Upon completion, the mixture was concentrated in vacuo. The crude material was purified by flash column chromatography on silica gel (eluting with 0-100% EA in PE) to give methyl 2-[11-cyclopropyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carboxylate (150.0 mg, 324.31 μmol, 65.5% yield) as a yellow solid. LC/MS (ESI⁺): m/z 462.8 [(M+H)⁺].

### Step 2:

A mixture of methyl 2-[11-cyclopropyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carboxylate (150.0 mg, 324.31 μmol) and LiOH (27.2 mg, 648.63 μmol) in H₂O/MeOH mixed solvents (3 mL, 1:1) was stirred at 50° C. for 2 h. Upon completion, the mixture was acidified with 3 M hydrochloric acid. The mixture was diluted with EtOAc (50 mL) and washed with water (25 mL). The organic layer was dried over sodium sulfate, filtered, and concentrated in vacuo. The crude material was purified by flash column chromatography on silica gel (eluting with 0-10% EA in PE) to give 2-[11-cyclopropyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carboxylic acid (90.0 mg, 200.67 μmol, 61.8% yield) as a yellow liquid. LC/MS (ESI⁺): m/z 448.8 [(M+H)⁺].

### Step 3:

A mixture of 2-[11-cyclopropyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carboxylic acid (90.0 mg, 200.67 μmol), tert-butyl N-[(1R,4R,7R)-2-azabicyclo[2.2.1]heptan-7-yl]carbamate (51.1 mg, 240.81 μmol), HATU (114.4 mg, 301.01 μmol) and DIPEA (51.9 mg, 401.35 μmol) in DMF (4 mL) was stirred at RT for 1 h. Upon completion, the mixture was diluted with EtOAc (50 mL) and washed with water (25 mL). The organic layer was dried over sodium sulfate, filtered, and concentrated in vacuo. The crude material was purified by flash column chromatography on silica gel (eluting with 0-100% EA in PE) to give tert-butyl N-[(1R,4R,7R)-2-[2-[11-cyclopropyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carbonyl]-2-azabicyclo[2.2.1]heptan-7-yl]carbamate (100.0 mg, 155.58 μmol, 77.5% yield) as a yellow solid. LC/MS (ESI⁺): m/z 642.8 [(M+H)⁺].

### Step 4:

To a solution of tert-butyl N-[(1R,4R,7R)-2-[2-[11-cyclopropyl-9-(3-hydroxypropyl)-1,9-diazatricyclo[6.3.1.04,12]dodeca-2,4(12), 5,7-tetraen-2-yl]-7-fluoro-1-methyl-benzimidazole-5-carbonyl]-2-azabicyclo[2.2.1]heptan-7-yl]carbamate (100.0 mg, 155.58 μmol) in DCM (1.5 mL) was added a solution of HCl in dioxane (4 M, 3 mL). The resulting mixture was stirred at RT for 30 min. Upon completion, solvent was removed in vacuo and the residue was purified by prep-HPLC to afford [(1R,4R,7R)-7-amino-2-azabicyclo[2.2.1]heptan-2-yl]-[2-[11-cyclopropyl-9-(3-hydroxypropyl)-1,9-diazatricyclo[6.3.1.04,12]dodeca-2,4(12), 5,7-tetraen-2-yl]-7-fluoro-1-methyl-benzimidazol-5-yl]methanone (60.0 mg, 110.57 μmol, 71.0% yield) as a yellow solid. LC/MS (ESI⁺): m/z 542.8 [(M+H)⁺].

### Step 5:

Example 111 was further separated by SFC to afford Example 112 (27.0 mg) and Example 113 (29.0 mg) (AD-H 4.6*100 mm, 5 um; MeOH/ACN=1/1[0.2% NH3(7 M in MeOH)]; Flow-rate: 3.0 mL/min; firstpeak: Example 112; second peak: Example 113). Example 112: LC/MS (ESI⁺): m/z 542.8 [(M+H)⁺]; ¹H NMR (400 MHz, DMSO-d₆) δ 7.56 (d, J=1.2 Hz, 1H), 7.27-7.18 (m, 1H), 7.01 (s, 1H), 6.99-6.91 (m, 2H), 6.50-6.41 (m, 1H), 4.64 (d, J=9.3 Hz, 1H), 4.54 (t, J=5.0 Hz, 1H), 4.14 (d, J=2.3 Hz, 3H), 3.74-3.63 (m, 2H), 3.58-3.45 (m, 5H), 3.19 (s, 1H), 3.09-3.02 (m, 1H), 2.21 (s, 2H), 1.95 (s, 2H), 1.86-1.71 (m, 3H), 1.47-1.38 (m, 1H), 1.24 (s, 1H), 0.95 (ddt, J=13.3, 8.8, 4.4 Hz, 1H), 0.33 (td, J=8.9, 4.4 Hz, 1H), 0.21 (dd, J=9.8, 5.0 Hz, 1H), 0.06 (s, 1H), −0.62-−0.70 (m, 1H). Example 113: LC/MS (ESI⁺): m/z 542.8 [(M+H)⁺]; H NMR (400 MHz, DMSO-d₆) δ 7.56 (d, J=1.2 Hz, 1H), 7.27-7.18 (m, 1H), 7.01 (s, 1H), 6.99-6.91 (m, 2H), 6.50-6.41 (m, 1H), 4.64 (d, J=9.3 Hz, 1H), 4.54 (t, J=5.0 Hz, 1H), 4.14 (d, J=2.3 Hz, 3H), 3.74-3.63 (m, 2H), 3.58-3.45 (m, 5H), 3.19 (s, 1H), 3.09-3.02 (m, 1H), 2.21 (s, 2H), 1.95 (s, 2H), 1.86-1.71 (m, 3H), 1.47-1.38 (m, 1H), 1.24 (s, 1H), 0.95 (ddt, J=13.3, 8.8, 4.4 Hz, 1H), 0.33 (td, J=8.9, 4.4 Hz, 1H), 0.21 (dd, J=9.8, 5.0 Hz, 1H), 0.06 (s, 1H), −0.62-−0.70 (m, 1H).

### Example 114 Synthesis of 5-(5-((1R,4R,7R)-7-amino-2-azabicyclo[2.2.1]heptane-2-carbonyl)-7-fluoro-1-methyl-1H-benzo[d]imidazol-2-yl)-3-cyclopropyl-2,3-dihydro-1H-pyrrolo[1,2,3-de]quinoxaline-9-carbonitrile

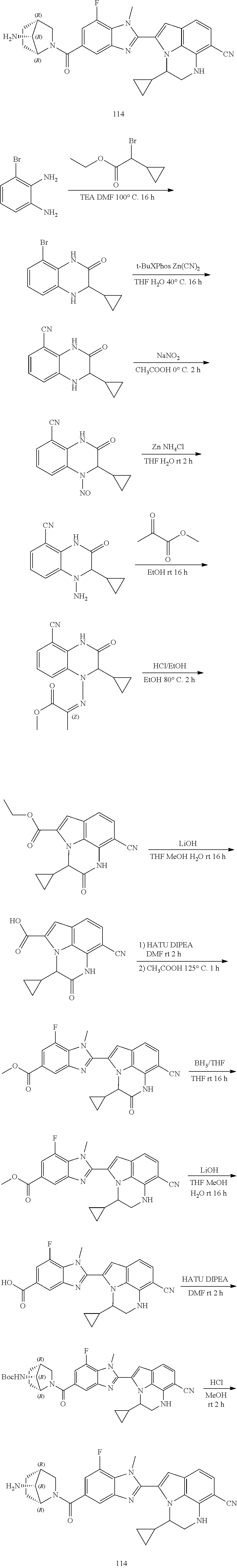

Step 1: A mixture of methyl 2-bromo-2-cyclopropyl-acetate (4.6 g, 24 mmol), 3-bromobenzene-1,2-diamine (6.77 g, 36.22 mmol), and TEA (7.33 g, 72.44 mmol) in DMF (50 mL) was stirred at 100° C. for 16 h. The mixture was cooled to RT and concentrated in vacuo. The residue was diluted with water (100 mL) and extracted with EtOAc (100 mL×2). The combined organic layers were dried over Na₂SO₄, filtered, and concentrated in vacuo. The residue was purified by flash chromatography on silica gel (eluting with 0-30% EA in PE) to give 8-bromo-3-cyclopropyl-3,4-dihydroquinoxalin-2(1H)-one (3.2 g, 11.98 mmol, 49.6% yield) as a yellow solid. LC/MS (ESI⁺): m/z 266.7 [(M+H)⁺].

### Step 2:

A mixture of 8-bromo-3-cyclopropyl-3,4-dihydro-1H-quinoxalin-2-one (3.2 g, 11.98 mmol), zinc cyanide (2.81 g, 23.96 mmol) and tBuXPhos-Pd-G3 (285.49 mg, 359.39 μmol) in THF/H₂O mixed solvents (60 mL, 1:1) was heated at 40° C. for 16 h. Then the reaction was cooled to RT, diluted with EA, washed with brine. The organic phase was dried over Na₂SO₄, filtered, and concentrated in vacuo. The crude mixture was purified by column chromatography on silica gel (eluting with 0-7% EA in PE) to give 2-cyclopropyl-3-oxo-1,2,3,4-tetrahydroquinoxaline-5-carbonitrile (2.5 g, 11.72 mmol, 97.8% yield) as a yellow solid. LC/MS (ESI⁺): m/z 213.8 [(M+H)⁺].

### Step 3:

To a stirred solution of 2-cyclopropyl-3-oxo-2,4-dihydro-1H-quinoxaline-5-carbonitrile (2.5 g, 11.72 mmol) in CH₃COOH (30 mL) was added a solution of sodium nitrite (808.97 mg, 11.72 mmol) in H₂O (10 mL) at 0° C., and the resulting mixture was stirred at 0° C. for 2 h. Upon completion, the mixture was diluted with DCM, washed with brine. The organic phase was dried over Na₂SO₄, filtered, and concentrated in vacuo to give 2-cyclopropyl-1-nitroso-3-oxo-1,2,3,4-tetrahydroquinoxaline-5-carbonitrile (2.8 g, 11.56 mmol, 98.5% yield) as a yellow solid, LC/MS (ESI⁺): m/z 212.9 [(M+H-30)⁺].

### Step 4:

To a stirred solution of 2-cyclopropyl-1-nitroso-3-oxo-2,4-dihydroquinoxaline-5-carbonitrile (2.8 g, 11.56 mmol) in THF (30 mL) was added a solution of ammonium chloride (2.47 g, 46.24 mmol) in H₂O (30 mL) followed by zinc powder (3.02 g, 46.24 mmol). The resulting mixture was stirred at RT for 2 h. Upon completion, the mixture was filtered and the filtrate was extracted with EtOAc (30 mL×2). The combined organic layers were dried over Na₂SO₄, filtered, and concentrated in vacuo to afford 1-amino-2-cyclopropyl-3-oxo-1,2,3,4-tetrahydroquinoxaline-5-carbonitrile (2.5 g) as a yellow solid which was used in the next step directly without further purification. LC/MS (ESI⁺): m/z 228.8 [(M+H)⁺].

### Step 5:

A mixture of 1-amino-2-cyclopropyl-3-oxo-2,4-dihydroquinoxaline-5-carbonitrile (2.5 g, 10.95 mmol) and methyl 2-oxopropanoate (1.12 g, 10.95 mmol) in EtOH (30 mL) was stirred at RT for 16 h and then concentrated in vacuo. The residue was slurried in DCM (12.5 mL, 5 v/w) and PE (62.5 mL, 25 v/w), filtered and the filter cake was dried in vacuo to afford methyl (Z)-2-((5-cyano-2-cyclopropyl-3-oxo-3,4-dihydroquinoxalin-1(2H)-yl)imino)propanoate (3.0 g, 9.61 mmol, 87.7% yield) as a yellow solid. LC/MS (ESI⁺): m/z 312.8 [(M+H)⁺].

### Step 6:

To a stirred solution of methyl (2Z)-2-[(5-cyano-2-cyclopropyl-3-oxo-2,4-dihydroquinoxalin-1-yl)imino]propanoate (3.0 g, 9.61 mmol) in EtOH (30 mL) was added a solution of HCl in EtOH (4 M, 30 mL). The resulting mixture was heated to 80° C. and stirred for 2 h. After cooling to RT, the mixture was concentrated in vacuo. The residue was diluted with DCM and washed with water. The organic phase was dried over Na₂SO₄, filtered, and concentrated in vacuo. The crude mixture was purified by column chromatography on silica gel (eluting with 0-20% EA in PE) to afford ethyl 9-cyano-3-cyclopropyl-2-oxo-2,3-dihydro-1H-pyrrolo[1,2,3-de]quinoxaline-5-carboxylate (1.32 g, 4.27 mmol, 44.4% yield) as a yellow solid. LC/MS (ESI⁺): m/z 309.8[(M+H)⁺].

### Step 7:

To a stirred solution of ethyl 7-cyano-11-cyclopropyl-10-oxo-1,9-diazatricyclo[6.3.1.0^{4,12}]dodeca-2,4,6,8(12)-tetraene-2-carboxylate (1.32 g, 4.27 mmol) in THF/MeOH mixed solvetns (30 mL, 2:1) was added LiOH aqueous solution (1.0 M, 12.8 mL). The mixture was stirred at RT for 16 h, and then acidified to pH=5-6 with 3 M hydrochloric acid aqueous solution. The mixture was extracted with EA (20 mL×3) and the organic phase was dried over anhydrous sodium sulfate, filtered and concentrated in vacuo to give 7-cyano-11-cyclopropyl-10-oxo-1,9-diazatricyclo[6.3.1.0^{4,12}]dodeca-2,4,6,8(12)-tetraene-2-carboxylic acid (1.2 g, 4.27 mmol, 99.9% yield) as a white solid. LC/MS (ESI⁺): m/z 281.8 [(M+H)⁺].

### Step 8:

A mixture of 7-cyano-11-cyclopropyl-10-oxo-1,9-diazatricyclo[6.3.1.0^{4,12}]dodeca-2,4,6,8(12)-tetraene-2-carboxylic acid (1.2 g, 4.27 mmol), DIPEA (1.65 g, 12.80 mmol), HATU (1.95 g, 5.12 mmol), and methyl 3-amino-5-fluoro-4-(methylamino)benzoate (845.6 mg, 4.27 mmol, synthesized according to WO2021222353) in DMF (20 mL) was stirred at RT for 2 h. Upon completion, the mixture was diluted with EA and washed with brine. The organic phase was dried over anhydrous sodium sulfate, filtered, and concentrated in vacuo. The residue was then stirred in CH₃COOH (20 mL) at 120° C. for 1 h. After cooling to RT, the mixture was concentrated in vacuo. The residue was diluted with EA (80 mL) and washed with saturated Na₂CO₃ solution. The organic phase was dried over Na₂SO₄, filtered and concentrated in vacuo. The residue was purified by flash column chromatography on silica gel (eluting with 0-7% MeOH in DCM) to give methyl 2-(9-cyano-3-cyclopropyl-2-oxo-2,3-dihydro-1H-pyrrolo[1,2,3-de]quinoxalin-5-yl)-7-fluoro-1-methyl-1H-benzo[d]imidazole-5-carboxylate (820 mg, 1.85 mmol, 43.3% yield) as a yellow solid. LC/MS (ESI⁺): m/z 443.8 [(M+H)⁺].

### Step 9:

To a stirred solution of methyl 2-(7-cyano-11-cyclopropyl-10-oxo-1,9-diazatricyclo[6.3.1.0^{4,12}]dodeca-2,4,6,8(12)-tetraen-2-yl)-7-fluoro-1-methyl-benzimidazole-5-carboxylate (220 mg, 496.13 μmol) in anhydrous THF (3 mL) was slowly added borane tetrahydrofuran (1 M, 1.5 mL) at 0° C. After being stirred at RT for 16 h, the mixture was quenched with MeOH at 0° C. and concentrated in vacuo. The residue was diluted with 2 M HCl aqueous solution (3 mL) and the mixture was stirred at RT for 1 h before 4 M NaOH aqueous solution was added to basify the mixture to pH ˜8. The mixture was extracted with DCM (30 mL×3), and the combined organic layers were dried over anhydrous sodium sulfate, filtered, and concentrated in vacuo. The residue was purified by flash column chromatography on silica gel (eluting with 0-4% MeOH in DCM) to give methyl 2-(7-cyano-11-cyclopropyl-1,9-diazatricyclo[6.3.1.0^{4,12}]dodeca-2,4,6,8(12)-tetraen-2-yl)-7-fluoro-1-methyl-benzimidazole-5-carboxylate (130 mg, 302.72 μmol, 61.0% yield) as a yellow solid. LC/MS (ESI⁺): m/z 429.7 [(M+H)⁺].

### Step 10:

To a stirred solution of methyl 2-(7-cyano-11-cyclopropyl-1,9-diazatricyclo[6.3.1.0^{4,12}]dodeca-2,4,6,8(12)-tetraen-2-yl)-7-fluoro-1-methyl-benzimidazole-5-carboxylate (130 mg, 302.72 μmol) in THF/MeOH mixed solvents (3 mL, 2:1) was added LiOH aqueous solution (1.0 M, 0.90 mL). The mixture was stirred at RT for 2 h, before 3 M hydrochloric acid aqueous solution was added to acidify the mixture to pH=5-6. The mixture was then extracted with DCM (20 mL×3). The combined organic layers were dried over anhydrous sodium sulfate, filtered, and concentrated in vacuo to give 2-(7-cyano-11-cyclopropyl-1,9-diazatricyclo[6.3.1.0^{4,12}]dodeca-2,4,6,8(12)-tetraen-2-yl)-7-fluoro-1-methyl-benzimidazole-5-carboxylic acid (120 mg, 288.86 μmol, 95.4% yield) as a white solid. LC/MS (ESI⁺): m/z 415.8 [(M+H)⁺].

### Step 11:

A mixture of 2-(7-cyano-11-cyclopropyl-1,9-diazatricyclo[6.3.1.0^{4,12}]dodeca-2,4,6,8(12)-tetraen-2-yl)-7-fluoro-1-methyl-benzimidazole-5-carboxylic acid (120 mg, 288.86 μmol), DIPEA (44.8 mg, 346.64 μmol), HATU (329.5 mg, 866.59 μmol), and tert-butyl ((1R,4R,7R)-2-azabicyclo[2.2.1]heptan-7-yl)carbamate (61.3 mg, 288.86 μmol) in DMF (2 mL) was stirred at RT for 2 h, before being diluted with EA and washed with brine. The organic phase was dried over anhydrous sodium sulfate, filtered, and concentrated in vacuo. The residue was purified by flash column chromatography on silica gel (eluting with 0-8% MeOH in DCM) to give tert-butyl ((1R,4R,7R)-2-(2-(9-cyano-3-cyclopropyl-2,3-dihydro-1H-pyrrolo[1,2,3-de]quinoxalin-5-yl)-7-fluoro-1-methyl-1H-benzo[d]imidazole-5-carbonyl)-2-azabicyclo[2.2.1]heptan-7-yl)carbamate (70 mg, 114.81 μmol, 39.7% yield) as a white solid. LC/MS (ESI⁺): m/z 609.7 [(M+H)⁺].

### Step 12:

To a solution of tert-butyl N-[(1R,4R,7R)-2-[2-(7-cyano-11-cyclopropyl-1,9-diazatricyclo[6.3.1.0^{4,12}]dodeca-2,4,6,8(12)-tetraen-2-yl)-7-fluoro-1-methyl-benzimidazole-5-carbonyl]-2-azabicyclo[2.2.1]heptan-7-yl]carbamate (70 mg, 114.81 μmol) in MeOH (2 mL) was added 4 M HCl in dioxane (2 mL) and the resulting mixture was stirred at RT for 2 h. Upon completion, the reaction mixture was basified to pH=8 with saturated Na₂CO₃ solution and then extracted with DCM (30 mL×3). The combined organic layers were dried over anhydrous sodium sulfate, filtered, and concentrated in vacuo. The residue was purified by flash column chromatography on silica gel (eluting with 0-10% MeOH in DCM) to give 2-[5-[(1R,4R,7R)-7-amino-2-azabicyclo[2.2.1]heptane-2-carbonyl]-7-fluoro-1-methyl-benzimidazol-2-yl]-11-cyclopropyl-1,9-diazatricyclo[6.3.1.0^{4,12}]dodeca-2,4,6,8(12)-tetraene-7-carbonitrile (36 mg, 70.65 μmol, 61.5% yield) as a white solid. LC/MS (ESI⁺): m/z 509.8 [(M+H)⁺]. ¹H NMR (400 MHz, DMSO-d₆) δ 7.59 (d, J=5.9 Hz, 1H), 7.35-7.27 (m, 1H), 7.27-7.21 (m, 1H), 7.12 (s, 1H), 7.06 (d, J=8.4 Hz, 1H), 6.99 (d, J=8.4 Hz, 1H), 4.66-4.58 (m, 1H), 4.13 (t, J=2.5 Hz, 3H), 3.78 (dd, J=12.4, 3.2 Hz, 1H), 3.71 (dd, J=6.2, 3.2 Hz, 1H), 3.68-3.58 (m, 1H), 3.55-3.46 (m, 1H), 3.18 (s, 1H), 3.11-2.97 (m, 2H), 2.20 (d, J=3.9 Hz, 1H), 2.03-1.81 (m, 3H), 1.80-1.61 (m, 2H), 1.49-1.20 (m, 2H), 0.90 (dtt, J=13.9, 9.8, 4.9 Hz, 1H), 0.34 (tt, J=8.8, 4.7 Hz, 1H), 0.24 (dp, J=10.0, 5.1 Hz, 1H), 0.08 (tt, J=6.5, 3.4 Hz, 1H), −0.71 (dh, J=9.8, 4.8 Hz, 1H).

### Example 115 and Example 116

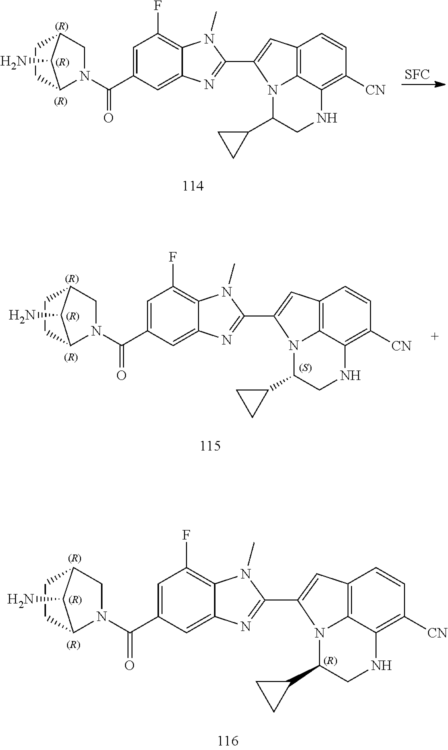

Example 114 was separated by SFC to give Example 115 and Example 116 (AD-H 4.6*100 mm, 5 um; EtOH[1% NH₃(7 M in MeOH)]; Flow-rate: 3.0 mL/min; first peak: Example 115; second peak: Example 116). Example 115: ¹H NMR (400 MHz, DMSO-d₆) δ 7.37 (s, 1H), 7.15-6.99 (m, 2H), 6.90 (s, 1H), 6.84 (d, J=8.4 Hz, 1H), 6.77 (d, J=8.4 Hz, 1H), 4.40 (d, J=9.3 Hz, 1H), 3.91 (d, J=2.3 Hz, 3H), 3.55 (dd, J=8.7, 3.8 Hz, 2H), 3.50-3.42 (m, 1H), 3.34-3.26 (m, 1H), 3.04 (s, 3H), 2.85 (dd, J=12.7, 10.0 Hz, 2H), 2.07 (d, J=13.4 Hz, 1H), 1.78-1.62 (m, 3H), 1.61-1.47 (m, 1H), 1.25 (t, J=9.1 Hz, 1H), 0.67 (ddq, J=13.6, 9.8, 4.4 Hz, 1H), 0.12 (tt, J=8.8, 4.8 Hz, 1H), −0.14 (tt, J=9.3, 4.4 Hz, 1H), −0.94 (dq, J=9.7, 4.9 Hz, 1H). Example 116: ¹H NMR (400 MHz, DMSO-d₆) δ 7.46-7.33 (m, 1H), 7.11-6.97 (m, 2H), 6.88 (s, 1H), 6.82 (d, J=8.5 Hz, 1H), 6.75 (d, J=8.4 Hz, 1H), 4.41-4.34 (m, 1H), 3.89 (d, J=2.5 Hz, 3H), 3.54 (dd, J=12.3, 3.3 Hz, 1H), 3.44 (d, J=12.9 Hz, 2H), 3.26 (dt, J=11.1, 3.0 Hz, 1H), 2.94 (s, 1H), 2.80 (dd, J=14.3, 10.0 Hz, 1H), 1.95 (d, J=3.9 Hz, 1H), 1.88 (dd, J=7.5, 5.1 Hz, 1H), 1.79-1.71 (m, 1H), 1.69 (s, 1H), 1.44(h, J=10.1 Hz, 2H), 1.22-1.08 (m, 1H), 1.01 (d, J=9.4 Hz, 1H), 0.93-0.74 (m, 1H), 0.65 (dtt, J=16.0, 11.4, 5.3 Hz, 2H), 0.11 (tt, J=8.8, 4.8 Hz, 1H), −0.15 (tt, J=9.3, 4.5 Hz, 1H), −0.93 (dq, J=9.6, 4.9 Hz, 1H).

### Example 117 Preparation of (R)-5-(5-((1R,4R,7R)-7-amino-2-azabicyclo[2.2.1]heptane-2-carbonyl)-7-fluoro-1-methyl-1H-benzo[d]imidazol-2-yl)-3-cyclopropyl-2,3-dihydro-1H-pyrrolo[1,2,3-de]quinoxaline-8-carbonitrile

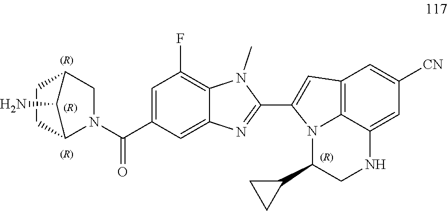

Prepared in analogous manner as for Example 114 followed by SFC seperation (Column: AS-3 4.6*100 mm 3 um; Co-solvent: EtOH [with 1% NH₃ (7 M in MeOH)]; Flowrate: 3.0 mL/min; Column Temperature: 40° C.). LC/MS (ESI⁺): m/z 509.7 [(M+H)⁺]. ¹H NMR (400 MHz, DMSO-d₆) δ 7.71-7.57 (m, 1H), 7.47 (d, J=1.2 Hz, 1H), 7.35-7.22 (m, 1H), 7.15 (s, 1H), 6.73 (d, J=2.9 Hz, 1H), 6.61 (d, J=1.3 Hz, 1H), 4.62 (d, J=9.4 Hz, 1H), 4.13 (d, J=2.7 Hz, 3H), 3.75-3.58 (m, 3H), 3.50 (dt, J=11.1, 3.0 Hz, 1H), 3.20-2.97 (m, 2H), 2.23-1.66 (m, 5H), 1.30-1.18 (m, 2H), 0.95 (qt, J=8.7, 5.1 Hz, 1H), 0.34 (tt, J=8.9, 4.9 Hz, 1H), 0.22 (dp, J=10.0, 5.1 Hz, 1H), 0.09 (dp, J=9.4, 4.5 Hz, 1H), −0.69 (dq, J=9.8, 5.0 Hz, 1H).

### Example 118 Preparation of (S)-5-(5-((1R,4R,7R)-7-amino-2-azabicyclo[2.2.1]heptane-2-carbonyl)-7-fluoro-1-methyl-1H-benzo[d]imidazol-2-yl)-3-cyclopropyl-2,3-dihydro-1H-pyrrolo[1,2,3-de]quinoxaline-8-carbonitrile

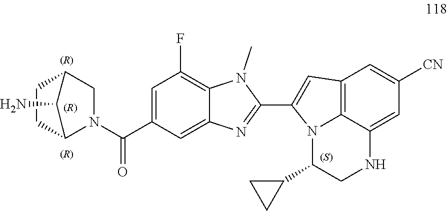

Prepared in analogous manner as for Example 114 followed by SFC seperation (Column: AS-3 4.6*100 mm 3 um; Co-solvent: EtOH [with 1% NH₃ (7 M in MeOH)]; Flowrate: 3.0 mL/min; Column Temperature: 40° C.). LC/MS (ESI⁺): m/z 509.7 [(M+H)⁺]. ¹H NMR (400 MHz, DMSO-d₆) δ 7.72-7.56 (m, 1H), 7.46 (d, J=6.7 Hz, 1H), 7.36-7.20 (m, 1H), 7.14 (d, J=9.8 Hz, 1H), 6.75 (t, J=4.0 Hz, 1H), 6.60 (d, J=3.3 Hz, 1H), 4.63 (d, J=9.5 Hz, 1H), 4.13 (d, J=3.8 Hz, 3H), 3.65 (q, J=11.8 Hz, 3H), 3.55-3.46 (m, 1H), 3.20-2.95 (m, 2H), 2.23-1.68 (m, 5H), 1.30-1.18 (m, 2H), 0.95 (pd, J=7.7, 3.7 Hz, 1H), 0.35 (tt, J=9.2, 4.8 Hz, 1H), 0.22 (dq, J=9.8, 4.9 Hz, 1H), 0.08 (s, 1H), −0.70 (dq, J=10.2, 5.1 Hz, 1H).

### Example 119 Preparation of (R)-5-(5-((1R,4R,7R)-7-amino-2-azabicyclo[2.2.1]heptane-2-carbonyl)-7-fluoro-1-methyl-1H-benzo[d]imidazol-2-yl)-3-cyclopropyl-2,3-dihydro-1H-pyrrolo[1,2,3-de]quinoxaline-7-carbonitrile

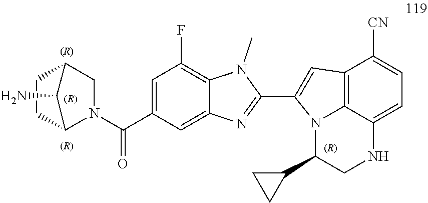

Prepared in analogous manner as for Example 114 followed by SFC seperation (Column: AS-3 4.6*100 mm 3 um; Co-solvent: EtOH [with 1% NH₃ (7 M in MeOH)]; Flowrate: 3.0 mL/min; Column Temperature: 40° C.). LC/MS (ESI⁺): m/z 509.7 [(M+H)⁺]. ¹H NMR (400 MHz, DMSO-d₆) δ 7.72-7.56 (m, 1H), 7.41 (dd, J=7.9, 1.2 Hz, 1H), 7.35-7.20 (m, 2H), 7.11 (d, J=1.2 Hz, 1H), 6.47 (dd, J=7.9, 1.2 Hz, 1H), 4.62 (d, J=9.6 Hz, 1H), 4.16 (d, J=3.0 Hz, 3H), 3.80-3.45 (m, 4H), 3.20-2.96 (m, 2H), 2.22-2.07 (m, 1H), 2.03-1.61 (m, 4H), 1.30-1.20 (m, 2H), 0.88 (ddt, J=16.9, 13.0, 6.0 Hz, 1H), 0.33 (dq, J=9.2, 4.6 Hz, 1H), 0.21 (dt, J=10.1, 5.0 Hz, 1H), 0.08 (dd, J=9.3, 5.0 Hz, 1H), −0.74 (dd, J=9.6, 5.1 Hz, 1H).

### Example 120 Preparation of(S)-5-(5-((1R,4R,7R)-7-amino-2-azabicyclo[2.2.1]heptane-2-carbonyl)-7-fluoro-1-methyl-1H-benzo[d]imidazol-2-yl)-3-cyclopropyl-2,3-dihydro-1H-pyrrolo[1,2,3-de]quinoxaline-7-carbonitrile

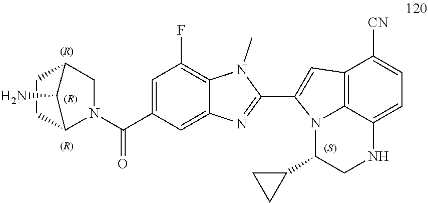

Prepared in analogous manner as for Example 114 followed by SFC seperation (Column: AS-3 4.6*100 mm 3 um; Co-solvent: EtOH [with 1% NH₃ (7 M in MeOH)]; Flowrate: 3.0 mL/min; Column Temperature: 40° C.). LC/MS (ESI⁺): m/z 509.7 [(M+H)⁺]. ¹H NMR (400 MHz, DMSO-d₆) δ 7.71-7.57 (m, 1H), 7.41 (d, J=7.9 Hz, 1H), 7.36-7.21 (m, 2H), 7.11 (d, J=1.4 Hz, 1H), 6.47 (d, J=7.9 Hz, 1H), 4.61 (d, J=9.3 Hz, 1H), 4.16 (d, J=2.7 Hz, 3H), 3.84-3.59 (m, 3H), 3.50 (dt, J=10.9, 3.0 Hz, 1H), 3.20-2.99 (m, 2H), 2.23-2.07 (m, 1H), 2.03-1.61 (m, 4H), 1.30-1.20 (m, 2H), 0.88 (dtd, J=17.7, 8.2, 4.8 Hz, 1H), 0.34 (tt, J=8.9, 4.8 Hz, 1H), 0.22 (dp, J=10.2, 5.2 Hz, 1H), 0.08 (dt, J=9.1, 4.7 Hz, 1H), −0.71 (dq, J=9.8, 5.1 Hz, 1H).

### Example 121 Preparation of [(1R,4R,7R)-7-amino-2-azabicyclo[2.2.1]heptan-2-yl]-[2-[11-cyclopropyl-7-(trifluoromethyl)-1,9-diazatricyclo[6.3.1.0^{4,12}]dodeca-2,4,6,8(12)-tetraen-2-yl]-7-fluoro-1-methyl-benzimidazol-5-yl]methanone

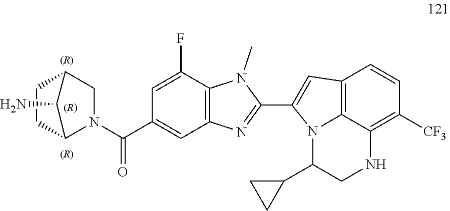

Prepared in analogous manner as for Example 114. LC/MS (ESI⁺): m/z 552.7 [(M+H)⁺]. ¹H NMR (400 MHz, DMSO-d₆) δ 7.71-7.56 (m, 1H), 7.35-7.20 (m, 1H), 7.14-6.98 (m, 3H), 6.60 (d, J=3.5 Hz, 1H), 4.64-4.53 (m, 1H), 4.13 (t, J=2.6 Hz, 3H), 3.81-3.57 (m, 3H), 3.51 (dt, J=11.1, 2.9 Hz, 2H), 3.18 (s, 1H), 3.10-2.96 (m, 1H), 2.24-2.09 (m, 1H), 2.04-1.80 (m, 3H), 1.78-1.59 (m, 1H), 1.49-1.30 (m, 1H), 0.93 (ddq, J=13.5, 9.0, 5.0 Hz, 1H), 0.35 (tt, J=8.8, 4.7 Hz, 1H), 0.23 (dp, J=10.1, 5.1 Hz, 1H), 0.09 (q, J=7.2 Hz, 1H), −0.73 (dq, J=10.1, 5.0 Hz, 1H).

### Example 122 Synthesis of ((1R,4R,7R)-7-amino-2-azabicyclo[2.2.1]heptan-2-yl)(2-(3-cyclopropyl-8-fluoro-2,3-dihydro-1H-pyrrolo[1,2,3-de]quinoxalin-5-yl)-7-fluoro-1-methyl-1H-benzo[d]imidazol-5-yl)methanone

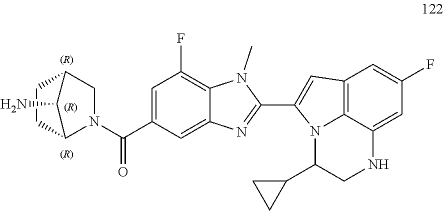

Prepared in analogous manner as for Example 114. LC/MS (ESI⁺): m/z 502.7 [(M+H)⁺]. ¹H NMR (400 MHz, DMSO-d₆) δ 7.56 (dd, J=5.7, 1.2 Hz, 1H), 7.22 (dd, J=12.0, 1.2 Hz, 1H), 6.97 (s, 1H), 6.64 (dd, J=10.2, 2.1 Hz, 1H), 6.55 (d, J=3.1 Hz, 1H), 6.22 (dd, J=11.2, 2.1 Hz, 1H), 4.56 (d, J=9.3 Hz, 1H), 4.13 (t, J=2.4 Hz, 3H), 3.74-3.54 (m, 3H), 3.50 (q, J=4.2 Hz, 1H), 3.18 (s, 1H), 3.09-2.97 (m, 2H), 2.20 (s, 1H), 2.05-1.87 (m, 3H), 1.77-1.63 (m, 1H), 1.49-1.31 (m, 2H), 1.24 (d, J=5.7 Hz, 1H), 1.01-0.81 (m, 2H), 0.33 (tt, J=8.8, 4.7 Hz, 1H), 0.19 (dp, J=9.8, 4.8 Hz, 1H), 0.07 (q, J=4.7 Hz, 1H), −0.69 (dh, J=9.9, 4.9 Hz, 1H).

### Example 123 Synthesis of ((1R,4R,7R)-7-amino-2-azabicyclo[2.2.1]heptan-2-yl)(2-(3-cyclopropyl-9-(methylsulfonyl)-2,3-dihydro-1H-pyrrolo[1,2,3-de]quinoxalin-5-yl)-7-fluoro-1-methyl-1H-benzo[d]imidazol-5-yl)methanone

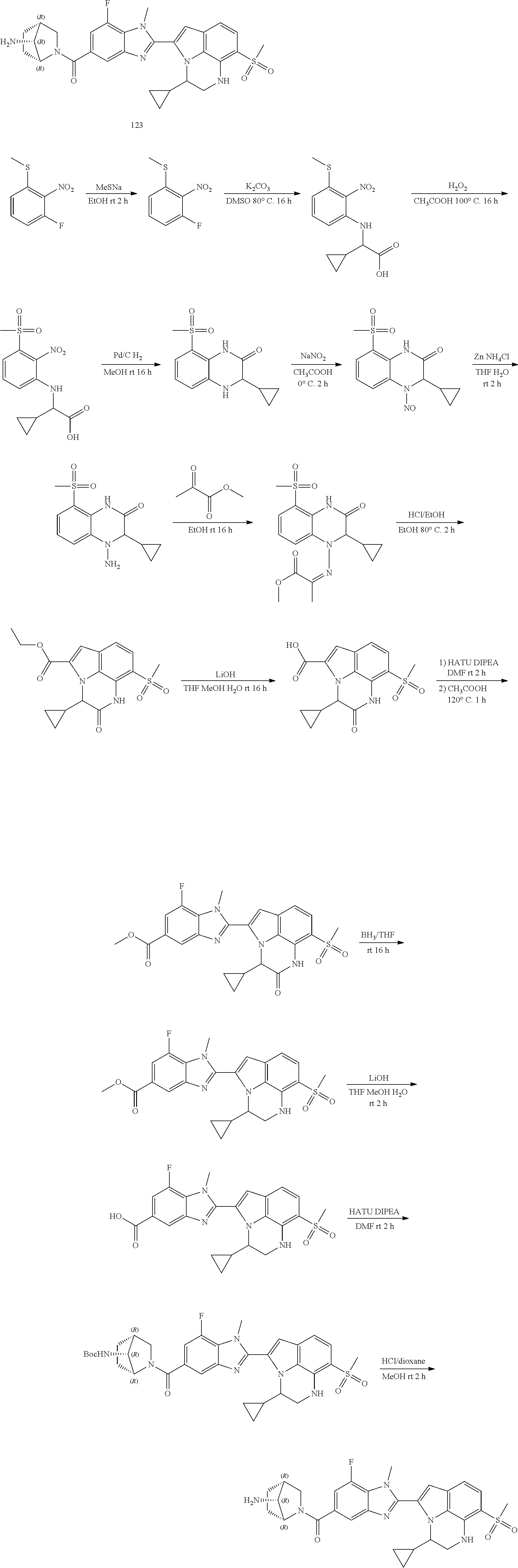

### Step 1:

To a stirred solution of 1,3-difluoro-2-nitrobenzene (10.0 g, 62.86 mmol, 6.65 mL) in EtOH (100 mL) was added sodium thiomethoxide (22.05 g, 62.86 mmol, 20 wt % in water) in portions over 5 min. The mixture was stirred at RT for 2 h, then evaporated in vacuo and purified by flash column chromatography on silica gel (eluting with 0-30% EA in PE) to give (3-fluoro-2-nitrophenyl)(methyl)sulfane (10.2 g, 54.49 mmol, 86.7% yield).

### Step 2:

A mixture of (3-fluoro-2-nitrophenyl)(methyl)sulfane (6.0 g, 32.05 mmol), 2-amino-2-cyclopropyl-acetic acid (7.38 g, 64.11 mmol) and potassium carbonate (13.29 g, 96.16 mmol) in anhydrous DMSO (100 mL) was heated at 80° C. for 16 h. After cooling to RT, the reaction mixture was carefully poured into water (100 mL) with vigorous stirring. The aq. layer was washed with methyl tert-butyl ether to remove organic impurities. The aq. layer was then acidified to pH ˜1.5 with conc. HCl to give an orange precipitation. The orange solid was collected by filtration, washed with water and air-dried to afford 2-cyclopropyl-2-((3-(methylthio)-2-nitrophenyl)amino)acetic acid (8.5 g, 30.11 mmol, 93.9% yield). LC/MS (ESI⁺): m/z 282.8 [(M+H)⁺].

### Step 3:

To a stirred mixture of 2-cyclopropyl-2-(3-methylsulfanyl-2-nitro-anilino)acetic acid (8.5 g, 30.11 mmol) in CH₃COOH (10 mL) was added H₂O₂(10 mL, >30% in water), and the resulting mixture was was heated at 1000° C. for 16 h. Upon completion, the mixture was cooled and then poured into ice water. The precipitation formed was collected by filtration, washed with water and dried in vacuo to give 2-cyclopropyl-2-((3-(methylsulfonyl)-2-nitrophenyl)amino)acetic acid (9.2 g, 29.27 mmol, 97.2% yield). LC/MS (ESI⁺): m/z 314.8 [(M+H)⁺].

### Step 4:

A mixture of 2-cyclopropyl-2-(3-methylsulfonyl-2-nitro-anilino)acetic acid (9.2 g, 29.27 mmol) and 10 wt % Pd/C (450 mg) in MeOH (100 mL) under H₂ atmosphere was stirred at RT for 16 h. Upon completion, the mixture was filtered, and the filtrate was concentrated in vacuo to afford 3-cyclopropyl-8-(methylsulfonyl)-3,4-dihydroquinoxalin-2(1H)-one (6.4 g, 24.03 mmol, 82.1% yield) as a brown solid. LC/MS (ESI⁺): m/z 266.8 [(M+H)⁺].

### Step 5:

To a stirred solution of 3-cyclopropyl-8-methylsulfonyl-3,4-dihydro-1H-quinoxalin-2-one (6.4 g, 24.03 mmol) in CH₃COOH (60 mL) was added a solution of sodium nitrite (1.66 g, 24.03 mmol) in H₂O (15 mL) at 0° C. The resulting mixture was stirred at 0° C. for 2 h. Upon completion, the mixture was diluted with DCM and washed with brine. The organic layer was dried over Na₂SO₄, filtered and concentrated in vacuo to give 3-cyclopropyl-8-(methylsulfonyl)-4-nitroso-3,4-dihydroquinoxalin-2(1H)-one (7.0 g, 23.70 mmol, 98.6% yield) as a yellow solid. LC/MS (ESI⁺): m/z 295.8 [(M+H)⁺].

### Step 6:

To a stirred solution of 3-cyclopropyl-8-methylsulfonyl-4-nitroso-1,3-dihydroquinoxalin-2-one (7.0 g, 23.70 mmol) in THF (70 mL) was added a solution of ammonium chloride (5.07 g, 94.81 mmol, 3.31 mL) in H₂O (70 mL) and then zinc powder (6.20 g, 94.81 mmol). The resulting mixture was stirred at RT for 2 h. Upon completion, the mixture was filtered and the filtrate was extracted with EtOAc (80 mL×2). The combined organic layers were dried over Na₂SO₄, filtered, and concentrated in vacuo to afford 4-amino-3-cyclopropyl-8-(methylsulfonyl)-3,4-dihydroquinoxalin-2(1H)-one (5.2 g, 18.48 mmol, 77.9% yield) as a yellow solid, which was used in the next step directly without further purification. LC/MS (ESI⁺): m/z 281.8 [(M+H)⁺].

### Step 7:

A mixture of 4-amino-3-cyclopropyl-8-methylsulfonyl-1,3-dihydroquinoxalin-2-one (5.2 g, 18.48 mmol) and ethyl 2-oxopropanoate (2.15 g, 18.48 mmol) in EtOH (50 mL) was stirred at RT under N₂ atmosphere for 16 h. Upon completion, the mixture was concentrated in vacuo, and the residue was slurried in DCM (12.5 mL, 5 v/w) and PE (62.5 mL, 25 v/w). The precipitation was collected by filtration and dried in vacuo to afford ethyl (2Z)-2-[(2-cyclopropyl-5-methylsulfonyl-3-oxo-2,4-dihydroquinoxalin-1-yl)imino]propanoate (6.2 g, 16.34 mmol, 88.4% yield) as a yellow solid. LC/MS (ESI⁺): m/z 379.8 [(M+H)⁺].

### Step 8:

To a stirred solution of ethyl (2Z)-2-[(2-cyclopropyl-5-methylsulfonyl-3-oxo-2,4-dihydroquinoxalin-1-yl)imino]propanoate (6.5 g, 17.13 mmol) in EtOH (50 mL) was added 4 M HCl in EtOH (50 mL). The resulting mixture was stirred at 80° C. for 2 h. After cooling to RT, the mixture was concentrated in vacuo. The residue was diluted with DCM, washed with water, dried over Na₂SO₄, filtered, and concentrated in vacuo. The crude product was purified by silica gel column chromatography (eluting with 0-20% EA in PE) to afford ethyl 3-cyclopropyl-9-(methylsulfonyl)-2-oxo-2,3-dihydro-1H-pyrrolo[1,2,3-de]quinoxaline-5-carboxylate (3.6 g, 9.93 mmol, 57.9% yield) as a yellow solid. LC/MS (ESI⁺): m/z 362.7 [(M+H)⁺].

### Step 9:

To a stirred solution of ethyl 11-cyclopropyl-7-methylsulfonyl-10-oxo-1,9-diazatricyclo[6.3.1.0^{4,12}]dodeca-2,4,6,8(12)-tetraene-2-carboxylate (2.0 g, 5.52 mmol) in THF/MeOH mixed solvents (30 mL, 2:1) was added LiOH aqueous solution (1.0 M, 22 mL). The mixture was stirred at RT for 16 h. Upon completion, the mixture was acidified to pH=5-6 with 3 M HCl aqueous solution, and extracted with EA (20 mL×3). The combined organic layers were dried over anhydrous sodium sulfate and filtered. The filtrate was concentrated in vacuo to give 11-cyclopropyl-7-methylsulfonyl-10-oxo-1,9-diazatricyclo[6.3.1.0^{4,12}]dodeca-2,4,6,8(12)-tetraene-2-carboxylic acid (1.5 g, 4.49 mmol, 81.3% yield) as a white solid. LC/MS (ESI⁺): m/z 334.7 [(M+H)⁺].

### Step 10:

To a stirred solution of 11-cyclopropyl-7-methylsulfonyl-10-oxo-1,9-diazatricyclo[6.3.1.0^{4,12}]dodeca-2,4,6,8(12)-tetraene-2-carboxylic acid (1.5 g, 4.49 mmol) in DMF (15 mL) were added DIPEA (1.74 g, 13.46 mmol), HATU (2.05 g, 5.38 mmol) and methyl 3-amino-5-fluoro-4-(methylamino)benzoate (889.2 mg, 4.49 mmol). The resulting mixture was stirred at RT for 2 h. Upon completion, the mixture was diluted with EA, washed with brine, and dried over anhydrous sodium sulfate. After filtration and evaporation of the solvent in vacuo, the residue was stirred in CH₃COOH (20 mL) at 120° C. for 1 h. After cooling to RT, the mixture was diluted with EA (80 mL), washed with saturated Na₂CO₃ solution and dried over Na₂SO₄. After filtration and evaporation of the solvent in vacuo, the residue was purified by flash column chromatography on silica gel (eluting with 0-7% MeOH in DCM) to give methyl 2-(3-cyclopropyl-9-(methylsulfonyl)-2-oxo-2,3-dihydro-1H-pyrrolo[1,2,3-de]quinoxalin-5-yl)-7-fluoro-1-methyl-1H-benzo[d]imidazole-5-carboxylate (0.9 g, 1.81 mmol, 40.4% yield) as a yellow solid. LC/MS (ESI⁺): m/z 496.6 [(M+H)⁺].

### Step 11:

To a stirred solution of methyl 2-(11-cyclopropyl-7-methylsulfonyl-10-oxo-1,9-diazatricyclo[6.3.1.0^{4,12}]dodeca-2,4,6,8(12)-tetraen-2-yl)-7-fluoro-1-methyl-benzimidazole-5-carboxylate (200 mg, 402.81 μmol) in anhydrous THF (2 mL) was dropwise added borane tetrahydrofuran (1 M, 1.6 mL) at 0° C. The reaction mixture was stirred at RT for 16 h. Upon completion, the mixture was quenched with MeOH at 0° C. and concentrated in vacuo. The residue was diluted with 2 M HCl aqueous solution (3 mL) and stirred at RT for 1 h. The mixture was then basified with 4 M NaOH aqueous solution to pH ˜8 and extracted with DCM (30 mL×3). The combined organic layers were dried over anhydrous sodium sulfate and filtered. The filtrate was concentrated in vacuo and the residue was purified by flash column chromatography on silica gel (eluting with 0-5% MeOH in DCM) to give methyl 2-(11-cyclopropyl-7-methylsulfonyl-1,9-diazatricyclo[6.3.1.0^{4,12}]dodeca-2,4,6,8(12)-tetraen-2-yl)-7-fluoro-1-methyl-benzimidazole-5-carboxylate (80 mg, 165.79 μmol, 41.2% yield) as a yellow solid. LC/MS (ESI⁺): m/z 482.6 [(M+H)⁺].

### Step 12:

To a stirred solution of methyl 2-(11-cyclopropyl-7-methylsulfonyl-1,9-diazatricyclo[6.3.1.0^{4,12}]dodeca-2,4,6,8(12)-tetraen-2-yl)-7-fluoro-1-methyl-benzimidazole-5-carboxylate (80 mg, 165.79 μmol) in THF/MeOH mixed solvents (3 mL, 2:1) was added LiOH aqueous solution (1.0 M, 0.66 mL) and the mixture was stirred at RT for 2 h. Upon completion, the mixture was acidified to pH=5-6 with 3 M hydrochloric acid aqueous solution and extracted with DCM (20 mL×3). The combined organic layers were dried over anhydrous sodium sulfate and filtered. The filtrate was concentrated in vacuo to give 2-(11-cyclopropyl-7-methylsulfonyl-1,9-diazatricyclo[6.3.1.0^{4,12}]dodeca-2,4,6,8(12)-tetraen-2-yl)-7-fluoro-1-methyl-benzimidazole-5-carboxylic acid (75 mg, 160.09 μmol, 96.6% yield) as a white solid. LC/MS (ESI⁺): m/z 468.6 [(M+H)⁺].

### Step 13:

To a stirred solution 2-(11-cyclopropyl-7-methylsulfonyl-1,9-diazatricyclo[6.3.1.0^{4,12}]dodeca-2,4,6,8(12)-tetraen-2-yl)-7-fluoro-1-methyl-benzimidazole-5-carboxylic acid (75 mg, 160.09 μmol) in DMF (2 mL) were added DIPEA (62.1 mg, 480.26 μmol), HATU (73.0 mg, 192.10 μmol), and tert-butyl ((1R,4R,7R)-2-azabicyclo[2.2.1]heptan-7-yl)carbamate (34.0 mg, 160.09 μmol). The resulting mixture was stirred at RT for 2 h. Upon completion, the mixture was diluted with EA, washed with brine, and dried over anhydrous sodium sulfate. After filtration and evaporation of the solvent in vacuo, the residue was purified by flash column chromatography on silica gel (eluting with 0-8% MeOH in DCM) to give tert-butyl ((1R,4R,7R)-2-(2-(3-cyclopropyl-9-(methylsulfonyl)-2,3-dihydro-1H-pyrrolo[1,2,3-de]quinoxalin-5-yl)-7-fluoro-1-methyl-1H-benzo[d]imidazole-5-carbonyl)-2-azabicyclo[2.2.1]heptan-7-yl)carbamate (50 mg, 75.44 μmol, 47.1% yield) as a white solid. LC/MS (ESI⁺): m/z 662.6 [(M+H)⁺].

### Step 14:

To a stirred solution tert-butyl N-[(1R,4R,7R)-2-[2-(11-cyclopropyl-7-methylsulfonyl-1,9-diazatricyclo[6.3.1.0^{4,12}]dodeca-2,4,6,8(12)-tetraen-2-yl)-7-fluoro-1-methyl-benzimidazole-5-carbonyl]-2-azabicyclo[2.2.1]heptan-7-yl]carbamate (50 mg, 75.44 μmol) in MeOH (2 mL) was added 4 M HCl in dioxane (2 mL). The resulting mixture was stirred at RT for 2 h. Upon completion, the mixture was concentrated in vacuo, basified with saturated Na₂CO₃ solution to pH=8 and extracted with DCM (30 mL×3). The combined organic layers were dried over anhydrous sodium sulfate. After filtration and evaporation of the solvent in vacuo, the residue was purified by flash column chromatography on silica gel (eluting with 0-10% MeOH in DCM) to give [(1R,4R,7R)-7-amino-2-azabicyclo[2.2.1]heptan-2-yl]-[2-(11-cyclopropyl-7-methylsulfonyl-1,9-diazatricyclo[6.3.1.0^{4,12}]dodeca-2,4,6,8(12)-tetraen-2-yl)-7-fluoro-1-methyl-benzimidazol-5-yl]methanone (33 mg, 58.65 μmol, 77.7% yield) as a white solid. LC/MS (ESI⁺): m/z 562.7 [(M+H)⁺]. ¹H NMR (400 MHz, DMSO-d₆) δ 7.73-7.59 (m, 1H), 7.27-7.24 (m, 1H), 7.12 (d, J=1.5 Hz, 1H), 7.05 (d, J=8.6 Hz, 1H), 6.88 (d, J=2.9 Hz, 1H), 4.65-4.56 (m, 1H), 4.13 (t, J=2.5 Hz, 3H), 3.87-3.73 (m, 2H), 3.73-3.59 (m, 1H), 3.55-3.48 (m, 1H), 3.20 (s, 1H), 3.16 (s, 3H), 3.12-2.98 (m, 2H), 2.22 (t, J=3.7 Hz, 1H), 2.05-1.85 (m, 2H), 1.79-1.63 (m, 1H), 1.50-1.36 (m, 1H), 1.23 (s, 1H), 0.97 (tq, J=8.9, 4.3 Hz, 1H), 0.36 (tt, J=8.6, 4.7 Hz, 2H), 0.10 (p, J=4.5 Hz, 1H), −0.72 (dh, J=14.3, 4.8 Hz, 1H).

### Example 124 Synthesis report of 5-(5-((1R,4R,7R)-7-amino-2-azabicyclo[2.2.1]heptane-2-carbonyl)-7-fluoro-1-methyl-1H-benzo[d]imidazol-2-yl)-9-chloro-3-ethyl-1H-pyrrolo[1,2,3-de]quinoxalin-2(3H)-one

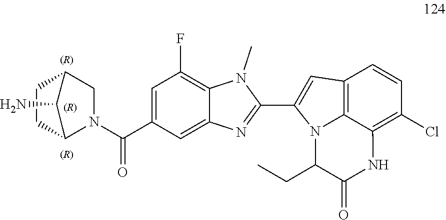

Prepared in analogous manner as for Example 114. LC/MS (ESI⁺): m/z 520.7 [(M+H)⁺]. ¹H NMR (400 MHz, DMSO-d₆) δ 11.09 (d, J=3.0 Hz, 1H), 8.62 (s, 1H), 8.47 (s, 1H), 7.88 (s, 1H), 7.75 (d, J=4.3 Hz, 1H), 7.40 (d, J=8.0 Hz, 2H), 7.15 (d, J=8.5 Hz, 1H), 6.03 (dd, J=5.9, 3.3 Hz, 1H), 4.26 (s, 3H), 3.63 (s, 2H), 3.29-3.20 (m, 1H), 2.74 (s, 1H), 2.06 (d, J=16.1 Hz, 3H), 1.66 (d, J=14.2 Hz, 2H), 1.30 (s, 6H).

### Example 125 Synthesis report of ((1R,4R,7R)-7-amino-2-azabicyclo[2.2.1]heptan-2-yl)(2-(9-chloro-3-ethyl-2,3-dihydro-1H-pyrrolo[1,2,3-de]quinoxalin-5-yl)-7-fluoro-1-methyl-1H-benzo[d]imidazol-5-yl)methanone

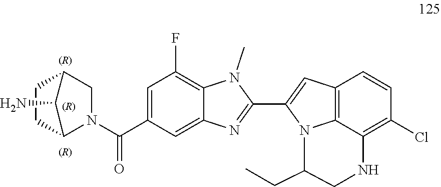

Prepared in analogous manner as for Example 114. LC/MS (ESI⁺): m/z 506.8 [(M+H)⁺].

### Example 126 Synthesis report of 5-(5-((1R,4R,7R)-7-amino-2-azabicyclo[2.2.1]heptane-2-carbonyl)-7-fluoro-1-methyl-1H-benzo[d]imidazol-2-yl)-3-ethyl-9-fluoro-1H-pyrrolo[1,2,3-de]quinoxalin-2(3H)-one

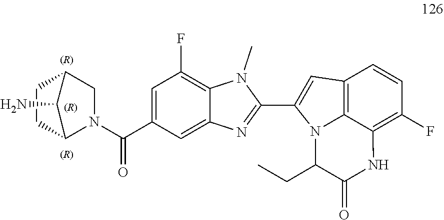

Prepared in analogous manner as for Example 114. LC/MS (ESI⁺): m/z 504.8 [(M+H)⁺].

### Example 127 Synthesis of 5-(5-((1R,4R,7R)-7-amino-2-azabicyclo[2.2.1]heptane-2-carbonyl)-7-fluoro-1-methyl-1H-benzo[d]imidazol-2-yl)-3-ethyl-2-oxo-2,3-dihydro-1H-pyrrolo[1,2,3-de]quinoxaline-9-carbonitrile

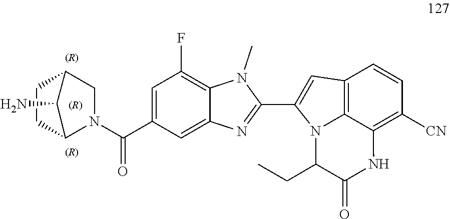

Prepared in analogous manner as for Example 114. LC/MS (ESI⁺): m/z 511.7 [(M+H)⁺]. ¹H NMR (400 MHz, DMSO-d₆) δ 7.67 (dd, J=2.5, 1.2 Hz, 1H), 7.45-7.35 (m, 2H), 7.34-7.23 (m, 2H), 5.99 (td, J=5.9, 3.2 Hz, 1H), 4.20 (d, J=2.0 Hz, 3H), 3.87 (d, J=10.1 Hz, 1H), 3.69 (dtdd, J=11.0,6.4, 4.4, 1.9 Hz, 2H), 3.57 (s, 1H), 3.57-3.42 (m, 5H), 3.32 (s, 2H), 3.16-3.05 (m, 2H), 2.35 (d, J=8.8 Hz, 1H), 2.04-1.84 (m, 3H), 1.84-1.72 (m, 1H), 1.64 (ddt, J=14.1, 11.9, 7.1 Hz, 1H), 1.49 (t, J=8.3 Hz, 1H), 1.38-1.20 (m, 1H), 0.51-0.41 (m, 3H).

### Example 128 Synthesis of 5-(5-((1R,4R,7R)-7-amino-2-azabicyclo[2.2.1]heptane-2-carbonyl)-7-fluoro-1-methyl-1H-benzo[d]imidazol-2-yl)-3-ethyl-2,3-dihydro-1H-pyrrolo[1,2,3-de]quinoxaline-9-carbonitrile

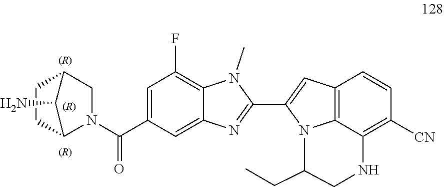

Prepared in analogous manner as for Example 114. LC/MS (ESI⁺): m/z 497.8 [(M+H)⁺]. ¹H NMR (400 MHz, DMSO-d₆) δ 7.61 (dd, J=4.2, 1.2 Hz, 1H), 7.35-7.20 (m, 1H), 7.15 (s, 2H), 7.05 (d, J=8.4 Hz, 1H), 6.99 (d, J=8.4 Hz, 1H), 5.28 (dq, J=7.0, 3.5 Hz, 1H), 4.17 (d, J=3.5 Hz, 3H), 3.77-3.60 (m, 3H), 3.51 (dt, J=10.9, 2.8 Hz, 1H), 3.21 (s, 1H), 3.12-2.98 (m, 1H), 2.22 (d, J=3.8 Hz, 1H), 1.96 (qd, J=16.0, 9.1 Hz, 2H), 1.77-1.63 (m, 1H), 1.58 (dt, J=7.3, 3.7 Hz, 1H), 1.54 (dd, J=7.4, 3.4 Hz, 1H), 1.43 (dd, J=9.8, 7.2 Hz, 1H), 1.41-1.24 (m, 1H), 1.24 (s, 1H), 0.66 (td, J=7.4, 3.4 Hz, 3H).

### Example 129 Synthesis report of 5-(5-((1R,4R,7R)-7-amino-2-azabicyclo[2.2.1]heptane-2-carbonyl)-7-fluoro-1-methyl-1H-benzo[d]imidazol-2-yl)-3-ethyl-1H-pyrrolo[1,2,3-de]quinoxalin-2(3H)-one

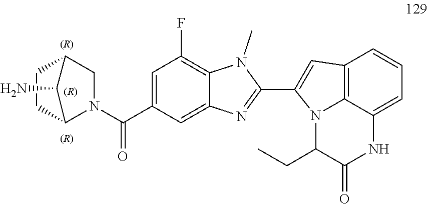

Prepared in analogous manner as for Example 114. LC/MS (ESI⁺): m/z 486.8 [(M+H)⁺]. ¹H NMR (400 MHz, DMSO-d₆) δ 11.08 (s, 1H), 8.21 (s, 1H), 8.01-7.73 (m, 1H), 7.64 (dd, J=3.6, 1.2 Hz, 1H), 7.34-7.29 (m, 2H), 7.24 (dt, J=12.1, 1.5 Hz, 1H), 7.05 (t, J=7.7 Hz, 1H), 6.70 (d, J=7.3 Hz, 1H), 5.94 (dt, J=6.3, 3.4 Hz, 1H), 4.21 (d, J=2.3 Hz, 3H), 3.80 (d, J=11.6 Hz, 1H), 3.68 (d, J=9.0 Hz, 1H), 3.11 (d, J=11.2 Hz, 1H), 2.91 (d, J=11.0 Hz, 1H), 2.74 (s, 1H), 2.29 (s, 1H), 2.03-1.82 (m, 3H), 1.57 (dd, J=9.1, 4.3 Hz, 1H), 1.50-1.41 (m, 1H), 0.54-0.35 (m, 3H).

### Example 130 Synthesis of ((1R,4R,7R)-7-amino-2-azabicyclo[2.2.1]heptan-2-yl)(2-(3-ethyl-8-fluoro-2,3-dihydro-1H-pyrrolo[1,2,3-de]quinoxalin-5-yl)-7-fluoro-1-methyl-1H-benzo[d]imidazol-5-yl)methanone

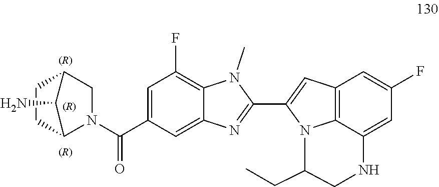

Prepared in analogous manner as for Example 114. LC/MS (ESI⁺): m/z 490.7 [(M+H)⁺]. ¹H NMR (400 MHz, DMSO-d₆) δ 7.59 (dd, J=4.1, 1.3 Hz, 1H), 7.36-7.17 (m, 1H), 7.00 (d, J=2.7 Hz, 1H), 6.64 (dd, J=10.2, 2.1 Hz, 1H), 6.44 (d, J=2.8 Hz, 1H), 6.21 (dd, J=11.2, 2.2 Hz, 1H), 5.21 (d, J=7.4 Hz, 1H), 4.16 (d, J=3.4 Hz, 3H), 3.75-3.61 (m, 1H), 3.54 (dtt, J=13.5, 8.0, 2.8 Hz, 3H), 3.18 (s, 1H), 3.15-2.97 (m, 1H), 2.04-1.87 (m, 2H), 1.78-1.65 (m, 2H), 1.63-1.49 (m, 2H), 1.49-1.35 (m, 1H), 0.66 (td, J=7.4, 3.3 Hz, 3H).

### Example 131 Synthesis report of ((1R,4R,7R)-7-amino-2-azabicyclo[2.2.1]heptan-2-yl)(2-(3-ethyl-9-fluoro-2,3-dihydro-1H-pyrrolo[1,2,3-de]quinoxalin-5-yl)-7-fluoro-1-methyl-1H-benzo[d]imidazol-5-yl)methanone

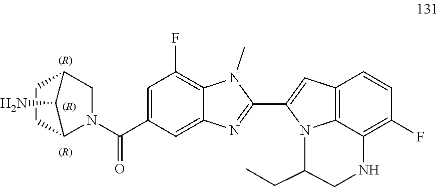

Prepared in analogous manner as for Example 114. LC/MS (ESI⁺): m/z 490.7 [(M+H)⁺]. ¹H NMR (400 MHz, DMSO-d₆) δ 7.71-7.58 (m, 1H), 7.30-7.19 (m, 1H), 7.06 (d, J=2.7 Hz, 1H), 6.93-6.85 (m, 2H), 6.15 (s, 1H), 5.25 (d, J=7.3 Hz, 1H), 4.16 (d, J=3.7 Hz, 3H), 3.74 (d, J=13.4 Hz, 1H), 3.57 (s, 1H), 3.50 (td, J=8.6, 3.9 Hz, 3H), 3.21 (s, 1H), 3.08 (d, J=11.0 Hz, 1H), 2.25-2.15 (m, 1H), 1.94 (d, J=9.8 Hz, 2H), 1.79-1.69 (m, 1H), 1.59 (pd, J=7.4, 3.7 Hz, 2H), 1.43 (dd, J=14.4, 5.0 Hz, 1H), 0.67 (td, J=7.4, 3.1 Hz, 3H).

### Example 132 Synthesis of ((1R,4R,7R)-7-amino-2-azabicyclo[2.2.1]heptan-2-yl)(2-(3-ethyl-9-methyl-2,3-dihydro-1H-pyrrolo[1,2,3-de]quinoxalin-5-yl)-7-fluoro-1-methyl-1H-benzo[d]imidazol-5-yl)methanone

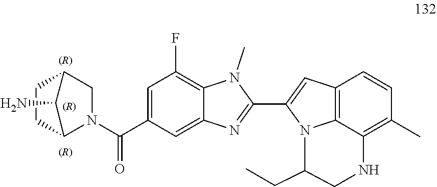

Prepared in analogous manner as for Example 114. LC/MS (ESI⁺): m/z 486.8 [(M+H)⁺]. ¹H NMR (400 MHz, DMSO-d₆) δ 7.58 (dd, J=4.9, 1.2 Hz, 1H), 7.23-7.16 (m, 1H), 6.98 (s, 1H), 6.87 (d, J=8.0 Hz, 1H), 6.77 (d, J=8.1 Hz, 1H), 5.69 (d, J=3.0 Hz, 1H), 5.33 (dd, J=5.4, 4.0 Hz, 1H), 5.23 (s, 1H), 4.16 (d, J=3.5 Hz, 3H), 3.76 (d, J=14.4 Hz, 1H), 3.63 (d, J=16.0 Hz, 1H), 3.58 (s, 1H), 3.56-3.48 (m, 2H), 3.22 (s, 1H), 3.12-3.00 (m, 2H), 2.22 (s, 3H), 2.01 (q, J=7.0 Hz, 2H), 1.96 (s, 1H), 1.74 (t, J=9.3 Hz, 1H), 1.57 (td, J=7.4, 3.8 Hz, 2H), 1.45 (q, J=8.7 Hz, 2H), 1.32 (d, J=14.9 Hz, 1H), 0.90-0.82 (m, 2H), 0.67 (td, J=7.4, 3.8 Hz, 3H).

### Example 133 Synthesis report of cis-((1R,4R,7R)-7-amino-2-azabicyclo[2.2.1]heptan-2-yl)(2-(3-ethyl-2-methyl-2,3-dihydro-1H-pyrrolo[1,2,3-de]quinoxalin-5-yl)-7-fluoro-1-methyl-1H-benzo[d]imidazol-5-yl)methanone

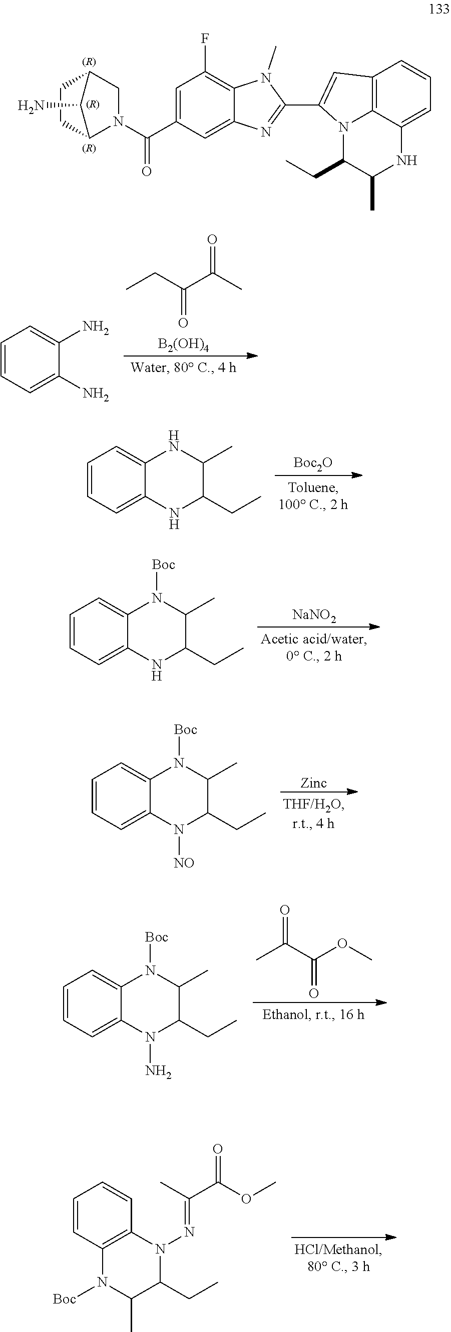

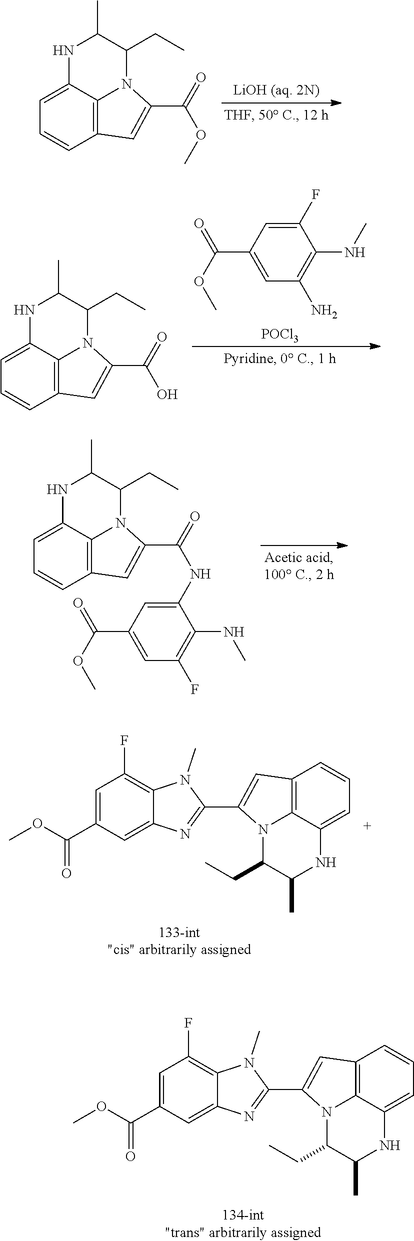

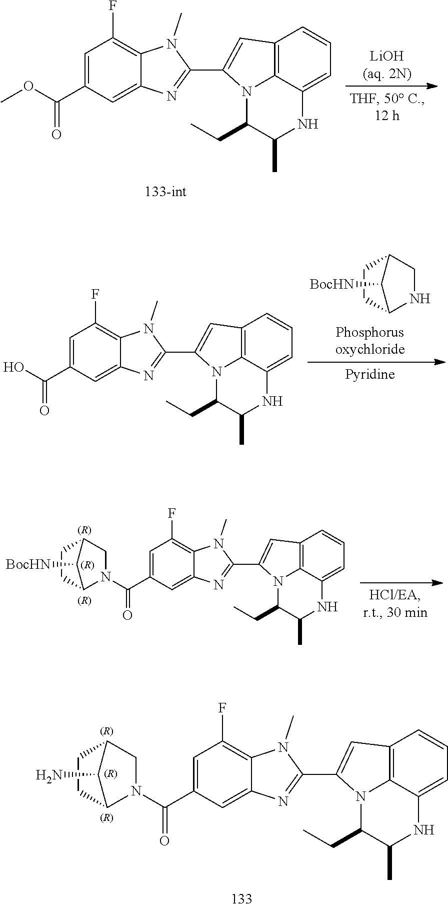

### Step 1:

A mixture of benzene-1,2-diamine (10 g, 92.47 mmol), pentane-2,3-dione (9.26 g, 92.47 mmol) and diboronic acid (34.05 g, 462.36 mmol) in water (150 mL) was stirred at 80° C. for 4 h. After cooling to RT, the mixture was extracted EtOAc (3×100 mL). The combined organic layers were washed with water, dried over anhydrous Na₂SO₄, filtered, and concentrated in vacuo. The residue was purified by flash column chromatography on silica gel (eluting with 0-20% EA in PE) to give 2-ethyl-3-methyl-1,2,3,4-tetrahydroquinoxaline (14 g, 79.43 mmol, 85.9% yield) as a yellow oil. LC/MS (ESI⁺): m/z 176.8 [(M+H)⁺].

### Step 2:

A mixture of 2-ethyl-3-methyl-1,2,3,4-tetrahydroquinoxaline (14 g, 79.43 mmol) and di-tert-butyl dicarbonate (17.34 g, 79.43 mmol) in toluene (100 mL) was stirred at 100° C. for 2 h. After cooling to RT, the mixture was concentrated in vacuo and the residue was purified by flash column chromatography on silica gel (eluting with 0-30% EA in PE) to give tert-butyl 3-ethyl-2-methyl-3,4-dihydro-2H-quinoxaline-1-carboxylate (16 g, 57.89 mmol, 72.8% yield) as a yellow oil. LC/MS (ESI⁺): m/z 220.9 [(M+H-56)⁺].

### Step 3:

To a mixture of tert-butyl 3-ethyl-2-methyl-3,4-dihydro-2H-quinoxaline-1-carboxylate (16 g, 57.89 mmol) in water (10 mL) was added a solution of sodium nitrite (3.99 g, 57.89 mmol) in water (5 mL) followed by glacial acetic acid (20 mL) at 0° C., and the resulting mixture was stirred at 0° C. for 2 h. Upon completion, the mixture was filtered, and the filter cake was dried in vacuo to give the tert-butyl 3-ethyl-2-methyl-4-nitroso-2,3-dihydroquinoxaline-1-carboxylate (12 g, 39.30 mmol, 67.8% yield) as a yellow solid. LC/MS (ESI⁺): m/z 249.9 [(M+H-56)⁺].

### Step 4:

To a mixture of tert-butyl 3-ethyl-2-methyl-4-nitroso-2,3-dihydroquinoxaline-1-carboxylate (12 g, 39.30 mmol) and ammonium chloride (6.31 g, 117.89 mmol) in H₂O/THF mixed solvents (35 mL, 1:6) was added zinc powder (7.66 g, 117.89 mmol) in portions. The mixture was stirred at RT for 4 h, then filtered, and the filter cake was washed with THF (3×30 mL). The filtrate was concentrated in vacuo and the residue was purified by flash column chromatography on silica gel (eluting with 0-50% EA in PE) to give tert-butyl 4-amino-3-ethyl-2-methyl-2,3-dihydroquinoxaline-1-carboxylate (6 g, 20.59 mmol, 52.4% yield) as a white solid. LC/MS (ESI⁺): m/z 291.8 [(M+H)⁺].

### Step 5:

A mixture of tert-butyl 4-amino-3-ethyl-2-methyl-2,3-dihydroquinoxaline-1-carboxylate (6 g, 20.59 mmol) and methyl 2-oxopropanoate (2.10 g, 20.59 mmol) in ethanol (30 mL) was stirred at RT for 16 h. Upon completion, the mixture was concentrated in vacuo to give the crude product tert-butyl 3-ethyl-4-[(E)-(2-methoxy-1-methyl-2-oxo-ethylidene) amino]-2-methyl-2,3-dihydroquinoxaline-1-carboxylate (7 g) as a yellow solid, which was used in the next step without further purification. LC/MS (ESI⁺): m/z 397.8 [(M+23)⁺].

Step 6: To a suspension of tert-butyl 3-ethyl-4-[(E)-(2-methoxy-1-methyl-2-oxo-ethylidene)amino]-2-methyl-2,3-dihydroquinoxaline-1-carboxylate (7 g, 18.64 mmol) in methanol (10 mL) was added a solution of HCl in methanol (4 M, 10 mL). The mixture was heated to 80° C. for 3 h. After cooling to RT, the mixture was concentrated in vacuo and the residue was purified by flash column chromatography on silica gel (eluting with 0-50% EA in PE) to give methyl 11-ethyl-10-methyl-1,9-diazatricyclo[6.3.1.04,12]dodeca-2,4(12), 5,7-tetraene-2-carboxylate (200 mg, 774.25 μmol, 4.1% yield) as a white solid. LC/MS (ESI⁺): m/z 258.8 [(M+H)⁺].

### Step 7:

To a mixture of methyl 11-ethyl-10-methyl-1,9-diazatricyclo[6.3.1.04,12]dodeca-2,4(12), 5,7-tetraene-2-carboxylate (200 mg, 774.25 μmol) in THF (5 mL) was added a solution of LiOH (2 M, 5 mL) and the resulting mixture was stirred at 50° C. overnight. 2 M HCl was added to make pH<7, then the mixture was concentrated in vacuo and the residue was purified by prep-HPLC to give 11-ethyl-10-methyl-1,9-diazatricyclo[6.3.1.04,12]dodeca-2,4(12), 5,7-tetraene-2-carboxylic acid (150 mg, 614.03 μmol, 79.3% yield) as a yellow solid. LC/MS (ESI⁺): m/z 244.8 [(M+H)⁺].

### Step 8:

To a stirred solution of 11-ethyl-10-methyl-1,9-diazatricyclo[6.3.1.04,12]dodeca-2,4(12), 5,7-tetraene-2-carboxylic acid (150 mg, 614.03 μmol) and methyl 3-amino-5-fluoro-4-(methylamino)benzoate (121.70 mg, 614.03 μmol) in pyridine (5 mL) at 0° C. was added POCl₃ (188.30 mg, 1.23 mmol) dropwise. The resulting mixture was stirred at RT for 30 min. About 1 mL of water was added into the mixture to quench the reaction. Then solvent was removed in vacuo and the residue was purified by flash column chromatography on silica gel (eluting with 0-50% EA in PE) to give methyl 3-[(11-ethyl-10-methyl-1,9-diazatricyclo[6.3.1.04,12]dodeca-2,4(12), 5,7-tetraene-2-carbonyl)amino]-5-fluoro-4-(methylamino)benzoate (50 mg, 117.79 μmol, 19.1% yield) as a white solid. LC/MS (ESI⁺): m/z 424.8 [(M+H)⁺].

### Step 9:

A mixture of methyl 3-[(11-ethyl-10-methyl-1,9-diazatricyclo[6.3.1.04,12]dodeca-2,4(12), 5,7-tetraene-2-carbonyl)amino]-5-fluoro-4-(methylamino)benzoate (50 mg, 117.79 μmol) in acetic acid (5 mL) was stirred at 100° C. for 2 h. After cooling to RT, solvent was removed in vacuo. The residue was purified by flash column chromatography on silica gel (eluting with 0-50% EA in PE) to give assumed cis-product 133-int (first elute, 10 mg, 24.60 μmol, 20.8% yield, cis configuration is assigned arbitrarily) and assumed trans-product 134-int (second elute, 6 mg, 14.76 μmol, 12.5% yield, trans configuration is assigned arbitrarily). LC/MS (ESI⁺): m/z 406.8 [(M+H)⁺].

### Step 10:

To a mixture of cis-methyl 2-(3-ethyl-2-methyl-2,3-dihydro-1H-pyrrolo[1,2,3-de]quinoxalin-5-yl)-7-fluoro-1-methyl-1H-benzo[d]imidazole-5-carboxylate 133-int (10 mg, 24.60 μmol) in THF (1 mL) was added a solution of LiOH (2 M, 1 mL). The mixture was stirred at 50° C. overnight. 2 M HCl was added to acidify the mixture. Then the mixture was concentrated in vacuo and the residue was purified by prep-HPLC to give cis-2-(3-ethyl-2-methyl-2,3-dihydro-1H-pyrrolo[1,2,3-de]quinoxalin-5-yl)-7-fluoro-1-methyl-1H-benzo[d]imidazole-5-carboxylic acid (6 mg, 15.29 μmol, 62.1% yield) as a yellow solid. LC/MS (ESI⁺): m/z 392.7 [(M+H)⁺].

### Step 11:

To a stirred solution of cis-2-(3-ethyl-2-methyl-2,3-dihydro-1H-pyrrolo[1,2,3-de]quinoxalin-5-yl)-7-fluoro-1-methyl-1H-benzo[d]imidazole-5-carboxylic acid (6 mg, 15.29 μmol) and tert-butyl N—[(1R,4R,7R)-2-azabicyclo[2.2.1]heptan-7-yl]carbamate (3.25 mg, 15.29 μmol) in pyridine (2 mL) at 0° C. was added POCl₃ (2.81 mg, 18.35 μmol) dropwise. The resulting mixture was stirred at RT for 30 min. About 1 mL of water was added into the mixture to quench the reaction. Solvent was removed and the residue was purified by flash column chromatography on silica gel (eluting with 0-50% EA in PE) to give cis-tert-butyl ((1R,4R,7R)-2-(2-(3-ethyl-2-methyl-2,3-dihydro-1H-pyrrolo[1,2,3-de]quinoxalin-5-yl)-7-fluoro-1-methyl-1H-benzo[d]imidazole-5-carbonyl)-2-azabicyclo[2.2.1]heptan-7-yl)carbamate (3 mg, 5.11 μmol, 33.4% yield) as a white solid. LC/MS (ESI⁺): m/z 586.8 [(M+H)⁺].

### Step 12:

To a stirred mixture of cis-tert-butyl ((1R,4R,7R)-2-(2-(3-ethyl-2-methyl-2,3-dihydro-1H-pyrrolo[1,2,3-de]quinoxalin-5-yl)-7-fluoro-1-methyl-1H-benzo[d]imidazole-5-carbonyl)-2-azabicyclo[2.2.1]heptan-7-yl)carbamate (3 mg, 5.11 μmol) in EA (0.5 mL) was added 4 M HCl in EA (1 mL). The mixture was stirred at RT for 30 min. Solvent was removed in vacuo and the residue was purified by prep-HPLC to afford cis-((1R,4R,7R)-7-amino-2-azabicyclo[2.2.1]heptan-2-yl)(2-(3-ethyl-2-methyl-2,3-dihydro-1H-pyrrolo[1,2,3-de]quinoxalin-5-yl)-7-fluoro-1-methyl-1H-benzo[d]imidazol-5-yl)methanone (2 mg, 4.11 μmol, 80.3% yield) as a yellow solid. LC/MS (ESI⁺): m/z 486.8 [(M+H)⁺].

### Example 134 Synthesis report of trans-((1R,4R,7R)-7-amino-2-azabicyclo[2.2.1]heptan-2-yl)(2-(3-ethyl-2-methyl-2,3-dihydro-1H-pyrrolo[1,2,3-de]quinoxalin-5-yl)-7-fluoro-1-methyl-1H-benzo[d]imidazol-5-yl)methanone

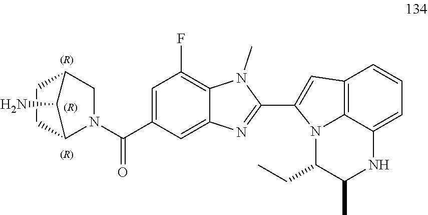

Prepared in analogous manner as for Example 133. LC/MS (ESI⁺): m/z 486.8 [(M+H)⁺].

### Example 135 Synthesis of ((1R,4R,7R)-7-amino-2-azabicyclo[2.2.1]heptan-2-yl)(2-(3-ethyl-4,5-dihydro-3H-2a, 5,6-triazaacenaphthylen-2-yl)-7-fluoro-1-methyl-1H-benzo[d]imidazol-5-yl)methanone

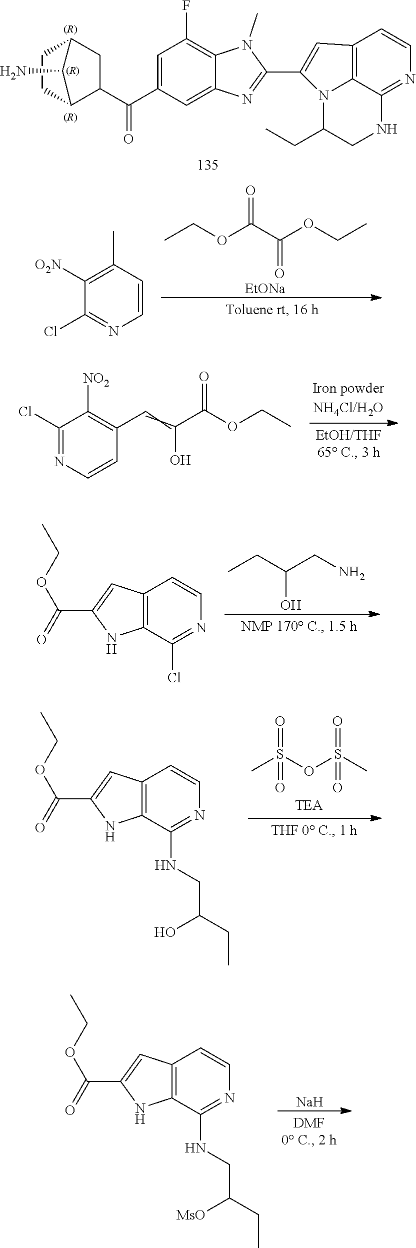

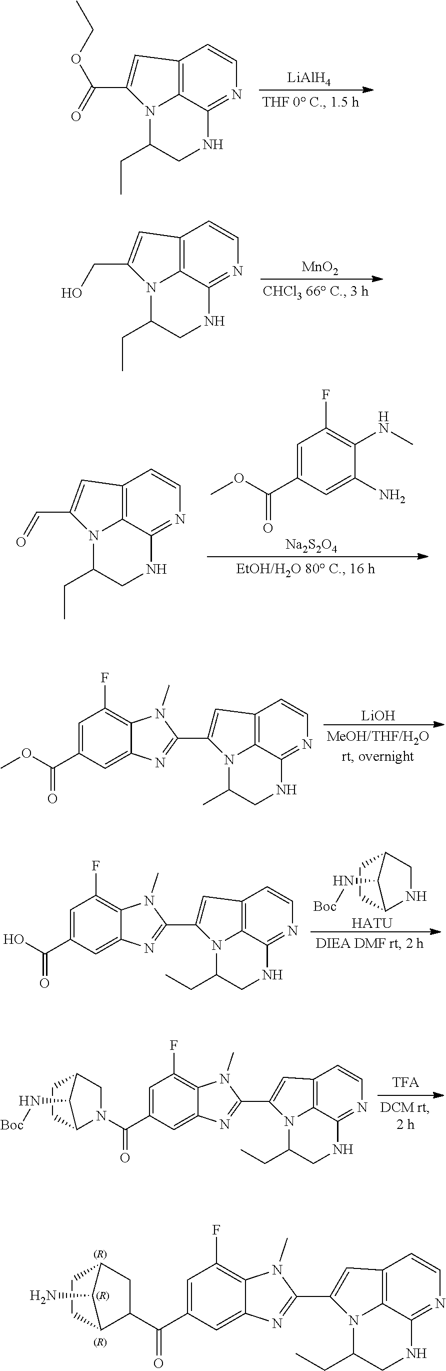

### Step 1:

To a mixture of diethyl oxalate (1.27 g, 8.69 mmol) in toluene (5 mL) was added sodium ethoxide (591.51 mg, 8.69 mmol, 20% in ethanol) at RT and the resulting mixture was stirred at RT for 10 min. Then 2-chloro-4-methyl-3-nitro-pyridine (1.0 g, 5.79 mmol) was added, and the mixture was stirred at RT for 16 h. Upon completion, the mixture was concentrated in vacuo. The residue was diluted with water (20 ml) and acidified with acetic acid (10 ml) to pH=4. The precipitation formed was collected by filtration and dried to give ethyl 3-(2-chloro-3-nitro-4-pyridyl)-2-hydroxy-prop-2-enoate (1.33 g, 4.88 mmol, 84.1% yield) as a yellow solid. LC/MS (ESI⁺): m/z 272.8 [(M+H)⁺].

### Step 2:

To a mixture of ethyl 3-(2-chloro-3-nitro-4-pyridyl)-2-hydroxy-prop-2-enoate (1.33 g, 4.88 mmol) in EtOH/THF mixed solvents (15 mL, 1:2) was added iron powder (1.36 g, 24.39 mmol) and ammonium chloride (2.09 g, 39.03 mmol) in water (4 mL). The resulting mixture was stirred at 65° C. for 3 h, then filtered through a celite bed. The filtrate was diluted with water, then basified with saturated NaHCO₃ solution, and extracted with EA (2×100 ml). The combined organic layers were dried over Na₂SO₄, filtered, concentrated in vacuo and purified by flash chromatography on silica gel (eluting with 0-50% EA in PE) to give ethyl 7-chloro-1H-pyrrolo[2,3-c]pyridine-2-carboxylate (285 mg, 1.27 mmol, 26.0% yield). LC/MS (ESI⁺): m/z 224.8 [(M+H)⁺].

### Step 3:

A mixture of ethyl 7-chloro-1H-pyrrolo[2,3-c]pyridine-2-carboxylate (1.0 g, 4.45 mmol) and 1-aminobutan-2-ol (991.97 mg, 11.13 mmol) in NMP (10 mL) was stirred at 170° C. for 1.5 h with microwave irradiation. After cooling to RT, the mixture was diluted with water (10 mL) and extracted with EA (3×15 mL). The combined organic layers were washed with brine, dried over Na₂SO₄, filtered, and concentrated in vacuo. The residue was purified by flash chromatography (eluting with 0-20% MeOH in DCM) to give ethyl 7-(2-hydroxybutylamino)-1H-pyrrolo[2,3-c]pyridine-2-carboxylate (320 mg, 1.15 mmol, 25.9% yield) as a yellow solid. LC/MS (ESI⁺): m/z 277.8 [(M+H)⁺].

### Step 4:

To a mixture of ethyl 7-(2-hydroxybutylamino)-1H-pyrrolo[2,3-c]pyridine-2-carboxylate (300 mg, 1.08 mmol) in THF (5 mL) were added TEA (547.33 mg, 5.41 mmol) and methanesulfonic anhydride (207.29 mg, 1.19 mmol) at 0° C. The mixture was stirred at 0° C. for 1 h. Then the mixture was concentrated in vacuo to give crude ethyl 7-(2-methylsulfonyloxybutylamino)-1H-pyrrolo[2,3-c]pyridine-2-carboxylate (380 mg, 1.07 mmol, 98.8% yield) as a yellow oil. LC/MS (ESI⁺): m/z 355.7 [(M+H)⁺].

### Step 5:

To a mixture of ethyl 7-(2-methylsulfonyloxybutylamino)-1H-pyrrolo[2,3-c]pyridine-2-carboxylate (380 mg, 1.07 mmol) in DMF (5 mL) was added sodium hydride (49.16 mg, 1.28 mmol, 60% dispersion in mineral oil) at 0° C. in portions and the resulting mixture was stirred at RT for 2 h. Upon completion, the mixture was quenched with water (10 mL) was added carefully to quench the reaction, and the mixture was extracted with EA (3×15 mL). The combined organic layers were washed with brine, dried over Na₂SO₄, filtered, and concentrated in vacuo. The residue was purified by flash chromatography on silica gel (eluting with 0-10% MeOH in DCM) to give ethyl 11-ethyl-1,7,9-triazatricyclo[6.3.1.04,12]dodeca-2,4(12), 5,7-tetraene-2-carboxylate (80 mg, 308.52 μmol, 28.8% yield) as a yellow oil. LC/MS (ESI⁺): m/z 259.8 [(M+H)⁺].

### Step 6:

To a mixture of ethyl 11-ethyl-1,7,9-triazatricyclo[6.3.1.04,12]dodeca-2,4(12), 5,7-tetraene-2-carboxylate (80 mg, 308.52 μmol) in anhydrous THF (2 mL) was added LiAlH₄ (15.70 mg, 462.78 μmol) at 0° C. in portions and the resulting mixture was stirred at RT for 1.5 h. Then the reaction was quenched with saturated Na₂SO₄ solution (5 drops). The mixture was filtered and the filtrate was concentrated to give crude (11-ethyl-1,7,9-triazatricyclo[6.3.1.04,12]dodeca-2,4(12), 5,7-tetraen-2-yl)methanol (62 mg, 285.36 μmol, 92.4% yield) as a yellow solid. LC/MS (ESI⁺): m/z 217.9 [(M+H)⁺].

### Step 7:

To a mixture of (11-ethyl-1,7,9-triazatricyclo[6.3.1.04,12]dodeca-2,4(12), 5,7-tetraen-2-yl)methanol (62 mg, 285.36 μmol) in CHCl₃ (3 mL) was added manganese dioxide (76.15 mg, 856.09 μmol) and the resulting mixture was stirred at 66° C. for 3 h. After cooling to RT, the mixture was filtered and the filtrate was concentrated in vacuo to give crude 11-ethyl-1,7,9-triazatricyclo[6.3.1.04,12]dodeca-2,4(12), 5,7-tetraene-2-carbaldehyde (60 mg, 278.74 μmol, 97.6% yield) as a yellow solid. LC/MS (ESI⁺): m/z 215.8 [(M+H)⁺].

### Step 8:

A mixture of 11-ethyl-1,7,9-triazatricyclo[6.3.1.04,12]dodeca-2,4(12), 5,7-tetraene-2-carbaldehyde (35 mg, 162.60 μmol), methyl 3-amino-5-fluoro-4-(methylamino)benzoate (35.45 mg, 178.86 μmol) and disodium hydrosulfite (84.93 mg, 487.80 μmol) in ethanol/H₂O mixed solvents (3 mL, 2:1) was stirred at 80° C. overnight. Then the mixture was diluted with water and extracted with EA (2×5 mL). The combined organic layers were concentrated and dried in vacuo to give crude methyl 2-(11-ethyl-1,7,9-triazatricyclo[6.3.1.04,12]dodeca-2,4(12), 5,7-tetraen-2-yl)-7-fluoro-1-methyl-benzimidazole-5-carboxylate (50 mg, 127.09 μmol, 78.1% yield) as a yellow solid. LC/MS (ESI⁺): m/z 393.8 [(M+H)⁺].

### Step 9:

A mixture of methyl 2-(11-ethyl-1,7,9-triazatricyclo[6.3.1.04,12]dodeca-2,4(12), 5,7-tetraen-2-yl)-7-fluoro-1-methyl-benzimidazole-5-carboxylate (60 mg, 152.51 μmol) and lithium hydroxide monohydrate (32 mg, 762.56 μmol) in THF/MeOH/H₂O mixed solvents (5 mL, 2:2:1) was stirred at RT overnight. Then the mixture was acidified with 4 M HCl in EA (0.5 mL). The mixture was concentrated and purified by flash chromatography on a C18 column [eluting with 20%-40% MeCN in H₂O (with 0.5% formic acid) from 20% to 40%] to give 2-(11-ethyl-1,7,9-triazatricyclo[6.3.1.04,12]dodeca-2,4(12), 5,7-tetraen-2-yl)-7-fluoro-1-methyl-benzimidazole-5-carboxylic acid (34 mg, 89.62 μmol, 58.7% yield) as a yellow solid. LC/MS (ESI⁺): m/z 379.7 [(M+H)⁺].

### Step 10:

A mixture of 2-(11-ethyl-1,7,9-triazatricyclo[6.3.1.04,12]dodeca-2,4(12), 5,7-tetraen-2-yl)-7-fluoro-1-methyl-benzimidazole-5-carboxylic acid (30 mg, 79.07 μmol), HATU (39.09 mg, 102.80 μmol), DIEA (30.66 mg, 237.22 μmol), and tert-butyl (1R,4R,7R)-2-azabicyclo[2.2.1]heptan-7-ylcarbamate (20.14 mg, 94.89 μmol) in DMF (2.0 mL) was stirred at RT for 2 h. Then the mixture was diluted with water (10 mL) and extracted with EA (3×10 mL). The combined organic layers were washed with brine, dried over Na₂SO₄, filtered, and concentrated in vacuo. The residue was purified by flash chromatography on silica gel (eluting with 0-10% MeOH in DCM) to give tert-butyl N-[(1R,4R,7R)-2-[2-(11-ethyl-1,7,9-triazatricyclo[6.3.1.04,12]dodeca-2,4(12), 5,7-tetraen-2-yl)-7-fluoro-1-methyl-benzimidazole-5-carbonyl]-2-azabicyclo[2.2.1]heptan-7-yl]carbamate (13 mg, 22.66 μmol, 28.6% yield) as a yellow solid. LC/MS (ESI⁺): m/z 573.8 [(M+H)⁺].

### Step 11:

To a mixture of tert-butyl N-[(1R,4R,7R)-2-[2-(11-ethyl-1,7,9-triazatricyclo[6.3.1.04,12]dodeca-2,4(12), 5,7-tetraen-2-yl)-7-fluoro-1-methyl-benzimidazole-5-carbonyl]-2-azabicyclo[2.2.1]heptan-7-yl]carbamate (15 mg, 26.15 μmol) in DCM (1 mL) was added TFA (370.00 mg, 3.24 mmol) and the resulting mixture was stirred at RT for 2 h. Then the mixture was concentrated in vacuo and purified by prep-HPLC to give [(1R,4R,7R)-7-amino-2-azabicyclo[2.2.1]heptan-2-yl]-[2-(11-ethyl-1,7,9-triazatricyclo[6.3.1.04,12]dodeca-2,4(12), 5,7-tetraen-2-yl)-7-fluoro-1-methyl-benzimidazol-5-yl]methanone (3.0 mg, 6.34 μmol, 24.2% yield) as a pale yellow sold. LC/MS (ESI⁺): m/z 473.8 [(M+H)⁺]. ¹H NMR (400 MHz, DMSO-d₆) δ 8.24 (s, 1.5H), 7.61-7.51 (m, 1.5H), 7.40 (d, J=6.8 Hz, 1H), 7.15-7.10 (m, 2H), 7.04 (d, J=6.8 Hz, 1H), 4.87-4.85 (m, 1H), 4.54 (s, 3H), 4.08 (t, J=10.0 Hz 1H), 3.76 (s, 1H), 3.69 (dd, J=10.2 Hz, J=6.0 Hz, 2H), 3.20 (s, 1H), 3.08-3.02 (m, 1H), 2.04-1.99 (m, 3H), 1.96-1.85 (m, 2H), 1.78-1.72 (m, 1H), 1.46-1.44 (m, 1H), 0.88 (dd, J=12.4 Hz, J=5.0 Hz, 3H).

### Example 136 Synthesis of ((3R,5R)-3-amino-5-fluoropiperidin-1-yl)(2-(3-ethyl-4,5-dihydro-3H-2a, 5,6-triazaacenaphthylen-2-yl)-7-fluoro-1-methyl-1H-benzo[d]imidazol-5-yl)methanone

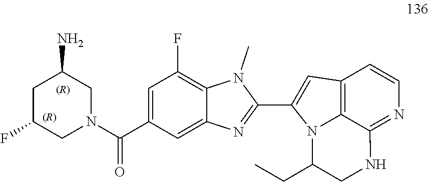

Prepared in analogous manner as for Example 135. LC/MS (ESI⁺): m/z 479.7 [(M+H)⁺].

### Example 137 Preparation of 2-[7-[(1R,4R,7R)-7-amino-2-azabicyclo[2.2.1]heptane-2-carbonyl]-3-methyl-imidazo[1,2-b]pyridazin-2-yl]-11-ethyl-1,9-diazatricyclo[6.3.1.04,12]dodeca-2,4,6,8(12)-tetraen-10-one

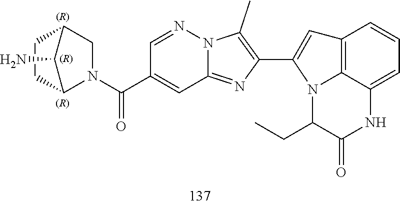

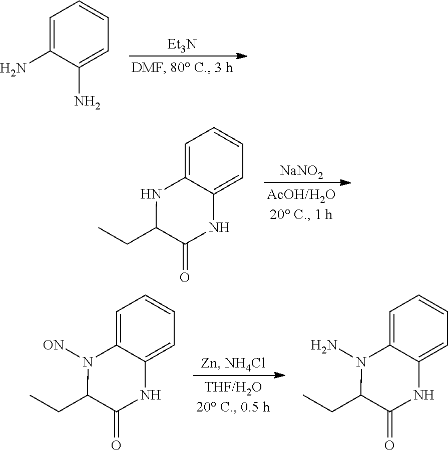

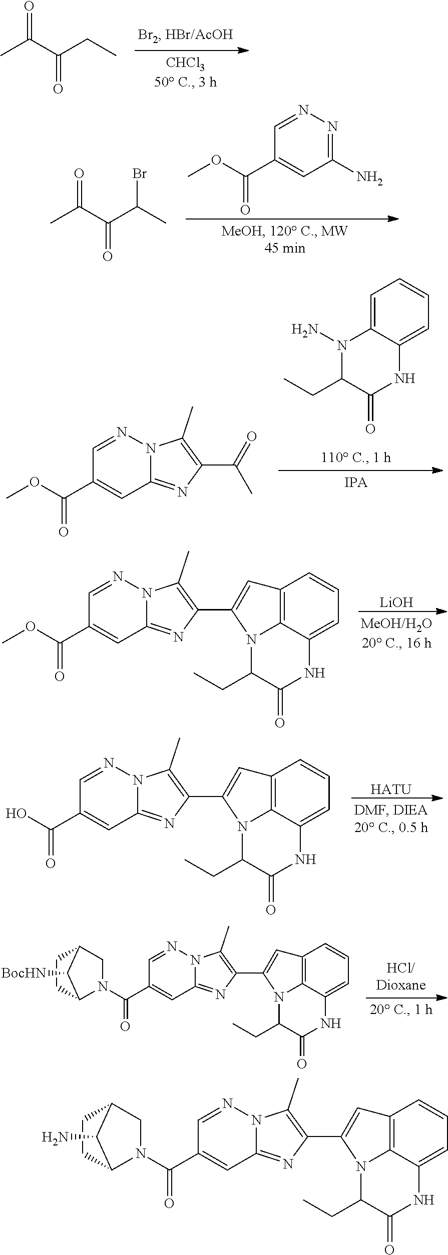

### Step 1:

To a stirred solution of benzene-1,2-diamine (5 g, 46.24 mmol) and triethylamine (9.36 g, 92.47 mmol) in DMF (35 mL) was added ethyl 2-bromobutanoate (10.82 g, 55.48 mmol) dropwise and the resulting mixture was stirred at 50° C. for 2 h. Then the mixture was allowed to warm to 80° C. and stirred for 3 h. After cooling to RT, the mixture concentrated in vacuo. The residue was redissolved in water (100 mL) and extracted with EA (100 mL×2). The combined organic layers were dried over Na₂SO₄, filtered, and concentrated in vacuo. The residue was purified by flash chromatography on silica gel (eluting with 0-50% EA in hexane) to give 3-ethyl-3,4-dihydro-1H-quinoxalin-2-one (3.8 g, 21.56 mmol, 46.6% yield) as a yellow solid. LC/MS (ESI⁺): m/z 176.9 [(M+H)⁺].

### Step 2:

To a stirred solution of 3-ethyl-3,4-dihydro-1H-quinoxalin-2-one (4 g, 22.70 mmol) in AcOH/H₂O mixed solvents (55 mL, 8:3) was added a solution of NaNO₂ (1.64 g, 23.83 mmol) in water (5 mL) dropwise at 20° C. The mixture was stirred at 20° C. for 1 h. White precipitation was collected by filtration and dried in vacuo to afford 3-ethyl-4-nitroso-1,3-dihydroquinoxalin-2-one (3.5 g) as a white solid, which was used in the next step directly without further purification.

### Step 3:

To a stirred solution of 3-ethyl-4-nitroso-1,3-dihydroquinoxalin-2-one (1.5 g, 7.31 mmol) in THF (15 mL) was added a solution of NH₄Cl (2.62 g, 48.97 mmol) in water (15 mL), followed by zinc (1.91 g, 29.24 mmol). Upon completion, the mixture was filtered through celite, diluted with water (50 mL) and extracted with EA (50 mL×2). The combined organic layers were dried over Na₂SO₄, filtered, and concentrated in vacuo to afford 4-amino-3-ethyl-1,3-dihydroquinoxalin-2-one (1.4 g) as an orange solid, which was used in the next step directly without further purification. LC/MS (ESI⁺): m/z 174.9 [(M+H)⁺].

### Step 4:

To a stirred solution of pentane-2,3-dione (5 g, 49.94 mmol) in CHCl₃ (80 mL) was dropwise added a solution of Br₂ (7.98 g, 49.94 mmol) in CHCl₃ (20 mL) and 33 wt % HBr in AcOH (15 drops). Then the mixture was stirred at 50° C. for 3 h before being concentrated in vacuo. The residue was purified by flash column chromatography on silica gel (eluting with 0-6% EA in PE) to give 4-bromopentane-2,3-dione (4 g, 20.11 mmol, 40.2% yield) as a yellow liquid.

### Step 5:

A mixture of methyl 6-aminopyridazine-4-carboxylate (2 g, 13.06 mmol) and 4-bromopentane-2,3-dione (2.81 g, 15.67 mmol) in methanol (40 mL) was stirred at 120° C. with microwave irradiation for 45 min. Then the mixture was concentrated in vacuo, and the residue was purified by flash column chromatography on silica gel (eluting with 0-30% EA in PE) to afford methyl 2-acetyl-3-methyl-imidazo[1,2-b]pyridazine-7-carboxylate (126 mg, 540.26 μmol, 4.1% yield) as a brown solid. LC/MS (ESI⁺): m/z 233.8 [(M+H)⁺].

### Step 6:

A mixture of methyl 2-acetyl-3-methyl-imidazo[1,2-b]pyridazine-7-carboxylate (50 mg, 214.39 μmol) and 4-amino-3-ethyl-1,3-dihydroquinoxalin-2-one (60 mg, 313.76 μmol, HCl salt) in 2-propanol (2.5 mL) was stirred at 110° C. with microwave irradiation for 1 h. After cooling to RT, the mixture was filtered. The filter cake was washed with IPA (5 mL) and dried in vacuo to afford methyl 2-(11-ethyl-10-oxo-1,9-diazatricyclo[6.3.1.0^{4,12}]dodeca-2,4,6,8(12)-tetraen-2-yl)-3-methyl-imidazo[1,2-b]pyridazine-7-carboxylate (32 mg) as a yellow solid, which was used in the next step directly without further purification. LC/MS (ESI⁺): m/z 389.7 [(M+H)⁺].

### Step 7:

A mixture of methyl 2-(11-ethyl-10-oxo-1,9-diazatricyclo[6.3.1.0^{4,12}]dodeca-2,4,6,8(12)-tetraen-2-yl)-3-methyl-imidazo[1,2-b]pyridazine-7-carboxylate (27 mg, 69.34 μmol) and LiOH·H₂O (11.6 mg, 277.35 μmol) in MeOH/THF/H₂O mixed solvents (5 mL, 2:2:1) was stirred at 20° C. for 16 h. Upon completion, the mixture was concentrated in vacuo. The residue was redissolved in water (20 mL), acidized with 1 M HCl and then extracted with EA (20 mL×2). The combined organic layers were dried over Na₂SO₄, filtered and concentrated in vacuo to afford 2-(11-ethyl-10-oxo-1,9-diazatricyclo[6.3.1.0^{4,12}]dodeca-2,4,6,8(12)-tetraen-2-yl)-3-methyl-imidazo[1,2-b]pyridazine-7-carboxylic acid (25 mg) as a yellow solid, which was used in the next step directly without further purification. LC/MS (ESI⁺): m/z 375.7 [(M+H)⁺].

### Step 8:

To a stirred solution of 2-(11-ethyl-10-oxo-1,9-diazatricyclo[6.3.1.0^{4,12}]dodeca-2,4,6,8(12)-tetraen-2-yl)-3-methyl-imidazo[1,2-b]pyridazine-7-carboxylic acid (25 mg, 66.60 μmol), HATU (32.9 mg, 86.58 μmol) and DIEA (25.8 mg, 199.80 μmol) in DMF (3 mL) was added tert-butyl (1R,4R,7R)-2-azabicyclo[2.2.1]heptan-7-ylcarbamate (17 mg, 79.92 μmol). The mixture was stirred at 20° C. for 30 min. Then the mixture was diluted with water (20 mL) and extracted with EA (20 mL×2). The combined organic layers were dried over Na₂SO₄, filtered, and concentrated in vacuo. The residue was purified by prep-TLC (100% EtOAc) to afford tert-butyl N-[(1R,4R,7R)-2-[2-(11-ethyl-10-oxo-1,9-diazatricyclo[6.3.1.0^{4,12}]dodeca-2,4,6,8(12)-tetraen-2-yl)-3-methyl-imidazo[1,2-b]pyridazine-7-carbonyl]-2-azabicyclo[2.2.1]heptan-7-yl]carbamate (27 mg, 47.40 μmol, 71.1% yield) as a yellow oil. LC/MS (ESI⁺): m/z 569.7 [(M+H)⁺].

### Step 9:

To a stirred solution of tert-butyl N-[(1R,4R,7R)-2-[2-(11-ethyl-10-oxo-1,9-diazatricyclo[6.3.1.0^{4,12}]dodeca-2,4,6,8(12)-tetraen-2-yl)-3-methyl-imidazo[1,2-b]pyridazine-7-carbonyl]-2-azabicyclo[2.2.1]heptan-7-yl]carbamate (30 mg, 52.66 μmol) in MeOH (0.5 mL) was added 4 M HCl in dioxane (2.5 mL). The mixture was stirred at 20° C. for 1 h, then concentrated in vacuo and purified by prep-HPLC to afford 2-[7-[(1R,4R,7R)-7-amino-2-azabicyclo[2.2.1]heptane-2-carbonyl]-3-methyl-imidazo[1,2-b]pyridazin-2-yl]-11-ethyl-1,9-diazatricyclo[6.3.1.0^{4,12}]dodeca-2,4,6,8(12)-tetraen-10-one (3.7 mg, 7.89 μmol, 14.9% yield) as a yellow solid. LC/MS (ESI⁺): m/z 469.7 [(M+H)⁺]. ¹H NMR (400 MHz, DMSO-d₆) δ 10.96 (s, 1H), 8.76-8.59 (m, 1H), 8.20 (dd, J=4.2, 1.9 Hz, 1H), 7.21 (dt, J=8.1, 1.0 Hz, 1H), 7.02-6.84 (m, 2H), 6.60 (d, J=7.3 Hz, 1H), 6.07 (dt, J=5.7, 2.7 Hz, 1H), 4.18-3.45 (m, 3H), 3.27-3.06 (m, 2H), 2.79 (d, J=2.7 Hz, 3H), 2.26-2.15 (m, 1H), 2.06-1.57 (m, 5H), 1.42 (td, J=8.2, 4.7 Hz, 1H), 0.40 (tt, J=7.4, 2.7 Hz, 3H).

### Example 138 Preparation of [(1R,4R,7R)-7-amino-2-azabicyclo[2.2.1]heptan-2-yl]-[2-(11-ethyl-1,9-diazatricyclo[6.3.1.0⁴′2]dodeca-2,4,6,8(12)-tetraen-2-yl)-3-methyl-imidazo[1,2-b]pyridazin-7-yl]methanone

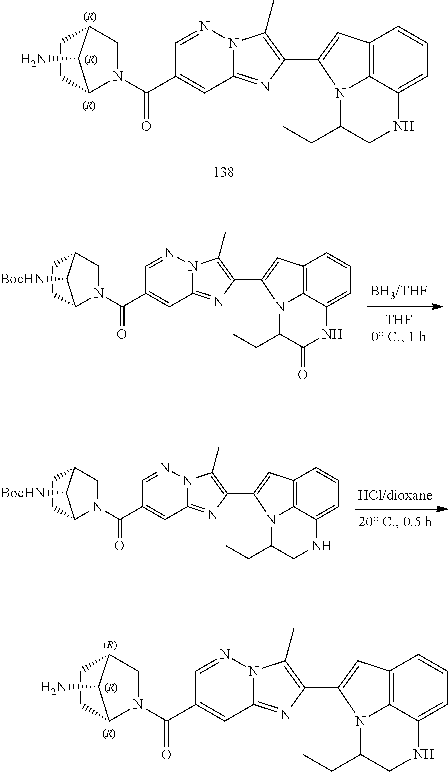

### Step 1:

To a stirred solution of tert-butyl N-[(1R,4R,7R)-2-[2-(11-ethyl-10-oxo-1,9-diazatricyclo[6.3.1.0^{4,12}]dodeca-2,4,6,8(12)-tetraen-2-yl)-3-methyl-imidazo[1,2-b]pyridazine-7-carbonyl]-2-azabicyclo[2.2.1]heptan-7-yl]carbamate (100 mg, 175.55 μmol) in THF (5 mL) was dropwise added 1 M BH₃ in THF (0.7 mL) at 0° C. The mixture was stirred at 0° C. for 1 h, quenched with MeOH (1 mL) and then concentrated in vacuo. The residue was purified by reversed-phase column chromatography to afford tert-butyl N-[(1R,4R,7R)-2-[2-(11-ethyl-1,9-diazatricyclo[6.3.1.0^{4,12}]dodeca-2,4,6,8(12)-tetraen-2-yl)-3-methyl-imidazo[1,2-b]pyridazine-7-carbonyl]-2-azabicyclo[2.2.1]heptan-7-yl]carbamate (12 mg, 20.52 μmol, 11.6% yield) as a yellow solid. LC/MS (ESI⁺): m/z 555.7 [(M+H)⁺].

### Step 2:

To a stirred solution of tert-butyl N-[(1R,4R,7R)-2-[2-(11-ethyl-1,9-diazatricyclo[6.3.1.0^{4,12}]dodeca-2,4,6,8(12)-tetraen-2-yl)-3-methyl-imidazo[1,2-b]pyridazine-7-carbonyl]-2-azabicyclo[2.2.1]heptan-7-yl]carbamate (12 mg, 21.60 μmol) in MeOH (1 mL) was added 4 M HCl in dioxane (2 mL). The mixture was stirred at 20° C. for 0.5 h, and then concentrated in vacuo. The residue was purified by prep-HPLC to afford [(1R,4R,7R)-7-amino-2-azabicyclo[2.2.1]heptan-2-yl]-[2-(11-ethyl-1,9-diazatricyclo[6.3.1.0^{4,12}]dodeca-2,4,6,8(12)-tetraen-2-yl)-3-methyl-imidazo[1,2-b]pyridazin-7-yl]methanone (5.6 mg, 12.29 μmol, 56.9% yield) as a yellow solid. LC/MS (ESI⁺): m/z 455.7 [(M+H)⁺]. ¹H NMR (400 MHz, DMSO-d₆) δ 8.67 (dd, J=17.7, 2.0 Hz, 1H), 8.14 (dd, J=4.3, 2.0 Hz, 1H), 6.89-6.83 (m, 1H), 6.82-6.75 (m, 1H), 6.69 (d, J=2.4 Hz, 1H), 6.29 (d, J=7.1 Hz, 1H), 5.95 (s, 1H), 5.37 (d, J=7.7 Hz, 1H), 3.85-3.70 (m, 1H), 3.58-3.46 (m, 3H), 3.24-3.06 (m, 3H), 2.76 (d, J=3.0 Hz, 3H), 2.26-2.13 (m, 1H), 2.08-1.74 (m, 3H), 1.71-1.53 (m, 2H), 1.43 (dd, J=10.4, 7.2 Hz, 1H), 0.74 (qd, J=5.4, 3.3 Hz, 3H).

### Example 139 Preparation of 2-(5-((1R,4R,7R)-7-amino-2-azabicyclo[2.2.1]heptane-2-carbonyl)-7-fluoro-1-methyl-1H-benzo[d]imidazol-2-yl)-4-ethyl-4,5-dihydro-6H-pyrrolo[3,2,1-ij]quinolin-6-one

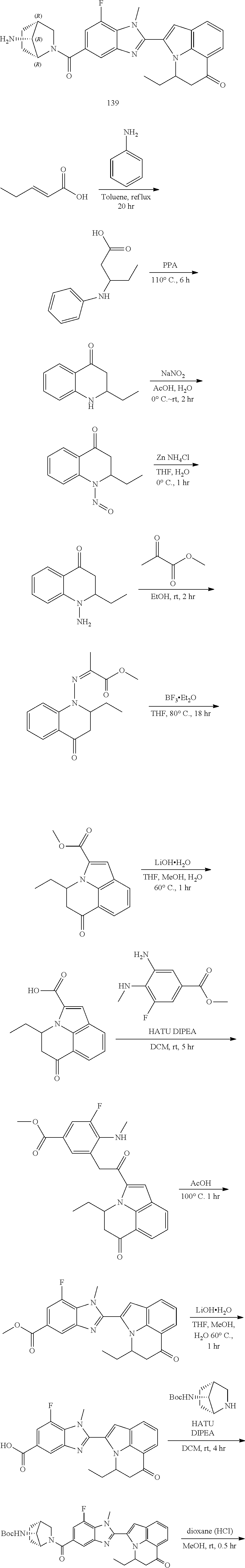

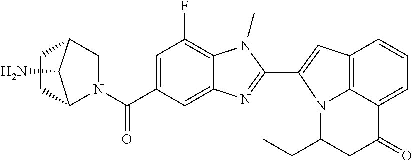 [SMILES: CCC1CC(=O)c2cccc3cc(-c4nc5cc(C(=O)N6C[C@H]7CC[C@@H]6[C@@H]7N)cc(F)c5n4C)n1c23]

### Step 1:

To a solution of (E)-pent-2-enoic acid (25 g, 249.71 mmol) in toluene (200 mL) was added aniline (27.91 g, 299.65 mmol) and the resulting mixture was stirred at 120° C. for 20 h. After cooling to RT, the mixture was concentrated in vacuo. The residue was purified by reverse phase chromatography to obtain 3-anilinopentanoic acid (33.47 g, 173.20 mmol, 69.3% yield) as a grey oil. LC/MS (ESI⁺): m/z 193.8 [(M+H)⁺].

### Step 2:

A mixture of 3-anilinopentanoic acid (33.47 g, 173.20 mmol) in polyphosphoric acid (50 mL) was heated to 110° C. for 6 h. Upon completion, the mixture was poured into ice water (1500 mL), basified with Na₂CO₃ until pH ˜8, and then extracted with EA (3×800 mL). The combined organic layers were washed with water (1000 mL) and brine (1000 mL), dried over sodium sulfate, filtered, and concentrated in vacuo. The residue was purified by flash column chromatography on silica gel (eluting with 0-50% EA in heptane) to obtain 2-ethyl-2,3-dihydro-1H-quinolin-4-one (6.5 g, 37.09 mmol, 21.4% yield) as a white solid. LC/MS (ESI⁺): m/z 175.8 [(M+H)⁺].

### Step 3:

To a mixture of 2-ethyl-2,3-dihydro-1H-quinolin-4-one (2.3 g, 13.13 mmol) in H₂O/AcOH mixed solvents (33 mL, 1:2) was dropwise added a solution of sodium nitrite (950.97 mg, 13.78 mmol) in water (3 mL) at 0° C. A large amount of precipitation was formed, and the mixture was stirred at RT for 2 h. Upon completion, the mixture was filtered, and the filter cake was dried in vacuo to give the 2-ethyl-1-nitroso-2,3-dihydroquinolin-4-one (1.4 g, 6.86 mmol, 52.2% yield) as a faint yellow solid. LC/MS (ESI⁺): m/z 204.7 [(M+H)⁺].

### Step 4:

To a mixture of 2-ethyl-1-nitroso-2,3-dihydroquinolin-4-one (1.4 g, 6.86 mmol) and ammonium chloride (1.83 g, 34.28 mmol) in H₂O/THF mixed solvents (30 mL, 1:1) was added zinc powder (1.79 g, 27.42 mmol) in batches at 0° C. The resulting mixture was stirred at 0° C. for 1 h, then filtered. The filter cake was washed with THF (3×20 mL) and the filtrate was extracted with EA (2×30 mL). The combined organic layers were concentrated in vacuo. The residue was purified by flash column chromatography on silica gel (eluting with 0-80% EA in heptane) to give 1-amino-2-ethyl-2,3-dihydroquinolin-4-one (1.2 g, 6.31 mmol, 92.0% yield) as a white solid. LC/MS (ESI⁺): m/z 190.8 [(M+H)⁺].

### Step 5:

A mixture of 1-amino-2-ethyl-2,3-dihydroquinolin-4-one (1.2 g, 6.31 mmol) and methyl 2-oxopropanoate (676.15 mg, 6.62 mmol) in ethanol (12 mL) was stirred at 50° C. for 3 h. After the reaction was completed, the mixture was concentrated in vacuo to give crude product methyl (2E)-2-[(2-ethyl-4-oxo-2,3-dihydroquinolin-1-yl)imino]propanoate (1.85 g), which was used without further purification. LC/MS (ESI⁺): m/z 274.8 [(M+H)⁺].

### Step 6:

To a stirred solution of methyl (2E)-2-[(2-ethyl-4-oxo-2,3-dihydroquinolin-1-yl)imino]propanoate (1750 mg, 6.38 mmol) in THF (20 mL) was added boron trifluoride etherate (2.26 g, 7.66 mmol) and the resulting mixture was stirred at 80° C. for 18 h. After cooling to RT, the reaction was quenched by addition of saturated aqueous solution of NaHCO₃. The mixture was extracted with ethyl acetate (3×30 mL). The combined organic layers were washed with water (50 mL) and brine (50 mL), dried over sodium sulfate, filtered, and concentrated in vacuo. The residue was purified by flash column chromatography on silica gel (eluting with 0-30% EA in heptane) to obtain methyl 11-ethyl-9-oxo-1-azatricyclo[6.3.1.04,12]dodeca-2,4(12), 5,7-tetraene-2-carboxylate (610 mg, 2.37 mmol, 37.1% yield) as a white solid. LC/MS (ESI⁺): m/z 257.8 [(M+H)⁺].

### Step 7:

To a mixture of methyl 11-ethyl-9-oxo-1-azatricyclo[6.3.1.04,12]dodeca-2,4(12), 5,7-tetraene-2-carboxylate (110 mg, 427.54 μmol) in MeOH/THF mixed solvents (4 mL, 1:3) was added a solution of LiOH·H₂O (53.82 mg, 1.28 mmol) in water (1 mL) and the resulting mixture was stirred at 60° C. for 1 h. After the reaction was completed, the mixture was concentrated in vacuo, diluted with water (3 mL), acidified with 2 M aqueous hydrochloric acid and extracted with CH₂Cl₂ (2×10 mL). The combined organic extracts were washed with brine (20 mL), dried over sodium sulfate and concentrated in vacuo to obtain 11-ethyl-9-oxo-1-azatricyclo[6.3.1.04,12]dodeca-2,4(12), 5,7-tetraene-2-carboxylic acid (100 mg, 411.09 μmol, 96.1% yield). LC/MS (ESI⁺): m/z 243.8 [(M+H)⁺].

### Step 8:

To a solution of 11-ethyl-9-oxo-1-azatricyclo[6.3.1.04,12]dodeca-2,4(12), 5,7-tetraene-2-carboxylic acid (100 mg, 411.09 μmol) and methyl 3-amino-5-fluoro-4-(methylamino)benzoate (85.55 mg, 431.64 μmol) in DMF (5 mL) at RT were added HATU (203.20 mg, 534.41 μmol) and N,N-diisopropylethylamine (159.39 mg, 1.23 mmol). The reaction mixture was stirred at RT for 1 h, then heated to 100° C. for 16 h. Upon completion, the reaction was quenched with H₂O (15 mL) and extracted with CH₂Cl₂ (2×30 mL). The combined organic extracts were washed with brine (20 mL), dried over sodium sulfate, and concentrated in vacuo. The residue was purified by flash column chromatography on silica gel (eluting with 0-80% EA in hexane) to afford methyl 3-[(11-ethyl-9-oxo-1-azatricyclo[6.3.1.04,12]dodeca-2,4(12), 5,7-tetraene-2-carbonyl)amino]-5-fluoro-4-(methylamino)benzoate (100 mg, 236.16 μmol, 57.4% yield) as a bluish white solid. LC/MS (ESI⁺): m/z 423.8 [(M+H)⁺].

### Step 9:

A mixture of methyl 3-[(11-ethyl-9-oxo-1-azatricyclo[6.3.1.04,12]dodeca-2,4(12), 5,7-tetraene-2-carbonyl)amino]-5-fluoro-4-(methylamino)benzoate (100 mg, 236.16 μmol) in acetic acid (6 mL) was stirred at 100° C. for 1.5 h under an atmosphere of N₂. Upon completion, the mixture was concentrated in vacuo, then diluted with EtOAc (20 mL), basified with saturated NaHCO₃, and extracted with EA (2×20 mL). The combined organic extracts were washed with brine (20 mL), dried over sodium sulfate, and evaporated in vacuo. The residue was purified by flash column chromatography on silica gel (eluting with 1-20% MeOH in CH₂Cl₂) to afford methyl 2-(11-ethyl-9-oxo-1-azatricyclo[6.3.1.04,12]dodeca-2,4(12), 5,7-tetraen-2-yl)-7-fluoro-1-methyl-benzimidazole-5-carboxylate (60 mg, 147.99 μmol, 62.6% yield) as a colorless solid. LC/MS (ESI⁺): m/z 405.7 [(M+H)⁺].

### Step 10:

To a solution of methyl 2-(11-ethyl-9-oxo-1-azatricyclo[6.3.1.04,12]dodeca-2,4(12), 5,7-tetraen-2-yl)-7-fluoro-1-methyl-benzimidazole-5-carboxylate (60 mg, 147.99 μmol) in MeOH/THF mixed solvents (4 mL, 1:1) was added a solution of LiOH (18.63 mg, 443.98 μmol) in water (1 mL) and the resulting mixture was stirred at 60° C. for 1 h. After the hydrolysis was completed, the mixture was concentrated in vacuo, diluted with water (3 mL), acidified with 2 M aqueous hydrochloric acid and extracted with CH₂Cl₂ (2×10 mL). The combined organic extracts were washed with brine (20 mL), dried over sodium sulfate, filtered, and concentrated in vacuo to obtain 2-(11-ethyl-9-oxo-1-azatricyclo[6.3.1.04,12]dodeca-2,4(12), 5,7-tetraen-2-yl)-7-fluoro-1-methyl-benzimidazole-5-carboxylic acid (50 mg, 127.75 μmol, 86.3% yield). LC/MS (ESI⁺): m/z 391.8 [(M+H)⁺].

### Step 11:

To a stirred solution of 2-(11-ethyl-9-oxo-1-azatricyclo[6.3.1.04,12]dodeca-2,4(12), 5,7-tetraen-2-yl)-7-fluoro-1-methyl-benzimidazole-5-carboxylic acid (50 mg, 127.75 μmol) and tert-butyl N-[(1R,4R,7R)-2-azabicyclo[2.2.1]heptan-7-yl]carbamate (27.12 mg, 127.75 μmol) in CH₂Cl₂ (4.0 mL) at RT were added HATU (63.15 mg, 166.07 μmol) and N,N-diisopropylethylamine (49.53 mg, 383.25 μmol). The reaction mixture was stirred at RT for 4 h. After the reaction was completed, the mixture was concentrated in vacuo. The residue was purified by flash column chromatography on silica gel (eluting with 1-20% MeOH in CH₂Cl₂) to afford tert-butyl N-[(1R,4R,7R)-2-[2-(11-ethyl-9-oxo-1-azatricyclo[6.3.1.04,12]dodeca-2,4(12), 5,7-tetraen-2-yl)-7-fluoro-1-methyl-benzimidazole-5-carbonyl]-2-azabicyclo[2.2.1]heptan-7-yl]carbamate (65 mg, 110.98 μmol, 86.8% yield) as a colorless solid. LC/MS (ESI⁺): m/z 585.8 [(M+H)⁺].

### Step 12:

To a stirred solution tert-butyl N-[(1R,4R,7R)-2-[2-(11-ethyl-9-oxo-1-azatricyclo[6.3.1.04,12]dodeca-2,4(12), 5,7-tetraen-2-yl)-7-fluoro-1-methyl-benzimidazole-5-carbonyl]-2-azabicyclo[2.2.1]heptan-7-yl]carbamate (65 mg, 110.98 μmol) in MeOH (1 mL) was added 4 M HCl in dioxane (3 mL) and the resulting mixture was stirred at RT for 0.5 h. Then the reaction mixture was concentrated in vacuo. The residue was purified by prep-HPLC to give 2-[5-[(1R,4R,7R)-7-amino-2-azabicyclo[2.2.1]heptane-2-carbonyl]-7-fluoro-1-methyl-benzimidazol-2-yl]-11-ethyl-1-azatricyclo[6.3.1.04,12]dodeca-2,4(12), 5,7-tetraen-9-one (35 mg, 72.08 mole, 64.9% yield) as a white solid. LC/MS (ESI⁺): m/z 485.8 [(M+H)⁺]. ¹H NMR (400 MHz, DMSO-d₆) δ 8.18 (s, 0.42H), 8.04 (d, J=7.8 Hz, 1H), 7.73 (d, J=37.6 Hz, 1H), 7.65 (d, J=5.4 Hz, 1H), 7.35 (d, J=5.4 Hz, 1H), 7.32 (dd, J=10.1, 6.8 Hz, 1H), 7.30-7.22 (m, 1H), 5.62 (p, J=5.8 Hz, 1H), 4.20 (s, 3H), 3.91 (d, J=13.8 Hz, 1H), 3.73-3.66 (m, 1H), 3.63 (d, J=11.4 Hz, 1H), 3.33 (s, 1H), 3.12 (d, J=10.8 Hz, 1H), 2.91 (d, J=16.2 Hz, 1H), 2.40 (s, 1H), 2.03-1.87 (m, 2H), 1.85-1.75 (m, 1H), 1.61-1.53 (m, 2H), 1.52-1.46 (m, 1H), 0.58-0.39 (m, 3H).

### Example 140 Preparation of [(1R,4R,7R)-7-amino-2-azabicyclo[2.2.1]heptan-2-yl]-[2-(11-ethyl-1,9-diazatricyclo[6.3.1.0^{4,12}]dodeca-2,4,6,8(12)-tetraen-2-yl)-4-fluoro-3-methyl-pyrazolo[1,5-a]pyridin-6-yl]methanone

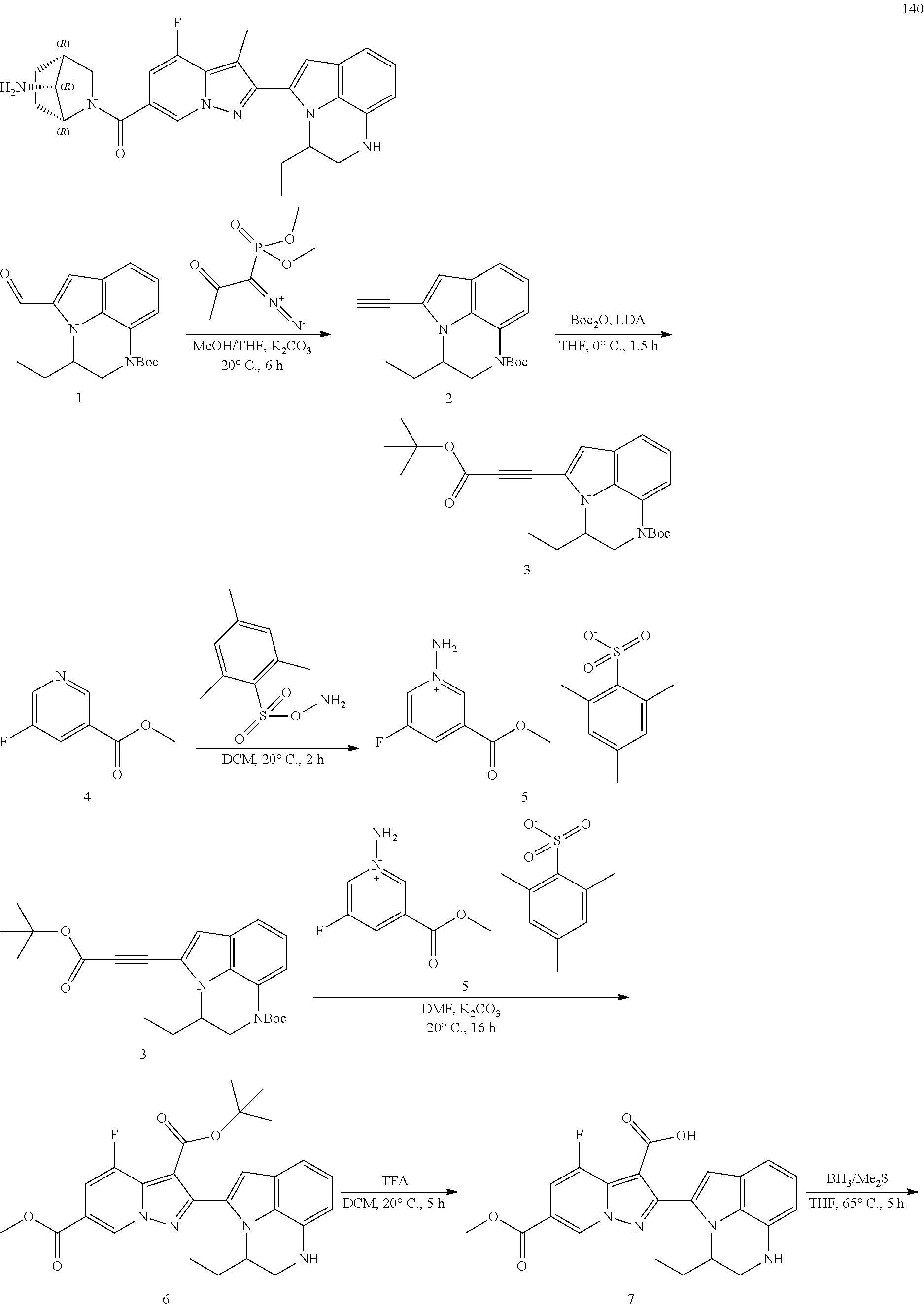

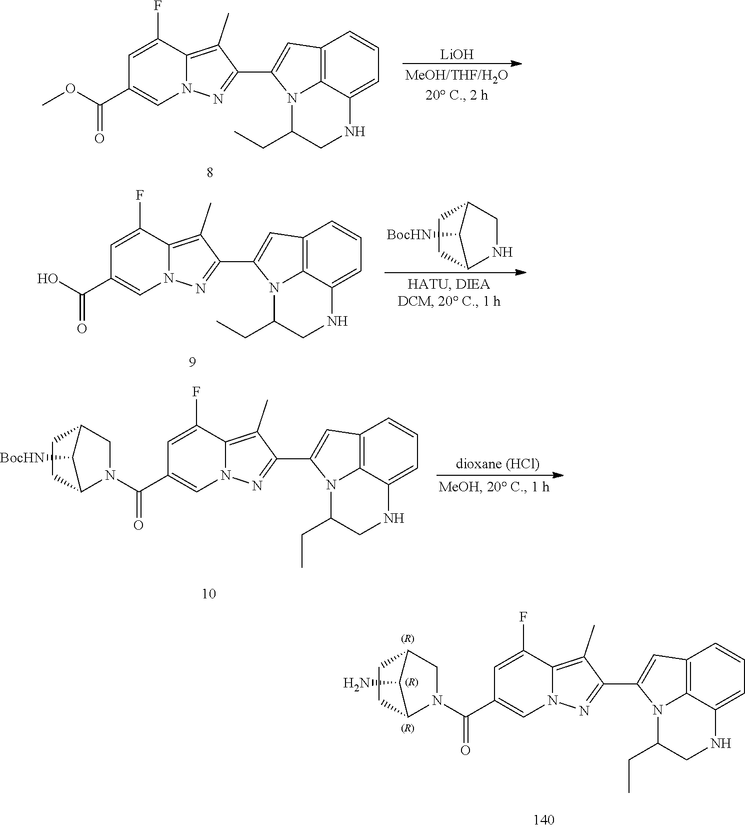

### Step 1:

A mixture of 1-diazonio-1-dimethoxyphosphorylprop-1-en-2-olate (1.19 g, 6.20 mmol) and K₂CO₃ (1.98 g, 14.31 mmol) in MeOH (20 mL) was stirred at 0° C. for 0.5 h. Then a solution of tert-butyl 11-ethyl-2-formyl-1,9-diazatricyclo[6.3.1.0^{4,12}]dodeca-2,4,6,8(12)-tetraene-9-carboxylate (1.5 g, 4.77 mmol) in MeOH (5 mL) was dropwise added into above mixture. The mixture was stirred at 20° C. for 6 h. Then the mixture was diluted with water (100 mL) and extracted with EA (50 mL×2). The combined organic layers were dried over Na₂SO₄, filtered, and concentrated in vacuo. The residue was purified by flash column chromatography on silica gel (eluting with 0-20% EA in PE) to afford tert-butyl 11-ethyl-2-ethynyl-1,9-diazatricyclo[6.3.1.0^{4,12}]dodeca-2,4,6,8(12)-tetraene-9-carboxylate (1.25 g, 4.03 mmol, 84.4% yield) as a colorless oil. LC/MS (ESI⁺): m/z 310.8 [(M+H)⁺].

### Step 2:

To a stirred solution of tert-butyl 11-ethyl-2-ethynyl-1,9-diazatricyclo[6.3.1.0^{4,12}]dodeca-2,4,6,8(12)-tetraene-9-carboxylate (1.25 g, 4.03 mmol) in THF (20 mL) was added (diisopropylamino)lithium (647.11 mg, 6.04 mmol) dropwise at 0° C. The mixture was stirred at 0° C. for 0.5 h before a solution of Boc₂O (1.14 g, 5.24 mmol, 1.20 mL) in THF (5 mL) was added dropwise. Stirring was continued for 1 h. After the reaction was completed, water (80 mL) was added and the mixture was extracted with EA (60 mL×2). The combined organic layers were dried over Na₂SO₄, filtered, and concentrated in vacuo. The residue was purified by flash column chromatography on silica gel (eluting with 0-20% EA in PE) to afford tert-butyl 2-(3-tert-butoxy-3-oxo-prop-1-ynyl)-11-ethyl-1,9-diazatricyclo[6.3.1.0^{4,12}]dodeca-2,4,6,8(12)-tetraene-9-carboxylate (1.27 g, 3.10 mmol, 77.0% yield) as a white solid. LC/MS (ESI⁺): m/z 410.8 [(M+H)⁺].

### Step 3:

To a stirred solution of methyl 5-fluoropyridine-3-carboxylate (2 g, 12.89 mmol) in DCM (40 mL) was added amino 2,4,6-trimethylbenzenesulfonate (2.78 g, 12.89 mmol) at 0° C. The resulting mixture was stirred at 20° C. for 2 h. The mixture was filtered and the filter cake was dried in vacuo to afford 1-amino-3-fluoro-5-(methoxycarbonyl)pyridin-1-ium 2,4,6-trimethylbenzenesulfonate (1.3 g) as a white solid, which was used in the next step directly without further purification.

### Step 4:

To a stirred mixture of tert-butyl 2-(3-tert-butoxy-3-oxo-prop-1-ynyl)-11-ethyl-1,9-diazatricyclo[6.3.1.0^{4,12}]dodeca-2,4,6,8(12)-tetraene-9-carboxylate (680 mg, 1.66 mmol) in DMF (14 mL) were added 1-amino-3-fluoro-5-(methoxycarbonyl)pyridin-1-ium 2,4,6-trimethylbenzenesulfonate (613.6 mg, 1.66 mmol) and K₂CO₃ (457.9 mg, 3.31 mmol). The resulting mixture was stirred at 20° C. for 16 h. After the reaction was completed, water (70 mL) was added and the mixture was extracted with EA (60 mL×2). The combined organic layers were dried over Na₂SO₄, filtered, and concentrated in vacuo. The residue was purified by flash column chromatography on silica gel (eluting with 030% EA in PE) to afford 03-tert-butyl 06-methyl 2-(9-tert-butoxycarbonyl-11-ethyl-1,9-diazatricyclo[6.3.1.0^{4,12}]dodeca-2,4,6,8(12)-tetraen-2-yl)-4-fluoro-pyrazolo[1,5-a]pyridine-3,6-dicarboxylate (303 mg, 525.38 μmol, 31.7% yield) as a yellow oil. LC/MS (ESI⁺): m/z 578.7 [(M+H)⁺].

### Step 5:

To a stirred solution of 03-tert-butyl 06-methyl 2-(9-tert-butoxycarbonyl-11-ethyl-1,9-diazatricyclo[6.3.1.0^{4,12}]dodeca-2,4,6,8(12)-tetraen-2-yl)-4-fluoro-pyrazolo[1,5-a]pyridine-3,6-dicarboxylate (320 mg, 553.03 μmol) in DCM (6 mL) was added TFA (4 mL), and the resulting mixture was stirred at 20° C. for 5 h. After the reaction was completed, the mixture was concentrated in vacuo, diluted with water (30 mL), basified with saturated NaHCO₃ aqueous to pH=10, and extracted with EA (20 mL). Then the aqueous layer was acidized with 1 M HCl to PH=5, and the mixture was extracted with EA (30 mL×2). The combined organic layers were dried over Na₂SO₄, filtered and concentrated in vacuo to afford 2-(11-ethyl-1,9-diazatricyclo[6.3.1.0^{4,12}]dodeca-2,4,6,8(12)-tetraen-2-yl)-4-fluoro-6-methoxycarbonyl-pyrazolo[1,5-a]pyridine-3-carboxylic acid (140 mg) as a yellow oil, which was used in the next step directly without further purification. LC/MS (ESI⁺): m/z 422.7 [(M+H)⁺].

### Step 6:

To a stirred solution of 2-(11-ethyl-1,9-diazatricyclo[6.3.1.0^{4,12}]dodeca-2,4,6,8(12)-tetraen-2-yl)-4-fluoro-6-methoxycarbonyl-pyrazolo[1,5-a]pyridine-3-carboxylic acid (140 mg, 331.43 μmol) in THF (5 mL) was added BH₃·SMe₂ (125.9 mg, 1.66 mmol) dropwise at 0° C., and the resulting mixture was stirred at 0° C. for 0.5 h, then at 65° C. for 5 h. After the reaction was completed, MeOH was added to quench the reaction and the mixture was concentrated in vacuo. The residue was purified by prep-TLC to afford methyl 2-(11-ethyl-1,9-diazatricyclo[6.3.1.0^{4,12}]dodeca-2,4,6,8(12)-tetraen-2-yl)-4-fluoro-3-methyl-pyrazolo[1,5-a]pyridine-6-carboxylate (18 mg, 44.85 μmol, 13.5% yield) as a colorless oil. LC/MS (ESI⁺): m/z 392.7 [(M+H)⁺].

### Step 7:

To a stirred mixture of methyl 2-(11-ethyl-1,9-diazatricyclo[6.3.1.0^{4,12}]dodeca-2,4,6,8(12)-tetraen-2-yl)-4-fluoro-3-methyl-pyrazolo[1,5-a]pyridine-6-carboxylate (27 mg, 68.80 μmol) in MeOH/THF/H₂O mixed solvents (2.1 mL, 3:3:1) was added LiOH·H₂O (8.7 mg, 206.41 μmol). The resulting mixture was stirred at 20° C. for 2 h. After the reaction was completed, the mixture was concentrated in vacuo. The residue was diluted with water (20 mL), acidified with HCl (1 M), and extracted with EA (20 mL×2). The combined organic layers were dried over Na₂SO₄, filtered and concentrated in vacuo to afford 2-(11-ethyl-1,9-diazatricyclo[6.3.1.0^{4,12}]dodeca-2,4,6,8(12)-tetraen-2-yl)-4-fluoro-3-methyl-pyrazolo[1,5-a]pyridine-6-carboxylic acid (25 mg) as a colorless oil, which was used in the next step directly without further purification. LC/MS (ESI⁺): m/z 378.7 [(M+H)⁺].

### Step 8:

To a stirred solution of 2-(11-ethyl-1,9-diazatricyclo[6.3.1.0^{4,12}]dodeca-2,4,6,8(12)-tetraen-2-yl)-4-fluoro-3-methyl-pyrazolo[1,5-a]pyridine-6-carboxylic acid (25 mg, 66.07 μmol) in DMF (3 mL) was successively added HATU (32.7 mg, 85.89 μmol), DIEA (25.6 mg, 198.20 μmol) and tert-butyl N-[(1R,4R,7R)-2-azabicyclo[2.2.1]heptan-7-yl]carbamate (16.8 mg, 79.28 μmol). The resulting mixture was stirred at 20° C. for 1 h. After the reaction was completed, water (30 mL) was added and the mixture was extracted with EA (30 mL×2). The combined organic layers were dried over Na₂SO₄, filtered, and concentrated in vacuo. The residue was purified by prep-TLC to afford tert-butyl ((1R,4R,7R)-2-(2-(3-ethyl-2,3-dihydro-1H-pyrrolo[1,2,3-de]quinoxalin-5-yl)-4-fluoro-3-methylpyrazolo[1,5-a]pyridine-6-carbonyl)-2-azabicyclo[2.2.1]heptan-7-yl)carbamate (30 mg, 52.39 μmol, 79.2% yield) as a yellow oil. LC/MS (ESI⁺): m/z 572.7 [(M+H)⁺].

### Step 9:

To a stirred solution of tert-butyl N-[(1R,4R,7R)-2-[2-(11-ethyl-1,9-diazatricyclo[6.3.1.0^{4,12}]dodeca-2,4,6,8(12)-tetraen-2-yl)-4-fluoro-3-methyl-pyrazolo[1,5-a]pyridine-6-carbonyl]-2-azabicyclo[2.2.1]heptan-7-yl]carbamate (30 mg, 52.39 μmol) in MeOH (1 mL) was added 4 M HCl in dioxane (2 mL). The mixture was stirred at 20° C. for 1 h before solvent was removed in vacuo. The residue was purified by prep-HPLC to afford [(1R,4R,7R)-7-amino-2-azabicyclo[2.2.1]heptan-2-yl]-[2-(11-ethyl-1,9-diazatricyclo[6.3.1.0^{4,12}]dodeca-2,4,6,8(12)-tetraen-2-yl)-4-fluoro-3-methyl-pyrazolo[1,5-a]pyridin-6-yl]methanone (10.4 mg, 22.01 μmol, 42.0% yield) as a white solid. LC/MS (ESI⁺): m/z 472.7 [(M+H)⁺]. ¹H NMR (400 MHz, CD₃OD) δ 8.72-8.57 (m, 1H), 7.12-6.95 (m, 2H), 6.91-6.83 (m, 1H), 6.76 (d, J=1.5 Hz, 1H), 6.41 (dd, J=7.3, 0.8 Hz, 1H), 5.15 (ddt, J=8.2, 5.2, 2.4 Hz, 1H), 4.67-4.40 (m, 1H), 4.12 (s, 1H), 3.73-3.47 (m, 3H), 3.25 (d, J=11.4 Hz, 1H), 2.62 (s, 3H), 2.48 (s, 1H), 2.14-1.93 (m, 3H), 1.78-1.59 (m, 3H), 0.82 (q, J=7.3 Hz, 3H).

### Example 141 Preparation of 7-(5-((1R,4R,7R)-7-amino-2-azabicyclo[2.2.1]heptane-2-carbonyl)-7-fluoro-1-methyl-1H-benzo[d]imidazol-2-yl)-8-(cyclopropylmethyl)-3,3-dimethyl-3,8-dihydropyrrolo[3,2-g]indol-2(1H)-one

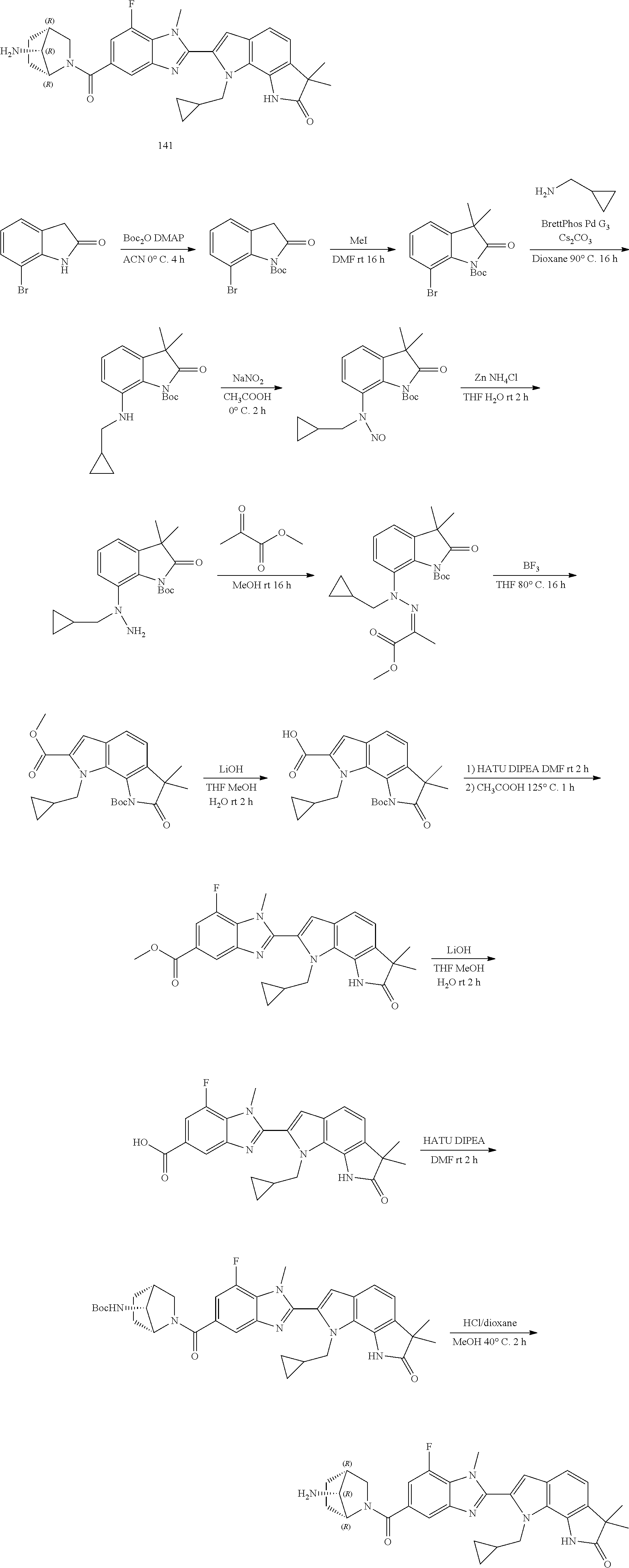

### Step 1:

A mixture of 7-bromoindolin-2-one (5.0 g, 23.58 mmol), DMAP (4.32 g, 35.37 mmol) and di-tert-butyl dicarbonate (6.18 g, 28.30 mmol) in anhydrous MeCN (50 mL) was stirred at 0° C. for 4 h. The reaction was monitored by LC/MS until full conversion of the starting material. Upon completion, the reaction mixture was carefully poured into water (30 mL), extracted with EA, washed with brine, and dried over anhydrous sodium sulfate. After filtration and evaporation of the solvent in vacuo, the residue was purified by flash column chromatography on silica gel (eluting with 0-10% EA in PE) to give tert-butyl 7-bromo-2-oxoindoline-1-carboxylate (7.2 g, 23.07 mmol, 97.8% yield) as a yellow solid. LC/MS (ESI⁺): m/z 255.6. [(M+H-56)⁺]

### Step 2:

To a stirred solution of tert-butyl 7-bromo-2-oxo-indoline-1-carboxylate (7.0 g, 22.42 mmol) in anhydrous DMF (70 mL) at 0° C. was added NaH (1.61 g, 67.27 mmol) in portions. The resulting mixture was stirred for 30 min before iodomethane (9.55 g, 67.27 mmol, 4.19 mL) was added dropwise. Stirring was continued for 16 h at RT. The mixture was carefully quenched with water (30 mL), extracted with EA, washed with brine, and dried over anhydrous sodium sulfate. After filtration and evaporation of the solvent in vacuo, the residue was purified by flash column chromatography on silica gel (eluting with 0-10% EA in PE) to give tert-butyl 7-bromo-3,3-dimethyl-2-oxo-indoline-1-carboxylate (4.6 g, 13.52 mmol, 60.3% yield) as a yellow solid. LC/MS (ESI⁺): m/z 285.6 [(M+H-56)⁺].

### Step 3:

A mixture of tert-butyl 7-bromo-3,3-dimethyl-2-oxo-indoline-1-carboxylate (4.6 g, 13.52 mmol), cesium carbonate (13.22 g, 40.56 mmol), cyclopropylmethanamine (1.92 g, 27.04 mmol), and BrettPhos-Pd-G3 (1.23 g, 1.35 mmol) in dioxane (50 mL) under N₂ atmosphere was stirred at 90° C. for 16 h. After cooling to RT, the mixture was diluted with EA, washed with brine, and dried over anhydrous Na₂SO₄. After filtration and removal of the solvent in vacuo, the crude mixture was purified by silica gel column chromatography (eluting with 0-10% EA in PE) to give tert-butyl 7-((cyclopropylmethyl)amino)-3,3-dimethyl-2-oxoindoline-1-carboxylate (3.5 g, 10.59 mmol, 78.3% yield) as a yellow solid. LC/MS (ESI⁺): m/z 330.8 [(M+H)⁺].

### Step 4:

To a stirred solution of tert-butyl 7-(cyclopropylmethylamino)-3,3-dimethyl-2-oxo-indoline-1-carboxylate (3.5 g, 10.59 mmol) in CH₃COOH (30 mL) was added sodium nitrite (730.89 mg, 10.59 mmol) in H₂O (10 mL) at 0° C. The resulting mixture was stirred for 2 h at 0° C. and monitored by the LC/MS until the reaction was completed. The mixture was diluted with DCM and washed with brine, dried over Na₂SO₄, filtered, and concentrated in vacuo to give tert-butyl 7-((cyclopropylmethyl)(nitroso)amino)-3,3-dimethyl-2-oxoindoline-1-carboxylate (3.6 g, 10.02 mmol, 94.5% yield) as a yellow solid. LC/MS (ESI⁺): m/z 329.9 [(M+H-30)⁺].

### Step 5:

To a stirred solution of tert-butyl 7-[cyclopropylmethyl(nitroso)amino]-3,3-dimethyl-2-oxo-indoline-1-carboxylate (3.6 g, 10.02 mmol) in THF (40 mL) were added ammonium chloride (2.14 g, 40.06 mmol) in H₂O (40 mL) and zinc powder (2.62 g, 40.06 mmol). The resulting mixture was stirred vigorously at RT for 2 h and monitored by LC/MS until the reaction was completed. The mixture was filtered and the filtrate was extracted with EA (30 mL×2), dried over Na₂SO₄, filtered and concentrated in vacuo to afford tert-butyl 7-(1-(cyclopropylmethyl)hydrazineyl)-3,3-dimethyl-2-oxoindoline-1-carboxylate (3.2 g) as a yellow solid which was used in the next step directly without further purification. LC/MS (ESI⁺): m/z 345.8 [(M+H)⁺].

### Step 6:

To a stirred solution of tert-butyl 7-[amino(cyclopropylmethyl)amino]-3,3-dimethyl-2-oxo-indoline-1-carboxylate (3.2 g, 9.26 mmol) in EtOH (30 mL) was added methyl 2-oxopropanoate (945.71 mg, 9.26 mmol) under N₂ atmosphere. The resulting mixture was stirred at RT for 16 h and concentrated in vacuo. The residue was slurried in a mixed solvent of DCM (16.0 mL) and PE (80 mL), filtered and the filter cake was dried in vacuum to afford tert-butyl (Z)-7-(1-(cyclopropylmethyl)-2-(1-methoxy-1-oxopropan-2-ylidene)hydrazineyl)-3,3-dimethyl-2-oxoindoline-1-carboxylate (3.2 g, 7.45 mmol, 80.4% yield) as a yellow solid. LC/MS (ESI⁺): m/z 328.8 [(M+H-100)⁺].

### Step 7:

To a stirred solution of tert-butyl 7-[cyclopropylmethyl-[(Z)-(2-methoxy-1-methyl-2-oxo-ethylidene)amino]amino]-3,3-dimethyl-2-oxo-indoline-1-carboxylate (3.2 g, 7.45 mmol) in THF (30 mL) was added boron trifluoride diethyl etherate (3.17 g, 22.35 mmol) under N₂ atmosphere. The resulting mixture was heated to 80° C. and stirred for 16 h. After cooling to RT, the mixture was concentrated in vacuo and the residue was diluted with DCM, washed with brine, and dried over anhydrous Na₂SO₄. After filtration and evaporation of the solvent, the crude mixture was purified by flash column chromatography on silica gel(eluting with 0-5% MeOH in DCM) to afford 01-tert-butyl 07-ethyl 8-(cyclopropylmethyl)-3,3-dimethyl-2-oxo-pyrrolo[3,2-g]indole-1,7-dicarboxylate (180 mg, 422.04 μmol, 5.6% yield) as a yellow solid. LC/MS (ESI⁺): m/z 356.8 [(M+H-56)⁺].

### Step 8:

To a stirred solution of 01-tert-butyl 07-methyl 8-(cyclopropylmethyl)-3,3-dimethyl-2-oxo-pyrrolo[3,2-g]indole-1,7-dicarboxylate (180 mg, 436.39 μmol) in THF/MeOH (3 mL, 2:1) mixed solvents was added LiOH aqueous solution(1.0 M, 1.75 mL). The mixture was stirred at RT for 2 h, then acidified to pH=5-6 with 3 M hydrochloric acid aqueous solution and extracted with DCM (20 mL×3). The organic phase was dried over anhydrous sodium sulfate and filtered. The filtrate was concentrated in vacuo to give 1-tert-butoxycarbonyl-8-(cyclopropylmethyl)-3,3-dimethyl-2-oxo-pyrrolo[3,2-g]indole-7-carboxylic acid (150 mg, 376.46 μmol, 86.2% yield) as a white solid. LC/MS (ESI⁺): m/z 343.1 [(M+H-56)⁺].

### Step 9:

A solution of 1-tert-butoxycarbonyl-8-(cyclopropylmethyl)-3,3-dimethyl-2-oxo-pyrrolo[3,2-g]indole-7-carboxylic acid (150 mg, 376.46 μmol), DIPEA (146.0 mg, 1.13 mmol), HATU (171.8 mg, 451.75 μmol) and methyl 3-amino-5-fluoro-4-(methylamino)benzoate (78.4 mg, 395.28 μmol) in DMF (2 mL) was stirred at RT for 2 h and monitored by LC/MS. The mixture was diluted with EA, washed with brine, and dried over anhydrous sodium sulfate. After filtration and evaporation of the solvent in vacuo, the residue was redissolved in CH₃COOH (2 mL) and stirred at 125° C. for 1 h. After cooling to RT and removal of solvent in vacuo, the mixture was diluted with EA (80 mL), washed with saturated Na₂CO₃ solution, dried over Na₂SO₄, filtered, and concentrated in vacuo. The residue was purified by flash column chromatography on silica gel (eluting with 0-10% MeOH in DCM) to give tert-butyl 8-(cyclopropylmethyl)-7-(7-fluoro-5-methoxycarbonyl-1-methyl-benzimidazol-2-yl)-3,3-dimethyl-2-oxo-pyrrolo[3,2-g]indole-1-carboxylate (30 mg, 53.51 μmol, 14.2% yield) as a yellow solid. LC/MS (ESI⁺): m/z 561.3 [(M+H)⁺].

### Step 10:

To a stirred solution of tert-butyl 8-(cyclopropylmethyl)-7-(7-fluoro-5-methoxycarbonyl-1-methyl-benzimidazol-2-yl)-3,3-dimethyl-2-oxo-pyrrolo[3,2-g]indole-1-carboxylate (18 mg, 32.11 μmol) in THF/MeOH mixed solvents (3 mL, 2:1) was added LiOH aqueous solution (1.0 M, 0.20 mL). The mixture was stirred at RT for 2 h, then acidified to pH=5-6 with 3 M hydrochloric acid aqueous solution and extracted with DCM (20 mL×3). The organic phase was dried over anhydrous sodium sulfate, filtered, and concentrated in vacuo to give 2-[1-tert-butoxycarbonyl-8-(cyclopropylmethyl)-3,3-dimethyl-2-oxo-pyrrolo[3,2-g]indol-7-yl]-7-fluoro-1-methyl-benzimidazole-5-carboxylic acid (17 mg, 31.10 μmol, 96.8% yield) as a white solid. LC/MS (ESI⁺): m/z 547.3 [(M+H)⁺].

### Step 11:

a mixture of 2-[1-tert-butoxycarbonyl-8-(cyclopropylmethyl)-3,3-dimethyl-2-oxo-pyrrolo[3,2-g]indol-7-yl]-7-fluoro-1-methyl-benzimidazole-5-carboxylic acid (17 mg, 31.10 μmol), DIPEA (12.1 mg, 93.31 μmol), HATU (17.7 mg, 46.65 μmol) and tert-butyl ((1R,4R,7R)-2-azabicyclo[2.2.1]heptan-7-yl)carbamate (7.9 mg, 37.32 μmol) in DMF (2 mL) was stirred at RT for 2 h and monitored by LC/MS. Upon completion, the mixture was diluted with EA, washed with brine, dried over anhydrous sodium sulfate, filtered, and concentrated in vacuo. The residue was purified by flash column chromatography on silica gel (eluting with 0-8% MeOH in DCM) to give tert-butyl 7-[5-[(1R,4R,7R)-7-(tert-butoxycarbonylamino)-2-azabicyclo[2.2.1]heptane-2-carbonyl]-7-fluoro-1-methyl-benzimidazol-2-yl]-8-(cyclopropylmethyl)-3,3-dimethyl-2-oxo-pyrrolo[3,2-g]indole-1-carboxylate (12 mg, 16.20 μmol, 52.1% yield) as a white solid. LC/MS (ESI⁺): m/z 741.4 [(M+H)⁺].

### Step 12:

To a stirred solution of tert-butyl 7-[5-[(1R,4R,7R)-7-(tert-butoxycarbonylamino)-2-azabicyclo[2.2.1]heptane-2-carbonyl]-7-fluoro-1-methyl-benzimidazol-2-yl]-8-(cyclopropylmethyl)-3,3-dimethyl-2-oxo-pyrrolo[3,2-g]indole-1-carboxylate (12 mg, 16.20 μmol) in MeOH (2 mL) was added 4 M HCl in dioxane (2 mL). The mixture was stirred at 40° C. for 2 h, then concentrated in vacuo, and basified to pH=8 with saturated Na₂CO₃ solution. The mixture was extracted with DCM (30 mL×3), dried over anhydrous sodium sulfate, filtered, and concentrated in vacuo. The residue was purified by flash column chromatography on silica gel (eluting with 0-10% MeOH in DCM) to give 7-[5-[(1R,4R,7R)-7-amino-2-azabicyclo[2.2.1]heptane-2-carbonyl]-7-fluoro-1-methyl-benzimidazol-2-yl]-8-(cyclopropylmethyl)-3,3-dimethyl-1H-pyrrolo[3,2-g]indol-2-one (5 mg, 9.25 μmol, 57.1% yield) as a white solid. LC/MS (ESI⁺): m/z 541.3 [(M+H)⁺].

### Example 142 Preparation of ((1R,4R,7R)-7-amino-2-azabicyclo[2.2.1]heptan-2-yl)(2-(1-(cyclopropylmethyl)-6,6-dimethyl-1,6,7,8-tetrahydropyrrolo[3,2-g]indol-2-yl)-7-fluoro-1-methyl-1H-benzo[d]imidazol-5-yl)methanone

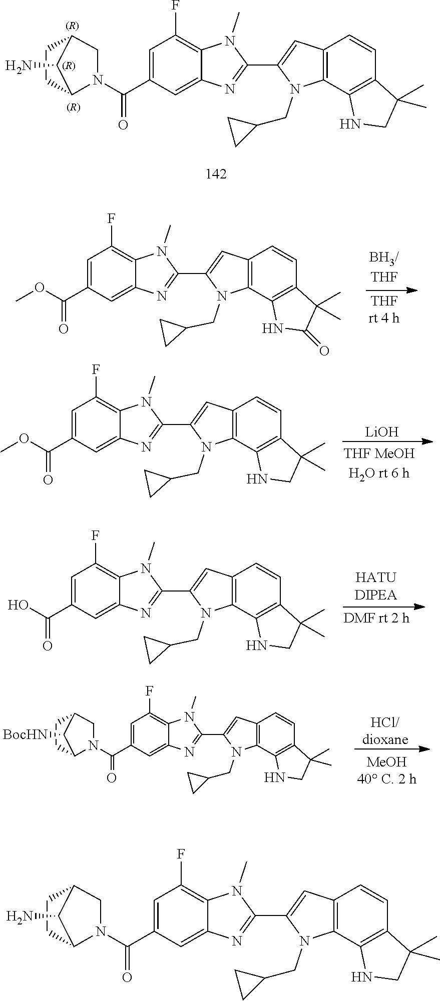

### Step 1:

To a stirred solution of methyl 2-[8-(cyclopropylmethyl)-3,3-dimethyl-2-oxo-1H-pyrrolo[3,2-g]indol-7-yl]-7-fluoro-1-methyl-benzimidazole-5-carboxylate (9.8 mg, 21.28 μmol, intermediate of example 141) in anhydrous THF (2 mL) was dropwise added borane tetrahydrofuran (1 M, 0.1 mL) at 0° C. The resulting mixture was stirred at RT for 4 h, then quenched with MeOH at 0° C. and concentrated in vacuo. The residue was diluted with 2 M HCl aqueous solution (2 mL) and stirred at RT for 1 h. 4 M NaOH aqueous solution was used to basify the solution to pH=8.

The resulting mixture was extracted with DCM (30 mL×3), dried over anhydrous sodium sulfate, filtered, and concentrated in vacuo. The residue was purified by flash column chromatography on silica gel (eluting with 0-5% MeOH in DCM) to give methyl 2-[1-(cyclopropylmethyl)-6,6-dimethyl-7,8-dihydropyrrolo[3,2-g]indol-2-yl]-7-fluoro-1-methyl-benzimidazole-5-carboxylate (6 mg, 13.44 μmol, 63.1% yield) as a yellow solid. LC/MS (ESI⁺): m/z 447.2 [(M+H)⁺].

### Step 2:

To a stirred solution of methyl 2-[1-(cyclopropylmethyl)-6,6-dimethyl-7,8-dihydropyrrolo[3,2-g]indol-2-yl]-7-fluoro-1-methyl-benzimidazole-5-carboxylate (6 mg, 13.44 μmol) in THF/MeOH (3 mL, 2:1) mixed solvents was added LiOH aqueous solution (1.0 M, 0.6 mL). The resulting mixture was stirred at RT for 2 h, then acidified to pH=5-6 with 3 M hydrochloric acid aqueous solution and extracted with DCM (20 mL×3). The organic phase was dried over anhydrous sodium sulfate, filtered, and concentrated in vacuo to give 2-[1-(cyclopropylmethyl)-6,6-dimethyl-7,8-dihydropyrrolo[3,2-g]indol-2-yl]-7-fluoro-1-methyl-benzimidazole-5-carboxylic acid (5.5 mg, 12.72 μmol, 94.6% yield) as a white solid. LC/MS (ESI⁺): m/z 433.2 [(M+H)⁺].

### Step 3:

A solution 2-[1-(cyclopropylmethyl)-6,6-dimethyl-7,8-dihydropyrrolo[3,2-g]indol-2-yl]-7-fluoro-1-methyl-benzimidazole-5-carboxylic acid (5.5 mg, 12.72 μmol), DIPEA (4.9 mg, 38.15 μmol), HATU (7.3 mg, 19.08 μmol), tert-butyl ((1R,4R,7R)-2-azabicyclo[2.2.1]heptan-7-yl)carbamate (3.3 mg, 15.26 μmol) in DMF (2 mL) was stirred at RT for 2 h and monitored by LC/MS. Upon completion, the mixture was diluted with EA, washed with brine, dried over anhydrous sodium sulfate, filtered, and removal of solvent in vacuo. The residue was purified by flash column chromatography on silica gel (eluting with 0-8% MeOH in DCM) to give tert-butyl ((1R,4R,7R)-2-(2-(1-(cyclopropylmethyl)-6,6-dimethyl-1,6,7,8-tetrahydropyrrolo[3,2-g]indol-2-yl)-7-fluoro-1-methyl-1H-benzo[d]imidazole-5-carbonyl)-2-azabicyclo[2.2.1]heptan-7-yl)carbamate (5 mg, 7.98 μmol, 62.7% yield) as a white solid. LC/MS (ESI⁺): m/z 627.4 [(M+H)⁺].

### Step 4:

To a stirred solution of tert-butyl N-[(1R,4R,7R)-2-[2-[1-(cyclopropylmethyl)-6,6-dimethyl-7,8-dihydropyrrolo[3,2-g]indol-2-yl]-7-fluoro-1-methyl-benzimidazole-5-carbonyl]-2-azabicyclo[2.2.1]heptan-7-yl]carbamate (5.0 mg, 7.98 μmol) in MeOH (2 mL) was added 4 M HCl in dioxane (2 mL). The resulting mixture was stirred at 40° C. for 2 h, then concentrated in vacuo and basified to pH=8 with saturated Na₂CO₃ solution. The mixture was extracted with DCM (30 mL×3), dried over anhydrous sodium sulfate, filtered, and concentrated in vacuo. The residue was purified by flash column chromatography on silica gel (eluting with 0-10% MeOH in DCM) to give [(1R,4R,7R)-7-amino-2-azabicyclo[2.2.1]heptan-2-yl]-[2-[1-(cyclopropylmethyl)-6,6-dimethyl-7,8-dihydropyrrolo[3,2-g]indol-2-yl]-7-fluoro-1-methyl-benzimidazol-5-yl]methanone (2 mg, 3.80 μmol, 47.6% yield) as a white solid. LC/MS (ESI⁺): m/z 527.3 [(M+H)⁺].

### Example 143 Preparation of ((1R,4R,7R)-7-amino-2-azabicyclo[2.2.1]heptan-2-yl)(2-(1-(cyclopropylmethyl)-1,6,7,8-tetrahydropyrrolo[2,3-e]indol-2-yl)-7-fluoro-1-methyl-1H-benzo[d]imidazol-5-yl)methanone

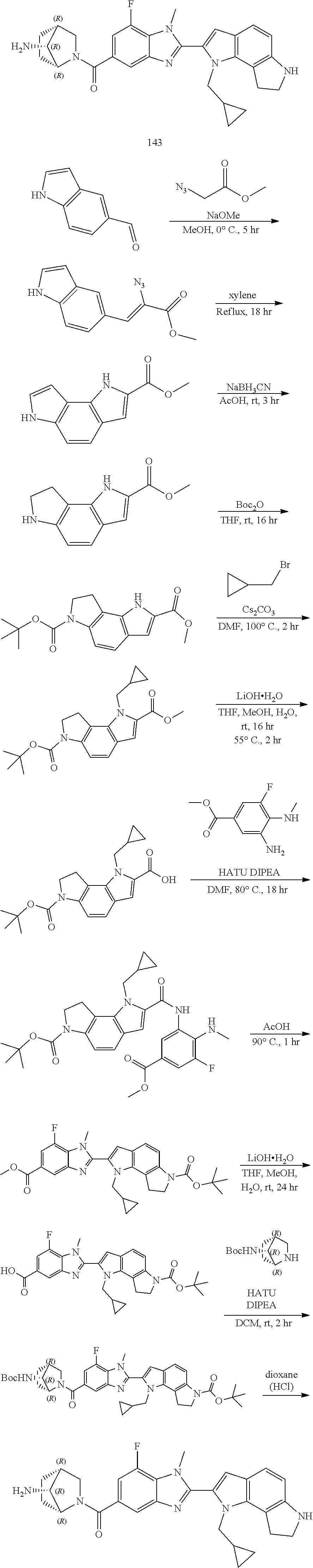

### Step 1:

To a cooled solution of sodium methoxide (1.64 g, 30.31 mmol) in methanol (15 mL) at −10° C. were added a solution of 1H-indole-5-carbaldehyde (1.1 g, 7.58 mmol) and methyl azido acetate (3.49 g, 30.31 mmol, 2.95 mL) in MeOH (10 mL) dropwise over 0.5 h under N₂ atmosphere. The resulting reaction mixture was stirred at 0° C. for 5 h. Then the heterogeneous mixture was diluted with 50 mL of water and extracted with CHCl₃ (50 mL×3). The combined organic layers were washed with water, and concentrated in vacuo to afford methyl (Z)-2-azido-3-(1H-indol-5-yl)prop-2-enoate (1.25 g, crude) as a yellow solid. LC/MS (ESI⁺): m/z 242.7 [(M+H)⁺].

### Step 2:

A suspension of methyl (Z)-2-azido-3-(1H-indol-5-yl)prop-2-enoate (1.25 g, 5.16 mmol) in anhydrous xylenes (15 mL) was refluxed under N₂ atmosphere for 18 h. Then solvent was removed in vacuo and the residue was purified by reversed-phase column chromatography to obtain methyl 1,6-dihydropyrrolo[2,3-e]indole-2-carboxylate (0.8 g, 3.73 mmol, 72.3% yield) as a light red solid. LC/MS (ESI⁺): m/z 214.8 [(M+H)⁺].

### Step 3:

To a stirred solution of methyl 1,6-dihydropyrrolo[2,3-e]indole-2-carboxylate (0.8 g, 3.73 mmol) in acetic acid (12 mL) was added sodium cyanoborohydride (704.03 mg, 11.20 mmol) in batches at 10° C.-15° C. The resulting suspension was stirred at this temperature for 3 h. The reaction mixture was poured into ice water (60 mL), basified with Na₂CO₃ to pH ˜8 and extracted with EA (3×50 mL). The combined organic layers were washed with water (50 mL) and brine (50 mL), dried over sodium sulfate and concentrated in vacuo to obtain methyl 1,6,7,8-tetrahydropyrrolo[2,3-e]indole-2-carboxylate (0.8 g, 3.70 mmol, 99.0% yield) as a white solid. LC/MS (ESI⁺): m/z 216.8 [(M+H)⁺].

### Step 4:

To a stirred solution of methyl 1,6,7,8-tetrahydropyrrolo[2,3-e]indole-2-carboxylate (0.8 g, 3.70 mmol) in THF (10 mL) was added Boc₂O (888.20 mg, 4.07 mmol) at RT. The resulting mixture was stirred at RT for 16 h. Solvent was removed in vacuo and the residue was purified by flash column chromatography on silica gel (eluting with 0-50% EA in PE) to give 6-(tert-butyl) 2-methyl 7,8-dihydropyrrolo[2,3-e]indole-2,6(1H)-dicarboxylate (1.15 g, 3.64 mmol, 98.2% yield) as a white solid. LC/MS (ESI⁺): m/z 316.8 [(M+H)⁺].

### Step 5:

To a suspension of 6-(tert-butyl) 2-methyl 7,8-dihydropyrrolo[2,3-e]indole-2,6(1H)-dicarboxylate (1.15 g, 3.64 mmol) and cesium carbonate (1.78 g, 5.45 mmol) in DMF (15 mL) was added (bromomethyl)cyclopropane (588.90 mg, 4.36 mmol). The resulting mixture was stirred at 100° C. for 2 h under N₂ atmosphere and monitored by LC/MS. Upon completion, the mixture was cooled down to RT, quenched with H₂O (50 mL) and extracted with EA (2×50 mL). Combined organic extracts were washed with brine (20 mL), dried over sodium sulfate and evaporated in vacuo. The residue was purified by flash column chromatography on silica gel (eluting 0-50% EA in PE) to give 6-(tert-butyl) 2-methyl 1-(cyclopropylmethyl)-7,8-dihydropyrrolo[2,3-e]indole-2,6(1H)-dicarboxylate (1.3 g, 3.51 mmol, 96.5% yield) as a white solid. LC/MS (ESI⁺): m/z 370.8 [(M+H)⁺].

### Step 6:

To a stirred solution of 6-(tert-butyl) 2-methyl 1-(cyclopropylmethyl)-7,8-dihydropyrrolo[2,3-e]indole-2,6(1H)-dicarboxylate (1.3 g, 3.51 mmol) in MeOH/THF mixed solvents (10 mL, 2:3) was added a solution of LiOH (736.26 mg, 17.55 mmol) in water (4 mL) and the resulting mixture was stirred at RT for 16 h, then heated to 55° C. for 2 h. Upon completion, the mixture was concentrated in vacuo, taken up in water (6 mL), acidified with 2 M aqueous hydrochloric acid and extracted with CH₂Cl₂ (2×20 mL). Combined organic extracts were washed with brine (20 mL), dried over sodium sulfate and concentrated in vacuo to obtain 6-tert-butoxycarbonyl-1-(cyclopropylmethyl)-7,8-dihydropyrrolo[2,3-e]indole-2-carboxylic acid (1.3 g, 3.65 mmol, 103.9% yield). LC/MS (ESI⁺): m/z 356.8 [(M+H)⁺].

### Step 7:

To a stirred solution of 6-tert-butoxycarbonyl-1-(cyclopropylmethyl)-7,8-dihydropyrrolo[2,3-e]indole-2-carboxylic acid (1.3 g, 3.65 mmol) and methyl 3-amino-5-fluoro-4-(methylamino)benzoate (722.90 mg, 3.65 mmol) in DMF (15 mL) at RT were added HATU (1.80 g, 4.74 mmol) and DIPEA (1.41 g, 10.94 mmol, 1.91 mL). The reaction mixture was stirred at RT for 1 h, then heated to 80° C. for 18 h. Upon completion, the reaction was quenched with H₂O (50 mL) and extracted with CH₂Cl₂(2×50 mL). Combined organic extracts were washed with brine (50 mL), dried over sodium sulfate, and evaporated to give crude product. The residue was purified by flash column chromatography on silica gel (eluting with 2-80% EtOAc in PE) to afford title product tert-butyl 1-(cyclopropylmethyl)-2-[[3-fluoro-5-methoxycarbonyl-2-(methylamino)phenyl]carbamoyl]-7,8-dihydropyrrolo[2,3-e]indole-6-carboxylate (1.0 g, 1.86 mmol, 51.0% yield) as a white solid. LC/MS (ESI⁺): m/z 536.7 [(M+H)⁺].

### Step 8:

A mixture of tert-butyl 1-(cyclopropylmethyl)-2-[[3-fluoro-5-methoxycarbonyl-2-(methylamino)phenyl]carbamoyl]-7,8-dihydropyrrolo[2,3-e]indole-6-carboxylate (1.0 g, 1.86 mmol) in acetic acid (8 mL) was stirred at 90° C. for 1.5 h under an atmosphere of N₂. Upon completion, the reaction mixture was concentrated in vacuo, diluted with EtOAc (20 mL), basified with NaHCO₃ (sat.) (15 mL), and extracted with EtOAc (2×20 mL). Combined organic extracts were washed with brine (20 mL), dried over sodium sulfate, and evaporated to give the crude product. The residue was purified by flash column chromatography on silica gel (eluting with 1-20% MeOH in CH₂Cl₂) to afford title product tert-butyl 1-(cyclopropylmethyl)-2-(7-fluoro-5-methoxycarbonyl-1-methyl-benzimidazol-2-yl)-7,8-dihydropyrrolo[2,3-e]indole-6-carboxylate (900 mg, 1.74 mmol, 93.1% yield) as a colorless solid. LC/MS (ESI⁺): m/z 518.7 [(M+H)⁺].

### Step 9:

To a stirred solution of tert-butyl 1-(cyclopropylmethyl)-2-(7-fluoro-5-methoxycarbonyl-1-methyl-benzimidazol-2-yl)-7,8-dihydropyrrolo[2,3-e]indole-6-carboxylate (0.15 g, 289.25 μmol) in THF/MeOH (5 mL, 3:2) mixed solvents was added a solution of LiOH (60.69 mg, 1.45 mmol) in water (1 mL) and the resulting mixture was stirred at RT for 24 h. The mixture was concentrated in vacuo, taken up in water (5 mL), acidified with 2 M aqueous hydrochloric acid and extracted with MeOH/CH₂Cl₂ (2×20 mL). Combined organic layers were dried over anhydrous sodium sulphate, filtered, and concentrated in vacuo to afford the product 2-[6-tert-butoxycarbonyl-1-(cyclopropylmethyl)-7,8-dihydropyrrolo[2,3-e]indol-2-yl]-7-fluoro-1-methyl-benzimidazole-5-carboxylic acid (140 mg, 277.47 μmol, 95.9% yield) as an off white solid. LC/MS (ESI⁺): m/z 504.8 [(M+H)⁺].

### Step 10:

To a stirred solution of 2-[6-tert-butoxycarbonyl-1-(cyclopropylmethyl)-7,8-dihydropyrrolo[2,3-e]indol-2-yl]-7-fluoro-1-methyl-benzimidazole-5-carboxylic acid (140 mg, 277.47 μmol) and tert-butyl N-[(1R,4R,7R)-2-azabicyclo[2.2.1]heptan-7-yl]carbamate (58.90 mg, 277.47 μmol) in CH₂Cl₂ (5.0 mL) at RT were added HATU (137.16 mg, 360.72 μmol) and DIPEA (107.58 mg, 832.42 μmol). The resulting mixture was stirred at RT for 2 h. Upon completion, solvent was removed in vacuo. The residue was purified by flash column chromatography on silica gel (eluting with 0-20% MeOH in CH₂Cl₂) to afford tert-butyl 2-[5-[(1R,4R,7R)-7-(tert-butoxycarbonylamino)-2-azabicyclo[2.2.1]heptane-2-carbonyl]-7-fluoro-1-methyl-benzimidazol-2-yl]-1-(cyclopropylmethyl)-7,8-dihydropyrrolo[2,3-e]indole-6-carboxylate (170 mg, 243.27 μmol, 87.6% yield) as a colorless solid. LC/MS (ESI⁺): m/z 698.6 [(M+H)⁺].

### Step 11:

To a stirred solution tert-butyl 2-[5-[(1R,4R,7R)-7-(tert-butoxycarbonylamino)-2-azabicyclo[2.2.1]heptane-2-carbonyl]-7-fluoro-1-methyl-benzimidazol-2-yl]-1-(cyclopropylmethyl)-7,8-dihydropyrrolo[2,3-e]indole-6-carboxylate (170 mg, 243.27 μmol) in MeOH (1 mL) was added 4 M HCl in dioxane (3 mL) and the resulting mixture was stirred at RT for 0.5 h. Solent was removed and the residue was purified by prep-HPLC to give HCl salt of [(1R,4R,7R)-7-amino-2-azabicyclo[2.2.1]heptan-2-yl]-[2-[1-(cyclopropylmethyl)-7,8-dihydro-6H-pyrrolo[2,3-e]indol-2-yl]-7-fluoro-1-methyl-benzimidazol-5-yl]methanone (106 mg, 198.11 μmol, 81.4% yield) as a white solid. LC/MS (ESI⁺): m/z 498.7 [(M+H)⁺]. ¹H NMR (400 MHz, DMSO-d₆) δ 9.11 (s, 0.4H), 8.17 (s, 1H), 8.00-7.68 (m, 1H), 7.64-7.54 (m, 1H), 7.43-7.30 (m, 1H), 7.29-7.18 (m, 1H), 6.94 (s, 0.5H), 6.57 (d, J=8.4 Hz, 0.5H), 4.49 (dd, J=11.8, 6.7 Hz, 1H), 4.07 (s, 2H), 3.67 (d, J=11.0 Hz, 1H), 3.61 (d, J=8.4 Hz, 1H), 3.54 (d, J=8.2 Hz, 1H), 3.39 (dd, J=19.8, 11.8 Hz, 1H), 3.28 (d, J=10.4 Hz, 1H), 3.10 (d, J=10.6 Hz, 1H), 2.34 (s, 1H), 1.94 (d, J=7.8 Hz, 1H), 1.79 (t, J=9.2 Hz, 1H), 1.48 (d, J=9.8 Hz, 1H), 1.01-0.87 (m, 1H), 0.24 (t, J=7.8 Hz, 2H), −0.20 (dd, J=9.0, 4.6 Hz, 1H).

### Example 144 Preparation of ((1R,4R,7R)-7-amino-2-azabicyclo[2.2.1]heptan-2-yl)(2-(1-(cyclopropylmethyl)-1,6-dihydropyrrolo[2,3-e]indol-2-yl)-7-fluoro-1-methyl-1H-benzo[d]imidazol-5-yl)methanone

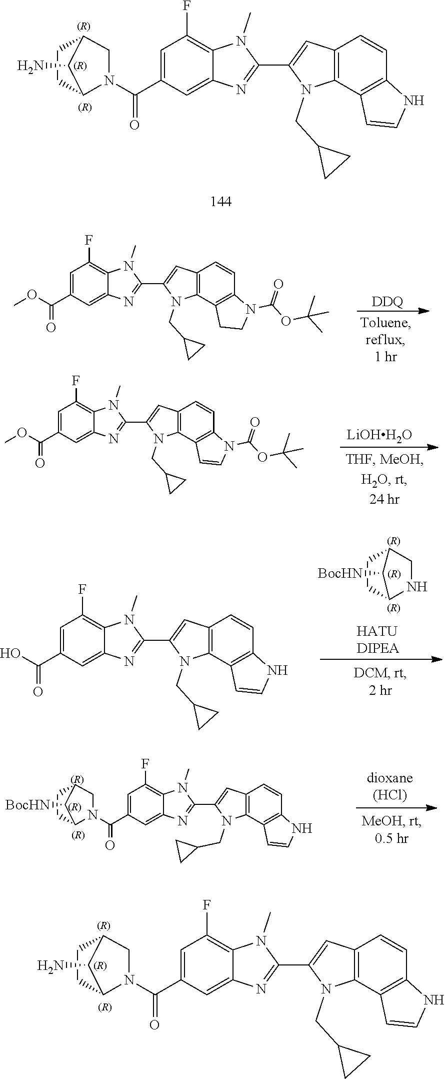

### Step 1:

A solution of tert-butyl 1-(cyclopropylmethyl)-2-(7-fluoro-5-methoxycarbonyl-1-methyl-benzimidazol-2-yl)-7,8-dihydropyrrolo[2,3-e]indole-6-carboxylate (115 mg, 221.76 μmol, intermediate of example 143) and DDQ (60.41 mg, 266.11 μmol) in toluene (5 mL) was stirred at 120° C. for 1 h. The reaction mixture was cooled down to RT, poured into water (15 mL) and extracted with EA (3×15 mL). The combined organic layers were washed with water (20 mL) and brine (20 mL), dried over sodium sulfate, and concentrated in vacuo. The residue was purified by flash column chromatography on silica gel (eluting with 0-20% EA in PE) to obtain tert-butyl 1-(cyclopropylmethyl)-2-(7-fluoro-5-methoxycarbonyl-1-methyl-benzimidazol-2-yl)pyrrolo[2,3-e]indole-6-carboxylate (85 mg, 164.55 μmol, 74.2% yield) as a white solid. LC/MS (ESI⁺): m/z 516.7 [(M+H)⁺].

### Step 2:

To a stirred solution of tert-butyl 1-(cyclopropylmethyl)-2-(7-fluoro-5-methoxycarbonyl-1-methyl-benzimidazol-2-yl)pyrrolo[2,3-e]indole-6-carboxylate (85 mg, 164.55 μmol) in a mixed solent of MeOH (4 mL) and THF (6 mL) was added a solution of LiOH (34.52 mg, 822.75 μmol) in water (2 mL) and the resulting mixture was stirred at RT for 24 h. Upon completion, the reaction mixture was concentrated in vacuo, taken up in water (6 mL), acidified with 2 M aqueous hydrochloric acid and extracted with CH₂Cl₂ (2×20 mL). Combined organic extracts were washed with brine (20 mL), dried over sodium sulfate and evaporated to obtain 62-[1-(cyclopropylmethyl)-6H-pyrrolo[2,3-e]indol-2-yl]-7-fluoro-1-methyl-benzimidazole-5-carboxylic acid (65 mg, 161.52 μmol, 98.1% yield). LC/MS (ESI⁺): m/z 402.8 [(M+H)⁺].

### Step 3:

To a stirred solution of 2-[1-(cyclopropylmethyl)-6H-pyrrolo[2,3-e]indol-2-yl]-7-fluoro-1-methyl-benzimidazole-5-carboxylic acid (65 mg, 161.52 μmol) and tert-butyl N-[(1R,4R,7R)-2-azabicyclo[2.2.1]heptan-7-yl]carbamate (34.29 mg, 161.52 μmol) in CH₂Cl₂ (5.0 mL) at RT were added HATU (79.84 mg, 209.98 μmol) and DIPEA (62.63 mg, 484.57 μmol). The resulting mixture was stirred at RT for 2 h. After the reaction was completed, solvent was removed in vacuo and the residue was purified by flash column chromatography on silica gel (eluting with 0-20% MeOH in CH₂Cl₂) to afford tert-butyl N-[(1R,4R,7R)-2-[2-[1-(cyclopropylmethyl)-6H-pyrrolo[2,3-e]indol-2-yl]-7-fluoro-1-methyl-benzimidazole-5-carbonyl]-2-azabicyclo[2.2.1]heptan-7-yl]carbamate (60 mg, 100.55 μmol, 62.2% yield) as a colorless solid. LC/MS (ESI⁺): m/z 596.7 [(M+H)⁺].

### Step 4:

To a stirred solution tert-butyl N-[(1R,4R,7R)-2-[2-[1-(cyclopropylmethyl)-6H-pyrrolo[2,3-e]indol-2-yl]-7-fluoro-1-methyl-benzimidazole-5-carbonyl]-2-azabicyclo[2.2.1]heptan-7-yl]carbamate (60 mg, 100.55 μmol) in MeOH (0.5 mL) was added 4 M HCl in dioxane (3 mL) and the resulting mixture was stirred at RT for 0.5 h. Then the mixture was concentrated in vacuo and purified by prep-HPLC to give [(1R,4R,7R)-7-amino-2-azabicyclo[2.2.1]heptan-2-yl]-[2-[1-(cyclopropylmethyl)-6H-pyrrolo[2,3-e]indol-2-yl]-7-fluoro-1-methyl-benzimidazol-5-yl]methanone (30 mg, 60.41 μmol, 60.0% yield) as a white solid. LC/MS (ESI⁺): m/z 496.7 [(M+H)⁺]. ¹H NMR (400 MHz, DMSO-d₆) δ 11.43 (s, 1H), 8.25 (s, 0.5H), 7.65 (d, J=45.1 Hz, 1H), 7.38 (dd, J=5.8, 3.0 Hz, 1H), 7.28 (t, J=6.3 Hz, 1H), 7.20 (d, J=11.4 Hz, 1H), 7.11 (s, 1H), 6.86 (s, 1H), 4.77 (d, J=6.8 Hz, 2H), 4.10 (s, 3H), 3.52 (s, 1H), 3.36 (s, 1H), 3.22 (s, 1H), 3.08 (d, J=11.0 Hz, 1H), 2.21 (d, J=24.7 Hz, 1H), 1.94 (d, J=7.6 Hz, 2H), 1.73 (dd, J=17.2, 9.8 Hz, 1H), 1.43 (dd, J=18.0, 8.8 Hz, 1H), 1.21-1.09 (m, 1H), 0.27 (d, J=8.0 Hz, 2H), 0.06 (t, J=5.0 Hz, 2H).

### Example 145 Synthesis of 2-(5-((1R,4R,7R)-7-amino-2-azabicyclo[2.2.1]heptane-2-carbonyl)-7-fluoro-1-methyl-1H-benzo[d]imidazol-2-yl)-1-(cyclopropylmethyl)-6,8-dihydropyrrolo[2,3-e]indol-7(1H)-one

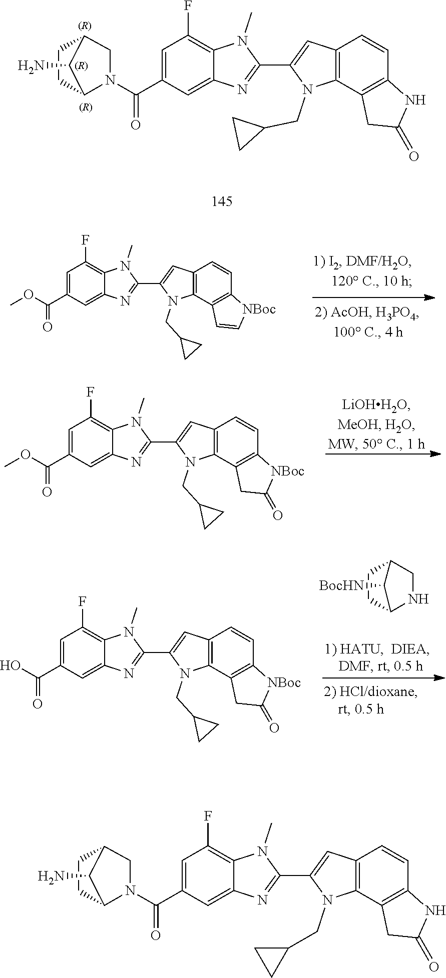

### Step 1:

To a stirred solution of tert-butyl 1-(cyclopropylmethyl)-2-(7-fluoro-5-(methoxycarbonyl)-1-methyl-1H-benzo[d]imidazol-2-yl)pyrrolo[2,3-e]indole-6(1H)-carboxylate (260 mg, 0.50 mmol, intermediate of example 144) in DMF/H₂O (6 mL, 1:2) mixed solvents was added I₂ (127 mg, 0.50 mmol). The resulting mixture was stirred at 120° C. and monitored by LC/MS until the reaction was completed. The reaction was cooled to RT, quenched by aqueous Na₂S₂O₄, diluted with EA, washed with water and brine, dried over Na₂SO₄, and concentrated in vacuo. The residue was redissolved in AcOH (3 mL) and 85% H₃PO₄ (1.2 mL) was added. The resulting mixture was stirred at 100° C. for 4 h. After the reaction was cooled down to RT, the solution was basified with 10 M NaOH to pH=6 and extracted with EA (3×40 mL). The combined organic layers were washed with saturated brine (20 mL), dried over anhydrous sodium sulfate, filtered and concentrated to afford tert-butyl 1-(cyclopropylmethyl)-2-(7-fluoro-5-(methoxycarbonyl)-1-methyl-1H-benzo[d]imidazol-2-yl)-7-oxo-7,8-dihydropyrrolo[2,3-e]indole-6(1H)-carboxylate (50 mg) as a yellow oil, which was used without further purification. LC/MS (ESI⁺): m/z 533.2 [(M+H)⁺].

### Step 2:

To a stirred solution of tert-butyl 1-(cyclopropylmethyl)-2-(7-fluoro-5-(methoxycarbonyl)-1-methyl-1H-benzo[d]imidazol-2-yl)-7-oxo-7,8-dihydropyrrolo[2,3-e]indole-6(1H)-carboxylate (50 mg, 0.09 mmol) in MeOH (3 mL) was added LiOH (19 mg, 0.46 mmol) in H₂O (3 mL). The mixture was stirred at 50° C. with microwave irradiation for 1 h, then filtered and the filtrate was concentrated in vacuo to give 2-(6-(tert-butoxycarbonyl)-1-(cyclopropylmethyl)-7-oxo-1,6,7,8-tetrahydropyrrolo[2,3-e]indol-2-yl)-7-fluoro-1-methyl-1H-benzo[d]imidazole-5-carboxylic acid (25 mg) as a gray oil, which was used without further purification. LC/MS (ESI⁺): m/z 519.2 [(M+H)⁺].

### Step 3:

A mixture of 2-(6-(tert-butoxycarbonyl)-1-(cyclopropylmethyl)-7-oxo-1,6,7,8-tetrahydropyrrolo[2,3-e]indol-2-yl)-7-fluoro-1-methyl-1H-benzo[d]imidazole-5-carboxylic acid (25 mg, 0.04 mmol), DIPEA (18 mg, 0.14 mmol), HATU (23 mg, 0.06 mmol), tert-butyl ((1R,4R,7R)-2-azabicyclo[2.2.1]heptan-7-yl)carbamate (10 mg, 0.04 mmol) in DMF (1.0 mL) was stirred at RT for 0.5 h and monitored by LC/MS. Upon completion, the reaction mixture was concentrated in vacuo to afford tert-butyl 2-(5-((1R,4R,7R)-7-((tert-butoxycarbonyl)amino)-2-azabicyclo[2.2.1]heptane-2-carbonyl)-7-fluoro-1-methyl-1H-benzo[d]imidazol-2-yl)-1-(cyclopropylmethyl)-7-oxo-7,8-dihydropyrrolo[2,3-e]indole-6(1H)-carboxylate (20 mg) as a light-yellow semi-solid, which was used without further purification. LC/MS (ESI⁺): m/z 613.2 [(M+H)⁺].

2) To a stirred solution of tert-butyl 2-(5-((1R,4R,7R)-7-((tert-butoxycarbonyl)amino)-2-azabicyclo[2.2.1]heptane-2-carbonyl)-7-fluoro-1-methyl-1H-benzo[d]imidazol-2-yl)-1-(cyclopropylmethyl)-7-oxo-7,8-dihydropyrrolo[2,3-e]indole-6(1H)-carboxylate (20 mg, 0.02 mmol) in MeOH was added 4 M HCl in dioxane (0.5 mL) and the resulting mixture was stirred at RT for 0.5 h. The mixture was concentrated in vacuo, basified with saturated Na₂CO₃ solution to pH=8, extracted with DCM (30 mL×3). The combined organic layers were dried over anhydrous sodium sulfate, filtered, and concentrated in vacuo. The residue was purified by flash column chromatography on silica gel (eluting with 0-8% MeOH in DCM) to give 2-(5-((1R,4R,7R)-7-amino-2-azabicyclo[2.2.1]heptane-2-carbonyl)-7-fluoro-1-methyl-1H-benzo[d]imidazol-2-yl)-1-(cyclopropylmethyl)-6,8-dihydropyrrolo[2,3-e]indol-7(1H)-one (0.3 mg, 0.58 μmol, 2.0% yield) as a light-yellow solid. LC/MS (ESI⁺): m/z 513.2 [(M+H)⁺].

### Example 146 Synthesis of 2-(5-((1R,4R,7R)-7-amino-2-azabicyclo[2.2.1]heptane-2-carbonyl)-7-fluoro-1-methyl-1H-benzo[d]imidazol-2-yl)-1-(cyclopropylmethyl)-6-methyl-6,8-dihydropyrrolo[2,3-e]indol-7(1H)-one

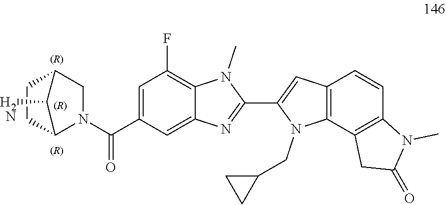

Prepared in analogous manner as for Example 145. LC/MS (ESI⁺): m/z 527.3 [(M+H)⁺].

### Example 147 Synthesis of ((1R,4R,7R)-7-amino-2-azabicyclo[2.2.1]heptan-2-yl)(2-(1-(cyclopropylmethyl)-1,6,7,8-tetrahydropyrrolo[3,2-g]indol-2-yl)-7-fluoro-1-methyl-1H-benzo[d]imidazol-5-yl)methanone

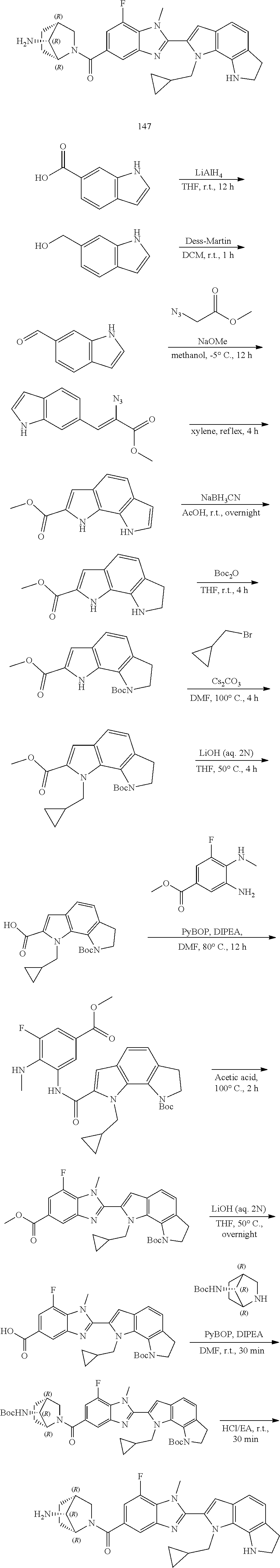

### Step 1:

To a stirred solution of 1H-indole-6-carboxylic acid (15 g, 93.08 mmol) in anhydrous THF (400 mL) was added LiAlH₄ (6.31 g, 186.15 mmol) in portions under N₂ atmosphere, and the resulting mixture was stirred at RT overnight. After completion, the mixture was cooled to 0° C., and EA (100 mL) was carefully added, followed by methanol (20 mL) and water (20 mL). The mixture was stirred for 30 min then filtered through celite. The filtrate was concentrated in vacuo, then diluted with EA (500 mL), washed with brine (2×100 mL), dried over MgSO₄, and concentrated in vacuo. The residue was purified by flash column chromatography on silica gel (eluting with 0 to 80% EtOAc in PE) to afford 1H-indol-6-ylmethanol (10 g, 67.95 mmol, 73.1% yield) as a brown oil. LC/MS (ESI⁺): m/z 147.8 [(M+H)⁺].

### Step 2:

A mixture of Dess-Martin periodinane (28.82 g, 67.95 mmol) and 1H-indol-6-ylmethanol (10 g, 67.95 mmol) in dichloromethane (70 mL) was stirred at RT for 1 h. Sodium hydroxide solution (5 M, 20 mL) was added, and the mixture was stirred at RT for 30 min. The organic layer was separated and washed with water (50 mL), brine (50 mL), dried over MgSO₄ and concentrated in vacuo. The residue was purified by flash column chromatography on silica gel (eluting with 0 to 50% EtOAc in PE) to give 1H-indole-6-carbaldehyde (5 g, 34.45 mmol, 50.1% yield) as a white solid. LC/MS (ESI⁺): m/z 145.9 [(M+H)⁺].

### Step 3:

To a cooled (−5° C.) solution of sodium methanolate (7.44 g, 137.78 mmol) in methanol (70 mL) was dropwise added a solution of 1H-indole-6-carbaldehyde (5 g, 34.45 mmol) and methyl 2-azidoacetate (15.86 g, 137.78 mmol) in methanol over 1 h. The resulting mixture was stirred at −5° C. for 4 h, then the heterogeneous mixture was diluted with 70 mL of water and filtered. The filtrate was washed with 30 mL water, dried over Na₂SO₄, and concentrated in vacuo to afford the desired product methyl (Z)-2-azido-3-(1H-indol-6-yl)prop-2-enoate (3 g) as a yellow solid, which was used without further purification.

### Step 4:

The suspension of methyl (Z)-2-azido-3-(1H-indol-6-yl)prop-2-enoate (3 g, 12.38 mmol) in xylene (20 mL) was stirred at 150° C. for 4 h before cooled to RT, filtered and dried in vacuo to afford methyl 1,8-dihydropyrrolo[3,2-g]indole-2-carboxylate (1.5 g) as a yellow solid, which was used without further purification. LC/MS (ESI⁺): m/z 214.8 [(M+H)⁺].

### Step 5:

To a stirred solution of methyl 1,8-dihydropyrrolo[3,2-g]indole-2-carboxylate (1.5 g, 7.00 mmol) in acetic acid (15 mL) was added sodium cyanoborohydride (1.32 g, 21.01 mmol) in portions and the resulting suspension was stirred at RT overnight. The mixture was concentrated, redissolved in EtOAc (300 mL) washed sequentially with 1 M NaOH (2×200 mL) and brine (100 mL), dried over MgSO₄, filtered, and concentrated in vacuo. The residue was purified by flash column chromatography on silica gel (eluting with 0 to 100% EtOAc in PE) to give methyl 1,6,7,8-tetrahydropyrrolo[3,2-g]indole-2-carboxylate (1 g, 4.62 mmol, 66.1% yield) as a white solid. LC/MS (ESI⁺): m/z 216.8 [(M+H)⁺].

### Step 6:

A mixture of methyl 1,6,7,8-tetrahydropyrrolo[3,2-g]indole-2-carboxylate (1 g, 4.62 mmol) and di-tert-butyl dicarbonate (1.11 g, 5.09 mmol) in THF (20 mL) was stirred at RT for 3 h. Upon completion, solvent was removed in vacuo. The residue was purified by flash column chromatography on silica gel (eluting with EA in PE 0 to 50%) to give 1-(tert-butyl) 7-methyl 3,8-dihydropyrrolo[3,2-g]indole-1,7(2H)-dicarboxylate (1.2 g, 3.79 mmol, 82.1% yield) as a white solid. LC/MS (ESI⁺): m/z 316.8 [(M+H)⁺].

### Step 7:

A mixture of 1-(tert-butyl) 7-methyl 3,8-dihydropyrrolo[3,2-g]indole-1,7(2H)-dicarboxylate (1.2 g, 3.79 mmol), bromomethyl cyclopropane (614.52 mg, 4.55 mmol) and cesium carbonate (3.71 g, 11.38 mmol) in DMF (10 mL) was stirred at 100° C. for 4 h under N₂ atmosphere. The mixture was partitioned between EtOAc and brine. The combined organic layers were dried over MgSO₄ and concentrated in vacuo. The residue was purified by flash column chromatography on silica gel (eluting with EA in PE 0 to 50%) to give 1-(tert-butyl) 7-methyl 8-(cyclopropylmethyl)-3,8-dihydropyrrolo[3,2-g]indole-1,7(2H)-dicarboxylate (800 mg, 2.16 mmol, 56.1% yield) as a white solid. LC/MS (ESI⁺): m/z 370.8 [(M+H)⁺].

### Step 8:

To a stirred solution of 1-(tert-butyl) 7-methyl 8-(cyclopropylmethyl)-3,8-dihydropyrrolo[3,2-g]indole-1,7(2H)-dicarboxylate (800 mg, 2.16 mmol) in THF/methanol (5 mL) mixed solvents was added LiOH (aq. 2 M, 4 mL). The resulting mixture was stirred at 50° C. for 4 h and monitored by LC/MS until full conversion of the starting material. The mixture was acidified by 2 mol/L HCl. Then solvent was removed in vacuo and the residue was purified by prep-HPLC to give the desired product 8-(tert-butoxycarbonyl)-1-(cyclopropylmethyl)-1,6,7,8-tetrahydropyrrolo[3,2-g]indole-2-carboxylic acid (650 mg, 1.82 mmol, 84.1% yield) as a white solid. LC/MS (ESI⁺): m/z 356.8 [(M+H)⁺].

### Step 9:

A mixture of 8-(tert-butoxycarbonyl)-1-(cyclopropylmethyl)-1,6,7,8-tetrahydropyrrolo[3,2-g]indole-2-carboxylic acid (800 mg, 2.24 mmol), methyl 3-amino-5-fluoro-4-(methylamino)benzoate (533.83 mg, 2.69 mmol), PyBOP (1.40 g, 2.69 mmol), and DIPEA (870.27 mg, 6.73 mmol) in DMF (15 mL) was stirred overnight at 80° C. under N₂ atmosphere. Upon completion, the solution was diluted with EtOAc, washed with water and brine, dried over anhydrous Na₂SO₄ and concentrated in vacuo. This residue was purified by flash column chromatography on silica gel (0 to 50% EtOAc in PE) to give tert-butyl 8-(cyclopropylmethyl)-7-((3-fluoro-5-(methoxycarbonyl)-2-(methylamino)phenyl)carbamoyl)-3,8-dihydropyrrolo[3,2-g]indole-1(2H)-carboxylate tert-butyl 1-(cyclopropylmethyl)-2-[[3-fluoro-5-methoxycarbonyl-2-(methylamino)phenyl]carbamoyl]-6,7-dihydropyrrolo[3,2-g]indole-8-carboxylate (400 mg, 745.44 μmol, 33.1% yield) as a colorless oil. LC/MS (ESI⁺): m/z 536.8 [(M+H)⁺].

### Step 10:

A mixture of tert-butyl 1-(cyclopropylmethyl)-2-[[3-fluoro-5-methoxycarbonyl-2-(methylamino)phenyl]carbamoyl]-6,7-dihydropyrrolo[3,2-g]indole-8-carboxylate (400 mg, 745.44 μmol) was dissolved in acetic acid (10 mL) and stirred at 100° C. for 2 h. Cooled the reaction to RT, removed the solvent in vacuo. The residue was purified by flash column chromatography on silica gel (eluting with 0 to 80% EtOAc in PE) to give tert-butyl 1-(cyclopropylmethyl)-2-(7-fluoro-5-methoxycarbonyl-1-methyl-benzimidazol-2-yl)-6,7-dihydropyrrolo[3,2-g]indole-8-carboxylate (300 mg, 578.51 μmol, 77.1% yield) as a white solid. LC/MS (ESI⁺): m/z 518.7 [(M+H)⁺].

### Step 11:

To a stirred solution of tert-butyl 1-(cyclopropylmethyl)-2-(7-fluoro-5-methoxycarbonyl-1-methyl-benzimidazol-2-yl)-6,7-dihydropyrrolo[3,2-g]indole-8-carboxylate (50 mg, 96.42 μmol) in THF/methanol (2 mL) mixed solvents was added LiOH (aq. 2 M, 2 mL). The resulting mixture was stirred at 50° C. overnight. The mixture was acidified with 2 M HCl. Upon completion, the mixture was concentrated in vacuo and the residue was purified by prep-HPLC to give the product 2-[8-tert-butoxycarbonyl-1-(cyclopropylmethyl)-6,7-dihydropyrrolo[3,2-g]indol-2-yl]-7-fluoro-1-methyl-benzimidazole-5-carboxylic acid (40 mg, 79.28 μmol, 82.1% yield) as a yellow solid. LC/MS (ESI⁺): m/z 504.8 [(M+H)⁺].

### Step 12:

A mixture of 2-[8-tert-butoxycarbonyl-1-(cyclopropylmethyl)-6,7-dihydropyrrolo[3,2-g]indol-2-yl]-7-fluoro-1-methyl-benzimidazole-5-carboxylic acid (40 mg, 79.28 μmol), tert-butyl N-[(1R,4R,7R)-2-azabicyclo[2.2.1]heptan-7-yl]carbamate (16.83 mg, 79.28 μmol), HATU (30.14 mg, 79.28 μmol), and DIPEA (30.74 mg, 237.83 μmol) in DMF (5 mL) was stirred at RTfor 30 min and monitored by LC/MS until full conversion of the starting material. The reaction was diluted with EtOAc (50 mL) and washed with water (25 mL). The organic layer was dried over sodium sulfate, filtered, and concentrated in vacuo. The crude material was purified by flash column chromatography on silica gel (eluting with EA in PE 0 to 100%) and prep-HPLC to give tert-butyl 2-[5-[(1R,4R,7R)-7-(tert-butoxycarbonylamino)-2-azabicyclo[2.2.1]heptane-2-carbonyl]-7-fluoro-1-methyl-benzimidazol-2-yl]-1-(cyclopropylmethyl)-6,7-dihydropyrrolo[3,2-g]indole-8-carboxylate (45 mg, 64.39 μmol, 81.1% yield) as a white solid. LC/MS (ESI⁺): m/z 698.7 [(M+H)⁺].

### Step 13:

To a stirred solution of tert-butyl 2-[5-[(1R,4R,7R)-7-(tert-butoxycarbonylamino)-2-azabicyclo [2.2.1]heptane-2-carbonyl]-7-fluoro-1-methyl-benzimidazol-2-yl]-1-(cyclopropylmethyl)-6,7-dihydropyrrolo[3,2-g]indole-8-carboxylate (45 mg, 64.39 μmol) in EA (3 mL) was added HCl in EA (3 mL). The resulting mixture was stirred at RT for 30 min and monitored by LC/MS until full conversion of the starting material. Then solvent was removed in vacuo and the residue was purified by prep-HPLC to afford [(1R,4R,7R)-7-amino-2-azabicyclo [2.2.1]heptan-2-yl]-[2-[1-(cyclopropylmethyl)-7,8-dihydro-6H-pyrrolo[3,2-g]indol-2-yl]-7-fluoro-1-methyl-benzimidazol-5-yl]methanone (25 mg, 50.14 μmol, 77.1% yield) as a yellow solid. LC/MS (ESI⁺): m/z 498.8 [(M+H)⁺]. ¹H NMR (400 MHz, DMSO-d₆) δ 7.69-7.58 (m, 1H), 7.28-7.18 (m, 1H), 7.00 (d, J=7.9 Hz, 1H), 6.95 (d, J=5.4 Hz, 2H), 5.62-5.50 (m, 1H), 4.43 (d, J=7.0 Hz, 2H), 4.06-4.03 (m, 3H), 3.72 (s, 1H), 3.56 (t, J=8.7 Hz, 2H), 3.50 (d, J=11.4 Hz, 1H), 3.19 (s, 1H), 3.08-3.02 (m, 3H), 2.21 (s, 1H), 1.95 (d, J=9.2 Hz, 2H), 1.71 (t, J=9.3 Hz, 1H), 1.45-1.38 (m, 1H), 0.97 (dd, J=10.1, 4.8 Hz, 1H), 0.19 (dd, J=8.1, 1.7 Hz, 2H), −0.06 (q, J=4.8 Hz, 2H).

### Example 148 Synthesis of ((1R,4R,7R)-7-amino-2-azabicyclo[2.2.1]heptan-2-yl)(2-(1-(cyclopropylmethyl)-1,8-dihydropyrrolo[3,2-g]indol-2-yl)-7-fluoro-1-methyl-1H-benzo[d]imidazol-5-yl)methanone

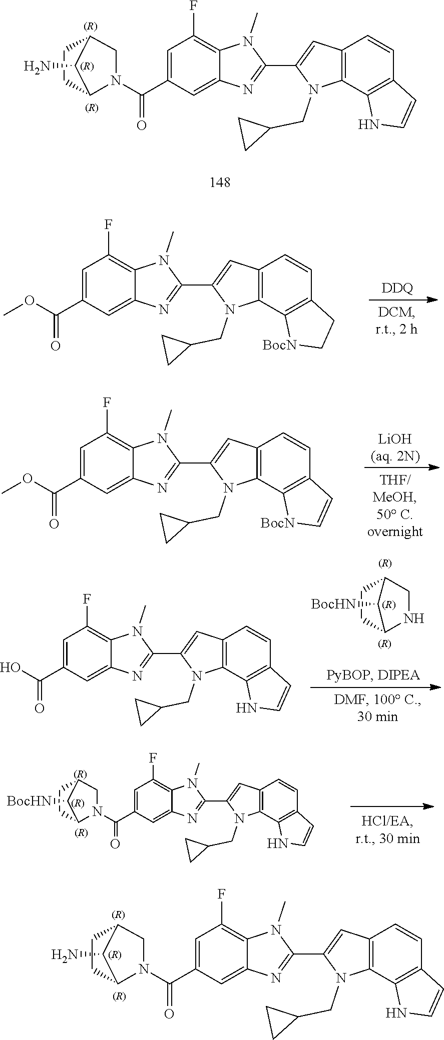

### Step 1:

To a stirred solution of tert-butyl 1-(cyclopropylmethyl)-2-(7-fluoro-5-methoxycarbonyl-1-methyl-benzimidazol-2-yl)-6,7-dihydropyrrolo[3,2-g]indole-8-carboxylate (50 mg, 96.42 μmol, intermediate of example 147) in dichloromethane (10 mL) at RT was added 2,3-Dichloro-5,6-dicyano-1,4-benzoquinone (21.89 mg, 96.42 μmol). After 2 h, the reaction was quenched by addition of brine and NaHCO₃. The resulting mixture was extracted with CH₂Cl₂, and the combined organic layers were washed with water followed by brine. The organic phase was dried over MgSO₄ and concentrated in vacuo. The residue was purified by flash column chromatography on silica gel (eluting with 0 to 30% EtOAc in PE) to give tert-butyl 1-(cyclopropylmethyl)-2-(7-fluoro-5-methoxycarbonyl-1-methyl-benzimidazol-2-yl)pyrrolo[3,2-g]indole-8-carboxylate (30 mg, 58.08 μmol, 60.2% yield) as a yellow solid. LC/MS (ESI⁺): m/z 516.7 [(M+H)⁺].

### Step 2:

To a stirred solution of tert-butyl 1-(cyclopropylmethyl)-2-(7-fluoro-5-methoxycarbonyl-1-methyl-benzimidazol-2-yl)pyrrolo[3,2-g]indole-8-carboxylate (30 mg, 58.08 μmol) in THF/methanol (3 mL) was added LiOH (aq. 2 M, 2 mL). The resulting mixture was stirred at 50° C. overnight. Upon completion, the mixture was acidified with 2 M HCl. Then solvent was removed in vacuo and the residue was purified by prep-HPLC to give the product 2-[1-(cyclopropylmethyl)-8H-pyrrolo[3,2-g]indol-2-yl]-7-fluoro-1-methyl-benzimidazole-5-carboxylic acid (20 mg, 49.70 μmol, 85.5% yield) as a yellow solid. LC/MS (ESI⁺): m/z 402.7 [(M+H)⁺].

### Step 3:

A mixture of 2-[1-(cyclopropylmethyl)-8H-pyrrolo[3,2-g]indol-2-yl]-7-fluoro-1-methyl-benzimidazole-5-carboxylic acid (20 mg, 49.70 μmol), tert-butyl N-[(1R,4R,7R)-2-azabicyclo[2.2.1]heptan-7-yl]carbamate (10.55 mg, 49.70 μmol), HATU (18.90 mg, 49.70 μmol), and DIPEA (19.27 mg, 149.10 μmol) in DMF (5 mL) was stirred at 100° C. for 30 min. Then the mixture was diluted with EtOAc (50 mL) and washed with water (25 mL). The organic layer was dried over sodium sulfate, filtered, and concentrated in vacuo. The residue was purified by flash column chromatography on silica gel (eluting with 0 to 100% EtOAc in petro ether) and prep-HPLC to give tert-butyl N-[(1R,4R,7R)-2-[2-[1-(cyclopropylmethyl)-8H-pyrrolo[3,2-g]indol-2-yl]-7-fluoro-1-methyl-benzimidazole-5-carbonyl]-2-azabicyclo[2.2.1]heptan-7-yl]carbamate (25 mg, 41.90 μmol, 84.1% yield) as a white solid. LC/MS (ESI⁺): m/z 596.7 [(M+H)⁺].

### Step 4:

To a stirred solution of tert-butyl N-[(1R,4R,7R)-2-[2-[1-(cyclopropylmethyl)-8H-pyrrolo[3,2-g]indol-2-yl]-7-fluoro-1-methyl-benzimidazole-5-carbonyl]-2-azabicyclo[2.2.1]heptan-7-yl]carbamate (25 mg, 41.90 μmol) in EA (3 mL) was added 4.0 M HCl in EA (2 mL). The resulting mixture was stirred at RT for 30 min. Upon completion, solvent was removed in vacuo and the residue was purified by prep-HPLC to afford [(1R,4R,7R)-7-amino-2-azabicyclo[2.2.1]heptan-2-yl]-[2-[1-(cyclopropylmethyl)-8H-pyrrolo[3,2-g]indol-2-yl]-7-fluoro-1-methyl-benzimidazol-5-yl]methanone (10 mg, 20.14 μmol, 48.1% yield) as a yellow solid. LC/MS (ESI⁺): m/z 496.8 [(M+H)⁺]. ¹H NMR (400 MHz, DMSO-d₆) δ 11.40 (s, 1H), 8.17 (s, 1H), 7.59 (d, J=1.2 Hz, 1H), 7.37-7.27 (m, 4H), 7.21 (d, J=11.9 Hz, 1H), 7.14 (s, 1H), 6.60 (t, J=2.3 Hz, 1H), 4.80 (d, J=7.0 Hz, 2H), 4.10 (d, J=2.9 Hz, 3H), 3.07 (d, J=11.0 Hz, 2H), 2.22 (s, 1H), 1.95 (s, 2H), 1.72 (d, J=8.0 Hz, 2H), 1.44 (d, J=7.5 Hz, 2H), 1.00 (s, 1H), 0.22 (d, J=8.0 Hz, 2H), −0.02 (d, J=5.0 Hz, 2H).

### Example 149 Synthesis of ((1R,4R,7R)-7-amino-2-azabicyclo[2.2.1]heptan-2-yl)(2-(6-chloro-1-(cyclopropylmethyl)-1,8-dihydropyrrolo[3,2-g]indol-2-yl)-7-fluoro-1-methyl-1H-benzo[d]imidazol-5-yl)methanone

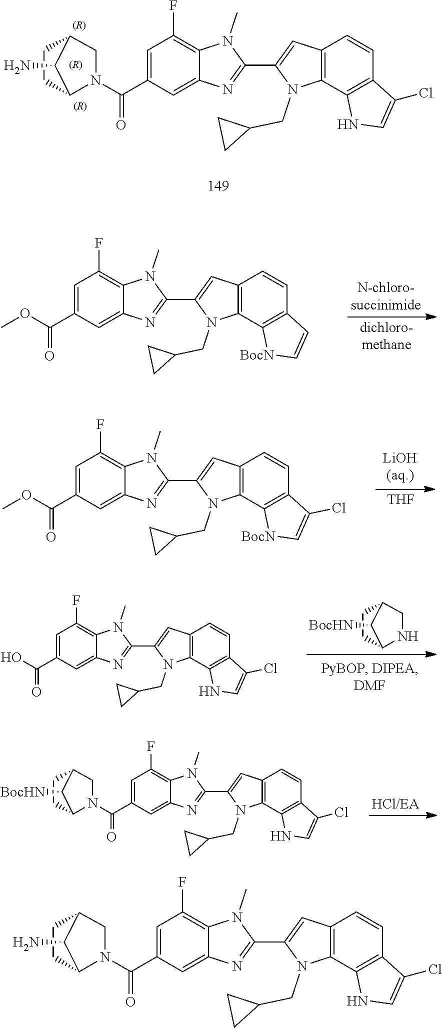

### Step 1:

To a stirred solution of tert-butyl 1-(cyclopropylmethyl)-2-(7-fluoro-5-methoxycarbonyl-1-methyl-benzimidazol-2-yl)pyrrolo[3,2-g]indole-8-carboxylate (100 mg, 193.59 μmol, intermediate of example 148) in dichloromethane (10 mL) was added N-chlorosuccinimide (25.85 mg, 193.59 μmol). The resulting mixture was stirred at 40° C. for 12 h. Upon completion, solvent was removed in vacuo and the residue was purified by flash column chromatography (0 to 50% EA in PE) to give tert-butyl 6-chloro-1-(cyclopropylmethyl)-2-(7-fluoro-5-methoxycarbonyl-1-methyl-benzimidazol-2-yl)pyrrolo[3,2-g]indole-8-carboxylate (80 mg, 75.1% yield) as a white solid. LC/MS (ESI⁺): m/z 550.6 [(M+H)⁺].

### Step 2:

To a stirred solution of tert-butyl 6-chloro-1-(cyclopropylmethyl)-2-(7-fluoro-5-methoxycarbonyl-1-methyl-benzimidazol-2-yl)pyrrolo[3,2-g]indole-8-carboxylate (30 mg, 54.45 μmol) in THF (2 mL) was added lithium hydroxide (2 M, 3 mL). The resulting mixture was stirred at RT for 30 min and monitored by LC/MS until full conversion of the starting material. HCl (2 M) was added to neutralize the solution. Then solvent was removed in vacuo and the residue was purified by prep-HPLC to give the desired product 2-[6-chloro-1-(cyclopropylmethyl)-8H-pyrrolo[3,2-g]indol-2-yl]-7-fluoro-1-methyl-benzimidazole-5-carboxylic acid (20 mg, 84.1% yield) as a yellow solid. LC/MS (ESI⁺): m/z 436.7 [(M+H)⁺].

### Step 3:

A mixture of 2-[6-chloro-1-(cyclopropylmethyl)-8H-pyrrolo[3,2-g]indol-2-yl]-7-fluoro-1-methyl-benzimidazole-5-carboxylic acid (20 mg, 45.78 μmol), tert-butyl N-[(1R,4R,7R)-2-azabicyclo[2.2.1]heptan-7-yl]carbamate (9.72 mg, 45.78 μmol), 1H-Benzotriazol-1-yloxytripyrrolidinophosphonium Hexafluorophosphate (23.82 mg, 45.78 μmol) and N,N-diisopropylethylamine (17.75 mg, 137.34 μmol) in DMF (5 mL) was stirred at 30° C. and monitored by LC/MS until full conversion. The solution was diluted with EtOAc (50 mL) and washed with water (25 mL). The organic layer was dried over sodium sulfate, filtered, and concentrated in vacuo. The crude material was purified by flash column chromatography on silica gel (0 to 100% EA in PE) and prep-HPLC to give tert-butyl N-[(1R,4R,7R)-2-[2-[6-chloro-1-(cyclopropylmethyl)-8H-pyrrolo[3,2-g]indol-2-yl]-7-fluoro-1-methyl-benzimidazole-5-carbonyl]-2-azabicyclo[2.2.1]heptan-7-yl]carbamate (25 mg, 86.1% yield) as a white solid. LC/MS (ESI⁺): m/z 630.6 [(M+H)⁺].

### Step 4:

A solution of Tert-butyl N-[(1R,4R,7R)-2-[2-[6-chloro-1-(cyclopropylmethyl)-8H-pyrrolo[3,2-g]indol-2-yl]-7-fluoro-1-methyl-benzimidazole-5-carbonyl]-2-azabicyclo[2.2.1]heptan-7-yl]carbamate (25 mg, 39.61 μmol) in EtOAc (1 mL) was treated with 4.0 M HCl in EtOAc (3 mL) at RT for 30 min and monitored by LC/MS until full conversion of the starting material. Then solvent was removed in vacuo and the residue was purified by prep-HPLC to afford [(1R,4R,7R)-7-amino-2-azabicyclo[2.2.1]heptan-2-yl]-[2-[6-chloro-1-(cyclopropylmethyl)-8H-pyrrolo[3,2-g]indol-2-yl]-7-fluoro-1-methyl-benzimidazol-5-yl]methanone (3 mg, 14.1% yield) as a white solid. LC/MS (ESI⁺): m/z 530.7 [(M+H)⁺].

### Example 150 Synthesis of ((1R,4R,7R)-7-amino-2-azabicyclo[2.2.1]heptan-2-yl)(2-(9-(cyclopropylmethyl)-9H-imidazo[1,2-a]pyrrolo[2,3-c]pyridin-8-yl)-7-fluoro-1-methyl-1H-benzo[d]imidazol-5-yl)methanone

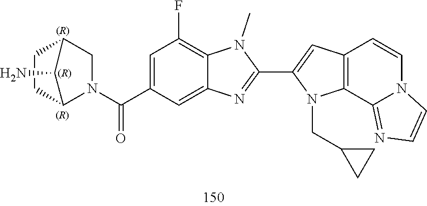

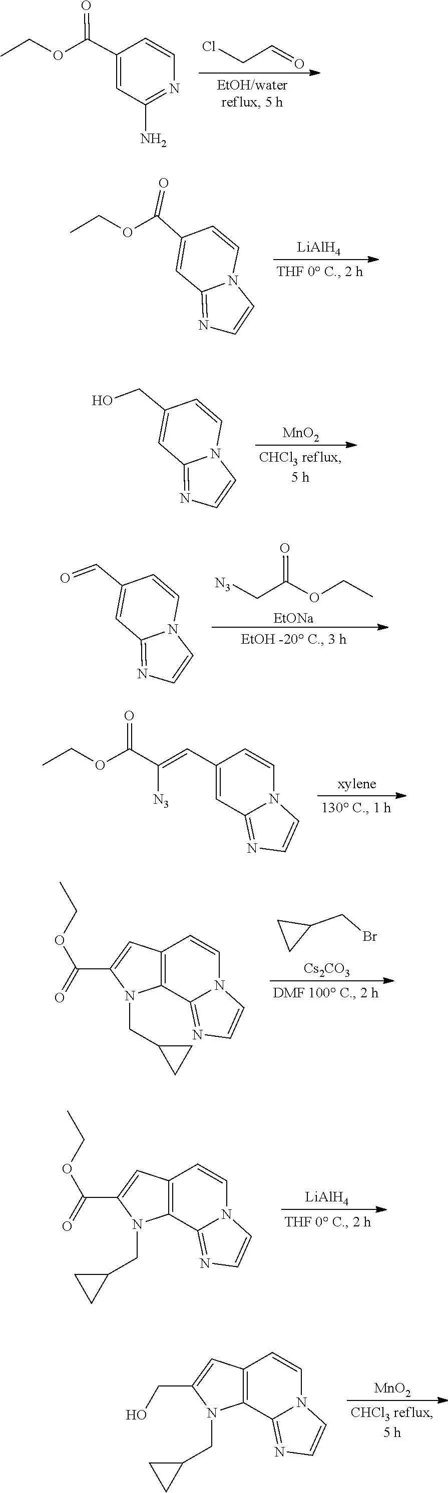

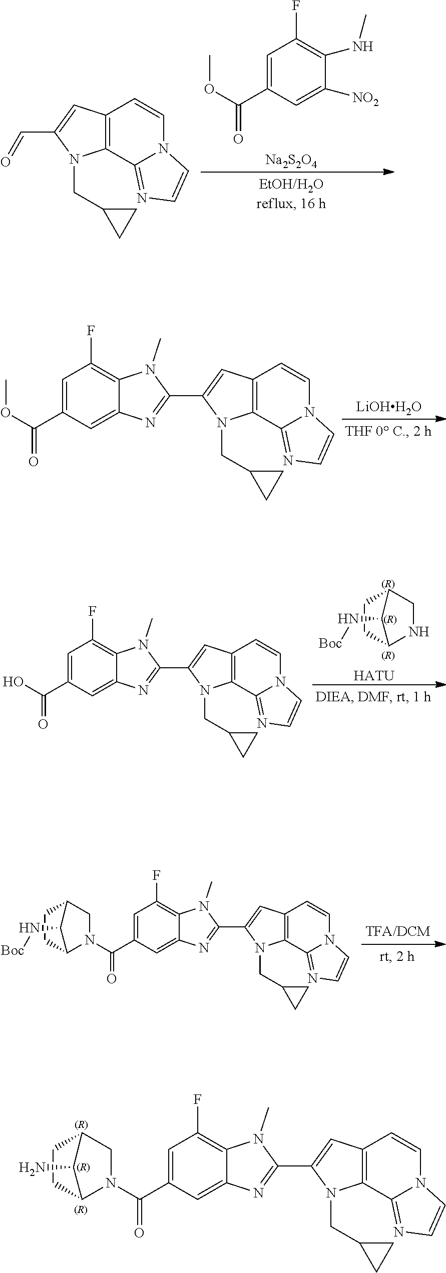

### Step 1:

A mixture of ethyl 2-aminopyridine-4-carboxylate (8.0 g, 48.14 mmol), chloroacetaldehyde (40% in water) (11.34 g, 57.77 mmol, 40% purity) and sodium bicarbonate (4.85 g, 57.77 mmol) in ethanol (20 mL) was stirred at 60° C. for 5 h. Then the mixture was concentrated and partitioned between EtOAc and water. The organic layer was washed with brine, dried over Na₂SO₄, concentrated and purified by flash chromatography on silica gel (eluting with 30%-50% EA in PE) to give ethyl imidazo[1,2-a]pyridine-7-carboxylate (8.0 g, 42.06 mmol, 87.1% yield) as a yellow solid. LC/MS (ESI⁺): m/z 190.9 [(M+H)⁺].

### Step 2:

To a stirred solution of ethyl imidazo[1,2-a]pyridine-7-carboxylate (8.0 g, 42.06 mmol) in THF (100 mL) at 0° C. was added LiAlH₄ (2.14 g, 63.09 mmol) in portions. The resulting mixture was stirred at 0° C. for 2 h and then quenched with 3% aqueous NaOH. The mixture was filtrated and the filtrate was concentrated to give imidazo[1,2-a]pyridin-7-ylmethanol (4.0 g, 27.00 mmol, 64.1% yield) as a yellow solid. LC/MS (ESI⁺): m/z 148.9 [(M+H)⁺].

### Step 3:

A mixture of imidazo[1,2-a]pyridin-7-ylmethanol (4.0 g, 27.00 mmol) and MnO₂ (12.01 g, 134.99 mmol) in CHCl₃ (30 mL) was stirred at reflux for 5 h. Then the mixture was filtered and the filtrate was concentrated and purified by flash chromatography on silica gel (eluting with 5% to 10% MeOH in DCM) to give imidazo[1,2-a]pyridine-7-carbaldehyde (2.0 g, 13.68 mmol, 50.1% yield) as an off-white solid. LC/MS (ESI⁺): m/z 146.9 [(M+H)⁺].

Step 4: To the mixture of imidazo[1,2-a]pyridine-7-carbaldehyde (1.0 g, 6.84 mmol) and ethyl 2-azidoacetate (4.42 g, 34.21 mmol) in ethanol (10 mL) at −20° C. was slowly added a solution of EtONa (11.09 g, 34.21 mmol) in ethanol (12.77 mL). The resulting mixture was slowly warmed to RT and stirred at RT for 3 h. Then the mixture was poured into saturated aqueous NH₄Cl, and precipitation was collected by filtration and dried under vacuum to give ethyl (Z)-2-azido-3-imidazo[1,2-a]pyridin-7-yl-prop-2-enoate (1.2 g, 4.66 mmol, 68.1% yield) as a light-yellow solid. LC/MS (ESI⁺): m/z 257.8 [(M+H)⁺].

### Step 5:

A mixture of ethyl (Z)-2-azido-3-imidazo[1,2-a]pyridin-7-yl-prop-2-enoate (1.2 g, 4.66 mmol) in xylene (15 mL) was heated to 130° C., and stirred for 1 h. Then the mixture was cooled down and concentrated in vacuo. The residue was purified by flash chromatography on silica gel (eluting with 10% to 20% MeOH in DCM) to give ethyl 3,6,12-triazatricyclo[7.3.0.02,6]dodeca-1(9), 2,4,7,10-pentaene-11-carboxylate (240 mg, 1.05 mmol, 22.4% yield) as a yellow oil. LC/MS (ESI⁺): m/z 229.8 [(M+H)⁺].

### Step 6:

A mixture of ethyl 3,6,12-triazatricyclo[7.3.0.02,6]dodeca-1(9), 2,4,7,10-pentaene-11-carboxylate (240 mg, 1.05 mmol), bromomethylcyclopropane (169.61 mg, 1.26 mmol) and Cs₂CO₃ (511.68 mg, 1.57 mmol) in DMF (5 mL) was stirred at 100° C. for 2 h. Then the mixture was partitioned between EA (3×15 mL) and water. The combined organic layers were washed with brine, dried over Na₂SO₄, concentrated and purified by flash chromatography on silica gel (eluting with 30% to 50% EA in PE) to give ethyl 12-(cyclopropylmethyl)-3,6,12-triazatricyclo[7.3.0.02,6]dodeca-1(9), 2,4,7,10-pentaene-11-carboxylate (270 mg, 952.97 μmol, 91.1% yield) as a yellow oil. LC/MS (ESI⁺): m/z 283.8 [(M+H)⁺].

### Step 7:

To a stirred solution of ethyl 12-(cyclopropylmethyl)-3,6,12-triazatricyclo[7.3.0.02,6]dodeca-1(9), 2,4,7,10-pentaene-11-carboxylate (270 mg, 952.97 μmol) in THF (5 mL) at 0° C. was added LiAlH₄ (64.65 mg, 1.91 mmol) in portions. The resulting mixture was stirred at 0° C. for 2 h and then quenched with 3% aqueous NaOH. The mixture was filtered and the filtrate was concentrated in vacuo to give [12-(cyclopropylmethyl)-3,6,12-triazatricyclo[7.3.0.02,6]dodeca-1(9), 2,4,7,10-pentaen-11-yl]methanol (200 mg, 828.89 μmol, 86.1% yield) as a colorless oil. LC/MS (ESI⁺): m/z 241.9 [(M+H)⁺].

### Step 8:

A mixture of [12-(cyclopropylmethyl)-3,6,12-triazatricyclo[7.3.0.02,6]dodeca-1(9), 2,4,7,10-pentaen-11-yl]methanol (200 mg, 828.89 μmol) and MnO₂ (368.66 mg, 4.14 mmol) in CHCl₃ (5 mL) was stirred at reflux for 5 h. Then the mixture was filtered and the filtrate was concentrated to give 12-(cyclopropylmethyl)-3,6,12-triazatricyclo[7.3.0.02,6]dodeca-1(9), 2,4,7,10-pentaene-11-carbaldehyde (180 mg, 752.28 μmol, 90.7% yield) as a yellow oil. LC/MS (ESI⁺): m/z 239.8 [(M+H)⁺].

### Step 9:

To the mixture of 12-(cyclopropylmethyl)-3,6,12-triazatricyclo[7.3.0.02,6]dodeca-1(9), 2,4,7,10-pentaene-11-carbaldehyde (180 mg, 752.28 μmol) and methyl 3-fluoro-4-(methylamino)-5-nitro-benzoate (143.04 mg, 626.90 μmol) in ethanol (6 mL) was added a solution of sodium dithionite (545.74 mg, 3.13 mmol) in water (2 mL). The mixture was stirred at reflux overnight. The mixture was concentrated in vacuo and the residue was partitioned between EA (3×15 mL) and water. The combined organic layers were washed with brine, dried over Na₂SO₄, concentrated and purified by flash chromatography on silica gel (eluting with 30% to 50% EA in PE) to give methyl 2-[12-(cyclopropylmethyl)-3,6,12-triazatricyclo[7.3.0.02,6]dodeca-1(9), 2,4,7,10-pentaen-11-yl]-7-fluoro-1-methyl-benzimidazole-5-carboxylate (200 mg, 479.12 μmol, 76.1% yield) as a yellow solid. LC/MS (ESI⁺): m/z 417.8 [(M+H)⁺].

### Step 10:

To the mixture of methyl 2-[12-(cyclopropylmethyl)-3,6,12-triazatricyclo[7.3.0.02,6]dodeca-1(9), 2,4,7,10-pentaen-11-yl]-7-fluoro-1-methyl-benzimidazole-5-carboxylate (100 mg, 239.56 μmol) in methanol/THF/water (5 mL, 2:2:1) mixed solvents was added lithium hydroxide monohydrate (50.26 mg, 1.20 mmol). The resulting mixture was stirred at RT for 2 h. Upon completion, the mixture was acidified with 4 M HCl in dioxane to pH=1, and then concentrated to give 2-[12-(cyclopropylmethyl)-3,6,12-triazatricyclo[7.3.0.02,6]dodeca-1(9), 2,4,7,10-pentaen-11-yl]-7-fluoro-1-methyl-benzimidazole-5-carboxylic acid (90 mg, 223.10 μmol, 93.1% yield) as a yellow solid. LC/MS (ESI⁺): m/z 403.7 [(M+H)⁺].

### Step 11:

To a stirred solution of 2-[12-(cyclopropylmethyl)-3,6,12-triazatricyclo[7.3.0.02,6]dodeca-1(9), 2,4,7,10-pentaen-11-yl]-7-fluoro-1-methyl-benzimidazole-5-carboxylic acid (80 mg, 198.31 μmol) in DMF (4 mL) was successively added HATU (98.02 mg, 257.80 μmol), DIPEA (76.89 mg, 594.93 μmol) and tert-Butyl (1R,4R,7R)-2-azabicyclo[2.2.1]heptan-7-ylcarbamate (50.52 mg, 237.97 μmol). The resulting mixture was stirred at RT for 1 h. Then the mixture was partitioned between EA (3×10 mL) and water. The combined organic layers were washed with brine, dried over Na₂SO₄, concentrated and purified by flash chromatography (eluting with 10% to 20% MeOH in DCM) to give tert-butyl N-[(1R,4R,7R)-2-[2-[12-(cyclopropylmethyl)-3,6,12-triazatricyclo[7.3.0.02,6]dodeca-1(9), 2,4,7,10-pentaen-11-yl]-7-fluoro-1-methyl-benzimidazole-5-carbonyl]-2-azabicyclo[2.2.1]heptan-7-yl]carbamate (100 mg, 167.31 μmol, 84.1% yield) as a yellow solid. LC/MS (ESI⁺): m/z 597.7 [(M+H)⁺].

### Step 12:

To a stirred solution of tert-butyl N-[(1R,4R,7R)-2-[2-[12-(cyclopropylmethyl)-3,6,12-triazatricyclo[7.3.0.02,6]dodeca-1(9), 2,4,7,10-pentaen-11-yl]-7-fluoro-1-methyl-benzimidazole-5-carbonyl]-2-azabicyclo[2.2.1]heptan-7-yl]carbamate (50 mg, 83.66 μmol) in DCM (2 mL) was added TFA (740.00 mg, 6.49 mmol, 0.5 mL) dropwise. The resulting mixture was stirred at RT for 1 h. Then the mixture was concentrated and purified by prep-HPLC to give [(1R,4R,7R)-7-amino-2-azabicyclo[2.2.1]heptan-2-yl]-[2-[12-(cyclopropylmethyl)-3,6,12-triazatricyclo[7.3.0.02,6]dodeca-1(9), 2,4,7,10-pentaen-1l-yl]-7-fluoro-1-methyl-benzimidazol-5-yl]methanone (17 mg, 34.17 μmol, 40.1% yield) as a white solid. LC/MS (ESI⁺): m/z 498.7 [(M+H)⁺]. ¹H NMR (400 MHz, DMSO-d₆) δ 8.22 (d, J=7.0 Hz, 1H), 7.99 (d, J=1.0 Hz, 1H), 7.64 (s, 1H), 7.51 (s, 1H), 7.33-7.25 (m, 1H), 7.20 (d, J=7.0 Hz, 1H), 7.17 (s, 1H), 5.06 (d, J=7.2 Hz, 2H), 4.10 (s, 3H), 3.91 (s, 1H), 3.70-3.52 (m, 2H), 3.48-3.32 (m, 3H), 3.12-3.10 (m, 1H), 2.36 (s, 1H), 1.93 (d, J=8.0 Hz, 1H), 1.82-1.80 (m, 1H), 1.52 (t, J=8.4 Hz, 1H), 1.27-1.14 (m, 1H), 0.26 (d, J=8.0 Hz, 2H), 0.19 (d, J=4.6 Hz, 2H).

### Example 151 Synthesis of ((1R,4R,7R)-7-amino-2-azabicyclo[2.2.1]heptan-2-yl)(2-(1-(cyclopropylmethyl)-6,7,8,9-tetrahydro-1H-pyrrolo[2,3-f]isoquinolin-2-yl)-7-fluoro-1-methyl-1H-benzo[d]imidazol-5-yl)methanone

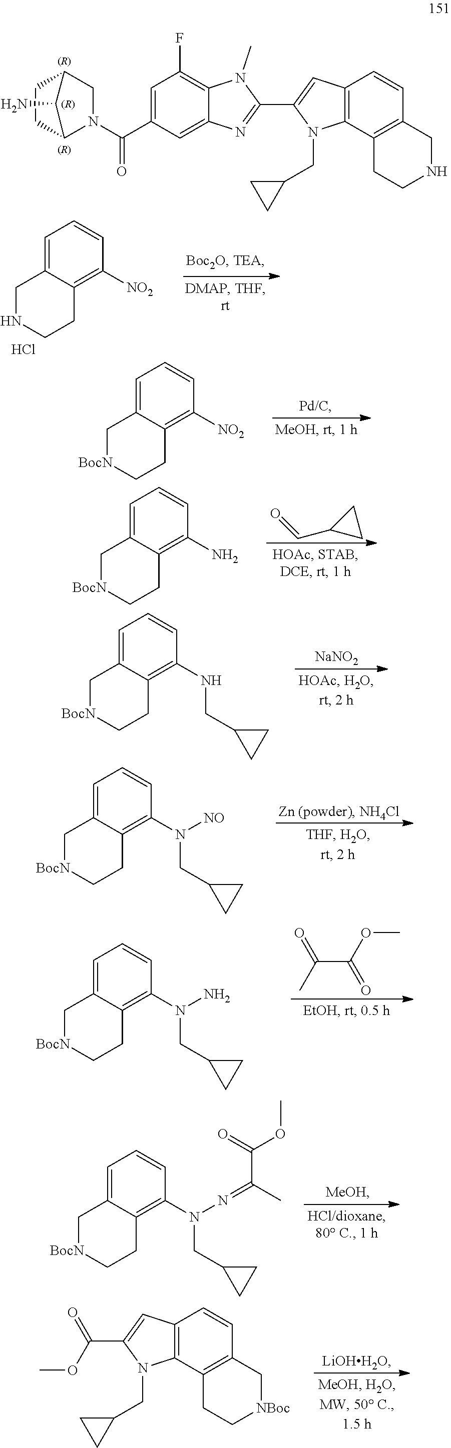

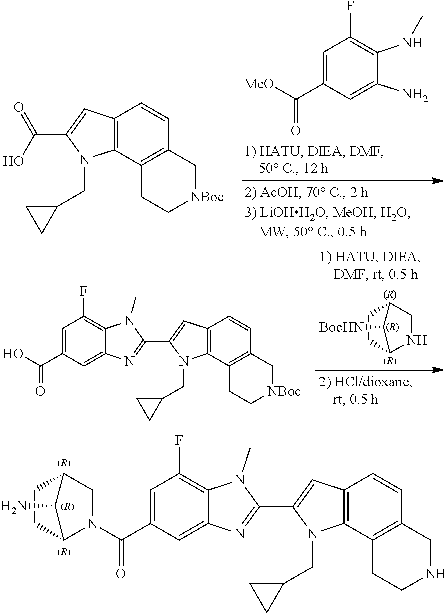

### Step 1:

To a stirred solution of 5-nitro-1,2,3,4-tetrahydroisoquinoline hydrochloride (3.00 g, 14.01 mmol), DMAP (85 mg, 0.70 mmol) and TEA (3.52 mL, 21.02 mmol) in THF (60 mL) was added Boc₂O (3.67 g, 16.81 mmol) and the resulting mixture was stirred at RT for 6 h. Solvent was removed in vacuo and the residue was purified by flash column chromatography on silica gel (eluting with 0-10% EA in PE) to give tert-butyl 5-nitro-3,4-dihydroisoquinoline-2(1H)-carboxylate (2.44 g, 8.77 mmol, 60.0% yield) as a yellow oil. LC/MS (ESI⁺): m/z 223.1 [(M-56+H)⁺].

### Step 2:

A mixture of tert-butyl 5-nitro-3,4-dihydroisoquinoline-2(1H)-carboxylate (2.44 g, 8.77 mmol) and 10 wt % Pd/C (1.22 g, 726.81 mmol) in MeOH (30 mL) under an atmosphere of H₂ (balloon) was stirred at RT for 3 h and monitored by LC/MS until the starting material was consumed. The reaction mixture was filtered through Celite, and the filtrate was evaporated in vacuo to afford tert-butyl 5-amino-3,4-dihydroisoquinoline-2(1H)-carboxylate (2.28 g, 9.18 mmol, 94.2% yield) as a white solid. LC/MS (ESI⁺): m/z 249.1 [(M+H)⁺].

### Step 3:

A solution of tert-butyl 5-amino-3,4-dihydroisoquinoline-2(1H)-carboxylate (2.28 g, 9.18 mmol), acetic acid (1.65 g, 27.56 mmol) and cyclopropanecarbaldehyde (514 mg, 7.35 mmol) in DCE (50 mL) was stirred at RT for 30 min before STAB (2.92 g, 13.78 mmol) was added in portions. The resulting mixture was stirred at RT for 0.5 h, then diluted with EA, washed with 10% KOH and brine, dried over Na₂SO₄, and concentrated in vacuo to give tert-butyl 5-((cyclopropylmethyl)amino)-3,4-dihydroisoquinoline-2(1H)-carboxylate (2.38 g) as a white semi-solid, which was used without further purification. LC/MS (ESI⁺): m/z 303.2 [(M+H)⁺].

### Step 4:

To a stirred solution of tert-butyl 5-((cyclopropylmethyl)amino)-3,4-dihydroisoquinoline-2(1H)-carboxylate (2.38 g, 7.87 mmol) in CH₃COOH (26 mL) was added and sodium nitrite (1.08 g, 15.75 mmol) at 0° C. and the resulting mixture was stirred at 25° C. for 2 h. Then the reaction mixture was diluted with DCM, washed with sat. NaHCO₃ and brine, dried over anhydrous Na₂SO₄, and concentrated in vacuo to give tert-butyl 5-((cyclopropylmethyl)(nitroso)amino)-3,4-dihydroisoquinoline-2(1H)-carboxylate (2.15 g) as a brown semi-solid, which was used without further purification in the next step. LC/MS (ESI⁺): m/z 332.1 [(M+H)⁺].

### Step 5:

To a stirred solution of tert-butyl 5-((cyclopropylmethyl)(nitroso)amino)-3,4-dihydroisoquinoline-2(1H)-carboxylate (2.15 g, 6.49 mmol) and NH₄Cl (1.30 g, 24.34 mmol) in THF/H₂O mixed solvents (30 mL, 2:1) at 0° C. was added zinc powder (1.27 g, 19.47 mmol) and the resulting mixture was stirred at RT for 2 h. Upon completion, the reaction mixture was filtered, and the filtrate was diluted with water (40 mL) and extracted with EtOAc (120 mL×2). The combined organic layers were dried over Na₂SO₄, filtered, and concentrated in vacuo. The residue was purified by flash column chromatography on silica gel (eluting with 0-12% EA in PE) to give tert-butyl 5-(1-(cyclopropylmethyl)hydrazineyl)-3,4-dihydroisoquinoline-2(1H)-carboxylate (514 mg, 1.55 mmol, 16.7% yield) as a yellow oil. LC/MS (ESI⁺): m/z 318.2 [(M+H)⁺].

### Step 6:

A mixture of tert-butyl 5-(1-(cyclopropylmethyl)hydrazineyl)-3,4-dihydroisoquinoline-2(1H)-carboxylate (514 mg, 1.55 mmol) and methyl 2-oxopropanoate (330 mg, 3.24 mmol) in EtOH (5.0 mL) was stirred at RT for 0.5 h, then concentrated to afford tert-butyl 5-(1-(cyclopropylmethyl)-2-(1-methoxy-1-oxopropan-2-ylidene)hydrazineyl)-3,4-dihydroisoquinoline-2(1H)-carboxylate (800 mg) as a yellow semi-solid, which was used without further purification in the next step. LC/MS (ESI⁺): m/z 402.2 [(M+H)⁺].

### Step 7:

To a stirred solution of tert-butyl 5-(1-(cyclopropylmethyl)-2-(1-methoxy-1-oxopropan-2-ylidene)hydrazineyl)-3,4-dihydroisoquinoline-2(1H)-carboxylate (800 mg, 1.99 mmol) in MeOH (10 mL) was added 4.0 M HCl in dioxane (10 mL). The resulting mixture was heated to 80° C. and stirred for 0.5 h. After cooling to RT, the mixture was concentrated in vacuo to afford 7-(tert-butyl) 2-methyl 1-(cyclopropylmethyl)-1,6,8,9-tetrahydro-7H-pyrrolo[2,3-f]isoquinoline-2,7-dicarboxylate (900 mg) as a purplish red solid, which was used without further purification in the next step. LC/MS (ESI⁺): m/z 385.2 [(M+H)⁺].

### Step 8:

To a stirred solution of 7-(tert-butyl) 2-methyl 1-(cyclopropylmethyl)-1,6,8,9-tetrahydro-7H-pyrrolo[2,3-f]isoquinoline-2,7-dicarboxylate (900 mg, 2.34 mmol) in MeOH (10 mL) was added a solution of LiOH (491 mg, 11.71 mmol) in H₂O (10 mL). The resulting mixture was stirred at 50° C. with microwave irradiation for 1 h, then filtered and concentrated in vacuo. The residue was purified with flash column chromatography on silica gel (eluting with 0-10% MeOH in DCM) to give 7-(tert-butoxycarbonyl)-1-(cyclopropylmethyl)-6,7,8,9-tetrahydro-1H-pyrrolo[2,3-f]isoquinoline-2-carboxylic acid (350 mg, 0.94 mmol, 51.8% yield) as a yellow oil. LC/MS (ESI⁺): m/z 379.1 [(M+H)⁺].

### Step 9:

1) A mixture of 7-(tert-butoxycarbonyl)-1-(cyclopropylmethyl)-6,7,8,9-tetrahydro-1H-pyrrolo[2,3-f]isoquinoline-2-carboxylic acid (110 mg, 0.75 mmol) in DMF (1 mL), DIPEA (114 mg, 0.89 mmol), HATU (146 mg, 0.38 mmol) and tert-butyl ((1R,4R,7R)-2-azabicyclo[2.2.1]heptan-7-yl)carbamate (88 mg, 0.44 mmol) was stirred at 50° C. for 12 h. Upon completion indicated by LC/MS, the mixture was concentrated in vacuo to afford tert-butyl 1-(cyclopropylmethyl)-2-((3-fluoro-5-(methoxycarbonyl)-2-(methylamino)phenyl)carbamoyl)-1,6,8,9-tetrahydro-7H-pyrrolo[2,3-f]isoquinoline-7-carboxylate as a crude product, which was used without further purification in the next step. LC/MS (ESI⁺): m/z 551.2 [(M+H)⁺].

2) A solution of tert-butyl 1-(cyclopropylmethyl)-2-((3-fluoro-5-(methoxycarbonyl)-2-(methylamino)phenyl)carbamoyl)-1,6,8,9-tetrahydro-7H-pyrrolo[2,3-f]isoquinoline-7-carboxylate (100 mg, 0.18 mmol) in CH₃COOH (2 mL) was stirred at 70° C. for 2.5 h. The mixture was concentrated in vacuo to afford tert-butyl 1-(cyclopropylmethyl)-2-(7-fluoro-5-(methoxycarbonyl)-1-methyl-1H-benzo[d]imidazol-2-yl)-1,6,8,9-tetrahydro-7H-pyrrolo[2,3-f]isoquinoline-7-carboxylate as a crude product, which was used without further purification in the next step. LC/MS (ESI⁺): m/z 533.2 [(M+H)⁺].

3) To a stirred solution of tert-butyl 1-(cyclopropylmethyl)-2-(7-fluoro-5-(methoxycarbonyl)-1-methyl-1H-benzo[d]imidazol-2-yl)-1,6,8,9-tetrahydro-7H-pyrrolo[2,3-f]isoquinoline-7-carboxylate (50 mg, 0.09 mmol) in MeOH (10 mL) was added a solution of LiOH (19 mg, 0.47 mmol) in H₂O (10 mL). The mixture was stirred at 50° C. with microwave irradiation for 0.5 h, then filtered. The filtrate was concentrated in vacuo to give 2-(7-(tert-butoxycarbonyl)-1-(cyclopropylmethyl)-6,7,8,9-tetrahydro-1H-pyrrolo[2,3-f]isoquinolin-2-yl)-7-fluoro-1-methyl-1H-benzo[d]imidazole-5-carboxylic acid as a crude product, which was used without further purification in the next step. LC/MS (ESI⁺): m/z 519.2 [(M+H)⁺].

### Step 10:

1) A mixture of 2-(7-(tert-butoxycarbonyl)-1-(cyclopropylmethyl)-6,7,8,9-tetrahydro-1H-pyrrolo[2,3-f]isoquinolin-2-yl)-7-fluoro-1-methyl-1H-benzo[d]imidazole-5-carboxylic acid (40 mg, 0.08 mmol), DIPEA (30 mg, 0.23 mmol), HATU (38 mg, 0.1 mmol), tert-butyl ((1R,4R,7R)-2-azabicyclo[2.2.1]heptan-7-yl)carbamate (16 mg, 0.08 mmol) in DMF (1 mL) was stirred at RT for 0.5 h. Upon completion indicated by LC/MS, the mixture was concentrated in vacuo to afford tert-butyl 2-(5-((1R,4R,7R)-7-((tert-butoxycarbonyl)amino)-2-azabicyclo[2.2.1]heptane-2-carbonyl)-7-fluoro-1-methyl-1H-benzo[d]imidazol-2-yl)-1-(cyclopropylmethyl)-1,6,8,9-tetrahydro-7H-pyrrolo[2,3-f]isoquinoline-7-carboxylate as a crude product, which was used without further purification in the next step. LC/MS (ESI⁺): m/z 713.3 [(M+H)⁺].

2) To a solution tert-butyl 2-(5-((1R,4R,7R)-7-((tert-butoxycarbonyl)amino)-2-azabicyclo[2.2.1]heptane-2-carbonyl)-7-fluoro-1-methyl-1H-benzo[d]imidazol-2-yl)-1-(cyclopropylmethyl)-1,6,8,9-tetrahydro-7H-pyrrolo[2,3-f]isoquinoline-7-carboxylate (45 mg, 0.06 mmol) in dioxane was added 4.0 M HCl in dioxane (0.5 mL) and the resulting mixture was stirred at RT for 10 min. Upon completion indicated by LC/MS, the mixture was concentrated in vacuo, basified to pH=8 with saturated Na₂CO₃ solution and extracted with DCM (30 mL×3). The combined organic layers were dried over anhydrous sodium sulfate, filtered, and concentrated in vacuo. The residue was purified by flash column chromatography on silica gel (eluting with 0-15% MeOH in DCM) to give ((1R,4R,7R)-7-amino-2-azabicyclo[2.2.1]heptan-2-yl)(2-(1-(cyclopropylmethyl)-6,7,8,9-tetrahydro-1H-pyrrolo[2,3-f]isoquinolin-2-yl)-7-fluoro-1-methyl-1H-benzo[d]imidazol-5-yl)methanone (5.5 mg, 0.01 mmol, 23.0% yield) as a white solid. LC/MS (ESI⁺): m/z 513.2 [(M+H)⁺]. ¹H NMR (400 MHz, DMSO-d₆) δ 8.07-7.97 (m, 1H), 7.38-7.29 (m, 1H), 7.00 (d, J=11.8 Hz, 1H), 6.85-6.79 (m, 1H), 6.79-6.69 (m, 1H), 4.52-4.47 (m, 2H), 4.45-4.32 (m, 2H), 3.72-3.62 (m, 3H), 3.56-3.51 (m, 2H), 3.49 (s, 1H), 3.30-3.16 (m, 4H), 2.96 (s, 1H), 2.87-2.69 (m, 2H), 1.98 (s, 1H), 1.94-1.86 (m, 2H), 1.80-1.64 (m, 2H), 1.54-1.39 (m, 1H), 1.27-1.11 (m, 1H), 0.74-0.63 (m, 1H), −0.52-−0.61 (m, 2H).

### Example 152 Synthesis of ((1R,4R,7R)-7-amino-2-azabicyclo[2.2.1]heptan-2-yl)(2-(1-(cyclopropylmethyl)-7-methyl-6,7,8,9-tetrahydro-1H-pyrrolo[2,3-f]isoquinolin-2-yl)-7-fluoro-1-methyl-1H-benzo[d]imidazol-5-yl)methanone

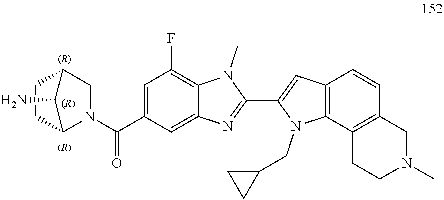

Prepared in analogous manner as for Example 151. LC/MS (ESI⁺): m/z 527.2 [(M+H)⁺]. ¹H NMR (400 MHz, DMSO-d₆) δ 7.73-7.20 (m, 3H), 7.17-6.96 (m, 1H), 6.83-6.63 (m, 1H), 4.47-4.22 (m, 2H), 4.09-3.76 (m, 3H), 3.73-3.39 (m, 3H), 3.45-3.29 (m, 2H), 3.00-2.61 (m, 4H), 2.61-2.51 (m, 1H), 2.28-1.96 (m, 3H), 1.83-1.44 (m, 3H), 1.35-1.15 (m, 2H), 0.66-0.55 (m, 1H), 0.41-0.11 (m, 2H), −0.24-−0.68 (m, 2H).

### Example 153 Synthesis of ((1R,4R,7R)-7-amino-2-azabicyclo[2.2.1]heptan-2-yl)(2-(1-(cyclopropylmethyl)-7-ethyl-6,7,8,9-tetrahydro-1H-pyrrolo[2,3-f]isoquinolin-2-yl)-7-fluoro-1-methyl-1H-benzo[d]imidazol-5-yl)methanone

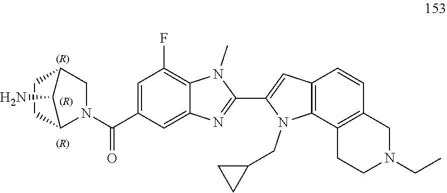

Prepared in analogous manner as for Example 151. LC/MS (ESI⁺): m/z 541.3 [(M+H)⁺].

### Example 154 Synthesis of ((1R,4R,7R)-7-amino-2-azabicyclo[2.2.1]heptan-2-yl)(2-(1-(cyclopropylmethyl)-7-isopropyl-6,7,8,9-tetrahydro-1H-pyrrolo[2,3-f]isoquinolin-2-yl)-7-fluoro-1-methyl-1H-benzo[d]imidazol-5-yl)methanone

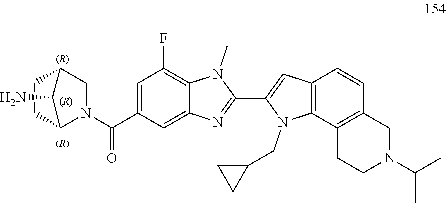

Prepared in analogous manner as for Example 151. LC/MS (ESI⁺): m/z 555.3 [(M+H)⁺].

### Example 155 Synthesis of ((1R,4R,7R)-7-amino-2-azabicyclo[2.2.1]heptan-2-yl)(2-(7-cyclopropyl-1-(cyclopropylmethyl)-6,7,8,9-tetrahydro-1H-pyrrolo[2,3-f]isoquinolin-2-yl)-7-fluoro-1-methyl-1H-benzo[d]imidazol-5-yl)methanone

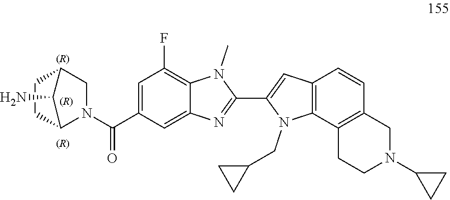

Prepared in analogous manner as for Example 151. LC/MS (ESI⁺): m/z 553.3 [(M+H)⁺]. ¹H NMR (400 MHz, DMSO-d₆) δ 7.77-7.52 (m, 2H), 7.49-7.45 (m, 1H), 7.16-6.99 (m, 1H), 6.86-6.70 (m, 1H), 4.42-4.25 (m, 2H), 3.85-3.55 (m, 3H), 3.68-3.45 (m, 2H), 3.45-3.30 (m, 2H), 2.94-2.65 (m, 3H), 2.45-2.28 (m, 3H), 1.85-1.55 (m, 2H), 1.45-1.35 (m, 2H), 1.20-0.94 (m, 2H), 0.87-0.61 (m, 1H), 0.75-0.56 (m, 1H), 0.49-0.01 (m, 4H), −0.13-−0.68 (m, 3H).

### Example 156 Synthesis of 1-(2-(5-((1R,4R,7R)-7-amino-2-azabicyclo[2.2.1]heptane-2-carbonyl)-7-fluoro-1-methyl-1H-benzo[d]imidazol-2-yl)-1-(cyclopropylmethyl)-1,6,8,9-tetrahydro-7H-pyrrolo[2,3-f]isoquinolin-7-yl)ethan-1-one

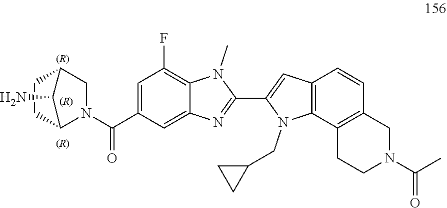

Prepared in analogous manner as for Example 151. LC/MS (ESI⁺): m/z 555.3 [(M+H)⁺]. ¹H NMR (400 MHz, DMSO-d₆) δ 8.02 (s, 1H), 7.65-7.50 (m, 2H), 7.33-7.21 (m, 1H), 7.07-6.95 (m, 2H), 4.80 (s, 1H), 4.74 (s, 1H), 4.58-4.50 (m, 2H), 4.15-4.00 (m, 3H), 3.82-3.75 (m, 2H), 3.75-3.61 (m, 1H), 3.51-3.45 (m, 1H), 3.21-3.15 (m, 1H), 3.10-2.94 (m, 2H), 2.22 (s, 1H), 2.13 (d, J=9.6 Hz, 3H), 2.02-1.56 (m, 5H), 1.47-1.34 (m, 1H), 1.27-1.17 (m, 1H), 1.01-0.8 (m, 1H), 0.23 (d, J=8.2 Hz, 2H), −0.29 (dd, J=8.8, 4.9 Hz, 2H).

### Example 157 Synthesis of ((1R,4R,7R)-7-amino-2-azabicyclo[2.2.1]heptan-2-yl)(2-(8-(cyclopropylmethyl)-1,8-dihydropyrrolo[3,2-g]indazol-7-yl)-7-fluoro-1-methyl-1H-benzo[d]imidazol-5-yl)methanone

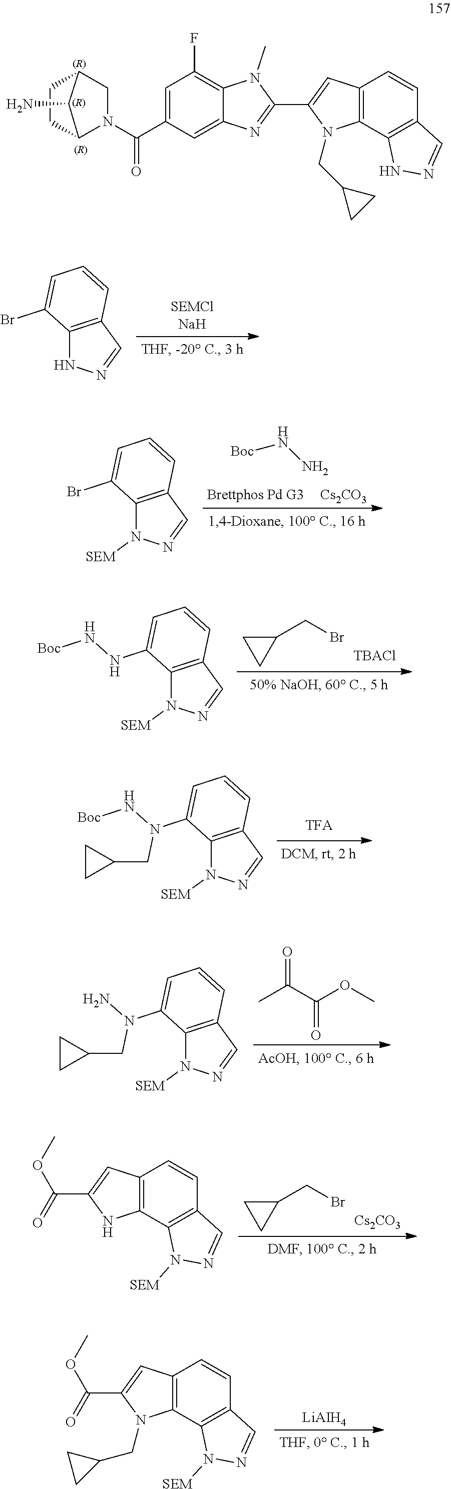

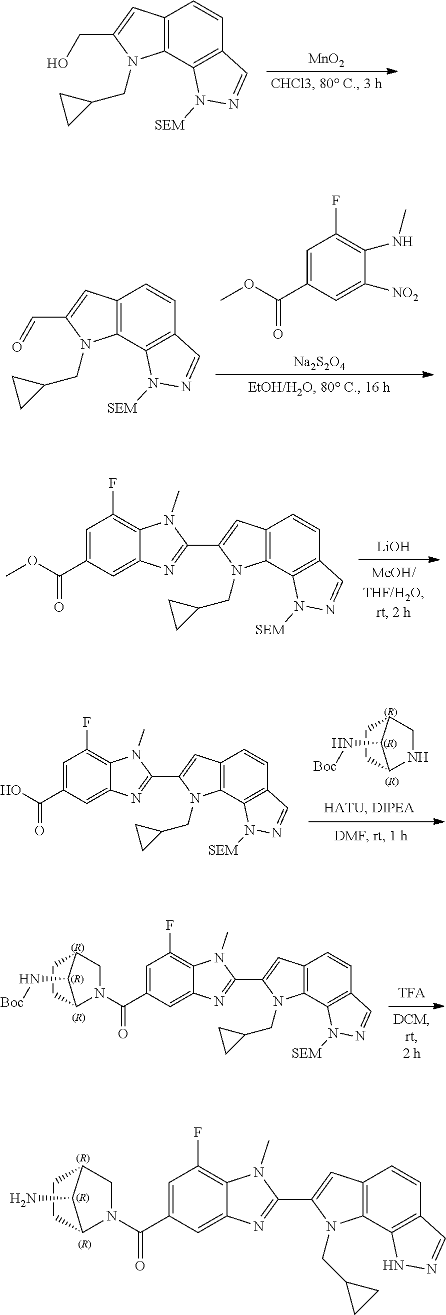

### Step 1:

To a mixture of 7-bromo-1H-indazole (1.0 g, 5.08 mmol) in anhydrous THF (15 mL) was added NaH (291.70 mg, 7.61 mmol, 60% dispersion in mineral oil) at −20° C. in portions. The mixture was stirred at −20° C. for 45 min, and then 2-(trimethylsilyl)ethoxymethyl chloride (930.78 mg, 5.58 mmol) was added slowly. The suspension was stirred at −20° C. for 3 h. The mixture was quenched with water, extracted with EA (3×30 mL). The combined organic layers were washed with brine, dried over Na₂SO₄, concentrated in vacuo and purified by flash chromatography on silica gel (eluting with 5-10% EA in PE) to give 2-[(7-bromoindazol-1-yl)methoxy]ethyl-trimethyl-silane (600 mg, 1.83 mmol, 36.1% yield) as a colorless oil. LC/MS (ESI⁺): m/z 326.7 [(M+H)⁺].

### Step 2:

A mixture of 2-[(7-bromoindazol-1-yl)methoxy]ethyl-trimethyl-silane (600 mg, 1.83 mmol) and tert-butyl carbazate (605.70 mg, 4.58 mmol), BrettPhos-Pd-G3 (16.62 mg, 18.33 μmol) and cesium carbonate (895.95 mg, 2.75 mmol) in dioxane (10 mL) was stirred at 100° C. for 16 h under N₂ atmosphere. Then the mixture was diluted with brine and extracted with EA (15 mL×3). The organic layer was dried over Na₂SO₄, concentrated and purified by flash chromatography on silica gel (eluting with 0-10% EA in PE) to give tert-butyl N-[[1-(2-trimethylsilylethoxymethyl)indazol-7-yl]amino]carbamate (320 mg, 845.35 μmol, 46.1% yield) as an off-white solid. LC/MS (ESI⁺): m/z 378.9 [(M+H)⁺].

### Step 3:

A mixture of tert-butyl N-[[1-(2-trimethylsilylethoxymethyl)indazol-7-yl]amino]carbamate (700 mg, 1.85 mmol), (bromomethyl)cyclopropane (299.58 mg, 2.22 mmol) and tetrabutyl ammonium chloride (25.70 mg, 92.46 μmol) in NaOH (50%, 8 mL) was stirred at 60° C. for 5 h. Then the mixture was extracted with DCM (3×15 mL), and the organic layer was washed with water and brine, dried over Na₂SO₄, concentrated and purified by flash chromatography on silica gel (eluting with 5-15% EA in PE) to give tert-butyl N-[cyclopropylmethyl-[1-(2-trimethylsilylethoxymethyl)indazol-7-yl]amino]carbamate (500 mg, 1.16 mmol, 62.5% yield) as a yellow oil. LC/MS (ESI⁺): m/z 432.8 [(M+H)⁺].

### Step 4:

To a mixture of tert-butyl N-[cyclopropylmethyl-[1-(2-trimethylsilylethoxymethyl)indazol-7-yl]amino]carbamate (500 mg, 1.16 mmol) in DCM (4 mL) was added TFA (1.48 g, 12.98 mmol, 1.0 mL). The mixture was stirred at RT for 2 h. Then 10% K₂CO₃ aq. was added to basify the mixture to pH=9. The mixture was extracted with DCM, and the organic layer was washed with brine, dried over Na₂SO₄, and concentrated in vacuo to give 1-(cyclopropylmethyl)-1-[1-(2-trimethylsilylethoxymethyl)indazol-7-yl]hydrazine (380 mg, 1.14 mmol, 98.8% yield) as a yellow oil. LC/MS (ESI⁺): m/z 332.8 [(M+H)⁺].

### Step 5:

A mixture of 1-(cyclopropylmethyl)-1-[1-(2-trimethylsilylethoxymethyl)indazol-7-yl]hydrazine (380 mg, 1.14 mmol) and methyl pyruvate (116.67 mg, 1.14 mmol) in AcOH (4 mL) was heated to 100° C. and stirred for 6 h. Then the mixture was concentrated and basified with 10% K₂CO₃ to pH=9. The mixture was extracted with EA (3×15 mL), and the organic layer was washed with brine, dried over Na₂SO₄, concentrated and purified by flash chromatography on silica gel (eluting with 10-25% EA in PE) to give methyl 1-(2-trimethylsilylethoxymethyl)-8H-pyrrolo[3,2-g]indazole-7-carboxylate (180 mg, 521.03 μmol, 45.5% yield) as a yellow oil. LC/MS (ESI⁺): m/z 345.8 [(M+H)⁺].

### Step 6:

A mixture of methyl 1-(2-trimethylsilylethoxymethyl)-8H-pyrrolo[3,2-g]indazole-7-carboxylate (180 mg, 521.03 μmol), (bromomethyl)cyclopropane (105.51 mg, 781.55 μmol), and cesium carbonate (339.52 mg, 1.04 mmol) in DMF (3 mL) was stirred at 100° C. for 2 h. Then the mixture was extracted with EA (3×10 mL) and the combined organic layers were washed with brine, dried over Na₂SO₄, concentrated in vacuo and purified by flash chromatography on silica gel (eluting with 15-30% EA in PE) to give methyl 8-(cyclopropylmethyl)-1-(2-trimethylsilylethoxymethyl)pyrrolo[3,2-g]indazole-7-carboxylate (180 mg, 450.50 μmol, 86.4% yield) as a yellow oil. LC/MS (ESI⁺): m/z 399.7 [(M+H)⁺].

### Step 7:

To a mixture of methyl 8-(cyclopropylmethyl)-1-(2-trimethylsilylethoxymethyl)pyrrolo[3,2-g]indazole-7-carboxylate (90 mg, 225.25 μmol) in THF (3 mL) was added LiAlH₄ (11.46 mg, 337.87 μmol) at 0° C. and the mixture was stirred at 0° C. for 1 h. Upon completion, the mixture was quenched with 10% aqueous Na₂SO₄, then filtered and the filtrate was concentrated to give [8-(cyclopropylmethyl)-1-(2-trimethylsilylethoxymethyl)pyrrolo[3,2-g]indazol-7-yl]methanol (70 mg, 188.40 μmol, 83.6% yield) as a white solid. LC/MS (ESI⁺): m/z 371.8 [(M+H)⁺].

### Step 8:

To a mixture of [8-(cyclopropylmethyl)-1-(2-trimethylsilylethoxymethyl)pyrrolo[3,2-g]indazol-7-yl]methanol (70 mg, 188.40 μmol) in CHCl₃ (3 mL) was added manganese dioxide (83.79 mg, 942.01 μmol), and the mixture was stirred at 80° C. for 3 h. Then the mixture was filtered off and the filtrate was concentrated to give crude 8-(cyclopropylmethyl)-1-(2-trimethylsilylethoxymethyl)pyrrolo[3,2-g]indazole-7-carbaldehyde (69 mg, 189.43 μmol, 99.1% yield) as a brown oil. LC/MS (ESI⁺): m/z 370.0 [(M+H)⁺].

### Step 9:

To a mixture of 8-(cyclopropylmethyl)-1-(2-trimethylsilylethoxymethyl)pyrrolo[3,2-g]indazole-7-carbaldehyde (70 mg, 189.43 μmol) and methyl 3-fluoro-4-(methylamino)-5-nitro-benzoate (38.90 mg, 170.49 μmol) in EtOH/H₂O mixed solvents (4 mL, 3:1) was added disodium hydrosulfite (164.90 mg, 947.14 μmol), and the mixture was stirred at 80° C. for 16 h. Then the mixture was concentrated, and the residue was dispensed between EA (3×10 mL) and water. The organic layer was washed with brine, dried over Na₂SO₄, concentrated and purified by flash chromatography on silica gel (eluting with 40-60% EA in PE) to give methyl 2-[8-(cyclopropylmethyl)-1-(2-trimethylsilylethoxymethyl)pyrrolo[3,2-g]indazol-7-yl]-7-fluoro-1-methyl-benzimidazole-5-carboxylate (80 mg, 146.07 μmol, 77.1% yield) as a yellow oil. LC/MS (ESI⁺): m/z 547.7 [(M+H)⁺].

### Step 10:

To a mixture of methyl 2-[8-(cyclopropylmethyl)-1-(2-trimethylsilylethoxymethyl)pyrrolo[3,2-g]indazol-7-yl]-7-fluoro-1-methyl-benzimidazole-5-carboxylate (40 mg, 73.03 μmol) in methanol/THF/water (5 mL, 2:2:1) mixed solvents was added lithium hydroxide monohydrate (15.32 mg, 365.17 μmol). The mixture was stirred at RT for 2 h. Then the mixture was acidified to pH=3 with aqueous HCl, extracted with DCM. The organic layer was washed with brine, dried over Na₂SO₄, and concentrated in vacuo to give 2-[8-(cyclopropylmethyl)-1-(2-trimethylsilylethoxymethyl)pyrrolo[3,2-g]indazol-7-yl]-7-fluoro-1-methyl-benzimidazole-5-carboxylic acid (28 mg, 52.47 μmol, 71.8% yield) as a yellow solid. LC/MS (ESI⁺): m/z 533.7 [(M+H)⁺].

### Step 11:

A mixture of 2-[8-(cyclopropylmethyl)-1-(2-trimethylsilylethoxymethyl)pyrrolo[3,2-g]indazol-7-yl]-7-fluoro-1-methyl-benzimidazole-5-carboxylic acid (30 mg, 56.21 μmol), HATU (27.79 mg, 73.08 μmol), DIPEA (21.80 mg, 168.64 μmol), tert-Butyl (1R,4R,7R)-2-azabicyclo[2.2.1]heptan-7-ylcarbamate (14.32 mg, 67.46 μmol) in DMF (2 mL) was stirred at RT for 1 h. The mixture was extracted with EA (3×10 mL) and the organic layers were combined and washed with brine, dried over Na₂SO₄, and purified by prep-TLC to give tert-butyl N-[(1R,4R,7R)-2-[2-[8-(cyclopropylmethyl)-1-(2-trimethylsilylethoxymethyl)pyrrolo[3,2-g]indazol-7-yl]-7-fluoro-1-methyl-benzimidazole-5-carbonyl]-2-azabicyclo[2.2.1]heptan-7-yl]carbamate (20 mg, 27.47 μmol, 48.8% yield) as a white solid. LC/MS (ESI⁺): m/z 727.7 [(M+H)⁺].

### Step 12:

To a mixture of tert-butyl N-[(1R,4R,7R)-2-[2-[8-(cyclopropylmethyl)-1-(2-trimethylsilylethoxymethyl)pyrrolo[3,2-g]indazol-7-yl]-7-fluoro-1-methyl-benzimidazole-5-carbonyl]-2-azabicyclo[2.2.1]heptan-7-yl]carbamate (20 mg, 27.47 μmol) in DCM (1 mL) was added TFA (1.48 g, 12.98 mmol, 1.0 mL). The mixture was stirred at RT for 2 h. Then the mixture was basified with 10% K₂CO₃ to pH=9, then extracted with DCM. The combined organic layers were washed with brine, dried over Na₂SO₄, concentrated and purified by prep-TLC to give [(1R,4R,7R)-7-amino-2-azabicyclo[2.2.1]heptan-2-yl]-[2-[8-(cyclopropylmethyl)-1H-pyrrolo[3,2-g]indazol-7-yl]-7-fluoro-1-methyl-benzimidazol-5-yl]methanone (5.0 mg, 10.05 μmol, 36.5% yield) as a white solid. LC/MS (ESI⁺): m/z 497.7 [(M+H)⁺]. ¹H NMR (400 MHz, DMSO-d₆) δ 8.36-8.27 (m, 1H), 7.73-7.62 (m, 1H), 7.42 (d, J=14.2 Hz, 2H), 7.24-7.21 (m, 2H), 5.01-4.87 (m, 2H), 4.11 (s, 3H), 3.76 (m, 1H), 3.52 (d, J=11.6 Hz, 1H), 3.23 (s, 1H), 3.09 (d, J=11.0 Hz, 1H), 2.24 (s, 1H), 2.00-1.96 (m, 3H), 1.76-1.72 (m, 1H), 1.46-1.45 (m, 1H), 1.26-1.24 (m, 3H), 0.26 (d, J=7.8 Hz, 2H), 0.13 (s, 2H).

### Example 158 Synthesis of ((1R,4R,7R)-7-amino-2-azabicyclo[2.2.1]heptan-2-yl)(2-(9-(cyclopropylmethyl)-1,2,3,9-tetrahydro-[1,4]oxazino[2,3-g]indol-8-yl)-7-fluoro-1-methyl-1H-benzo[d]imidazol-5-yl)methanone

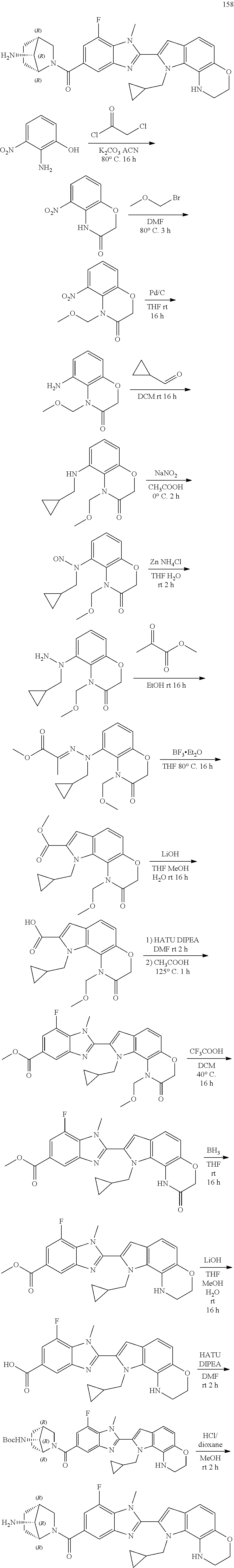

### Step 1:

A mixture of 2-amino-3-nitrophenol (10.0 g, 64.88 mmol), 2-chloroacetyl chloride (7.69 g, 68.13 mmol) and potassium carbonate (26.90 g, 194.65 mmol) in anhydrous ACN (150 mL) was stirred at 80° C. for 16 h. At this time LC/MS analysis showed that the reaction was completed. After cooling to RT, the reaction mixture was carefully poured into water (100 mL) with vigorous stirring, extracted with EA, washed with brine, and dried over anhydrous sodium sulfate. After filtration and evaporation of the solvent in vacuo, the residue was purified by flash column chromatography on silica gel (eluting with 0-8% EA in PE) to give 5-nitro-2H-benzo[b][1,4]oxazin-3(4H)-one (9.5 g, 48.93 mmol, 75.4% yield) as a yellow solid. LC/MS (ESI⁺): m/z 194.8 [(M+H)⁺].

### Step 2:

A mixture of 5-nitro-4H-1,4-benzoxazin-3-one (3.0 g, 15.45 mmol), DIPEA (5.99 g, 46.36 mmol) and bromo(methoxy)methane (3.86 g, 30.90 mmol) in anhydrous DMF (30 mL) was heated at 80° C. for 3 h. At this time LC/MS analysis showed that the reaction was completed. After cooling to RT, the reaction mixture was carefully poured into water (30 mL) with vigorous stirring, extracted with EA, washed with brine, and dried over anhydrous sodium sulfate. After filtration and evaporation of the solvent in vacuo, the residue was purified by flash column chromatography on silica gel (eluting with 0-10% EA in PE) to give 4-(methoxymethyl)-5-nitro-2H-benzo[b][1,4]oxazin-3(4H)-one (3.6 g, 15.11 mmol, 97.8% yield) as a yellow solid. LC/MS (ESI⁺): m/z 238.8 [(M+H)⁺].

### Step 3:

A mixture of 4-(methoxymethyl)-5-nitro-1,4-benzoxazin-3-one (3.6 g, 15.11 mmol) and 10 wt % Pd/C (360 mg) in MeOH (40 mL) under H₂ atmosphere was stirred at RT for 16 h and filtered. The filtrate was concentrated in vacuo to afford 5-amino-4-(methoxymethyl)-2H-benzo[b][1,4]oxazin-3(4H)-one (3.1 g, 14.89 mmol, 98.5% yield) as a brown solid. LC/MS (ESI⁺): m/z 208.8 [(M+H)⁺].

### Step 4:

To a stirred solution of 5-amino-4-(methoxymethyl)-1,4-benzoxazin-3-one (3.1 g, 14.89 mmol) in DCM (100 mL) was added cyclopropanecarbaldehyde (1.10 g, 15.63 mmol) at RT. The mixture was stirred at RT for 30 min and cooled down to 0° C. Then sodium triacetoxyborohyride (9.47 g, 44.67 mmol) was added to the above mixture in portions. The resulting mixture was stirred at RT for 16 h, then quenched with ice-water (40 mL) and extracted with DCM (100 mL×3). The combined organic layers were dried over anhydrous sodium sulfate, filtered, and concentrated. The residue was purified with flash column chromatography on silica gel (eluting with 0-5% MeOH in DCM) to give 5-((cyclopropylmethyl)amino)-4-(methoxymethyl)-2H-benzo[b][1,4]oxazin-3(4H)-one (2.5 g, 9.53 mmol, 64.0% yield) as a yellow solid. LC/MS (ESI⁺): m/z 262.8 [(M+H)⁺].

### Step 5:

To a stirred solution of 5-(cyclopropylmethylamino)-4-(methoxymethyl)-1,4-benzoxazin-3-one (2.5 g, 9.53 mmol) in CH₃COOH (30 mL) was added a solution of sodium nitrite (677.4 mg, 9.82 mmol) in H₂O (10 mL) at 0° C. The resulting mixture was stirred at 0° C. for 2 h and monitored by the LC/MS until the reaction was completed. The mixture was diluted with DCM, washed with brine, and dried over Na₂SO₄, filtered and concentrated in vacuo to give N-(cyclopropylmethyl)-N-(4-(methoxymethyl)-3-oxo-3,4-dihydro-2H-benzo[b][1,4]oxazin-5-yl)nitrous amide (2.7 g, 9.27 mmol, 97.2% yield) as a yellow solid. LC/MS (ESI⁺): m/z 291.8 [(M+H)⁺].

### Step 6:

To a stirred solution of N-(cyclopropylmethyl)-N-[4-(methoxymethyl)-3-oxo-1,4-benzoxazin-5-yl]nitrous amide (2.7 g, 9.27 mmol) in THF (30 mL) was added a solution of ammonium chloride (1.98 g, 37.07 mmol) in H₂O (30 mL), and then zinc (2.42 g, 37.07 mmol) was added at RT. The resulting mixture was stirred at RT for 2 h and monitored by the LC/MS until the reaction was completed. The mixture was filtered and the filtrate was extracted with EA (30 mL×2). The combined organic layers were dried over Na₂SO₄, filtered and concentrated in vacuo to afford 5-(1-(cyclopropylmethyl)hydrazineyl)-4-(methoxymethyl)-2H-benzo[b][1,4]oxazin-3(4H)-one (2.5 g) as a yellow solid, which was used in the next step directly without further purification.

### Step 7:

To a stirred solution of 5-[amino(cyclopropylmethyl)amino]-4-(methoxymethyl)-1,4-benzoxazin-3-one (2.5 g, 9.01 mmol) in EtOH (40 mL) was added methyl 2-oxopropanoate (920.3 mg, 9.01 mmol) under N₂ atmosphere. The resulting mixture was stirred at RT for 16 h, then concentrated in vacuo. The residue was slurried in a mixed solvent of DCM (12.5 mL, 5 v/w) and PE (62.5 mL, 25 v/w), then filtered and the filter cake was dried in vacuo to afford methyl (E)-2-(2-(cyclopropylmethyl)-2-(4-(methoxymethyl)-3-oxo-3,4-dihydro-2H-benzo[b][1,4]oxazin-5-yl)hydrazineylidene)propanoate (3.0 g, 8.30 mmol, 92.0% yield) as a yellow solid. LC/MS (ESI⁺): m/z 361.8 [(M+H)⁺].

### Step 8:

To a stirred solution of methyl (2E)-2-[cyclopropylmethyl-[4-(methoxymethyl)-3-oxo-1,4-benzoxazin-5-yl]hydrazono]propanoate (2.5 g, 6.92 mmol) in THF (20 mL) was added boron trifluoride diethyl etherate (2.95 g, 20.75 mmol) under N₂ atmosphere. The resulting mixture was heated to 80° C. and stirred for 16 h. After cooling to RT, the mixture was concentrated in vacuo. The residue was diluted with DCM, washed with water, and dried over Na₂SO₄. After filtration and evaporation of the solvent, the crude mixture was purified by flash column chromatography on silica gel (eluting with 0-5% MeOH in DCM) to give methyl 9-(cyclopropylmethyl)-1-(methoxymethyl)-2-oxo-1,2,3,9-tetrahydro-[1,4]oxazino[2,3-g]indole-8-carboxylate (625 mg, 1.81 mmol, 26.2% yield) as a yellow solid. LC/MS (ESI⁺): m/z 344.7 [(M+H)⁺].

### Step 9:

To a stirred solution of methyl 9-(cyclopropylmethyl)-1-(methoxymethyl)-2-oxo-pyrrolo[2,3-f][1,4]benzoxazine-8-carboxylate (625 mg, 1.81 mmol) in THF/MeOH mixed solvents (15 mL, 2:1) was added LiOH aqueous solution (1.0 M, 5.4 mL). The resulting mixture was stirred at RT for 1 h, then acidified to pH=5-6 with 3 M hydrochloric acid aqueous solution. The mixture was extracted with EA (20 mL×3) and the combined organic layers were dried over anhydrous sodium sulfate, filtered, and concentrated in vacuo to give 9-(cyclopropylmethyl)-1-(methoxymethyl)-2-oxo-pyrrolo[2,3-f][1,4]benzoxazine-8-carboxylic acid (600 mg, 1.82 mmol, 100.0% yield) as a white solid. LC/MS (ESI⁺): m/z 330.7 [(M+H)⁺].

### Step 10:

To a stirred solution of 9-(cyclopropylmethyl)-1-(methoxymethyl)-2-oxo-pyrrolo[2,3-f][1,4]benzoxazine-8-carboxylic acid (600 mg, 1.82 mmol) in DMF (10 mL) was successively added DIPEA (281.69 mg, 2.18 mmol), HATU (2.07 g, 5.45 mmol) and methyl 3-amino-5-fluoro-4-(methylamino)benzoate (377.99 mg, 1.91 mmol). The resulting mixture was stirred at RT for 2 h. Upon completion, the mixture was diluted with EA, washed with brine and dried over anhydrous sodium sulfate. After filtration and evaporation of the solvent in vacuo, the residue was redissolved in CH₃COOH (10 mL) and stirred at 125° C. for 1 h. After cooling to RT, the reaction mixture was concentrated in vacuo. The residue was diluted with EA (80 mL), washed with saturated Na₂CO₃ solution and dried over Na₂SO₄. After filtration and evaporation of the solvent in vacuo, the residue was purified by flash column chromatography on silica gel (eluting with 0-10% MeOH in DCM) to give methyl 2-(9-(cyclopropylmethyl)-1-(methoxymethyl)-2-oxo-1,2,3,9-tetrahydro-[1,4]oxazino[2,3-g]indol-8-yl)-7-fluoro-1-methyl-1H-benzo[d]imidazole-5-carboxylate (500 mg, 1.02 mmol, 55.8% yield) as a yellow solid. LC/MS (ESI⁺): m/z 492.6 [(M+H)⁺].

### Step 11:

To a stirred solution of methyl 2-[9-(cyclopropylmethyl)-1-(methoxymethyl)-2-oxo-pyrrolo[2,3-f][1,4]benzoxazin-8-yl]-7-fluoro-1-methyl-benzimidazole-5-carboxylate (500 mg, 1.02 mmol) in anhydrous DCM (10 mL) was added CF₃COOH (5 mL) dropwise at 0° C. The reaction mixture was stirred at 40° C. for 16 h and then concentrated in vacuo. The residue was purified by flash column chromatography on silica gel (eluting with 0-5% MeOH in DCM) to give methyl 2-[9-(cyclopropylmethyl)-2-oxo-1H-pyrrolo[2,3-f][1,4]benzoxazin-8-yl]-7-fluoro-1-methyl-benzimidazole-5-carboxylate (310 mg, 691.28 μmol, 68.0% yield) as a yellow solid. LC/MS (ESI⁺): m/z 448.6 [(M+H)⁺].

### Step 12:

To a stirred solution of methyl 2-[9-(cyclopropylmethyl)-2-oxo-1H-pyrrolo[2,3-f][1,4]benzoxazin-8-yl]-7-fluoro-1-methyl-benzimidazole-5-carboxylate (310 mg, 691.28 μmol) in anhydrous THF (3 mL) was slowly added borane tetrahydrofuran (1 M, 2.77 mL) at 0° C. The reaction mixture was stirred at RT for 16 h, then quenched with MeOH at 0° C. and concentrated in vacuo. The residue was diluted with 2 M HCl aqueous solution (3 mL), then stirred at RT for 1 h and basified with 4 M NaOH aqueous solution to pH ˜8. The resulting mixture was extracted with DCM (30 mL×3), and the combined organic layers were dried over anhydrous sodium sulfate. After filtration and evaporation of the solvent in vacuo, the residue was purified by flash column chromatography on silica gel (eluting with 0-5% MeOH in DCM) to give methyl 2-[9-(cyclopropylmethyl)-2,3-dihydro-1H-pyrrolo[2,3-f][1,4]benzoxazin-8-yl]-7-fluoro-1-methyl-benzimidazole-5-carboxylate (98 mg, 225.57 μmol, 32.6% yield) as a yellow solid. LC/MS (ESI⁺): m/z 434.7 [(M+H)⁺].

### Step 13:

To a stirred solution of methyl 2-[9-(cyclopropylmethyl)-2,3-dihydro-1H-pyrrolo[2,3-f][1,4]benzoxazin-8-yl]-7-fluoro-1-methyl-benzimidazole-5-carboxylate (98 mg, 225.57 μmol) in THF/MeOH mixed solvents (3 mL, 2:1) was added LiOH aqueous solution (1.0 M, 0.68 mL). The resulting mixture was stirred at RT for 16 h, and acidified to pH=5-6 with 3 M hydrochloric acid aqueous solution, then extracted with DCM (20 mL×3). The organic phase was dried over anhydrous sodium sulfate, filtered and concentrated in vacuo to give 2-[9-(cyclopropylmethyl)-2,3-dihydro-1H-pyrrolo[2,3-f][1,4]benzoxazin-8-yl]-7-fluoro-1-methyl-benzimidazole-5-carboxylic acid (85 mg, 202.17 μmol, 89.6% yield) as a white solid. LC/MS (ESI⁺): m/z 420.7 [(M+H)⁺].

### Step 14:

To a stirred solution 2-[9-(cyclopropylmethyl)-2,3-dihydro-1H-pyrrolo[2,3-f][1,4]benzoxazin-8-yl]-7-fluoro-1-methyl-benzimidazole-5-carboxylic acid (85 mg, 202.17 μmol) in DMF (2 mL) was successively added DIPEA (78.4 mg, 606.51 μmol), HATU (92.3 mg, 242.61 μmol) and tert-butyl ((1R,4R,7R)-2-azabicyclo[2.2.1]heptan-7-yl)carbamate (42.92 mg, 202.17 μmol). The resulting mixture was stirred at RT for 2 h. Upon completion, the mixture was diluted with EA, washed with brine, and dried over anhydrous sodium sulfate. After filtration and evaporation of the solvent in vacuo, the residue was purified by flash column chromatography on silica gel (eluting with 0-8% MeOH in DCM) to give tert-butyl ((1R,4R,7R)-2-(2-(9-(cyclopropylmethyl)-1,2,3,9-tetrahydro-[1,4]oxazino[2,3-g]indol-8-yl)-7-fluoro-1-methyl-1H-benzo[d]imidazole-5-carbonyl)-2-azabicyclo[2.2.1]heptan-7-yl)carbamate (40 mg, 65.07 μmol, 32.1% yield) as a white solid. LC/MS (ESI⁺): m/z 614.7 [(M+H)⁺].

### Step 15:

To a stirred solution of tert-butyl N-[(1R,4R,7R)-2-[2-[9-(cyclopropylmethyl)-2,3-dihydro-1H-pyrrolo[2,3-f][1,4]benzoxazin-8-yl]-7-fluoro-1-methyl-benzimidazole-5-carbonyl]-2-azabicyclo[2.2.1]heptan-7-yl]carbamate (40 mg, 65.07 μmol) in MeOH (2 mL) was added 4 M HCl in dioxane (2 mL). The resulting mixture was stirred at RT for 2 h and monitored by LC/MS. Upon completion, the reaction mixture was basified to pH=8 with saturated Na₂CO₃ solution, then extracted with DCM (30 mL×3). The organic layer was dried over anhydrous sodium sulfate. After filtration and evaporation of the solvent in vacuo, the residue was purified by flash column chromatography on silica gel (eluting with 0-10% MeOH in DCM) to give [(1R,4R,7R)-7-amino-2-azabicyclo[2.2.1]heptan-2-yl]-[2-[9-(cyclopropylmethyl)-2,3-dihydro-1H-pyrrolo[2,3-f][1,4]benzoxazin-8-yl]-7-fluoro-1-methyl-benzimidazol-5-yl]methanone (18 mg, 34.98 μmol, 53.7% yield) as a white solid. LC/MS (ESI⁺): m/z 514.7 [(M+H)⁺]. ¹H NMR (400 MHz, DMSO-d₆) δ 7.58 (d, J=1.2 Hz, 1H), 7.31-7.16 (m, 1H), 7.00 (d, J=8.4 Hz, 1H), 6.91 (s, 1H), 6.65 (d, J=8.4 Hz, 1H), 5.19 (t, J=5.4 Hz, 1H), 4.51 (dd, J=7.1, 3.2 Hz, 2H), 4.15-4.09 (m, 2H), 4.04 (d, J=2.3 Hz, 3H), 3.74 (d, J=2.3 Hz, 1H), 3.51 (dt, J=10.9, 2.9 Hz, 1H), 3.20 (s, 2H), 3.05 (dd, J=17.2, 10.0 Hz, 2H), 2.22 (d, J=3.8 Hz, 1H), 2.05-1.85 (m, 2H), 1.77-1.63 (m, 1H), 1.50-1.39 (m, 1H), 1.39-1.21 (m, 1H), 1.03-0.92 (m, 1H), −0.15 (ddt, J=8.7, 5.6, 2.9 Hz, 2H).

### Example 159 Preparation of [(1R,4R,7R)-7-amino-2-azabicyclo[2.2.1]heptan-2-yl]-[2-[12-(cyclopropylmethyl)-5-methyl-3,4,6,12-tetrazatricyclo[7.3.0.02,6]dodeca-1(9), 2,4,7,10-pentaen-11-yl]-7-fluoro-1-methyl-benzimidazol-5-yl]methanone

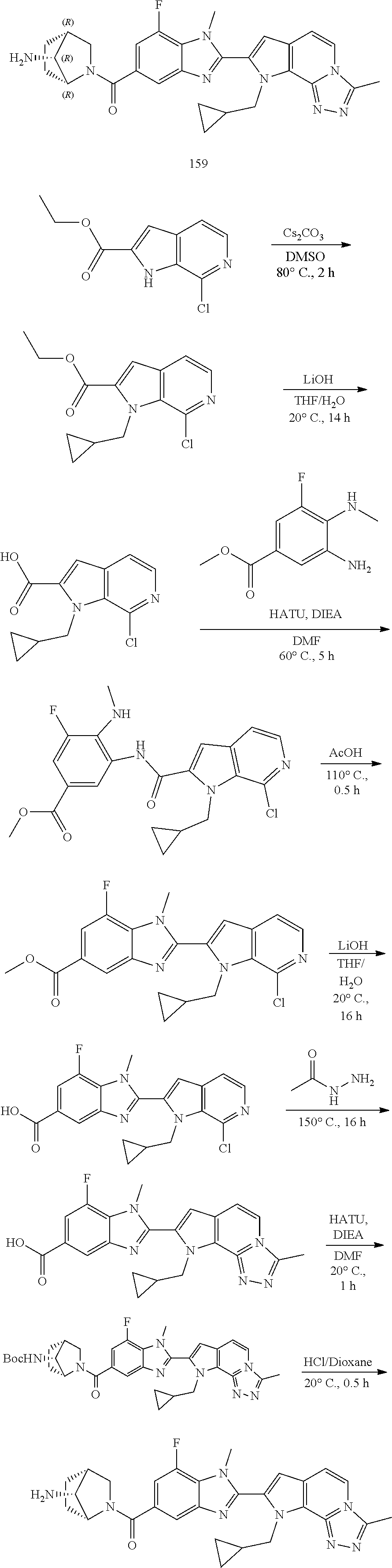

### Step 1:

A mixture of ethyl 7-chloro-1H-pyrrolo[2,3-c]pyridine-2-carboxylate (2.5 g, 11.13 mmol), Cs₂CO₃ (9.06 g, 27.82 mmol) and bromomethyl cyclopropane (2.25 g, 16.69 mmol) in DMSO (40 mL) was stirred at 80° C. for 2 h and monitored by LC/MS. Upon completion, the mixture was diluted with water (200 mL) and extracted with EtOAc (150 mL×2). The combined organic layers were washed with brine, dried over Na₂SO₄, filtered, and concentrated in vacuo. The residue was purified by flash column chromatography on silica gel (eluting with 0-20% EA in PE) to afford ethyl 7-chloro-1-(cyclopropylmethyl)-1H-pyrrolo[2,3-c]pyridine-2-carboxylate (1.82 g, 6.54 mmol, 58.8% yield) as a colorless oil. LC/MS (ESI⁺): m/z 278.7 [(M+H)⁺].

### Step 2:

To a stirred solution of ethyl 7-chloro-1-(cyclopropylmethyl)pyrrolo[2,3-c]pyridine-2-carboxylate (1.92 g, 6.89 mmol) in THF/H₂O mixed solvents (28 mL, 3:1) was added LiOH·H₂O (867.2 mg, 20.66 mmol) and the resulting mixture was stirred at 20° C. for 14 h. Upon completion, the mixture was concentrated in vacuo, diluted with water (20 mL) and acidified with 1 M HCl to PH=5-6. Then the mixture was filtered, and the filter cake was dried in vacuo to afford 7-chloro-1-(cyclopropylmethyl)pyrrolo[2,3-c]pyridine-2-carboxylic acid (1.73 g) as a white solid, which was used in the next step directly without further purification. LC/MS (ESI⁺): m/z 250.8 [(M+H)⁺].

### Step 3:

To a stirred solution of 7-chloro-1-(cyclopropylmethyl)pyrrolo[2,3-c]pyridine-2-carboxylic acid (720 mg, 2.87 mmol) in DMF (12 mL) was successively added HATU (1.42 g, 3.73 mmol), DIEA (1.48 g, 11.49 mmol) and methyl 3-amino-5-fluoro-4-(methylamino)benzoate (683.1 mg, 3.45 mmol). The resulting mixture was stirred at 60° C. for 5 h and monitored by LC/MS. Upon completion, the mixture was diluted with water (60 mL) and extracted with EtOAc (60 mL×2). The combined organic layers were washed with brine, dried over Na₂SO₄, filtered and concentrated in vacuo to afford methyl 3-(7-chloro-1-(cyclopropylmethyl)-1H-pyrrolo[2,3-c]pyridine-2-carboxamido)-5-fluoro-4-(methylamino)benzoate (1.24 g) as a brown oil, which was used in the next step directly without further purification. LC/MS (ESI⁺): m/z 430.7 [(M+H)⁺].

### Step 4:

A mixture of methyl 3-[[7-chloro-1-(cyclopropylmethyl)pyrrolo[2,3-c]pyridine-2-carbonyl]amino]-5-fluoro-4-(methylamino)benzoate (1.24 g, 2.88 mmol) in AcOH (15 mL) was stirred at 110° C. for 0.5 h and monitored by LC/MS. Upon completion, the mixture was concentrated in vacuo. The residue was redissolved in water (50 mL) and EtOAc (50 mL), basified with saturated NaHCO₃ aqueous, and extracted with EtOAc. The combined organic layers were concentrated in vacuo, and the residue was purified by flash column chromatography on silica gel (eluting with 0-20% EA in PE) to afford methyl 2-(7-chloro-1-(cyclopropylmethyl)-1H-pyrrolo[2,3-c]pyridin-2-yl)-7-fluoro-1-methyl-1H-benzo[d]imidazole-5-carboxylate (190 mg, 460.22 μmol, 15.9% yield,) as a yellow solid. LC/MS (ESI⁺): m/z 412.6 [(M+H)⁺].

### Step 5:

To a stirred solution of methyl 2-[7-chloro-1-(cyclopropylmethyl)pyrrolo[2,3-c]pyridin-2-yl]-7-fluoro-1-methyl-benzimidazole-5-carboxylate (220 mg, 532.89 μmol) in THF/H₂O mixed solvents (8 mL, 3:1) was added LiOH·H₂O (67.1 mg, 1.60 mmol). The resulting mixture was stirred at 20° C. for 16 h and monitored by LC/MS. Upon completion, the mixture was concentrated in vacuo, and the residue was redissolved in water (30 mL) and EtOAc (30 mL). The mixture was acidified with HCl (1 M in dioxane) to pH=5 and extracted with EtOAc (30 mL×2). The combined organic layers were dried over Na₂SO₄, filtered and concentrated in vacuo to afford 2-[7-chloro-1-(cyclopropylmethyl)pyrrolo[2,3-c]pyridin-2-yl]-7-fluoro-1-methyl-benzimidazole-5-carboxylic acid (190 mg, 476.41 μmol, 89.4% yield) as a yellow solid. LC/MS (ESI⁺): m/z 398.6 [(M+H)⁺].

### Step 6:

A mixture of 2-[7-chloro-1-(cyclopropylmethyl)pyrrolo[2,3-c]pyridin-2-yl]-7-fluoro-1-methyl-benzimidazole-5-carboxylic acid (200 mg, 501.48 μmol) in acetohydrazide (2 g, 27.00 mmol) was stirred at 150° C. for 16 h and monitored by LC/MS. Upon completion, the mixture was cooled to 20° C., and then purified by reversed phase column to afford 2-(9-(cyclopropylmethyl)-3-methyl-9H-pyrrolo[2,3-c][1,2,4]triazolo[4,3-a]pyridin-8-yl)-7-fluoro-1-methyl-1H-benzo[d]imidazole-5-carboxylic acid (19 mg, 45.41 μmol, 9.0% yield) as a white solid. LC/MS (ESI⁺): m/z 419.1 [(M+H)⁺].

### Step 7:

To a stirred solution of 2-[12-(cyclopropylmethyl)-5-methyl-3,4,6,12-tetrazatricyclo[7.3.0.0^{2,6}]dodeca-1(9), 2,4,7,10-pentaen-11-yl]-7-fluoro-1-methyl-benzimidazole-5-carboxylic acid (20 mg, 47.80 μmol) in DMF (3 mL) was successively added HATU (23.6 mg, 62.14 μmol), DIEA (24.7 mg, 191.19 μmol) and tert-butyl ((1R,4R,7R)-2-azabicyclo[2.2.1]heptan-7-yl)carbamate (12.2 mg, 57.36 μmol). The resulting mixture was stirred at 20° C. for 1 h and monitored by LC/MS. Upon completion, the mixture was diluted with water (20 mL) and extracted with EtOAc (20 mL×2). The combined organic layers were washed with brine, dried over Na₂SO₄, filtered, and concentrated in vacuo. The residue was purified by prep-TLC to afford tert-butyl ((1R,4R,7R)-2-(2-(9-(cyclopropylmethyl)-3-methyl-9H-pyrrolo[2,3-c][1,2,4]triazolo[4,3-a]pyridin-8-yl)-7-fluoro-1-methyl-1H-benzo[d]imidazole-5-carbonyl)-2-azabicyclo[2.2.1]heptan-7-yl)carbamate (18 mg, 29.38 μmol, 61.4% yield) as a white solid. LC/MS (ESI⁺): m/z 612.7 [(M+H)⁺].

### Step 8:

To a stirred solution of tert-butyl N-[(1R,4R,7R)-2-[2-[12-(cyclopropylmethyl)-5-methyl-3,4,6,12-tetrazatricyclo[7.3.0.0^{2,6}]dodeca-1(9), 2,4,7,10-pentaen-11-yl]-7-fluoro-1-methyl-benzimidazole-5-carbonyl]-2-azabicyclo[2.2.1]heptan-7-yl]carbamate (20 mg, 32.64 μmol) in MeOH (1 mL) was added 4 M HCl in dioxane (2 mL). The mixture was stirred at 20° C. for 0.5 h and monitored by LC/MS. Upon completion, the mixture was concentrated in vacuo, and the residue was purified by prep-HPLC to afford [(1R,4R,7R)-7-amino-2-azabicyclo[2.2.1]heptan-2-yl]-[2-[12-(cyclopropylmethyl)-5-methyl-3,4,6,12-tetrazatricyclo[7.3.0.0^{2,6}]dodeca-1(9), 2,4,7,10-pentaen-11-yl]-7-fluoro-1-methyl-benzimidazol-5-yl]methanone (8.5 mg, 16.49 μmol, 50.5% yield) as a white solid. LC/MS (ESI⁺): m/z 512.7 [(M+H)⁺]. ¹H NMR (400 MHz, DMSO-d₆) δ 7.99 (d, J=7.2 Hz, 1H), 7.76-7.61 (m, 1H), 7.34-7.21 (m, 3H), 4.99 (d, J=7.1 Hz, 2H), 4.09 (s, 3H), 3.72 (s, 1H), 3.51 (dt, J=11.4, 3.1 Hz, 1H), 3.20 (s, 1H), 3.11-2.99 (m, 1H), 2.72 (s, 3H), 2.25-2.08 (m, 1H), 2.03-1.82 (m, 2H), 1.78-1.61 (m, 1H), 1.48-1.32 (m, 1H), 1.28-1.17 (m, 1H), 0.35-0.14 (m, 4H).

### Example 160 Synthesis of 8-(5-((1R,4R,7R)-7-amino-2-azabicyclo[2.2.1]heptane-2-carbonyl)-7-fluoro-1-methyl-1H-benzo[d]imidazol-2-yl)-9-(cyclopropylmethyl)-2,9-dihydro-3H-pyrrolo[2,3-c][1,2,4]triazolo[4,3-a]pyridin-3-one

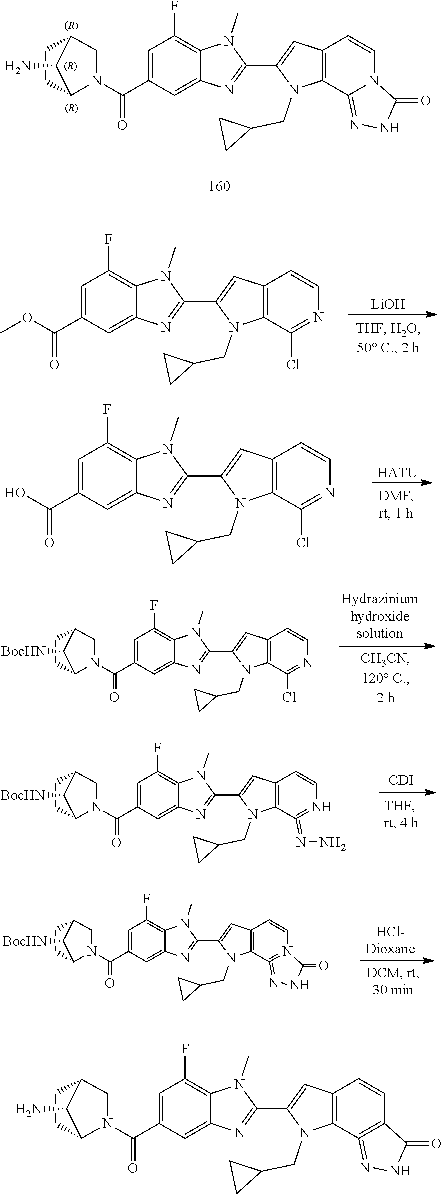

### Step 1:

A mixture of methyl 2-[7-chloro-1-(cyclopropylmethyl)pyrrolo[2,3-c]pyridin-2-yl]-7-fluoro-1-methyl-benzimidazole-5-carboxylate (60.0 mg, 145.33 μmol, intermediate of example 159), LiOH·H₂O (30.5 mg, 726.67 μmol) in H₂O/THF mixed solvents (4 mL, 1:1). The resulting mixture was stirred at 50° C. for 2 h and monitored by LC/MS. Upon completion, the mixture was acidified with 3 M hydrochloric acid. The mixture was diluted with EtOAc (50 ml) and washed with water (25 ml). The organic layer was dried over sodium sulfate, filtered, and concentrated in vacuo. The crude material was purified by flash column chromatography on silica gel (eluting with 0-100% EA in PE) to give 2-[7-chloro-1-(cyclopropylmethyl)pyrrolo[2,3-c]pyridin-2-yl]-7-fluoro-1-methyl-benzimidazole-5-carboxylic acid (50.0 mg, 125.37 μmol, 86.2% yield) as a yellow oil. LC/MS (ESI⁺): m/z 398.8 [(M+H)⁺].

### Step 2:

A mixture of tert-butyl N-[(1R,4R,7R)-2-azabicyclo[2.2.1]heptan-7-yl]carbamate (31.9 mg, 150.44 μmol), 2-[7-chloro-1-(cyclopropylmethyl)pyrrolo[2,3-c]pyridin-2-yl]-7-fluoro-1-methyl-benzimidazole-5-carboxylic acid (50.0 mg, 125.37 μmol), HATU (71.5 mg, 188.06 μmol) and DIPEA (32.4 mg, 250.74 μmol) in DMF (2 mL) was stirred at RT for 1 h and monitored by LC/MS. Upon completion, the reaction mixture was diluted with EtOAc (50 ml) and washed with water (25 ml). The organic layer was dried over sodium sulfate, filtered, and concentrated in vacuo. The crude material was purified by flash column chromatography on silica gel (eluting with 0-100% EA in PE) to give tert-butyl N-[(1R,4R,7R)-2-[2-[7-chloro-1-(cyclopropylmethyl)pyrrolo[2,3-c]pyridin-2-yl]-7-fluoro-1-methyl-benzimidazole-5-carbonyl]-2-azabicyclo[2.2.1]heptan-7-yl]carbamate (50.0 mg, 84.30 μmol, 67.2% yield) as a yellow solid. LC/MS (ESI⁺): m/z 592.8 [(M+H)⁺].

### Step 3:

A mixture of tert-butyl N-[(1R,4R,7R)-2-[2-[7-chloro-1-(cyclopropylmethyl)pyrrolo[2,3-c]pyridin-2-yl]-7-fluoro-1-methyl-benzimidazole-5-carbonyl]-2-azabicyclo[2.2.1]heptan-7-yl]carbamate (50.0 mg, 84.30 μmol), hydrazine hydrate (21.1 mg, 421.52 μmol) in acetonitrile (2 mL) was stirred at 120° C. for 2 h and monitored by LC/MS. Upon completion, the mixture was concentrated in vacuo and purified by prep-HPLC to afford tert-butyl N-[(1R,4R,7R)-2-[2-[(7Z)-1-(cyclopropylmethyl)-7-hydrazinylidene-6H-pyrrolo[2,3-c]pyridin-2-yl]-7-fluoro-1-methyl-benzimidazole-5-carbonyl]-2-azabicyclo[2.2.1]heptan-7-yl]carbamate (20 mg, 33.97 μmol, 40.3% yield) as a white solid. LC/MS (ESI⁺): m/z 588.8 [(M+H)⁺].

### Step 4:

A mixture of tert-butyl N-[(1R,4R,7R)-2-[2-[(7Z)-1-(cyclopropylmethyl)-7-hydrazinylidene-6H-pyrrolo[2,3-c]pyridin-2-yl]-7-fluoro-1-methyl-benzimidazole-5-carbonyl]-2-azabicyclo[2.2.1]heptan-7-yl]carbamate (20.0 mg, 33.97 μmol) and di(imidazol-1-yl)methanone (5.5 mg, 33.97 μmol) in THF (4 mL) was stirred at RT for 4 h and monitored by LC/MS. Upon completion, the reaction mixture was diluted with EtOAc (50 ml) and washed with water (25 ml). The organic layer was dried over sodium sulfate, filtered, and concentrated in vacuo. The crude material was purified by flash column chromatography on silica gel (eluting with 0-100% EA in PE) to give tert-butyl N-[(1R,4R,7R)-2-[2-[12-(cyclopropylmethyl)-5-oxo-3,4,6,12-tetrazatricyclo[7.3.0.02,6]dodeca-1(9), 2,7,10-tetraen-11-yl]-7-fluoro-1-methyl-benzimidazole-5-carbonyl]-2-azabicyclo[2.2.1]heptan-7-yl]carbamate (4 mg, 6.51 μmol, 19.1% yield) as a yellow solid. LC/MS (ESI⁺): m/z 614.8 [(M+H)⁺].

### Step 5:

To a solution of tert-butyl N-[(1R,4R,7R)-2-[2-[12-(cyclopropylmethyl)-5-oxo-3,4,6,12-tetrazatricyclo[7.3.0.02,6]dodeca-1(9), 2,7,10-tetraen-11-yl]-7-fluoro-1-methyl-benzimidazole-5-carbonyl]-2-azabicyclo[2.2.1]heptan-7-yl]carbamate (4.0 mg, 6.51 μmol) in dioxane was added 4 M HCl in dioxane (3 mL). The resulting mixture was stirred at RT for 30 min and monitored by LC/MS. Upon completion, solvent was removed in vacuo and the residue was purified by prep-HPLC to afford 11-[5-[(1R,4R,7R)-7-amino-2-azabicyclo[2.2.1]heptane-2-carbonyl]-7-fluoro-1-methyl-benzimidazol-2-yl]-12-(cyclopropylmethyl)-3,4,6,12-tetrazatricyclo[7.3.0.02,6]dodeca-1(9), 2,7,10-tetraen-5-one (0.5 mg, 9.72e-1 μmol, 14.9% yield) as a white solid. LC/MS (ESI⁺): m/z 514.8 [(M+H)⁺].

### Example 161 Preparation of ((1R,4R,7R)-7-amino-2-azabicyclo[2.2.1]heptan-2-yl)(2-(9-(cyclopropylmethyl)-1,2,3,9-tetrahydro-[1,4]thiazino[2,3-g]indol-8-yl)-7-fluoro-1-methyl-1H-benzo[d]imidazol-5-yl)methanone

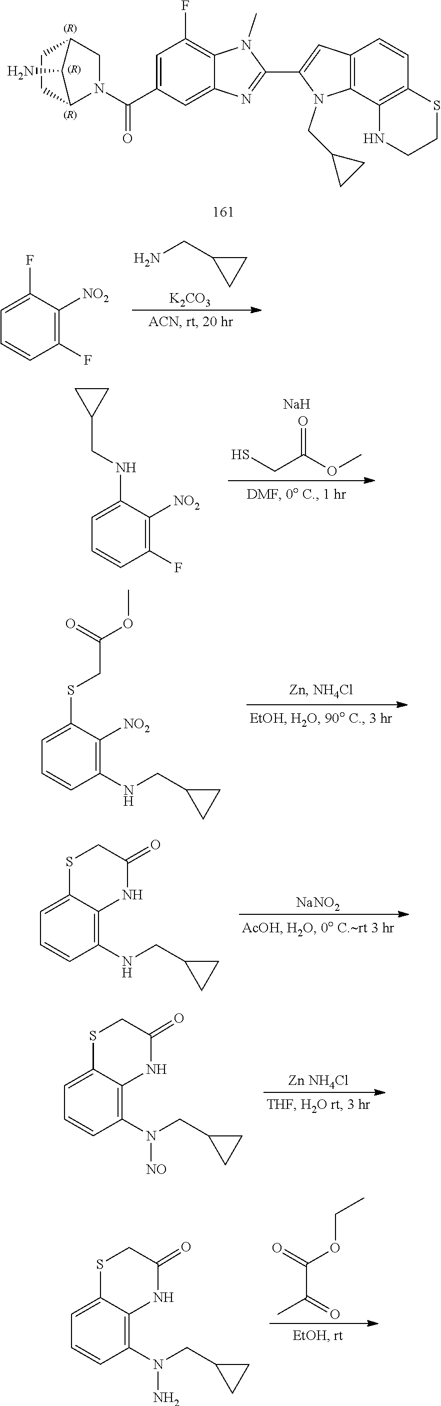

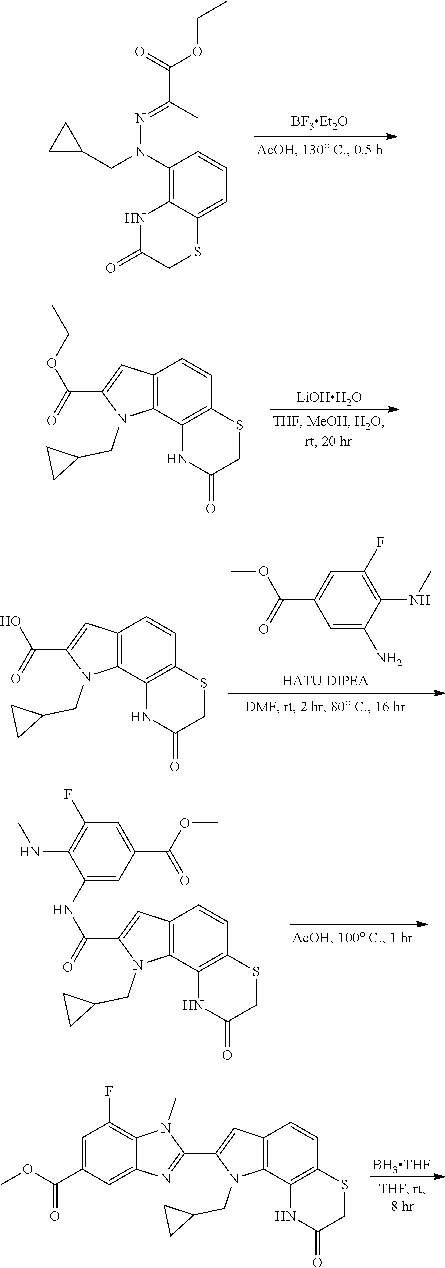

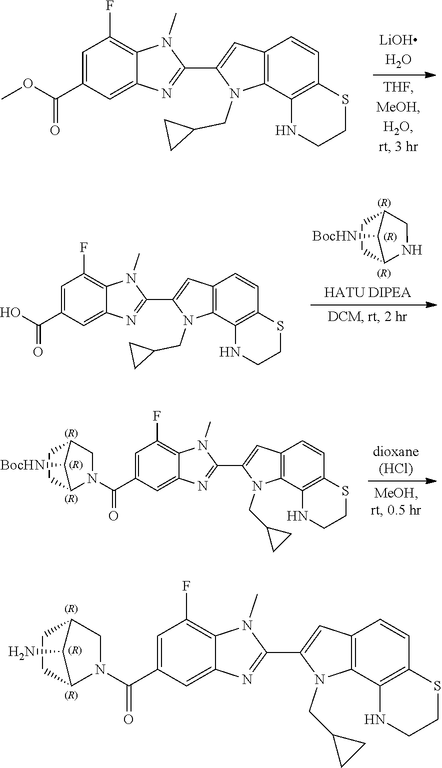

### Step 1:

To a stirred mixture of 1,3-difluoro-2-nitrobenzene (5.0 g, 31.43 mmol) and potassium carbonate (8.69 g, 62.86 mmol) in acetonitrile (500 mL) was added cyclopropylmethanamine (2.24 g, 31.43 mmol) and the resulting mixture was stirred under nitrogen atmosphere at RT for 20 h. Upon completion, the mixture was filtered and concentrated in vacuo. The residue was purified by flash column chromatography on silica gel (eluting with 1-10% EA in PE) to afford N-(cyclopropylmethyl)-3-fluoro-2-nitro-aniline (6.1 g, 29.02 mmol, 92.3% yield) as an orange red liquid. LC/MS (ESI⁺): m/z 210.8 [(M+H)⁺].

### Step 2:

To a stirred mixture of sodium hydride (60% dispersion in mineral oil, 800.59 mg, 34.82 mmol) in DMF (90 mL) at 0° C. was added methyl 2-sulfanylacetate (3.39 g, 31.92 mmol) dropwise over 15 min. The reaction mixture was stirred at 0° C. for 0.5 h. Then a solution of N-(cyclopropylmethyl)-3-fluoro-2-nitro-aniline (6.1 g, 29.02 mmol) in DMF (12 mL) was added dropwise and stirring was continued for 1 h at 0° C. Upon completion, the reaction was quenched with H₂O (200 mL) and extracted with EtOAc (3×500 mL). The combined organic layers were washed with brine (500 mL), dried over anhydrous Na₂SO₄, filtered, and concentrated in vacuo. The crude product was purified by flash column chromatography on silica gel (eluting with 1-60% EA in PE) to afford desired product methyl 2-[3-(cyclopropylmethylamino)-2-nitro-phenyl]sulfanylacetate (7.4 g, 24.97 mmol, 86.0% yield) as an orange red solid. LC/MS (ESI⁺): m/z 296.8 [(M+H)⁺].

### Step 3:

To a stirred solution of methyl 2-[3-(cyclopropylmethylamino)-2-nitro-phenyl]sulfanylacetate (7.4 g, 24.97 mmol) ammonium chloride (9.35 g, 174.80 mmol) in EtOH/water mixed solvents (180 mL, 5:4) was added zinc powder (9.80 g, 149.83 mmol) in portions. The mixture was stirred at 90° C. for 3 h. Upon completion, the reaction mixture was cooled down, filtered through a pad of Celite, and the filter cake was washed with EtOH (3×30 mL). The filtrate was concentrated in vacuo and the residue was extracted with EA (3×100 mL). The combined organic layers were washed with water (100 mL) and bine (100 mL), dried over sodium sulfate, and concentrated in vacuo. The crude product was purified by flash column chromatography on silica gel (eluting with 0-60% EA in PE) to obtain 5-(cyclopropylmethylamino)-4H-1,4-benzothiazin-3-one (3.5 g, 14.94 mmol, 59.8% yield) as a brown white solid. LC/MS (ESI⁺): m/z 234.8 [(M+H)⁺].

### Step 4:

To a stirred mixture of 5-(cyclopropylmethylamino)-4H-1,4-benzothiazin-3-one (3.15 g, 13.44 mmol) in water/AcOH mixed solvents (22 mL, 1:2) was added a solution of sodium nitrite (973.97 mg, 14.12 mmol) in water (3 mL) dropwise at 0° C. Precipitation was formed and the reaction mixture was stirred for 3 h. The mixture was filtered, and the filtrate was extracted with CH₂Cl₂ (2×50 mL). Combined organic extracts were washed with brine (20 mL), dried over sodium sulfate, and concentrated in vacuo. The residue and the previous filter cake together give the crude product N-(cyclopropylmethyl)-N-(3-oxo-4H-1,4-benzothiazin-5-yl)nitrous amide (3.5 g, crude) as a brown solid. LC/MS (ESI⁺): m/z 263.8 [(M+H)⁺].

### Step 5:

To a stirred mixture of N-(cyclopropylmethyl)-N-(3-oxo-4H-1,4-benzothiazin-5-yl)nitrous amide (3.5 g, 13.29 mmol) and ammonium chloride (4.27 g, 79.75 mmol) in water/THF mixed solvents (50 mL, 2:3) was added zinc powder (4.35 g, 66.46 mmol) in portions at 0° C. The reaction mixture was stirred with ice-water bath for 1 h, then warmed to RT and stirred for another 2 h. The reaction mixture was filtered, and the filter cake was washed with THF. The filtrate was extracted with EtOAc (2×50 mL) and the organic layers were concentrated in vacuo. The residue was purified by flash column chromatography on silica gel (eluting with 0-60% EA in PE) to give 5-[amino(cyclopropylmethyl)amino]-4H-1,4-benzothiazin-3-one (1.4 g, 5.62 mmol, 42.2% yield) as a grey solid. LC/MS (ESI⁺): m/z 249.8 [(M+H)⁺].

### Step 6:

A mixture of 5-[amino(cyclopropylmethyl)amino]-4H-1,4-benzothiazin-3-one (1.4 g, 5.62 mmol) and ethyl 2-oxopropanoate (684.59 mg, 5.90 mmol) in ethanol (15 mL) was stirred at RT for 2 h. Upon completion, the reaction mixture was concentrated in vacuo to give ethyl (2E)-2-[cyclopropylmethyl-(3-oxo-4H-1,4-benzothiazin-5-yl)hydrazono]propanoate (1.94 g), which was used in the next step without further purification. LC/MS (ESI⁺): m/z 347.8 [(M+H)⁺].

### Step 7:

To a stirred solution of ethyl (2E)-2-[cyclopropylmethyl-(3-oxo-4H-1,4-benzothiazin-5-yl)hydrazono]propanoate (1.94 g, 5.58 mmol) in acetic acid (10 mL) was added boron trifluoride diethyl etherate (792.51 mg, 5.58 mmol). The mixture was stirred at 130° C. for 0.5 h. Upon completion, the reaction mixture was cooled down to RT, then quenched by saturated aqueous solution of NaHCO₃ (100 mL). The mixture was extracted with EA (3×60 mL) and the combined organic layers were washed with water (50 mL) and brine (50 mL), dried over sodium sulfate, and concentrated in vacuo. The residue was purified by flash column chromatography on silica gel (eluting with 0-30% EA in PE) to afford ethyl 9-(cyclopropylmethyl)-2-oxo-1H-pyrrolo[2,3-f][1,4]benzothiazine-8-carboxylate (0.32 g, 968.52 μmol, 17.3% yield) as a brown solid. LC/MS (ESI⁺): m/z 330.8 [(M+H)⁺].

### Step 8:

To a stirred solution of ethyl 9-(cyclopropylmethyl)-2-oxo-1H-pyrrolo[2,3-f][1,4]benzothiazine-8-carboxylate (320 mg, 968.52 μmol) in THF/MeOH mixed solvents (5 mL, 3:2) was added a solution of lithium hydroxide monohydrate (203.20 mg, 4.84 mmol) in water (1 mL) and the resulting mixture was stirred at RT for 20 h. The reaction mixture was concentrated in vacuo, then taken up in water (5 mL), acidified with 2 M aqueous HCl and extracted with MeOH/CH₂Cl₂ (2×20 mL). Combined organic layers were dried over anhydrous sodium sulphate, filtered, and concentrated in vacuo to afford the desired product 9-(cyclopropylmethyl)-2-oxo-1H-pyrrolo[2,3-f][1,4]benzothiazine-8-carboxylic acid (240 mg, 793.79 μmol, 81.9% yield) as an off-white solid. LC/MS (ESI⁺): m/z 302.8 [(M+H)⁺].

### Step 9:

To a stirred solution of 9-(cyclopropylmethyl)-2-oxo-1H-pyrrolo[2,3-f][1,4]benzothiazine-8-carboxylic acid (240 mg, 793.79 μmol) and methyl 3-amino-5-fluoro-4-(methylamino)benzoate (173.06 mg, 873.17 μmol) in DMF (5 mL) at RT were added HATU (392.37 mg, 1.03 mmol) and DIPEA (307.77 mg, 2.38 mmol). The mixture was stirred at RT for 2 h, then heated to 80° C. for 16 h. Upon completion, the reaction was quenched with H₂O (15 mL) and extracted with CH₂Cl₂ (2×30 mL). Combined organic extracts were washed with brine (30 mL), dried over sodium sulfate, and concentrated in vacuo. The residue was purified by flash column chromatography on silica gel (eluting with 2-80% EA in PE) to afford methyl 3-[[9-(cyclopropylmethyl)-2-oxo-1H-pyrrolo[2,3-f][1,4]benzothiazine-8-carbonyl]amino]-5-fluoro-4-(methylamino)benzoate (200 mg, 414.49 μmol, 52.2% yield) as a grey solid. LC/MS (ESI⁺): m/z 482.7 [(M+H)⁺].

### Step 10:

A mixture of methyl 3-[[9-(cyclopropylmethyl)-2-oxo-1H-pyrrolo[2,3-f][1,4]benzothiazine-8-carbonyl]amino]-5-fluoro-4-(methylamino)benzoate (200 mg, 414.49 μmol) in acetic acid (3 mL) was stirred at 100° C. for 1.0 h under an atmosphere of N₂. Upon completion, the reaction mixture was concentrated in vacuo, then diluted with EtOAc (10 mL), basified with saturated NaHCO₃, and extracted with EtOAc (2×15 mL). Combined organic extracts were washed with brine (20 mL), dried over sodium sulfate, and concentrated in vacuo. The residue was purified by flash column chromatography on silica gel (eluting with 1-20% MeOH in CH₂Cl₂) to afford the title product methyl 2-[9-(cyclopropylmethyl)-2-oxo-1H-pyrrolo[2,3-f][1,4]benzothiazin-8-yl]-7-fluoro-1-methyl-benzimidazole-5-carboxylate (140 mg, 301.39 μmol, 72.7% yield) as a white solid. LC/MS (ESI⁺): m/z 464.7 [(M+H)⁺].

### Step 11:

To a suspension of methyl 2-[9-(cyclopropylmethyl)-2-oxo-1H-pyrrolo[2,3-f][1,4]benzothiazin-8-yl]-7-fluoro-1-methyl-benzimidazole-5-carboxylate (140 mg, 301.39 μmol) in THF (1 mL) was added 1.0 M borane-tetrahydrofuran complex (0.1 mL) at RT and the resulting mixture was stirred for 7 h under N₂ atmosphere. Upon completion, the reaction was quenched with ice-cold water (10 mL) and extracted with EtOAc (3×10 mL). The combined organic layers were washed with brine (20 mL), dried over anhydrous Na₂SO₄, filtered, and concentrated in vacuo. The residue was purified by flash chromatography on silica gel (eluting with 0-20% MeOH in CH₂Cl₂) to afford methyl 2-[9-(cyclopropylmethyl)-2,3-dihydro-1H-pyrrolo[2,3-f][1,4]benzothiazin-8-yl]-7-fluoro-1-methyl-benzimidazole-5-carboxylate (35 mg, 77.69 μmol, 25.7% yield) as a solid. LC/MS (ESI⁺): m/z 450.7 [(M+H)⁺].

### Step 12:

To a stirred mixture of methyl 2-[9-(cyclopropylmethyl)-2,3-dihydro-1H-pyrrolo[2,3-f][1,4]benzothiazin-8-yl]-7-fluoro-1-methyl-benzimidazole-5-carboxylate (35 mg, 77.69 μmol) in THF/MeOH mixed solvents (1.5 mL, 2:1) was added a solution of lithium hydroxide monohydrate (16.30 mg, 388.43 μmol) in water (0.5 mL) and the resulting mixture was stirred at RT for 3 h. Upon completion, the reaction mixture was concentrated in vacuo, then taken up in water (5 mL), acidified with 2 M aqueous hydrochloric acid and extracted with CH₂Cl₂ (2×10 mL). Combined organic layers were dried over anhydrous sodium sulphate, filtered, and concentrated in vacuo to afford 2-[9-(cyclopropylmethyl)-2,3-dihydro-1H-pyrrolo[2,3-f][1,4]benzothiazin-8-yl]-7-fluoro-1-methyl-benzimidazole-5-carboxylic acid (33 mg, 75.60 μmol, 97.3% yield) as an off-white solid. LC/MS (ESI⁺): m/z 436.8 [(M+H)⁺].

### Step 13:

To a stirred solution of 2-[9-(cyclopropylmethyl)-2,3-dihydro-1H-pyrrolo[2,3-f][1,4]benzothiazin-8-yl]-7-fluoro-1-methyl-benzimidazole-5-carboxylic acid (34 mg, 77.89 μmol), tert-butyl N-[(1R,4R,7R)-2-azabicyclo[zh2.2.1]heptan-7-yl]carbamate (16.54 mg, 77.89 μmol) in CH₂Cl₂ (3.0 mL) at RT were added HATU (38.50 mg, 101.26 μmol) and DIPEA (30.20 mg, 233.68 μmol). The resulting mixture was stirred at RT for 2 h. Upon completion, solvent was removed in vacuo. The residue was purified by flash column chromatography on silica gel (eluting with 1-20% MeOH in CH₂Cl₂) to afford tert-butyl N-[(1R,4R,7R)-2-[2-[9-(cyclopropylmethyl)-2,3-dihydro-1H-pyrrolo[2,3-f][1,4]benzothiazin-8-yl]-7-fluoro-1-methyl-benzimidazole-5-carbonyl]-2-azabicyclo[2.2.1]heptan-7-yl]carbamate (40 mg, 63.41 μmol, 81.4% yield) as a white solid. LC/MS (ESI⁺): m/z 630.7 [(M+H)⁺].

### Step 14:

To a stirred solution tert-butyl N-[(1R,4R,7R)-2-[2-[9-(cyclopropylmethyl)-2,3-dihydro-1H-pyrrolo[2,3-f][1,4]benzothiazin-8-yl]-7-fluoro-1-methyl-benzimidazole-5-carbonyl]-2-azabicyclo[2.2.1]heptan-7-yl]carbamate (20 mg, 31.71 μmol) in MeOH (0.5 mL) was added 4 M HCl in dioxane (2 mL) and the resulting mixture was stirred at RT for 0.5 h. Upon completion, solvent was removed in vacuo. The residue was purified by prep-HPLC to give [(1R,4R,7R)-7-amino-2-azabicyclo[2.2.1]heptan-2-yl]-[2-[9-(cyclopropylmethyl)-2,3-dihydro-1H-pyrrolo[2,3-f][1,4]benzothiazin-8-yl]-7-fluoro-1-methyl-benzimidazol-5-yl]methanone (4.2 mg, 7.91 μmol, 24.9% yield) as a white solid. LC/MS (ESI⁺): m/z 530.7 [(M+H)⁺]. ¹H NMR (400 MHz, DMSO-d₆) δ 7.64 (d, J=42.2 Hz, 1H), 7.25 (dd, J=30.2, 11.8 Hz, 1H), 7.01 (d, J=8.2 Hz, 1H), 6.92 (s, 1H), 6.72 (d, J=8.2 Hz, 1H), 5.40 (s, 1H), 4.46 (d, J=6.6 Hz, 2H), 4.04 (s, 3H), 3.72 (s, 1H), 3.60 (s, 2H), 3.50 (d, J=10.8 Hz, 1H), 3.19 (s, 1H), 3.07 (d, J=11.0 Hz, 1H), 3.01 (s, 1H), 2.21 (s, 1H), 1.95 (s, 2H), 1.77-1.64 (m, 1H), 1.49-1.35 (m, 1H), 0.93 (s, 1H), 0.17 (d, J=7.8 Hz, 2H), −0.16 (d, J=5.0 Hz, 2H).

### Example 162 Preparation of ((1R,4R,7R)-7-amino-2-azabicyclo[2.2.1]heptan-2-yl)(2-(9-(cyclopropylmethyl)-4-oxido-1,2,3,9-tetrahydro-[1,4]thiazino[2,3-g]indol-8-yl)-7-fluoro-1-methyl-1H-benzo[d]imidazol-5-yl)methanone

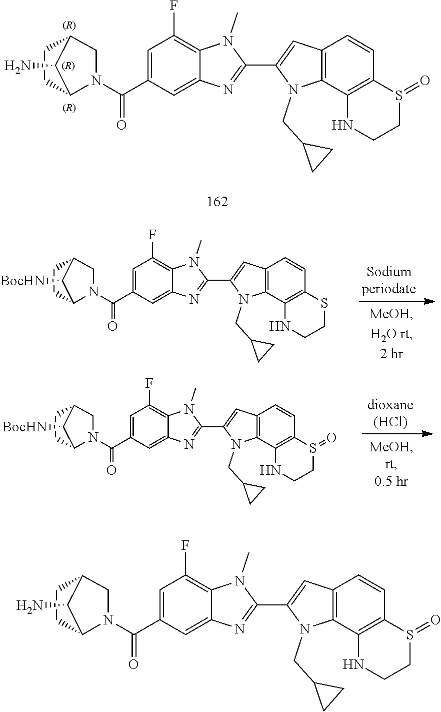

### Step 1:

To a stirred mixture of tert-butyl N-[(1R,4R,7R)-2-[2-[9-(cyclopropylmethyl)-2,3-dihydro-1H-pyrrolo[2,3-f][1,4]benzothiazin-8-yl]-7-fluoro-1-methyl-benzimidazole-5-carbonyl]-2-azabicyclo[2.2.1]heptan-7-yl]carbamate (20 mg, 31.71 μmol, intermediate of example 161) in MeOH/H₂O mixed solvents (1.4 mL, 5:2) was added sodium periodate (20.35 mg, 95.12 μmol). The mixture was stirred at RT for 2 h, then concentrated in vacuo. The residue was purified by flash column chromatography on silica gel (eluting with 0-20% MeOH in CH₂Cl₂) to give tert-butyl N-[(1R,4R,7R)-2-[2-[9-(cyclopropylmethyl)-4-oxo-2,3-dihydro-1H-pyrrolo[2,3-f][1,4]benzothiazin-8-yl]-7-fluoro-1-methyl-benzimidazole-5-carbonyl]-2-azabicyclo[2.2.1]heptan-7-yl]carbamate (12 mg, 18.55 μmol, 58.5% yield) as a white solid. LC/MS (ESI⁺): m/z 646.7 [(M+H)⁺].

### Step 2:

To a stirred solution tert-butyl N-[(1R,4R,7R)-2-[2-[9-(cyclopropylmethyl)-4-oxo-2,3-dihydro-1H-pyrrolo[2,3-f][1,4]benzothiazin-8-yl]-7-fluoro-1-methyl-benzimidazole-5-carbonyl]-2-azabicyclo[2.2.1]heptan-7-yl]carbamate (12 mg, 18.55 μmol) in MeOH (0.5 mL) was added 4 M HCl in dioxane (2 mL) and the resulting mixture was stirred at RT for 0.5 h. The reaction mixture was evaporated to afford the crude product, which was purified by prep-HPLC to give [(1R,4R,7R)-7-amino-2-azabicyclo[2.2.1]heptan-2-yl]-[2-[9-(cyclopropylmethyl)-4-oxo-2,3-dihydro-1H-pyrrolo[2,3-f][1,4]benzothiazin-8-yl]-7-fluoro-1-methyl-benzimidazol-5-yl]methanone (5 mg, 8.44 μmol, 45.4% yield) as a white solid. LC/MS (ESI⁺): m/z 564.8 [(M+H)⁺]. ¹H NMR (400 MHz, DMSO-d₆) δ 7.98 (s, 0.4H), 7.45 (d, J=43.2 Hz, 1H), 7.06 (dd, J=30.8, 11.8 Hz, 1H), 6.73 (s, 1H), 6.62 (s, 1H), 5.33 (d, J=4.8 Hz, 1H), 4.26 (d, J=6.8 Hz, 2H), 3.84 (s, 3H), 3.53 (s, 1H), 3.39 (s, 2H), 3.30 (d, J=10.6 Hz, 1H), 3.00 (s, 1H), 2.87 (d, J=11.0 Hz, 1H), 2.81-2.77 (m, 1H), 1.99 (d, J=29.0 Hz, 1H), 1.80-1.63 (m, 2H), 1.51 (dd, J=17.8, 10.7 Hz, 1H), 1.21 (dd, J=21.1, 9.8 Hz, 1H), 0.81-0.68 (m, 1H), −0.01 (d, J=8.0 Hz, 2H), −0.35 (d, J=5.0 Hz, 2H).

### Example 163 Synthesis of ((1R,4R,7R)-7-amino-2-azabicyclo[2.2.1]heptan-2-yl)(2-(1-(cyclopropylmethyl)-6,7,8,9-tetrahydro-1H-pyrrolo[2,3-f]quinolin-2-yl)-7-fluoro-1-methyl-1H-benzo[d]imidazol-5-yl)methanone

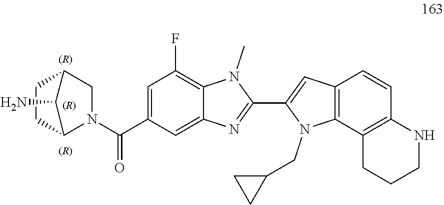

Prepared in analogous manner as for Example 151. LC/MS (ESI⁺): m/z 513.2 [(M+H)⁺].

### Example 164 Preparation of ((1R,4R,7R)-7-amino-2-azabicyclo[2.2.1]heptan-2-yl)(2-(3-cyclobutyl-2,3-dihydro-1H-pyrrolo[1,2,3-de]quinoxalin-5-yl)-7-fluoro-1-methyl-1H-benzo[d]imidazol-5-yl)methanone

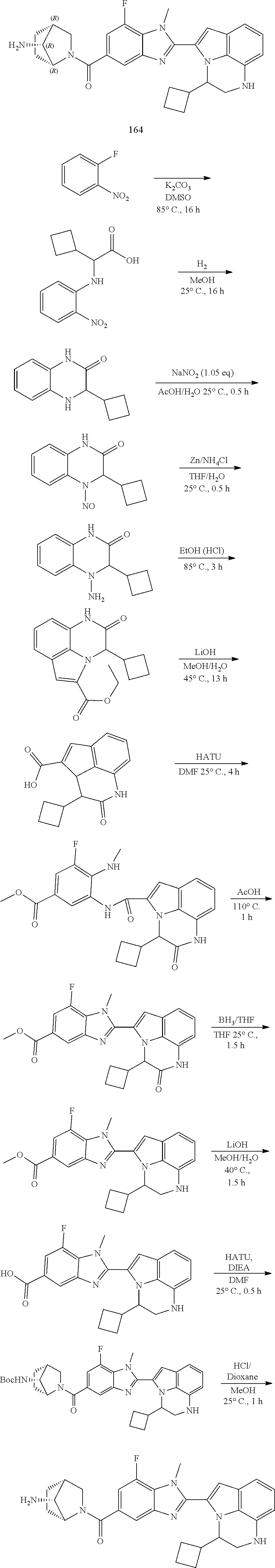

### Step 1:

To a mixture of 1-fluoro-2-nitrobenzene (4.6 g, 32.60 mmol) in DMSO (100 mL) were added K₂CO₃ (13.52 g, 97.80 mmol) and amino(cyclobutyl)acetic acid (4.63 g, 35.86 mmol). The mixture was stirred at 85° C. for 16 h under N₂ atmosphere. Upon completion indicated by LC/MS, the mixture was diluted with water (500 mL) and extracted with TBME (200 mL). The aqueous layer was acidified with 1 M HCl to pH=4-5, and then extracted with EtOAc (300 mL×2). The combined organic layers were dried over Na₂SO₄, filtered and concentrated in vacuo to afford 2-cyclobutyl-2-((2-nitrophenyl)amino)acetic acid (7.8 g) as a yellow oil, which was used in the next step directly without further purification. LC/MS (ESI⁺): m/z 251.1 [(M+H)⁺].

### Step 2:

A mixture of 2-cyclobutyl-2-(2-nitroanilino)acetic acid (7.8 g, 31.17 mmol) and 10 wt % Pd/C (1 g, 9.40 mmol) in MeOH (200 mL) under H₂ (15 psi) atmosphere was stirred at 25° C. for 16 h. Upon completion, the mixture was filtered, and the filtrate was concentrated in vacuo. The residue was purified by flash column chromatography on silica gel (eluting with 0-25% EA in PE) to afford 3-cyclobutyl-3,4-dihydroquinoxalin-2(1H)-one (5.85 g, 26.03 mmol, 83.5% yield) as a yellow solid. LC/MS (ESI⁺): m/z 203.1 [(M+H)⁺].

### Step 3:

To a mixture of 3-cyclobutyl-3,4-dihydro-1H-quinoxalin-2-one (5.85 g, 28.92 mmol) in AcOH (70 mL) was dropwise added a solution of NaNO₂ (2.20 g, 31.82 mmol) in water (35 mL) and the resulting mixture was stirred at 25° C. for 0.5 h. Upon completion, the mixture was filtered, and the filtered cake was concentrated in vacuo to afford 3-cyclobutyl-4-nitroso-3,4-dihydroquinoxalin-2(1H)-one (5 g) as a white solid, which was used in the next step directly without further purification.

### Step 4:

To a stirred mixture of 3-cyclobutyl-4-nitroso-1,3-dihydroquinoxalin-2-one (5 g, 21.62 mmol) and NH₄Cl (7.75 g, 144.86 mmol) in THF/H₂O (100 mL, 1:1) mixed solvents was added zinc powder (5.66 g, 86.49 mmol) in portions. The mixture was stirred at 25° C. for 0.5 h. Upon completion, the mixture was filtered, and the filtrate was diluted with water (200 mL) and extracted with EtOAc (200 mL×2). The combined organic layers were dried over Na₂SO₄, filtered, and concentrated in vacuo to afford 4-amino-3-cyclobutyl-1,3-dihydroquinoxalin-2-one (4.2 g) as a white solid, which was used in the next step directly without further purification. LC/MS (ESI⁺): m/z 218.1 [(M+H)⁺].

### Step 5:

To a stirred mixture of 4-amino-3-cyclobutyl-1,3-dihydroquinoxalin-2-one (4.2 g, 19.33 mmol) in EtOH (80 mL) was added ethyl 2-oxopropanoate (2.36 g, 20.30 mmol) and the resulting mixture was stirred at 25° C. for 1 h. Then 4 M HCl in EtOH (12 mL) was added and the mixture was stirred at 85° C. for 2 h. The mixture was concentrated in vacuo and the residue was diluted with water (200 mL), basified with saturated NaHCO₃ to pH-9, and extracted with EtOAc (150 mL×2). The combined organic layers were dried over Na₂SO₄, filtered and concentrated in vacuo. The residue was purified by reverse phase chromatography to afford ethyl 11-cyclobutyl-10-oxo-1,9-diazatricyclo[6.3.1.0^{4,12}]dodeca-2,4,6,8(12)-tetraene-2-carboxylate (1.75 g, 5.57 mmol, 28.8% yield) as a yellow solid. LC/MS (ESI⁺): m/z 299.1 [(M+H)⁺].

### Step 6:

To a stirred mixture of ethyl 11-cyclobutyl-10-oxo-1,9-diazatricyclo[6.3.1.0^{4,12}]dodeca-2,4,6,8(12)-tetraene-2-carboxylate (1.55 g, 5.20 mmol) in THF/MeOH/H₂O mixed solvents (28 mL, 3:3:1) was added LiOH·H₂O (654.1 mg, 15.59 mmol). The mixture was stirred at 45° C. for 13 h. Upon completion indicated by LC/MS, the mixture was concentrated in vacuo. The residue was diluted with water (200 mL), acidized with 1 M aqueous HCl to pH ˜4, and extracted with EtOAc (200 mL×2). The combined organic layers were dried over Na₂SO₄, filtered and concentrated in vacuo to afford 11-cyclobutyl-10-oxo-1,9-diazatricyclo[6.3.1.0^{4,12}]dodeca-2,4,6,8(12)-tetraene-2-carboxylic acid (1.4 g) as a yellow solid, which was used in the next step directly without further purification. LC/MS (ESI⁺): m/z 271.1 [(M+H)⁺].

### Step 7:

To a stirred mixture of 11-cyclobutyl-10-oxo-1,9-diazatricyclo[6.3.1.0^{4,12}]dodeca-2,4,6,8(12)-tetraene-2-carboxylic acid (1.7 g, 6.29 mmol) in DMF (30 mL) were added HATU (3.11 g, 8.18 mmol), DIEA (2.44 g, 18.87 mmol) and methyl 3-amino-5-fluoro-4-(methylamino)benzoate (1.50 g, 7.55 mmol). The resulting mixture was stirred at 25° C. for 4 h. Upon completion indicated by LC/MS, the mixture was diluted with water (120 mL) and extracted with EtOAc (100 mL×2). The combined organic layers were washed with brine, dried over Na₂SO₄, filtered and concentrated in vacuo to afford methyl 3-(3-cyclobutyl-2-oxo-2,3-dihydro-1H-pyrrolo[1,2,3-de]quinoxaline-5-carboxamido)-5-fluoro-4-(methylamino)benzoate (2.83 g) as a brown solid, which was used in the next step directly without further purification. LC/MS (ESI⁺): m/z 451.2 [(M+H)⁺].

### Step 8:

A mixture of methyl 3-[(11-cyclobutyl-10-oxo-1,9-diazatricyclo[6.3.1.0^{4,12}]dodeca-2,4,6,8(12)-tetraene-2-carbonyl)amino]-5-fluoro-4-(methylamino)benzoate (2.83 g, 6.28 mmol) in AcOH (40 mL) was stirred at 110° C. for 1 h. Upon completion, the mixture was cooled to 25° C. and concentrated in vacuo. The residue was diluted with water (120 mL), basified with saturated NaHCO₃, and extracted with EtOAc (100 mL×2). The combined organic layers were dried over Na₂SO₄, filtered, and concentrated in vacuo. The residue was purified by flash column chromatography on silica gel (eluting with 0-100% EA in PE) to afford methyl 2-(3-cyclobutyl-2-oxo-2,3-dihydro-1H-pyrrolo[1,2,3-de]quinoxalin-5-yl)-7-fluoro-1-methyl-1H-benzo[d]imidazole-5-carboxylate (1.4 g, 1.62 mmol, 25.8% yield) as a pink solid. LC/MS (ESI⁺): m/z 433.1 [(M+H)⁺].

### Step 9:

To a stirred solution of methyl 2-(11-cyclobutyl-10-oxo-1,9-diazatricyclo[6.3.1.0^{4,12}]dodeca-2,4,6,8(12)-tetraen-2-yl)-7-fluoro-1-methyl-benzimidazole-5-carboxylate (700 mg, 1.62 mmol) in THF (16 mL) was dropwise added 1.0 M borane in THF (4.86 mL, 4.86 mmol) at 25° C. The mixture was stirred at 25° C. for 1.5 h. Upon completion, the reaction was carefully quenched with MeOH and the mixture was concentrated in vacuo. The residue was purified by reverse phase chromatography to afford methyl 2-(11-cyclobutyl-1,9-diazatricyclo[6.3.1.0^{4,12}]dodeca-2,4,6,8(12)-tetraen-2-yl)-7-fluoro-1-methyl-benzimidazole-5-carboxylate (330 mg, 709.74 μmol, 43.8% yield) as a yellow solid. LC/MS (ESI⁺): m/z 419.2 [(M+H)⁺].

### Step 10:

A mixture of methyl 2-(11-cyclobutyl-1,9-diazatricyclo[6.3.1.0^{4,12}]dodeca-2,4,6,8(12)-tetraen-2-yl)-7-fluoro-1-methyl-benzimidazole-5-carboxylate (330 mg, 788.60 μmol) and LiOH·H₂O (99.3 mg, 2.37 mmol) in THF/MeOH/H₂O mixed solvents (9 mL, 4:4:1) was stirred at 40° C. for 1.5 h. Upon completion, the mixture was concentrated in vacuo. The residue was diluted with water (40 mL), acidized with 1 M HCl to pH ˜4, and extracted with EtOAc (40 mL×2). The combined organic layers were dried over Na₂SO₄, filtered and concentrated in vacuo to afford 2-(11-cyclobutyl-1,9-diazatricyclo[6.3.1.0^{4,12}]dodeca-2,4,6,8(12)-tetraen-2-yl)-7-fluoro-1-methyl-benzimidazole-5-carboxylic acid (310 mg) as a brown solid, which was used in the next step directly without further purification. LC/MS (ESI⁺): m/z 405.1 [(M+H)⁺].

### Step 11:

To a stirred solution of 2-(11-cyclobutyl-1,9-diazatricyclo[6.3.1.0^{4,12}]dodeca-2,4,6,8(12)-tetraen-2-yl)-7-fluoro-1-methyl-benzimidazole-5-carboxylic acid (320 mg, 791.23 μmol) in DMF (7 mL) were added HATU (391.1 mg, 1.03 mmol, DIEA (306.8 mg, 2.37 mmol) and tert-butyl ((1R,4R,7R)-2-azabicyclo[2.2.1]heptan-7-yl)carbamate (201.6 mg, 949.47 μmol). The mixture was stirred at 25° C. for 0.5 h. The mixture was purified by reverse phase chromatography to afford tert-butyl ((1R,4R,7R)-2-(2-(3-cyclobutyl-2,3-dihydro-1H-pyrrolo[1,2,3-de]quinoxalin-5-yl)-7-fluoro-1-methyl-1H-benzo[d]imidazole-5-carbonyl)-2-azabicyclo[2.2.1]heptan-7-yl)carbamate (350 mg, 555.36 μmol, 70.2% yield) as a brown solid. LC/MS (ESI⁺): m/z 599.3 [(M+H)⁺].

### Step 12:

To a stirred mixture of tert-butyl N-[(1R,4R,7R)-2-[2-(11-cyclobutyl-1,9-diazatricyclo[6.3.1.0^{4,12}]dodeca-2,4,6,8(12)-tetraen-2-yl)-7-fluoro-1-methyl-benzimidazole-5-carbonyl]-2-azabicyclo[2.2.1]heptan-7-yl]carbamate (350 mg, 584.59 μmol) in MeOH (2 mL) was added 4 M HCl in dioxane (6 mL). The mixture was stirred at 25° C. for 1 h. Upon completion, the mixture was concentrated in vacuo, and the residue was purified by prep-HPLC to afford [(1R,4R,7R)-7-amino-2-azabicyclo[2.2.1]heptan-2-yl]-[2-(11-cyclobutyl-1,9-diazatricyclo[6.3.1.0^{4,12}]dodeca-2,4,6,8(12)-tetraen-2-yl)-7-fluoro-1-methyl-benzimidazol-5-yl]methanone (266.1 mg, 507.02 μmol, 86.7% yield) as a white solid. LC/MS (ESI⁺): m/z 499.3 [(M+H)⁺]. ¹H NMR (400 MHz, DMSO-d₆) δ 7.79-7.61 (m, 1H), 7.34-7.17 (m, 1H), 6.95 (s, 1H), 6.93-6.83 (m, 2H), 6.36 (dd, J=7.1, 1.1 Hz, 1H), 6.06 (s, 1H), 5.28-5.20 (m, 1H), 4.12 (d, J=3.2 Hz, 3H), 3.79 (d, J=15.1 Hz, 1H), 3.52 (dd, J=12.0, 3.2 Hz, 2H), 3.46-3.22 (m, 2H), 3.13-3.00 (m, 1H), 2.38 (h, J=8.4 Hz, 1H), 2.30-2.18 (m, 1H), 2.03-1.85 (m, 2H), 1.85-1.70 (m, 2H), 1.61 (qd, J=9.4, 5.4 Hz, 1H), 1.55-1.26 (m, 4H), 0.85 (td, J=9.5, 5.8 Hz, 1H).

### Example 165 Preparation of ((1R,4R,7R)-7-amino-2-azabicyclo[2.2.1]heptan-2-yl)(2-(1-(cyclobutylmethyl)-1,8-dihydropyrrolo[3,2-g]indol-2-yl)-7-fluoro-1-methyl-1H-benzo[d]imidazol-5-yl)methanone

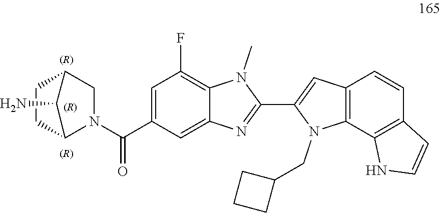

Prepared in analogous manner as for Example 148. LC/MS (ESI⁺): m/z 511.2 [(M+H)⁺]. ¹H NMR (400 MHz, DMSO-d₆) δ 11.36 (s, 1H), 7.76-7.59 (m, 1H), 7.37-7.18 (m, 4H), 7.11 (s, 1H), 6.59 (dd, J=3.0, 1.6 Hz, 1H), 4.98 (dd, J=7.3, 3.1 Hz, 2H), 4.10 (d, J=3.2 Hz, 3H), 3.76-3.62 (m, 1H), 3.52 (dt, J=11.0, 3.1 Hz, 1H), 3.20 (s, 1H), 3.06 (dd, J=13.2, 10.0 Hz, 1H), 2.24-2.09 (m, 1H), 2.04-1.89 (m, 2H), 1.78-1.44 (m, 6H), 1.44-1.31 (m, 3H).

### Example 166 Synthesis of (R)-(3-aminopiperidin-1-yl)(2-(2,3-dihydro-1H-pyrrolo[1,2,3-de]quinoxalin-5-yl)-1-methyl-1H-benzo[d]imidazol-5-yl)methanone

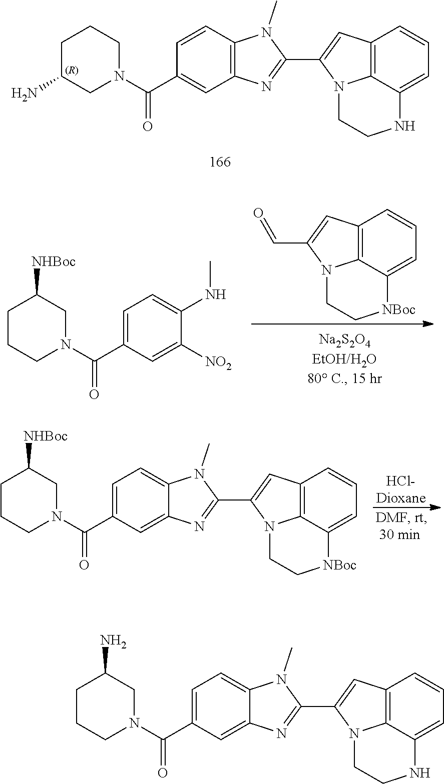

### Step 1:

A mixture of tert-butyl 2-formyl-1,9-diazatricyclo[6.3.1.04,12]dodeca-2,4(12), 5,7-tetraene-9-carboxylate (300.0 mg, 1.05 mmol, synthesized according to WO2014015905), tert-butyl N-[(3R)-1-[4-(methylamino)-3-nitro-benzoyl]-3-piperidyl]carbamate (436.1 mg, 1.15 mmol, intermediate of Example 1) and Na₂S₂O₄ (912.1 mg, 5.24 mmol) in ethanol/H₂O mixed solvents (10 mL, 1:1) was stirred at 80° C. for 15 h. Upon completion, the solvent was removed in vacuo and the residue was purified by prep-HPLC to afford tert-butyl N-[(3R)-1-[2-(1,9-diazatricyclo[6.3.1.04,12]dodeca-2,4(12), 5,7-tetraen-2-yl)-1-methyl-benzimidazole-5-carbonyl]-3-piperidyl]carbamate (300.0 mg, 582.96 μmol, 55.6% yield) as a yellow solid. LC/MS (ESI⁺): m/z 614.8 [(M+H)⁺].

### Step 2:

To a stirred mixture of tert-butyl 2-[5-[(3R)-3-(tert-butoxycarbonylamino)piperidine-1-carbonyl]-1-methyl-benzimidazol-2-yl]-1,9-diazatricyclo[6.3.1.04,12]dodeca-2,4(12), 5,7-tetraene-9-carboxylate (300.0 mg, 488.02 μmol) in DMF (1 mL) was added 4 M HCl in dioxane (3 mL). The mixture was stirred at RT for 30 min. Upon completion, the solvent was removed in vacuo and the residue was purified by prep-HPLC to afford [(3R)-3-amino-1-piperidyl]-[2-(1,9-diazatricyclo[6.3.1.04,12]dodeca-2,4(12), 5,7-tetraen-2-yl)-1-methyl-benzimidazol-5-yl]methanone (120 mg, 289.50 μmol, 59.3% yield) as a white solid. LC/MS (ESI⁺): m/z 414.8 [(M+H)⁺]. 1H NMR (400 MHz, DMSO-d₆) δ 7.75-7.70 (m, 2H), 7.35 (dd, J=8.2, 1.5 Hz, 1H), 7.04 (s, 1H), 6.93 (dd, J=8.1, 0.9 Hz, 1H), 6.90-6.81 (m, 1H), 6.36 (dd, J=7.1, 0.9 Hz, 1H), 6.12 (s, 1H), 4.62 (t, J=5.0 Hz, 2H), 4.05 (s, 3H), 3.56 (t, J=5.1 Hz, 2H), 2.90 (s, 3H), 1.94 (d, J=11.9 Hz, 1H), 1.70 (s, 1H), 1.47 (t, J=12.1 Hz, 2H).

### Example 167 Synthesis of ((R)-3-aminopiperidin-1-yl)(1-methyl-2-(3-methyl-2,3-dihydro-1H-pyrrolo[1,2,3-de]quinoxalin-5-yl)-1H-benzo[d]imidazol-5-yl)methanone

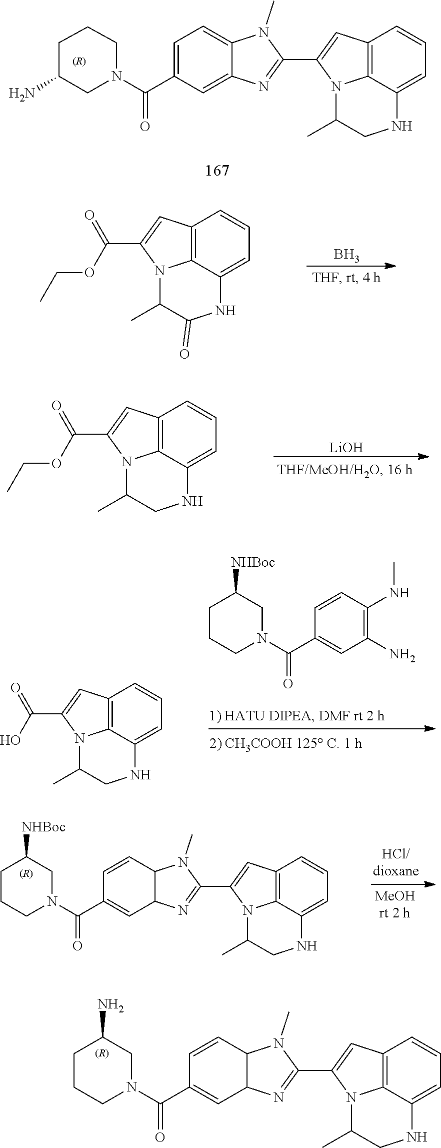

### Step 1:

To a stirred solution of ethyl 11-methyl-10-oxo-1,9-diazatricyclo[6.3.1.0^{4,12}]dodeca-2,4(12), 5,7-tetraene-2-carboxylate (1.4 g, 5.42 mmol, intermediate of Example 64) in anhydrous THF (20 mL) was dropwise added borane tetrahydrofuran (21.68 mmol, 21.7 mL) at 0° C. The reaction mixture was stirred at RT for 4 h, then quenched with MeOH at 0° C. and concentrated in vacuo. The residue was treated with 2 M HCl aqueous solution (6 mL), and the mixture was stirred at RT for 1 h before being basified with 4 M NaOH aqueous solution to pH ˜8. The mixture was extracted with DCM (30 mL×3), and the combined organic layers were dried over anhydrous sodium sulfate, then filtered. The filtrate was concentrated in vacuo and the residue was purified by silica gel flash column chromatography (eluting with 0-5% MeOH in DCM) to give ethyl 11-methyl-1,9-diazatricyclo[6.3.1.0^{4,12}]dodeca-2,4(12), 5,7-tetraene-2-carboxylate (1.2 g, 4.91 mmol, 90.6% yield) as a yellow solid. LC/MS (ESI⁺): m/z 245.1 [(M+H)⁺].

### Step 2:

To a stirred solution of ethyl 11-methyl-1,9-diazatricyclo[6.3.1.0^{4,12}]dodeca-2,4(12), 5,7-tetraene-2-carboxylate (1.2 g, 4.91 mmol) in THF/MeOH mixed solvents (30 mL, 2:1) was added a solution of LiOH (1.0 M, 20 mL). The mixture was stirred at RT for 16 h. Upon completion, the mixture was acidified to pH=5-6 with 3 M HCl aqueous solution, and then extracted with EA (100 mL×3). The organic phase was dried over anhydrous sodium sulfate and filtered. The filtrate was concentrated in vacuo to give 11-methyl-1,9-diazatricyclo[6.3.1.0^{4,12}]dodeca-2,4(12), 5,7-tetraene-2-carboxylic acid (1.0 g, 4.62 mmol, 94.1% yield) as a white solid. LC/MS (ESI⁺): m/z 217.1 [(M+H)⁺].

### Step 3:

To a stirred solution of 11-methyl-1,9-diazatricyclo[6.3.1.0^{4,12}]dodeca-2,4(12), 5,7-tetraene-2-carboxylic acid (200 mg, 924.92 μmol) in DMF (3 mL) were added DIPEA (358.6 mg, 2.77 mmol), HATU (527.5 mg, 1.39 mmol) and tert-butyl (R)-(1-(3-amino-4-(methylamino)benzoyl)piperidin-3-yl)carbamate (354.5 mg, 1.02 mmol, synthesized according to WO2014015905) and the resulting mixture was stirred at RT for 2 h. Upon completion, the mixture was diluted with EA, washed with brine, and dried over anhydrous sodium sulfate. After filtration and evaporation of the solvent in vacuo, the residue was redissolved in CH₃COOH (5 mL), and the mixture was stirred at 125° C. for 1 h. After cooling to RT, the mixture was concentrated in vacuo. The residue was diluted with EA (80 mL), washed with saturated Na₂CO₃ solution and dried over Na₂SO₄. After filtration and evaporation of the solvent in vacuo, the residue was purified by flash column chromatography on silica gel (eluting with 0-7% MeOH in DCM) to give tert-butyl ((3R)-1-(1-methyl-2-(3-methyl-2,3-dihydro-1H-pyrrolo[1,2,3-de]quinoxalin-5-yl)-1H-benzo[d]imidazole-5-carbonyl)piperidin-3-yl)carbamate (85 mg, 160.79 μmol, 17.4% yield) as a yellow solid. LC/MS (ESI⁺): m/z 529.3 [(M+H)⁺].

### Step 4:

To a stirred solution of tert-butyl N-[(3R)-1-[1-methyl-2-(11-methyl-1,9-diazatricyclo[6.3.1.0^{4,12}]dodeca-2,4,6,8(12)-tetraen-2-yl)benzimidazole-5-carbonyl]-3-piperidyl]carbamate (85 mg, 160.79 μmol) in MeOH (2 mL) was added 4 M HCl in dioxane (2 mL). The mixture was stirred at RT for 2 h. Upon completion, the mixture was basified to pH=8 with saturated Na₂CO₃ solution, and then the mixture was extracted with DCM (30 mL×3). The combined organic layers were dried over anhydrous sodium sulfate, filtered, and concentrated in vacuo. The residue was purified by flash column chromatography on silica gel (eluting with 0-10% MeOH in DCM) to give [(3R)-3-amino-1-piperidyl]-[1-methyl-2-(11-methyl-1,9-diazatricyclo[6.3.1.0^{4,12}]dodeca-2,4,6,8(12)-tetraen-2-yl)benzimidazol-5-yl]methanone (10 mg, 23.34 μmol, 14.5% yield) as a yellow solid. LC/MS (ESI⁺): m/z 429.3 [(M+H)⁺]. ¹H NMR (400 MHz, DMSO-d₆) δ 7.78-7.70 (m, 2H), 7.37 (dd, J=8.2, 1.5 Hz, 1H), 7.04 (s, 1H), 6.92 (d, J=7.9 Hz, 1H), 6.90-6.82 (m, 1H), 6.38 (d, J=7.2 Hz, 1H), 6.10 (d, J=3.0 Hz, 1H), 5.50 (d, J=7.2 Hz, 1H), 4.04 (s, 3H), 3.54 (dd, J=11.9, 3.2 Hz, 2H), 3.43-3.40 (m, 2H), 3.02 (s, 2H), 1.98 (d, J=7.2 Hz, 1H), 1.73 (s, 1H), 1.50 (s, 1H), 1.26 (dd, J=6.5, 2.0 Hz, 3H), 1.23 (s, 1H).

### Example 168 Preparation of ((1R,4R,7R)-7-amino-2-azabicyclo[2.2.1]heptan-2-yl)(2-(2,3-dihydro-1H-pyrrolo[1,2,3-de]quinoxalin-5-yl)-1-methyl-1H-benzo[d]imidazol-5-yl)methanone

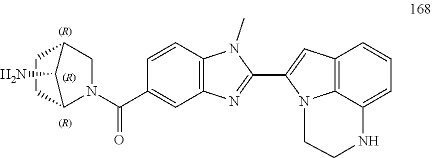

Prepared in analogous manner as for Example 167. LC/MS (ESI⁺): m/z 427.2 [(M+H)⁺]. ¹H NMR (400 MHz, DMSO-d₆) δ 7.87-7.65 (m, 2H), 7.48-7.37 (m, 1H), 7.03 (d, J=3.6 Hz, 1H), 6.93 (d, J=8.0 Hz, 1H), 6.85 (t, J=7.6 Hz, 1H), 6.40-6.33 (m, 1H), 6.11 (s, 1H), 4.63 (dd, J=6.4, 4.2 Hz, 2H), 4.04 (d, J=4.5 Hz, 3H), 3.73 (s, 1H), 3.65-3.48 (m, 3H), 3.19 (s, 1H), 3.12-2.95 (m, 1H), 2.25-2.08 (m, 1H), 2.03-1.81 (m, 2H), 1.76-1.62 (m, 1H), 1.51-1.29 (m, 1H).

### Example 169 Preparation of ((1R,4R,7R)-7-amino-2-azabicyclo[2.2.1]heptan-2-yl)(1-methyl-2-(3-methyl-2,3-dihydro-1H-pyrrolo[1,2,3-de]quinoxalin-5-yl)-1H-benzo[d]imidazol-5-yl)methanone

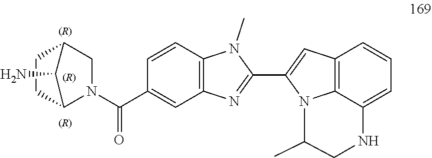

Prepared in analogous manner as for Example 167. LC/MS (ESI⁺): m/z 441.2 [(M+H)⁺]. ¹H NMR (400 MHz, DMSO-d₆) δ 7.86-7.64 (m, 2H), 7.49-7.37 (m, 1H), 7.03 (s, 1H), 6.95-6.82 (m, 2H), 6.38 (dd, J=7.2, 0.9 Hz, 1H), 6.08 (s, 1H), 5.51 (t, J=8.4 Hz, 1H), 4.03 (d, J=3.7 Hz, 3H), 3.79-3.48 (m, 3H), 3.44-3.17 (m, 2H), 3.11-2.96 (m, 1H), 2.25-2.11 (m, 1H), 2.03-1.82 (m, 2H), 1.71 (q, J=10.4 Hz, 1H), 1.49-1.32 (m, 1H), 1.25 (dd, J=7.9, 6.3 Hz, 3H).

### Example 170 Preparation of ((1R,4R,7R)-7-amino-2-azabicyclo[2.2.1]heptan-2-yl)(2-(3-cyclopropyl-2,3-dihydro-1H-pyrrolo[1,2,3-de]quinoxalin-5-yl)-5-methoxy-3-methylimidazo[1,2-a]pyridin-7-yl)methanone

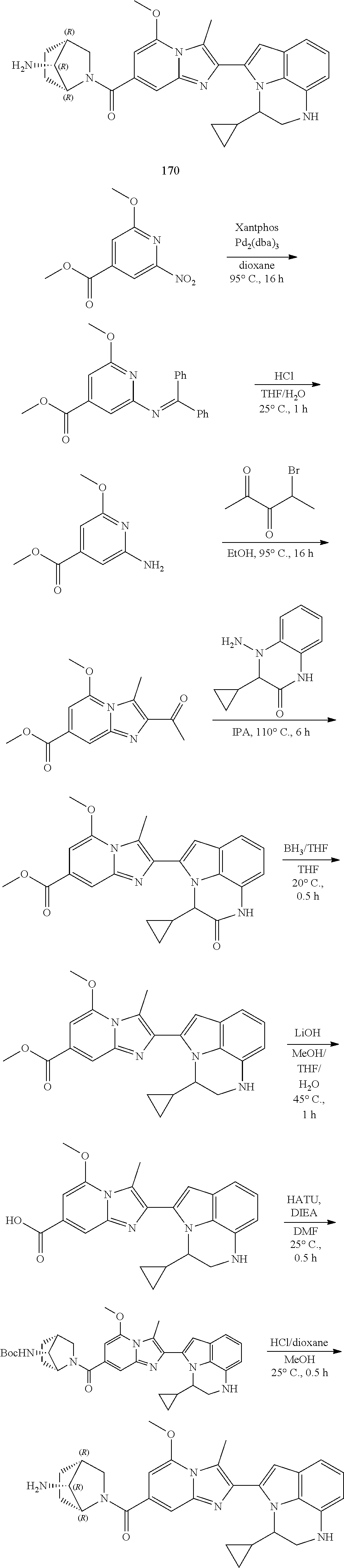

### Step 1:

To a stirred mixture of methyl 2-chloro-6-methoxyisonicotinate (5 g, 24.80 mmol), benzophenone imine (5.39 g, 29.76 mmol, 4.99 ml-) and Cs₂CO₃ (16.16 g, 49.60 mmol) in dioxane (125 ml-) were added (5-diphenylphosphanyl-9,9-dimethyl-xanthen-4-yl)-diphenyl-phosphane (861 mg, 1.49 mmol) and Pd₂(dba)₃ (681.3 mg, 744.02 μmol) under N₂ atmosphere. The mixture was stirred at 95° C. for 16 h. Upon completion, the mixture was diluted with water (300 mL) and extracted with EtOAc (250 mL×2). The combined organic layers were dried over Na₂SO₄, filtered, and concentrated in vacuo to afford methyl 2-((diphenylmethylene)amino)-6-methoxyisonicotinate (6.02 g, 17.36 mmol, 70.0% yield) as a brown oil, which was used in the next step directly without further purification. LC/MS (ESI⁺): m/z 347.1 [(M+H)⁺].

### Step 2:

To a stirred mixture of methyl 2-(benzhydrylideneamino)-6-methoxy-pyridine-4-carboxylate (8.59 g, 24.80 mmol) in THF/water mixed solvents (120 mL, 2:1) was added concentrated HCl (11 mL). The resulting mixture was stirred at 25° C. for 1 h. Upon completion, the mixture was basified with saturated NaHCO₃ to pH ˜9, then diluted with water (100 mL) and extracted with EtOAc (150 mL×2). The combined organic layers were dried over Na₂SO₄, filtered, and concentrated in vacuo. The residue was purified by reverse phase chromatography to afford methyl 2-amino-6-methoxy-pyridine-4-carboxylate (2.44 g, 13.34 mmol, 53.7% yield) as a brown solid. LC/MS (ESI⁺): m/z 183.1 [(M+H)⁺].

### Step 3:

A mixture of methyl 2-amino-6-methoxy-pyridine-4-carboxylate (2.7 g, 14.82 mmol) and 4-bromopentane-2,3-dione (5.31 g, 29.64 mmol) in EtOH (60 mL) was stirred at 95° C. for 16 h. Upon completion, the mixture was concentrated in vacuo. The residue was diluted with EtOAc (200 mL), then basified with saturated NaHCO₃, and washed with water (150 mL). The organic layer was dried over Na₂SO₄, filtered, and concentrated in vacuo. The residue was purified by reverse phase chromatography to afford methyl 2-acetyl-5-methoxy-3-methylimidazo[1,2-a]pyridine-7-carboxylate (0.602 g, 2.29 mmol, 15.4% yield) as a brown solid. LC/MS (ESI⁺): m/z 263.1 [(M+H)⁺].

### Step 4:

A mixture of 4-amino-3-cyclopropyl-1,3-dihydroquinoxalin-2-one (900 mg, 4.43 mmol) and methyl 2-acetyl-5-methoxy-3-methylimidazo[1,2-a]pyridine-7-carboxylate (1 g, 3.81 mmol) in IPA (20 mL) was stirred at 110° C. for 6 h. Upon completion, the mixture was concentrated in vacuo, and the residue was purified by reverse phase chromatography to afford methyl 2-(3-cyclopropyl-2-oxo-2,3-dihydro-1H-pyrrolo[1,2,3-de]quinoxalin-5-yl)-5-methoxy-3-methylimidazo[1,2-a]pyridine-7-carboxylate (250 mg, 580.78 μmol, 13.1% yield) as a brown solid. LC/MS (ESI⁺): m/z 431.2 [(M+H)⁺].

### Step 5:

To a stirred solution of methyl 2-(11-cyclopropyl-10-oxo-1,9-diazatricyclo[6.3.1.0^{4,12}]dodeca-2,4,6,8(12)-tetraen-2-yl)-5-methoxy-3-methyl-imidazo[1,2-a]pyridine-7-carboxylate (500 mg, 1.16 mmol) in THF (10 mL) was added 1.0 M BH₃ in THF (3.48 mL, 3.48 mmol) at 20° C. The mixture was stirred at 20° C. for 0.5 h. Upon completion, the mixture was carefully quenched with MeOH. Then the mixture was concentrated in vacuo, and the residue was purified by flash column chromatography on silica gel (eluting with 0-40% EA in PE) to afford methyl 2-(11-cyclopropyl-1,9-diazatricyclo[6.3.1.0^{4,12}]dodeca-2,4,6,8(12)-tetraen-2-yl)-5-methoxy-3-methyl-imidazo[1,2-a]pyridine-7-carboxylate (38 mg, 90.76 μmol, 7.8% yield) as a yellow solid. LC/MS (ESI⁺): m/z 417.2 [(M+H)⁺].

### Step 6:

To a stirred mixture of methyl 2-(11-cyclopropyl-1,9-diazatricyclo[6.3.1.0^{4,12}]dodeca-2,4,6,8(12)-tetraen-2-yl)-5-methoxy-3-methyl-imidazo[1,2-a]pyridine-7-carboxylate (42 mg, 100.85 μmol) in THF/MeOH/H₂O mixed solvents (3.5 mL, 3:3:1) was added LiOH·H₂O (12.7 mg, 302.54 μmol). The mixture was stirred at 45° C. for 1 h. Upon completion, the mixture was concentrated in vacuo. The residue was diluted with water (20 mL), acidized with 1 M HCl to pH ˜5, and extracted with EtOAc (20 mL×2). The combined organic layers were dried over Na₂SO₄, filtered, and concentrated in vacuo to afford 2-(11-cyclopropyl-1,9-diazatricyclo[6.3.1.0^{4,12}]dodeca-2,4,6,8(12)-tetraen-2-yl)-5-methoxy-3-methyl-imidazo[1,2-a]pyridine-7-carboxylic acid (36 mg, 89.45 μmol, 88.7% yield) as a yellow solid, which was used in the next step directly without further purification. LC/MS (ESI⁺): m/z 403.1 [(M+H)⁺].

### Step 7:

To a stirred mixture of 2-(11-cyclopropyl-1,9-diazatricyclo[6.3.1.0^{4,12}]dodeca-2,4,6,8(12)-tetraen-2-yl)-5-methoxy-3-methyl-imidazo[1,2-a]pyridine-7-carboxylic acid (40 mg, 99.39 μmol) in DMF (3 mL) were added HATU (49.1 mg, 129.21 μmol), DIEA (38.5 mg, 298.18 μmol), and tert-butyl ((1R,4R,7R)-2-azabicyclo[2.2.1]heptan-7-yl)carbamate (25.3 mg, 119.27 μmol). The mixture was stirred at 25° C. for 0.5 h. Upon completion, the mixture was diluted with water (30 mL) and extracted with EtOAc (30 mL×2). The combined organic layers were dried over Na₂SO₄, filtered, and concentrated in vacuo. The residue was purified by reverse phase chromatography to afford tert-butyl ((1R,4R,7R)-2-(2-(3-cyclopropyl-2,3-dihydro-1H-pyrrolo[1,2,3-de]quinoxalin-5-yl)-5-methoxy-3-methylimidazo[1,2-a]pyridine-7-carbonyl)-2-azabicyclo[2.2.1]heptan-7-yl)carbamate (45 mg, 75.41 μmol, 75.9% yield) as a brown solid. LC/MS (ESI⁺): m/z 597.3 [(M+H)⁺].

### Step 8:

To a stirred solution of tert-butyl N-[(1R,4R,7R)-2-[2-(11-cyclopropyl-1,9-diazatricyclo[6.3.1.0^{4,12}]dodeca-2,4,6,8(12)-tetraen-2-yl)-5-methoxy-3-methyl-imidazo[1,2-a]pyridine-7-carbonyl]-2-azabicyclo[2.2.1]heptan-7 yl]carbamate (50 mg, 83.79 mol) in MeOH (1.2 mL) was added 4.0 M HCl in dioxane (2.5 mL). The mixture was stirred at 25° C. for 0.5 h. Upon completion, the mixture was concentrated in vacuo, and the residue was purified by prep-HPLC to afford [(1R,4R,7R)-7-amino-2-azabicyclo[2.2.1]heptan-2-yl]-[2-(11-cyclopropyl-1,9-diazatricyclo[6.3.1.0^{4,12}]dodeca-2,4,6,8(12)-tetraen-2-yl)-5-methoxy-3-methyl-imidazo[1,2-a]pyridin-7-yl]methanone (20 mg, 40.36 μmol, 48.2% yield) as a yellow solid. LC/MS (ESI⁺): m/z 497.2 [(M+H)⁺]. ¹H NMR (400 MHz, DMSO-d₆) δ 7.97 (d, J=4.6 Hz, 1H), 7.44-7.23 (m, 1H), 6.82-6.74 (m, 2H), 6.47-6.41 (m, 1H), 6.28 (dd, J=6.3, 1.9 Hz, 1H), 5.97 (s, 1H), 4.82 (dd, J=9.3, 3.7 Hz, 1H), 4.18 (d, J=1.8 Hz, 3H), 3.80 (d, J=13.9 Hz, 1H), 3.62-3.48 (m, 3H), 3.23-3.04 (in, 2H), 2.39 (s, 3H), 2.27-2.14 (m, 1H), 2.05-1.63 (m, 3H), 1.48-1.34 (m, 1H), 1.01-0.89 (m, 1H), 0.33-0.24 (m, 1H), 0.21-0.12 (m, 1H), 0.02-−0.08 (in, 1H), −0.32-−0.43 (in, 1H).

### Biological Assays

### Biological Example 1. PAD4 Ammonia Release Assay

### Reagents and Consumables:

| Reagents/Supplies | Vendor | Cat# | Lot# |
|---|---|---|---|
| Protein arginine deiminase | Regor | Produced by Viva, | |
| 4 (PAD4) | | Lot 20200604 | |
| Protein arginine deiminase | Regor | Produced by Viva, | |
| 2 (PAD2) | | Lot 20190724 | |
| N^{α}-Benzoyl-L-arginine | Sigma | B4500 | / |
| Ethyl Ester (BAEE) | | | |
| Phthaldialdehyde (OPA) | Sigma | P0657-1G | / |
| 1,4-Dithiothreitol (DTT) | Sigma | 43816 | / |
| piperazineethanesulfonic | Gibico | 15630080 | / |
| acid (HEPES) | | | |
| Bovine serum albumin | Sigma | A1933-5G | / |
| (BSA) | | | |
| Sodium chloride (NaCl) | Invitrogen | AM9759 | 755693 |
| Calcium chloride (CaCl₂) | Sigma | 21115-100ML | BCBZ8220 |
| tris(2-chloroethyl) | Sigma | 75259-1g | / |
| phosphate (TCEP) | | | |
| Dimethyl sulfoxide | Sigma | D8418 | SHBK2703 |
| (DMSO) | | | |
| Triton X-100 | Sigma | X100-100ML | SLBX9437 |
| Black 384 well microplate | Corning | 3573 | / |

### Procedures

1) Compound preparation: The compounds were diluted to 80× final concentrations.
2) The compounds were transferred to a 384-well assay plate by Tecan. DMSO: 1.25%.
3) Preparation of 1×Assay buffer: 100 mM HEPES, PH=8.0, 2 mM CaCl₂, 50 mM NaCl, 0.1 mM TCEP, 0.6 mg/ml BSA.
4) 2× enzyme working solution (PAD4:20 nM) was prepared with assay buffer, and 10 μL of the obtained working solution were added to the 384-well assay plates.
5) The plate was centrifuged at 1000 rpm for 1 min.
6) After the centrifuge, the plate was incubated at RT for 15 min.
7) 2×BAEE substrate mix (600 μM) was prepared with assay buffer and 10 μL of the obtained substrate mix were added to each well.
8) The plate was centrifuged at 1000 rpm for 1 min and then incubated at RT for 60 min.
9) 1.5× detection mix (DTT:4 mM, OPA:3 mM, EDTA: 30 mM) was prepared, and 10 μL of the mix were added to the plate.
10) The plate was centrifuged at 1000 rpm for 1 min and then incubated at RT for 120 min.
11) The 384-well plates were placed into the Envision (PerkinElmer) and the assay data were collected.

### Data Analysis

The percent (%) inhibition at each concentration of compound was calculated based on and relative to the signal in the HPE (50 μM ref. compound) and ZPE (1.25% DMSO) wells included within each assay plate. The HPE worked as 100% inhibition, and the ZPE worked as 0% inhibition. The concentrations and % inhibition values for tested compounds were plotted and the concentration of compound required for 50% inhibition (IC₅₀) was determined with a Four-parameter logistic dose response equation. The endpoint value (IC₅₀) for the reference compound was evaluated in each experiment as a quality control measure. If the endpoint value is within 3-fold of the expected value, then the experiment quality is deemed as acceptable. The results are provided in Table 1 below. ≤0.1 μM=“++++”; >0.1 and ≤0.5 μM=“+++”; >0.5 and ≤5.0 μM=“++”; >5.0 μM=“+”. “−”=not available.

TABLE 1

| Example | Average IC₅₀ | Example | Average IC₅₀ |
|---|---|---|---|
| No. | Abs (nM) | No. | Abs (nM) |
| 1 | ++ | 55 | ++ |
| 2 | + | 56 | + |
| 3 | ++ | 57 | + |
| 4 | ++ | 58 | ++ |
| 5 | ++ | 59 | ++++ |
| 6 | ++ | 60 | + |
| 7 | ++ | 61 | + |
| 8 | + | 62 | + |
| 9 | + | 63 | + |
| 10 | ++ | 64 | +++ |
| 11 | ++ | 65 | ++ |
| 12 | +++ | 66 | ++ |
| 13 | +++ | 67 | + |
| 14 | ++ | 68 | +++ |
| 15 | + | 69 | ++++ |
| 16 | + | 70 | + |
| 17 | + | 71 | ++++ |
| 18 | ++ | 72 | +++ |
| 19 | + | 73 | + |
| 20 | ++ | 74 | ++++ |
| 21 | ++ | 75 | +++ |
| 22 | +++ | 76 | − |
| 23 | +++ | 77 | +++ |
| 24 | + | 78 | ++ |
| 25 | + | 79 | +++ |
| 26 | ++ | 80 | +++ |
| 27 | ++ | 81 | ++ |
| 28 | ++ | 82 | ++++ |
| 29 | ++ | 83 | +++ |
| 30 | + | 84 | +++ |
| 31 | + | 85 | +++ |
| 32 | ++ | 86 | + |
| 33 | ++ | 87 | +++ |
| 34 | ++++ | 88 | +++ |
| 35 | +++ | 89 | +++ |
| 36 | +++ | 90 | +++ |
| 37 | ++++ | 91 | +++ |
| 38 | ++++ | 92 | +++ |
| 39 | +++ | 93 | +++ |
| 40 | +++ | 94 | ++++ |
| 41 | +++ | 95 | +++ |
| 42 | +++ | 96 | +++ |
| 43 | ++++ | 97 | + |
| 44 | +++ | 98 | ++++ |
| 45 | + | 99 | +++ |
| 46 | ++++ | 100 | +++ |
| 47 | +++ | 101 | + |
| 48 | +++ | 102 | +++ |
| 49 | +++ | 103 | +++ |
| 50 | ++++ | 104 | +++ |
| 51 | +++ | 105 | ++ |
| 52 | + | 106 | +++ |
| 53 | + | 107 | +++ |
| 54 | + | 108 | ++++ |
| 109 | ++ | 141 | ++ |
| 110 | ++++ | 142 | ++ |
| 111 | ++++ | 143 | +++ |
| 112 | ++++ | 144 | +++ |
| 113 | ++ | 145 | ++ |
| 114 | ++++ | 146 | + |
| 115 | ++++ | 147 | ++++ |
| 116 | ++ | 148 | ++++ |
| 117 | + | 149 | ++ |
| 118 | +++ | 150 | +++ |
| 119 | + | 151 | ++ |
| 120 | + | 152 | +++ |
| 121 | ++++ | 153 | +++ |
| 122 | +++ | 154 | ++ |
| 123 | ++++ | 155 | +++ |
| 124 | ++ | 156 | ++++ |
| 125 | + | 157 | ++++ |
| 126 | ++ | 158 | ++++ |
| 127 | ++ | 159 | ++ |
| 128 | +++ | 160 | ++ |
| 129 | +++ | 161 | ++++ |
| 130 | +++ | 162 | ++ |
| 131 | +++ | 163 | +++ |
| 132 | +++ | 164 | +++ |
| 133 | + | 165 | ++++ |
| 134 | + | 166 | + |
| 135 | + | 167 | ++ |
| 136 | + | 168 | ++ |
| 137 | ++ | 169 | ++ |
| 138 | ++ | 170 | + |
| 139 | ++ | 140 | +++ |

### Biological Example 2. PAD2 Ammonia Release Assay

### Procedures

1) Compound preparation: The compounds were diluted to 80× final concentrations.
2) The compounds were transferred to a 384-well assay plate by Tecan. DMSO: 1.25%.
3) Preparation of 1×Assay buffer: 100 mM HEPES, PH=8.0, 2 mM CaCl₂, 50 mM NaCl, 0.1 mM TCEP, 0.6 mg/mL BSA.
4) 2× enzyme working solution (PAD2:20 nM) was prepared with assay buffer, and 10 μL of the obtained working solution were added to the 384-well assay plates.
5) The plate was centrifuged at 1000 rpm for 1 min.
6) After the centrifuge, the plate was incubated at RT for 15 min.
7) 2×BAEE substrate mix (600 μM) was prepared with assay buffer and 10 μL of the obtained substrate mix were added to each well.
8) The plate was centrifuged at 1000 rpm for 1 min and then incubated at RT for 60 min.
9) 1.5× detection mix (DTT:4 mM, OPA:3 mM, EDTA: 30 mM) was prepared, and 10 μL of the mix were added to the plate.
10) The plate was centrifuged at 1000 rpm for 1 min and then incubated at RT for 120 min.
11) The 384-well plates were placed into the Envision (PerkinElmer) and the assay data were collected.

### Data Analysis

The percent (%) inhibition at each concentration of compound was calculated based on and relative to the signal in the HPE (no PAD2 enzyme) and ZPE (1.25% DMSO) wells included within each assay plate. The HPE worked as 100% inhibition, and the ZPE worked as 0% inhibition. The concentrations and % inhibition values for tested compounds were plotted and the concentration of compound required for 50% inhibition (IC₅₀) was determined with a Four-parameter logistic dose response equation. The endpoint value (IC₅₀) for the reference compound was evaluated in each experiment as a quality control measure. If the endpoint value is within 3-fold of the expected value, then the experiment quality is deemed as acceptable.

It was found that the compounds of the present invention did not actively inhibit PAD2.

Biological Example 3. dHL-60 Cit-H3 ELISA Assay

### Reagents and Consumables:

| Name | Vendor | Cat# |
|---|---|---|
| human leukemia cell (HL-60) | ATCC | CCL-240 |
| modification of Dulbecco's Modified Eagle's | ATCC | 30-2001 |
| Medium (IMDM) | | |
| Fetal bovine serum (FBS) | Gibco | 10099141 |
| Dimethylformamide (DMF) | Merck | D4551 |
| Calcimicin (A23187) (25 mM) | Cayman | 11016 |
| 96 well flat bottom TC treated plate | Corning | 3599 |
| Citrullinated Histone H3 (Clone 11D3) ELISA | Cayman | 501620 |
| kit | | |

### Procedures

Cell culture: HL-60 cells were grown in IMDM+20% FBS+1% PS. The suspension cells were split into 1:5 with fresh media every 2-3 days to maintain cell density between 1×10⁵ to 1×10⁶ viable cells/mL.
HL-60 cells differentiation: HL-60 cells were diluted to 1×10⁵ cells/mL and differentiated into neutrophil-like cells with 100 mM DMF treatment for 72 hours in flask at 37° C., 5% CO₂.
dHL-60 Cells plating:
1) HL-60 cells were collected, and the plate was centrifuged at 1000 rpm for 5 min.
2) The HL-60 cells were diluted to 1.25×10⁶ cells/mL and plated into a 96-well plate with 80 μL/well (i.e., 1×10⁵/well). The plate was then incubated in the incubator (5% CO₂, 37° C.) for 30 min to allow cells to adhere.
   Compound treatment:
1) Preparation of compound serial dilution (source plate 500×, final DMSO concentration: 0.2%): briefly, compounds were dissolved in 100% DMSO to a concentration of 20 mM (the stock solution) and a 3-fold serial dilution with 8-point doses was performed.
2) A 10× compound solution was pipetted up and down; and 10 μL/well of compounds were added into each well and incubated for 30 min.
   Stimulation with 25 μM A23187:
1) A solution of A23187 (250 μM) in complete IMDM (IMDM+10% FBS+1% PS) containing 150 U/mL S7 Nuclease was prepared. The obtained solution was added 10 μL/well into each well and incubated for 3 h in the incubator (5% CO₂, 37° C.).
2) The dHL-60 cells treated with 10 μL/well complete IMDM without A23187 worked as negative control.
   Sample preparation:
1) EDTA (0.5 M, 2 μL/well) were added to all the wells to stop the reaction.
2) The plate was centrifuged at 1000 rpm for 5 min to collect supernatant.
3) The supernatant was then diluted at least 1:2 before adding to the ELISA plate for citrullinated H3 analysis.

### Citrullinated Histone H3 ELISA Standard (Item No. 401444)

The standard was reconstituted with 2 mL of Assay buffer to 500 ng/mL. The reconstituted standard was relatively unstable at 4° C. and should be used within 3 hr after reconstitution.

### Performing the Assay

1) 100 μL of the standards or diluted samples were added to the wells on the ELISA plate.
2) The plate was covered with Cover Sheet and incubated for 2 h at room temperaturert on an orbital shaker.

### Anti-Histone H3 HRP Conjugate (Item No. 401620, 10×)

On the day of the assay, the reagent was thawed at room temperaturert. For one plate, 1.2 mL of HRP Conjugate were diluted into 10.8 mL of assay buffer. A diluted HRP Conjugate was prepared shortly before use.

### Addition of HRP Conjugate and Second Incubation

1) The wells were empties and rinsed four times with 1× wash buffer with 300 μL/well (Low speed). The plate was inverted between wash steps to empty the plate. After the last wash, the inverted plate was gently tapped on absorbent paper to remove the residual wash buffer.
2) 100 μL of the HRP Conjugate working solution were added to each well of the plate.
3) The plate was covered with cover sheet and incubated for 1 h at RT on an orbital shaker.

### Development of the Plate

1) The wells were empties and rinsed four times with 1× wash buffer with 300 μL/well (low speed). The plate was inverted between wash steps to empty the plate. After the last wash, the inverted plate was gently tapped on absorbent paper to remove the residual wash buffer.
2) 100 μL of TMB Substrate solution were added to each well of the plate.
3) The plate was covered with cover sheet and incubation for 30 min at RT on an orbital shaker.
4) Do not wash the plate. 100 uL of HRP STOP solution were added to each well of the plate.

### Reading the Plate:

1) Wipe the bottom of the plate with a clean tissue to remove fingerprints, dirt, etc.
2) Read the plate at a wavelength of 450 nm.

### Data Analysis:

The percent (%) inhibition at each concentration of compound was calculated based on and relative to the signal in the HPE and ZPE wells contained within each assay plate. The HPE wells worked as 0% inhibition, and the ZPE wells didn't contain any compound but rather DMSO (final concentration=0.1%) as 100% inhibition. The concentrations and % inhibition values for tested compounds were plotted and the concentration of compound required for 50% inhibition (IC₅₀) was determined with a Four-parameter logistic dose response equation. The endpoint value (IC₅₀) for the reference peptide/compound was evaluated in each experiment as a quality control measure. If the endpoint value is within 3-fold of the expected value then the experiment is deemed acceptable. The results of representative compounds of the present invention are provided in Table 2 below. ≤0.1 M=“++++”; >0.1 and ≤0.5 μM=“+++”; >0.5 and ≤5.0 μM=“++”; >5.0 μM=“+”. “−”=not available.

TABLE 2

| Example | Average IC₅₀ | Example | Average IC₅₀ |
|---|---|---|---|
| No. | Abs (nM) | No. | Abs (nM) |
| 2 | ++ | 69 | ++++ |
| 10 | ++ | 71 | ++++ |
| 12 | ++ | 72 | +++ |
| 13 | ++ | 74 | ++++ |
| 14 | ++ | 75 | +++ |
| 23 | +++ | 80 | +++ |
| 34 | ++ | 81 | + |
| 37 | +++ | 82 | ++++ |
| 38 | ++ | 85 | ++++ |
| 40 | ++ | 86 | ++ |
| 42 | ++ | 87 | ++++ |
| 43 | ++ | 88 | +++ |
| 44 | ++ | 91 | +++ |
| 46 | ++ | 92 | ++++ |
| 47 | ++ | 94 | +++ |
| 48 | +++ | 95 | +++ |
| 49 | ++ | 96 | ++++ |
| 50 | +++ | 100 | +++ |
| 59 | ++ | 103 | ++++ |
| 64 | +++ | 104 | ++++ |
| 68 | +++ | 106 | ++++ |
| 110 | ++++ | 107 | +++ |
| 111 | ++++ | 147 | +++ |
| 112 | ++++ | 148 | ++++ |
| 114 | ++++ | 153 | +++ |
| 115 | ++++ | 156 | ++++ |
| 122 | +++ | 157 | ++++ |
| 123 | ++++ | 158 | ++++ |
| 130 | +++ | 161 | ++++ |
| 140 | ++ | 164 | ++++ |

### Biological Example 4. 4T1 CECN ELISA Assay

### Reagents and Consumables:

| Matrix | Vendor | Cat# |
|---|---|---|
| 4T1 (TNBC cell line) | ATCC | CRL-2539 |
| DMEM + 1 mM CaCl₂ | Gibco | 11995-065 |
| FBS | Gibco | 10099141 |
| A23187 (50 mM) | Cayman | 11016 |
| HEPES (10 mM) | Gibco | 15630-080 |
| PS | Gibco | 15140-122 |
| 96 well flat bottom TC treated plate | Corning | 3599 |
| Citrullinated Histone H3 (Clone 11D3) | Cayman | 501620 |
| ELISA kit | | |

### Procedures

Cell culture: 4T1 cells were grown in DMEM+10% FBS+1% HEPES+1% PS.
Cells plating:
1) 4T1 cells were collected and the plate was centrifuged at 1000 rpm for 5 min.
2) The 4T1 cells were diluted to 0.5×10⁶ cells/mL and plated into a 96-well plate with 100 μL/well. The plate was then incubated in the incubator (5% CO₂, 37° C.) for overnight to allow cells to adhere, exchanged fresh culture medium with 100 μL/well.
   Compound treatment:
1) Preparation of compound serial dilution (source plate 500×, final DMSO concentration: 0.2%): briefly, compounds were dissolved in 100% DMSO to a concentration of 20 mM (the stock solution) and a 3-fold serial dilution with 8-point doses was performed.
2) 10× compound solution was pipetted up and down; and 10 μL/well of compounds were added into each well and incubated for 30 min.
   Stimulation with 50 μM A23187:
1) A solution of A23187 (500 μM) in complete DMDM (DMDM+10% FBS+1% PS+1 mM CaCl₂) containing 150 U/mL S7 Nuclease was prepared. The obtained solution was added 10 μL/well into each well and incubated for 4 h in the incubator (5% CO₂, 37° C.).
2) The 4T1 cells treated with 10 μL/well complete DMDM without A23187 worked as negative control.
   Sample preparation:
1) EDTA (0.5 M, 2 μL/well) were added to all the wells to stop the reaction.
2) The plate was centrifuged at 1000 rpm for 5 min to collect supernatant.
3) The supernatant was then diluted at least 1:2 before adding to the ELISA plate for citrullinated H3 analysis.

### Citrullinated Histone H3 ELISA Standard (Item No. 401444)

The standard was reconstituted with 2 mL of Assay buffer to 500 ng/mL. The reconstituted standard was relatively unstable at 4° C. and should be used within 3 hr after reconstitution.

### Performing the Assay

1) 100 μL of the standards or diluted samples were added to the wells on the ELISA plate.
2) The plate was covered with Cover Sheet and incubated for 2 h at RT on an orbital shaker.

### Anti-Histone H3 HRP Conjugate (Item No. 401620, 10×)

On the day of the assay, the reagent was thawed at rt. For one plate, 1.2 mL of HRP Conjugate were diluted into 10.8 mL of assay buffer. A diluted HRP Conjugate was prepared shortly before use.

### Addition of HRP Conjugate and Second Incubation

1) The wells were empties and rinsed four times with 1× wash buffer with 300 μL/well (Low speed). The plate was inverted between wash steps to empty the plate. After the last wash, the inverted plate was gently tapped on absorbent paper to remove the residual wash buffer.
2) 100 μL of the HRP Conjugate working solution were added to each well of the plate.
3) The plate was covered with cover sheet and incubated for 1 h at RT on an orbital shaker.

### Development of the Plate

1) The wells were empties and rinsed four times with 1× wash buffer with 300 μL/well (low speed). The plate was inverted between wash steps to empty the plate. After the last wash, the inverted plate was gently tapped on absorbent paper to remove the residual wash buffer.
2) 100 μL of TMB Substrate solution were added to each well of the plate.
3) The plate was covered with cover sheet and incubation for 30 min at RT on an orbital shaker.
4) Do not wash the plate. 100 μL of HRP STOP solution were added to each well of the plate.

### Reading the Plate:

1) Wipe the bottom of the plate with a clean tissue to remove fingerprints, dirt, etc.
2) Read the plate at a wavelength of 450 nm.

### Data Analysis:

The percent (%) inhibition at each concentration of compound was calculated based on and relative to the signal in the HPE and ZPE wells contained within each assay plate. The HPE wells worked as 0% inhibition, and the ZPE wells didn't contain any compound but rather DMSO (final concentration=0.1%) as 100% inhibition. The concentrations and % inhibition values for tested compounds were plotted and the concentration of compound required for 50% inhibition (IC₅₀) was determined with a Four-parameter logistic dose response equation. The endpoint value (IC₅₀) for the reference peptide/compound was evaluated in each experiment as a quality control measure. If the endpoint value is within 3-fold of the expected value then the experiment is deemed acceptable. The results of representative compounds of the present invention are provided in Table 3 below. ≤0.1 μM=“++++”; >0.1 and ≤0.5 μM=“+++”; >0.5 and ≤5.0 μM=“++”; >5.0 μM=“+”. “−”=not available.

TABLE 3

| Example No. | Average IC₅₀ Abs (nM) |
|---|---|
| 75 | +++ |
| 82 | ++++ |
| 86 | + |
| 87 | ++++ |
| 95 | ++++ |
| 96 | +++ |

### Biological Example 5. PAD4 Activity Comparison

As shown in Table 4 below, the PAD4 inhibition activity (IC₅₀) of compound A is about 15.2 μM and that of compound B is about 7.3 μM. It is surprising to find that the IC₅₀ of both compounds were improved more than 50% when the oxygen atom in the tricyclic ring was replaced with a “NH” group. See Examples 166 and 167, the IC₅₀ of which are 9.53 μM and 4.09 μM. It is also found that the insertion of an R² group (e.g., —OCH₃) also improved the potency. See Examples 65 and 167; and Examples 2 and 166. It is further found that the election of different R¹ groups (in particular a chiral moiety) may also significantly improve potency. See Examples 166 and 168; and Examples 167 and 169. Thus, by elections of a combined groups at different positions, the IC₅₀ of the compounds of the present disclosure were significantly improved. See Examples 2, 65, 168, 169, etc. The IC₅₀ of Example 102 of the present disclosure is about 0.2 μM while the IC₅₀ of Example 104 is about 0.1 μM, which are about 35-100 folds more potent than compounds A and B.

TABLE 4

| | | PAD4 |
|---|---|---|
| Cmpd No. | Structure | Ammonia assay IC50 (μM) |
| A | 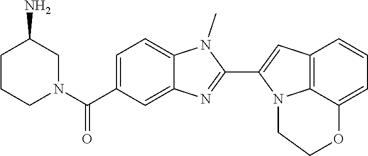 [SMILES: Cn1c(-c2cc3cccc4c3n2CCO4)nc2cc(C(=O)N3CCC[C@@H](N)C3)ccc21] | 15.2 |
| B | 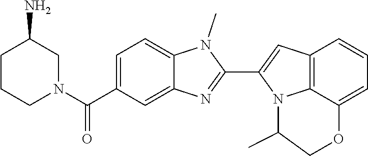 [SMILES: CC1COc2cccc3cc(-c4nc5cc(C(=O)N6CCC[C@@H](N)C6)ccc5n4C)n1c23] | 7.28 |
| Example 166 | 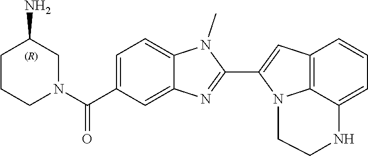 | 9.53 |
| Example 167 | 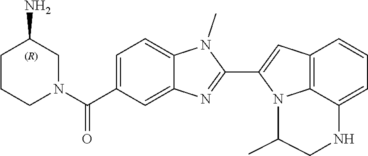 | 4.09 |
| Example 65 | 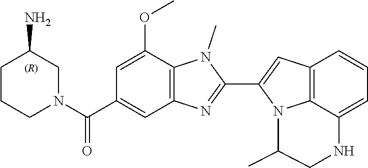 | 2.6 |
| Example 2 | 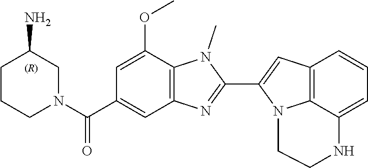 | 5.43 |
| Example 168 | 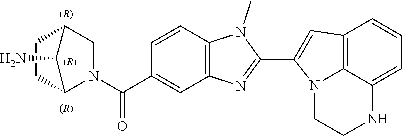 | 2.31 |
| Example 169 | 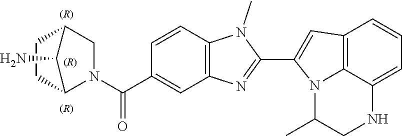 | 0.55 |
| Example 102 | 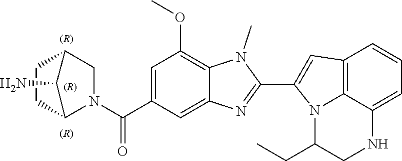 | 0.207 |
| Example 104 | 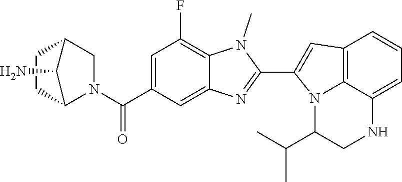 [SMILES: CC(C)C1CNc2cccc3cc(-c4nc5cc(C(=O)N6C[C@H]7CC[C@@H]6[C@@H]7N)cc(F)c5n4C)n1c23] | 0.130 |

### Biological Example 6. Crystallization of PAD4/Example 71 Complex and Structural Determination

1. Purified recombinant PAD4 protein was mixed with Example 71, to a final protein concentration of 4 mg/mL and final compound concentration of 0.5 mM. The mixture was incubated at 4° C. overnight for the formation of PAD4/Example 71 complex.
2. PAD4/Example 71 sample was centrifuged at 13,000 rpm for 10 min to remove precipitation. The supernatant was transferred to a new tube to set up crystal trays.
3. 1 μL PAD4/Example 71 sample was mixed with 1 μL condition containing 9% PEG 3350, 0.1 M HEPES, pH 7.2, 0.1 M Li₂SO₄, on a 24-well hanging drop plate. The plate was put at 18° C. for crystals to grow.
4. The PAD4/Example 71 complex crystals grew to full size in about 4 days. Then they were harvested, snap-cooled in liquid nitrogen (LN₂) and shot at synchrotron.
5. Crystal diffraction data were processed using XDS. Model was built by molecular replacement using phenix, and model refinement was performed in ccp4 suite and phenix.

1. A compound of formula (I0): 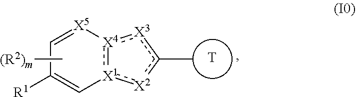
a pharmaceutically acceptable salt, or a stereoisomer thereof, wherein: [symbol]
is a single bond or double bond; provided that 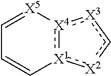
is aromatic;
R¹ is selected from a group consisting of 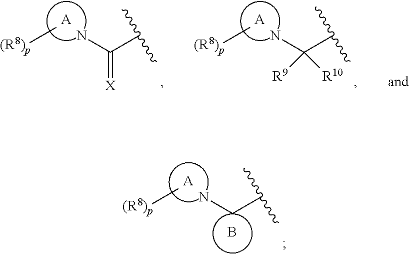
wherein
X is O or S;
ring A is 4-10 membered heterocyclyl or 5-10 membered heteroaryl;
ring B is 3-6 membered monocyclic carbocyclyl; or 3-6 membered monocyclic heterocyclyl;
R² is deuterium, halogen, CN, C₁₋₆alkyl, C₁₋₆alkoxyl, or —NR^{a}R^{b};
X¹ is N or C;
X² is N;
X³ is —N(R³)— or —C(R³)═;
X⁴ is N or C;
X⁵ is N or CH; wherein
R³ is C₁₋₆alkyl, C₁₋₆alkoxyl, C₂₋₆alkenyl, C₂₋₆alkynyl, —NR^{a}R^{b}, —CH₂-3-8 membered cycloalkyl, —CH₂-3-8 membered heterocyclyl, —CH₂-6-10 membered aryl, or —CH₂-5-10 membered heteroaryl; wherein said C₁₋₆alkyl, C₁₋₆alkoxyl, C₂₋₆alkenyl, C₂₋₆alkynyl, cycloalkyl, heterocyclyl, aryl, or heteroaryl represented by R³ or in the group represented by R³ is optionally substituted with one or more groups selected from halogen, oxo, hydroxyl, C₁₋₆ alkyl, haloC₁₋₆ alkyl, hydoxylC₁₋₆ alkyl, methoxylC₁₋₆ alkyl, C₁₋₆ alkoxyl, haloC₁₋₆ alkoxyl, hydoxylC₁₋₆ alkoxyl, methoxylC₁₋₆ alkoxyl, and —NR^{a}R^{b};
ring T is a tricyclic ring selected from the group consisting of 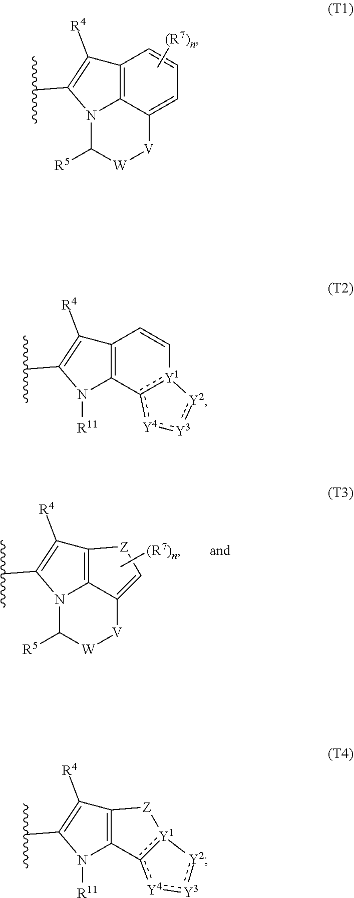
wherein
Z is —O— or —S—;
W is a —(CH₂)ₒ—, —CH(R^{W})—, —C(═O)—, or —CH₂—C(═O)—; wherein o is 1 or 2; R^{W} is C₁₋₆alkyl;
V is —N(R⁶)— or —C(═O)—;
R⁴ is hydrogen, deuterium, halogen, or CN;
R⁵ is hydrogen, C₁₋₆alkyl, haloC₁₋₆alkyl, hydoxylC₁₋₆ alkyl, 3-8 membered cycloalkyl, 3-8 membered heterocyclyl, 6-10 membered aryl, or 5-10 membered heteroaryl; wherein said 3-8 membered cycloalkyl, 3-8 membered heterocyclyl, 6-10 membered aryl, or 5-10 membered heteroaryl represented by R⁵ is optionally substituted with one or more groups selected from halogen, oxo, hydroxyl, C₁₋₆ alkyl, haloC₁₋₆ alkyl, hydoxylC₁₋₆ alkyl, methoxylC₁₋₆ alkyl, C₁₋₆ alkoxyl, haloC₁₋₆ alkoxyl, hydoxylC₁₋₆ alkoxyl, methoxylC₁₋₆ alkoxyl, and —NR^{a}R^{b};
R⁶ is hydrogen, C₁₋₆alkyl, C₁₋₆alkylenehydroxyl, C₁₋₆alkyleneamine, benzoyl, carbonylC₁₋₆alkyl, carbonylC₁₋₆alkylenehydroxyl, C₁₋₆alkyleneamide, C₁₋₆alkylenecarbamate, C₁₋₆alkyleneurea, 3-8 membered cycloalkyl, —CH₂-6-10 membered aryl, or —CH₂-5-10 membered heteroaryl; wherein said C₁₋₆alkyl, C₁₋₆alkylenehydroxyl, C₁₋₆alkyleneamine, benzoyl, carbonylC₁₋₆alkyl, carbonylC₁₋₆alkylenehydroxyl, C₁₋₆alkyleneamide, C₁₋₆alkylenecarbamate, C₁₋₆alkyleneurea, 3-8 membered cycloalkyl, —CH₂-6-10 membered aryl, or —CH₂-5-10 membered heteroaryl represented by R⁶ is optionally substituted with one or more groups selected from halogen, hydroxyl, amino, CN, C₁₋₆alkyl, C₁₋₆alkylcarbonyl, C₁₋₆alkylenehydroxyl, C₁₋₆alkylcarbonylamino, and 3-8 membered cycloalkyl;
R⁷ is deuterium, halogen, cyano, C₁₋₆alkyl, C₁₋₆alkoxy, C₂₋₆alkenyl, C₂₋₆ alkynyl, —NR^{a}R^{b}, —S(═O)₂C₁₋₆alkyl, 3-8 membered cycloalkyl, 3-8 membered heterocyclyl, 6-10 membered aryl, or 5-10 membered heteroaryl; wherein said C₁₋₆alkyl, C₁₋₆alkoxy, C₁₋₆alkenyl, C₁₋₆ alkynyl, 3-8 membered cycloalkyl, 3-8 membered heterocyclyl, 6-10 membered aryl, or 5-10 membered heteroaryl represented by R⁷ is optionally substituted with one or more groups selected from halogen and hydroxyl;
Y¹ is C or N; when Y¹ is C, [symbol] is a double bond; and when Y¹ is N, [symbol] is a single bond;
Y² is —O—, —S—, —S(═O)—, —N(R^{d})—, —C(═O)—, —C(R^{d})₂—, or —C(R^{e})═;
Y³ is —CH₂—, —CH₂—CH₂—, —HC═, —NH—, —N═, —C(═O)—, or —N(R^{f})—CH₂—;
Y⁴ is —NH—, —CH₂—, or —N═; wherein
R^{d} is hydrogen or C₁₋₆alkyl;
R^{e} is hydrogen, halogen, or C₁₋₆alkyl;
R^{f} is hydrogen, C₁₋₆alkyl, —C(═O)C₁₋₆alkyl, or 3-6 membered cycloalkyl;
R¹¹ is —CH₂-3-8 membered cycloalkyl;
R⁸ is halogen, CN, C₁₋₆alkyl, haloC₁₋₆alkyl, C₁₋₆alkoxy, —NR^{a}R^{b}, —NR^{a}C(═O)R^{b}, —NR^{a}C(═O)OR^{b}, —NR^{a}C(═O)NR^{b}, —NR^{a}SO₂R^{b}, —NR^{a}S(═O)(═NR^{b})R^{c}, 3-8 membered carbocyclyl, or 3-8 membered heterocyclyl; or two R₈ groups together with the atoms they attached form 3-8 membered carbocyclyl or 3-8 membered heterocyclyl;
R⁹ and R¹⁰ are independently hydrogen, deuterium, halogen, C₁₋₆alkyl; wherein said C₁₋₆alkyl is optionally substituted with one or more groups selected from halogen, hydroxyl, and methoxyl;
R^{a}, R^{b}, and R^{c} are each independently selected from the group consisting of hydrogen, deuterium, C₁₋₆alkyl, 3-12 membered carbocyclyl, 3-12 membered heterocyclyl, 6-10 membered aryl, and 5-10 membered heteroaryl;
m and n are independently 0, 1, 2, or 3;
p is 0, 1, 2, 3, 4, 5, or 6; and
wherein said heterocyclyl comprises 1-3 heteroatoms selected from oxygen, nitrogen, and sulfur; and said heteroaryl comprises 1-4 heteroatoms selected from oxygen, nitrogen, and sulfur.

2. The compound of claim 1, a pharmaceutically acceptable salt, or a stereoisomer thereof, wherein the compound is represented by formula (I): 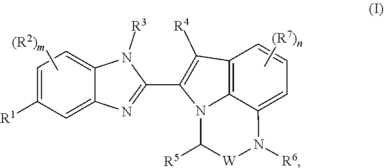
a pharmaceutically acceptable salt, or a stereoisomer thereof, wherein:
W is —(CH₂)ₒ—, —C(═O)—, or —CH₂—C(═O)—; wherein o is 1 or 2;
R⁷ is deuterium, halogen, cyano, C₁₋₆alkyl, C₁₋₆alkoxy, C₂₋₆alkenyl, C₂₋₆ alkynyl, —NR^{a}R^{b}, 3-8 membered cycloalkyl, 3-8 membered heterocyclyl, 6-10 membered aryl, or 5-10 membered heteroaryl; wherein said C₁₋₆alkyl, C₁₋₆alkoxy, C₁₋₆alkenyl, C₁₋₆ alkynyl, 3-8 membered cycloalkyl, 3-8 membered heterocyclyl, 6-10 membered aryl, or 5-10 membered heteroaryl represented by R⁷ is optionally substituted with one or more groups selected from halogen and hydroxyl.

3. The compound of claim 2, a pharmaceutically acceptable salt, or a stereoisomer thereof, wherein
R¹ is 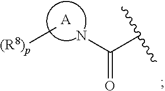
or
R¹ is 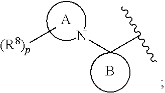
and ring B is 3-4 membered monocyclic heterocyclyl, preferably ring B is oxetanyl; or
R¹ is 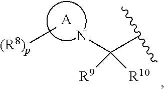
R⁹ and R¹⁰ are independently hydrogen, halo, or haloC₁₋₆alkyl.

4-5. (canceled)

6. The compound of claim 3, a pharmaceutically acceptable salt, or a stereoisomer thereof, wherein W is —CH₂— and/or R⁴ is hydrogen.

7. The compound of claim 6, a pharmaceutically acceptable salt, or a stereoisomer thereof, wherein
ring A is 4-6 membered monocyclic heterocyclyl, 6-9 membered fused heterocyclyl, 6-9 membered bridged heterocyclyl, or 6-9 membered spiro heterocyclyl; and/or
R⁸ is halogen, C₁₋₆alkyl, haloC₁₋₆alkyl, —NR^{a}R^{b}, —NR^{a}(C═O)R^{b}, or —NR^{a}C(═O)OR^{b}; and p is 0, 1, 2, or 3; and/or
R² is halogen, CN, C₁₋₆alkyl, or C₁₋₆alkoxyl; and m is 0, 1, or 2; and/or
R³ is C₁₋₄alkyl, C₁₋₄alkoxyl, C₂₋₄alkynyl, —CH₂-3-5 membered cycloalkyl, —CH₂-3-5 membered heterocyclyl, —CH₂-phenyl, or —CH₂-5-6 membered heteroaryl; wherein said C₁₋₄alkyl, C₁₋₄alkoxyl, C₁₋₄alkynyl, cycloalkyl, heterocyclyl, phenyl, or heteroaryl represented by R³ or in the group represented by R³ is optionally substituted with one to three groups selected from halogen, C₁₋₄ alkyl, hydroxyl, and C₁₋₄alkoxyl; and/or
R⁵ is hydrogen, C₁₋₄alkyl, 3-6 membered cycloalkyl, 3-6 membered heterocyclyl, phenyl, or 5-6 membered heteroaryl, wherein said 3-6 membered cycloalkyl, 3-6 membered heterocyclyl, phenyl, or 5-6 membered heteroaryl represented by R⁵ is optionally substituted with one to three groups selected from halogen, hydroxyl, C₁₋₄ alkyl, haloC₁₋₄ alkyl, hydoxylC₁₋₄ alkyl, methoxylC₁₋₆ alkyl, C₁₋₆ alkoxyl, haloC₁₋₆ alkoxyl, hydoxylC₁₋₆ alkoxyl, methoxylC₁₋₆ alkoxyl, and —NR^{a}R^{b}; and/or
R⁶ is hydrogen, C₁₋₄alkyl, C₁₋₄alkylenehydroxyl, C₁₋₄alkyleneamine, benzoyl, carbonylC₁₋₄alkyl, carbonylC₁₋₄alkylenehydroxyl, C₁₋₄alkyleneamide, C₁₋₄alkylenecarbamate, C₁₋₄alkyleneurea, 3-6 membered cycloalkyl, —CH₂-6 membered aryl, or —CH₂-5-8 membered heteroaryl; wherein said C₁₋₄alkyl, C₁₋₄alkylenehydroxyl, C₁₋₄alkyleneamine, benzoyl, carbonylC₁₋₄alkyl, carbonylC₁₋₄alkylenehydroxyl, C₁₋₄alkyleneamide, C₁₋₄alkylenecarbamate, C₁₋₄alkyleneurea, 3-6 membered cycloalkyl, —CH₂-6 membered aryl, or —CH₂-5-8 membered heteroaryl represented by R⁶ is optionally substituted with one or more groups selected from halogen, hydroxyl, amino, CN, C₁₋₄alkyl, C₁₋₅alkylcarbonyl, C₁₋₄alkylenehydroxyl, C₁₋₄alkylcarbonylamino, and 3-6 membered cycloalkyl; and/or
R⁷ is halogen, cyano, C₁₋₄alkyl, 3-6 membered cycloalkyl, 3-6 membered heterocyclyl, phenyl, or 5-7 membered heteroaryl; wherein said C₁₋₄alkyl, 3-6 membered cycloalkyl, 3-6 membered heterocyclyl, phenyl, or 5-7 membered heteroaryl represented by R⁷ is optionally substituted with one or more halogen; and n is 0 or 1.

8. The compound of claim 6, a pharmaceutically acceptable salt, or a stereoisomer thereof, wherein
ring A is selected from a group consisting of 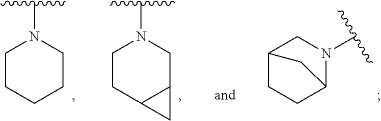 [SMILES: *N1CCCCC1 ; *N1CCC2CC2C1 ; *N1CC2CCC1C2]
R⁸ is halogen, NH₂, or C₁₋₃alkyl; and p is 0, 1, or 2;
R² is —F or —OCH₃; and m is 1;
R³ is C₁₋₂alkyl, C₂₋₃alkynyl, —CH₂-3-4 membered cycloalkyl, —CH₂-3-4 membered heterocyclyl, —CH₂-phenyl, or —CH₂-5 membered heteroaryl; wherein said C₁₋₂alkyl, C₂₋₃alkynyl, cycloalkyl, heterocyclyl, phenyl, or heteroaryl represented by R³ or in the group represented by R³ is optionally substituted with one to three groups selected from halogen, C₁₋₂ alkyl, and C₁₋₂alkoxyl;
R⁵ is hydrogen, C₁₋₃alkyl, or 3-4 membered cycloalkyl;
R⁶ is hydrogen, C₁₋₃alkyl, C₁₋₃alkylenehydroxyl, C₁₋₃alkyleneamine, benzoyl, carbonylC₁₋₃alkyl, carbonylC₁₋₃alkylenehydroxyl, C₁₋₃alkyleneamide, C₁₋₃alkylenecarbamate, C₁₋₃alkyleneurea, 3-5 membered cycloalkyl, —CH₂-6 membered aryl, or —CH₂-5 membered heteroaryl; wherein said hydrogen, C₁₋₃alkyl, C₁₋₃alkylenehydroxyl, C₁₋₃alkyleneamine, benzoyl, carbonylC₁₋₃alkyl, carbonylC₁₋₃alkylenehydroxyl, C₁₋₃alkyleneamide, C₁₋₃alkylenecarbamate, C₁₋₃alkyleneurea, 3-5 membered cycloalkyl, —CH₂-6 membered aryl, or —CH₂-5 membered heteroaryl represented by R⁶ is optionally substituted with one to three groups selected from fluoro, hydroxyl, amino, CN, C₁₋₃alkyl, C₁₋₅alkylcarbonyl, C₁₋₃alkylenehydroxyl, C₁₋₃alkylcarbonylamino, and 3-4 membered cycloalkyl; and
n is 0.

9-14. (canceled)

15. The compound of claim 7, a pharmaceutically acceptable salt, or a stereoisomer thereof, wherein
R³ is selected from a group consisting of 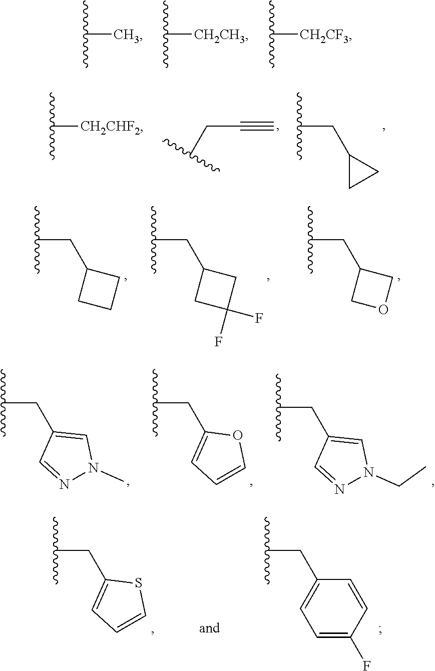
and/or
R⁶ is selected from a group consisting of

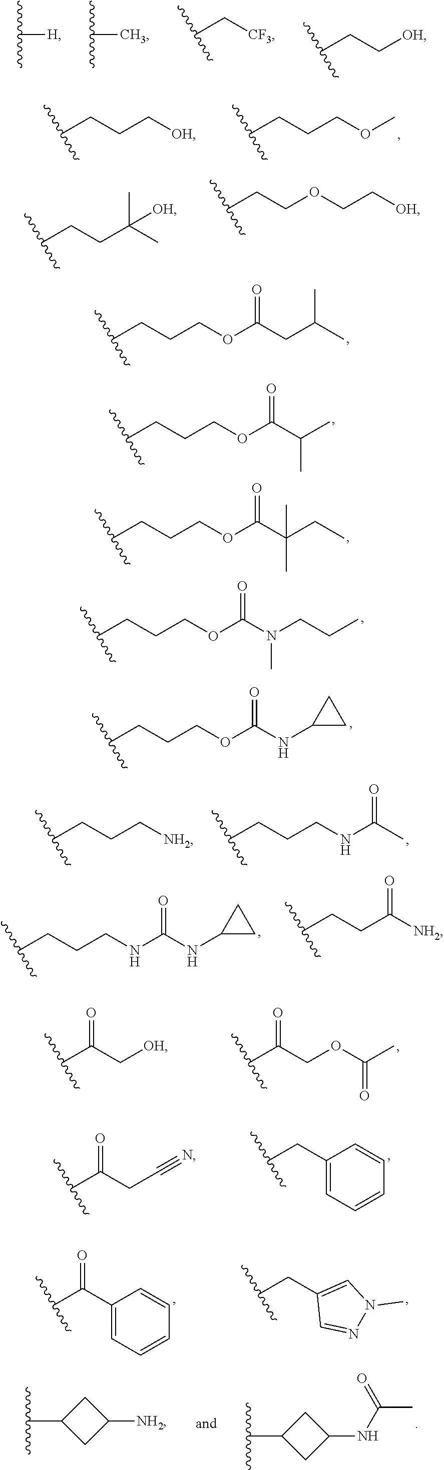

16-24. (canceled)

25. The compound of claim 8, a pharmaceutically acceptable salt, or a stereoisomer thereof, wherein the compound is represented by Formula (II)

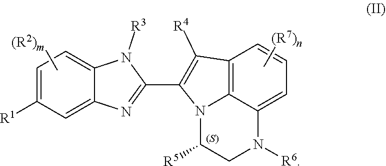

26. The compound of claim 25, a pharmaceutically acceptable salt, or a stereoisomer thereof, wherein R¹ is selected from

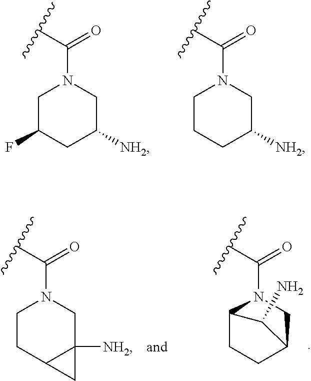

27. The compound of claim 15, a pharmaceutically acceptable salt, or a stereoisomer thereof, wherein R^{a}, R^{b}, and R^{c} are each independently hydrogen or C₁₋₆alkyl.

28. The compound of claim 1, a pharmaceutically acceptable salt, or a stereoisomer thereof, wherein 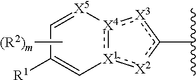
is selected from the group consisting of 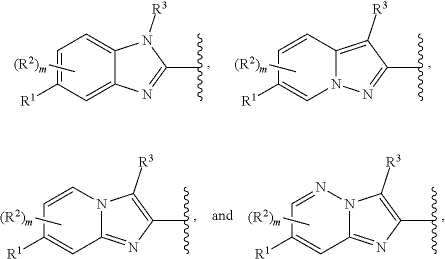
wherein the definition of each variable is defined in claim 1.

29. The compound of claim 1, a pharmaceutically acceptable salt, or a stereoisomer thereof, wherein ring T is represented by Formula (T1) or (T3), 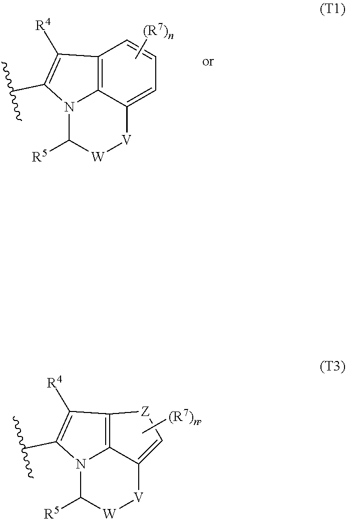
and the definitions of remaining variables are as defined in claim 1.

30-31. (canceled)

32. The compound of claim 1, a pharmaceutically acceptable salt, or a stereoisomer thereof, wherein the compound is represented by Formula (III), 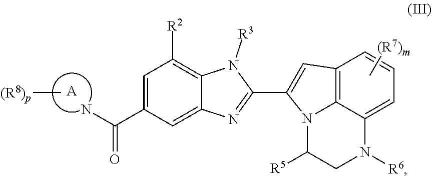
wherein
ring A is selected from the group consisting of 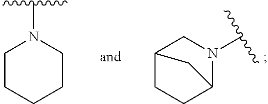 [SMILES: *N1CCCCC1 ; *N1CC2CCC1C2]
R² is halogen, CN, C₁₋₆alkyl, or C₁₋₆alkoxyl;
R³ is C₁₋₆alkyl, C₂₋₆alkynyl, —CH₂-3-5 membered cycloalkyl, —CH₂-3-5 membered heterocyclyl, —CH₂-phenyl, or —CH₂-5 membered heteroaryl; wherein said C₁₋₆alkyl, C₂₋₆alkynyl, cycloalkyl, heterocyclyl, phenyl, or heteroaryl represented by R³ or in the group represented by R³ is optionally substituted with one to three groups selected from halogen and C₁₋₆ alkyl;
R⁵ is hydrogen, C₁₋₃alkyl, or 3-4 membered cycloalkyl;
R⁶ is hydrogen or C₁₋₆alkyl; wherein said C₁₋₆alkyl represented by R⁶ is optionally substituted with one to three groups selected from halogen, hydroxyl, and C₁₋₆alkoxy;
R⁷ is halogen, cyano, C₁₋₆alkyl, haloC₁₋₆alkyl, or —S(═O)₂C₁₋₃alkyl;
R⁸ is halogen or NH₂;
p is 0, 1, or 2; and
n is 0 or 1.

33. The compound of claim 1, a pharmaceutically acceptable salt, or a stereoisomer thereof, wherein the compound is represented by Formula (IIIA), 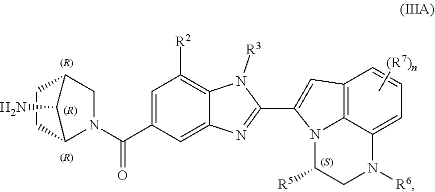
wherein
R² is halogen, CN, C₁₋₆alkyl, or C₁₋₆alkoxyl;
R³ is C₁₋₄alkyl;
R⁵ is hydrogen, C₁₋₃alkyl, or 3-4 membered cycloalkyl;
R⁶ is hydrogen or C₁₋₆alkyl; wherein said C₁₋₆alkyl represented by R⁶ is optionally substituted with one to three groups selected from halogen, hydroxyl, and methoxy;
R⁷ is halogen, cyano, C₁₋₆alkyl, haloC₁₋₆alkyl, or —S(═O)₂C₁₋₃alkyl; and
n is 0 or 1.

34. (canceled)

35. The compound of claim 1, a pharmaceutically acceptable salt, or a stereoisomer thereof, wherein ring T is represented by Formula (T2) or (T4),

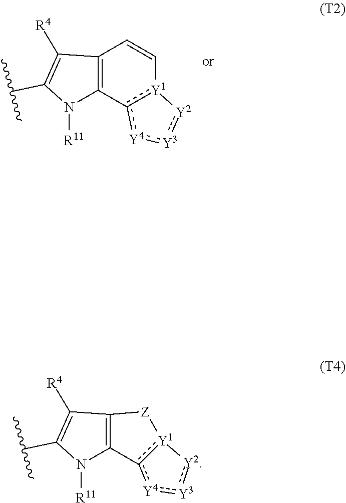

36. The compound of claim 1, a pharmaceutically acceptable salt, or a stereoisomer thereof, wherein the compound is represented by formula (IV), 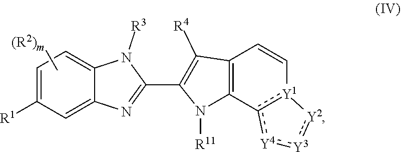
wherein
R¹ is

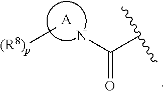

37-53. (canceled)

54. A compound of Table 1, a pharmaceutically acceptable salt, or a stereoisomer thereof.

55. A pharmaceutical composition comprising the compound of claim 1, or a pharmaceutically acceptable salt or a stereoisomer thereof, and a pharmaceutically acceptable carrier or excipient.

56. A method of treating a disease or condition mediated by PAD4 activity, comprising administering to a subject in need thereof, a therapeutically effective amount of the compound of claim 1, or a pharmaceutically acceptable salt, or a stereoisomer thereof.

57. A method for treating a subject with a disease or condition comprising administering to the subject a therapeutically effective amount of the compound of claim 1, or a pharmaceutically acceptable salt or a stereoisomer thereof, wherein said disease or condition is a bacterial infection, a viral infection, a metabolic disease, an autoimmune disease, an auto inflammatory disease, cancer, or a septic condition.

58-59. (canceled)
